(19) United States
(12) Patent Application Publication
(10) Pub. No.: US 2025/0345416 A1
(43) Pub. Date: Nov. 13, 2025

(54) PHARMACEUTICAL COMPOSITIONS FOR DELIVERY OF HERPES SIMPLEX VIRUS ANTIGENS AND RELATED METHODS

(71) Applicants: BioNTech SE, Mainz (DE); The Trustees of the University of Pennsylvania, Philadelphia, PA (US)

(72) Inventors: Ugur Sahin, Mainz (DE); Michael Steven Rooney, Lexington, MA (US); Ekaterina Esaulova, Boston, MA (US); Adam Zuiani, Boston, MA (US); Theresa Addona, Needham, MA (US); Asaf Poran, Stoneham, MA (US); Scott Goulding, Malden, MA (US); Ricardo Sanchez Velazquez, Mainz (DE); Julia Uebele, Darmstadt (DE); Alptekin Güler, Mainz (DE); Gary H. Cohen, Havertown, PA (US); Harvey Friedman, Merion, PA (US); Sita Awasthi, Bala Cynwyd, PA (US)

(21) Appl. No.: 18/833,073

(22) PCT Filed: Jan. 27, 2023
(86) PCT No.: PCT/US23/11789

§ 371 Date: Jul. 25, 2024

Related U.S. Application Data
(60) Provisional application No. 63/303,959, filed on Jan. 27, 2022.
(60) Provisional application No. 63/338,413, filed on May 4, 2022.
(60) Provisional application No. 63/417,679, filed on Oct. 19, 2022.

(51) Int. Cl. A61K 39/245; A61K 9/1272; A61K 9/51; A61K 39/00; C07K 14/005; C07K 14/74; C12N 7/00

(52) U.S. Cl. CPC A61K 39/245; A61K 9/1272; A61K 9/5123; C07K 14/005; C07K 14/70539; C12N 7/00; A61K 2039/51; A61K 2039/55555; A61K 2039/70; C07K 2319/02; C12N 2710/16622; C12N 2710/16634; C12N 2800/22; C12N 2830/50

(57) ABSTRACT

The present disclosure provides pharmaceutical compositions for delivery of HSV antigens (e.g., an HSV vaccine) and related technologies (e.g., components thereof and/or methods relating thereto).

### BACKGROUND

Herpes simplex viruses (HSV), commonly referred to only as herpes, are categorized into two types: herpes simplex virus, type 1 (HSV-1, or oral herpes) and herpes simplex virus, type 2 (HSV-2, or genital herpes). According to the World Health Organization, an estimated 3.7 billion people under age 50 (67% of global population) have HSV-1 infection globally. HSV-1 prevalence is understood as being highest in Africa and lowest in the Americas. An estimated 491 million people aged 15-49 (13% of global population) worldwide have HSV-2 infection. More women are infected with HSV-2 than men, because sexual transmission of HSV is more efficient from men to women than from women to men. Prevalence of HSV-2 infection was estimated to be highest in Africa, followed by the Americas. Prevalence of HSV-2 was also shown to increase with age, though the highest numbers of people newly-infected have historically been in adolescents. Both HSV-1 and HSV-2 infections are lifelong.

### SUMMARY

The present disclosure provides pharmaceutical compositions (e.g., immunogenic compositions, e.g., vaccines) for delivering particular herpes simplex virus (HSV) antigen constructs (e.g., HSV-1 antigen constructs, HSV-2 antigen constructs, or a combination thereof) to a subject (e.g., a patient) and related technologies (e.g., methods). In particular, the present disclosure provides HSV (e.g., HSV-1, HSV-2, or both) vaccine compositions and related technologies (e.g., methods). The present disclosure includes the unexpected discovery that HSV antigens provided in Tables 3-5 below, and antigenic fragments thereof, are particularly advantageous for use in preventing or treating HSV, e.g., in HSV antigen constructs and/or HSV vaccines as further disclosed herein.

The present disclosure provides, for example, polyribonucleotides that encodes one or more HSV antigens (e.g., an HSV-1 antigen, an HSV-2 antigen, or a combination thereof) or antigenic fragments thereof. In some embodiments, such a polyribonucleotide can be part of an RNA construct. In some embodiments, a polyribonucleotide or RNA construct as described herein can be part of a composition (e.g., a pharmaceutical composition, e.g., an immunogenic composition, e.g., a vaccine.

The present disclosure provides a polyribonucleotide encoding a polypeptide. In some embodiments, a polypeptide comprises one or more herpes simplex virus (HSV) antigens or antigenic fragments thereof.

In some embodiments, one or more HSV antigens or antigenic fragments thereof comprise: (i) HSV-1 antigens or antigenic fragments thereof, (ii) HSV-2 antigens or antigenic fragments thereof, or (iii) a combination thereof.

In some embodiments, a polypeptide comprises a single HSV antigen or antigenic fragment thereof. In some embodiments, a polypeptide comprises a single HSV antigen. In some embodiments, a polypeptide comprises a single HSV antigenic fragment.

In some embodiments, the polypeptide comprises two or more HSV antigens or antigenic fragments thereof. In some embodiments, a polypeptide comprises two or more HSV antigens. In some embodiments, a polypeptide comprises two or more HSV antigenic fragments, wherein the two or more HSV antigenic fragments are each a fragment of a different HSV antigen. In some embodiments, a polypeptide comprises two or more HSV antigenic fragments, wherein at least two of the HSV antigenic fragments are a fragment from the same HSV antigen. In some embodiments, a polypeptide comprises three or more HSV antigens or antigenic fragments thereof. In some embodiments, a polypeptide comprises four or more HSV antigens or antigenic fragments thereof.

In some embodiments, a polypeptide does not comprise a full length HSV antigen.

In some embodiments, one or more HSV antigens or antigenic fragments thereof comprise one or more T cell antigens or antigenic fragments thereof. In some embodiments, one or more HSV antigens or antigenic fragments thereof comprise one or more B cell antigens or antigenic fragments thereof.

In some embodiments, one or more HSV antigens or antigenic fragments thereof have at least 80% sequence identity, such as at least 80%, at least 85%, at least 90%, at least 95%, at least 96%, at least 97%, at least 98%, at least 99% or 100% identity with one or more sequences selected from SEQ ID NOs: 1-74 or an antigenic fragment thereof. In some embodiments, a polypeptide comprises one or more HSV-2 antigens or antigenic fragments thereof comprising or consisting of an amino acid sequence that is at least 80%, at least 85%, at least 90%, at least 95%, at least 96%, at least 97%, at least 98%, at least 99% or 100% identical to amino acid sequence selected from SEQ ID NO: 174-196.

In some embodiments, one or more HSV antigens or antigenic fragments thereof comprise: (i) one or more HSV RS1 polypeptides or antigenic fragments thereof, (ii) one or more HSV RL2 polypeptides or antigenic fragments thereof, (iii) one or more HSV UL1 polypeptides or antigenic fragments thereof, (iv) one or more HSV UL5 polypeptides or antigenic fragments thereof, (v) one or more HSV UL9 polypeptides or antigenic fragments thereof, (vi) one or more HSV UL19 polypeptides or antigenic fragments thereof, (vii) one or more HSV UL21 polypeptides or antigenic fragments thereof, (viii) one or more HSV UL25 polypeptides or antigenic fragments thereof, (ix) one or more HSV UL27 polypeptides or antigenic fragments thereof, (x) one or more HSV UL29 polypeptides or antigenic fragments thereof, (xi) one or more HSV UL30 polypeptides or antigenic fragments thereof, (xii) one or more HSV UL39 polypeptides or antigenic fragments thereof, (xiii) one or more HSV UL40 polypeptides or antigenic fragments thereof, (xiv) one or more HSV UL46 polypeptides or antigenic fragments thereof, (xv) one or more HSV UL47 polypeptides or antigenic fragments thereof, (xvi) one or more HSV UL48 polypeptides or antigenic fragments thereof, (xvii) one or more HSV UL49 polypeptides or antigenic fragments thereof, (xviii) one or more HSV UL52 polypeptides or antigenic fragments thereof, (xix) one or more HSV UL54 polypeptides or antigenic fragments thereof, or (xx) a combination thereof. In some embodiments, a polypeptide comprises one or more HSV antigenic fragments, and the one or more HSV antigenic fragments comprise: (i) one or more HSV RS1 polypeptide antigenic fragments, (ii) one or more HSV RL2 polypeptide antigenic fragments, (iii) one or more HSV UL1 polypeptide antigenic fragments, (iv) one or more HSV UL5 polypeptide antigenic fragments, (v) one or more HSV UL9 polypeptide antigenic fragments, (vi) one or more HSV UL19 polypeptide antigenic fragments, (vii) one or more HSV UL21 polypeptide antigenic fragments, (viii) one or more HSV UL25 polypeptide antigenic fragments, (ix) one or more HSV UL27 polypeptide antigenic fragments, (x) one or more HSV UL29 polypeptide antigenic fragments, (xi) one or more HSV UL30 polypeptide antigenic fragments, (xii) one or more HSV UL39 polypeptide antigenic fragments, (xiii) one or more HSV UL40 polypeptide antigenic fragments, (xiv) one or more HSV UL46 polypeptide antigenic fragments, (xv) one or more HSV UL47 polypeptide antigenic fragments, (xvi) one or more HSV UL48 polypeptide antigenic fragments, (xvii) one or more HSV UL49 polypeptide antigenic fragments, (xviii) one or more HSV UL52 polypeptide antigenic fragments, (xix) one or more HSV UL54 polypeptide antigenic fragments, or (xx) a combination thereof.

In some embodiments, a polypeptide comprises one or more HSV RL2 polypeptides or antigenic fragments thereof, one or more HSV RS1 polypeptides or antigenic fragments thereof, and one or more HSV UL54 polypeptides or antigenic fragments thereof. In some embodiments, a polypeptide comprises an HSV-1 gD secretory signal, one or more RL2 polypeptides or antigenic fragments thereof, one or more RS1 polypeptides or antigenic fragments thereof, one or more UL54 polypeptides or antigenic fragments thereof, and a MITD.

In some embodiments, a polypeptide comprises, in N-terminus to C-terminus order, nucleotide sequences that encode an HSV-1 gD secretory signal, an RL2 polypeptide or antigenic fragment thereof, a linker, an RL2 polypeptide or antigenic fragment thereof, a linker, an RS1 polypeptide or antigenic fragment thereof, a linker, a UL54 polypeptide or antigenic fragment thereof, a linker, and a MITD. In some embodiments, a polypeptide comprises or consists of an amino acid sequence that is at least 80%, at least 85%, at least 90%, at least 95%, at least 96%, at least 97%, at least 98%, at least 99%, or 100% identical to amino acid sequence SEQ ID NO: 197.

In some embodiments, a polypeptide comprises, in N-terminus to C-terminus order, nucleotide sequences that encode an HSV-1 gD secretory signal, an UL54 polypeptide or antigenic fragment thereof, a linker, an RS1 polypeptide or antigenic fragment thereof, a linker, an RL2 polypeptide or antigenic fragment thereof, a linker, a RL2 polypeptide or antigenic fragment thereof, a linker, and a MITD. In some embodiments, a polypeptide comprises or consists of an amino acid sequence that is at least 80%, at least 85%, at least 90%, at least 95%, at least 96%, at least 97%, at least 98%, at least 99%, or 100% identical to the amino acid sequence according to SEQ ID NO: 201.

In some embodiments, a polypeptide comprises, in N-terminus to C-terminus order, nucleotide sequences that encode an HSV-2 gD secretory signal, an RL2 polypeptide or antigenic fragment thereof, a linker, an RL2 polypeptide or antigenic fragment thereof, a linker, an RS1 polypeptide or antigenic fragment thereof, a linker, a UL54 polypeptide or antigenic fragment thereof, a linker, and a MITD. In some embodiments, a polypeptide comprises or consists of an amino acid sequence that is at least 80%, at least 85%, at least 90%, at least 95%, at least 96%, at least 97%, at least 98%, at least 99%, or 100% identical to an amino acid sequence according to SEQ ID NO: 205.

In some embodiments, a polypeptide comprises one or more HSV UL29 polypeptides or antigenic fragments thereof, one or more HSV UL39 polypeptides or antigenic fragments thereof, one or more HSV UL49 polypeptides or antigenic fragments thereof, and one or more HSV UL9 polypeptides or antigenic fragments thereof. In some embodiments, a polypeptide comprises an HSV-1 gD secretory signal, one or more HSV UL29 polypeptides or antigenic fragments thereof, one or more HSV UL39 polypeptides or antigenic fragments thereof, one or more HSV UL49 polypeptides or antigenic fragments thereof, one or more HSV UL9 polypeptides or antigenic fragments thereof, and a MITD.

In some embodiments, a polypeptide comprises, in N-terminus to C-terminus order, nucleotide sequences that encode an HSV-1 gD secretory signal, an UL29 polypeptide or antigenic fragment thereof, a linker, an UL39 polypeptide or antigenic fragment thereof, a linker, an UL49 polypeptide or antigenic fragment thereof, a linker, a UL9 polypeptide or antigenic fragment thereof, a linker, and a MITD. In some embodiments, a polypeptide comprises or consists of an amino acid sequence that is at least 80%, at least 85%, at least 90%, at least 95%, at least 96%, at least 97%, at least 98%, at least 99%, or 100% identical to an amino acid sequence according to SEQ ID NO: 198.

In some embodiments, a polypeptide comprises, in N-terminus to C-terminus order, nucleotide sequences that encode an HSV-1 gD secretory signal, an UL9 polypeptide or antigenic fragment thereof, a linker, an UL49 polypeptide or antigenic fragment thereof, a linker, an UL39 polypeptide or antigenic fragment thereof, a linker, a UL29 polypeptide or antigenic fragment thereof, a linker, and a MITD. In some embodiments, a polypeptide comprises or consists of an amino acid sequence that is at least 80%, at least 85%, at least 90%, at least 95%, at least 96%, at least 97%, at least 98%, at least 99%, or 100% identical to an amino acid sequence according to SEQ ID NO: 202.

In some embodiments, a polypeptide comprises one or more HSV UL30 polypeptides or antigenic fragments thereof, one or more HSV UL40 polypeptides or antigenic fragments thereof, one or more HSV UL5 polypeptides or antigenic fragments thereof, and one or more HSV UL52 polypeptides or antigenic fragments thereof. In some embodiments, a polypeptide comprises an HSV-1 gD secretory signal, one or more HSV UL30 polypeptides or antigenic fragments thereof, one or more HSV UL40 polypeptides or antigenic fragments thereof, one or more HSV UL5 polypeptides or antigenic fragments thereof, one or more HSV UL52 polypeptides or antigenic fragments thereof, and a MITD.

In some embodiments, a polypeptide comprises, in N-terminus to C-terminus order, nucleotide sequences that encode an HSV-1 gD secretory signal, an UL30 polypeptide or antigenic fragment thereof, a linker, an UL30 polypeptide or antigenic fragment thereof, a linker, an UL40 polypeptide or antigenic fragment thereof, a linker, a UL5 polypeptide or antigenic fragment thereof, a linker, a UL5 polypeptide or antigenic fragment thereof, a linker, a UL52 polypeptide or antigenic fragment thereof, a linker, and a MITD. In some embodiments, a polypeptide comprises or consists of an amino acid sequence that is at least 80%, at least 85%, at least 90%, at least 95%, at least 96%, at least 97%, at least 98%, at least 99%, or 100% identical to an amino acid sequence according to SEQ ID NO: 199.

In some embodiments, a polypeptide comprises, in N-terminus to C-terminus order, nucleotide sequences that encode an HSV-1 gD secretory signal, an UL52 polypeptide or antigenic fragment thereof, a linker, an UL5 polypeptide or antigenic fragment thereof, a linker, an UL5 polypeptide or antigenic fragment thereof, a linker, a UL40 polypeptide or antigenic fragment thereof, a linker, a UL30 polypeptide or antigenic fragment thereof, a linker, a UL30 polypeptide or antigenic fragment thereof, a linker, and a MITD. In some embodiments, a polypeptide comprises or consists of an amino acid sequence that is at least 80%, at least 85%, at least 90%, at least 95%, at least 96%, at least 97%, at least 98%, at least 99%, or 100% identical to an amino acid sequence according to SEQ ID NO: 203.

In some embodiments, a polypeptide comprises one or more HSV UL1 polypeptides or antigenic fragments thereof, one or more HSV UL19 polypeptides or antigenic fragments thereof, one or more HSV UL21 polypeptides or antigenic fragments thereof, one or more HSV UL27 polypeptides or antigenic fragments thereof, one or more HSV UL46 polypeptides or antigenic fragments thereof, one or more HSV UL47 polypeptides or antigenic fragments thereof, one or more UL48 polypeptides or antigenic fragments thereof, and one or more HSV UL25 polypeptides or antigenic fragments thereof. In some embodiments, a polypeptide comprises an HSV-1 gD secretory signal, one or more HSV UL1 polypeptides or antigenic fragments thereof, one or more HSV UL19 polypeptides or antigenic fragments thereof, one or more HSV UL21 polypeptides or antigenic fragments thereof, one or more HSV UL27 polypeptides or antigenic fragments thereof, one or more HSV UL46 polypeptides or antigenic fragments thereof, one or more HSV UL47 polypeptides or antigenic fragments thereof, one or more UL48 polypeptides or antigenic fragments thereof, one or more HSV UL25 polypeptides or antigenic fragments thereof, and a MITD.

In some embodiments, a polypeptide comprises, in N-terminus to C-terminus order, nucleotide sequences that encode an HSV-1 gD secretory signal, an HSV UL1 polypeptide or antigenic fragment thereof, a linker, an HSV UL19 polypeptide or antigenic fragment thereof, a linker, an HSV UL21 polypeptide or antigenic fragment thereof, a linker, a HSV UL27 polypeptide or antigenic fragment thereof, a linker, a HSV UL27 polypeptide or antigenic fragment thereof, a linker, a HSV UL46 polypeptide or antigenic fragment thereof, a linker, a HSV UL47 polypeptide or antigenic fragment thereof, a linker, a HSV UL25 polypeptide or antigenic fragment thereof, a linker, a HSV UL48 polypeptide or antigenic fragment thereof, a linker, and a MITD. In some embodiments, a polypeptide comprises or consists of an amino acid sequence that is at least 80%, at least 85%, at least 90%, at least 95%, at least 96%, at least 97%, at least 98%, at least 99%, or 100% identical to an amino acid sequence according to SEQ ID NO: 200.

In some embodiments, a polypeptide comprises, in N-terminus to C-terminus order, nucleotide sequences that encode an HSV-1 gD secretory signal, an HSV UL48 polypeptide or antigenic fragment thereof, a linker, an HSV UL25 polypeptide or antigenic fragment thereof, a linker, an HSV UL47 polypeptide or antigenic fragment thereof, a linker, a HSV UL46 polypeptide or antigenic fragment thereof, a linker, a HSV UL27 polypeptide or antigenic fragment thereof, a linker, a HSV UL27 polypeptide or antigenic fragment thereof, a linker, a HSV UL21 polypeptide or antigenic fragment thereof, a linker, a HSV UL19 polypeptide or antigenic fragment thereof, a linker, a HSV UL1 polypeptide or antigenic fragment thereof, a linker, and a MITD. In some embodiments, a polypeptide comprises or consists of an amino acid sequence that is at least 80%, at least 85%, at least 90%, at least 95%, at least 96%, at least 97%, at least 98%, at least 99%, or 100% identical to an amino acid sequence according to SEQ ID NO: 204.

In some embodiments, one or more HSV antigens or antigenic fragments thereof comprise one or more HSV glycoproteins. In some embodiments, one or more HSV glycoproteins comprise an HSV glycoprotein B (gB), an HSV glycoprotein E (gE), an HSV glycoprotein G (gG), an HSV glycoprotein H (gH), an HSV glycoprotein I (gI), an HSV glycoprotein L (gL), or a combination thereof.

In some embodiments, a polypeptide comprises a single HSV antigen. In some embodiments, a single HSV antigen is an HSV glycoprotein. In some embodiments, a HSV glycoprotein is a full-length HSV glycoprotein. In some embodiments, a HSV glycoprotein is an HSV gB, an HSV gE, an HSV gG, an HSV gH, an HSV gI, and an HSV gL.

In some embodiments, a HSV glycoprotein is HSV-2 gB. In some embodiments, a HSV-2 gB is or comprises an amino acid sequence that is at least 80%, at least 85%, at least 90%, at least 95%, at least 96%, at least 97%, at least 98%, at least 99% or 100% identical to SEQ ID NOs: 7, 8, 9, or 74. In some embodiments, a HSV-2 gB consists or comprises an amino acid sequence according to SEQ ID NOs: 7, 8, 9, or 74.

In some embodiments, a HSV glycoprotein is HSV-2 gE. In some embodiments, a HSV-2 gE is or comprises an amino acid sequence that is at least 80%, at least 85%, at least 90%, at least 95%, at least 96%, at least 97%, at least 98%, at least 99% or 100% identical to SEQ ID NOs: 66, 67, 68, or 69. In some embodiments, a HSV-2 gE consists or comprises an amino acid sequence according to SEQ ID NOs: 66, 67, 68, or 69. In some embodiments, a sequence at least 80%, at least 85%, at least 90%, at least 95%, at least 96%, at least 97%, at least 98%, at least 99%, or 100% identical to SEQ ID NOs: 80, 81, 82, 83, or 84.

In some embodiments, a HSV glycoprotein is HSV-2 gH. In some embodiments, a HSV-2 gH is or comprises an amino acid sequence that is at least 80%, at least 85%, at least 90%, at least 95%, at least 96%, at least 97%, at least 98%, at least 99% or 100% identical to SEQ ID NO: 70, 71, 72, or 74. In some embodiments, a HSV-2 gH consists or comprises an amino acid sequence according to SEQ ID NO: 70, 71, 72, or 74.

In some embodiments, a HSV glycoprotein is HSV-2 gI. In some embodiments, a HSV-2 gI is or comprises an amino acid sequence that is at least 80%, 85%, 90%, 91%, 92%, 93%, 94%, 95%, 96%, 97%, 98%, or 99% identical to SEQ ID NO: 62, 63, 64, or 65. In some embodiments, a HSV-2 gI consists or comprises an amino acid sequence according to SEQ ID NO: 62, 63, 64, or 65. In some embodiments, a sequence is at least 80%, at least 85%, at least 90%, at least 95%, at least 96%, at least 97%, at least 98%, at least 99%, or 100% identical to SEQ ID NO: 75, 76, 77, 78, or 79.

In some embodiments, a HSV glycoprotein is HSV-2 gL. In some embodiments, a HSV-2 gL is or comprises an amino acid sequence that is at least 80%, at least 85%, at least 90%, at least 95%, at least 96%, at least 97%, at least 98%, at least 99% or 100% identical to SEQ ID NOs: 58, 59, 60, or 61. In some embodiments, a HSV-2 gL consists or comprises an amino acid sequence according to SEQ ID NOs: 58, 59, 60, or 61.

In some embodiments, a polypeptide comprises a secretory signal. In some embodiments, a secretory signal comprises or consists of a viral secretory signal. In some embodiments, a viral secretory signal comprises or consists of an HSV secretory signal. In some embodiments, a secretory signal is a heterologous secretory signal. In some embodiments, a HSV secretory signal comprises or consists of an HSV-1 or HSV-2 secretory signal.

In some embodiments, a HSV secretory signal is selected from: a) a gD2 secretion signal; b) a gD1 secretion signal; c) a gB1 secretion signal; d) a gI2 secretion signal; e) a gE2 secretion signal; f) a gC2 secretion signal; g) an Eboz secretion signal; h) an IL2 secretion signal; and i) an HLA-DR secretion signal.

In some embodiments, a HSV secretory signal comprises or consists of an HSV gD secretory signal. In some embodiments, a HSV gD secretory signal comprises or consists an amino acid sequence that is at least 80%, at least 85%, at least 90%, at least 95%, at least 96%, at least 97%, at least 98%, at least 99% or 100% identical to SEQ ID NO: 87. In some embodiments, a HSV gD secretory signal comprises or consists of an amino acid sequence that is at least 80%, at least 85%, at least 90%, at least 95%, at least 96%, at least 97%, at least 98%, at least 99% or 100% identical to SEQ ID NO: 88. In some embodiments, a HSV gD secretory signal consists of an amino acid sequence that is at least 80%, at least 85%, at least 90%, at least 95%, at least 96%, at least 97%, at least 98%, at least 99% or 100% identical to SEQ ID NO: 110. In some embodiments, a HSV gD secretory signal consists of an amino acid sequence that is at least 80%, at least 85%, at least 90%, at least 95%, at least 96%, at least 97%, at least 98%, at least 99% or 100% identical to SEQ ID NO: 111.

In some embodiments, a secretory signal is located at the N-terminus of the polypeptide.

In some embodiments, a HSV secretory signal comprises or consists of an HSV-2 glycoprotein I (gI) secretory signal.

In some embodiments, a HSV-2 gI secretory signal comprises an amino acid sequence that is at least 80%, at least 85%, at least 90%, at least 95%, at least 96%, at least 97%, at least 98%, at least 99% or 100% identical to SEQ ID NO: 107.

In some embodiments, a HSV-2 gI secretory signal comprises an amino acid sequence that is at least 80%, at least 85%, at least 90%, at least 95%, at least 96%, at least 97%, at least 98%, at least 99% or 100% identical to SEQ ID NO: 108.

In some embodiments, a polypeptide comprises a transmembrane region. In some embodiments, a transmembrane region comprises or consists of a viral transmembrane region. In some embodiments, a transmembrane region comprises or consists of an HSV transmembrane region. In some embodiments, a HSV transmembrane region comprises or consists of an HSV-1 or HSV-2 transmembrane region. In some embodiments, a HSV transmembrane region comprises or consists of an HSV gD transmembrane region. In some embodiments, a HSV gD transmembrane region consists of an amino acid sequence that is at least 80%, at least 85%, at least 90%, at least 95%, at least 96%, at least 97%, at least 98%, at least 99% or 100% identical to SEQ ID NO: 160.

In some embodiments, a polypeptide does not comprise a transmembrane region.

In some embodiments, a polypeptide comprises a multimerization domain. In some embodiments, a polypeptide comprises one or more linkers. The polyribonucleotide of item 215, wherein the one or more linkers comprise one or more glycine (G) residues and/or one or more serine (S) residues. In some embodiments, one or more linkers comprise or consist of an amino acid sequence according to SEQ ID NO: 163. In some embodiments, one or more linkers comprise or consist of an amino acid sequence that is at least 80%, at least 85%, at least 90%, at least 95%, at least 96%, at least 97%, at least 98%, at least 99% or 100% identical to SEQ ID NO: 165. In some embodiments, one or more linkers comprise or consist of an amino acid sequence that is at least 80%, at least 85%, at least 90%, at least 95%, at least 96%, at least 97%, at least 98%, at least 99% or 100% identical to SEQ ID NO: 168. In some embodiments, one or more linkers comprise or consist of an amino acid sequence that is at least 80%, at least 85%, at least 90%, at least 95%, at least 96%, at least 97%, at least 98%, at least 99% or 100% identical to SEQ ID NO: 217.

In some embodiments, a polyribonucleotide is an isolated polyribonucleotide.

In some embodiments, a polyribonucleotide is an engineered polyribonucleotide.

In some embodiments, a polyribonucleotide is a codon-optimized polyribonucleotide.

The present disclosure also provides an RNA construct.

In some embodiments, an RNA construct comprising in 5′ to 3′ order: (i) a 5′ UTR; (ii) a polyribonucleotide of any according to the present disclosure; (iv) a 3′ UTR; and (v) a polyA tail sequence. In some embodiments, an RNA construct comprises (i) a 5′ UTR that comprises or consists of a modified human alpha-globin 5′-UTR; (ii) a 3′ UTR comprises or consists of a first sequence from the amino terminal enhancer of split (AES) messenger RNA and a second sequence from the mitochondrial encoded 12S ribosomal RNA; or (iii) both.

In some embodiments, a 5′ UTR comprises or consists of a ribonucleic acid sequence that is at least 80%, at least 85%, at least 90%, at least 95%, at least 96%, at least 97%, at least 98%, at least 99% or 100% identical to SEQ ID NO: 208. In some embodiments, a 5′ UTR comprises or consists of a ribonucleic acid sequence that is at least 80%, at least 85%, at least 90%, at least 95%, at least 96%, at least 97%, at least 98%, at least 99% or 100% identical to SEQ ID NO: 209. In some embodiments, a 3′ UTR comprises or consists ribonucleic acid sequence that is at least 80%, at least 85%, at least 90%, at least 95%, at least 96%, at least 97%, at least 98%, at least 99% or 100% identical to SEQ ID NO: 215. In some embodiments, a 3′ UTR comprises or consists of a ribonucleic acid sequence that is at least 80%, at least 85%, at least 90%, at least 95%, at least 96%, at least 97%, at least 98%, at least 99% or 100% identical to SEQ ID NO: 216. In some embodiments, a polyA tail sequence is a split polyA tail sequence. In some embodiments, a split polyA tail sequence consists of a ribonucleic acid sequence that is at least 80%, at least 85%, at least 90%, at least 95%, at least 96%, at least 97%, at least 98%, at least 99% or 100% identical a ribonucleic acid sequence selected from SEQ ID NOs: 210, 212, or 213. In some embodiments, an RNA construct further comprising a 5′ cap. In some embodiments, an RNA construct a cap proximal sequence comprising positions +1, +2, +3, +4, and +5 of the polyribonucleotide. In some embodiments, a 5′ cap comprises or consists of m7(3′OMeG)(5′)ppp(5′)(2′OMeA₁)pG₂, wherein A₁ is position +1 of the polyribonucleotide, and G₂ is position +2 of the polyribonucleotide. In some embodiments, a cap proximal sequence comprises A₁ and G₂ of the Cap1 structure, and a sequence comprising: A₃A₄U₅ (SEQ ID NO: 207) at positions +3, +4 and +5 respectively of the polyribonucleotide.

In some embodiments, a polyribonucleotide includes modified uridines in place of all uridines, optionally wherein modified uridines are each N1-methyl-pseudouridine.

The present disclosure also provides a composition.

In some embodiments, a composition comprises one or more polyribonucleotides according to the present disclosure. In some embodiments, a composition comprises one or more RNA constructs of any one of items 224 to 236. In some embodiments, a composition further comprises lipid nanoparticles, polyplexes (PLX), lipidated polyplexes (LPLX), or liposomes, wherein the one or more polyribonucleotides are fully or partially encapsulated within the lipid nanoparticles, polyplexes (PLX), lipidated polyplexes (LPLX), or liposomes. In some embodiments, a composition further comprises lipid nanoparticles, wherein the one or more polyribonucleotides are encapsulated within the lipid nanoparticles.

The present disclosure also provides a pharmaceutical composition.

In some embodiments, a pharmaceutical composition comprises a composition according to the present disclosure and at least one pharmaceutically acceptable excipient. In some embodiments, a pharmaceutical comprises a cryoprotectant, optionally wherein the cryoprotectant is sucrose. In some embodiments, a pharmaceutical comprises an aqueous buffered solution, optionally wherein the aqueous buffered solution comprises one or more of Tris base, Tris HCl, NaCl, KCl, Na₂HPO₄, and KH₂PO₄.

The present disclosure also provides a combination.

In some embodiments, a combination comprises a first polyribonucleotide according to the present disclosure; and a second polyribonucleotide according to the present disclosure, wherein the first polyribonucleotide and the second polyribonucleotide are different.

In some embodiments, a combination comprises a first pharmaceutical composition comprising a first polyribonucleotide, wherein the first polyribonucleotide is a polyribonucleotide according to the present disclosure; and a second pharmaceutical composition comprising a second polyribonucleotide, wherein the second polyribonucleotide is a polyribonucleotide according to the present disclosure, wherein the first polyribonucleotide and the second polyribonucleotide are different.

In some embodiments, a combination comprises a first polyribonucleotide according to the present disclosure; and a second polyribonucleotide encoding a second polypeptide, wherein the second polypeptide comprises one or more HSV RL2 polypeptides or antigenic fragments thereof, one or more HSV RS1 polypeptides or antigenic fragments thereof, and one or more HSV UL54 polypeptides or antigenic fragments thereof. In some embodiments, a combination comprising: a first pharmaceutical composition comprises a first polyribonucleotide, wherein the first polyribonucleotide is a polyribonucleotide according to the present disclosure; and a second pharmaceutical composition comprising a second polyribonucleotide, wherein the second polyribonucleotide comprises one or more HSV RL2 polypeptides or antigenic fragments thereof, one or more HSV RS1 polypeptides or antigenic fragments thereof, and one or more HSV UL54 polypeptides or antigenic fragments thereof. In some embodiments, a second polypeptide comprises, in N-terminus to C-terminus order, an HSV-1 gD secretory signal, an RL2 polypeptide or antigenic fragment thereof, a linker, an RL2 polypeptide or antigenic fragment thereof, a linker, an RS1 polypeptide or antigenic fragment thereof, a linker, a UL54 polypeptide or antigenic fragment thereof, a linker, and a MITD. In some embodiments, a second polypeptide comprises or consists of an amino acid sequence that is at least 80%, at least 85%, at least 90%, at least 95%, at least 96%, at least 97%, at least 98%, at least 99% or 100% identical to SEQ ID NO: 197. In some embodiments, a second polypeptide comprises, in N-terminus to C-terminus order, an HSV-1 gD secretory signal, an UL54 polypeptide or antigenic fragment thereof, a linker, an RS1 polypeptide or antigenic fragment thereof, a linker, an RL2 polypeptide or antigenic fragment thereof, a linker, a RL2 polypeptide or antigenic fragment thereof, a linker, and a MITD. In some embodiments, a second polypeptide comprises or consists of an amino acid sequence that is at least 80%, at least 85%, at least 90%, at least 95%, at least 96%, at least 97%, at least 98%, at least 99% or 100% identical to SEQ ID NO: 201. In some embodiments, a second polypeptide comprises, in N-terminus to C-terminus order, an HSV-2 gD secretory signal, an RL2 polypeptide or antigenic fragment thereof, a linker, an RL2 polypeptide or antigenic fragment thereof, a linker, an RS1 polypeptide or antigenic fragment thereof, a linker, a UL54 polypeptide or antigenic fragment thereof, a linker, and a MITD. In some embodiments, a second polypeptide comprises or consists of an amino acid sequence that is at least 80%, at least 85%, at least 90%, at least 95%, at least 96%, at least 97%, at least 98%, at least 99% or 100% identical to SEQ ID NO: 205.

In some embodiments, a combination comprises a first polyribonucleotide according to the present disclosure; and a second polyribonucleotide encoding a second polypeptide, wherein the second polypeptide comprises one or more HSV UL29 polypeptides or antigenic fragments thereof, one or more HSV UL39 polypeptides or antigenic fragments thereof, one or more HSV UL49 polypeptides or antigenic fragments thereof, and one or more HSV UL9 polypeptides or antigenic fragments thereof. In some embodiments, a combination comprises a first pharmaceutical composition comprising a first polyribonucleotide, wherein the first polyribonucleotide is a polyribonucleotide according to the present disclosure; and a second pharmaceutical composition comprising a second polyribonucleotide, wherein the second polypeptide comprises one or more HSV UL29 polypeptides or antigenic fragments thereof, one or more HSV UL39 polypeptides or antigenic fragments thereof, one or more HSV UL49 polypeptides or antigenic fragments thereof, and one or more HSV UL9 polypeptides or antigenic fragments thereof. In some embodiments, a second polypeptide comprises, in N-terminus to C-terminus order, an HSV-1 gD secretory signal, an UL29 polypeptide or antigenic fragment thereof, a linker, an UL39 polypeptide or antigenic fragment thereof, a linker, an UL49 polypeptide or antigenic fragment thereof, a linker, a UL9 polypeptide or antigenic fragment thereof, a linker, and a MITD. In some embodiments, a second polypeptide comprises or consists of an amino acid sequence that is at least 80%, at least 85%, at least 90%, at least 95%, at least 96%, at least 97%, at least 98%, at least 99% or 100% identical to SEQ ID NO: 198. In some embodiments, a second polypeptide comprises, in N-terminus to C-terminus order, an HSV-1 gD secretory signal, an UL9 polypeptide or antigenic fragment thereof, a linker, an UL49 polypeptide or antigenic fragment thereof, a linker, an UL39 polypeptide or antigenic fragment thereof, a linker, a UL29 polypeptide or antigenic fragment thereof, a linker, and a MITD. In some embodiments, a second polypeptide comprises or consists of an amino acid sequence that is at least 80%, at least 85%, at least 90%, at least 95%, at least 96%, at least 97%, at least 98%, at least 99% or 100% identical to SEQ ID NO: 202.

In some embodiments, a combination comprises a first polyribonucleotide according to the present disclosure; and a second polyribonucleotide encoding a second polypeptide, wherein the second polypeptide comprises one or more HSV UL30 polypeptides or antigenic fragments thereof, one or more HSV UL40 polypeptides or antigenic fragments thereof, one or more HSV UL5 polypeptides or antigenic fragments thereof, and one or more HSV UL52 polypeptides or antigenic fragments thereof. In some embodiments, a combination comprises a first pharmaceutical composition comprising a first polyribonucleotide, wherein the first polyribonucleotide is a polyribonucleotide according to the present disclosure; and a second pharmaceutical composition comprising a second polyribonucleotide, wherein the second polypeptide comprises one or more HSV UL30 polypeptides or antigenic fragments thereof, one or more HSV UL40 polypeptides or antigenic fragments thereof, one or more HSV UL5 polypeptides or antigenic fragments thereof, and one or more HSV UL52 polypeptides or antigenic fragments thereof. In some embodiments, a second polypeptide comprises, in N-terminus to C-terminus order, an HSV-1 gD secretory signal, an UL30 polypeptide or antigenic fragment thereof, a linker, an UL30 polypeptide or antigenic fragment thereof, a linker, an UL40 polypeptide or antigenic fragment thereof, a linker, a UL5 polypeptide or antigenic fragment thereof, a linker, a UL5 polypeptide or antigenic fragment thereof, a linker, a UL52 polypeptide or antigenic fragment thereof, a linker, and a MITD. In some embodiments, a second polypeptide comprises or consists of an amino acid sequence that is at least 80%, at least 85%, at least 90%, at least 95%, at least 96%, at least 97%, at least 98%, at least 99% or 100% identical to SEQ ID NO: 199. In some embodiments, a second polypeptide comprises, in N-terminus to C-terminus order, an HSV-1 gD secretory signal, an UL52 polypeptide or antigenic fragment thereof, a linker, an UL5 polypeptide or antigenic fragment thereof, a linker, an UL5 polypeptide or antigenic fragment thereof, a linker, a UL40 polypeptide or antigenic fragment thereof, a linker, a UL30 polypeptide or antigenic fragment thereof, a linker, a UL30 polypeptide or antigenic fragment thereof, a linker, and a MITD. In some embodiments, a second polypeptide comprises or consists of an amino acid sequence that is at least 80%, at least 85%, at least 90%, at least 95%, at least 96%, at least 97%, at least 98%, at least 99% or 100% identical to SEQ ID NO: 203.

In some embodiments, a combination comprises a first polyribonucleotide according to the present disclosure; and a second polyribonucleotide encoding a second polypeptide, wherein the second polypeptide comprises one or more HSV UL1 polypeptides or antigenic fragments thereof, one or more HSV UL19 polypeptides or antigenic fragments thereof, one or more HSV UL21 polypeptides or antigenic fragments thereof, one or more HSV UL27 polypeptides or antigenic fragments thereof, one or more HSV UL46 polypeptides or antigenic fragments thereof, one or more HSV UL47 polypeptides or antigenic fragments thereof, one or more UL48 polypeptides or antigenic fragments thereof, and one or more HSV UL25 polypeptides or antigenic fragments thereof. In some embodiments, a combination comprises a first pharmaceutical composition comprising a first polyribonucleotide, wherein the first polyribonucleotide is a polyribonucleotide according to the present disclosure; and a second pharmaceutical composition comprising a second polyribonucleotide, wherein the second polypeptide comprises one or more HSV UL1 polypeptides or antigenic fragments thereof, one or more HSV UL19 polypeptides or antigenic fragments thereof, one or more HSV UL21 polypeptides or antigenic fragments thereof, one or more HSV UL27 polypeptides or antigenic fragments thereof, one or more HSV UL46 polypeptides or antigenic fragments thereof, one or more HSV UL47 polypeptides or antigenic fragments thereof, one or more UL48 polypeptides or antigenic fragments thereof, and one or more HSV UL25 polypeptides or antigenic fragments thereof. In some embodiments, a second polypeptide comprises, in N-terminus to C-terminus order, an HSV-1 gD secretory signal, an HSV UL1 polypeptide or antigenic fragment thereof, a linker, an HSV UL19 polypeptide or antigenic fragment thereof, a linker, an HSV UL21 polypeptide or antigenic fragment thereof, a linker, a HSV UL27 polypeptide or antigenic fragment thereof, a linker, a HSV UL27 polypeptide or antigenic fragment thereof, a linker, a HSV UL46 polypeptide or antigenic fragment thereof, a linker, a HSV UL47 polypeptide or antigenic fragment thereof, a linker, a HSV UL25 polypeptide or antigenic fragment thereof, a linker, a HSV UL48 polypeptide or antigenic fragment thereof, a linker, and a MITD. In some embodiments, a second polypeptide comprises or consists of an amino acid sequence that is at least 80%, at least 85%, at least 90%, at least 95%, at least 96%, at least 97%, at least 98%, at least 99% or 100% identical to SEQ ID NO: 200. In some embodiments, a second polypeptide comprises, in N-terminus to C-terminus order, an HSV-1 gD secretory signal, an HSV UL48 polypeptide or antigenic fragment thereof, a linker, an HSV UL25 polypeptide or antigenic fragment thereof, a linker, an HSV UL47 polypeptide or antigenic fragment thereof, a linker, a HSV UL46 polypeptide or antigenic fragment thereof, a linker, a HSV UL27 polypeptide or antigenic fragment thereof, a linker, a HSV UL27 polypeptide or antigenic fragment thereof, a linker, a HSV UL21 polypeptide or antigenic fragment thereof, a linker, a HSV UL19 polypeptide or antigenic fragment thereof, a linker, a HSV UL1 polypeptide or antigenic fragment thereof, a linker, and a MITD. In some embodiments, a second polypeptide comprises or consists of an amino acid sequence that is at least 80%, at least 85%, at least 90%, at least 95%, at least 96%, at least 97%, at least 98%, at least 99% or 100% identical to SEQ ID NO: 204. In some embodiments, a second polypeptide is an HSV gB. In some embodiments, a second polypeptide consists or comprises an amino acid sequence that is at least 80%, at least 85%, at least 90%, at least 95%, at least 96%, at least 97%, at least 98%, at least 99% or 100% identical to SEQ ID NOs: 7, 8, 9, or 74.

The present disclosure also provides a method that comprises administering a polyribonucleotide according to the present disclosure, or an RNA construct according to the present disclosure, to a subject.

The present disclosure also provides a method that comprises administering a composition according to the present disclosure, to a subject.

The present disclosure also provides a method that comprises administering one or more doses of the composition according to the present disclosure or the pharmaceutical composition according to the present disclosure, to a subject.

The present disclosure also provides a method that comprises administering a combination according to the present disclosure, to a subject.

The present disclosure also provides a pharmaceutical composition according to the present disclosure, for use in the treatment of an HSV infection comprising administering one or more doses of the pharmaceutical composition to a subject.

The present disclosure also provides a pharmaceutical composition according to the present disclosure, for use in the prevention of an HSV infection comprising administering one or more doses of the pharmaceutical composition to a subject.

In some embodiments, a method according to the present disclosure or a pharmaceutical composition according to the present disclosure for use, comprises administering two or more doses of the pharmaceutical composition to a subject.

In some embodiments, a method according to the present disclosure or a pharmaceutical composition for use according to the present disclosure, comprises administering three or more doses of the pharmaceutical composition to a subject.

The present disclosure also provides a method comprising administering a combination according to the present disclosure to a subject. In some embodiments, a first pharmaceutical composition and the second pharmaceutical composition are administered on the same day. In some embodiments, a first pharmaceutical composition and the second pharmaceutical composition are administered on different days. In some embodiments, a first pharmaceutical composition and a second pharmaceutical composition are administered to the subject at different locations on the subject's body. In some embodiments, a method is a method of treating an HSV infection. In some embodiments, a method is a method of preventing an HSV infection. In some embodiments, a subject has or is at risk of developing an HSV infection.

In some embodiments, a subject is a human.

In some embodiments, administration induces an anti-HSV immune response in the subject. In some embodiments, an anti-HSV immune response in the subject comprises an adaptive immune response. In some embodiments, an anti-HSV immune response in the subject comprises a T-cell response. In some embodiments, a T-cell response is or comprises a CD4+ T cell response. In some embodiments, a T-cell response is or comprises a CD8+ T cell response. In some embodiments, an anti-HSV immune system response comprises a B-cell response. In some embodiments, an anti-HSV immune system response comprises the production of antibodies directed against the one or more HSV antigens or antigenic fragments thereof that is at least 80%, at least 85%, at least 90%, at least 95%, at least 96%, at least 97%, at least 98%, at least 99% or 100% identical to one or more sequences selected from SEQ ID NOs: 1-74 or an antigenic fragment thereof.

The present disclosure also provides use of a pharmaceutical composition according to the present disclosure, in the treatment of a herpes simplex virus infection.

The present disclosure also provides use of a pharmaceutical composition according to the present disclosure in the prevention of a herpes simplex virus infection.

The present disclosure also provides use of a pharmaceutical composition according to the present invention, in inducing an anti-herpes simplex immune virus response in a subject.

The present disclosure also provides a polypeptide encoded by a polyribonucleotide according to the present disclosure.

The present disclosure also provides a polypeptide encoded by an RNA construct of any one of items 224 to 236.

The present disclosure also provides a host cell comprising a polyribonucleotide according to the present disclosure.

The present disclosure also provides host cell comprising an RNA construct according to the present disclosure.

The present disclosure also provides host cell comprising a polypeptide according to the present disclosure.

### BRIEF DESCRIPTION OF THE DRAWING

FIG. 1 is a schematic of an HSV particle.

FIG. 2 is a schematic overview of the HSV life cycle. FIG. 2 has been modified from Ibanez, F. J., et al., “Experimental Dissection of the Lytic Replication Cycles of Herpes Simplex Virus in vitro,” Front Microbiol. 2018; 9: 2406, which is incorporated herein by reference in its entirety.

FIG. 3 is a schematic of a model of HSV latent infection. FIG. 3 has been modified from Knipe, D. M., et al., “Clues to mechanisms of herpesviral latent infection and potential cures,” PNAS Sep. 29, 2015 112 (39) 11993-11994, which is incorporated herein by reference in its entirety.

FIG. 4 is a summary table of clinical trial results with HSV vaccine candidates. The table has been modified from Aschner, C. B., & Herold, B. C. (2021), Alphaherpesvirus vaccines. Current Issues in Molecular Biology, 41, 469-508, which is incorporated herein by reference in its entirety.

FIG. 5 is a summary table of HSV-2 vaccine candidates in preclinical development. The table has been modified from Aschner, C. B., & Herold, B. C. (2021), Alphaherpesvirus vaccines. Current Issues in Molecular Biology, 41, 469-508, which is incorporated herein by reference in its entirety.

FIG. 6 is a heat map assessing the phylogeny and homology of HSV-1 and HSV-2 genes. As shown, HSV-1 and HSV-2 genes are homologous with ˜75% sequence identity. HSV-2 demonstrates minimal cross-strain variability.

FIG. 7 includes a line graph depicting the time post HSV infection when intermediate early, early and late gene are expressed.

FIG. 8 is a table showing certain characteristics of data analyzed from Hosken 2006, Jing 2012, and Long 2014, including HSV species, number of subjects, number of genes assayed, experimental methodology, and symptom status of subjects.

FIG. 9 is a graph showing the percent of subjects in data analyzed from Hosken 2006 that were determined to have T cells targeting products of each of 48 analyzed HSV genes, respectively, at a level above the indicated threshold (greater than 20 SFU/10⁶). Data were extracted from the figures of Hosken 2006.

FIG. 10 is a graph showing the percent of subjects in data analyzed from Long 2014 that were determined to have T cells and/or CD4 T cells targeting products of each of 75 analyzed HSV genes, respectively. Data were extracted from the figures of Long 2014.

FIG. 11 is a set of three graphs showing correlation of T cells detected as targeting each of a range of individual HSV genes between pairs of data sets analyzed from literature, in particular between Hosken 2006 and Jing 2012, between Hosken 2006 and Long 2014, or between Jing 2012 and Long 2014. R values are shown for each graph. Hosken 2006/Jing 2012 correlated observed despite different species (Jing 2012 HSV-1, Hosken 2006 HSV-2). No correlation was observed for data from Long 2014 with either of Hosken 2006 or Jing 2012.

FIG. 12 is a chart showing expression levels for each of a range of HSV genes as determined from analysis of a multiple data sets from diverse sources, including human cells, mince, and DRG from latently infected tree shrews. A dashed horizontal line shows determined median expression.

FIG. 13 is a chart plotting data from Hosken 2006 with respect to % of T cells targeting HSV gene products and median expression (see FIG. 12) for each of a variety of HSV genes. Threshold values indicate genes that are immunogenic and well expressed (upper right quadrant based on dashed lines indicating threshold values).

FIG. 14 is a chart plotting data from Jing 2012 with respect to % of T cells targeting HSV gene products and median expression (see FIG. 12) for each of a variety of HSV genes. Threshold values indicate genes that are immunogenic and well expressed (upper right quadrant based on dashed lines indicating threshold values).

FIG. 15 depicts conservation scores determined for amino acids located at positions along an RL2 consensus sequence. For this analysis, complete HSV-1 and HSV-2 genomes were downloaded from VIPR database, and HSV-1 strain 17 and HSV-2 strain HG52 were used as reference strains for HSV-1 and HSV-2 respectively.

FIG. 16 depicts conservation scores determined for amino acids located at positions along an RS1 consensus sequence. For this analysis, complete HSV-1 and HSV-2 genomes were downloaded from VIPR database, and HSV-1 strain 17 and HSV-2 strain HG52 were used as reference strains for HSV-1 and HSV-2 respectively.

FIG. 17 depicts conservation scores determined for amino acids located at positions along an UL19 consensus sequence. For this analysis, complete HSV-1 and HSV-2 genomes were downloaded from VIPR database, and HSV-1 strain 17 and HSV-2 strain HG52 were used as reference strains for HSV-1 and HSV-2 respectively.

FIG. 18 depicts conservation scores determined for amino acids located at positions along an UL1 consensus sequence. For this analysis, complete HSV-1 and HSV-2 genomes were downloaded from VIPR database, and HSV-1 strain 17 and HSV-2 strain HG52 were used as reference strains for HSV-1 and HSV-2 respectively.

FIG. 19 depicts conservation scores determined for amino acids located at positions along an UL21 consensus sequence. For this analysis, complete HSV-1 and HSV-2 genomes were downloaded from VIPR database, and HSV-1 strain 17 and HSV-2 strain HG52 were used as reference strains for HSV-1 and HSV-2 respectively.

FIG. 20 depicts conservation scores determined for amino acids located at positions along an UL25 consensus sequence. For this analysis, complete HSV-1 and HSV-2 genomes were downloaded from VIPR database, and HSV-1 strain 17 and HSV-2 strain HG52 were used as reference strains for HSV-1 and HSV-2 respectively.

FIG. 21 depicts conservation scores determined for amino acids located at positions along an UL27consensus sequence. The UL27 encodes the HSV gB. For this analysis, complete HSV-1 and HSV-2 genomes were downloaded from VIPR database, and HSV-1 strain 17 and HSV-2 strain HG52 were used as reference strains for HSV-1 and HSV-2 respectively.

FIG. 22 depicts conservation scores determined for amino acids located at positions along an UL29 consensus sequence. For this analysis, complete HSV-1 and HSV-2 genomes were downloaded from VIPR database, and HSV-1 strain 17 and HSV-2 strain HG52 were used as reference strains for HSV-1 and HSV-2 respectively.

FIG. 23 depicts conservation scores determined for amino acids located at positions along an UL30 consensus sequence. For this analysis, complete HSV-1 and HSV-2 genomes were downloaded from VIPR database, and HSV-1 strain 17 and HSV-2 strain HG52 were used as reference strains for HSV-1 and HSV-2 respectively.

FIG. 24 depicts conservation scores determined for amino acids located at positions along an UL39 consensus sequence. For this analysis, complete HSV-1 and HSV-2 genomes were downloaded from VIPR database, and HSV-1 strain 17 and HSV-2 strain HG52 were used as reference strains for HSV-1 and HSV-2 respectively.

FIG. 25 depicts conservation scores determined for amino acids located at positions along an UL40 consensus sequence. For this analysis, complete HSV-1 and HSV-2 genomes were downloaded from VIPR database, and HSV-1 strain 17 and HSV-2 strain HG52 were used as reference strains for HSV-1 and HSV-2 respectively.

FIG. 26 depicts conservation scores determined for amino acids located at positions along an UL46 consensus sequence. For this analysis, complete HSV-1 and HSV-2 genomes were downloaded from VIPR database, and HSV-1 strain 17 and HSV-2 strain HG52 were used as reference strains for HSV-1 and HSV-2 respectively.

FIG. 27 depicts conservation scores determined for amino acids located at positions along an UL47 consensus sequence. For this analysis, complete HSV-1 and HSV-2 genomes were downloaded from VIPR database, and HSV-1 strain 17 and HSV-2 strain HG52 were used as reference strains for HSV-1 and HSV-2 respectively.

FIG. 28 depicts conservation scores determined for amino acids located at positions along an UL48 consensus sequence. For this analysis, complete HSV-1 and HSV-2 genomes were downloaded from VIPR database, and HSV-1 strain 17 and HSV-2 strain HG52 were used as reference strains for HSV-1 and HSV-2 respectively.

FIG. 29 depicts conservation scores determined for amino acids located at positions along an UL49 consensus sequence. For this analysis, complete HSV-1 and HSV-2 genomes were downloaded from VIPR database, and HSV-1 strain 17 and HSV-2 strain HG52 were used as reference strains for HSV-1 and HSV-2 respectively.

FIG. 30 depicts conservation scores determined for amino acids located at positions along an UL52 consensus sequence. For this analysis, complete HSV-1 and HSV-2 genomes were downloaded from VIPR database, and HSV-1 strain 17 and HSV-2 strain HG52 were used as reference strains for HSV-1 and HSV-2 respectively.

FIG. 31 depicts conservation scores determined for amino acids located at positions along an UL54 consensus sequence. For this analysis, complete HSV-1 and HSV-2 genomes were downloaded from VIPR database, and HSV-1 strain 17 and HSV-2 strain HG52 were used as reference strains for HSV-1 and HSV-2 respectively.

FIG. 32 depicts conservation scores determined for amino acids located at positions along an UL5 consensus sequence. For this analysis, complete HSV-1 and HSV-2 genomes were downloaded from VIPR database, and HSV-1 strain 17 and HSV-2 strain HG52 were used as reference strains for HSV-1 and HSV-2 respectively.

FIG. 33 depicts conservation scores determined for amino acids located at positions along an UL9 consensus sequence. For this analysis, complete HSV-1 and HSV-2 genomes were downloaded from VIPR database, and HSV-1 strain 17 and HSV-2 strain HG52 were used as reference strains for HSV-1 and HSV-2 respectively.

FIG. 34 depicts HSV strain conservation scores determined for amino acids located at positions along an RL2 consensus sequence.

FIG. 35 depicts HSV strain conservation scores determined for amino acids located at positions along an RS1consensus sequence.

FIG. 36 depicts HSV strain conservation scores determined for amino acids located at positions along an UL19 consensus sequence.

FIG. 37 depicts HSV strain conservation scores determined for amino acids located at positions along an UL1 consensus sequence.

FIG. 38 depicts HSV strain conservation scores determined for amino acids located at positions along an UL21 consensus sequence.

FIG. 39 depicts HSV strain conservation scores determined for amino acids located at positions along an UL25 consensus sequence.

FIG. 40 depicts HSV strain conservation scores determined for amino acids located at positions along an UL27 consensus sequence.

FIG. 41 depicts HSV strain conservation scores determined for amino acids located at positions along an UL29 consensus sequence.

FIG. 42 depicts HSV strain conservation scores determined for amino acids located at positions along an UL30 consensus sequence.

FIG. 43 depicts HSV strain conservation scores determined for amino acids located at positions along an UL39 consensus sequence.

FIG. 44 depicts HSV strain conservation scores determined for amino acids located at positions along an UL40 consensus sequence.

FIG. 45 depicts HSV strain conservation scores determined for amino acids located at positions along an UL46 consensus sequence.

FIG. 46 depicts HSV strain conservation scores determined for amino acids located at positions along an UL47 consensus sequence.

FIG. 47 depicts HSV strain conservation scores determined for amino acids located at positions along an UL48 consensus sequence.

FIG. 48 depicts HSV strain conservation scores determined for amino acids located at positions along an UL49 consensus sequence.

FIG. 49 depicts HSV strain conservation scores determined for amino acids located at positions along an UL52 consensus sequence.

FIG. 50 depicts HSV strain conservation scores determined for amino acids located at positions along an UL54 consensus sequence.

FIG. 51 depicts HSV strain conservation scores determined for amino acids located at positions along an UL5 consensus sequence.

FIG. 52 depicts HSV strain conservation scores determined for amino acids located at positions along an UL9 consensus sequence.

FIG. 53 depicts four HSV antigen constructs A, B, C and D. Construct A includes RL2, RL2, RS1 and UL54 T cell antigens. Construct B includes UL29, UL39, UL49, and UL9 T cell antigens. Construct C includes UL30, UL40, UL5, and UL52 T cell antigens. Construct D includes UL1, UL19, UL21, UL27, UL46, UL47, UL25 and UL48 T cell antigens.

### CERTAIN DEFINITIONS

In general, terminology used herein is in accordance with its understood meaning in the art, unless clearly indicated otherwise. Explicit definitions of certain terms are provided below; meanings of these and other terms in particular instances throughout this specification will be clear to those skilled in the art from context.

In order that the present invention may be more readily understood, certain terms are first defined below. Additional definitions for the following terms and other terms are set forth throughout the specification.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Agent: As used herein, the term “agent”, may refer to a physical entity or phenomenon. In some embodiments, an agent may be characterized by a particular feature and/or effect. In some embodiments, an agent may be a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Amino acid: In its broadest sense, as used herein, the term “amino acid” refers to a compound and/or substance that can be, is, or has been incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Antibody agent: As used herein, the term “antibody agent” refers to an agent that specifically binds to a particular antigen. In some embodiments, the term encompasses a polypeptide or polypeptide complex that includes immunoglobulin structural elements sufficient to confer specific binding. For example, in some embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent in or comprises a polypeptide whose amino acid sequence includes structural elements recognized by those skilled in the art to correspond to CDRs1, 2, and 3 of an antibody variable domain; in some such embodiments, an antibody agent in or comprises a polypeptide or set of polypeptides whose amino acid sequence(s) together include structural elements recognized by those skilled in the art to correspond to both heavy chain and light chain variable region CDRs, e.g., heavy chain CDRs 1, 2, and/or 3 and light chain CDRs 1, 2, and/or 3. In some embodiments, an antibody agent is a polypeptide protein having a binding domain which is homologous or largely homologous to an immunoglobulin-binding domain. In some embodiments, an antibody agent may be or comprise a polyclonal antibody preparation. In some embodiments, an antibody agent may be or comprise a monoclonal antibody preparation. In some embodiments, an antibody agent may include one or more constant region sequences that are characteristic of a particular organism, such as a camel, human, mouse, primate, rabbit, rat; in many embodiments, an antibody agent may include one or more constant region sequences that are characteristic of a human. In some embodiments, an antibody agent may include one or more sequence elements that would be recognized by one skilled in the art as a humanized sequence, a primatized sequence, a chimeric sequence, etc. In some embodiments, an antibody agent may be a canonical antibody (e.g., may comprise two heavy chains and two light chains). In some embodiments, an antibody agent may be in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc.), or other pendant group (e.g., poly-ethylene glycol, etc.)).

Antigen: Those skilled in the art, reading the present specification, will appreciate that the term “antigen” refers to a molecule that is recognized by the immune system, e.g., in particular embodiments the adaptive immune system, such that it elicits an antigen-specific immune response. In some embodiments, an antigen-specific immune response may be or comprise generation of antibodies and/or antigen-specific T cells. In some embodiments, an antigen is a peptide or polypeptide that comprises at least one epitope against which an immune response can be generated. In one embodiment, an antigen is presented by cells of the immune system such as antigen presenting cells like dendritic cells or macrophages. In one embodiments, an antigen or a processed product thereof such as a T-cell antigens is bound by a T- or B-cell receptor, or by an immunoglobulin molecule such as an antibody. Accordingly, an antigen or a processed product thereof may react specifically with antibodies or T lymphocytes (T cells). In one embodiment, an antigen is a parasitic antigen. In accordance with the present disclosure, in some embodiments, an antigen may be delivered by RNA molecules as described herein. In some embodiments, a peptide or polypeptide antigen can be 2-100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids in length. In some embodiments, a peptide or polypeptide antigen can be greater than 50 amino acids. In some embodiments, a peptide or polypeptide antigen can be greater than 100 amino acids. In some embodiments, an antigen is recognized by an immune effector cell. In some embodiments, an antigen if recognized by an immune effector cell is able to induce in the presence of appropriate co-stimulatory signals, stimulation, priming and/or expansion of the immune effector cell carrying an antigen receptor recognizing the antigen. In the context of the embodiments of the present disclosure, in some embodiments, an antigen can be presented or present on the surface of a cell, e.g., an antigen presenting cell. In one embodiment, an antigen is presented by a diseased cell such as a virus-infected cell. In one embodiment, an antigen receptor is a TCR which binds to an epitope of an antigen presented in the context of MHC. In one embodiment, binding of a TCR when expressed by T cells and/or present on T cells to an antigen presented by cells such as antigen presenting cells results in stimulation, priming and/or expansion of said T cells. In one embodiment, binding of a TCR when expressed by T cells and/or present on T cells to an antigen presented on diseased cells results in cytolysis and/or apoptosis of the diseased cells, wherein said T cells preferably release cytotoxic factors, e.g. perforins and granzymes.

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of, susceptibility to, severity of, stage of, etc the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Binding: Those skilled in the art, reading the present specification, will appreciate that the term “binding” typically refers to a non-covalent association between or among entities or moieties. In some embodiments, binding data are expressed in terms of “IC50”. As is understood in the art, IC50 is the concentration of an assessed agent in a binding assay at which 50% inhibition of binding of reference agent known to bind the relevant binding partner is observed. In some embodiments, assays are run under conditions in which the assays are run (e.g., limiting binding target and reference concentrations), these values approximate K_{D} values. Assays for determining binding are well known in the art and are described in detail, for example, in PCT publications WO 94/20127 and WO 94/03205, and other publications such Sidney et al., Current Protocols in Immunology 18.3.1 (1998); Sidney, et al., J. Immunol. 154:247 (1995); and Sette, et al., Mol. Immunol. 31:813 (1994). Alternatively, binding can be expressed relative to binding by a reference standard peptide. For example, can be based on its IC₅₀, relative to the IC₅₀ of a reference standard peptide. Binding can also be determined using other assay systems including those using: live cells (e.g., Ceppellini et al., Nature 339:392 (1989); Christnick et al., Nature 352:67 (1991); Busch et al., Int. Immunol. 2:443 (1990); Hill et al., J. Immunol. 147:189 (1991); del Guercio et al., J. Immunol. 154:685 (1995)), cell free systems using detergent lysates (e.g., Cerundolo et al., J. Immunol 21:2069 (1991)), immobilized purified MHC (e.g., Hill et al., J. Immunol. 152, 2890 (1994); Marshall et al., J. Immunol. 152:4946 (1994)), ELISA systems (e.g., Reay et al., EMBO J. 11:2829 (1992)), surface plasmon resonance (e.g., Khilko et al., J. Biol. Chem. 268:15425 (1993)); high flux soluble phase assays (Hammer et al., J. Exp. Med. 180:2353 (1994)), and measurement of class I MHC stabilization or assembly (e.g., Ljunggren et al., Nature 346:476 (1990); Schumacher et al., Cell 62:563 (1990); Townsend et al., Cell 62:285 (1990); Parker et al., J. Immunol. 149:1896 (1992)).

Cap: As used herein, the term “cap” refers to a structure comprising or essentially consisting of a nucleoside-5′-triphosphate that is typically joined to a 5′-end of an uncapped RNA (e.g., an uncapped RNA having a 5′-diphosphate). In some embodiments, a cap is or comprises a guanine nucleotide. In some embodiments, a cap is or comprises a naturally-occurring RNA 5′ cap, including, e.g., but not limited to a 7-methylguanosine cap, which has a structure designated as “m7G.” In some embodiments, a cap is or comprises a synthetic cap analog that resembles an RNA cap structure and possesses the ability to stabilize RNA if attached thereto, including, e.g., but not limited to anti-reverse cap analogs (ARCAs) known in the art). Those skilled in the art will appreciate that methods for joining a cap to a 5′ end of an RNA are known in the art. For example, in some embodiments, a capped RNA may be obtained by in vitro capping of RNA that has a 5′ triphosphate group or RNA that has a 5′ diphosphate group with a capping enzyme system (including, e.g., but not limited to vaccinia capping enzyme system or Saccharomyces cerevisiae capping enzyme system). Alternatively, a capped RNA can be obtained by in vitro transcription (IVT) of a single-stranded DNA template in the presence of a dinucleotide or trinucleotide cap analog.

Cell-mediated immunity: “Cell-mediated immunity,” “cellular immunity,” “cellular immune response,” or similar terms are meant to include a cellular response directed to cells characterized by expression of an antigen, in particular characterized by presentation of an antigen with class I or class II MHC. A cellular response relates to immune effector cells, in particular to T cells or T lymphocytes which act as either “helpers” or “killers.” The helper T cells (also termed CD4+ T cells or CD4 T cells) play a central role by regulating the immune response and the killer cells (also termed cytotoxic T cells, cytolytic T cells, CD8+ T cells, CD8 T cells, or CTLs) kill diseased cells such as virus-infected cells, preventing the production of more diseased cells.

Co-administration: As used herein, the term “co-administration” refers to use of a pharmaceutical composition (e.g., immunogenic composition, e.g., vaccine) described herein and an additional therapeutic agent. The combined use of a pharmaceutical composition (e.g., immunogenic composition, e.g., vaccine) described herein and an additional therapeutic agent may be performed concurrently or separately (e.g., sequentially in any order). In some embodiments, a pharmaceutical composition (e.g., immunogenic composition, e.g., vaccine) described herein and an additional therapeutic agent may be combined in one pharmaceutically-acceptable carrier, or they may be placed in separate carriers and delivered to a target cell or administered to a subject at different times. Each of these situations is contemplated as falling within the meaning of “co-administration” or “combination,” provided that a pharmaceutical composition (e.g., immunogenic composition, e.g., vaccine) described herein and an additional therapeutic agent are delivered or administered sufficiently close in time that there is at least some temporal overlap in biological effect(s) generated by each on a target cell or a subject being treated.

Codon-optimized: As used herein, the term “codon-optimized” refers to alteration of codons in a coding region of a nucleic acid molecule to reflect the typical codon usage of a host organism without preferably altering the amino acid sequence encoded by the nucleic acid molecule. Within the context of the present disclosure, in some embodiments coding regions are codon-optimized for optimal expression in a subject to be treated using the RNA molecules described herein. In some embodiments, codon-optimization may be performed such that codons for which frequently occurring tRNAs are available are inserted in place of “rare codons.” In some embodiments, codon-optimization may include increasing guanosine/cytosine (G/C) content of a coding region of RNA described herein as compared to the G/C content of the corresponding coding sequence of a wild type RNA, wherein the amino acid sequence encoded by the RNA is preferably not modified compared to the amino acid sequence.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Corresponding to: As used herein, the term “corresponding to” refers to a relationship between two or more entities. For example, the term “corresponding to” may be used to designate the position/identity of a structural element in a compound or composition relative to another compound or composition (e.g., to an appropriate reference compound or composition). For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190 amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure. Those of skill in the art will also appreciate that, in some instances, the term “corresponding to” may be used to describe an event or entity that shares a relevant similarity with another event or entity (e.g., an appropriate reference event or entity). To give but one example, a gene or protein in one organism may be described as “corresponding to” a gene or protein from another organism in order to indicate, in some embodiments, that it plays an analogous role or performs an analogous function and/or that it shows a particular degree of sequence identity or homology, or shares a particular characteristic sequence element.

Derived: In the context of an amino acid sequence (peptide or polypeptide) “derived from” a designated amino acid sequence (peptide or polypeptide), it refers to a structural analogue of a designated amino acid sequence. In some embodiments, an amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

Designed: As used herein, the term “designed” refers to an agent (i) whose structure is or was selected by the hand of man; (ii) that is produced by a process requiring the hand of man; and/or (iii) that is distinct from natural substances and other known agents.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Encode: As used herein, the term “encode” or “encoding” refers to sequence information of a first molecule that guides production of a second molecule having a defined sequence of nucleotides (e.g., mRNA) or a defined sequence of amino acids. For example, a DNA molecule can encode an RNA molecule (e.g., by a transcription process that includes a DNA-dependent RNA polymerase enzyme). An RNA molecule can encode a polypeptide (e.g., by a translation process). Thus, a gene, a cDNA, or an RNA molecule (e.g., an mRNA) encodes a polypeptide if transcription and translation of mRNA corresponding to that gene produces the polypeptide in a cell or other biological system. In some embodiments, a coding region of an RNA molecule encoding a target antigen refers to a coding strand, the nucleotide sequence of which is identical to the mRNA sequence of such a target antigen. In some embodiments, a coding region of an RNA molecule encoding a target antigen refers to a non-coding strand of such a target antigen, which may be used as a template for transcription of a gene or cDNA.

Engineered: In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature.

Epitope: As used herein, the term “epitope” refers to a moiety that is specifically recognized by an immunoglobulin (e.g., antibody or receptor) binding component. For example, an epitope may be recognized by a T cell, a B cell, or an antibody. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized). Accordingly, in some embodiments, an epitope of an antigen may include a continuous or discontinuous fragment of the antigen. In some embodiments, an epitope is or comprises a T cell epitope. In some embodiments, an epitope may have a length of about 5 to about 30 amino acids, or about 10 to about 25 amino acids, or about 5 to about 15 amino acids, or about 5 to 12 amino acids, or about 6 to about 9 amino acids.

Expression: As used herein, the term “expression” of a nucleic acid sequence refers to the generation of a gene product from the nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, etc); (3) translation of an RNA into a polypeptide or protein; and/or (4) post-translational modification of a polypeptide or protein.

Five prime untranslated region: As used herein, the terms “five prime untranslated region” or “5′ UTR” refer to a sequence of an mRNA molecule between a transcription start site and a start codon of a coding region of an RNA. In some embodiments, “5′ UTR” refers to a sequence of an mRNA molecule that begins at a transcription start site and ends one nucleotide (nt) before a start codon (usually AUG) of a coding region of an RNA molecule, e.g., in its natural context.

Fragment: The term “fragment” as used herein in the context of a nucleic acid sequence (e.g. RNA sequence) or an amino acid sequence may typically be a fragment of a reference sequence. In some embodiments, a reference sequence is a full-length sequence of e.g. a nucleic acid sequence or an amino acid sequence. Accordingly, a fragment, typically, refers to a sequence that is identical to a corresponding stretch within a reference sequence. In some embodiments, a fragment comprises a continuous stretch of nucleotides or amino acid residues that corresponds to at least 10%, 20%, 30%, 40%, 50%, 60%, 70%, 80%, 90%, 95% of the total length of a reference sequence from which the fragment is derived. In some embodiments, the term “fragment”, with reference to an amino acid sequence (peptide or polypeptide), relates to a part of an amino acid sequence, e.g., a sequence which represents the amino acid sequence shortened at the N-terminus and/or C-terminus. In some embodiments, a fragment of an amino acid sequence comprises at least 6, in particular at least 8, at least 12, at least 15, at least 20, at least 30, at least 50, or at least 100 consecutive amino acids from an amino acid sequence.

Homology: As used herein, the term “homology” or “homolog” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or polypeptide molecules are considered to be “homologous” to one another if their sequences are at least 15%, 20%, 25%, 30%, 35%, 40%, 45%, 50%, 55%, 60%, 65%, 70%, 75%, 80%, 85%, 90%, 95%, or 99% identical. In some embodiments, polynucleotide molecules (e.g., DNA molecules and/or RNA molecules) and/or polypeptide molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution.

Humoral immunity: As used herein, the term “humoral immunity” or “humoral immune response” refers to antibody production and the accessory processes that accompany it, including: Th2 activation and cytokine production, germinal center formation and isotype switching, affinity maturation and memory cell generation. It also refers to the effector functions of antibodies, which include pathogen neutralization, classical complement activation, and opsonin promotion of phagocytosis and pathogen elimination.

Identity: As used herein, the term “identity” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or between polypeptide molecules are considered to be “substantially identical” to one another if their sequences are at least 25%, 30%, 35%, 40%, 45%, 50%, 55%, 60%, 65%, 70%, 75%, 80%, 85%, 90%, 95%, 96%, 97%, 98%, or 99% identical. Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 75%, at least 80%, at least 85%, at least 90%, at least 95%, at least 96%, at least 97%, at least 98%, at least 99%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller, 1989, which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Immunologically equivalent: The term “immunologically equivalent” means that an immunologically equivalent molecule such as the immunologically equivalent amino acid sequence exhibits the same or essentially the same immunological properties and/or exerts the same or essentially the same immunological effects, e.g., with respect to the type of the immunological effect. In the context of the present disclosure, in some embodiments, the term “immunologically equivalent” is used with respect to the immunological effects or properties of antigens or antigen variants used for immunization. For example, an amino acid sequence is immunologically equivalent to a reference amino acid sequence if said amino acid sequence when exposed to the immune system of a subject induces an immune reaction having a specificity of reacting with the reference amino acid sequence.

In one embodiment, an antigen receptor is an antibody or B cell receptor which binds to an epitope of an antigen. In one embodiment, an antibody or B cell receptor binds to native epitopes of an antigen.

Increased, Induced, or Reduced: As used herein, these terms or grammatically comparable comparative terms, indicate values that are relative to a comparable reference measurement. For example, in some embodiments, an assessed value achieved with a provided pharmaceutical composition (e.g., immunogenic composition, e.g., vaccine) may be “increased” relative to that obtained with a comparable reference pharmaceutical composition (e.g., immunogenic composition, e.g., vaccine). Alternatively or additionally, in some embodiments, an assessed value achieved in a subject may be “increased” relative to that obtained in the same subject under different conditions (e.g., prior to or after an event; or presence or absence of an event such as administration of a pharmaceutical composition (e.g., immunogenic composition, e.g., vaccine) as described herein, or in a different, comparable subject (e.g., in a comparable subject that differs from the subject of interest in prior exposure to a condition, e.g., absence of administration of a pharmaceutical composition (e.g., immunogenic composition, e.g., vaccine) as described herein.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance. In some embodiments, the term “reduced” or equivalent terms refers to a reduction in the level of an assessed value by at least 5%, at least 10%, at least 20%, at least 50%, at least 75% or higher, as compared to a comparable reference. In some embodiments, the term “reduced” or equivalent terms refers to a complete or essentially complete inhibition, i.e., a reduction to zero or essentially to zero. In some embodiments, the term “increased” or “induced” refers to an increase in the level of an assessed value by at least 10%, at least 20%, at least 30%, at least 40%, at least 50%, at least 80%, at least 100%, at least 200%, at least 500%, or higher, as compared to a comparable reference.

Ionizable: The term “ionizable” refers to a compound or group or atom that is charged at a certain pH. In the context of an ionizable amino lipid, such a lipid or a function group or atom thereof bears a positive charge at a certain pH. In some embodiments, an ionizable amino lipid is positively charged at an acidic pH. In some embodiments, an ionizable amino lipid is predominately neutral at physiological pH values, e.g., in some embodiments about 7.0-7.4, but becomes positively charged at lower pH values. In some embodiments, an ionizable amino lipid may have a pKa within a range of about 5 to about 7.

Isolated: The term “isolated” means altered or removed from the natural state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell.

Lipid: As used herein, the terms “lipid” and “lipid-like material” are broadly defined as molecules which comprise one or more hydrophobic moieties or groups and optionally also one or more hydrophilic moieties or groups. Molecules comprising hydrophobic moieties and hydrophilic moieties are also typically denoted as amphiphiles.

RNA lipid nanoparticle: As used herein, the term “RNA lipid nanoparticle” refers to a nanoparticle comprising at least one lipid and RNA molecule(s). In some embodiments, an RNA lipid nanoparticle comprises at least one ionizable amino lipid. In some embodiments, an RNA lipid nanoparticle comprises at least one ionizable amino lipid, at least one helper lipid, and at least one polymer-conjugated lipid (e.g., PEG-conjugated lipid). In various embodiments, RNA lipid nanoparticles as described herein can have an average size (e.g., Z-average) of about 100 nm to 1000 nm, or about 200 nm to 900 nm, or about 200 nm to 800 nm, or about 250 nm to about 700 nm. In some embodiments of the present disclosure, RNA lipid nanoparticles can have a particle size (e.g., Z-average) of about 30 nm to about 200 nm, or about 30 nm to about 150 nm, about 40 nm to about 150 nm, about 50 nm to about 150 nm, about 60 nm to about 130 nm, about 70 nm to about 110 nm, about 70 nm to about 100 nm, about 80 nm to about 100 nm, about 90 nm to about 100 nm, about 70 to about 90 nm, about 80 nm to about 90 nm, or about 70 nm to about 80 nm. In some embodiments, an average size of lipid nanoparticles is determined by measuring the particle diameter. In some embodiments, RNA lipid nanoparticles may be prepared by mixing lipids with RNA molecules described herein.

Lipidoid: As used herein, a “lipidoid” refers to a lipid-like molecule. In some embodiments, a lipoid is an amphiphilic molecule with one or more lipid-like physical properties. In the context of the present disclosure, the term lipid is considered to encompass lipidoids.

Nanoparticle: As used herein, the term “nanoparticle” refers to a particle having an average size suitable for parenteral administration. In some embodiments, a nanoparticle has a longest dimension (e.g., a diameter) of less than 1,000 nanometers (nm). In some embodiments, a nanoparticle may be characterized by a longest dimension (e.g., a diameter) of less than 300 nm. In some embodiments, a nanoparticle may be characterized by a longest dimension (e.g., a diameter) of less than 100 nm. In many embodiments, a nanoparticle may be characterized by a longest dimension between about 1 nm and about 100 nm, or between about 1 μm and about 500 nm, or between about 1 nm and 1,000 nm. In many embodiments, a population of nanoparticles is characterized by an average size (e.g., longest dimension) that is below about 1,000 nm, about 500 nm, about 100 nm, about 50 nm, about 40 nm, about 30 nm, about 20 nm, or about 10 nm and often above about 1 nm. In many embodiments, a nanoparticle may be substantially spherical so that its longest dimension may be its diameter. In some embodiments, a nanoparticle has a diameter of less than 100 nm as defined by the National Institutes of Health.

Naturally occurring: The term “naturally occurring” as used herein refers to an entity that can be found in nature. For example, a peptide or nucleic acid that is present in an organism (including viruses) and can be isolated from a source in nature and which has not been intentionally modified by man in the laboratory is naturally occurring.

Neutralization: As used herein, the term “neutralization” refers to an event in which binding agents such as antibodies bind to a biological active site of a virus such as a receptor binding protein, thereby inhibiting the parasitic infection of cells. In some embodiments, the term “neutralization” refers to an event in which binding agents eliminate or significantly reduce ability of infecting cells.

Nucleic acid particle: A “nucleic acid particle” can be used to deliver nucleic acid to a target site of interest (e.g., cell, tissue, organ, and the like). A nucleic acid particle may comprise at least one cationic or cationically ionizable lipid or lipid-like material, at least one cationic polymer such as protamine, or a mixture thereof and nucleic acid. In some embodiments, a nucleic acid particle is a lipid nanoparticle. In some embodiments, a nucleic acid particle is a lipoplex particle.

Nucleic acid/Polynucleotide: As used herein, the term “nucleic acid” refers to a polymer of at least 10 nucleotides or more. In some embodiments, a nucleic acid is or comprises DNA. In some embodiments, a nucleic acid is or comprises RNA. In some embodiments, a nucleic acid is or comprises peptide nucleic acid (PNA). In some embodiments, a nucleic acid is or comprises a single stranded nucleic acid. In some embodiments, a nucleic acid is or comprises a double-stranded nucleic acid. In some embodiments, a nucleic acid comprises both single and double-stranded fragments. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Nucleotide: As used herein, the term “nucleotide” refers to its art-recognized meaning. When a number of nucleotides is used as an indication of size, e.g., of a polynucleotide, a certain number of nucleotides refers to the number of nucleotides on a single strand, e.g., of a polynucleotide.

Patient: As used herein, the term “patient” refers to any organism who is suffering or at risk of a disease or disorder or condition. Typical patients include animals (e.g., mammals such as mice, rats, rabbits, non-human primates, and/or humans). In some embodiments, a patient is a human. In some embodiments, a patient is suffering from or susceptible to one or more diseases or disorders or conditions. In some embodiments, a patient displays one or more symptoms of a disease or disorder or condition. In some embodiments, a patient has been diagnosed with one or more diseases or disorders or conditions. In some embodiments, a disease or disorder or condition that is amenable to provided technologies is or includes a HSV infection. In some embodiments, a patient is receiving or has received certain therapy to diagnose and/or to treat a disease, disorder, or condition. In some embodiments, a patient is a patient suffering from or susceptible to a HSV infection.

PEG-conjugated lipid: The term “PEG-conjugated lipid” refers to a molecule comprising a lipid portion and a polyethylene glycol portion.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for parenteral administration, for example, by subcutaneous, intramuscular, or intravenous injection as, for example, a sterile solution or suspension formulation.

Pharmaceutically effective amount: The term “pharmaceutically effective amount” or “therapeutically effective amount” refers to the amount which achieves a desired reaction or a desired effect alone or together with further doses. In the case of the treatment of a particular disease, a desired reaction in some embodiments relates to inhibition of the course of the disease. In some embodiments, such inhibition may comprise slowing down the progress of a disease and/or interrupting or reversing the progress of the disease. In some embodiments, a desired reaction in a treatment of a disease may be or comprise delay or prevention of the onset of a disease or a condition. An effective amount of pharmaceutical compositions (e.g., immunogenic compositions, e.g., vaccines) described herein will depend, for example, on a disease or condition to be treated, the severity of such a disease or condition, individual parameters of the patient, including, e.g., age, physiological condition, size and weight, the duration of treatment, the type of an accompanying therapy (if present), the specific route of administration and similar factors. Accordingly, doses of pharmaceutical compositions (e.g., immunogenic compositions, e.g., vaccines) described herein may depend on various of such parameters. In the case that a reaction in a patient is insufficient with an initial dose, higher doses (or effectively higher doses achieved by a different, more localized route of administration) may be used.

Poly(A) sequence: As used herein, the term “poly(A) sequence” or “poly-A tail” refers to an uninterrupted or interrupted sequence of adenylate residues which is typically located at the 3-end of an RNA molecule. Poly(A) sequences are known to those of skill in the art and may follow the 3′-UTR in the RNAs described herein. An uninterrupted poly(A) sequence is characterized by consecutive adenylate residues. In nature, an uninterrupted poly(A) sequence is typical. RNAs disclosed herein can have a poly(A) sequence attached to the free 3-end of the RNA by a template-independent RNA polymerase after transcription or a poly(A) sequence encoded by DNA and transcribed by a template-dependent RNA polymerase.

Polypeptide: As used herein, the term “polypeptide” refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications comprise acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide.

Prevent: As used herein, the term “prevent” or “prevention” when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Recombinant: The term “recombinant” in the context of the present disclosure means “made through genetic engineering”. In some embodiments, a “recombinant” entity such as a recombinant nucleic acid in the context of the present disclosure is not naturally occurring.

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Ribonucleic acid (RNA): As used herein, the term “RNA” refers to a polymer of ribonucleotides. In some embodiments, an RNA is single stranded. In some embodiments, an RNA is double stranded. In some embodiments, an RNA comprises both single and double stranded fragments. In some embodiments, an RNA can comprise a backbone structure as described in the definition of “Nucleic acid/Polynucleotide” above. An RNA can be a regulatory RNA (e.g., siRNA, microRNA, etc.), or a messenger RNA (mRNA). In some embodiments where an RNA is a mRNA. In some embodiments where an RNA is a mRNA, a RNA typically comprises at its 3′ end a poly(A) region. In some embodiments where an RNA is a mRNA, an RNA typically comprises at its 5′ end an art-recognized cap structure, e.g., for recognizing and attachment of a mRNA to a ribosome to initiate translation. In some embodiments, a RNA is a synthetic RNA. Synthetic RNAs include RNAs that are synthesized in vitro (e.g., by enzymatic synthesis methods and/or by chemical synthesis methods).

Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. The term “ribonucleotide” also encompasses ribonucleotide triphosphates including modified and non-modified ribonucleotide triphosphates.

Risk: As will be understood from context, “risk” of a disease, disorder, and/or condition refers to a likelihood that a particular individual will develop the disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from individuals comparable to a particular individual. In some embodiments, relative risk is 0, 1, 2, 3, 4, 5, 6, 7, 8, 9, 10, or more. In some embodiments, risk may reflect one or more genetic attributes, e.g., which may predispose an individual toward development (or not) of a particular disease, disorder and/or condition. In some embodiments, risk may reflect one or more epigenetic events or attributes and/or one or more lifestyle or environmental events or attributes.

RNA lipoplex particle: As used herein, the term “RNA lipoplex particle” refers to a complex comprising liposomes, in particular cationic liposomes, and RNA molecules. Without wishing to bound by a particular theory, electrostatic interactions between positively charged liposomes and negatively charged RNA results in complexation and spontaneous formation of RNA lipoplex particles. In some embodiments, positively charged liposomes may comprise a cationic lipid, such as in some embodiments DOTMA, and additional lipids, such as in some embodiments DOPE. In one embodiment, a RNA lipoplex particle is a nanoparticle.

Selective or specific: The term “selective” or “specific”, when used herein in reference to an agent having an activity, is understood by those skilled in the art to mean that the agent discriminates between potential target entities, states, or cells. For example, in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of a target-binding moiety for one or more other potential target entities (e.g., competitors). In some embodiments, specificity is evaluated relative to that of a reference specific binding moiety. In some embodiments, specificity is evaluated relative to that of a reference non-specific binding moiety.

Stable: As used herein, the term “stable” in the context of the present disclosure refers to a pharmaceutical composition (e.g., immunogenic composition, e.g., vaccine) as a whole and/or components thereof meeting or exceeding pre-determined acceptance criteria. For example, in some embodiments, a stable pharmaceutical composition (e.g., immunogenic composition, e.g., vaccine) exhibits no unacceptable levels of microbial growth, and substantially no or no breakdown or degradation of the active biological molecule component(s). In some embodiments, a stable pharmaceutical composition (e.g., immunogenic composition, e.g., vaccine) refers to the integrity of RNA molecules being maintained at least above 90% or more. In some embodiments, a stable pharmaceutical composition (e.g., immunogenic composition, e.g., vaccine) refers to at least 90% or more (including, e.g., at least 95%, at least 96%, at least 97%, or more) of RNA molecules being maintained to be encapsulated within lipid nanoparticles. In some embodiments, a stable pharmaceutical composition (e.g., immunogenic composition, e.g., vaccine) refers to a formulation that remains capable of eliciting a desired immunologic response when administered to a subject. In some embodiments, a pharmaceutical composition (e.g., immunogenic composition, e.g., vaccine) remains stable for a specified period of time under certain conditions.

Subject: As used herein, the term “subject” refers to an organism to be administered with a composition described herein, e.g., for experimental, diagnostic, prophylactic, and/or therapeutic purposes. Typical subjects include animals (e.g., mammals such as mice, rats, rabbits, non-human primates, domestic pets, etc.) and humans. In some embodiments, a subject is a human subject. In some embodiments, a subject is suffering from a disease, disorder, or condition (e.g., a HSV infection). In some embodiments, a subject is susceptible to a disease, disorder, or condition (e.g., a HSV infection). In some embodiments, a subject displays one or more symptoms or characteristics of a disease, disorder, or condition (e.g., a HSV infection). In some embodiments, a subject displays one or more non-specific symptoms of a disease, disorder, or condition (e.g., a HSV infection). In some embodiments, a subject does not display any symptom or characteristic of a disease, disorder, or condition (e.g., a HSV infection). In some embodiments, a subject is someone with one or more features characteristic of susceptibility to or risk of a disease, disorder, or condition (e.g., a HSV infection). In some embodiments, a subject is a patient. In some embodiments, a subject is an individual to whom diagnosis and/or therapy is and/or has been administered.

Suffering from: An individual who is “suffering from” a disease, disorder, and/or condition has been diagnosed with and/or displays one or more symptoms of a disease, disorder, and/or condition.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synthetic: As used herein, the term “synthetic” refers to an entity that is artificial, or that is made with human intervention, or that results from synthesis rather than naturally occurring. For example, in some embodiments, a synthetic nucleic acid or polynucleotide refers to a nucleic acid molecule that is chemically synthesized, e.g., in some embodiments by solid-phase synthesis. In some embodiments, the term “synthetic” refers to an entity that is made outside of biological cells. For example, in some embodiments, a synthetic nucleic acid or polynucleotide refers to a nucleic acid molecule (e.g., an RNA) that is produced by in vitro transcription using a template.

Therapy: The term “therapy” refers to an administration or delivery of an agent or intervention that has a therapeutic effect and/or elicits a desired biological and/or pharmacological effect (e.g., has been demonstrated to be statistically likely to have such effect when administered to a relevant population). In some embodiments, a therapeutic agent or therapy is any substance that can be used to alleviate, ameliorate, relieve, inhibit, prevent, delay onset of, reduce severity of, and/or reduce incidence of one or more symptoms or features of a disease, disorder, and/or condition. In some embodiments, a therapeutic agent or therapy is a medical intervention (e.g., surgery, radiation, phototherapy) that can be performed to alleviate, relieve, inhibit, present, delay onset of, reduce severity of, and/or reduce incidence of one or more symptoms or features of a disease, disorder, and/or condition.

Three prime untranslated region: As used herein, the terms “three prime untranslated region” or “3′ UTR” refer to a sequence of an mRNA molecule that begins following a stop codon of a coding region of an open reading frame sequence. In some embodiments, the 3′ UTR begins immediately after a stop codon of a coding region of an open reading frame sequence, e.g., in its natural context. In other embodiments, the 3′ UTR does not begin immediately after stop codon of the coding region of an open reading frame sequence, e.g., in its natural context.

Threshold level (e.g., acceptance criteria): As used herein, the term “threshold level” refers to a level that are used as a reference to attain information on and/or classify the results of a measurement, for example, the results of a measurement attained in an assay. For example, in some embodiments, a threshold level means a value measured in an assay that defines the dividing line between two subsets of a population (e.g. a batch that satisfy quality control criteria vs. a batch that does not satisfy quality control criteria). Thus, a value that is equal to or higher than the threshold level defines one subset of the population, and a value that is lower than the threshold level defines the other subset of the population. A threshold level can be determined based on one or more control samples or across a population of control samples. A threshold level can be determined prior to, concurrently with, or after the measurement of interest is taken. In some embodiments, a threshold level can be a range of values.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition. In some embodiments, treatment may be administered to a subject at a later-stage of disease, disorder, and/or condition.

Vaccination: As used herein, the term “vaccination” refers to the administration of a composition intended to generate an immune response, for example to a disease-associated (e.g., disease-causing) agent. In some embodiments, vaccination can be administered before, during, and/or after exposure to a disease-associated agent, and in certain embodiments, before, during, and/or shortly after exposure to the agent. In some embodiments, vaccination includes multiple administrations, appropriately spaced in time, of a vaccine composition. In some embodiments, vaccination generates an immune response to an infectious agent.

Vaccine: As used herein, the term “vaccine” refers to a composition that induces an immune response upon administration to a subject. In some embodiments, an induced immune response provides protective immunity.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature.

Vector: as used herein, refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In some embodiments, known techniques may be used, for example, for generation or manipulation of recombinant DNA, for oligonucleotide synthesis, and for tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (4th ed., Cold Spring Harbor Laboratory Press, Cold Spring Harbor, N.Y. (2012)), which is incorporated herein by reference for any purpose.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls. The section headings used herein are for organizational purposes only and are not to be construed as limiting the subject matter described in any way.

### DETAILED DESCRIPTION OF CERTAIN EMBODIMENTS

As discussed above, the present disclosure provides pharmaceutical compositions (e.g., immunogenic compositions, e.g., vaccines) for delivering particular herpes simplex virus (HSV) antigen constructs (e.g., HSV-1 antigen constructs, HSV-2 antigen constructs, or a combination thereof) to a subject (e.g., a patient) and related technologies (e.g., methods). In particular, the present disclosure provides HSV (e.g., HSV-1, HSV-2, or both) vaccine compositions and related technologies (e.g., methods).

The present disclosure provides for example, polyribonucleotides that encode one or more HSV antigens. In some embodiments, such a polyribonucleotide can be part of an RNA construct. In some embodiments, a polyribonucleotide or RNA construct as described herein can be part of a composition (e.g., a pharmaceutical composition, e.g., an immunogenic composition, e.g., a vaccine.

In some embodiments, technologies provided herein are directed against HSV. A description of HSV and certain exemplary features is described below.

### I. Herpes Simplex Virus (HSV)

Herpes simplex virus (HSV) belongs to the alpha subfamily of the human herpesvirus family and includes HSV-1 and HSV-2. The structure of HSV-1 and HSV-2 mainly include (from inside to outside) a DNA core, capsid, tegument and envelope. Each of HSV-1 and HSV-2 have a double stranded DNA genome of about 153 kb, encoding at least 80 genes. The DNA core is enclosed by an icosapentahedral capsid composed of 162 capsomeres, 150 hexons and 12 pentons, made of six different viral proteins. The DNA is surrounded by at least 20 different viral tegument proteins that have structural and regulatory roles. Some of them participating in capsid transport to the nucleus and other organelles, viral DNA entry into the nucleus, activation of early genes transcription, suppression of cellular protein biosynthesis, and mRNA degradation. The viral envelope surrounding the tegument has at least 12 different glycoproteins (B-N) on their surface. The glycoproteins may exist as heterodimers (H/L and E/I) with most existing as monomers.

HSV-1 and HSV-2 are responsible for a number of minor, moderate and severe pathologies, including oral and genital ulceration, virally induced blindness, viral encephalitis and disseminated infection of neonates. HSV-1 and HSV-2 are usually transmitted by different routes and affect different areas of the body, but the signs and symptoms that they cause can overlap. Infections caused by HSV-1 represent one of the more widespread infections of the orofacial region and commonly causes herpes labialis, herpetic stomatitis, and keratitis. HSV-2 typically causes genital herpes and is transmitted primarily by direct sexual contact with lesions. Most genital HSV infections are caused by HSV-2, however, an increasing number of genital HSV infections have been attributed to HSV-1. Genital HSV-1 infections are typically less severe and less prone to occurrence than genital HSV-2 infections.

HSV infections are transmitted through contact with herpetic lesions, mucosal surfaces, genital secretions, or oral secretions. The average incubation period after exposure is typically 4 days, but may range between 2 and 12 days. HSV particles can infect neuronal prolongations enervating peripheral tissues and establish latency in these cells, namely in the trigeminal ganglia and dorsal root ganglia of the sacral area from where they can sporadically reactivate. Additionally, similar to other herpesviruses, HSV infections are lifelong and generally asymptomatic. Without wishing to be bound by any particular theory, it is understood that HSV particles can be shed from infected individuals independent of the occurrence of clinical manifestations.

HSV infections are rarely fatal, but are characterized by blisters that can rupture and become painful. There are few clear differences in clinical presentation based on the type of infecting virus. However, as discussed above, HSV-1 infections tend to be less severe than HSV-2 infections, and patients infected with HSV-2 generally have more outbreaks.

### A. Lifecycle

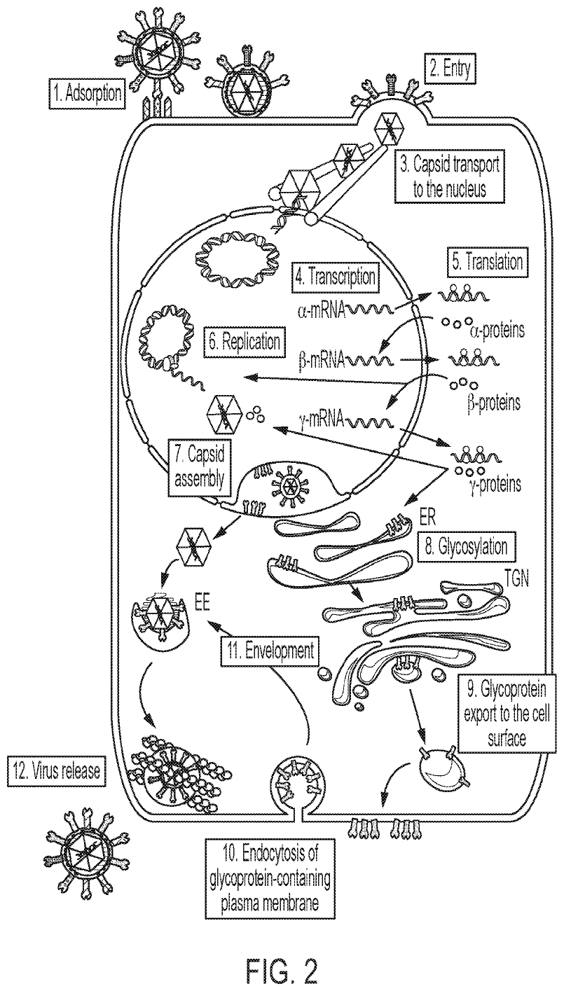

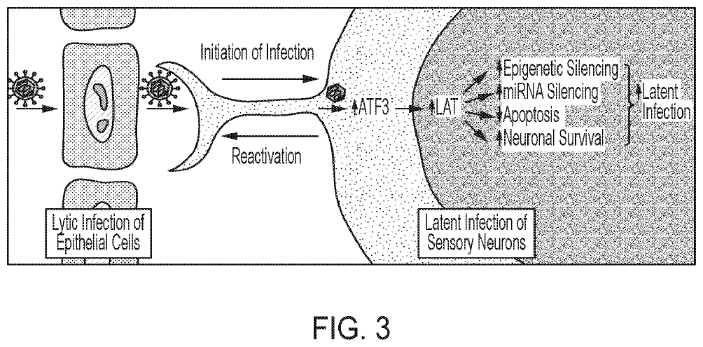

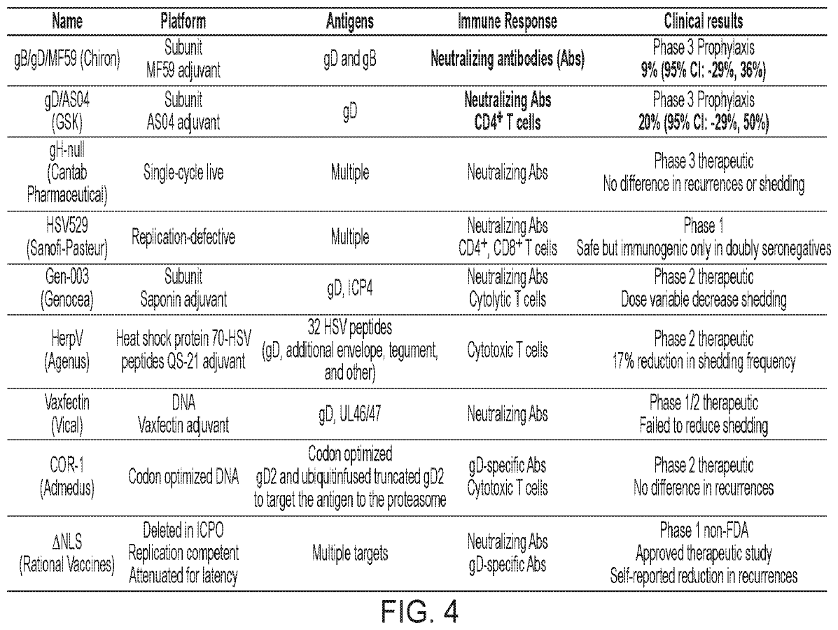

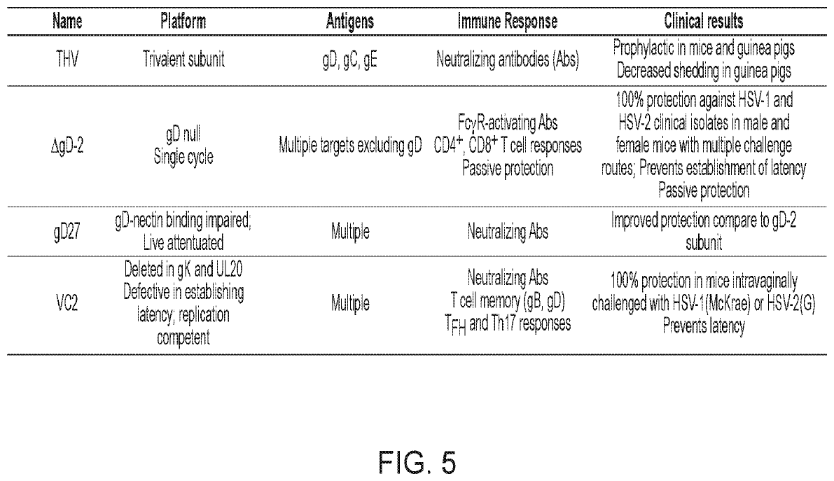

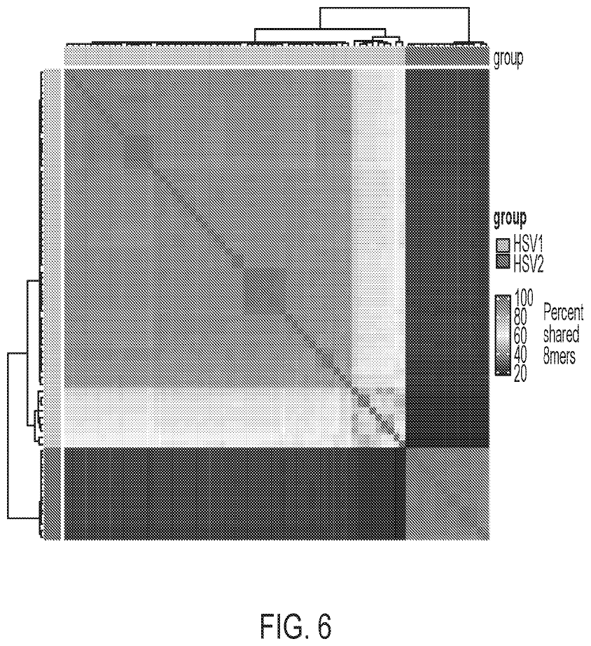

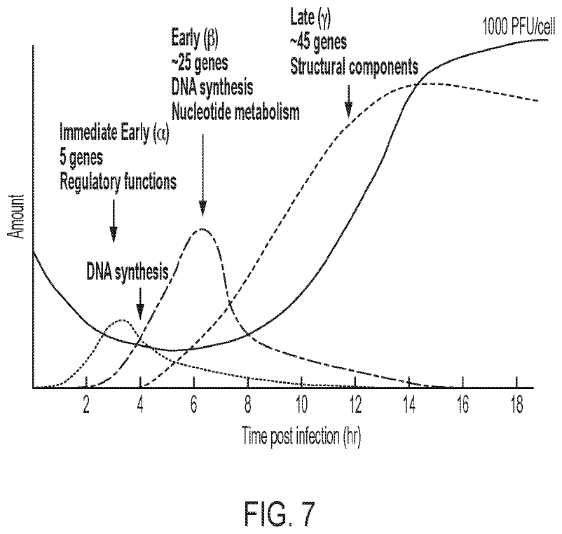

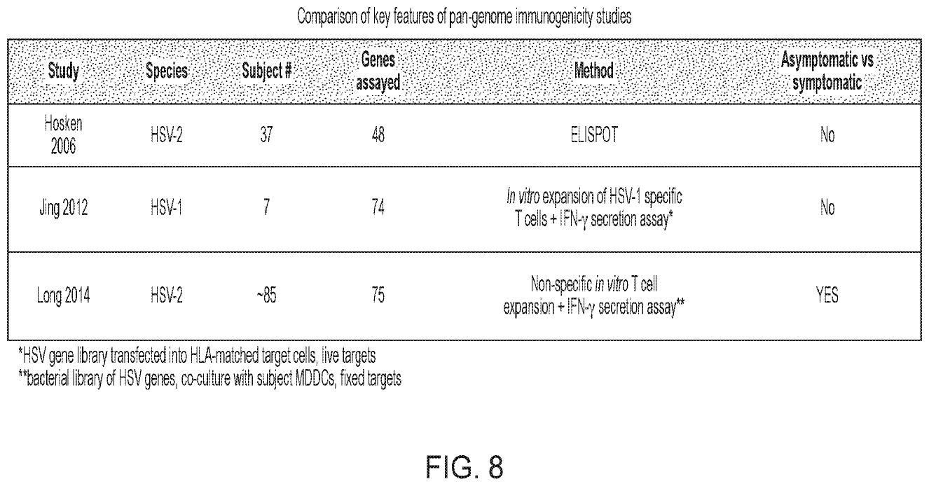

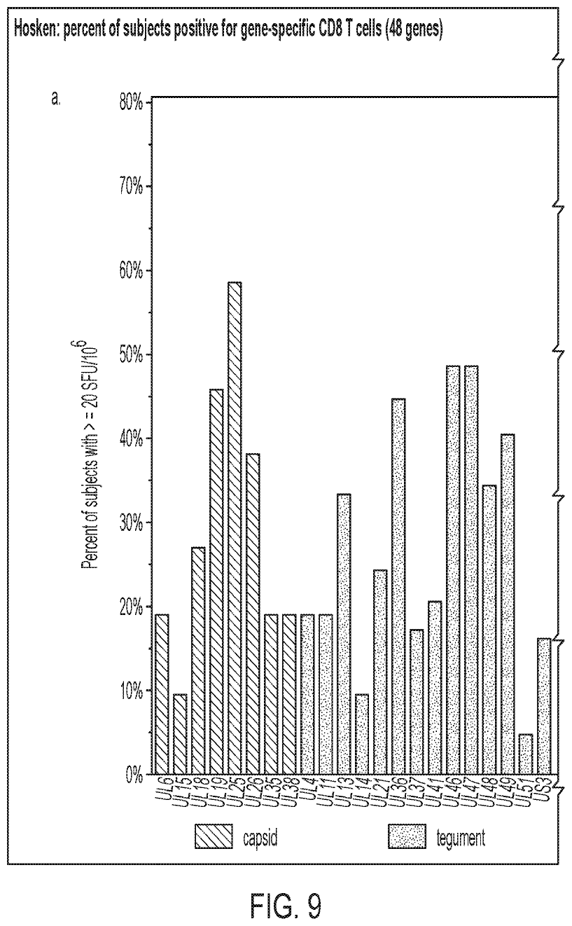

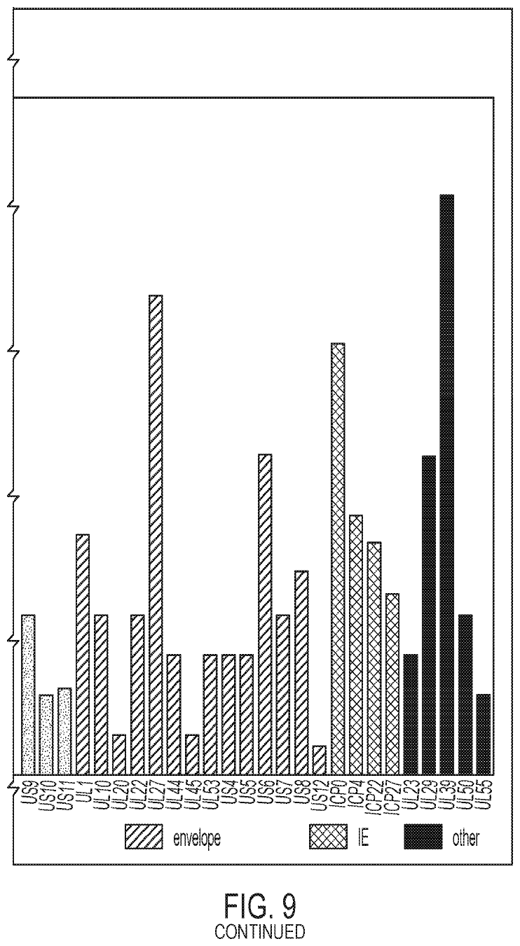

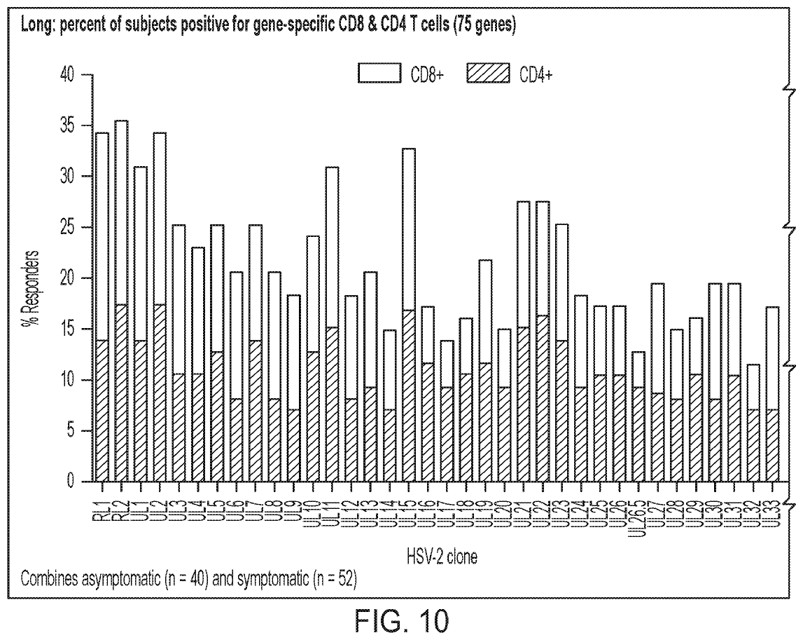

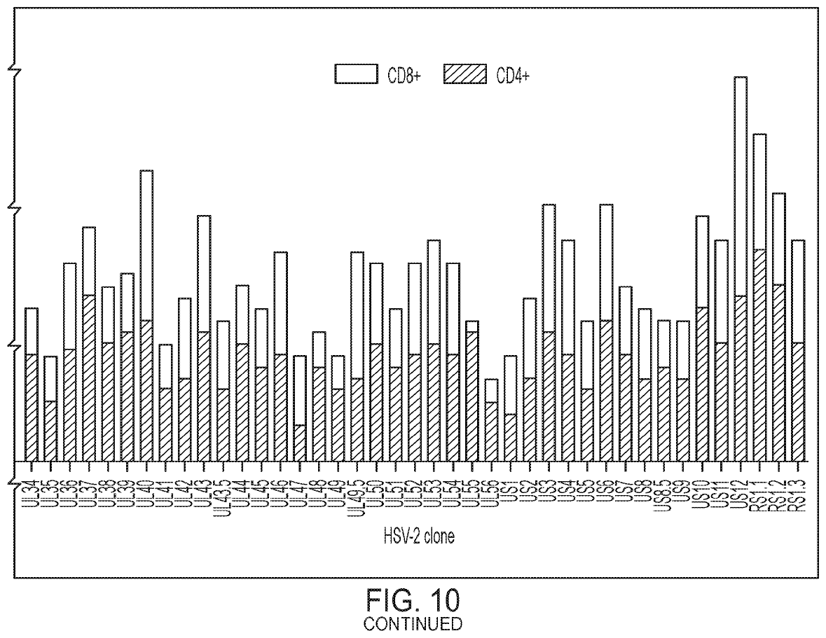

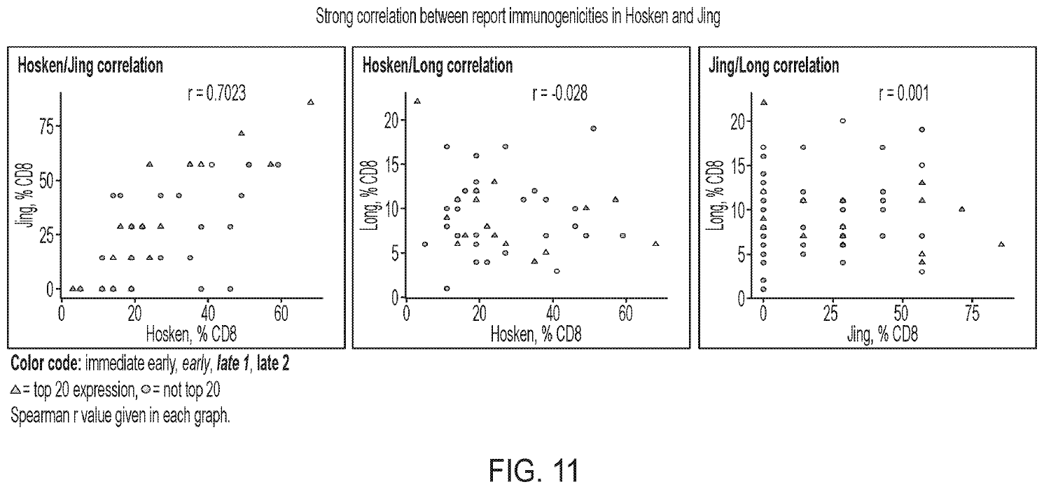

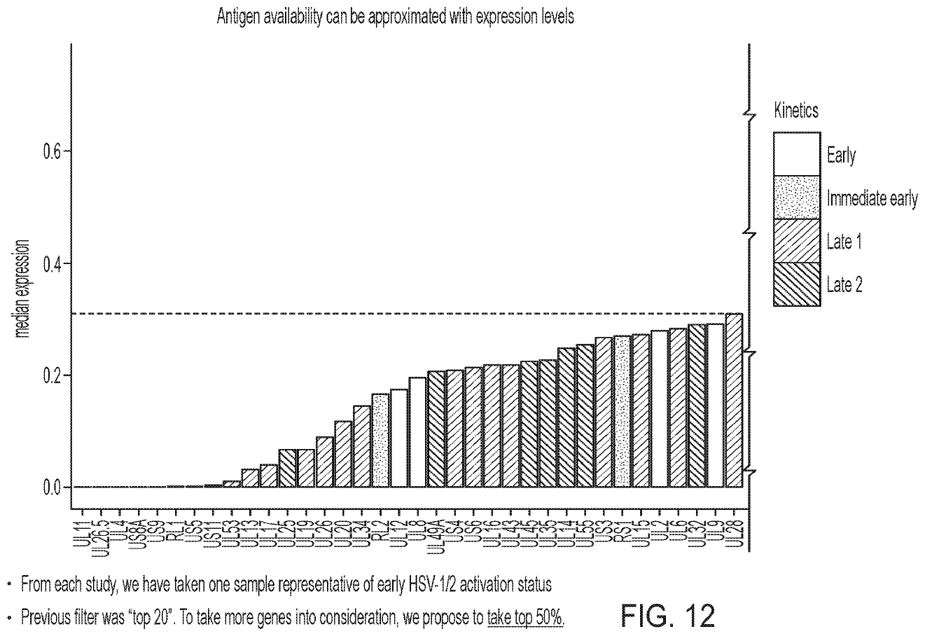

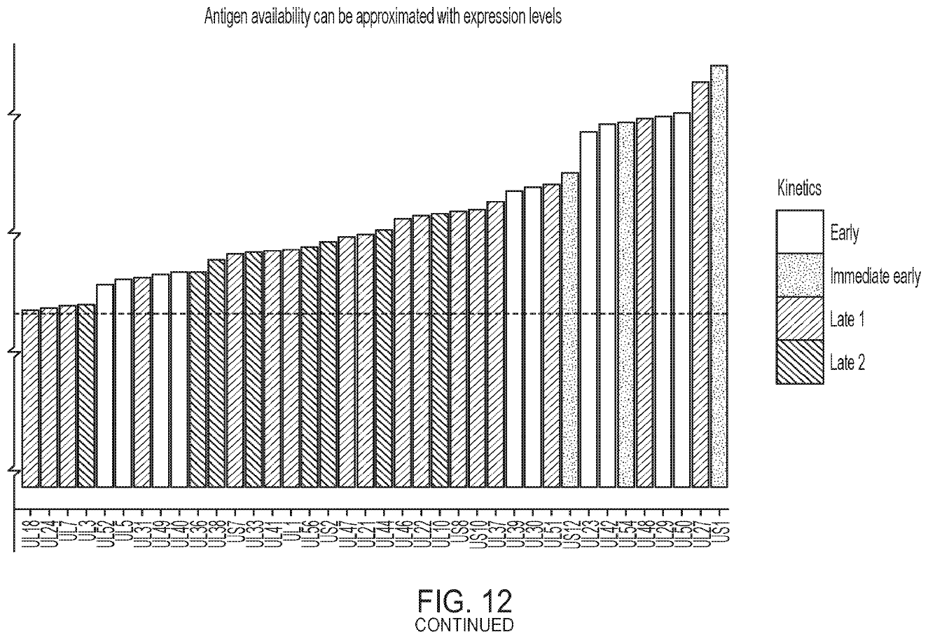

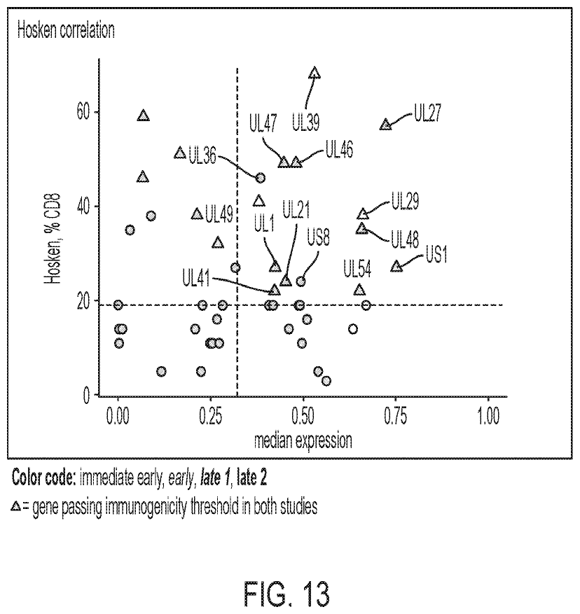

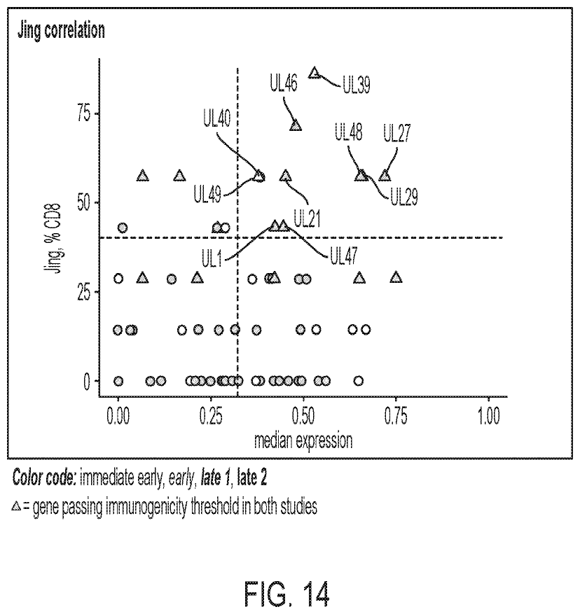

As described herein, to initiate infection, an HSV (HSV-1 or HSV-2) particle binds to the cell surface using the viral glycoproteins and fuses its envelope with the plasma membrane (see, e.g., FIG. 2, Step 1). After the fusion of membranes, the viral capsid and tegument proteins are internalized in the cytoplasm (see, e.g., FIG. 2, Step 2). Once in the cytoplasm, the viral capsid accumulates in the nucleus and releases viral DNA into the nucleus (see, e.g., FIG. 2, Step 3). HSV replicates by three rounds of transcription that yield: u (immediate early) proteins that mainly regulate viral replication; β (early) proteins that synthesise and package DNA; and γ (late) proteins, most of which are virion proteins (see, Whitley et. al., Lancet 2001 May 12; 357(9267); Taylor et. al., Front Biosci. 2002 Mar. 1; 7:d752-64; and Ibáñez et. al., Front Microbiol. 2018 Oct. 11; 9:2406; each of which is incorporated herein by reference in its entirety) (see, e.g., FIG. 2, Steps 4-6).

The HSV capsids are assembled within the nucleus of infected cells (see, e.g., FIG. 2, Step 7). Once the assembly of viral capsids has been completed in the nucleus, these particles will continue their maturation process in this same compartment through the acquisition of tegument proteins. After leaving the nucleus, additional tegument proteins will be added to the capsids. Meanwhile, the glycoproteins are translated and glycosylated in the endoplasmic reticulum and processed in the trans-Golgi network (TGN) and then directed to multivesicular bodies (see, e.g., FIG. 2, Step 8). Then, they are exported to the plasma membrane glycoproteins within early endosomes (see, e.g., FIG. 2, Step 9). Viral capsids in the cytoplasm will then fuse with HSV-glycoprotein-containing endosomes to form infectious virions within vesicles (see, e.g., FIG. 2, Steps 10-12).

HSV (HSV-1 or HSV-2) are able to establish a latent infection. After primary infection, HSV either replicates productively in epithelial cells or enters sensory neuron axons and moves to the neuronal cell nucleus. There, the viral DNA remains as circular, extra-chromosomal DNA, and does not possess any lytic gene expression; however, latency associated transcripts are expressed and then spliced to produce mRNA. This general transcriptional silence may allow the virus to remain hidden in the cell by avoiding immune surveillance. In some aspects, provided herein are technologies (e.g., compositions and methods) for augmenting, inducing, promoting, enhancing and/or improving an immune response against HSV (e.g., HSV-1 and/or HSV-2) or a component thereof (e.g., a protein or fragment thereof). In some embodiments, technologies provided herein are designed to augment, induce, promote, enhance and/or improve immunological memory against HSV or a component thereof (e.g., a protein or fragment thereof). In some embodiments, technologies described herein are designed to act as an immunological boost to a primary vaccine, such as a vaccine directed to an antigens and/or epitopes of HSV (e.g., HSV-1 and/or HSV-2).

The virus remains in this state for the lifetime of the host, or until the proper signals reactivate the virus and new progeny are generated. Progeny virus then travel through the neuron axis to the site of the primary infection to re-initiate a lytic replication cycle.

### B. HSV Genome

The genome of HSV-1 and the genome of HSV-2 are both approximately 150 kb long of double-stranded DNA, varying slightly between subtypes and strains. The genome encodes more than 80 genes and has high GC contents: 67 and 69% for HSV-1 and HSV-2, respectively (see, Whitley et. al., Lancet 2001 May 12; 357(9267); Taylor et. al., Front Biosci. 2002 Mar. 1; 7:d752-64; and Jiao et. al., Microbiol Resour Announc. 2019 September; 8(39): e00993-19, which is incorporated herein by reference in its entirety).

The genome is organized as unique long region (UL) and a unique short region (US). The UL is typically bounded by terminal long (TRL) and internal long (IRL) repeats. The US is typically bounded by terminal short (IRS) and internal short (TRS) repeats. The genes found in the unique regions are present in the genome as a single copy, but genes that are encoded in the repeat regions are present in the genome in two copies (see, Whitley et. al., Lancet 2001 May 12; 357(9267); Taylor et. al., Front Biosci. 2002 Mar. 1; 7:d752-64; and Jiao et. al., Microbiol Resour Announc. 2019 September; 8(39): e00993-19, which is incorporated herein by reference in its entirety).

HSV contains three origins of replication within the genome that are named depending upon their location in either the Long (oriL) or Short (oriS) region of the genome. OriL is found as a single copy in the UL segment, but oriS is located in the repeat region of the Short segment; thus, it is present in the genome in two copies. Both oriL and oriS are palindromic sequences consisting of an AT-rich center region flanked by inverted repeats that contain multiple binding sites of varying affinity for the viral origin binding polypeptide (UL9). Either oriL or one of the oriS sequences is sufficient for viral replication (see, Whitley et. al., Lancet 2001 May 12; 357(9267); Taylor et. al., Front Biosci. 2002 Mar. 1; 7:d752-64; and Jiao et. al., Microbiol Resour Announc. 2019 September; 8(39): e00993-19, which is incorporated herein by reference in its entirety).

The viral genome also contains signals that orchestrate proper processing of the newly synthesized genomes for packaging into pre-formed capsids. Progeny genomes are generated in long concatemers that require cleavage into unit-length monomers. For this purpose, the viral genome contains two DNA sequence elements, pac1 and pac2, that ensure proper cleavage and packaging of unit-length progeny genomes. These elements are located within the direct repeats (DR) found within the inverted repeat regions at the ends of the viral genome (see, Whitley et. al., Lancet 2001 May 12; 357(9267); Taylor et. al., Front Biosci. 2002 Mar. 1; 7:d752-64; and Jiao et. al., Microbiol Resour Announc. 2019 September; 8(39): e00993-19, which is incorporated herein by reference in its entirety).

### C. Certain HSV Proteins

### ICP0

Infected cell protein 0 (ICP0) of herpes simplex virus 1 (HSV-1) is an a (immediate-early) protein of herpes simplex virus 1, and is capable of activating HSV-1 gene expression, disrupt nuclear domain (ND) 10 structures, mediate the degradation of cellular proteins, and evade the host cell's intrinsic and innate antiviral defenses (see., Smith et. al., Future Virol. 2011 April; 6(4): 421-429).

### ICP22

Infected cell protein 22 (ICP22) is expressed from an immediate-early (IE) gene during the replication cycle of HSV-1 and HSV-2. ICP22 can generally regulate viral and host gene transcription by changing the phosphorylation status of host RNA polymerase II (RNA pol II) and can also facilitate the nuclear egress complex (NEC) accurately locate to the nuclear membrane to promote nuclear budding (see, Wu et. al., Front Microbiol. 2021 Jun. 7; 12:668461).

### VP16

The UL48 gene encodes VP16 or alpha-gene-transactivating factor (α-TIF). VP16 is an important transactivator that can activate the transcription of viral immediate-early genes, and in the late stage of viral replication. Additionally, VP16, as a tegument, is involved in viral assembly (see Fan, et. al., Front Microbiol. 2020; 11: 1910).

In the early stage of viral infection, VP16 released by invading virions binds to the immediate-early (IE) gene promoter to stimulate the transcription of IE genes as a transactivating factor that acts specifically on IE genes (see Fan, et. al., Front Microbiol. 2020; 11: 1910). In the late stage, VP16 assembles into the tegument to participate in the assembly of virions and promote their maturation (see Fan, et. al., Front Microbiol. 2020; 11: 1910).

### Glycoproteins

In order to replicate, enveloped HSV must be able to fuse with the membrane of a living cell and deliver their genetic material into its cytoplasm. The HSV viral envelope surrounding the tegument has at least 12 different glycoproteins (gB-gN) on their surface. The glycoproteins may exist as heterodimers (gH/gL and gE/gI) with most existing as monomers. HSV gC, gB, gD, gH, and gL are involved in the process of viral cell entry. Initial attachment is mediated by gC, followed by gD. Then gH/gL pull the virus and the cell membrane together, and then gB triggers the membrane fusion. (Reske et. al., Rev Med Virol. May-June 2007; and Arii et. al., Adv Exp Med Biol. 2018; 1045:3-21).

The present disclosure provides HSV glycoprotein (e.g., gB, gC, gD, gE, gG, gH, gI, and/or gL) antigens and antigenic fragments thereof can be useful in preventing or treating HSV, e.g., in HSV antigen constructs and/or HSV compositions (e.g., immunogenic compositions, e.g., vaccines) as further disclosed herein.

### Glycoprotein C (gC)

Mature HSV glycoprotein C (gC) is a 56 kDa protein that plays a role in initial cell attachment. Glycoprotein C is a type I membrane glycoprotein and is considered a significant attachment protein and principle viral ligand for binding heparin sulfate proteoglycans (HSPGs) on a cell surface. This binding can occur by gC interaction with HSPG rich regions found on F-actin rich membrane protrusions referred to as filopodia.

Glycoprotein C has also been shown to be involved in regulation of cell entry and infection by increasing pH threshold for acid-induced conformational changes of gB. Low pH induces reversible conformational changes to gB domains I and V, the functional region containing hydrophobic loops important in cell fusion. By positively regulating low-pH-induced conformational changes of gB, gC can enhance HSV's ability to invade cell types, like epithelial cells, that require a low-pH mechanism for invasion.

Glycoprotein C has also been shown to play a role in immune evasion, in addition to its role in attachment. Glycoprotein C is a target for lymphocyte cytotoxicity in certain cell types and is able to bind complement component C3b to inhibit compliment activation. Furthermore, neutralizing epitopes that exist on other HSV glycoproteins, like gB, can be protected by gC, preventing immune responses from blocking fusion.

### Glycoprotein D (gD)

HSV glycoprotein D (gD) is a 46 kDA type I membrane glycoprotein. The N-terminal ectodomain is comprised of 316 amino acids. Glycoprotein D facilitates invasion by interacting with several cell surface receptors, including herpesvirus entry mediator (HVEM), nectin-1 or nectin-2, and heparin sulfate that contain specific modifications. These cellular receptors do not function as co-receptors, as each glycoprotein interaction with a cell's receptor occurs independently of each other. Binding of gD to one of these cellular receptors causes a conformational change that converts gD's auto-inhibitory closed state into an active state that transmits one of two signals believed to be required for gH/gL complex activation. HVEM, the first gD receptor identified, belongs to the tumor necrosis factor receptor family and is commonly found on T cells, B cells, dendritic cells, natural killer cells, macrophages, as well as non-immune cell types like neurons and epithelial cells. Within the N-terminus of gD, there is a 37 residue hairpin structure that forms the entire site for binding to HVEM. Specifically, residues 1-32 of gD's N-terminal domain bind HVEM's cysteine-rich domain 1. When not in contact with HVEM, this N-terminal extension adopts an extended and flexible conformation.

Clinical strains of HSV use nectin-1 for cell entry; however, several mutant strains of HSV utilize nectin-2. Furthermore, heparin sulfate is utilized by HSV-1 but not HSV-2. Glycoprotein D interaction with net-1 has been shown to be essential in some cell types such as neurons, even when other receptors are present on a cell surface. Glycoprotein H (gH)/Glycoprotein L (gL) Complex

Glycoprotein H (gH) is an essential 56 kD protein that exists as a heterodimeric complex with 25 kDa glycoprotein L (gL) (complex referred to herein as gH/gL). The gH/gL complex is required for cell fusion and entry. gH/gL does not share any structural similarities with documented fusion proteins and likely does not function as a cofusogen with gB. Instead, gH/gL may act as a regulator of fusion and important component in stabilizing contact between HSV and a cell. Glycoprotein H receives a signal from gD through its H1 domain, and transmits this signal to membrane proximal H3 domain, which in turn propagates that signal to gH's cytoplasmic tail. Once gH's cytoplasmic tail receives this signal, it releases strain on the pre-fusion conformation of gB, which favors attachment of gB's fusion loop to a cell surface, promoting gB mediated membrane fusion. Mutations in gH's C-terminal tail have been shown to reduce fusion activity. Furthermore, antibody responses directed towards gH have been shown capable of inhibiting fusion processes mediated by gB-gH-gL. In addition to this essential role, gH contains an arginylglycylaspartic acid (RGD) motif that can bind integrin receptors found on cells. Interaction of gH with integrin is believed to trigger intracellular signals which facilitate capsid transport.

### Glycoprotein B (gB)

Glycoprotein B is a protein that has an apparent molecular weight of approximately 95-100 kDa and consists of an extended rod or spike-like ectodomain, a hydrophobic membrane proximal region (MPR), a transmembrane region (TMR), and a C-terminal domain (CTD). The ectodomain is well characterized to actively participate in fusion, while MPR, TMR, and CTD can play roles in regulation fusion. Glycoprotein B is a class III fusogen. Glycoprotein B ectodomain architecture shares conformational similarity with fusogens from viruses not belonging to the herpesvirdae family. Glycoprotein B is activated through its interaction with gH/gL, but HSV cannot fuse with a target cell through activation of gB alone and requires gB interaction to specific receptors for fusion to be completed. A well-known receptor target of gB is cell-surface heparin sulfate, an interaction that is not essential for HSV fusion, but is known to promote viral adhesion to a cell surface. Glycoprotein B can also interact with paired immunoglobulin-like type 2 receptor, most commonly found on monocytes, macrophages, and dendritic cells.

HSV gB exists in two forms, a pre-fusion and post fusion form. Several changes in the pre-fusion form of gB are thought to lead to its active and post-fusion state. The first change occurs at domain V or at MPR, which allows fusion loops to point towards a cell membrane and away from a viral membrane. This change can produce a compacting intermediate conformation 1 that does not yet attach to a cell membrane surface. The next change occurs at domain III and involves gB adopting an extended intermediate conformation 2 that allows its fusion loop to attach to a cell membrane surface. Lastly, changes in domain V convert gB to its post-fusion conformation that favors membrane fusion.

The post-fusion form of HSV-1 gB has an ectodomain that exists as three protomers that interact to produce a rod-like trimeric structure. Each promoter is comprised of five distinct domains with linker regions that individually form a hairpin shape. Each domain of an individual protomer interacts with the same domain of an adjacent protomer to form the described trimeric structure. Domain I houses an important fusion loop and is commonly referred to as the fusion domain. Domain II facilitates interactions with gH/gL and is referred to as the gH/gL domain. Domain III is comprised of alpha helices that help form the trimeric coil-coil central core of this protein. Domain IV is referred to as the crown domain and sits on top of the post-fusion form; it is believed to bind with cellular receptors. Antibodies that bind to the crown domain can disrupt gB binding to cellular receptors. Domain V consists of a long extension and connects protomers together.

Glycoprotein E and Glycoprotein I (gE/gI)

Glycoprotein E is approximately 53 kDa and Glycoprotein I is approximately 141 kDa. Both proteins interact to form a heterodimeric complex (complex referred to herein as gE/gI) that plays a role in cell-to-cell spread and virus induced fusion. The gE/gI complex, unlike gB, gD, and gH/gL, is not required for fusion and entrance into a cell, but is important for cell-to-cell spread. Disruption of gE/gI formation has effects on HSV proliferation, as this virus relies on cell-to-cell spread for its lytic cycle. The mechanism in which gE/gI facilitate cell-to-cell spread is thought to be reliant on several tegument polypeptides. Cooperation of tegument polypeptides, UL11, UL16, and UL21 may play a role in processing, transport, and biological activity of gE.

### Glycoprotein G

Glycoprotein G (gG) from both HSV-1 (gG1) and HSV-1 (gG2) is the first viral chemokine-binding protein shown to potentiate chemokine function of a cell. Glycoprotein G varies in size significantly between HSV-1 and HSV-2, with a 76 kDa and 43 kDa size, respectively. Glycoprotein G is unique in that its soluble form (SgG2) can have immune modulatory capacity through its extracellular activity. Once extracellular, SgG2 binds chemokines through the glycosaminoglycan (GAG)-binding domain of a chemokine without interfering with chemokine's G protein coupled receptors (GPCRs) binding site. SgG2's interaction with GAG containing proteins allows initiation of lipid raft formation and accumulation, which produces a clustering of chemokine receptors into this micro domain. Clustering of chemokine receptors, in turn, increases local concentration of chemokines on a host cell's extracellular surface and allows these chemokines to interact with GPCRs. This interaction likely leads to increased immune signaling responses and chemokine stimulation. This combination of receptor relocalization and presentation of the chemokine complex with SgG2 provides a molecular rationale for enhancement of chemokine function during HSV infection. This immune modulation is in contrast to what is seen in other viruses that inhibit chemokine function, as in this case, chemokine function is potentiated by SgGs. Without being bound to any particular theory, it is thought that an overall manipulation of endogenous immune signaling may be overall favorable to HSV.

### ICP47

Infected cell protein 47 (ICP47) encoded by gene US12, is a polymorphous protein and could block RNA splicing in early infection, and then, shuttle viral mRNA from nucleus to cytoplasm in late infection. ICP47 directly binds antigen-dependent transporter (TAP), limiting antigen trafficking, leading to the occurrence of empty MHC-I (Cheng et. al., Virol J. 2020 Jul. 10; 17(1):101). The binding of ICP47 to TAP stabilizes the inward conformation, therefore blocking the translocation pathway points to the endoplasmic reticulum (ER) cavity. By blocking the entry of viral antigens into ER, HSV could avoid the attack of cytotoxic T lymphocytes, which may lead to immune escape of HSV and establish lifelong infection in the host cells (Cheng et. al., Virol J. 2020 Jul. 10; 17(1):101).

### VHS

The virion-host shutoff (VHS) protein is viral protein synthesized with late kinetics and packaged into mature virion particles. Functionally, VHS is a viral RNase that preferentially degrade both host and viral mRNA species. VHS has been reported to interfere with dendritic cells (DC) activation during both productive and nonproductive HSV infection (Cotter et. al., J Virol. 2011 December; 85(23): 12662-12672.).

### US3

All members of the Alphaherpesvirinae subfamily encode a serine/threonine kinase, designated US3. US3 is a significant virulence factor for herpes simplex virus type 1 (HSV-1), and is a multifunctional polypeptide that plays various roles in the viral life cycle by phosphorylating a number of viral and cellular substrates (Kato et. al., Adv Exp Med Biol. 2018; 1045:45-62.).

### D. HSV Vaccines

Several HSV vaccines, mainly targeting HSV-2 and primarily focused on the generation of neutralizing antibodies (nAbs) targeting the viral envelope glycoprotein D as the correlate of immune protection, have been developed and evaluated in human clinical trial, see table 1 below. Despite these vaccines exhibiting protection against HSV in preclinical studies and in some cases Phase 2 studies, none of these vaccines has demonstrated sufficient efficacy for further development or commercialization.

The present disclosure provides an insight that many prior strategies for developing pharmaceutical compositions (e.g., immunogenic compositions, e.g., vaccines) for treatment of and/or protection from HSV infection have focused primarily, or even almost exclusively, on development of neutralizing antibodies that target surface glycoproteins. The present disclosure identifies a problem with such strategies including, for example, that they may fail to appreciate value or even criticality of ensuring that an induced immune response includes significant T cell activity (in some embodiments, CD4 T cell activity, in some embodiments CD8 T cell activity, in some embodiments, both). In some embodiments, pharmaceutical compositions (e.g., immunogenic compositions, e.g., vaccines) that comprise or deliver CD4 and CD8 epitope(s) of one or more HSV antigens (e.g., HSV-1 antigens, HSV-2 antigens, or a combination thereof), e.g., in addition to one or more B cell antigens and/or epitopes may be used in treatment of and/or protection from HSV infection.

TABLE 1
Certain HSV Vaccines Under Clinical Development

| | | | Immune | |
|---|---|---|---|---|
| Name | Platform | Antigens | Response | Clinical results |
| gB/gD/MF59 | Subunit | gD and gB | Neutralzing | Phase 3 |
| (Chiron) | MF59 adjuvant | | antibodies (Abs) | Prophylaxis |
| | | | | 9% (95% Cl: −29%, |
| | | | | 36%) |
| gD/AS04 | Subunit | gD | Neutralizing Abs | Phase 3 |
| (GSK) | AS04 adjuvant | | CD4⁺ T cells | Prophylaxis |
| | | | | 20% (95% CI: −29%, |
| | | | | 50%) |
| gH-null | Single-cycle | Multiple | Neutralizing Abs | Phase 3 |
| (Cantab | live | | | therapeutic |
| Pharmaceutical) | | | | No difference |
| | | | | in recurrences |
| | | | | or shedding |
| HSV529 | Replication- | Multiple | Neutralizing Abs | Phase 1 |
| (Sanofi-Pasteur) | defective | | CD4⁺, | Safe but |
| | | | CD8⁺ T cells | immunogenic |
| | | | | only in doubly |
| | | | | seronegatives |
| Gen-003 | Subunit | gD, ICP4 | Neutralizing Abs | Phase 2 |
| (Genocea) | Saponin | | Cytolytic T cells | therapeutic |
| | adjuvant | | | Dose variable |
| | | | | decrease |
| | | | | shedding |
| Herp V | Heat shock | 32 HSV | Cytotoxic T | Phase 2 |
| (Agenus) | protein 70-HSV | peptides | cells | therapeutic |
| | peptides QS-21 | (gD, | | 17% reduction |
| | adjuvant | additional | | in shedding |
| | | envelope, | | frequency |
| | | tegument, and | | |
| | | other) | | |
| Vaxfectin | DNA | gD, UL46/47 | Neutralizing Abs | Phase 1/2 |
| (Vical) | Vaxfectin | | | therapeutic |
| | adjuvant | | | Failed to reduce |
| | | | | shedding |
| COR-1 | Codon | Codon | gD-specific Abs | Phase 2 |
| (Admedus) | optimized DNA | optimized | Cytotoxic T | therapeutic |
| | | gD2 and | cells | No difference |
| | | ubiquitinfused | | in recurrences |
| | | truncated gD2 | | |
| | | to target the | | |
| | | antigen to the | | |
| | | proteasome | | |
| ΔNLS | Deleted in ICP0 | Multiple | Neutralizing Abs | Phase 1 non- |
| (Rational | Replication | targets | gD-specific Abs | FDA |
| Vaccines) | competent | | | Approved |
| | Attenuated for | | | therapeutic |
| | latency | | | study |
| | | | | Self-reported |
| | | | | reduction in |
| | | | | recurrences |

### E. Anti-Viral Treatments for HSV

The present disclosure provides the recognition that constructs and/or compositions described herein may be administered as part of regimen with other therapeutic agents. The present disclosure also recognizes that subjects that are administered constructs and/or compositions described herein may have previously been administered other therapeutic agents.

In some embodiments, for example, a subject may be receiving or had previously received an anti-viral agent for HSV. In some embodiments, an anti-viral agent can be administered to treat HSV-1 or HSV-2 infection or recurrent episodes. In some embodiments, an anti-viral agent is or comprises acyclovir, valacyclovir, famciclovir, or a combination thereof. Table 2 below provides certain information about select anti-viral agents.

TABLE 2
Antiviral Drugs for Treating HSV

| | | | |
|---|---|---|---|
| | | | Approximate |
| | | | price for |
| Medica- | | Most common adverse | complete |
| tion | Dosage | effects | dosing¹ |

Oral formulations:

| | | | |
|---|---|---|---|
| acyclovir | 200-400 mg 5x/day; | malaise, headache, | 35-800 mg |
| | 5-365 days | nausea, vomiting | tabs; $70 |
| valacyclovir | 1 g 3x/day; 7 days | headache, nausea, | 21-1 g |
| | | abdominal pain | tabs; $220 |
| famciclovir | 250-1500 mg 2- | headache, nausea, | 21-500 mg |
| | 3x/day; 1-30 days | fatigue, diarrhea | tabs; $184 |

Intravenous formulations:

| | | |
|---|---|---|
| acyclovir | 10-15 mg/kg every | phlebitis, acute renal |
| | 8 h; 7 days | failure, nausea, |
| | | vomiting, rash |
| foscarnet | 40-60 mg/kg/day, | fever, headache, renal |
| | 14-21 days | dysfunction, |
| | | electrolyte |
| | | abnormalities, nausea, |
| | | vomiting diarrhea, |
| | | anemia, |
| | | granulocytopenia |

Topical application

### II. Constructs

### A. HSV Antigens

The present disclosure provides HSV (e.g., HSV-1, HSV-2 or both) antigens and antigenic fragments thereof can be useful in preventing or treating HSV, e.g., in HSV antigen constructs and/or HSV compositions (e.g., immunogenic compositions, e.g., vaccines) as further disclosed herein.

In some embodiments, HSV antigenic fragments can be useful in a HSV T cell antigen construct. In some embodiments, a HSV antigen (e.g., a full length HSV antigen) can be useful in a HSV glycoprotein construct.

In some embodiments, a polyribonucleotide encodes one or more HSV antigens or antigenic fragments thereof. In some embodiments, a polyribonucleotide encodes a HSV glycoprotein construct.

A number of HSV antigens are known. The present disclosure provides a polyribonucleotide that encodes an HSV antigen as described herein or an antigenic fragment thereof. An overview of exemplary amino acids sequences of certain HSV (HSV-1, HSV-2, or both) proteins are provided in Tables 3-5 below. Exemplary amino acid sequences of certain HSV (HSV-1, HSV-2, or both) proteins are provided in Table 6 below.

TABLE 3
Exemplary antigens for HSV

| | | Exemplary Amino Acid Sequences | | | |
|---|---|---|---|---|---|
| | | (SEQ ID NOs) | | | |
| Antigen | Kinetics | HG52 | 333 | MS | G |
| UL1 | Late 1 | 1 | 2 | 3 | |
| UL21 | Late 1 | 4 | 5 | 6 | |
| UL27 [HSV-2 gB] | Late 1 | 7 | 8 | 9 | 74 |
| UL29 | Early | 10 | 11 | 12 | |
| UL39 | Early | 13 | 14 | 15 | |
| UL40 | Early | 16 | 17 | 18 | |
| UL46 | Late 1 | 19 | 20 | 21 | |
| UL47 | Late 1 | 22 | 23 | 24 | |
| UL48 | Late 1 | 25 | 26 | 27 | |
| UL49 | Early | 28 | 29 | 30 | |
| RS1 [ICP4] | IE | 31 | 32 | 33 | |
| RL2 [ICP0] | IE | 34 | 35 | 36 | |
| UL5 | Early | 37 | 38 | 39 | |
| UL9 | Early | 40 | 41 | 42 | |
| UL19 | Late 1 | 43 | 44 | 45 | |
| UL25 | Late 2 | 46 | 47 | 48 | |
| UL30 | Early | 49 | 50 | 51 | |
| UL52 | Early | 52 | 53 | 54 | |
| UL54 | IE | 55 | 56 | 57 | |
| US1 [HSV-2 gL] | | 58 | 59 | 60 | 61 |
| US7 [HSV-2 gI] | | 62 | 63 | 64 | 65 |
| US8 [HSV-2 gE] | | 66 | 67 | 68 | 69 |
| UL22 [HSV-2 gH] | | 70 | 71 | 72 | 73 |
| US6 [HSV-2 gD] | | | 221 | | |
| UL44 [HSV-2 gC] | | | 222 | | |

TABLE 4
Exemplary antigens selected from systematic
analysis of source data

| | | Exemplary Amino Acid Sequences | | | |
|---|---|---|---|---|---|
| | | (SEQ ID NOs) | | | |
| Antigen | Kinetics | HG52 | 333 | MS | G |
| UL1 | Late 1 | 1 | 2 | 3 | |
| UL21 | Late 1 | 4 | 5 | 6 | |
| UL27 [HSV-2 gB] | Late 1 | 7 | 8 | 9 | 74 |
| UL29 | Early | 10 | 11 | 12 | |
| UL39 | Early | 13 | 14 | 15 | |
| UL40 | Early | 16 | 17 | 18 | |
| UL46 | Late 1 | 19 | 20 | 21 | |
| UL47 | Late 1 | 22 | 23 | 24 | |
| UL48 | Late 1 | 25 | 26 | 27 | |
| UL49 | Early | 28 | 29 | 30 | |

TABLE 5
Exemplary antigens

| | | Exemplary Amino Acid Sequences | | | |
|---|---|---|---|---|---|
| | | (SEQ ID NOs) | | | |
| Antigen | Kinetics | HG52 | 333 | MS | G |
| RS1 [ICP4] | IE | 31 | 32 | 33 | |
| RL2 [ICP0] | IE | 34 | 35 | 36 | |
| UL5 | Early | 37 | 38 | 39 | |
| UL9 | Early | 40 | 41 | 42 | |
| UL19 | Late 1 | 43 | 44 | 45 | |
| UL25 | Late 2 | 46 | 47 | 48 | |
| UL30 | Early | 49 | 50 | 51 | |
| UL52 | Early | 52 | 53 | 54 | |
| UL54 | IE | 55 | 56 | 57 | |
| US1 [HSV-2 gL] | | 58 | 59 | 60 | 61 |
| US7 [HSV-2 gI] | | 62 | 63 | 64 | 65 |
| US8 [HSV-2 gE] | | 66 | 67 | 68 | 69 |
| UL22 [HSV-2 gH] | | 70 | 71 | 72 | 73 |
| US6 [HSV-2 gD] | | | | | |
| UL44 [HSV-2 gC] | | | | | |

TABLE 6
Exemplary amino acid antigen sequences

| SEQ | | | |
|---|---|---|---|
| ID | | | |
| NO | Antigen | Strain | Amino Acid Sequence |
| 1 | UL1 | HG52 | MGFVCLFGLVVMGAWGAWGGSQATEYVLRSVIAKEVGDIL |
| | | | RVPCMRTPADDVSWRYEAPSVIDYARIDGIFLRYHCPGLD |
| | | | TFLWDRHAQRAYLVNPFLFAAGFLEDLSHSVFPADTQETT |
| | | | TRRALYKEIRDALGSRKQAVSHAPVRAGCVNFDYSRTRRC |
| | | | VGRRDLRPANTTSTWEPPVSSDDEASSQSKPLATQPPVLA |
| | | | LSNAPPRRVSPTRGRRRHTRLRRN |
| 2 | UL1 | 333 | MGFVCLFGLVVMGAWGAWGGSQATEYVLRSVIAKEVGDIL |
| | | | RVPCMRTPADDVSWRYEAPSVIDYARIDGIFLRYHCPGLD |
| | | | TFLWDRHAQRAYLVNPFLFAAGFLEDLSHSVFPADTQETT |
| | | | TRRALYKEIRDALGSRKQAVSHAPVRAGCVNFDYSRTRRC |
| | | | VGRRDLRPANTTSTWEPPVSSDDEASSQSKPLATQPPVLA |
| | | | LSNAPPRRVSPTRGRRRHTRLRRN |
| 3 | UL1 | MS | MGFVCLFGLVVMGAWGAWGGSQATEYVLRSVIAKEVGDIL |
| | | | RVPCMRTPADDVSWRYEAPSVIDYARIDGIFLRYHCPGLD |
| | | | TFLWDRHAQRAYLVNPFLFAAGFLEDLSHSVFPADTQETT |
| | | | TRRALYKEIRDALGSRKQAVSHAPVRAGCVNFDYSRTRRC |
| | | | VGRRDLRPANTTSTWEPPVSSDDEASSQSKPLATQPPVLA |
| | | | LSNAPPRRVSPTRGRRRHTRLRRN |
| 4 | UL21 | HG52 | MELSYATTLHHRDVVFYVTADRNRAYFVCGGSVYSVGRPR |
| | | | DSQPGEIAKFGLVVRGTGPKDRMVANYVRSELRQRGLRDV |
| | | | RPVGEDEVFLDSVCLLNPNVSSERDVINTNDVEVLDECLA |
| | | | EYCTSLRTSPGVLVTGVRVRARDRVIELFEHPAIVNISSR |
| | | | FAYTPSPYVFALAQAHLPRLPSSLEPLVSGLFDGIPAPRQ |
| | | | PLDARDRRTDVVITGTRAPRPMAGTGAGGAGAKRATVSEF |
| | | | VQVKHIDRVVSPSVSSAPPPSAPDASLPPPGLQEAAPPGP |
| | | | PLRELWWVFYAGDRALEEPHAESGLTREEVRAVHGFREQA |
| | | | WKLFGSVGAPRAFLGAALALSPTQKLAVYYYLIHRERRMS |
| | | | PFPALVRLVGRYIQRHGLYVPAPDEPTLADAMNGLFRDAL |
| | | | AAGTVAEQLLMFDLLPPKDVPVGSDARADSAALLRFVDSQ |
| | | | RLTPGGSVSPEHVMYLGAFLGVLYAGHGRLAAATHTARLT |
| | | | GVTSLVLTVGDVDRMSAFDRGPAGAAGRTRTAGYLDALLT |
| | | | VCLARAQHGQSV |
| 5 | UL21 | 333 | MELSYATTLHHRDVVFYVTADRNRAYFVCGGSVYSVGRPR |
| | | | DSQPGEIAKFGLVVRGTGPKDRMVANYVRSELRQRGLRDV |
| | | | RPVGEDEVFLDSVCLLNPNVSSERDVINTNDVEVLDECLA |
| | | | EYCTSLRTSPGVLVTGVRVRARDRVIELFEHPAIVNISSR |
| | | | FAYTPSPYVFALAQAHLPRLPSSLEPLVSGLFDGIPAPRQ |
| | | | PLDARDRRTDVVITGTRAPRPMAGTGAGGAGAKRATVSEF |
| | | | VQVKHIDRVVSPSVSSAPPPSAPDASLPPPGLQEAAPPGP |
| | | | PLRELWWVFYAGDRALEEPHAESGLTREEVRAVHGFREQA |
| | | | WKLFGSVGAPRAFLGAALALSPTQKLAVYYYLIHRERRMS |
| | | | PFPALVRLVGRYIQRHGLYVPAPDEPTLADAMNGLFRDAL |
| | | | AAGTVAEQLLMFDLLPPKDVPVGSDARADSAALLRFVDSQ |
| | | | RLTPGGSVSPEHVMYLGAFLGVLYAGHGRLAAATHTARLT |
| | | | GVTSLVLTVGDVDRMSAFDRGPAGAAGRTRTAGYLDALLT |
| | | | VCLARAQHGQSV |
| 6 | UL21 | MS | MELSYATTLHHRDVVFYVTADRNRAYFVCGGSVYSVGRPR |
| | | | ASQPGEIAKFGLVVRGTGPKDRMVANYVRSELRQRGLRDV |
| | | | RPVGEDEVFLDSVCLLNPNVSSERDVINTNDVEVLDECLA |
| | | | EYCTSLRTSPGVLVTGVRVRARDRVIELFEHPAIVNISSR |
| | | | FVYTPSPYVFALAQAHLPRLPSSLEPLVSGLFDGIPAPRQ |
| | | | PLDARDRRTDVVITGTRAPRPMAGTGAGGAGAKRATVSEF |
| | | | VQVKHIDRVVSPSVSSAPPPSAPDASLPPPGLQEAAPPGP |
| | | | PLRELWWVFYAGDRALEEPHAESGLTREEVRAVHGFREQA |
| | | | WKLFGSVGAPRAFLGAALALSPTQKLAVYYYLIHRERRMS |
| | | | PFPALVRLVGRYIQRHGLYVPAPDEPTLADAMNGLFRDAL |
| | | | AAGTVAEQLLMFDLLPPKDVPVGSDARADSAALLRFVDSQ |
| | | | RLTPGGSVSPEHVMYLGAFLGVLYAGHGRLAAATHTARLT |
| | | | GVTSLVLTVGDVDRMSAFDRGPAGAAGRTRTAGYLDALLT |
| | | | VCLARAQHGQSV |
| 7 | UL27 | HG52 | MRGGGLICALVVGALVAAVASAAPAAPAAPRASGGVAATV |
| | [HSV-2gB] | | AANGGPASRPPPVPSPATTKARKRKTKKPPKRPEATPPPD |
| | | | ANATVAAGHATLRAHLREIKVENADAQFYVCPPPTGATVV |
| | | | QFEQPRRCPTRPEGQNYTEGIAVVFKENIAPYKFKATMYY |
| | | | KDVTVSQVWFGHRYSQFMGIFEDRAPVPFEEVIDKINTKG |
| | | | VCRSTAKYVRNNMETTAFHRDDHETDMELKPAKVATRTSR |
| | | | GWHTTDLKYNPSRVEAFHRYGTTVNCIVEEVDARSVYPYD |
| | | | EFVLATGDFVYMSPFYGYREGSHTEHTSYAADRFKQVDGF |
| | | | YARDLTTKARATSPTTRNLLTTPKFTVAWDWVPKRPAVCT |
| | | | MTKWQEVDEMLRAEYGGSFRFSSDAISTTFTTNLTEYSLS |
| | | | RVDLGDCIGRDAREAIDRMFARKYNATHIKVGQPQYYLAT |
| | | | GGFLIAYQPLLSNTLAELYVREYMREQDRKPRNATPAPLR |
| | | | EAPSANASVERIKTTSSIEFARLQFTYNHIQRHVNDMLGR |
| | | | IAVAWCELQNHELTLWNEARKLNPNAIASATVGRRVSARM |
| | | | LGDVMAVSTCVPVAPDNVIVQNSMRVSSRPGTCYSRPLVS |
| | | | FRYEDQGPULIEGQLGENNELRLTRDALEPCTVGHRRYFI |
| | | | FGGGYVYFEEYAYSHQLSRADVTTVSTFIDLNITMLEDHE |
| | | | FVPLEVYTRHEIKDSGLLDYTEVQRRNQLHDLRFADIDTV |
| | | | IRADANAAMFAGLCAFFEGMGDLGRAVGKVVMGVVGGVVS |
| | | | AVSGVSSFMSNPFGALAVGLLVLAGLVAAFFAFRYVLQLQ |
| | | | RNPMKALYPLTTKELKTSDPGGVGGEGEEGAEGGGFDEAK |
| | | | LAEAREMIRYMALVSAMERTEHKARKKGTSALLSSKVTNM |
| | | | VLRKRNKARYSPLHNEDEAGDEDEL |
| 8 | UL27 | 333 | MRGGGLICALVVGALVAAVASAAPAAPAAPRASGGVAATV |
| | [HSV-2gB] | | AANGGPASRPPPVPSPATTKARKRKTKKPPKRPEATPPPD |
| | | | ANATVAAGHATLRAHLREIKVENADAQFYVCPPPTGATVV |
| | | | QFEQPRRCPTRPEGQNYTEGIAVVFKENIAPYKFKATMYY |
| | | | KDVTVSQVWFGHRYSQFMGIFEDRAPVPFEEVIDKINAKG |
| | | | VCRSTAKYVRNNMETTAFHRDDHETDMELKPAKVATRTSR |
| | | | GWHTTDLKYNPSRVEAFHRYGTTVNCIVEEVDARSVYPYD |
| | | | EFVLATGDFVYMSPFYGYREGSHTEHTSYAADRFKQVDGF |
| | | | YARDLTTKARATSPTTRNLLTTPKFTVAWDWVPKRPAVCT |
| | | | MTKWQEVDEMLRAEYGGSFRFSSDAISTTFTTNLTQYSLS |
| | | | RVDLGDCIGRDAREAIDRMFARKYNATHIKVGQPQYYLAT |
| | | | GGFLIAYQPLLSNTLAELYVREYMREQDRKPRNATPAPLR |
| | | | EAPSANASVERIKTTSSIEFARLQFTYNHIQRHVNDMLGR |
| | | | IAVAWCELQNHELTLWNEARKLNPNAIASATVGRRVSARM |
| | | | LGDVMAVSTCVPVAPDNVIVQNSMRVSSRPGTCYSRPLVS |
| | | | FRYEDQGPLIEGQLGENNELRLTRDALEPCTVGHRRYFIF |
| | | | GGGYVYFEEYAYSHQLSRADVTTVSTFIDLNITMLEDHEF |
| | | | VPLEVYTRHEIKDSGLLDYTEVQRRNQLHDLRFADIDTVI |
| | | | RADANAAMFAGLCAFFEGMGDLGRAVGKVVMGVVGGVVSA |
| | | | VSGVSSFMSNPFGALAVGLLVLAGLVAAFFAFRYVLQLQR |
| | | | NPMKALYPLTTKELKTSDPGGVGGEGEEGAEGGGFDEAKL |
| | | | AEAREMIRYMALVSAMERTEHKARKKGTSALLSSKVTNMV |
| | | | LRKRNKARYSPLHNEDEAGDEDEL |
| 9 | UL27 | MS | MRGGGLICALVVGALVAAVASAAPAAPRASGGVAATVAAN |
| | [HSV-2gB] | | GGPAPQPPPVPSPATTKARKRKTKKPPKRPEATPPPDANA |
| | | | TVAAGHATLRAHLREIKVENADAQFYVCPPPTGATVVQFE |
| | | | QPRRCPTRPEGQNYTEGIAVVFKENIAPYKFKATMYYKDV |
| | | | TVSQVWFGHRYSQFMGIFEDRAPVPFEEVIDKINAKGVCR |
| | | | STAKYVRNNMETTAFHRDDHETDMELKPAKVATRTSRGWH |
| | | | TTDLKYNPSRVEAFHRYGTTVNCIVEEVDARSVYPYDEFV |
| | | | LATGDFVYMSPFYGYREGSHTEHTSYAADRFKQVDGFYAR |
| | | | DLTTKAQATSPTTRNLLTTPKFTVAWDWVPKRPAVCTMTK |
| | | | WQEVDEMLRAEYGGSFRFSSDAISTTFTTNLTEYSLSRVD |
| | | | LGDCIGRDAREAIDRMFARKYNATHIKVGQPQYYLATGGF |
| | | | LIAYQPLLSNTLAELYVREYMREQDRKPRNATPAPLREAP |
| | | | SANASVERIKTTSSIEFARLQFTYNHIQRHVNDMLGRIAV |
| | | | AWCELQNHELTLWNEARKLNPNAIASATVGRRVSARMLGD |
| | | | VMAVSTCVPVAPDNVIVQNSMRVSSRPGTCYSRPLVSFRY |
| | | | EDQGPLIEGQLGENNELRLTRDALEPCTVGHRRYFIFGGG |
| | | | YVYFEEYAYSHQLSRADVTTVSTFIDLNITMLEDHEFVPL |
| | | | EVYTRHEIKDSGLLDYTEVQRRNQLHDLRFADIDTVIRAD |
| | | | ANAAMFAGLCAFFEGMGDLGRAVGKVVMGVVGGVVSAVSG |
| | | | VSSFMSNPFGALAVGLLVLAGLVAAFFAFRYVLQLQRNPM |
| | | | KALYPLTTKELKTSDPGGVGGEGEEGAEGGGFDEAKLAEA |
| | | | RQMIRYMALVSAMERTEHKARKKGTSALLSSKVTNMVLRK |
| | | | RNKARYSPLHNEDEAGDEDEL |
| 74 | UL27 | G | MRGGGLICALVVGALVAAVASAAPAAPRASGGVAATVAAN |
| | [HSV-2gB] | | GGPASRPPPVPSPATTRARKRKTKKPPERPEATPPPDANA |
| | | | TVAAGHATLRAHLREIKVENADAQFYVCPPPTGATVVQFE |
| | | | QPRRCPTRPEGQNYTEGIAVVFKENIAPYKFKATMYYKDV |
| | | | TVSQVWFGHRYSQFMGIFEDRAPVPFEEVIDKINAKGVCR |
| | | | STAKYVRNNMETTAFHRDDHETDMELKPAKVATRTSRGWH |
| | | | TTDLKYNPSRVEAFHRYGTTVNCIVEEVDARSVYPYDEFV |
| | | | LATGDFVYMSPFYGYREGSHTEHTSYAADRFKQVDGFYAR |
| | | | DLTTKARATSPTTRNLLTTPKFTVAWDWVPKRPAVCTMTK |
| | | | WQEVDEMLRAEYGGSFRFSSDAISTTFTTNLTQYSLSRVD |
| | | | LGDCIGRDAREAIDRMFARKYNATHIKVGQPQYYLATGGF |
| | | | LIAYQPLLSNTLAELYVREYMREQDRKPRNATPAPLREAP |
| | | | SANASVERIKTTSSIEFARLQFTYNHIQRHVNDMLGRIAV |
| | | | AWCELQNHELTLWNEARKLNPNAIASATVGRRVSARMLGD |
| | | | VMAVSTCVPVAPDNVIVQNSMRVSSRPGTCYSRPLVSFRY |
| | | | EDQGPLIEGQLGENNELRLTRDALEPCTVGHRRYFIFGGG |
| | | | YVYFEEYAYSHQLSRADVTTVSTFIDLNITMLEDHEFVPL |
| | | | EVYTRHEIKDSGLLDYTEVQRRNQLHDLRFADIDTVIRAD |
| | | | ANAAMFAGLCAFFEGMGDLGRAVGKVVMGVVGGVVSAVSG |
| | | | VSSFMSNPFGALAVGLLVLAGLVAAFFAFRYVLQLQRNPM |
| | | | KALYPLTTKELKTSDPGGVGGEGEEGAEGGGFDEAKLAEA |
| | | | REMIRYMALVSAMERTEHKARKKGTSALLSSKVTNMVLRK |
| | | | RNKARYSPLHNEDEAGDEDEL |
| 10 | UL29 | HG52 | MDTKPKTTTTVKVPPGPMGYVYGRACPAEGLELLSLLSAR |
| | | | SGDADVAVAPLIVGLTVESGFEANVAAVVGSRTTGLGGTA |
| | | | VSLKLMPSHYSPSVYVFHGGRHLAPSTQAPNLTRLCERAR |
| | | | PHFGFADYAPRPCDLKHETTGDALCERLGLDPDRALLYLV |
| | | | ITEGFREAVCISNTFLHLGGMDKVTIGDAEVHRIPVYPLQ |
| | | | MFMPDFSRVIADPFNCNHRSIGENFNYPLPFFNRPLARLL |
| | | | FEAVVGPAAVALRARNVDAVARAAAHLAFDENHEGAALPA |
| | | | DITFTAFEASQGKPQRGARDAGNKGPAGGFEQRLASVMAG |
| | | | DAALALESIVSMAVFDEPPPDITTWPLLEGQETPAARAGA |
| | | | VGAYLARAAGLVGAMVESTNSALHLTEVDDAGPADPKDHS |
| | | | KPSFYRFFLVPGTHVAANPQLDREGHVVPGYEGRPTAPLV |
| | | | GGTQEFAGEHLAMLCGFSPALLAKMLFYLERCDGGVIVGR |
| | | | QEMDVFRYVADSGQTDVPCNLCTFETRHACAHTTLMRLRA |
| | | | RHPKFASAARGAIGVFGTMNSAYSDCDVLGNYAAFSALKR |
| | | | ADGSENTRTIMQETYRAATERVMAELEALQYVDQAVPTAL |
| | | | GRLETIIGNREALHTVVNNIKQLVDREVEQLMRNLIEGRN |
| | | | FKFRDGLAEANHAMSLSLDPYTCGPCPLLQLLARRSNLAV |
| | | | YQDLALSQCHGVFAGQSVEGRNFRNQFQPVLRRRVMDLFN |
| | | | NGFLSAKTLTVALSEGAAICAPSLTAGQTAPAESSFEGDV |
| | | | ARVTLGFPKELRVKSRVLFAGASANASEAAKARVASLQSA |
| | | | YQKPDKRVDILLGPLGFLLKQFHAVIFPNGKPPGSNQPNP |
| | | | QWFWTALQRNQLPARLLSREDIETIAFIKRFSLDYGAINF |
| | | | INLAPNNVSELAMYYMANQILRYCDHSTYFINTLTAVIAG |
| | | | SRRPPSVQAAAAWAPQGGAGLEAGARALMDSLDAHPGAWT |
| | | | SMFASCNLLRPVMAARPMVVLGLSISKYYGMAGNDRVFQA |
| | | | GNWASLLGGKNACPLLIFDRTRKFVLACPRAGFVCAASSL |
| | | | GGGAHEHSLCEQLRGIIAEGGAAVASSVFVATVKSLGPRT |
| | | | QQLQIEDWLALLEDEYLSEEMMEFTTRALERGHGEWSTDA |
| | | | ALEVAHEAEALVSQLGAAGEVFNFGDFGDEDDHAASFGGL |
| | | | AAAAGAAGVARKRAFHGDDPFGEGPPEKKDLTLDML |
| 11 | UL29 | 333 | MDTKPKTTTTVKVPPGPMGYVYGRACPAEGLELLSLLSAR |
| | | | SGDADVAVAPLIVGLTVESGFEANVAAVVGSRTTGLGGTA |
| | | | VSLKLMPSHYSPSVYVFHGGRHLAPSTQAPNLTRLCERAR |
| | | | RHFGFSDYAPRPCDLKHETTGDALCERLGLDPDRALLYLV |
| | | | ITEGFREAVCISNTFLHLGGMDKVTIGDAEVHRIPVYPLQ |
| | | | MFMPDFSRVIADPFNCNHRSIGENFNYPLPFFNRPLARLL |
| | | | FEAVVGPAAVALRARNVDAVARAAAHLAFDENHEGAALPA |
| | | | DITFTAFEASQGKPQRGARDAGNKGPAGGFEQRLASVMAG |
| | | | DAALALESIVSMAVFDEPPPDITTWPLLEGQETPAARAGA |
| | | | VGAYLARAAGLVGAMVESTNSALHLTEVDDAGPADPKDHS |
| | | | KPSFYRFFLVPGTHVAANPQLDREGHVVPGYEGRPTAPLV |
| | | | GGTQEFAGEHLAMLCGFSPALLAKMLFYLERCDGGVIVGR |
| | | | QEMDVFRYVADSGQTDVPCNLCTFETRHACAHTTLMRLRA |
| | | | RHPKFASAARGAIGVFGTMNSAYSDCDVLGNYAAFSALKR |
| | | | ADGSENTRTIMQETYRAATERVMAELEALQYVDQAVPTAL |
| | | | GRLETIIGTREALHTVVNNIKQLVDREVEQLMRNLIEGRN |
| | | | FKFRDGLAEANHAMSLSLDPYTCGPCPLLQLLARRSNLAV |
| | | | YQDLALSQCHGVFAGQSVEGRNFRNQFQPVLRRRVMDLFN |
| | | | NGFLSAKTLTVALSEGAAICAPSLTAGQTAPAESSFEGDV |
| | | | ARVTLGFPKELRVKSRVLFAGASANASEAAKARVASLQSA |
| | | | YQKPDKRVDILLGPLGFLLKQFHAVIFPNGKPPGSNQPNP |
| | | | QWFWTALQRNQLPARLLSREDIETIAFIKRFSLDYGAINF |
| | | | INLAPNNVSELAMYYMANQILRYCDHSTYFINTLTAVIAG |
| | | | SRRPPGVQAAAAWAPQGGAGLEAGARALMDSLDAHPGAWT |
| | | | SMFASCNLLRPVMAARPMVVLGLSISKYYGMAGNDRVFQA |
| | | | GNWASLLGGKNACPLLIFDRTRKFVLACPRAGFVCAASSL |
| | | | GGGAHEHSLCEQLRGIIAEGGAAVASSVFVATVKSLGPRT |
| | | | QQLQIEDWLALLEDEYLSEEMMEFTTRALERGHGEWSTDA |
| | | | ALEVAHEAEALVSQLGAAGEVENFGDFGDEDDHAASFGGL |
| | | | AAAAGAAGVARKRAFHGDDPFGEGPPEKKDLTLDML |
| 12 | UL29 | MS | MDTKPKTTTTVKVPPGPMGYVYGRACPAEGLELLSLLSAR |
| | | | SGDADVAVAPLIVGLTVESGFEANVAAVVGSRTTGLGGTA |
| | | | VSLKLMPSHYSPSVYVFHGGRHLAPSTQAPNLTRLCERAR |
| | | | RHFGFSDYAPRPCDLKHETTGDALCERLGLDPDRALLYLV |
| | | | ITEGFREAVCISNTFLHLGGMDKVTIGDAEVHRIPVYPLQ |
| | | | MFMPDFSRVIADPFNCNHRSIGENFNYPLPFFNRPLARLL |
| | | | FEAVVGPAAVALRARNVDAVARAAAHLAFDENHEGAALPA |
| | | | DITFTAFEASQGKPQRGARDAGNKGPAGGFEQRLASVMAG |
| | | | DAALALESIVSMAVFDEPPPDITTWPLLEGQETPAARAGA |
| | | | VGAYLARAAGLVGAMVESTNSALHLTEVDDAGPADPKDHS |
| | | | KPSFYRFFLVPGTHVAANPQLDREGHVVPGYEGRPTAPLV |
| | | | GGTQEFAGEHLAMLCGFSPALLAKMLFYLERCDGGVIVGR |
| | | | QEMDVFRYVADSGQTDVPCSLCTFETRHACAHTTLMRLRA |
| | | | RHPKFASAARGAIGVFGTMNSAYSDCDVLGNYAAFSALKR |
| | | | ADGSENTRTIMQETYRAATERVMAELEALQYVDQAVPTAL |
| | | | GRLETIIGNREALHTVVNNIKQLVDREVEQLMRNLIEGRN |
| | | | FKFRDGLAEANHAMSLSLDPYTCGPCPLLQLLARRSNLAV |
| | | | YQDLALSQCHGVFAGQSVEGRNFRNQFQPVLRRRVMDLFN |
| | | | NGFLSAKTLTVALSEGAAICAPSLTAGQTAPAESSFEGDV |
| | | | ARVTLGFPKELRVKSRVLFAGASANASEAAKARVASLQSA |
| | | | YQKPDKRVDILLGPLGFLLKQFHAVIFPNGKPPGSNQPNP |
| | | | QWFWTALQRNQLPARLLSREDIETIAFIKRFSLDYGAINF |
| | | | INLAPNNVSELAMYYMANQILRYCDHSTYFINTLTAVIAG |
| | | | SRRPPSVQAAAAWAPQGGAGLEAGARALMDSLDAHPGAWT |
| | | | SMFASCNLLRPVMAARPMVVLGLSISKYYGMAGNDRVFQA |
| | | | GNWASLLGGKNACPLLIFDRTRKFVLACPRAGFVCAASSL |
| | | | GGGAHEHSLCEQLRGIIAEGGAAVASSVFVATVKSLGPRT |
| | | | QQLQIEDWLALLEDEYLSEEMMEFTTRALERGHGEWSTDA |
| | | | ALEVAHEAEALVSQLGAAGEVENFGDFGDEDDHAASFGGL |
| | | | AAAGAGAAGVARKRAFHGDDPFGEGPPEKKDLTLDML |
| 13 | UL39 | HG52 | MANRPAASALAGARSPSERQEPREPEVAPPGGDHVFCRKV |
| | | | SGVMVLSSDPPGPAAYRISDSSFVQCGSNCSMIIDGDVAR |
| | | | GHLRDLEGATSTGAFVAISNVAAGGDGRTAVVALGGTSGP |
| | | | SATTSVGTQTSGEFLHGNPRTPEPQGPQAVPPPPPPPFPW |
| | | | GHECCARRDARGGAEKDVGAAESWSDGPSSDSETEDSDSS |
| | | | DEDTGSETLSRSSSIWAAGATDDDDSDSDSRSDDSVQPDV |
| | | | VVRRRWSDGPAPVAFPKPRRPGDSPGNPGLGAGTGPGSAT |
| | | | DPRASADSDSAAHAAAPQADVAPVLDSQPTVGTDPGYPVP |
| | | | LELTPENAEAVARFLGDAVDREPALMLEYFCRCAREESKR |
| | | | VPPRTFGSAPRLTEDDFGLLNYALAEMRRLCLDLPPVPPN |
| | | | AYTPYHLREYATRLVNGFKPLVRRSARLYRILGVLVHLRI |
| | | | RTREASFEEWMRSKEVDLDFGLTERLREHEAQLMILAQAL |
| | | | NPYDCLIHSTPNTLVERGLQSALKYEEFYLKRFGGHYMES |
| | | | VFQMYTRIAGFLACRATRGMRHIALGRQGSWWEMFKFFFH |
| | | | RLYDHQIVPSTPAMLNLGTRNYYTSSCYLVNPQATTNQAT |
| | | | LRAITGNVSAILARNGGIGLCMQAFNDASPGTASIMPALK |
| | | | VLDSLVAAHNKQSTRPTGACVYLEPWHSDVRAVLRMKGVL |
| | | | AGEEAQRCDNIFSALWMPDLFFKRLIRHLDGEKNVTWSLF |
| | | | DRDTSMSLADFHGEEFEKLYEHLEAMGFGETIPIQDLAYA |
| | | | IVRSAATTGSPFIMFKDAVNRHYIYDTQGAAIAGSNLCTE |
| | | | IVHPASKRSSGVCNLGSVNLARCVSRQTFDFGRLRDAVQA |
| | | | CVLMVNIMIDSTLQPTPQCTRGNDNLRSMGIGMQGLHTAC |
| | | | LKMGLDLESAEFRDLNTHIAEVMLLAAMKTSNALCVRGAR |
| | | | PFSHFKRSMYRAGRFHWERFSNASPRYEGEWEMLRQSMMK |
| | | | HGLRNSQFIALMPTAASAQISDVSEGFAPLFTNLFSKVTR |
| | | | DGETLRPNTLLLKELERTFGGKRLLDAMDGLEAKQWSVAQ |
| | | | ALPCLDPAHPLRRFKTAFDYDQELLIDLCADRAPYVDHSQ |
| | | | SMTLYVTEKADGTLPASTLVRLLVHAYKRGLKTGMYYCKV |
| | | | RKATNSGVFAGDDNIVCTSCAL |
| 14 | UL39 | 333 | MANRPAASALAGARSPSERQEPREPEVAPPGGDHVFCRKV |
| | | | SGVMVLSSDPPGPAAYRISDSSFVQCGSNCSMIIDGDVAR |
| | | | GHLRDLEGATSTGAFVAISNVAAGGDGRTAVVALGGTSGP |
| | | | SATTSVGTQTSGEFLHGNPRTPEPQGPQAVPPPPPPPFPW |
| | | | GHECCARRDARGGAEKDVGAAESWSDGPSSDSETEDSDSS |
| | | | DEDTGSGSETLSRSSSIWAAGATDDDDSDSDSRSDDSVQP |
| | | | DVVVRRRWSDGPAPVAFPKPRRPGDSPGNPGLGAGTGPGS |
| | | | ATDPRASADSDSAAHAAAPQADVAPVLDSQPTVGTDPGYP |
| | | | VPLELTPENAEAVARFLGDAVDREPALMLEYFCRCAREES |
| | | | KRVPPRTFGSAPRLTEDDFGLLNYALAEMRRLCLDLPPVP |
| | | | PNAYTPYHLREYATRLVNGFKPLVRRSARLYRILGILVHL |
| | | | RIRTREASFEEWMRSKEVDLDFGLTERLREHEAQLMILAQ |
| | | | ALNPYDCLIHSTPNTLVERGLQSALKYEEFYLKRFGGHYM |
| | | | ESVFQMYTRIAGFLACRATRGMRHIALGRQGSWWEMFKFF |
| | | | FHRLYDHQIVPSTPAMLNLGTRNYYTSSCYLVNPQATTNQ |
| | | | ATLRAITGNVSAILARNGGIGLCMQAFNDASPGTASIMPA |
| | | | LKVLDSLVAAHNKQSTRPTGACVYLEPWHSDVRAVLRMKG |
| | | | VLAGEEAQRCDNIFSALWMPDLFFKRLIRHLDGEKNVTWS |
| | | | LFDRDTSMSLADFHGEEFEKLYEHLEAMGFGETIPIQDLA |
| | | | YAIVRSAATTGSPFIMFKDAVNRHYIYDTQGAAIAGSNLC |
| | | | TEIVHPSSKRSSGVCNLGSVNLARCVSRRTFDFGMLRDAV |
| | | | QACVLMVNIMIDSTLQPTPQCARGHDNLRSMGIGMQGLHT |
| | | | ACLKMGLDLESAEFRDLNTHIAEVMLLAAMKTSNALCVRG |
| | | | ARPFSHFKRSMYRAGRFHWERFSNASPRYEGEWEMLRQSM |
| | | | MKHGLRNSQFIALMPTAASAQISDVSEGFAPLFTNLFSKV |
| | | | TRDGETLRPNTLLLKELERTFGGKRLLDAMDGLEAKQWSV |
| | | | AQALPCLDPAHPLRRFKTAFDYDQELLIDLCADRAPYVDH |
| | | | SQSMTLYVTEKADGTLPASTLVRLLVHAYKRGLKTGMYYC |
| | | | KVRKATNSGVFAGDDNIVCTSCAL |
| 15 | UL39 | MS | MANRPAASALAGARSPSERQEPREPEVAPPGGDHVFCRKV |
| | | | SGVMVLSSDPPGPAAYRISDSSFVQCGSNCSMIIDGDVAR |
| | | | GHLRDLEGATSTGAFVAISNVAAGGDGRTAVVALGGTSGP |
| | | | SATTSVGTQTSGEFLHGNPRTPEPQGPQAVPPPPPPPFPW |
| | | | GHECCARRDARGGAEKDVGAAESWSDGPSSDSETEDSDSS |
| | | | DEDTGSGSETLSRSSSIWAAGATDDDDSDSDSRSDDSVQP |
| | | | DVVVRRRWSDGPAPVAFPKPRRPGDSPGNPGLGAGTGPGS |
| | | | ATDPRASADSDSAAHAAAPQADVAPVLDSQPTVGTDPGYP |
| | | | VPLELTPENAEAVARFLGDAVDREPALMLEYFCRCAREES |
| | | | KRVPPRTFGSAPRLTEDDFGLLNYALAEMRRLCLDLPPVP |
| | | | PNAYTPYHLREYATRLVNGFKPLVRRSARLYRILGVLVHL |
| | | | RIRTREASFEEWMRSKEVDLDFGLTERLREHEAQLMILAQ |
| | | | ALNPYDCLIHSTPNTLVERGLQSALKYEEFYLKRFGGHYM |
| | | | ESVFQMYTRIAGFLACRATRGMRHIALGRQGSWWEMFKFF |
| | | | FHRLYDHQIVPSTPAMLNLGTRNYYTSSCYLVNPQATTNQ |
| | | | ATLRAITGNVSAILARNGGIGLCMQAFNDASPGTASIMPA |
| | | | LKVLDSLVAAHNKQSTRPTGACVYLEPWHSDVRAVLRMKG |
| | | | VLAGEEAQRCDNIFSALWMPDLFFKRLIRHLDGEKNVTWS |
| | | | LFDRDTSMSLADFHGEEFEKLYEHLEAMGFGETIPIQDLA |
| | | | YAIVRSAATTGSPFIMFKDAVNRHYIYDTQGAAIAGSNLC |
| | | | TEIVHPSCKRSSGVCNLGSVNLARCVSRRTFDFGMLRDAV |
| | | | QACVLMVNIMIDSTLQPTPQCARGHDNLRSMGIGMQGLHT |
| | | | ACLKMGLDLESAEFRDLNTHIAEVMLLAAMKTSNALCVRG |
| | | | ARPFSHFKRSMYRAGRFHWERFSNASPRYEGEWEMLRQSM |
| | | | MKHGLRNSQFIALMPTAASAQISDVSEGFAPLFTNLFSKV |
| | | | TRDGETLRPNTLLLKELERTFGGKRLLDAMDGLEAKQWSV |
| | | | AQALPCLDPAHPLRRFKTAFDYDQELLIDLCADRAPYVDH |
| | | | SQSMTLYVTEKADGTLPASTLVRLLVHAYKRGLKTGMYYC |
| | | | KVRKATNSGVFAGDDNIVCTSCAL |
| 16 | UL40 | HG52 | MDPAVSPASTDPLDTHASGAGAAPIPVCPTPERYFYTSQC |
| | | | PDINHLRSLSILNRWLETELVFVGDEEDVSKLSEGELGFY |
| | | | RFLFAFLSAADDLVTENLGGLSGLFEQKDILHYYVEQECI |
| | | | EVVHSRVYNIIQLVLFHNNDQARRAYVARTINHPAIRVKV |
| | | | DWLEARVRECDSIPEKFILMILIEGVFFAASFAAIAYLRT |
| | | | NNLLRVTCQSNDLISRDEAVHTTASCYIYNNYLGGHAKPE |
| | | | AARVYRLFREAVDIEIGFIRSQAPTDSSILSPGALAAIEN |
| | | | YVRESADRLLGLIHMQPLYSAPAPDASFPLSLMSTDKHTN |
| | | | FFECRSTSYAGAVVNDL |
| 17 | UL40 | 333 | MDPAVSPASTDPLDTHASGAGAAPIPVCPTPERYFYTSQC |
| | | | PDINHLRSLSILNRWLETELVFVGDEEDVSKLSEGELGFY |
| | | | RFLFAFLSAADDLVTENLGGLSGLFEQKDILHYYVEQECI |
| | | | EVVHSRVYNIIQLVLFHNNDQARRAYVARTINHPAIRVKV |
| | | | DWLEARVRECDSIPEKFILMILIEGVFFAASFAAIAYLRT |
| | | | NNLLRVTCQSNDLISRDEAVHTTASCYIYNNYLGGHAKPE |
| | | | AARVYRLFREAVDIEIGFIRSQAPTDSSILSPGALAAIEN |
| | | | YVRESADRLLGLIHMQPLYSAPAPDASFPLSLMSTDKHTN |
| | | | FFECRSTSYAGAVVNDL |
| 18 | UL40 | MS | MDPAVSPASTDPLDTHASGAGAAPIPVCPTPERYFYTSQC |
| | | | PDINHLRSLSILNRWLETELVFVGDEEDVSKLSEGELGFY |
| | | | RFLFAFLSAADDLVTENLGGLSGLFEQKDILHYYVEQECI |
| | | | EVVHSRVYNIIQLVLFHNNDQARRAYVARTINHPAIRVKV |
| | | | DWLEARVRECASIPEKFILMILIEGVFFAASFAAIAYLRI |
| | | | NNLLRVTCQSNDLISRDEAVHTTASCYIYNNYLGGHAKPE |
| | | | AARVYRLFREAVDIEIGFIRSQAPTDSSILSPGALAAIEN |
| | | | YVRFSADRLLGLIHMQPLYSAPAPDASFPLSLMSTDKHTN |
| | | | FFECRSTSYAGAVVNDL |
| 19 | UL46 | HG52 | MQRRARGASSLRLARCLTPANLIRGANAGVPERRIFAGCL |
| | | | LPTPEGLLSAAVGVLRQRADDLQPAFLTGADRSVRLAARH |
| | | | HNTVPESLIVDGLASDPHYDYIRHYASAAKQALGEVELSG |
| | | | GQLSRAILAQYWKYLQTVVPSGLDIPDDPAGDCDPSLHVL |
| | | | LRPTLLPKLLVRAPFKSGAAAAKYAAAVAGLRDAAHRLQQ |
| | | | YMFFMRPADPSRPSTDTALRLSELLAYVSVLYHWASWMLW |
| | | | TADKYVCRRLGPADRRFVALSGSLEAPAETFARHLDRGPS |
| | | | GTTGSMQCMALRAAVSDVLGHLTRLAHLWETGKRSGGTYG |
| | | | IVDAIVSTVEVLSIVHHHAQYIINATLTGYVVWASDSLNN |
| | | | EYLTAAVDSQERFCRTAAPLFPTMTAPSWARMELSIKSWF |
| | | | GAALAPDLLRSGTPSPHYESILRLAASGPPGGRGAVGGSC |
| | | | RDKIQRTRRDNAPPPLPRARPHSTPAAPRRCRRHREDLPE |
| | | | PPHVDAADRGPEPCAGRPATYYTHMAGAPPRLPPRNPAPP |
| | | | EQRPAAAARPLAAQREAAGVYDAVRTWGPDAEAEPDQMEN |
| | | | TYLLPDDDAAMPAGVGLGATPAADTTAAAAWPAESHAPRA |
| | | | PSEDADSIYESVGEDGGRVYEEIPWVRVYENICPRRRLAG |
| | | | GAALPGDAPDSPYIEAENPLYDWGGSALFSPRRATRAPDP |
| | | | GLSLSPMPARPRTNALANDGPTNVAALSALLTKLKRGRHQ |
| | | | SH |
| 20 | UL46 | 333 | MQRRARGASSLRLARCLTPANLIRGANAGVPERRIFAGCL |
| | | | LPTPEGLLSAAVGVLRQRADDLQPAFLTGADRSVRLAARH |
| | | | HNTVPESLIVDGLASDPHYDYIRHYASAAKQALGEVELSG |
| | | | GQLSRAILAQYWKYLQTVVPSGLDIPDDPAGDCDPSLHVL |
| | | | LRPTLLPKLLVRAPFKSGAAAAKYAAAVAGLRDAAHRLQQ |
| | | | YMFFMRPADPSRPSTDTALRLSELLAYVSVLYHWASWMLW |
| | | | TADKYVCRRLGPADRRFVALSGSLEAPAETFARHLDRGPS |
| | | | GTTGSMQCMALRAAVSDVLGHLTRLAHLWETGKRSGGTYG |
| | | | IVDAIVSTVEVLSIVHHHAQYIINATLTGYVVWASDSLNN |
| | | | EYLRAAVDSQERFCRTAAPLFPTMTAPSWARMELSIKSWF |
| | | | GAALAPDLLRSGTPSPHYESILRLAASGPPGGRGAVGGSC |
| | | | RDKIQRTRRDNAPPPLPRARPHSTPAAPRRFRRHREDLPE |
| | | | PPHVDAADRGPEPCAGRPATYYTHMAGAPPRLPPRNPAPP |
| | | | EQRPAAAARPLAAQREAAGVYDAVRTWGPDAEAEPDQMEN |
| | | | TYLLPDDDAAMPAGVGLGATPAADTTAAAWPAESHAPRAP |
| | | | SEDADSIYESVSEDGGRVYEEIPWVRVYENICLRRQDAGG |
| | | | AAPPGDAPDSPYIEAENPLYDWGGSALFSPPGATRAPDPG |
| | | | LSLSPMPARPRTNALANDGPTNVAALSALLTKLKRGRHQS |
| | | | H |
| 21 | UL46 | MS | MQRRARGASSLRLARCLTPANLIRGANAGVPERRIFAGCL |
| | | | LPTPEGLLSAAVGVLRQRADDLQPAFLTGADRSVRLAARH |
| | | | HNTVPESLIVDGLASDPHYDYIRHYASAAKQALGEVELSG |
| | | | GQLSRAILAQYWKYLQTVVPSGLDIPDDPAGDCDPSLHVL |
| | | | LRPTLLPKLLVRAPFKSGAAAAKYAAAVAGLRDATHRLQQ |
| | | | YMFFMRPADPSRPSTDTALRLSELLAYVSVLYHWASWMLW |
| | | | TADKYVCRRLGPADRRFVALSGSLEAPAETFARHLDRGPS |
| | | | GTTGSMQCMALRAAVSDVLGHLTRLAHLWETGKRSGGTYG |
| | | | IVDAIVSTVEVLSIVHHHAQYIINATLTGYVVWASDSLNN |
| | | | EYLRAAVDSQERFCRTAAPLFPTMTAPSWARMELSIKSWF |
| | | | GAALAPDLLRSGTPSPHYESILRLAASGPPGGRGAVGGSC |
| | | | RDKIQRTRRDNAPPPLPRARPHSTPAAPRRFRRHREDLPE |
| | | | PPHVDAADRGPEPCAGRPATYYTHMAGAPPRLPPRNPAPP |
| | | | EQRPAAAARPLAAQREAAGVYDAVRTWGPDAEAEPDQMEN |
| | | | TYLLPDDDAAMPAGVGLGATPAADTTAAAWPAKGHAPRAP |
| | | | SEDADSIYESVGEDGGRVYEEIPWVRVYENICLRRQDAGG |
| | | | AAPPGDAPDSPYIEAENPLYDWGGSALFSPPGATRAPDPG |
| | | | LSLSPMPARPRTNALANDGPTNVAALSALLTKLKRGRHQS |
| | | | H |
| 22 | UL47 | HG52 | MSVRGHAVRRRRASTRSHAPSAHRADSPVEDEPEGGGVGL |
| | | | MGYLRAVFNVDDDSEVEAAGEMASEEPPPRRRREARGHPG |
| | | | SRRASEARAAAPPRRASFPRPRSVTARSQSVRGRRDSAIT |
| | | | RAPRGGYLGPMDPRDVLGRVGGSRVVPSPLFLDELNYEED |
| | | | DYPAAVAHDDGPGARPSATVEILAGRVSGPELQAAFPLDR |
| | | | LTPRVAAWDESVRSALALGHPAGFYPCPDSAFGLSRVGVM |
| | | | HFASPADPKVFFRQTLQQGEALAWYVTGDAILDLTDRRAK |
| | | | TSPSRAMGFLVDAIVRVAINGWVCGTRLHTEGRGSELDDR |
| | | | AAELRRQFASLTALRPVGAAAVPLLSAGGAAPPHPGPDAA |
| | | | VFRSSLGSLLYWPGVRALLGRDCRVAARYAGRMTYIATGA |
| | | | LLARFNPGAVKCVLPREAAFAGRVLDVLAVLAEQTVQWLS |
| | | | VVVGARLHPHSAHPAFADVEQEALFRALPLGSPGVVAAEH |
| | | | EALGDTAARRLLATSGLNAVLGAAVYALHTALATVTLKYA |
| | | | LACGDARRRRDDAAAARAVLATGLILQRLLGLADTVVACV |
| | | | ALAAFDGGSTAPEVGTYTPLRYACVLRATQPLYARTTPAK |
| | | | FWADVRAAAEHVDLRPASSAPRAPVSGTADPAFLLEDLAA |
| | | | FPPAPLNSESVLGPRVRVVDIMAQFRKLLMGDEETAALRA |
| | | | HVSGRRATGLGGPPRP |
| 23 | UL47 | 333 | MSVRGHAVRRRRASTRSHAPSAHRADSPVEDEPEGGGVGL |
| | | | MGYLRAVFNVDDDSEVEAAGEMASEEPPPRRRREARGHPG |
| | | | SRRASEARAAAPPRRASFPRPRSVTARSQSVRGRRDSAIT |
| | | | RAPRGGYLGPMDPRDVLGRVGGSRVVPSPLFLDELSYEED |
| | | | DYPAAVAHDDGAGARPPATVEILAGRVSGPELQAAFPLDR |
| | | | LTPRVAAWDESVRSALALGHPAGFYPCPDSAFGLSRVGVM |
| | | | HFASPADPKVFFRQTLQQGEALAWYVTGDAILDLTDRRAK |
| | | | TSPSRAMGFLVDAIVRVAINGWVCGTRLHTEGRGSELDDR |
| | | | AAELRRQFASLTALRPVGAAAVPLLSAGGAAPPHPGPDAA |
| | | | VFRSSLGSLLYWPGVRALLGRDCRVAARYAGRMTYIATGA |
| | | | LLARENPGAVKCVLPREAAFAGRVLDVLAVLAEQTVQWLS |
| | | | VVVGARLHPHSAHPAFADVEQEALFRALPLGSPGVVAAEH |
| | | | EALGDTAARRLLATSGLNAVLGAAVYALHTALATVTLKYA |
| | | | LACGDARRRRDDAAAARAVLATGLILQRLLGLADTVVACV |
| | | | ALAAFDGGSTAPEVGTYTPLRYACVLRATQPLYARTTPAK |
| | | | FWADVRAAAEHVDLRPASSAPRAPVSGTADPAFLLEDLAA |
| | | | FPPAPLNSESVLGPRVRVVDIMAQFRKLLMGDEETAALRA |
| | | | HVSGRRATGLGGPPRP |
| 24 | UL47 | MS | MSVRGHAVRRRRASTRSHAPSAHRADSPVEDEPEGGGVGL |
| | | | MGYLRAVFNVDDDSEVEAAGEMASEEPPPRRRREARGHPG |
| | | | SRRASEARAAAPPRRASFPRPRSVTARSQSVRGRRDSAIT |
| | | | RAPRGGYLGPMDPRDVLGRVGGSRVVPSPLFLDELSYEED |
| | | | DYPAAVAHDDGAGARPPATVEILAGRVSGPELQAAFPLDR |
| | | | LTPRVAAWDESVRSALALGHPAGFYPCPDSAFGLSRVGVM |
| | | | HFASPADPKVFFRQTLQQGEALAWYVTGDAILDLTDRRAK |
| | | | TSPSRAMGFLVDAIVRVAINGWVCGTRLHTEGRGSELDDR |
| | | | AAELRRQFASLTALRPVGAAAVPLLSAGGAAPPHPGPDAA |
| | | | VFRSSLGSLLYWPGVRALLGRDCRVAARYAGRMTYIATGA |
| | | | LLARFNPGAVKCVLPREAAFAGRVLDVLAVLAEQTVQWLS |
| | | | VVVGARLHPHSAHPAFADVEQEALFRALPLGSPGVVAAEH |
| | | | EALGDTAARRLLATSGLNAVLGAAVYALHTALATVTLKYA |
| | | | LACGDARRRRDDAAAARAVLATGLILQRLLGLADTVVACV |
| | | | ALAAFDGGSTAPEVGTYTPLRYACVLRATQPLYARTTPAK |
| | | | FWADVRAAAEHVDLRPASSAPRAPVSGTADPAFLLEDLAA |
| | | | FPPAPLNSESVLGPRVRVVDIMAQFRKLLMGDEETAALRA |
| | | | HVSGRRATGLGGPPRP |
| 25 | UL48 | HG52 | MDLLVDDLFADADGVSPPPPRPAGGPKNTPAAPPLYATGR |
| | | | LSQAQLMPSPPMPVPPAALFNRLLDDLGFSAGPALCTMLD |
| | | | TWNEDLFSGFPTNADMYRECKFLSTLPSDVIDWGDAHVPE |
| | | | RSPIDIRAHGDVAFPTLPATRDELPSYYEAMAQFFRGELR |
| | | | AREESYRTVLANFCSALYRYLRASVRQLHRQAHMRGRNRD |
| | | | LREMLRTTIADRYYRETARLARVLFLHLYLFLSREILWAA |
| | | | YAEQMMRPDLFDGLCCDLESWRQLACLFQPLMFINGSLTV |
| | | | RGVPVEARRLRELNHIREHLNLPLVRSAAAEEPGAPLTTP |
| | | | PVLQGNQARSSGYFMLLIRAKLDSYSSVATSEGESVMREH |
| | | | AYSRGRTRNNYGSTIEGLLDLPDDDDAPAEAGLVAPRMSF |
| | | | LSAGQRPRRLSTTAPITDVSLGDELRLDGEEVDMTPADAL |
| | | | DDFDLEMLGDVESPSPGMTHDPVSYGALDVDDFEFEQMFT |
| | | | DAMGIDDFGG |
| 26 | UL48 | 333 | MDLLVDDLFADADGVSPPPPRPAGGPKNTPAAPPLYATGR |
| | | | LSQAQLMPSPPMPVPPAALFNRLLDDLGFSAGPALCTMLD |
| | | | TWNEDLFSGFPTNADMYRECKFLSTLPSDVIDWGDAHVPE |
| | | | RSPIDIRAHGDVAFPTLPATRDELPSYYEAMAQFFRGELR |
| | | | AREESYRTVLANFCSALYRYLRASVRQLHRQAHMRGRNRD |
| | | | LREMLRTTIADRYYRETARLARVLFLHLYLFLSREILWAA |
| | | | YAEQMMRPDLFDGLCCDLESWRQLACLFQPLMFINGSLTV |
| | | | RGVPVEARRLRELNHIREHLNLPLVRSAAAEEPGAPLTTP |
| | | | PVLQGNQARSSGYFMLLIRAKLDSYSSVATSEGESVMREH |
| | | | AYSRGRTRNNYGSTIEGLLDLPDDDDAPAEAGLVAPRMSF |
| | | | LSAGQRPRRLSTTAPITDVSLGDELRLDGEEVDMTPADAL |
| | | | DDFDLEMLGDVESPSPGMTHDPVSYGALDVDDFEFEQMFT |
| | | | DAMGIDDFGG |
| 27 | UL48 | MS | MDLLVDDLFADADGVSPPPPRPAGGPKNTPAAPPLYATGR |
| | | | LSQAQLMPSPPMPVPPAALFNRLLDDLGFSAGPALCTMLD |
| | | | TWNEDLFSGFPTNADMYRECKFLSTLPSDVIDWGDAHVPE |
| | | | RSPIDIRAHGDVAFPTLPATRDELPSYYEAMAQFFRGELR |
| | | | AREESYRTVLANFCSALYRYLRASVRQLHRQAHMRGRNRD |
| | | | LREMLRTTIADRYYRETARLARVLFLHLYLFLSREILWAA |
| | | | YAEQMMRPDLFDGLCCDLESWRQLACLFQPLMFINGSLTV |
| | | | RGVPVEARRLRELNHIREHLNLPLVRSAAAEEPGAPLTTP |
| | | | PVLQGNQARSSGYFMLLIRAKLDSYSSVATSEGESVMREH |
| | | | AYSRGRTRNNYGSTIEGLLDLPDDDDAPAEAGLVAPRMSF |
| | | | LSAGQRPRRLSTTAPITDVSLGDELRLDGEEVDMTPADAL |
| | | | DDFDLEMLGDVESPSPGMTHDPVSYGALDVDDFEFEQMFT |
| | | | DAMGIDDFGG |
| 28 | UL49 | HG52 | MTSRRSVKSCPREAPRGTHEELYYGPVSPADPESPRDDFR |
| | | | RGAGPMRARPRGEVRFLHYDEAGYALYRDSSSDDDESRDT |
| | | | ARPRRSASVAGSHGPGPARAPPPPGGPVGAGGRSHAPPAR |
| | | | TPKMTRGAPKASATPATDPARGRRPAQADSAVLLDAPAPT |
| | | | ASGRTKTPAQGLAKKLHFSTAPPSPTAPWTPRVAGFNKRV |
| | | | FCAAVGRLAATHARLAAVQLWDMSRPHTDEDLNELLDLTT |
| | | | IRVTVCEGKNLLQRANELVNPDAAQDVDATAAARGRPAGR |
| | | | AAATARAPARSASRPRRPLE |
| 29 | UL49 | 333 | MTSRRSVKSCPREAPRGTHEELYYGPVSPADPESPRDDFR |
| | | | RGAGPMRARPRGEVRFLHYDEAGYALYRDSSSSEDNDESR |
| | | | DTARPRRSASVAGSHGPGPARAPPPPGGPVGAGGRSHAPP |
| | | | ARTPKMTRGAPKAPATPATDPARGRRPAQADSAVLLDAPA |
| | | | PTASGRTKTPAQGLAKKLHFSTAPPSPTAPWTPRVAGENK |
| | | | RVFCAAVGRLAATHARLAAVQLWDMSRPHTDEDLNELLDL |
| | | | TTIRVTVCEGKNLLQRANELVNPDAAQDVDATAAARGRPA |
| | | | GRAAATARAPARSASRPRRPLE |
| 30 | UL49 | MS | MTSRRSVKSCPREAPRGTHEELYYGPVSPADPESPRDDFR |
| | | | RGAGPMRARPRGEVRFLHYDEAGYALYRDSSSSEDDDESR |
| | | | DTARPRRSASVAGSHGPGPARAPPPPGGPVGAGGRSHAPP |
| | | | ARTPKMTRGAPKAPATPATDPARGRRPAQADSAVLLDAPA |
| | | | PTASGRTKTPAQGLAKKLHFSTAPPSPTAPWTPRVAGFNK |
| | | | RVFCAAVGRLAATHARLAAVQLWDMSRPHTDEDLNELLDL |
| | | | TTIRVTVCEGKNLLQRANELVNPDAAQDVDATAAARGRPA |
| | | | GRAAATARAPARSASRPRRPLE |
| 31 | RS1 | HG52 | MSAEQRKKKKTTTTTQGRGAEVAMADEDGGRLRAAAETTG |
| | [ICP4] | | GPGSPDPADGPPPTPNPDRRPAARPGFGWHGGPEENEDEA |
| | | | DDAAADADADEAAPASGEAVDEPAADGVVSPRQLALLASM |
| | | | VDEAVRTIPSPPPERDGAQEEAARSPSPPRTPSMRADYGE |
| | | | ENDDDDDDDDDDDRDAGRWVRGPETTSAVRGAYPDPMASL |
| | | | SPRPPAPRRHHHHHHHRRRRAPRRRSAASDSSKSGSSSSA |
| | | | SSASSSASSSSSASASSSDDDDDDDAARAPASAADHAAGG |
| | | | TLGADDEEAGVPARAPGAAPRPSPPRAEPAPARTPAATAG |
| | | | RLERRRARAAVAGRDATGRFTAGRPRRVELDADAASGAFY |
| | | | ARYRDGYVSGEPWPGAGPPPPGRVLYGGLGDSRPGLWGAP |
| | | | EAEEARARFEASGAPAPVWAPELGDAAQQYALITRLLYTP |
| | | | DAEAMGWLQNPRVAPGDVALDQACFRISGAARNSSSFISG |
| | | | SVARAVPHLGYAMAAGRFGWGLAHVAAAVAMSRRYDRAQK |
| | | | GFLLTSLRRAYAPLLARENAALTGARTPDDGGDANRHDGD |
| | | | DARGKPAAAAAPLPSAAASPADERAVPAGYGAAGVLAALG |
| | | | RLSAAPASAPAGADDDDDDDGAGGGGGGRRAEAGRVAVEC |
| | | | LAACRGILEALAEGFDGDLAAVPGLAGARPAAPPRPGPAG |
| | | | AAAPPHADAPRLRAWLRELRFVRDALVLMRLRGDLRVAGG |
| | | | SEAAVAAVRAVSLVAGALGPALPRSPRLLSSAAAAAADLL |
| | | | FQNQSLRPLLADTVAAADSLAAPASAPREARKRKSPAPAR |
| | | | APPGGAPRPPKKSRADAPRPAAAPPAGAAPPAPPTPPPRP |
| | | | PRPAALTRRPAEGPDPQGGWRRQPPGPSHTPAPSAAALEA |
| | | | YCAPRAVAELTDHPLFPAPWRPALMFDPRALASLAARCAA |
| | | | PPPGGAPAAFGPLRASGPLRRAAAWMRQVPDPEDVRVVIL |
| | | | YSPLPGEDLAAGRAGGGPPPEWSAERGGLSCLLAALGNRL |
| | | | CGPATAAWAGNWTGAPDVSALGAQGVLLLSTRDLAFAGAV |
| | | | EFLGLLAGACDRRLIVVNAVRAADWPADGPVVSRQHAYLA |
| | | | CEVLPAVQCAVRWPAARDLRRTVLASGRVFGPGVFARVEA |
| | | | AHARLYPDAPPLRLCRGANVRYRVRTRFGPDTLVPMSPRE |
| | | | YRRAVLPALDGRAAASGAGDAMAPGAPDFCEDEAHSHRAC |
| | | | ARWGLGAPLRPVYVALGRDAVRGGPAELRGPRREFCARAL |
| | | | LEPDGDAPPLVLRDDADAGPPPQIRWASAAGRAGTVLAAA |
| | | | GGGVEVVGTAAGLATPPRREPVDMDAELEDDDDGLFGE |
| 32 | RS1 | 333 | MSAEQRKKKKTTTTTQGRGAEVAMADEDGGRLRAAAETTG |
| | [ICP4] | | GPGSPDPADGPPPTPNPDRRPAARPGFGWHGGPEENEDED |
| | | | DDAAADADADEAAPASGEAVDEPAADGVVSPRQLALLASM |
| | | | VDEAVRTIPSPPPERDGAEEEAARSPSPPRTPSMCADYGE |
| | | | ENDDDDDDDDRDAGRWVRGPETTSAVRGAYPDPMASLSPR |
| | | | PPAPRRHHHHHHRRRRRRAPHRRSTASDSSKSGSSSSASS |
| | | | ASSSASSSSSASASSSDDDDDDAARAPASAADHAAGGTLG |
| | | | ADDEEAGVPARAPGAAPRPSPPRAEPAPGAGAGSARTPAA |
| | | | TAGRLERRRARAAVAGRDATGRFTAGRPRRVELDADAASG |
| | | | AFYARYRDGYVSGEPWPGAGPPPPGRVLYGGLGDSRPGLW |
| | | | GAPEAEEARARFEASGAPAPVWAPELGDAAQQYALITRLL |
| | | | YTPDAEAMGWLQNPRVAPGDVALDQACFRISGAARNSSSF |
| | | | ISGSVARAVPHLGYAMAAGRFGWGLAHVAAAVAMSRRYDR |
| | | | AQKGFLLTSLRRAYAPLLARENAALTGARTPDDGGDANRR |
| | | | DGDDARGKPAAAAAPLPSAAASPADERAVPAGYGAAGVLA |
| | | | ALGRLSAAPASAPAGADDDDDDDDGAGGGGGGRRAEAGRV |
| | | | AVECLAACRGILEALAEGFDGDLAAVPGLAGARPAAPPRP |
| | | | GPAGAAAPPHADAPRLRAWLRELRFVRDALVLMRLRGDLR |
| | | | VAGGSEAAVAAVRAVSLVAGALGPALPRSPRLLSSAAAAA |
| | | | ADLLFQNQSLRPLLADTVAAADSLAAPASAPREARKRKSP |
| | | | APARAPPGGAPRPPKKSRADAPRPAAAPPAGAAPPAPPTP |
| | | | PPRPPRPAALTRRPAEGPDPQGGWRRQPPGPSHTPAPSAA |
| | | | ALEAYCAPRAVAELTDHPLFPAPWRPALMFDPRALASLAA |
| | | | RCAAPPPGGAPAAFGPLRASGPLRRAAAWMRQVPDPEDVR |
| | | | VVILYSPLPGEDLAAGRAGGGPPPEWSAERGGLSCLLAAL |
| | | | GNRLCGPATAAWAGNWTGAPDVSALGAQGVLLLSTRDLAF |
| | | | AGAVEFLGLLAGACDRRLIVVNAVRAADWPADGPVVSRQH |
| | | | AYLACEVLPAVQCAVRWPAARDLRRTVLASGRVFGPGVFA |
| | | | RVEAAHARLYPDAPPLRLCRGANVRYRVRTRFGPDTLVPM |
| | | | SPREYRRAVLPALDGRAAASGAGDAMAPGAPDFCEDEAHS |
| | | | HRACARWGLGAPLRPVYVALGRDAVRGGPAELRGPRREFC |
| | | | ARALLEPDGDAPPLVLRDDADAGPPPQIRWASATGRAGTV |
| | | | LAAAGGGVEVVGTAAGLATPPRREPVDMDAELEDDDDGLF |
| | | | GE |
| 33 | RS1 | MS | MSAEQRKKKKTTTTQGRGAEVAMADEDGGRLRAAAETTGG |
| | [ICP4] | | PGSPDPADGPPPTPNPDRRPAARPGFGWHGGPEENEDEAD |
| | | | GAAADADADEAAPASGEAVDEPAADGVVSPRQLALLASMV |
| | | | DEAVRTIPSPPPERDGAEEEAARSPSPPRTPSMRADYGEE |
| | | | NDDDDDDDDDDDRDAGRWVRGPETTSAVRGAYPDPMASLS |
| | | | PRPPAPRRHHHHHHHRRRRAPRRRSAASDSSKSGSSSSAS |
| | | | SASSSASSSSSASASSSDDDDDDAARAPASAADHAADHAA |
| | | | DHAADHAADHAADHAAGGTLGADDEEAGVPARAPGAAPRP |
| | | | SPPRAEPAPGAGAGSARTPAATAGRLERRRARAAVAGRDA |
| | | | TGRFTAGRPRRVELDADAASGAFYARYRDGYVSGEPWPGA |
| | | | GPPPPGRVLYGGLGDSRPGLWGAPEAEEARARFEASGAPA |
| | | | PVWAPELGDAAQQYALITRLLYTPDAEAMGWLQNPRVAPG |
| | | | DVALDQACFRISGAARNSSSFISGSVARAVPHLGYAMAAG |
| | | | RFGWGLAHVAAAVAMSRRYDRAQKGFLLTSLRRAYAPLLA |
| | | | RENAALTGARTPDDGDDARGKPAAAAPLPSAAASPADERA |
| | | | VPAGYGAAGVLAALGRLSAAPASAPAGADDDDDDDGAGGG |
| | | | GGGGRRAEAGRVAVECLAACRGILEALAEGFDGDLAAVPG |
| | | | LAGARPAAPPRPGPAGAAAPPHADAPRLRAWLRELRFVRD |
| | | | ALVLMRLRGDLRVAGGSEAAVAAVRAVSLVAGALGPALPR |
| | | | SPRLLSSAAAAAADLLFQNQSLRPLLADTVAAADSLAAPA |
| | | | SAPREARKRKSPAPARAPPGGAPRPPKKSRADAPRPAAAP |
| | | | PAGAAPPAPPTPPPRPAALTRRPAEGPDPQGGWRRQPPGP |
| | | | SHTPAPSAAALEAYCAPRAVAELTDHPLFPAPWRPALMFD |
| | | | PRALASLAARCAAPPPGGAPAAFGPLRASGPLRRAAAWMR |
| | | | QVPDPEDVRVVILYSPLPGEDLAAGRAGGGPPPEWSAERG |
| | | | GLSCLLAALGNRLCGPATAAWAGNWTGAPDVSALGAQGVL |
| | | | LLSTRDLAFAGAVEFLGLLAGACDRRLIVVNAVRAADWPA |
| | | | DGPVVSRQHAYLACEVLPAVQCAVRWPAARDLRRTVLASG |
| | | | RVFGPGVFARVEAAHARLYPDAPPLRLCRGANVRYRVRTR |
| | | | FGPDTLVPMSPREYRRAVLPALDGRAAASGAGDAMAPGAP |
| | | | DFCEDEAHSHRACARWGLGAPLRPVYVALGRDAVRGGPAE |
| | | | LRGPRREFCARALLEPDGDAPPLVLRDDADAGPPPQIRWA |
| | | | SAAGRAGTVLAAAGGGVEVVGAAAGLATPPRREPVDMDAE |
| | | | LEDDDDGLFGE |
| 34 | RL2 | HG52 | MEPRPGTSSRADPGPERPPRQTPGTQPAAPHAWGMLNDMQ |
| | [ICP0] | | WLASSDSEEETEVGISDDDLHRDSTSEAGSTDTEMFEAGL |
| | | | MDAATPPARPPAERQGSPTPADAQGSCGGGPVGEEEAEAG |
| | | | GGGDVCAVCTDEIAPPLRCQSFPCLHPFCIPCMKTWIPLR |
| | | | NTCPLCNTPVAYLIVGVTASGSFSTIPIVNDPRTRVEAEA |
| | | | AVRAGTAVDFIWTGNPRTAPRSLSLGGHTVRALSPTPPWP |
| | | | GTDDEDDDLADVDYVPPAPRRAPRRGGGGAGATRGTSQPA |
| | | | ATRPAPPGAPRSSSSGGAPLRAGVGSGSGGGPAVAAVVPR |
| | | | VASLPPAAGGGRAQARRVGEDAAAAEGRTPPARQPRAAQE |
| | | | PPIVISDSPPPSPRRPAGPGPLSFVSSSSAQVSSGPGGGG |
| | | | LPQSSGRAARPRAAVAPRVRSPPRAAAAPVVSASADAAGP |
| | | | APPAVPVDAHRAPRSRMTQAQTDTQAQSLGRAGATDARGS |
| | | | GGPGAEGGPGVPRGTNTPGAAPHAAEGAAARPRKRRGSDS |
| | | | GPAASSSASSSAAPRSPLAPQGVGAKRAAPRRAPDSDSGD |
| | | | RGHGPLAPASAGAAPPSASPSSQAAVAAASSSSASSSSAS |
| | | | SSSASSSSASSSSASSSSASSSSASSSAGGAGGSVASASG |
| | | | AGERRETSLGPRAAAPRGPRKCARKTRHAEGGPEPGARDP |
| | | | APGLTRYLPIAGVSSVVALAPYVNKTVTGDCLPVLDMETG |
| | | | HIGAYVVLVDQTGNVADLLRAAAPAWSRRTLLPEHARNCV |
| | | | RPPDYPTPPASEWNSLWMTPVGNMLFDQGTLVGALDFHGL |
| | | | RSRHPWSREQGAPAPAGDAPAGHGE |
| 35 | RL2 | 333 | MEPRPGTSSRADPGPERPPRQTPGTQPAAPHAWGMLNDMQ |
| | [ICP0] | | WLASSDSEEETEVGISDDDLHRDSTSEAGSTDTEMFEAGL |
| | | | MDAATPPARPPAERQGSPTPADAQGSCGGGPVGEEEAEAG |
| | | | GGGDVCAVCTDEIAPPLRCQSFPCLHPFCIPCMKTWIPLR |
| | | | NTCPLCNTPVAYLIVGVTASGSFSTIPIVNDPRTRVEAEA |
| | | | AVRSGTAVDFIWTGNPRTAPRSLSLGGHTVRALSPTPPWP |
| | | | GTDDEDDDLADADYVPPAPRRAPRRGGGGAGATRGTSQPA |
| | | | ATRPAPPGAPRSSSSGGAPLRAGVGSGSGGGPAVAAVVPR |
| | | | VASLPPAAGGGRAQARRVGEDAAAAEGRTPPARQPRAAQE |
| | | | PPIVISDSPPPSPRRPAGPGPLSFVSSSSAQVSSGPGGGG |
| | | | LPQSSGRAARPRAAVAPRVRSPPRAAAAPVVSASADAAGP |
| | | | APPAVPVDAHRAPRSRMTQAQTDTQAQSLGRAGATDARGS |
| | | | GGPGAEGGPGVPRGTNTPGAAPHAAEGAAARPRKRRGSDS |
| | | | GPAASSSASSSAAPRSPLAPQGVGAKRAAPRRAPDSDSGG |
| | | | RGHGPLAPASAGAAPPSASPSSQAAVAAAAASSSSSSSAS |
| | | | SSSSSASSSSSSASSSAGGAGGSVASASGAGERRETSLGP |
| | | | RAAAPRGPRKCARKTRHAEGGPEPGARDPAPGLTRYLPIA |
| | | | GVSSVVALAPYVNKTVTGDCLPVLDMETGHIGAYVVLVDQ |
| | | | TGNVADLLRAAAPAWSRRTLLPEHARNCVRPPDYPTPPAS |
| | | | EWNSLWMTPVGNMLFDQGTLVGALDFHGLRSRHPWSREQG |
| | | | APAPAGDAPAGHGE |
| 36 | RL2 | MS | MEPRPGTSSRADPGPERPPRQTPGTQPAAPHAWGMLNDMQ |
| | [ICP0] | | WLASSDSEEETEVGISDDDLHRDSTSEAGSTDTEMFEAGL |
| | | | MDAATPPARPPAERQGSPTPADAQGSCGGGPVGEEEAEAG |
| | | | GGGDVCAVCTDEIAPPLRCQSFPCLHPFCIPCMKTWIPLR |
| | | | NTCPLCNTPVAYLIVGVTASGSFSTIPIVNDPRTRVEAEA |
| | | | AVRSGTAVDFIWTGNPRTAPRSLSLGGHTVRALSPTPPWP |
| | | | GTDDEDDDLADADYVPPAPRRAPRRGGGGAGATRGTSQPA |
| | | | ATRPAPPGAPRSSSSGGAPLRAGVGSGSGGGPAVAAVVPR |
| | | | VASLPPAAGGGRAQARRVGEDAAAAEGRTPPARQPRAAQE |
| | | | PPIVISDSPPPSPRRPAGPGPLSFFSSSSAQVSSGPGGGG |
| | | | LPQSSGRAARPRAAVAPRVRSPPRAAAAPVVSASADAAGP |
| | | | APPAVPVDAHRAPRSRMTQAQTDTQAQSLGRAGATDARGS |
| | | | GGPGAEGGPGVPRGTNTPGAAPHAAEGAAARPRKRRGSDS |
| | | | GPAASSSASSSAAPRSPLAPQGVGAKRAAPRRAPDSDSGD |
| | | | RGHGPLAPASAGAAPPSASPSSQAAVAAAASSSSSSSSSA |
| | | | SSSAGGAGGSVASASGAGERRETSLGPRAAAPRGPRKCAR |
| | | | KTRHAEGGPEPGARDPAPGLTRYLPIAGASSVVALAPYVN |
| | | | KTVTGDCLPILDMETGHIGAYVVLVDQTGNVADLLRAAAP |
| | | | AWSRRTLLPEHARNCVRPPDYPTPPASEWNSLWMTPVGNM |
| | | | LFDQGTLVGALDFHGLRSRHPWSREQGAPAPAGDAPAGHG |
| | | | E |
| 37 | UL5 | HG52 | MAASGGEGSRDVRAPGPPPQQPGARPAVRFRDEAFLNFTS |
| | | | MHGVQPIIARIRELSQQQLDVTQVPRLQWFRDVAALEVPT |
| | | | GLPLREFPFAAYLITGNAGSGKSTCVQTLNEVLDCVVTGA |
| | | | TRIAAQNMYVKLSGAFLSRPINTIFHEFGFRGNHVQAQLG |
| | | | QHPYTLASSPASLEDLQRRDLTYYWEVILDITKRALAAHG |
| | | | GEDARNEFHALTALEQTLGLGQGALTRLASVTHGALPAFT |
| | | | RSNIIVIDEAGLLGRHLLTTVVYCWWMINALYHTPQYAGR |
| | | | LRPVLVCVGSPTQTASLESTFEHQKLRCSVRQSENVLTYL |
| | | | ICNRTLREYTRLSHSWAIFINNKRCVEHEFGNLMKVLEYG |
| | | | LPITEEHMQFVDRFVVPESYITNPANLPGWTRLFSSHKEV |
| | | | SAYMAKLHAYLKVTREGEFVVFTLPVLTFVSVKEFDEYRR |
| | | | LTQQPTLTMEKWITANASRITNYSQSQDQDAGHVRCEVHS |
| | | | KQQLVVARNDITYVLNSQVAVTARLRKMVFGFDGTFRTFE |
| | | | AVLRDDSFVKTQGETSVEFAYRFLSRLMFGGLIHFYNFLQ |
| | | | RPGLDATQRTLAYGRLGELTAELLSLRRDAAGASATRAAD |
| | | | TSDRSPGERAFNFKHLGPRDGGPDDFPDDDLDVIFAGLDE |
| | | | QQLDVFYCHYALEEPETTAAVHAQFGLLKRAFLGRYLILR |
| | | | ELFGEVFESAPFSTYVDNVIFRGCELLTGSPRGGLMSVAL |
| | | | QTDNYTLMGYTYTRVFAFAEELRRRHATAGVAEFLEESPL |
| | | | PYIVLRDQHGFMSVVNTNISEFVESIDSTELAMAINADYG |
| | | | ISSKLAMTITRSQGLSLDKVAICFTPGNLRLNSAYVAMSR |
| | | | TTSSEFLHMNLNPLRERHERDDVISEHILSALRDPNVVIV |
| | | | Y |
| 38 | UL5 | 333 | MAASGGEGSRDVRAPGPPPQQPGARPAVRFRDEAFLNFTS |
| | | | MHGVQPIIARIRELSQQQLDVTQVPRLQWFRDVAALEVPT |
| | | | GLPLREFPFAAYLITGNAGSGKSTCVQTLNEVLDCVVTGA |
| | | | TRIAAQNMYVKLSGAFLSRPINTIFHEFGFRGNHVQAQLG |
| | | | QHPYTLASSPASLEDLQRRDLTYYWEVILDITKRALAAHG |
| | | | GEDARNEFHALTALEQTLGLGQGALTRLASVTHGALPAFT |
| | | | RSNIIVIDEAGLLGRHLLTTVVYCWWMINALYHTPQYAGR |
| | | | LRPVLVCVGSPTQTASLESTFEHQKLRCSVRQSENVLTYL |
| | | | ICNRTLREYTRLSHSWAIFINNKRCVEHEFGNLMKVLEYG |
| | | | LPITEEHMQFVDRFVVPESYITNPANLPGWTRLFSSHKEV |
| | | | SAYMAKLHAYLKVTREGEFVVFTLPVLTFVSVKEFDEYRR |
| | | | LTQQPTLTMEKWITANASRITNYSQSQDQDAGHVRCEVHS |
| | | | KQQLVVARNDITYVLNSQVAVTARLRKMVFGFDGTFRTFE |
| | | | AVLRDDSFVKTQGETSVEFAYRFLSRLMFGGLIHFYNFLQ |
| | | | RPGLDATQRTLAYGRLGELTAELLSLRRDAAGASATRAAD |
| | | | TSDRSPGERAFNFKHLGPRDGGPDDFPDDDLDVIFAGLDE |
| | | | QQLDVFYCHYALEEPETTAAVHAQFGLLKRAFLGRYLILR |
| | | | ELFGEVFESAPFSTYVDNVIFRGCELLTGSPRGGLMSVAL |
| | | | QTDNYTLMGYTYTRVFAFAEELRRRHATAGVAEFLEESPL |
| | | | PYIVLRDQHGFMSVVNTNISEFVESIDSTELAMAINADYG |
| | | | ISSKLAMTITRSQGLSLDKVAICFTPGNLRLNSAYVAMSR |
| | | | TTSSEFLHMNLNPLRERHERDDVISEHILSALRDPNVVIV |
| | | | Y |
| 39 | UL5 | MS | MAASGGEGSRDVRAPGPPPQQPGARPAVRFRDEAFLNFTS |
| | | | MHGVQPIIARIRELSQQQLDVTQVPRLQWFRDVAALEVPT |
| | | | GLPLREFPFAAYLITGNAGSGKSTCVQTLNEVLDCVVTGA |
| | | | TRIAAQNMYVKLSGAFLSRPINTIFHEFGFRGNHVQAQLG |
| | | | QHPYTLASSPASLEDLQRRDLTYYWEVILDITKRALAAHG |
| | | | GEDARNEFHALTALEQTLGLGQGALTRLASVTHGALPAFT |
| | | | RSNIIVIDEAGLLGRHLLTTVVYCWWMINALYHTPQYAGR |
| | | | LRPVLVCVGSPTQTASLESTFEHQKLRCSVRQSENVLTYL |
| | | | ICNRTLREYTRLSHSWAIFINNKRCVEHEFGNLMKVLEYG |
| | | | LPITEEHMQFVDRFVVPESYITNPANLPGWTRLFSSHKEV |
| | | | SAYMAKLHAYLKVTREGEFVVFTLPVLTFVSVKEFDEYRR |
| | | | LTQQPTLTMEKWITANASRITNYSQSQDQDAGHVRCEVHS |
| | | | KQQLVVARNDITYVLNSQVAVTARLRKMVFGFDGTFRTFE |
| | | | AVLRDDSFVKTQGETSVEFAYRFLSRLMFGGLIHFYNFLQ |
| | | | RPGLDATQRTLAYGRLGELTAELLSLRRDAAGASATRAAD |
| | | | TSDRSPGERAFNFKHLGPRDGGPDDFPDDDLDVIFAGLDE |
| | | | QQLDVFYCHYALEEPETTAAVHAQFGLLKRAFLGRYLILR |
| | | | ELFGEVFESAPFSTYVDNVIFRGCELLTGSPRGGLMSVAL |
| | | | QTDNYTLMGYTYTRVFAFAEELRRRHATAGVAEFLEESPL |
| | | | PYIVLRDQHGFMSVVNTNISEFVESIDSTELAMAINADYG |
| | | | ISSKLAMTITRSQGLSLDKVAICFTPGNLRLNSAYVAMSR |
| | | | TTSSEFLHMNLNPLRERHERDDVISEHILSALRDPNVVIV |
| | | | Y |
| 40 | UL9 | HG52 | MNVATCTHQTHHAARAPGATSAPGAASGDPLGARRPIGDD |
| | | | ECEQYTSSVSLARMLYGGDLAEWVPRVHPKTTIERQQHGP |
| | | | VTFPDASAPTARCVTVVRAPMGSGKTTALIRWLGEAIHSP |
| | | | DTSVLVVSCRRSFTQTLATRFAESGLPDFVTYFSSTNYIM |
| | | | NDRPFHRLIVQVESLHRVGPNLLNNYDVLVLDEVMSTLGQ |
| | | | LYSPTMQQLGRVDALMLRLLRTCPRIIAMDATANAQLVDF |
| | | | LCSLRGEKNVHVVIGEYAMPGFSARRCLFLPRLGPEVLQA |
| | | | ALRRRGPAGGAPPPDAPPDATFFGELEARLAGGDNVCIFS |
| | | | STVSFAEVVARFCRQFTDRVLLLHSLTPPGDVTTWGRYRV |
| | | | VIYTTVVTVGLSFDPPHFDSMFAYVKPMNYGPDMVSVYQS |
| | | | LGRVRTLRKGELLIYMDGSGARSEPVFTPMLLNHVVSASG |
| | | | QWPAQFSQVTNLLCRRFKGRCDASHADAAQARGSRIYSKF |
| | | | RYKHYFERCTLACLADSLNILHMLLTLNCMHVRFWGHDAA |
| | | | LTPRNFCLFLRGIHFDALRAQRDLRELRCQDPDTSLSAQA |
| | | | AETEEVGLFVEKYLRPDVAPAEVVALMRGLNSLVGRTRFI |
| | | | YLVLLEACLRVPMAAHSSAIFRRLYDHYATGVIPTINAAG |
| | | | ELELVALHPTLNVAPVWELFRLCSTMAACLQWDSMAGGSG |
| | | | RTFSPEDVLELLNPHYDRYMQLVFELGHCNVTDGPLLSED |
| | | | AVKRVADALSGCPPRGSVSETEHALSLFKIIWGELFGVQL |
| | | | AKSTQTFPGAGRVKNLTKRAIVELLDAHRIDHSACRTHRQ |
| | | | LYALLMAHKREFAGARFKLRAPAWGRCLRTHASGAQPNTD |
| | | | IILEAALSELPTEAWPMMQGAVNESTL |
| 41 | UL9 | 333 | MAETMNVATCTHQTHHAARAPGATSAPGAASGDPPGARRP |
| | | | IGDDECEQYTSSVSLARMLYGGDLAEWVPRVHPKTTIERQ |
| | | | QHGPVTFPDASAPTARCVTVVRAPMGSGKTTALIRWLGEA |
| | | | IHSPDTSVLVVSCRRSFTQTLATRFAESGLPDFVTYFSST |
| | | | NYIMNDRPFHRLIVQVESLHRVGPNLLNNYDVLVLDEVMS |
| | | | TLGQLYSPTMQQLGRVDALMLRLLRTCPRIIAMDATANAQ |
| | | | LVDFLCSLRGEKNVHVVIGEYAMPGFSARRCLFLPRLGPE |
| | | | VLQAALRPPGPAGGAPPPDAPPDATFFGELEARLAGGDNV |
| | | | CIFSSTVSFAEVVARFCRQFTDRVLLLHSLTPPGDVTTWG |
| | | | RYRVVIYTTVVTVGLSFDPPHFDSMFAYVKPMNYGPDMVS |
| | | | VYQSLGRVRTLRKGELLIYMDGSGARSEPVFTPMLLNHVV |
| | | | SASGQWPAQFSQVTNLLCRRFKGRCDASHADAAQARGSRI |
| | | | YSKFRYKHYFERCTLACLADSLNILHMLLTLNCMHVRFWG |
| | | | HDAALTPRNFCLFLRGIHFDALRAQRDLRELRCQDPDTSL |
| | | | SAQAAETEEVGLFVEKYLRPDVAPAEVVALMRGLNSLVGR |
| | | | TRFIYLVLLEACLRVPMAAHSSAIFRRLYDHYATGVIPTI |
| | | | NAAGELELVALHPTLNVAPVWELFRLCSTMAACLQWDSMA |
| | | | GGSGRTFSPEDVLELLNPHYDRYMQLVFELGHCNVTDGPL |
| | | | LSEDAVKRVADALSGCPPRGSVSETEHALSLFKIIWGELF |
| | | | GVQLAKSTQTFPGAGRVKNLTKRAIVELLDAHRIDHSACR |
| | | | THRQLYALLMAHKREFAGARFKLRAPAWGRCLRTHASGAQ |
| | | | PNTDIILEAALSELPTEAWPMMQGAVNESTL |
| 42 | UL9 | MS | MAETMNVATCTHQTHHAARAPGATSAPGAASGDPLGARRP |
| | | | IGDDECEQYTSSVSLARMLYGGDLAEWVPRVHPKTTIERQ |
| | | | QHGPVTFPDASAPTARCVTVVRAPMGSGKTTALIRWLGEA |
| | | | IHSPDTSVLVVSCRRSFTQTLATRFAESGLPDFVTYFSST |
| | | | NYIMNDRPFHRLIVQVESLHRVGPNLLNNYDVLVLDEVMS |
| | | | TLGQLYSPTMQQLGRVDALMLRLLRTCPRIIAMDATANAQ |
| | | | LVDFLCSLRGEKNVHVVIGEYAMPGFSARRCLFLPRLGPE |
| | | | VLQAALRPPGPAGGAPPPDAPPDATFFGELEARLAGGDNV |
| | | | CIFSSTVSFAEVVARFCRQFTDRVLLLHSLTPPGDVTTWG |
| | | | RYRVVIYTTVVTVGLSFDPPHFDSMFAYVKPMNYGPDMVS |
| | | | VYQSLGRVRTLRKGELLIYMDGSGARSEPVFTPMLLNHVV |
| | | | SASGQWPAQFSQVTNLLCRRFKGRCDASRADAAQARGSRI |
| | | | YSKFRYKHYFERCTLACLADSLNILHMLLTLNCMHVRFWG |
| | | | HDAALTPRNFCLFLRGIHFDALRAQRDLRELRCQDPDTSL |
| | | | SAQAAETEEVGLFVEKYLRPDVAPAEVVALMRGLNSLVGR |
| | | | TRFIYLVLLEACLRVPMAAHSSAIFRRLYDHYATGVIPTI |
| | | | NAAGELELVALHPTLNVAPVWELFRLCSTMAACLQWDSMA |
| | | | GGSGRTFSPEDVLELLNPHYDRYMQLVFELGHCNVTDGPL |
| | | | LSEDAVKRVADALSGCPPRGSVSETEHALSLFKIIWGELF |
| | | | GVQLAKSTQTFPGAGRVKNLTKRAIVELLDAHRIDHSACR |
| | | | THRQLYALLMAHKREFAGARFKLRAPAWGRCLRTHASGAQ |
| | | | PNTDIILEAALSELPTEAWPMMQGAVNESTL |
| 43 | UL19 | HG52 | MAAPARDPPGYRYAAAILPTGSILSTIEVASHRRLFDFFA |
| | | | AVRSDENSLYDVEFDALLGSYCNTLSLVRFLELGLSVACV |
| | | | CTKFPELAYMNEGRVQFEVHQPLIARDGPHPVEQPVHNYM |
| | | | TKVIDRRALNAAFSLATEAIALLTGEALDGTGISLHRQLR |
| | | | AIQQLARNVQAVLGAFERGTADQMLHVLLEKAPPLALLLP |
| | | | MQRYLDNGRLATRVARATLVAELKRSFCDTSFFLGKAGHR |
| | | | REAIEAWLVDLTTATQPSVAVPRLTHADTRGRPVDGVLVT |
| | | | TAAIKQRLLQSFLKVEDTEADVPVTYGEMVLNGANLVTAL |
| | | | VMGKAVRSLDDVGRHLLDMQEEQLEANRETLDELESAPQT |
| | | | TRVRADLVAIGDRLVFLEALERRIYAATNVPYPLVGAMDL |
| | | | TFVLPLGLENPAMERFAAHAGDLVPAPGHPEPRAFPPRQL |
| | | | FFWGKDHQVLRLSMENAVGTVCHPSLMNIDAAVGGVNHDP |
| | | | VEAANPYGAYVAAPAGPGADMQQRFLNAWRQRLAHGRVRW |
| | | | VAECQMTAEQFMQPDNANLALELHPAFDFFAGVADVELPG |
| | | | GEVPPAGPGAIQATWRVVNGNLPLALCPVAFRDARGLELG |
| | | | VGRHAMAPATIAAVRGAFEDRSYPAVFYLLQAAIHGNEHV |
| | | | FCALARLVTQCITSYWNNTRCAAFVNDYSLVSYIVTYLGG |
| | | | DLPEECMAVYRDLVAHVEALAQLVDDFTLPGPELGGQAQA |
| | | | ELNHLMRDPALLPPLVWDCDGLMRHAALDRHRDCRIDAGG |
| | | | HEPVYAAACNVATADFNRNDGRLLHNTQARAADAADDRPH |
| | | | RPADWTVHHKIYYYVLVPAFSRGRCCTAGVRFDRVYATLQ |
| | | | NMVVPEIAPGEECPSDPVTDPAHPLHPANLVANTVKRMFH |
| | | | NGRVVVDGPAMLTLQVLAHNMAERTTALLCSAAPDAGANT |
| | | | ASTANMRIFDGALHAGVLLMAPQHLDHTIQNGEYFYVLPV |
| | | | HALFAGADHVANAPNFPPALRDLARDVPLVPPALGANYFS |
| | | | SIRQPVVQHARESAAGENALTYALMAGYFKMSPVALYHQL |
| | | | KTGLHPGFGFTVVRQDRFVTENVLFSERASEAYFLGQLQV |
| | | | ARHETGGGVNFTLTQPRGNVDLGVGYTAVAATGTVRNPVT |
| | | | DMGNLPQNFYLGRGAPPLLDNAAAVYLRNAVVAGNRLGPA |
| | | | QPLPVFGCAQVPRRAGMDHGQDAVCEFIATPVATDINYFR |
| | | | RPCNPRGRAAGGVYAGDKEGDVIALMYDHGQSDPARPFAA |
| | | | TANPWASQRFSYGDLLYNGAYHLNGASPVLSPCFKFFTAA |
| | | | DITAKHRCLERLIVETGSAVSTATAASDVQFKRPPGCREL |
| | | | VEDPCGLFQEAYPITCASDPALLRSARDGEAHARETHFTQ |
| | | | YLIYDASPLKGLSL |
| 44 | UL19 | 333 | MAAPARDPPGYRYAAAMVPTGSILSTIEVASHRRLFDFFA |
| | | | RVRSDENSLYDVEFDALLGSYCNTLSLVRFLELGLSVACV |
| | | | CTKFPELAYMNEGRVQFEVHQPLIARDGPHPVEQPVHNYM |
| | | | TKVIDRRALNAAFSLATEAIALLTGEALDGTGISLHRQLR |
| | | | AIQQLARNVQAVLGAFERGTADQMLHVLLEKAPPLALLLP |
| | | | MQRYLDNGRLATRVARATLVAELKRSFCDTSFFLGKAGHR |
| | | | REAIEAWLVDLTTATQPSVAVPRLTHADTRGRPVDGVLVT |
| | | | TAAIKQRLLQSFLKVEDTEADVPVTYGEMVLNGANLVTAL |
| | | | VMGKAVRSLDDVGRHLLEMQEEQLEANRETLDELESAPQT |
| | | | TRVRADLVAIGDRLVFLEALEKRIYAATNVPYPLVGAMDL |
| | | | TFVLPLGLENPAMERFAAHAGDLVPAPGHPEPRAFPPRQL |
| | | | FFWGKDHQVLRLSMENAVGTVCHPSLMNIDAAVGGVNHDP |
| | | | VEAANPYGAYVAAPAGPGADMQQRFLNAWRQRLAHGRVRW |
| | | | VAECQMTAEQFMQPDNANLALELHPAFDFFAGVADVELPG |
| | | | GEVPPAGPGAIQATWRVVNGNLPLALCPVAFRDARGLELG |
| | | | VGRHAMAPATIAAVRGAFEDRSYPAVFYLLQAAIHGSEHV |
| | | | FCALARLVTQCITSYWNNTRCAAFVNDYSLVSYIVTYLGG |
| | | | DLPEECMAVYRDLVAHVEALAQLVDDFTLPGPELGGQAQA |
| | | | ELNHLMRDPALLPPLVWDCDGLMRHAALDRHRDCRIDAGG |
| | | | HEPVYAAACNVATADENRNDGRLLHNTQARAADAADDRPH |
| | | | RPADWTVHHKIYYYVLVPAFSRGRCCTAGVRFDRVYATLQ |
| | | | NMVVPEIAPGEECPSDPVTDPAHPLHPANLVANTVNAMFH |
| | | | NGRVVVDGPAMLTLQVLAHNMAERTTALLCSAAPDAGANT |
| | | | ASTANMRIFDGALHAGVLLMAPQHLDHTIQNGEYFYVLPV |
| | | | HALFAGADHVANAPNFPPALRDLARHVPLVPPALGANYFS |
| | | | SIRQPVVQHARESAAGENALTYALMAGYFKMSPVALYHQL |
| | | | KTGLHPGFGFTVVRQDRFVTENVLFSERASEAYFLGQLQV |
| | | | ARHETGGGVSFTLTQPRGNVDLGVGYTAVAATATVRNPVT |
| | | | DMGNLPQNFYLGRGAPPLLDNAAAVYLRNAVVAGNRLGPA |
| | | | QPLPVFGCAQVPRRAGMDHGQDAVCEFIATPVATDINYFR |
| | | | RPCNPRGRAAGGVYAGDKEGDVIALMYDHGQSDPARPFAA |
| | | | TANPWASQRFSYGDLLYNGAYHLNGASPVLSPCFKFFTAA |
| | | | DITAKHRCLERLIVETGSAVSTATAASDVQFKRPPGCREL |
| | | | VEDPCGLFQEAYPITCASDPALLRSARDGEAHARETHFTQ |
| | | | YLIYDASPLKGLSL |
| 45 | UL19 | MS | MAAPARDPPGYRYAAAMVPTGSILSTIEVASHRRLFDFFA |
| | | | RVRSDENSLYDVEFDALLGSYCNTLSLVRFLELGLSVACV |
| | | | CTKFPELAYMNEGRVQFEVHQPLIARDGPHPVEQPVHNYM |
| | | | TKVIDRRALNAAFSLATEAIALLTGEALDGTGISLHRQLR |
| | | | AIQQLARNVQAVLGAFERGTADQMLHVLLEKAPPLALLLP |
| | | | MQRYLDNGRLATRVARATLVAELKRSFCDTSFFLGKAGHR |
| | | | REAIEAWLVDLTTATQPSVAVPRLTHADTRGRPVDGVLVT |
| | | | TAAIKQRLLQSFLKVEDTEADVPVTYGEMVLNGANLVTAL |
| | | | VMGKAVRSLDDVGRHLLEMQEEQLEANRETLDELESAPQT |
| | | | TRVRADLVAIGDRLVFLEALEKRIYAATNVPYPLVGAMDL |
| | | | TFVLPLGLFNPAMERFAAHAGDLVPAPGHPEPRAFPPRQL |
| | | | FFWGKDHQVLRLSMENAVGTVCHPSLMNIDAAVGGVNHAP |
| | | | VEAANPYGAYVAAPAGPGADMQQRFLNAWRQRLAHGRVRW |
| | | | VAECQMTAEQFMQPDNANLALELHPAFDFFAGVADVELPG |
| | | | GEVPPAGPGAIQATWRVVNGNLPLALCPVAFRDARGLELG |
| | | | VGRHAMAPATIAAVRGAFEDRSYPAVFYLLQAAIHGSEHV |
| | | | FCALARLVTQCITSYWNNTRCAAFVNDYSLVSYIVTYLGG |
| | | | DLPEECMAVYRDLVAHVEALAQLVDDFTLPGPELGGQAQA |
| | | | ELNHLMRDPALLPPLVWDCDGLMRHAALDRHRDCRIDAGG |
| | | | HEPVYAAACNVATADENRNDGRLLHNTQARAADAADDRPH |
| | | | RPADWTVHHKIYYYVLVPAFSRGRCCTAGVRFDRVYATLQ |
| | | | NMVVPEIAPGEECPSDPVTDPAHPLHPANLVANTVNAMFH |
| | | | NGRVVVDGPAMLTLQVLAHNMAERTTALLCSAAPDAGANT |
| | | | ASTANMRIFDGALHAGVLLMAPQHLDHTIQNGEYFYVLPV |
| | | | HALFAGADHVANAPNFPPALRDLARHVPLVPPALGANYFS |
| | | | SIRQPVVQHARESAAGENALTYALMAGYFKMSPVALYHQL |
| | | | KTGLHPGFGFTVVRQDRFVTENVLFSERASEAYFLGQLQV |
| | | | ARHETGGGVSFTLTQPRGNVDLGVGYTAVAATATVRNPVT |
| | | | DMGNLPQNFYLGRGAPPLLDNAAAVYLRNAVVAGNRLGPA |
| | | | QPLPVFGCAQVPRRAGMDHGQDAVCEFIATPVATDINYFR |
| | | | RPCNPRGRAAGGVYAGDKEGDVIALMYDHGQSDPARPFAA |
| | | | TANPWASQRFSYGDLLYNGAYHLNGASPVLSPCFKFFTAA |
| | | | DITAKHRCLERLIVETGSAVSTATAASDVQFKRPPGCREL |
| | | | VEDPCGLFQEAYPITCASDPALLRSARDGEAHARETHFTQ |
| | | | YLIYDASPLKGLSL |
| 46 | UL25 | HG52 | MDPYYPFDALDVWEHRRFIVADSRSFITPEFPRDFWMLPV |
| | | | ENIPRETAAERAAVLQAQRTAAAAALENAALQAAELPVDI |
| | | | ERRIRPIEQQVHHIADALEALETAAAAAEEADAARDAEAR |
| | | | GEGAADGAAPSPTAGPAAAEMEVQIVRNDPPLRYDTNLPV |
| | | | DLLHMVYAGRGAAGSSGVVFGTWYRTIQERTIADFPLTTR |
| | | | SADFRDGRMSKTFMTALVLSLQSCGRLYVGQRHYSAFECA |
| | | | VLCLYLLYRTTHESSPDRDRAPVAFGDLLARLPRYLARLA |
| | | | AVIGDESGRPQYRYRDDKLPKAQFAAAGGRYEHGALATHV |
| | | | VIATLVRHGVLPAAPGDVPRDTSTRVNPDDVAHRDDVNRA |
| | | | AAAFLARGHNLFLWEDQTLLRATANTITALAVLRRLLANG |
| | | | NVYADRLDNRLQLGMLIPGAVPAEAIARGASGLDSGAIKS |
| | | | GDNNLEALCVNYVLPLYQADPTVELTQLFPGLAALCLDAQ |
| | | | AGRPLASTRRVVDMSSGARQAALVRLTALELINRTRTNTT |
| | | | PVGEIINAHDALGIQYEQGPGLLAQQARIGLASNTKRFAT |
| | | | FNVGSDYDLLYFLCLGFIPQYLSVA |
| 47 | UL25 | 333 | MDPYYPFDALDVWEHRRFIVADSRSFITPEFPRDFWMLPV |
| | | | FNIPRETAAERAAVLQAQRTAAAAALENAALQAAELPVDI |
| | | | ERRIRPIEQQVHHIADALEALETAAAAAEEADAARDAEAR |
| | | | GEGAADGAAPSPTAGPAAAEMEVQIVRNDPPLRYDTNLPV |
| | | | DLLHMVYAGRGAAGSSGVVFGTWYRTIQERTIADFPLTTR |
| | | | SADFRDGRMSKTFMTALVLSLQSCGRLYVGQRHYSAFECA |
| | | | VLCLYLLYRTTHESSPDRDRAPVAFGDLLARLPRYLARLA |
| | | | AVIGDESGRPQYRYRDDKLPKAQFAAAGGRYEHGALATHV |
| | | | VIATLVRHGVLPAAPGDVPRDTSTRVNPDDVAHRDDVNRA |
| | | | AAAFLARGHNLFLWEDQTLLRATANTITALAVLRRLLANG |
| | | | NVYADRLDNRLQLGMLIPGAVPAEAIARGASGLDSGAIKS |
| | | | GDNNLEALCVNYVLPLYQADPTVELTQLFPGLAALCLDAQ |
| | | | AGRPLASTRRVVDMSSGARQAALVRLTALELINRTRTNTT |
| | | | PVGEIINAHDALGIQYEQGLGLLAQQARIGLASNAKRFAT |
| | | | ENVGSDYDLLYFLCLGFIPQYLSVA |
| 48 | UL25 | MS | MDPYYPFDALDVWEHRRFIVADSRSFITPEFPRDFWMLPV |
| | | | FNIPRETAAERAAVLQAQRTAAAAALENAALQAAELPVDI |
| | | | ERRIRPIEQQVHHIADALEALETAAAAAEEADAARDAEAR |
| | | | GEGAADGAAPSPTAGPAAAEMEVQIVRNDPPLRYDTNLPV |
| | | | DLLHMVYAGRGAAGSSGVVFGTWYRTIQERTIADFPLTTR |
| | | | SADFRDGRMSKTFMTALVLSLQSCGRLYVGQRHYSAFECA |
| | | | VLCLYLLYRTTHESSPDRDRAPVAFGDLLARLPCYLARLA |
| | | | AVIGDESGRPQYRYRDDKLPKAQFAAAGGRYEHGALATHV |
| | | | VIATLVRHGVLPAAPGDVPRDTSTRVNPDDVAHRDDVNRA |
| | | | AAAFLARGHNLFLWEDQTLLRATANTITALAVLRRLLANG |
| | | | NVYADRLDNRLQLGMLIPGAVPAEAIARGASGLDSGAIKS |
| | | | GDNNLEALCVNYVLPLYQADPTVELTQLFPGLAALCLDAQ |
| | | | AGRPLASTRRVVDMSSGARQAALVRLTALELINRTRTNTT |
| | | | PVGEIINAHDALGIQYEQGLGLLAQQARIGLASNAKRFAT |
| | | | ENVGSDYDLLYFLCLGFIPQYLSVA |
| 49 | UL30 | HG52 | MFCAAGGPASPGGKPAARAASGFFAPHNPRGATQTAPPPC |
| | | | RRQNFYNPHLAQTGTQPKALGPAQRHTYYSECDEFRFIAP |
| | | | RSLDEDAPAEQRTGVHDGRLRRAPKVYCGGDERDVLRVGP |
| | | | EGFWPRRLRLWGGADHAPEGFDPTVTVFHVYDILEHVEHA |
| | | | YSMRAAQLHERFMDAITPAGTVITLLGLTPEGHRVAVHVY |
| | | | GTRQYFYMNKAEVDRHLQCRAPRDLCERLAAALRESPGAS |
| | | | FRGISADHFEAEVVERADVYYYETRPTLYYRVFVRSGRAL |
| | | | AYLCDNFCPAIRKYEGGVDATTRFILDNPGFVTFGWYRLK |
| | | | PGRGNAPAQPRPPTAFGTSSDVEFNCTADNLAVEGAMCDL |
| | | | PAYKLMCFDIECKAGGEDELAFPVAERPEDLVIQISCLLY |
| | | | DLSTTALEHILLESLGSCDLPESHLSDLASRGLPAPVVLE |
| | | | FDSEFEMLLAFMTFVKQYGPEFVTGYNIINFDWPFVLTKL |
| | | | TEIYKVPLDGYGRMNGRGVFRVWDIGQSHFQKRSKIKVNG |
| | | | MVNIDMYGIITDKVKLSSYKLNAVAEAVLKDKKKDLSYRD |
| | | | IPAYYASGPAQRGVIGEYCVQDSLLVGQLFFKFLPHLELS |
| | | | AVARLAGINITRTIYDGQQIRVFTCLLRLAGQKGFILPDT |
| | | | QGRFRGLDKEAPKRPAVPRGEGERPGDGNGDEDKDDDEDG |
| | | | DEDGDEREEVARETGGRHVGYQGARVLDPTSGFHVDPVVV |
| | | | FDFASLYPSIIQAHNLCESTLSLRPEAVAHLEADRDYLEI |
| | | | EVGGRRLFFVKAHVRESLLSILLRDWLAMRKQIRSRIPQS |
| | | | TPEEAVLLDKQQAAIKVVCNSVYGFTGVQHGLLPCLHVAA |
| | | | TVTTIGREMLLATRAYVHARWAEFDQLLADFPEAAGMRAP |
| | | | GPYSMRIIYGDTDSIFVLCRGLTAAGLVAMGDKMASHISR |
| | | | ALFLPPIKLECEKTFTKLLLIAKKKYIGVICGGKMLIKGV |
| | | | DLVRKNNCAFINRTSRALVDLLFYDDTVSGAAAALAERPA |
| | | | EEWLARPLPEGLQAFGAVLVDAHRRITDPERDIQDFVLTA |
| | | | ELSRHPRAYTNKRLAHLTVYYKLMARRAQVPSIKDRIPYV |
| | | | IVAQTREVEETVARLAALRELDAAAPGDEPAPPAALPSPA |
| | | | KRPRETPSHADPPGGASKPRKLLVSELAEDPGYAIARGVP |
| | | | LNTDYYFSHLLGAACVTFKALFGNNAKITESLLKRFIPET |
| | | | WHPPDDVAARLRAAGFGPAGAGATAEETRRMLHRAFDTLA |
| 50 | UL30 | 333 | MFCAAGGPTSPGGKSAARAASGFFAPHNPRGATQTAPPPC |
| | | | RRQNFYNPHLAQTGTQPKAPGPAQRHTYYSECDEFRFIAP |
| | | | RSLDEDAPAEQRTGVHDGRLRRAPKVYCGGDERDVLRVGP |
| | | | EGFWPRRLRLWGGADHAPEGFDPTVTVFHVYDILEHVEHA |
| | | | YSMRAAQLHERFMDAITPAGTVITLLGLTPEGHRVAVHVY |
| | | | GTRQYFYMNKAEVDRHLQCRAPRDLCERLAAALRESPGAS |
| | | | FRGISADHFEAEVVERADVYYYETRPTLYYRVFVRSGRAL |
| | | | AYLCDNFCPAIRKYEGGVDATTRFILDNPGFVTFGWYRLK |
| | | | PGRGNAPAQPRPPTAFGTSSDVEFNCTADNLAVEGAMCDL |
| | | | PAYKLMCFDIECKAGGEDELAFPVAERPEDLVIQISCLLY |
| | | | DLSTTALEHILLESLGSCDLPESHLSDLASRGLPAPVVLE |
| | | | FDSEFEMLLAFMTFVKQYGPEFVTGYNIINFDWPFVLTKL |
| | | | TEIYKVPLDGYGRMNGRGVFRVWDIGQSHFQKRSKIKVNG |
| | | | MVNIDMYGIITDKVKLSSYKLNAVAEAVLKDKKKDLSYRD |
| | | | IPAYYASGPAQRGVIGEYCVQDSLLVGQLFFKFLPHLELS |
| | | | AVARLAGINITRTIYDGQQIRVFTCLLRLAGQKGFILPDT |
| | | | QGRFRGLDKEAPKRPAVPRGEGERPGDGNGDEDKDDDEDG |
| | | | DEDGDEREEVARETGGRHVGYQGARVLDPTSGFHVDPVVV |
| | | | FDFASLYPSIIQAHNLCESTLSLRPEAVAHLEADRDYLEI |
| | | | EVGGRRLFFVKAHVRESLLSILLRDWLAMRKQIRSRIPQS |
| | | | TPEEAVLLDKQQAAIKVVCNSVYGFTGVQHGLLPCLHVAA |
| | | | TVTTIGREMLLATRAYVHARWAEFDQLLADFPEAAGMRAP |
| | | | GPYSMRIIYGDTDSIFVLCRGLTAAGLVAMGDKMASHISR |
| | | | ALFLPPIKLECEKTFTKLLLIAKKKYIGVICGGKMLIKGV |
| | | | DLVRKNNCAFINRTSRALVDLLFYDDTVSGAAAALAERPA |
| | | | EEWLARPLPEGLQAFGAVLVDAHRRITDPERDIQDFVLTA |
| | | | ELSRHPRAYTNKRLAHLTVYYKLMARRAQVPSIKDRIPYV |
| | | | IVAQTREVEETVARLAALRELDAAAPGDEPAPPAALPSPA |
| | | | KRPRETPSHADPPGGASKPRKLLVSELAEDPGYAIARGVP |
| | | | LNTDYYFSHLLGAACVTFKALFGNNAKITESLLKRFIPET |
| | | | WHPPDDVAARLRAAGFGPAGAGATAEETRRMLHRAFDTLA |
| 51 | UL30 | MS | MFCAAGGPTSPGGKSAARAASGFFAPHNPRGATQTAPPPC |
| | | | RRQNFYNPHLAQTGTQPKAPGPAQRHTYYSECDEFRFIAP |
| | | | RSLDEDAPAEQRTGVHDGRLRRAPKVYCGGDERDVLRVGP |
| | | | EGFWPRRLRLWGGADHAPKGFDPTVTVFHVYDILEHVEHA |
| | | | YSMRAAQLHERFMDAITPAGTVITLLGLTPEGHRVAVHVY |
| | | | GTRQYFYMNKAEVDRHLQCRAPRDLCERLAAALRESPGAS |
| | | | FRGISADHFEAEVVERADVYYYETRPTLYYRVFVRSGRAL |
| | | | AYLCDNFCPAIRKYEGGVDATTRFILDNPGFVTFGWYRLK |
| | | | PGRGNAPAQPRPPTAFGTSSDVEFNCTADNLAVEGAMCDL |
| | | | PAYKLMCFDIECKAGGEDELAFPVAERPEDLVIQISCLLY |
| | | | DLSTTALEHILLESLGSCDLPESHLSDLASRGLPAPVVLE |
| | | | FDSEFEMLLAFMTFVKQYGPEFVTGYNIINFDWPFVLTKL |
| | | | TEIYKVPLDGYGRMNGRGVFRVWDIGQSHFQKRSKIKVNG |
| | | | MVNIDMYGIITDKVKLSSYKLNAVAEAVLKDKKKDLSYRD |
| | | | IPAYYASGPAQRGVIGEYCVQDSLLVGQLFFKFLPHLELS |
| | | | AVARLAGINITRTIYDGQQIRVFTCLLRLAGQKGFILPDT |
| | | | QGRFRGLDKEAPKRPAVPRGEGERPGDGNGDEDKDDDEDE |
| | | | DGDEREEVARETGGRHVGYQGARVLDPTSGFHVDPVVVFD |
| | | | FASLYPSIIQAHNLCFSTLSLRPEAVAHLEADRDYLEIEV |
| | | | GGRRLFFVKAHVRESLLSILLRDWLAMRKQIRSRIPQSTP |
| | | | EEAVLLDKQQAAIKVVCNSVYGFTGVQHGLLPCLHVAATV |
| | | | TTIGREMLLATRAYVHARWAEFDQLLADFPEAAGMRAPGP |
| | | | YSMRIIYGDTDSIFVLCRGLTAAGLVAMGDKMASHISRAL |
| | | | FLPPIKLECEKTFTKLLLIAKKKYIGVICGGKMLIKGVDL |
| | | | VRKNNCAFINRTSRALVDLLFYDDTVSGAAAALAERPAEE |
| | | | WLARPLPEGLQAFGAVLVDAHRRITDPERDIQDFVLTAEL |
| | | | SRHPRAYTNKRLAHLTVYYKLMARRAQVPSIKDRIPYVIV |
| | | | AQTREVEETVARLAALRELDAAAPGDEPAPPAALPSPAKR |
| | | | PRETPSHADPPGGASKPRKLLVSELAEDPGYAIARGVPLN |
| | | | TDYYFSHLLGAACVTFKALFGNNAKITESLLKRFIPETWH |
| | | | PPDDVAARLRAAGFGPAGAGATAEETRRMLHRAFDTLA |
| 52 | UL52 | HG52 | MGTEDCDHEGRSVAAPVEVTALYATDGCVITSSLALLTNC |
| | | | LLGAEPLYIFSYDAYRSDAPNGPTGAPTEQERFEGSRALY |
| | | | RDAGGLNGDSFRVTFCLLGTEVGVTHHPKGRTRPMFVCRF |
| | | | ERADDVAVLQDALGRGTPLLPAHVTATLDLEATFALHANI |
| | | | IMALTVAIVHNAPARIGSGSTAPLYEPGESMRSVVGRMSL |
| | | | GQRGLTTLFVHHEARVLGAYRRAYYGSAQSPFWFLSKFGP |
| | | | DEKSLVLAARYYLLQAPRLGGAGATYDLQAVKDICATYAI |
| | | | PHDPRPDTLSAASLTSFAAITRFCCTSQYSRGAAAAGFPL |
| | | | YVERRIAADVRETGALEKFIAHDRSCLRVSDREFITYIYL |
| | | | AHFECFSPPRLATHLRAVTTHDPSPAASTEQPSPLGREAV |
| | | | EQFFRHVRAQLNIREYVKQNVTPRETALAGDAAAAYLRAR |
| | | | TYAPAALTPAPAYCGVADSSTKMMGRLAEAERLLVPHGWP |
| | | | AFAPTTPGDDAGGGTAAPQTCGIVKRLLKLAATEQQGTTP |
| | | | PAIAALMQDASVQTPLPVYRITMSPTGQAFAAAARDDWAR |
| | | | VTRDARPPEATVVADAAAAPEPGALGRRLTRRICARGPAL |
| | | | PPGGLAVGGQMYVNRNEIFNAALAVTNIILDLDIALKEPV |
| | | | PFPRLHEALGHFRRGALAAVQLLFPAARVDPDAYPCYFFK |
| | | | SACRPRAPPVCAGDGPSAGGDDGDGDWFPDAGGPGDEEWE |
| | | | EDTDPMDTTHGPLPDDEAAYLDLLHEQIPAATPSEPDSVV |
| | | | CSCADKIGLRVCLPVPAPYVVHGSLTMRGVARVIQQAVLL |
| | | | DRDFVEAVGSHVKNFLLIDTGVYAHGHSLRLPYFAKIGPD |
| | | | GSACGRLLPVFVIPPACEDVPAFVAAHADPRRFHFHAPPM |
| | | | FSAAPREIRVLHSLGGDYVSFFEKKASRNALEHFGRRETL |
| | | | TEVLGRYDVRPDAGETVEGFASELLGRIVACIEAHFPEHA |
| | | | REYQAVSVRRAVIKDDWVLLQLIPGRGALNQSLSCLRFKH |
| | | | GRASRATARTFLALSVGTNNRLCASLCQQCFATKCDNNRL |
| | | | HTLFTVDAGTPCSRSAPSSTSRPSSS |
| 53 | UL52 | 333 | MGTEDCDHEGRSVAAPVEVTALYATDGCVITSSLALLTNC |
| | | | LLGAEPLYIFSYDAYRPDAPNGPTGAPTEQERFEGSRALY |
| | | | RDAGGLNGDSFRVTFCLLGTEVGVTHHPKGRTRPMFVCRF |
| | | | ERADDVAVLQDALGRGTPLLPAHITATLDLEATFALHANI |
| | | | IMALTVAIVHNAPARIGSGSTAPLYEPGESMRSVVGRMSL |
| | | | GQRGLTTLFVHHEARVLAAYRRAYYGSAQSPFWFLSKFGP |
| | | | DEKSLVLAARYYLLQAPRLGGAGATYDLQAVKDICATYAI |
| | | | PHDPRPDTLSAASLTSFAAITRFCCTSQYSRGAAAAGFPL |
| | | | YVERRIAADVRETGALEKFIAHDRSCLRVSDREFITYIYL |
| | | | AHFECFSPPRLATHLRAVTTHDPSPAASTEQPSPLGREAV |
| | | | EQFFRHVRAQLNIREYVKQNVTPRETALAGDAAAAYLRAR |
| | | | TYAPAALTPAPAYCGVADSSTKMMGRLAEAERLLVPHGWP |
| | | | AFAPTTPGDDAGGGTAAPQTCGIVKRLLKLAATEQQGTTP |
| | | | PAIAALMQDASVQTPLPVYRITMSPTGQAFAAAARDDWAR |
| | | | VTRDARPPEATVVADAAAAPEPGALGRRLTRRICARGPAL |
| | | | PPGGLAVGGQMYVNRNEIFNAALAVTNIILDLDIALKEPV |
| | | | PFPRLHEALGHFRRGALAAVQLLFPAARVDPDAYPCYFFK |
| | | | SACRPRAPPVCAGDGPSAGGDDGDGDWFPDAGGDDGDEEW |
| | | | EEDTDPMDTTHGPLPDDEAAYLDLLHEQIPAATPSEPDSV |
| | | | VCSCADKIGLRVCLPVPAPYVVHGSLTMRGVARVIQQAVL |
| | | | LDRDFVEAVGSHVKNFLLIDTGVYAHGHSLRLPYFAKIGP |
| | | | DGSACGRLLPVFVIPPACEDVPAFVAAHADPRRFHFHAPP |
| | | | MFSAAPREIRVLHSLGGDYVSFFEKKASRNALEHFGRRET |
| | | | LTEVLGRYDVRPDAGETVEGFASELLGRIVACIEAHFPEH |
| | | | AREYQAVSVRRAVIKDDWVLLQLIPGRGALNQSLSCLRFK |
| | | | HGRASRATARTFLALSVGTNNRLCASLCQQCFATKCDNNR |
| | | | LHTLFTVDAGTPCSRSAPSSTSRPSSS |
| 54 | UL52 | MS | MGTEDCDHEGRSVAAPVEVTALYATDGCVITSSLALLTNC |
| | | | LLGAEPLYIFSYDAYRPDAPNGPTGAPTEQERFEGSRALY |
| | | | RDAGGLNGDSFRVTFCLLGTEVGVTHHPKGRTRPMFVCRF |
| | | | ERADDVAVLQDALGRGTPLLPAHITATLDLEATFALHANI |
| | | | IMALTVAIVHNAPARIGSGSTAPLYEPGESMRSVVGRMSL |
| | | | GQRGLTTLFVHHEARVLAAYRRAYYGSAQSPFWFLSKFGP |
| | | | DEKSLVLAARYYLLQAPRLGGAGATYDLQAVKDICATYAI |
| | | | PHDPRPDTLSAASLTSFAAITRFCCTSQYSRGAAAAGFPL |
| | | | YVERRIAADVRETSALEKFIAHDRSCLRVSDREFITYIYL |
| | | | AHFECFSPPRLATHLRAVTTHDPSPAASTEQPSPLGREAV |
| | | | EQFFRHVRAQLNIREYVKQNVTPRETALAGDAAAAYLRAR |
| | | | TYAPAALTPAPAYCGVADSSTKMMGRLAEAERLLVPHGWP |
| | | | AFAPTTPGDDAGGGTAAPQTCGIVKRLLKLAATEQQGTTP |
| | | | PAIAALMQDASVQTPLPVYRITMSPTGQAFAAAARDDWAR |
| | | | VTRDARPPEATVVADAAAAPEPGALGRRLTRRICARGPAL |
| | | | PPGGLAVGGQMYVNRNEIFNAALAVTNIILDLDIALKEPV |
| | | | PFPRLHEALGHFRRGALAAVQLLFPAARVDPDAYPCYFFK |
| | | | SACRPRAPPVCAGDGPSAGGDDGDGDWFPDAGGPGDEEWE |
| | | | EDTDPMDTTHGPLPDDEAAYLDLLHEQIPAATPSEPDSVV |
| | | | CSCADKIGLRVCLPVPAPYVVHGSLTMRGVARVIQQAVLL |
| | | | DRDFVEAVGSHVKNFLLIDTGVYAHGHSLRLPYFAKIGPD |
| | | | GSACGRLLPVFVIPPACEDVPAFVAAHADPRRFHFHAPPM |
| | | | FSAAPREIRVLHSLGGDYVSFFEKKASRNALEHFGRRETL |
| | | | TEVLGRYDVRPDAGETVEGFASELLGRIVACIEAHFPEHA |
| | | | REYQAVSVRRAVIKDDWVLLQLIPGRGALNQSLSCLRFKH |
| | | | GRASRATARTFLALSVGTNNRLCASLCQQCFATKCDNNRL |
| | | | HTLFTVDAGTPCSRSAPSSTSRPSSS |
| 55 | UL54 | HG52 | MATDIDMLIDLGLDLSDSELEEDALERDEEGRRDDPESDS |
| | | | SGECSSSDEDMEDPCGDGGAEAIDAAIPKGPPARPEDAGT |
| | | | PEASTPRPAARRGADDPPPATTGVWSRLGTRRSASPREPH |
| | | | GGKVARIQPPSTKAPHPRGGRRGRRRGRGRYGPGGADSTP |
| | | | KPRRRVSRNAHNQGGRHPASARTDGPGATHGEARRGGEQL |
| | | | DVSGGPRPRGTRQAPPPLMALSLTPPHADGRAPVPERKAP |
| | | | SADTIDPAVRAVLRSISERAAVERISESFGRSALVMQDPF |
| | | | GGMPFPAANSPWAPVLATQAGGFDAETRRVSWETLVAHGP |
| | | | SLYRTFAANPRAASTAKAMRDCVLRQENLIEALASADETL |
| | | | AWCKMCIHHNLPLRPQDPIIGTAAAVLENLATRLRPFLQC |
| | | | YLKARGLCGLDDLCSRRRLSDIKDIASFVLVILARLANRV |
| | | | ERGVSEIDYTTVGVGAGETMHFYIPGACMAGLIEILDTHR |
| | | | QECSSRVCELTASHTIAPLYVHGKYFYCNSLF |
| 56 | UL54 | 333 | MATDIDMLIDLGLDLSDSELEEDALERDEEGRRDDPESDS |
| | | | SGECSSSDEDMEDPCGDGGAEAIDAAIPKGPPARPEDAGT |
| | | | PEASTPRPAARRGADDPPPATTGVWSRLGTRRSASPREPH |
| | | | GGKVARIQPPSTKAPHPRGGRRGRRRGRGRYGPGGADSTP |
| | | | NPRRRVSRNAHNQGGRHPASARTDGPGATHGEARRGGEQL |
| | | | DVSGGPRPRGTRQAPPPLMALSLTPPHADGRAPVPERKAP |
| | | | SADTIDPAVRAVLRSISERAAVERISESFGRSALVMQDPF |
| | | | GGMPFPAANSPWAPVLATQAGGFDAETRRVSWETLVAHGP |
| | | | SLYRTFAANPRAASTAKAMRDCVLRQENLIEALASADETL |
| | | | AWCKMCIHHNLPLRPQDPIIGTAAAVLENLATRLRPFLQC |
| | | | YLKARGLCGLDDLCSRRRLSDIKDIASFVLVILARLANRV |
| | | | ERGVSEIDYTTVGVGAGETMHFYIPGACMAGLIEILDTHR |
| | | | QECSSRVCELTASHTIAPLYVHGKYFYCNSLF |
| 57 | UL54 | MS | MATDIDMLIDLGLDLSDSELEEDALERDEEGRRDDPESDS |
| | | | SGECSSSDEDMEDPCGDGGAEAIDAAIPKGPPARPEDAGT |
| | | | PEASTPRPAARRGADDPPPATTGVWSRLGTRRSASPREPH |
| | | | GGKVARIQPPSTKAPHPRGGRRGRRRGRGRYGPGGADSTP |
| | | | NPRRRVSRNAHNQGGRHPASARTDGPGATHGEARRGGEQL |
| | | | DVSGGPRPRGTRQAPPPLMALSLTPPHADGRAPVPERKAP |
| | | | SADTIDPAVRAVLRSISERAAVERISESFGRSALVMQDPF |
| | | | GGMPFPAANSPWAPVLATQAGGFDAETRRVSWETLVAHGP |
| | | | SLYRTFAANPRAASTAKAMRDCVLRQENLIEALASADETL |
| | | | AWCKMCIHHNLPLRPQDPIIGTAAAVLENLATRLRPFLQC |
| | | | YLKARGLCGLDDLCSRRRLSDIKDIASFVLVILARLANRV |
| | | | ERGVSEIDYTTVGVGAGETMHFYIPGACMAGLIEILDTHR |
| | | | QECSSRVCELTASHTIAPLYVHGKYFYCNSLF |
| 58 | US 1 | HG52 | MGFVCLFGLVVMGAWGAWGGSQATEYVLRSVIAKEVGDIL |
| | [HSV-2gL] | | RVPCMRTPADDVSWRYEAPSVIDYARIDGIFLRYHCPGLD |
| | | | TFLWDRHAQRAYLVNPFLFAAGFLEDLSHSVFPADTQETT |
| | | | TRRALYKEIRDALGSRKQAVSHAPVRAGCVNFDYSRTRRC |
| | | | VGRRDLRPANTTSTWEPPVSSDDEASSQSKPLATQPPVLA |
| | | | LSNAPPRRVSPTRGRRRHTRLRRN |
| 59 | US 1 | 333 | MGFVCLFGLVVMGAWGAWGGSQATEYVLRSVIAKEVGDIL |
| | [HSV-2gL] | | RVPCMRTPADDVSWRYEAPSVIDYARIDGIFLRYHCPGLD |
| | | | TFLWDRHAQRAYLVNPFLFAAGFLEDLSHSVFPADTQETT |
| | | | TRRALYKEIRDALGSRKQAVSHAPVRAGCVNFDYSRTRRC |
| | | | VGRRDLRPANTTSTWEPPVSSDDEASSQSKPLATQPPVLA |
| | | | LSNAPPRRVSPTRGRRRHTRLRRN |
| 60 | US 1 | MS | MGFVCLFGLVVMGAWGAWGGSQATEYVLRSVIAKEVGDIL |
| | [HSV-2gL] | | RVPCMRTPADDVSWRYEAPSVIDYARIDGIFLRYHCPGLD |
| | | | TFLWDRHAQRAYLVNPFLFAAGFLEDLSHSVFPADTQETT |
| | | | TRRALYKEIRDALGSRKQAVSHAPVRAGCVNFDYSRTRRC |
| | | | VGRRDLRPANTTSTWEPPVSSDDEASSQSKPLATQPPVLA |
| | | | LSNAPPRRVSPTRGRRRHTRLRRN |
| 61 | US 1 | G | MGFVCLFGLVVMGAWGAWGGSQATEYVLRSVIAKEVGDIL |
| | [HSV-2gL] | | RVPCMRTPADDVSWRYEAPSVIDYARIDGIFLRYHCPGLD |
| | | | TFLWDRHAQRAYLVNPFLFAAGFLEDLSHSVFPADTQETT |
| | | | TRRALYKEIRDALGSRKQAVSHAPVRAGCVNFDYSRTRRC |
| | | | VGRRDLRPANTTSTWEPPVSSDDEASSQSKPLATQPPVLA |
| | | | LSNAPPRRVSPTRGRRRHTRLRRN |
| 62 | US7 | HG52 | GPTVSLVSDSLVDAGAVGPQGFVEEDLRVFGELHFVGAQV |
| | [HSV-2gl] | | PHTNYYDGIIELFHYPLGNHCPRVVHVVTLTACPRRPAVA |
| | | | FTLCRSTHHAHSPAYPTLELGLARQPLLRVRTATRDYAGL |
| | | | YVLRVWVGSATNASRFVLGVALSANGTFVYNGSDYGSCDP |
| | | | AQLPFSAPRLGPSSVYTPGASRPTPPRTTTPPSSPRDPTP |
| | | | APGDTGTPAPASGEIAPPNSTRSASESRHR |
| 63 | US7 | 333 | MPGRSLQGLAILGLWVCATGLVVRGPTVSLVSDSLVDAGA |
| | [HSV-2gl] | | VGPQGFVEEDLRVFGELHFVGAQVPHTNYYDGIIELFHYP |
| | | | LGNHCPRVVHVVTLTACPRRPAVAFTLCRSTHHAHSPAYP |
| | | | TLELGLARQPLLRVRTATRDYAGLYVLRVWVGSATNASLF |
| | | | VLGVALSANGTFVYNGSDYGSCDPAQLPFSAPRLGPSSVY |
| | | | TPGASRPTPPRTTTSPSSPRDPTPAPGDTGTPAPASGERA |
| | | | PPNSTRSASESRHRLTVAQVIQIAIPASIIAFVFLGSCIC |
| | | | FIHRCQRRYRRPRGQIYNPGGVSCAVNEAAMARLGAELRS |
| | | | HPNTPPKPRRRSSSSTTMPSLTSIAEESEPGPVVLLSVSP |
| | | | RPRSGPTAPQEV |
| 64 | US7 | MS | MPGRSLQGLAILGLWVCATGLVVRGPTVSLVSDSLVDAGA |
| | [HSV-2gI] | | VGPQGFVEEDLRVFGELHFVGAQVPHTNYYDGIIELFHYP |
| | | | LGNHCPRVVHVVTLTACPRRPAVAFTLCRSTHHAHSPAYP |
| | | | TLELGLARQPLLRVRTATRDYAGLYVLRVWVGSATNASRF |
| | | | VLGVALSANGTFVYNGSDYGSCDPAQLPFSAPRLGPSSVY |
| | | | TPGASRPTPPRTTTSPSSPRDPTPAPGDTGTPAPASGERA |
| | | | PPNSTRSASESRHRLTVAQVIQIAIPASIIAFVFLGSCIC |
| | | | FIHRCRRRYRRPRGQIYNPGGVSCAVNEAAMARLGAELRS |
| | | | HPNTPPKPRRRSSSSTTMPSLTSIAEESEPGPVVLLSVSP |
| | | | RPRSGPTAPQEV |
| 65 | US7 | G | MPGRSLQGLAILGLWVCATGLVVRGPTVSLVSDSLVDAGA |
| | [HSV-2gI] | | VGPQGFVEEDLRVFGELHFVGAQVPHTNYYDGIIELFHYP |
| | | | LGNHCPRVVHVVTLTACPRRPAVAFTLCRSTHHAHSPAYP |
| | | | TLELGLARQPLLRVRTATRDYAGLYVLRVWVGSATNASRF |
| | | | VLGVALSANGTFVYNGSDYGSCDPAQLPFSAPRLGPSSVY |
| | | | TPGASRPTPPRTTTPPSSPRDPTPAPGDTGTPAPASGESA |
| | | | PPNSTRSASESRHRLTVAQVIQIAIPASIIAFVFLGSCIC |
| | | | FIHRCQRRYRRPRGQIYNPGGVSCAVNEAAMARLGAELRS |
| | | | HPNTPPKPRRRSSSSTTMPSLTSIAEESEPGPVVLLSVSP |
| | | | RPRSGPTAPQEV |
| 66 | US8 | HG52 | MARGAGLVFFVGVWVVSCLAAAPRTSWKRVTSGEDVVLLP |
| | [HSV-2gE] | | APAERTRAHKLLWAAEPLDACGPLRPSWVALWPPRRVLET |
| | | | VVDAACMRAPEPLAIAYSPPFPAGDEGLYSELAWRDRVAV |
| | | | VNESLVIYGALETDSGLYTLSVVGLSDEARQVASVVLVVE |
| | | | PAPVPTPTPDDYDEEDDAGVSERTPVSVPPPTPPRRPPVA |
| | | | PPTHPRVIPEVSHVRGVTVHMETPEAILFAPGETFGTNVS |
| | | | IHAIAHDDGPYAMDVVWMRFDVPSSCAEMRIYEACLYHPQ |
| | | | LPECLSPADAPCAVSSWAYRLAVRSYAGCSRTTPPPRCFA |
| | | | EARMEPVPGLAWLASTVNLEFQHASPQHAGLYLCVVYVDD |
| | | | HIHAWGHMTISTAAQYRNAVVEQHLPQRQPEPVEPTRPHV |
| | | | RAPHPAPSARGPLRLGAVLGAALLLAALGLSAWACMTCWR |
| | | | RRSWRAVKSRASATGPTYIRVADSELYADWSSDSEGERDG |
| | | | SLWQDPPERPDSPSTNGSGFEILSPTAPSVYPHSEGRKSR |
| | | | RPLTTFGSGSPGRRHSQASYPSVLW |
| 67 | US8 | 333 | RTSWKRVTSGEDVVLLPAPAGPEERTRAHKLLWAAEPLDA |
| | [HSV-2gE | | CGPLRPSWVALWPPRRVLETVVDAACMRAPEPLAIAYSPP |
| | | | FPAGDEGLYSELAWRDRVAVVNESLVIYGALETDSGLYTL |
| | | | SVVGLSDEARQVASVVLVVEPAPVPTPTPDDYDEEDDAGV |
| | | | SERTPVSVPPPTPPRRPPVAPPTHPRVIPEVSHVRGVTVH |
| | | | METPEAILFAPGETFGTNVSIHAIAHDDGPYAMDVVWMRF |
| | | | DVPSSCAEMRIYEACLYHPQLPECLSPADAPCAVSSWAYR |
| | | | LAVRSYAGCSRTTPPPRCFAEARMEPVPGLAWLASTVNLE |
| | | | FQHASPQHAGLYLCVVYVDDHIHAWGHMTISTAAQYRNAV |
| | | | VEQHLPQRQPEPVEPTRPHVRA |
| 68 | US8 | MS | MARGAGLVFFVGVWVVSCLAAAPRTSWKRVTSGEDVVLLP |
| | [HSV-2gE] | | APAERTRAHKLLWAAEPLDACGPLRPSWVALWPPRRVLET |
| | | | VVDAACMRAPEPLAIAYSPPFPAGDEGLYSELAWRDRVAV |
| | | | VNESLVIYGALETDSGLYTLSVVGLSDEARQVASVVLVVE |
| | | | PAPVPTPTPDDYDEEDDAGVSERTPVSVPPPTPPRRPPVA |
| | | | PPTHPRVIPEVSHVRGVTVHMETPEAILFAPGETFGTNVS |
| | | | IHAIAHDDGPYAMDVVWMRFDVPSSCAEMRIYEACLYHPQ |
| | | | LPECLSPADAPCAVSSWAYRLAVRSYAGCSRTTPPPRCFA |
| | | | EARMEPVPGLAWLASTVNLEFQHASPQHAGLYLCVVYVDD |
| | | | HIHAWGHMTISTAAQYRNAVVEQHLPQRQPEPVEPTRPHV |
| | | | RAPHPAPSARGPLRLGAVLGAALLLAALGLSAWACMTCWR |
| | | | RRSWRAVKSRASATGPTYIRVADSELYADWSSDSEGERDG |
| | | | SLWQDPPERPDSPSTNGSGFEILSPTAPSVYPHSEGRKSR |
| | | | RPLTTFGSGSPGRRHSQASYSSVLW |
| 69 | US8 | G | MARGAGLVFFVGVWVVSCLAAAPRTSWKRVTSGEDVVLLP |
| | [HSV-2gE] | | APAGPEERTRAHKLLWAAEPLDACGPLRPSWVALWPPRRV |
| | | | LETVVDAACMRAPEPLAIAYSPPFPAGDEGLYSELAWRDR |
| | | | VAVVNESLVIYGALETDSGLYTLSVVGLSDEARQVASVVL |
| | | | VVEPAPVPTPTPDDYDEEDDAGVSERTPVSVPPPTPPRRP |
| | | | PVAPPTHPRVIPEVSHVRGVTVHMETPEAILFAPGETFGT |
| | | | NVSIHAIAHDDGPYAMDVVWMRFDVPSSCAEMRIYEACLY |
| | | | HPQLPECLSPADAPCAVSSWAYRLAVRSYAGCSRTTPPPR |
| | | | CFAEARMEPVPGLAWLASTVNLEFQHASPQHAGLYLCVVY |
| | | | VDDHIHAWGHMTISTAAQYRNAVVEQHLPQRQPEPVEPTR |
| | | | PHMRAPHPAPSARGPLRLGAVLGAALLLAALGLSAWACMT |
| | | | CWRRRSWRAVKSRASATGPTYIRVADSELYADWSSDSEGE |
| | | | RDGSLWQDPPERPDSPSTNGSGFEILSPTAPSVYPHSEGR |
| | | | KSRRPLTTFGSGSPGRRHSQASYSSVLW |
| 70 | UL22 | HG52 | MGPGLWVVMGVLVGVAGGHDTYWTEQIDPWFLHGLGLART |
| | [HSV-2gH] | | YWRDTNTGRLWLPNTPDASDPQRGRLAPPGELNLTTASVP |
| | | | MLRWYAERFCFVLVTTAEFPRDPGQLLYIPKTYLLGRPRN |
| | | | ASLPELPEAGPTSRPPAEVTQLKGLSHNPGASALLRSRAW |
| | | | VTFAAAPDREGLTFPRGDDGATERHPDGRRNAPPPGPPAG |
| | | | TPRHPTTNLSIAHLHNASVTWLAARGLLRTPGRYVYLSPS |
| | | | ASTWPVGVWTTGGLAFGCDAALVRARYGKGFMGLVISMRD |
| | | | SPPAEIIVVPADKTLARVGNPTDENAPAVLPGPPAGPRYR |
| | | | VFVLGAPTPADNGSALDALRRVAGYPEESTNYAQYMSRAY |
| | | | AEFLGEDPGSGTDARPSLFWRLAGLLASSGFAFVNAAHAH |
| | | | DAIRLSDLLGFLAHSRVLAGLAARGAAGCAADSVFLNVSV |
| | | | LDPAARLRLEARLGHLVAAILEREQSLAAHALGYQLAFVL |
| | | | DSPAAYGAVAPSAARLIDALYAEFLGGRALTAPMVRRALF |
| | | | YATAVLRAPFLAGAPSAEQRERARRGLLITTALCTSDVAA |
| | | | ATHADLRAALARTDHQKNLFWLPDHFSPCAASLRFDLAEG |
| | | | GFILDALAMATRSDIPADVMAQQTRGVASVLTRWAHYNAL |
| | | | IRAFVPEATHQCSGPSHNAEPRILVPITHNASYVVTHTPL |
| | | | PRGIGYKLTGVDVRRPLFITYLTATCEGHAREIEPKRLVR |
| | | | TENRRDLGLVGAVFLRYTPAGEVMSVLLVDTDATQQQLAQ |
| | | | GPVAGTPNVFSSDVPSVALLLFPNGTVIHLLAFDTLPIAT |
| | | | IAPGFLAASALGVVMITAALAGILRVVRTCVPFLWRRE |
| 71 | UL22 | 333 | MGPGLWVVMGVLVGVAGGHDTYWTEQIDPWFLHGLGLART |
| | [HSV-2gH] | | YWRDTNTGRLWLPNTPDASDPQRGRLAPPGELNLTTASVP |
| | | | MLRWYAERFCFVLVTTAEFPRDPGQLLYIPKTYLLGRPRN |
| | | | ASLPELPEAGPTSRPPAEVTQLKGLSHNPGASALLRSRAW |
| | | | VTFAAAPDREGLTFPRGDDGATERHPDGRRNAPPPGPPAG |
| | | | TPRHPTTNLSIAHLHNASVTWLAARGLLRTPGRYVYLSPS |
| | | | ASTWPVGVWTTGGLAFGCDAALVRARYGKGFMGLVISMRD |
| | | | SPPAEIIVVPADKTLARVGNPTDENAPAVLPGPPAGPRYR |
| | | | VFVLGAPTPADNGSALDALRRVAGYPEESTNYAQYMSRAY |
| | | | AEFLGEDPGSGTDARPSLFWRLAGLLASSGFAFVNAAHAH |
| | | | DAIRLSDLLGFLAHSRVLAGLAARGAAGCAADSVFLNVSV |
| | | | LDPAARLRLEARLGHLVAAILEREQSLAAHALGYQLAFVL |
| | | | DSPAAYGAVAPSAARLIDALYAEFLGGRALTAPMVRRALF |
| | | | YATAVLRAPFLAGAPSAEQRERARRGLLITTALCTSDVAA |
| | | | ATHADLRAALARTDHQKNLFWLPDHFSPCAASLRFDLAEG |
| | | | GFILDALAMATRSDIPADVMAQQTRGVASVLTRWAHYNAL |
| | | | IRAFVPEATHQCSGPSHNAEPRILVPITHNASYVVTHTPL |
| | | | PRGIGYKLTGVDVRRPLFITYLTATCEGHAREIEPKRLVR |
| | | | TENRRDLGLVGAVFLRYTPAGEVMSVLLVDTDATQQQLAQ |
| | | | GPVAGTPNVFSSDVPSVALLLFPNGTVIHLLAFDTLPIAT |
| | | | IAPGFLAASALGVVMITAALAGILRVVRTCVPFLWRRE |
| 72 | UL22 | MS | MGPGLWVVMGVLVGVAGGHDTYWTEQIDPWFLHGLGLART |
| | [HSV-2gH] | | YWRDTNTGRLWLPNTPDASDPQRGRLAPPGELNLTTASVP |
| | | | MLRWYAERFCFVLVTTAEFPRDPGQLLYIPKTYLLGRPRN |
| | | | ASLPELPEAGPTSRPPAEVTQLKGLSHNPGASALLRSRAW |
| | | | VTFAAAPDREGLTFPRGDDGATERHPDGRRNAPPPGPPAG |
| | | | APRHPTTNLSIAHLHNASVTWLAARGLLRTPGRYVYLSPS |
| | | | ASTWPVGVWTTGGLAFGCDAALVRARYGKGFMGLVISMRD |
| | | | SPPAEIIVVPADKTLARVGNPTDENAPAVLPGPPAGPRYR |
| | | | VFVLGAPTPADNGSALDALRRVAGYPEESTNYAQYMSRAY |
| | | | AEFLGEDPGSGTDARPSLFWRLAGLLASSGFAFVNAAHAH |
| | | | DAIRLSDLLGFLTHSRVLAGLAARGAAGCAADSVFLNVSV |
| | | | LDPAARLRLEARLGHLVAAILEREQSLAAHALGYQLAFVL |
| | | | DSPAAYGAVAPSAARLIDALYAEFLGGRALTAPMVRRALF |
| | | | YATAVLRAPFLAGAPSAEQRERARRGLLITTALCTSDVAA |
| | | | ATHADLRAALARTDHQKNLFWLPDHFSPCAASLRFDLAEG |
| | | | GFILDALAMATRSDIPADVMAQQTRGVASALTRWAHYNAL |
| | | | IRAFVPEATHQCSGPSHNAEPRILVPITHNASYVVTHTPL |
| | | | PRGIGYKLTGVDVRRPLFITYLTATCEGHAREIEPKRLVR |
| | | | TENRRDLGLVGAVFLRYTPAGEVMSVLLVDTDATQQQLAQ |
| | | | GPVAGTPNVESSDVPSVALLLFPNGTVIHLLAFDTLPIAT |
| | | | IAPGFLAASALGVVMITAALAGILRVVRTCVPFLWRRE |
| 73 | UL22 | G | MGPGLWVVMGVLVGVAGGHDTYWTEQIDPWFLHGLGLART |
| | [HSV-2gH] | | YWRDTNTGRLWLPNTPDASDPQRGRLAPPGELNLTTASVP |
| | | | MLRWYAERFCFVLVTTAEFPRDPGQLLYIPKTYLLGRPRN |
| | | | ASLPELPEAGPTSRPPAEVTQLKGLSHNPGASALLRSRAW |
| | | | VTFAAAPDREGLTFPRGDDGATERHPDGRRNAPPPGPPAG |
| | | | TPRHPTTNLSIAHLHNASVTWLAARGLLRTPGRYVYLSPS |
| | | | ASTWPVGVWTTGGLAFGCDAALVRARYGKGFMGLVISMRD |
| | | | SPPAEIIVVPADKTLARVGNPTDENAPAVLPGPPAGPRYR |
| | | | VFVLGAPTPADNGSALDALRRVAGYPEESTNYAQYMSRAY |
| | | | AEFLGEDPGSGTDARPSLFWRLAGLLASSGFAFVNAAHAH |
| | | | DAIRLSDLLGFLAHSRVLAGLAARGAAGCAADSVFLNVSV |
| | | | LDPAARLRLEARLGHLVAAILEREQSLAAHALGYQLAFVL |
| | | | DSPAAYGAVAPSAARLIDALYAEFLGGRALTAPMVRRALF |
| | | | YATAVLRAPFLAGAPSAE1QRERARRGLLITTALCTSDVA |
| | | | AATHADLRAALARTDHQKNLFWLPDHFSPCAASLRFDLAE |
| | | | GGFILDALAMATRSDIPADVMAQQTRGVASVLTRWAHYNA |
| | | | LIRAFVPEATHQCSGPSHNAEPRILVPITHNASYVVTHTP |
| | | | LPRGIGYKLTGVDVRRPLFITYLTATCEGHAREIEPKRLV |
| | | | RTENRRDLGLVGAVFLRYTPAGEVMSVLLVDTDATQQQLA |
| | | | QGPVAGTPNVFSSDVPSVALLLFPNGTVIHLLAFDTLPIA |
| | | | TIAPGFLAASALGVVMITAALAGILRVVRTCVPFLWRRE |
| 221 | US6 | | MGRLTSGVGTAALLVVAVGLRVVCAKYALADPSLKMADPN |
| | [HSV-2gD] | | RFRGKNLPVLDQLTDPPGVKRVYHIQPSLEDPFQPPSIPI |
| | | | TVYYAVLERACRSVLLHAPSEAPQIVRGASDEARKHTYNL |
| | | | TIAWYRMGDNCAIPITVMEYTECPYNKSLGVCPIRTQPRW |
| | | | SYYDSFSAVSEDNLGFLMHAPAFETAGTYLRLVKINDWTE |
| | | | ITQFILEHRARASCKYALPLRIPPAACLTSKAYQQGVTVD |
| | | | SIGMLPRFIPENQRTVALYSLKIAGWHGPKPPYTSTLLPP |
| | | | ELSDTTNATQPELVPEDPEDSALLEDPAGTVSSQIPPNWH |
| | | | IPSIQDVAPHH |
| 222 | UL44 | 333 | SPGRTITVGPRGNASNAAPSASPRNASAPRTTPTPPQPRK |
| | [HSV-2gC] | | ATKSKASTAKPAPPPKTGPPKTSSEPVRCNRHDPLARYGS |
| | | | RVQIRCRFPNSTRTESRLQIWRYATATDAEIGTAPSLEEV |
| | | | MVNVSAPPGGQLVYDSAPNRTDPHVIWAEGAGPGASPRLY |
| | | | SVVGPLGRQRLIIEELTLETQGMYYWVWGRTDRPSAYGTW |
| | | | VRVRVFRLPSLTIHPHAVLEGQPFKATCTAATYYPGNRAE |
| | | | FVWFEDGRRVFDPAQIHTQTQENPDGFSTVSTVTSAAVGG |
| | | | QGPPRTFTCQLTWHRDSVSFSRRNASGTASVLPRPTITME |
| | | | FTGDHAVCTAGCVPEGVTFAWFLGDDSSPAEKVAVASQTS |
| | | | CGRPGTATIRSTLPVSYEQTEYICRLAGYPDGIPVLEHHG |
| | | | SHQPPPRDPTERQVIRAVEGAGIGVAVLVAVVLAGTAVVY |
| | | | LTHASSVRYRRLR |

In some embodiments, the present disclosure provides certain HSV antigen constructs (e.g., HSV-1 antigen constructs, HSV-2 antigen constructs, or a combination thereof) particularly useful in effective vaccination.

In various embodiments, an HSV antigen construct includes and/or encodes a plurality of HSV antigens (e.g., a plurality of HSV antigens that are or include one or more T cell and/or B cell antigens for HSV). As disclosed herein, T cell antigens include, e.g., CD4 T cell antigens and/or CD8 T cells. In some embodiments, an HSV antigen is a T cell antigen. In some embodiments, an HSV antigen is a B cell antigen.

In certain embodiments, an HSV antigen construct can include and/or encode at least one of UL1, UL21, UL27, UL29, UL39, UL40, UL46, UL47, UL48, UL49, RS1, RL2, UL5, UL9, UL19, UL25, UL30, UL52, US1, US7, US8, UL22, and/or UL54 or fragments thereof. In certain embodiments, an HSV antigen construct can include and/or encode at least one of UL1, UL21, UL27, UL29, UL39, UL40, UL46, UL47, UL48, and/or UL49 or fragments thereof. In certain embodiments, an HSV antigen construct can include and/or encode at least one of RS1, RL2, UL5, UL9, UL19, UL25, UL30, UL52, US1, US7, US8, UL22, and/or UL54 or fragments thereof.

In certain embodiments, an HSV antigen construct can include and/or encode a plurality of (e.g., 1, 2, 3, 4, 5, 6, 7, 8, 9, 10, 11, 12, 13, 14, 15, 16, 17, 18, or 19 of) UL1, UL21, UL27, UL29, UL39, UL40, UL46, UL47, UL48, UL49, RS1, RL2, UL5, UL9, UL19, UL25, UL30, UL52, US1, US7, US8, UL22, and/or UL54 or fragments thereof. In certain embodiments, an HSV antigen construct can include and/or encode a plurality of (e.g., 1, 2, 3, 4, 5, 6, 7, 8, 9, or 10 of) UL1, UL21, UL27, UL29, UL39, UL40, UL46, UL47, UL48, and/or UL49 or fragments thereof. In certain embodiments, an HSV antigen construct can include and/or encode a plurality of (e.g., 1, 2, 3, 4, 5, 6, 7, 8, or 9 of) RS1, RL2, UL5, UL9, UL19, UL25, UL30, UL52, US1, US7, US8, UL22, and/or UL54 or fragments thereof.

In some embodiments, an HSV antigen (e.g., a T cell or B cell antigen for HSV) is or includes a UL1 polypeptide or fragment thereof. In various embodiments, a UL1 polypeptide or fragment thereof has at least 80% sequence identity with a UL1 amino acid sequence set forth in Table 3 or otherwise known in the art, or a corresponding fragment thereof (e.g., at least 80%, 85%, 90%, 91%, 92%, 93%, 94%, 95%, 96%, 97%, 99%, or 100% sequence identity). Examples of UL1 polypeptides known in the art include UL1 polypeptides encoded by known HSV strains such as, without limitation, HG52, G, 333, and MS strains. In some embodiments, a UL1 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 1, 2 and/or 3.

In some embodiments, an HSV antigen (e.g., a T cell or B cell antigen for HSV) is or includes a UL21 polypeptide or fragment thereof. In various embodiments, a UL21 polypeptide or fragment thereof has at least 80% sequence identity with a UL21 amino acid sequence set forth in Table 3 or otherwise known in the art, or a corresponding fragment thereof (e.g., at least 80%, 85%, 90%, 91%, 92%, 93%, 94%, 95%, 96%, 97%, 99%, or 100% sequence identity). Examples of UL21 polypeptides known in the art include UL21 polypeptides encoded by known HSV strains such as, without limitation, HG52, G, 333, and MS strains. In some embodiments, a UL21 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 4, 5 and/or 6.

The UL27 open reading frame encodes HSV gB (also referred to herein as UL27 polypeptide). In some embodiments, an HSV antigen (e.g., a T cell or B cell antigen for HSV) is or includes a UL27 polypeptide or fragment thereof. In various embodiments, a UL27 polypeptide or fragment thereof has at least 80% sequence identity with a UL27 amino acid sequence set forth in Table 3 or otherwise known in the art, or a corresponding fragment thereof (e.g., at least 80%, 85%, 90%, 91%, 92%, 93%, 94%, 95%, 96%, 97%, 99%, or 100% sequence identity). Examples of UL27 polypeptides known in the art include UL27 polypeptides encoded by known HSV strains such as, without limitation, HG52, G, 333, and MS strains. In some embodiments, a UL27 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 7, 8, 9 and/or 74.

In some embodiments, an HSV antigen (e.g., a T cell or B cell antigen for HSV) is or includes a UL29 polypeptide or fragment thereof. In various embodiments, a UL29 polypeptide or fragment thereof has at least 80% sequence identity with a UL29 amino acid sequence set forth in Table 3 or otherwise known in the art, or a corresponding fragment thereof (e.g., at least 80%, 85%, 90%, 91%, 92%, 93%, 94%, 95%, 96%, 97%, 99%, or 100% sequence identity). Examples of UL29 polypeptides known in the art include UL29 polypeptides encoded by known HSV strains such as, without limitation, HG52, G, 333, and MS strains. In some embodiments, a UL29 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 10, 11, and/or 12.

In some embodiments, an HSV antigen (e.g., a T cell or B cell antigen for HSV) is or includes a UL39 polypeptide or fragment thereof. In various embodiments, a UL39 polypeptide or fragment thereof has at least 80% sequence identity with a UL39 amino acid sequence set forth in Table 3 or otherwise known in the art, or a corresponding fragment thereof (e.g., at least 80%, 85%, 90%, 91%, 92%, 93%, 94%, 95%, 96%, 97%, 99%, or 100% sequence identity). Examples of UL39 polypeptides known in the art include UL39 polypeptides encoded by known HSV strains such as, without limitation, HG52, G, 333, and MS strains. In some embodiments, a UL39 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 13, 14 and/or 15.

In some embodiments, an HSV antigen (e.g., a T cell or B cell antigen for HSV) is or includes a UL40 polypeptide or fragment thereof. In various embodiments, a UL40 polypeptide or fragment thereof has at least 80% sequence identity with a UL40 amino acid sequence set forth in Table 3 or otherwise known in the art, or a corresponding fragment thereof (e.g., at least 80%, 85%, 90%, 91%, 92%, 93%, 94%, 95%, 96%, 97%, 99%, or 100% sequence identity). Examples of UL40 polypeptides known in the art include UL40 polypeptides encoded by known HSV strains such as, without limitation, HG52, G, 333, and MS strains. In some embodiments, a UL40 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 16, 17 and/or 18.

In some embodiments, an HSV antigen (e.g., a T cell or B cell antigen for HSV) is or includes a UL46 polypeptide or fragment thereof. In various embodiments, a UL46 polypeptide or fragment thereof has at least 80% sequence identity with a UL46 amino acid sequence set forth in Table 3 or otherwise known in the art, or a corresponding fragment thereof (e.g., at least 80%, 85%, 90%, 91%, 92%, 93%, 94%, 95%, 96%, 97%, 99%, or 100% sequence identity). Examples of UL46 polypeptides known in the art include UL46 polypeptides encoded by known HSV strains such as, without limitation, HG52, G, 333, and MS strains. In some embodiments, a UL46 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 19, 20 and/or 21.

In some embodiments, an HSV antigen (e.g., a T cell or B cell antigen for HSV) is or includes a UL47 polypeptide or fragment thereof. In various embodiments, a UL47 polypeptide or fragment thereof has at least 80% sequence identity with a UL47 amino acid sequence set forth in Table 3 or otherwise known in the art, or a corresponding fragment thereof (e.g., at least 80%, 85%, 90%, 91%, 92%, 93%, 94%, 95%, 96%, 97%, 99%, or 100% sequence identity). Examples of UL47 polypeptides known in the art include UL47 polypeptides encoded by known HSV strains such as, without limitation, HG52, G, 333, and MS strains. In some embodiments, a UL47 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 22, 23 and/or 24.

In some embodiments, an HSV antigen (e.g., a T cell or B cell antigen for HSV) is or includes a UL48 polypeptide or fragment thereof. In various embodiments, a UL48 polypeptide or fragment thereof has at least 80% sequence identity with a UL48 amino acid sequence set forth in Table 3 or otherwise known in the art, or a corresponding fragment thereof (e.g., at least 80%, 85%, 90%, 91%, 92%, 93%, 94%, 95%, 96%, 97%, 99%, or 100% sequence identity). Examples of UL48 polypeptides known in the art include UL48 polypeptides encoded by known HSV strains such as, without limitation, HG52, G, 333, and MS strains. In some embodiments, a UL48 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 25, 26 and/or 27.

In some embodiments, an HSV antigen (e.g., a T cell or B cell antigen for HSV) is or includes a UL49 polypeptide or fragment thereof. In various embodiments, a UL49 polypeptide or fragment thereof has at least 80% sequence identity with a UL49 amino acid sequence set forth in Table 3 or otherwise known in the art, or a corresponding fragment thereof (e.g., at least 80%, 85%, 90%, 91%, 92%, 93%, 94%, 95%, 96%, 97%, 99%, or 100% sequence identity). Examples of UL49 polypeptides known in the art include UL49 polypeptides encoded by known HSV strains such as, without limitation, HG52, G, 333, and MS strains. In some embodiments, a UL49 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 28, 29 and/or 30.

In some embodiments, an HSV antigen (e.g., a T cell or B cell antigen for HSV) is or includes a RS1 polypeptide or fragment thereof. In various embodiments, a RS1 polypeptide or fragment thereof has at least 80% sequence identity with a RS1 amino acid sequence set forth in Table 3 or otherwise known in the art, or a corresponding fragment thereof (e.g., at least 80%, 85%, 90%, 91%, 92%, 93%, 94%, 95%, 96%, 97%, 99%, or 100% sequence identity). Examples of RS1 polypeptides known in the art include RS1 polypeptides encoded by known HSV strains such as, without limitation, HG52, G, 333, and MS strains. In some embodiments, a RS1 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 31, 32 and/or 33.

In some embodiments, an HSV antigen (e.g., a T cell or B cell antigen for HSV) is or includes a RL2 polypeptide or fragment thereof. In various embodiments, a RL2 polypeptide or fragment thereof has at least 80% sequence identity with a RL2 amino acid sequence set forth in Table 3 or otherwise known in the art, or a corresponding fragment thereof (e.g., at least 80%, 85%, 90%, 91%, 92%, 93%, 94%, 95%, 96%, 97%, 99%, or 100% sequence identity). Examples of RL2 polypeptides known in the art include RL2 polypeptides encoded by known HSV strains such as, without limitation, HG52, G, 333, and MS strains. In some embodiments, a RL2 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 34, 35 and/or 36.

In some embodiments, an HSV antigen (e.g., a T cell or B cell antigen for HSV) is or includes a UL5 polypeptide or fragment thereof. In various embodiments, a UL5 polypeptide or fragment thereof has at least 80% sequence identity with a UL5 amino acid sequence set forth in Table 3 or otherwise known in the art, or a corresponding fragment thereof (e.g., at least 80%, 85%, 90%, 91%, 92%, 93%, 94%, 95%, 96%, 97%, 99%, or 100% sequence identity). Examples of UL5 polypeptides known in the art include UL5 polypeptides encoded by known HSV strains such as, without limitation, HG52, G, 333, and MS strains. In some embodiments, a UL5 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 37, 38 and/or 39.

In some embodiments, an HSV antigen (e.g., a T cell or B cell antigen for HSV) is or includes a UL9 polypeptide or fragment thereof. In various embodiments, a UL9 polypeptide or fragment thereof has at least 80% sequence identity with a UL9 amino acid sequence set forth in Table 3 or otherwise known in the art, or a corresponding fragment thereof (e.g., at least 80%, 85%, 90%, 91%, 92%, 93%, 94%, 95%, 96%, 97%, 99%, or 100% sequence identity). Examples of UL9 polypeptides known in the art include UL9 polypeptides encoded by known HSV strains such as, without limitation, HG52, G, 333, and MS strains. In some embodiments, a UL9 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 40, 41 and/or 42.

In some embodiments, an HSV antigen (e.g., a T cell or B cell antigen for HSV) is or includes a UL19 polypeptide or fragment thereof. In various embodiments, a UL19 polypeptide or fragment thereof has at least 80% sequence identity with a UL19 amino acid sequence set forth in Table 3 or otherwise known in the art, or a corresponding fragment thereof (e.g., at least 80%, 85%, 90%, 91%, 92%, 93%, 94%, 95%, 96%, 97%, 99%, or 100% sequence identity). Examples of UL19 polypeptides known in the art include UL19 polypeptides encoded by known HSV strains such as, without limitation, HG52, G, 333, and MS strains. In some embodiments, a UL19 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 43, 44 and/or 45.

In some embodiments, an HSV antigen (e.g., a T cell or B cell antigen for HSV) is or includes a UL25 polypeptide or fragment thereof. In various embodiments, a UL25 polypeptide or fragment thereof has at least 80% sequence identity with a UL25 amino acid sequence set forth in Table 3 or otherwise known in the art, or a corresponding fragment thereof (e.g., at least 80%, 85%, 90%, 91%, 92%, 93%, 94%, 95%, 96%, 97%, 99%, or 100% sequence identity). Examples of UL25 polypeptides known in the art include UL25 polypeptides encoded by known HSV strains such as, without limitation, HG52, G, 333, and MS strains. In some embodiments, a UL25 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 46, 47 and/or 48.

In some embodiments, an HSV antigen (e.g., a T cell or B cell antigen for HSV) is or includes a UL30 polypeptide or fragment thereof. In various embodiments, a UL30 polypeptide or fragment thereof has at least 80% sequence identity with a UL30 amino acid sequence set forth in Table 3 or otherwise known in the art, or a corresponding fragment thereof (e.g., at least 80%, 85%, 90%, 91%, 92%, 93%, 94%, 95%, 96%, 97%, 99%, or 100% sequence identity). Examples of UL30 polypeptides known in the art include UL30 polypeptides encoded by known HSV strains such as, without limitation, HG52, G, 333, and MS strains. In some embodiments, a UL30 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 49, 50 and/or 51.

In some embodiments, an HSV antigen (e.g., a T cell or B cell antigen for HSV) is or includes a UL52 polypeptide or fragment thereof. In various embodiments, a UL52 polypeptide or fragment thereof has at least 80% sequence identity with a UL52 amino acid sequence set forth in Table 3 or otherwise known in the art, or a corresponding fragment thereof (e.g., at least 80%, 85%, 90%, 91%, 92%, 93%, 94%, 95%, 96%, 97%, 99%, or 100% sequence identity). Examples of UL52 polypeptides known in the art include UL52 polypeptides encoded by known HSV strains such as, without limitation, HG52, G, 333, and MS strains. In some embodiments, a UL52 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 52, 53 and/or 54.

The US1 open reading frame encodes HSV gL (also referred to herein as US1 polypeptide). In some embodiments, an HSV antigen (e.g., a T cell or B cell antigen for HSV) is or includes a US1 polypeptide or fragment thereof. In various embodiments, a US1 polypeptide or fragment thereof has at least 80% sequence identity with a US1 amino acid sequence set forth in Table 3 or otherwise known in the art, or a corresponding fragment thereof (e.g., at least 80%, 85%, 90%, 91%, 92%, 93%, 94%, 95%, 96%, 97%, 99%, or 100% sequence identity). Examples of US1 polypeptides known in the art include US1 polypeptides encoded by known HSV strains such as, without limitation, HG52, G, 333, and MS strains. In some embodiments, a US1 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 58, 59, 60 and/or 61.

The US7 open reading frame encodes HSV gI (also referred to herein as US7 polypeptide). In some embodiments, an HSV antigen (e.g., a T cell or B cell antigen for HSV) is or includes a US7 polypeptide or fragment thereof. In various embodiments, a US7 polypeptide or fragment thereof has at least 80% sequence identity with a US7 amino acid sequence set forth in Table 3 or otherwise known in the art, or a corresponding fragment thereof (e.g., at least 80%, 85%, 90%, 91%, 92%, 93%, 94%, 95%, 96%, 97%, 99%, or 100% sequence identity). Examples of US7 polypeptides known in the art include US7 polypeptides encoded by known HSV strains such as, without limitation, HG52, G, 333, and MS strains. In some embodiments, a US7 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 62, 63, 64 and/or 65.

The US8 open reading frame encodes HSV gE (also referred to herein as US8 polypeptide). In some embodiments, an HSV antigen is (e.g., a T cell or B cell antigen for HSV) or includes a US8 polypeptide or fragment thereof. In various embodiments, a US8 polypeptide or fragment thereof has at least 80% sequence identity with a US8 amino acid sequence set forth in Table 3 or otherwise known in the art, or a corresponding fragment thereof (e.g., at least 80%, 85%, 90%, 91%, 92%, 93%, 94%, 95%, 96%, 97%, 99%, or 100% sequence identity). Examples of US8 polypeptides known in the art include US8 polypeptides encoded by known HSV strains such as, without limitation, HG52, G, 333, and MS strains. In some embodiments, a US8 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 66, 67, 68 and/or 69.

The UL22 open reading frame encodes HSV gH (also referred to herein as UL22 polypeptide). In some embodiments, an HSV antigen is (e.g., a T cell or B cell antigen for HSV) or includes a UL22 polypeptide or fragment thereof. In various embodiments, a UL22 polypeptide or fragment thereof has at least 80% sequence identity with a UL22 amino acid sequence set forth in Table 3 or otherwise known in the art, or a corresponding fragment thereof (e.g., at least 80%, 85%, 90%, 91%, 92%, 93%, 94%, 95%, 96%, 97%, 99%, or 100% sequence identity). Examples of UL22 polypeptides known in the art include UL22 polypeptides encoded by known HSV strains such as, without limitation, HG52, G, 333, and MS strains. In some embodiments, a UL22 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 70, 71, 72 and/or 73.

In some embodiments, an HSV antigen (e.g., a T cell or B cell antigen for HSV) is or includes a UL54 polypeptide or fragment thereof. In various embodiments, a UL54 polypeptide or fragment thereof has at least 80% sequence identity with a UL54 amino acid sequence set forth in Table 3 or otherwise known in the art, or a corresponding fragment thereof (e.g., at least 80%, 85%, 90%, 91%, 92%, 93%, 94%, 95%, 96%, 97%, 99%, or 100% sequence identity). Examples of UL54 polypeptides known in the art include UL54 polypeptides encoded by known HSV strains such as, without limitation, HG52, G, 333, and MS strains. In some embodiments, a UL54 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 55, 56, and/or 57.

In certain embodiments, an HSV antigen construct can include and/or encode one or more HSV antigens including one or more T cell antigens (e.g., CD4 and/or CD8 T cell antigens) for HSV of the present disclosure and one or more HSV antigens that is not a T cell antigen of the present disclosure. In certain embodiments, an HSV antigen construct can include and/or encode one or more HSV antigens including one or more B cell antigens for HSV of the present disclosure and one or more HSV antigens that is not a B cell antigen of the present disclosure. In certain embodiments, an HSV antigen construct can include and/or encode one or more HSV antigens including one or more T cell antigens for HSV of the present disclosure and one or more HSV antigens that is a B cell antigen for HSV (e.g., an antigen that is or includes a B cell epitope disclosed herein or otherwise known in the art). In certain embodiments, an HSV antigen construct can include and/or encode one or more HSV antigens including one or more T cell antigens for HSV of the present disclosure and one or more HSV antigens selected from HSV glycoproteins or fragments thereof. In certain embodiments, an HSV antigen construct can include and/or encode one or more HSV antigens including one or more T cell antigens for HSV of the present disclosure and one or more HSV antigens selected from an HSV gD protein or an antigenic fragment thereof, an HSV gB protein or an antigenic fragment thereof, an HSV gE protein or an antigenic fragment thereof, an HSV gG protein or an antigenic fragment thereof, an HSV gI protein or an antigenic fragment thereof, an HSV gH protein or an antigenic fragment thereof, an HSV gL protein or an antigenic fragment thereof, an HSV ICP4 protein or an antigenic fragment thereof, or an ICP8 protein or an antigenic fragment thereof.

In various embodiments, an HSV antigen construct can be present in a composition for delivery of the HSV antigen construct to a subject. In various embodiments, an HSV antigen construct can be present in a composition for delivery of one or more HSV antigens and/or epitopes to a subject. In various embodiments, an HSV antigen construct can be or include an RNA molecule that encodes one or more antigens and/or epitopes.

Compositions for delivery of HSV antigen constructs and/or HSV antigen constructs can, in certain embodiments, advantageously include, for example, one or more B cell antigens for HSV and one or more T cell antigens (e.g., CD4 and/or CD8 T cell antigens) for HSV. Without wishing to be bound by any particular scientific theory, and without suggesting other embodiments are also advantageous, combination of B cell antigens and T cell antigens can be advantageous in promoting immune system defenses against HSV at multiple lifecycle points include without limitation prior to cellular entry and after cellular entry.

Among other things, the present disclosure provides an insight that many prior strategies for developing pharmaceutical compositions (e.g., immunogenic compositions, e.g., vaccines) for treatment of and/or protection from viral infection have focused primarily, or even almost exclusively, on development of neutralizing antibodies that target surface glycoproteins. The present disclosure identifies a problem with such strategies including, for example, that they may fail to appreciate value or even criticality of ensuring that an induced immune response includes significant T cell activity (in some embodiments, CD4 T cell activity, in some embodiments CD8 T cell activity, in some embodiments, both).

Alternatively or additionally, the present disclosure provides an insight that consideration of expression of HSV proteins (e.g., at particular periods of the HSV life cycle and/or in particular tissues or compartments of an infected subject) can improve vaccine effectiveness.

In some embodiments, the present disclosure provides technologies for identifying, selecting, and/or characterizing HSV protein sequences (e.g., HSV-1 protein sequences, HSV-2 protein sequences, or a combination thereof), and combinations thereof, particularly useful for inclusion in a pharmaceutical compositions (e.g., immunogenic compositions, e.g., vaccines) as described herein.

In some embodiments, pharmaceutical compositions (e.g., immunogenic compositions, e.g., vaccines) that comprise or deliver CD4 and CD8 antigen(s) of one or more HSV proteins (e.g., HSV-1 proteins, HSV-2 proteins, or a combination thereof), e.g., in addition to one or more B cell antigens. Among other things, the present disclosure provides HSV antigen constructs (e.g., HSV-1 antigen constructs, HSV-2 antigen constructs, or a combination thereof) and compositions (e.g., pharmaceutical compositions, e.g., immunogenic compositions, e.g., vaccines) that comprise and/or deliver antigen constructs that induce both neutralizing antibodies and T cells (e.g., CD4 and/or CD8 T cells). Such neutralizing antibodies and T cells (e.g., CD4 and/or CD8 T cells) can target, for example, an HSV glycoprotein and, in some embodiments, one or more additional HSV proteins. In some embodiments, the present disclosure provides such constructs and compositions that induce particularly strong neutralizing antibody responses and/or particularly diverse T cell responses (e.g., targeting multiple T cell antigens).

In some embodiments, the present disclosure provides such constructs and compositions that induce robust B cell responses. In some embodiments, a B cell response includes the production of a diverse, specific repertoire of antibodies.

In some embodiments, the present disclosure provides such constructs and compositions that induce T cell and B cell responses to HSV antigens and/or epitopes.

The present disclosure provides the recognition, for example, that constructs and compositions comprising RNA molecules as described herein (e.g., encoding for one or more HSV (e.g., HSV-1 and/or HSV-2) antigens and/or epitopes) may result in a higher degree of antigen presentation to various immune system components and/or pathways. In some embodiments, administration of such constructs or compositions may induce T cell and/or B cell responses. The present disclosure provides the insight that, e.g., in some embodiments in which T cell and B cell responses are induced in a subject, the subject may have a more sustained, long-term immune response. Such an immune response can be beneficial, e.g., for preventing HSV (e.g., HSV-1 and/or HSV-2) reactivation with a single administration, which may increase vaccination rates and subject compliance as compared with presently available vaccines that require dosing every few years. In some embodiments, constructs and compositions comprising RNA molecules as described herein (e.g., encoding for one or more HSV (e.g., HSV-1, HSV-2, or a combination thereof) antigens and/or epitopes) can provide more diverse protection (e.g., protection against HSV (e.g., HSV-1 and/or HSV-2) variants) because, without wishing to be bound to any particular theory, the constructs and compositions can induce multiple immune system responses.

The present disclosure also provides the recognition that, by administering constructs and compositions that encode HSV (e.g., HSV-1 and/or HSV-2) antigens and/or epitopes, the constructs and compositions described herein avoid administering HSV (e.g., HSV-1 and/or HSV-2) virions, which may infect the subject, go into latency, and reactivate to cause a flare-up.

Still further, the present disclosure provides an insight (and also identifies a source of a problem in certain prior HSV vaccination strategies) that, in some embodiments, particularly effective pharmaceutical compositions (e.g., immunogenic compositions, e.g., vaccines) alter one or more characteristics of the innate immune system. The present disclosure provides certain such compositions, including, for example, compositions that comprise RNA construct(s) encoding HSV (e.g., HSV-1 and/or HSV-2) protein(s) (e.g., HSV antigens or HSV epitopes) as described herein. Separately, in some embodiments, the present disclosure provides particular pharmaceutical composition (e.g., immunogenic composition, e.g., vaccine) formats including, for example, RNA pharmaceutical compositions (e.g., immunogenic compositions, e.g., vaccines) comprising particular elements and/or sequences useful for vaccination.

The present disclosure provides a variety of insights and technologies related to such HSV (e.g., HSV-1 and/or HSV-2) antigen constructs and vaccine (e.g., RNA vaccine) compositions.

As described herein, in many embodiments, provided compositions (e.g. pharmaceutical compositions, e.g., immunogenic compositions, e.g., vaccines) include an RNA active encoding one or more HSV (e.g., HSV-1 and/or HSV-2) polypeptides or antigenic fragments thereof; in some embodiments such RNA active is a modified RNA format in that its uridine residues are substituted with uridine analog(s) such as pseudouridine; alternatively or additionally, in some embodiments, such RNA active includes particular elements (e.g., cap, 5′UTR, 3′UTR, polyA tail, etc) and/or characteristics (e.g., codon optimization) identified, selected, characterized, and/or demonstrated to achieve significant (e.g., elevated) translatability (e.g., in vitro) and/or expression (i.e., in a subject to whom it has been administered) of encoded protein(s). Still further alternatively or additionally, in some embodiments, such RNA active includes particular elements and/or characteristics identified, selected, characterized, and/or demonstrated to achieve significant RNA stability and/or efficient manufacturing, particularly at large scale (e.g., 0.1-10 g, 10-500 g, 500 g-1 kg, 750 g-1.5 kg; those skilled in the art will appreciate that different products may be manufactured at different scales, e.g., depending on patient population size). In some embodiments, such RNA manufacturing scale may be within a range of about 0.01 g/hr RNA to about 1 g/hr RNA, 1 g/hr RNA to about 100 g/hr RNA, about 1 g RNA/hr to about 20 g RNA/hr, or about 100 g RNA/hr to about 10,000 g RNA/hr. In some embodiments, such RNA manufacturing scale may be tens or hundreds of milligrams to tens or hundreds of grams (or more) of RNA per batch. In some embodiments, such RNA manufacturing scale may allow a batch size within a range of about 0.01 g to about 500 g RNA, about 0.01 g to about 10 g RNA, about 1 g to about 10 g RNA, about 10 g to about 500 g RNA, about 10 g to about 300 g RNA, about 10 g to about 200 g RNA or about 30 g to about 60 g RNA.

Still further, in many embodiments, provided compositions (e.g., pharmaceutical compositions, e.g., immunogenic compositions, e.g., vaccines) that include an RNA active are prepared, formulated, and/or utilized in particular LNP compositions, as described herein.

Among other things, the present disclosure provides technologies for rapid development of a pharmaceutical composition (e.g., immunogenic composition, e.g., HSV vaccine) for delivering particular HSV (e.g., HSV-1 and/or HSV-2) antigen constructs to a subject.

Additionally, the present disclosure provides, for example, nucleic acid constructs encoding HSV (e.g., HSV-1 and/or HSV-2) antigens as described herein, expressed HSV (e.g., HSV-1 and/or HSV-2) proteins, and various methods of production and/or use relating thereto, as well as compositions developed therewith and methods relating thereto.

For example, the present disclosure provides technologies for preventing, characterizing, treating, and/or monitoring HSV (e.g., HSV-1 and/or HSV-2) outbreaks and/or infections including, as noted, various nucleic acid constructs and encoded proteins, as well as agents (e.g., antibodies) that bind to such proteins, and compositions that comprise and/or deliver them.

In some aspects, provided herein are technologies (e.g., compositions and methods) for augmenting, inducing, promoting, enhancing and/or improving an immune response against HSV (e.g., HSV-1 and/or HSV-2) or a component thereof (e.g., a protein or fragment thereof). In some embodiments, technologies provided herein are designed to augment, induce, promote, enhance and/or improve immunological memory against HSV (e.g., HSV-1 and/or HSV-2) or a component thereof (e.g., a protein or fragment thereof). In some embodiments, technologies described herein are designed to act as an immunological boost to a primary vaccine, such as a vaccine directed to an antigen and/or epitopes of HSV (e.g., HSV-1 and/or HSV-2). In some embodiments, compositions of the present disclosure comprise one or more polynucleotide constructs (e.g., one or more string constructs) that encode one or more antigens from HSV (e.g., HSV-1 and/or HSV-2). In some embodiments, the present disclosure provides vaccines or other compositions comprising nucleic acids encoding such HSV (e.g., HSV-1 and/or HSV-2) antigens; those skilled in the art will appreciate from context when reference to a particular polynucleotide (e.g., a DNA or RNA) as “encoding” such antigens in fact is referencing a coding strand or its complement.

The present disclosure provides pharmaceutical compositions (e.g., immunogenic compositions, e.g., vaccines) that deliver particular HSV antigen constructs to a subject (e.g., a patient) and related technologies (e.g., methods). In some embodiments, the present disclosure provides pharmaceutical compositions (e.g., immunogenic compositions, e.g., vaccines) that deliver particular HSV-1 antigen constructs to a subject (e.g., a patient) and related technologies (e.g., methods). In some embodiments, the present disclosure provides pharmaceutical compositions (e.g., immunogenic compositions, e.g., vaccines) that deliver particular HSV-2 antigen constructs to a subject (e.g., a patient) and related technologies (e.g., methods). In some embodiments, the present disclosure provides pharmaceutical compositions (e.g., immunogenic compositions, e.g., vaccines) that deliver particular HSV-1 and HSV-2 antigen constructs to a subject (e.g., a patient) and related technologies (e.g., methods).

The present disclosure further provides the recognition that some HSV antigens are common to both HSV-1 and HSV-2. The present disclosure also provides the recognition that some HSV antigens include sequences conserved between HSV-1 and HSV-2. In addition, the present disclosure recognizes that some HSV-1 antigens have, e.g., at least 75%, at least 80%, at least 85%, at least 90%, at least 91%, at least 92%, at least 93%, at least 94%, at least 95%, at least 96%, at least 97%, at least 98%, or at least 99% identity to comparable HSV-2 antigens.

In some embodiments, the present disclosure provides certain HSV antigen constructs particularly useful in effective vaccination. In some embodiments, HSV antigen constructs are HSV-1 antigen construct, HSV-2 antigen constructs, or a combination thereof.

Antigens utilized in accordance with the present disclosure are or include HSV (e.g., HSV-1 and/or HSV-2) components (e.g., antigenic fragments thereof, including epitopes that may comprise non-amino acid, e.g., carbohydrate moieties), which components induce immune responses when administered to humans (or other animals such as rodents and non-human primates susceptible to HSV (e.g., HSV-1 and/or HSV-2) infection).

In many embodiments, antigens utilized in provided pharmaceutical compositions (e.g., immunogenic compositions, e.g., vaccines) include both B-cell and T-cell antigens and/or epitopes, as described herein. In some particular embodiments, delivered antigens include both B-cell and T cell (e.g., CD4 and/or CD8 T cell) antigens and/or epitopes, optionally together in a single antigen polypeptide. In some embodiments, antigens utilized in provided pharmaceutical compositions (e.g., immunogenic compositions, e.g., vaccines) include T cell antigens and/or epitopes. In some embodiments, antigens utilized in provided pharmaceutical compositions (e.g., immunogenic composition, e.g., vaccine), together, include B cell, CD4 T cell and CD8 T cell epitopes. Indeed, in some embodiments, the present disclosure defines particularly useful epitopes for inclusion in HSV (e.g., HSV-1 and/or HSV-2) vaccines, and/or provides antigens that include them.

Exemplary antigens and/or epitopes for use in compositions described herein included those provided in, e.g., Tables 3-5 herein and antigenic fragments thereof. In some embodiments, exemplary antigens disclosed in Tables 3-5, and/or fragments and/or epitopes thereof, can be useful for compositions described herein.

In some embodiments, a provided pharmaceutical composition (e.g., immunogenic composition, e.g., HSV (e.g., HSV-1 and/or HSV-2) vaccine) comprises or delivers (e.g., causes expression of in a recipient organism, for example by administration of a nucleic acid construct, such as an RNA construct as described herein, that encodes it) an antigen that is or comprises one or more epitopes (e.g., one or more B-cell and/or one or more T-cell antigens and/or epitopes) of an HSV (e.g., HSV-1 and/or HSV-2) protein. In some embodiments, a pharmaceutical composition described herein induces a relevant immune response effective against HSV (e.g., by targeting an HSV-1 protein, an HSV-2 protein, or a combination thereof).

In some embodiments, a provided pharmaceutical composition (e.g., immunogenic composition, e.g., HSV (e.g., HSV-1 and/or HSV-2) vaccine) comprises or delivers an antigen that is or comprises a full-length HSV (e.g., HSV-1 and/or HSV-2) protein. In some embodiments, a provided pharmaceutical composition (e.g., immunogenic composition, e.g., HSV (e.g., HSV-1 and/or HSV-2) vaccine) comprises or delivers an antigen that is or comprises a fragment of an HSV (e.g., HSV-1 and/or HSV-2) protein that is less than a full-length HSV (e.g., HSV-1 and/or HSV-2) protein. In some embodiments, a provided pharmaceutical composition (e.g., immunogenic composition, e.g., HSV (e.g., HSV-1 and/or HSV-2) vaccine) comprises or delivers a chimeric polypeptide that is or comprises part or all of an HSV (e.g., HSV-1 and/or HSV-2) protein and one or more heterologous polypeptide elements.

In some embodiments, an antigen that is included in and/or delivered by a provided pharmaceutical composition (e.g., immunogenic composition, e.g., HSV (e.g., HSV-1 and/or HSV-2) vaccine) is or comprises one or more peptide fragments of an HSV (e.g., HSV-1 and/or HSV-2) antigen; in some such embodiments, each of the one or more peptide fragments includes at least one epitope (e.g., one or more B cell epitopes and/or one or more T cell epitopes), for example as may be predicted, selected, assessed and/or characterized as described herein.

In some embodiments, an antigen that is included in and/or delivered by a provided pharmaceutical composition (e.g., immunogenic composition, e.g., HSV (e.g., HSV-1 and/or HSV-2) vaccine) is or comprises a plurality of peptide fragments of one or more HSV (e.g., HSV-1 and/or HSV-2) antigens. In some embodiments, a single polypeptide antigen may include a plurality of such fragments, e.g., presented as a string of antigens or fragments thereof as described herein (e.g., in that a single polypeptide includes a plurality of amino acid sequences derived from distinct HSV antigens or fragments thereof, optionally separated by or otherwise associated with amino acid linkers or other intervening or terminal amino acid sequences). In some embodiments, a single RNA antigen construct may include a plurality of sequences encoding HSV antigens, e.g., presented as a string of antigen encoding sequences as described herein (e.g., in that a single RNA molecule includes a plurality of nucleic acid sequences encoding distinct HSV antigens or fragments thereof, optionally separated by or otherwise associated with nucleic acid linkers or other intervening or terminal nucleic acid sequences).

In some embodiments, one or more HSV (e.g., HSV-1 and/or HSV-2) antigens or antigenic fragments thereof may be linked with one or more sequences with which it is linked in nature. In some such embodiments, such sequence(s) may be or comprise one or more heterologous elements (e.g., one or more elements, not naturally found in the relevant HSV (e.g., HSV-1 and/or HSV-2) such as a polypeptide or antigenic fragment thereof not naturally found to be directly linked to the relevant HSV (e.g., HSV-1 and/or HSV-2) antigen(s)). For example, in some embodiments, an antigen peptide provided and/or utilized in accordance with the present disclosure may include one or more linker elements and/or one or more membrane association elements and/or one or more secretion elements, etc. In some embodiments, an antigenic polypeptide may comprise a plurality of HSV (e.g., HSV-1 and/or HSV-2) protein fragments or epitopes separated from one another by linkers.

In some embodiments, an HSV (e.g., HSV-1 and/or HSV-2) polypeptide, or fragment or epitope thereof, utilized in a construct as described herein (or encoded by a polyribonucleotide describe herein) may include one or more sequence alterations relative to a particular reference HSV (e.g., HSV-1 and/or HSV-2) polypeptide, or fragment or epitope thereof. For example, in some embodiments, a utilized antigen may include one or more sequence variations found in circulating strains or predicted to arise, e.g., in light of assessments of sequence conservation and/or evolution of HSV (e.g., HSV-1 and/or HSV-2) polypeptides over time and/or across strains. Alternatively or additionally, in some embodiments, a utilized antigen may include one or more sequence variations selected, for example, to impact stability, folding, processing and/or display of the antigen or any epitope thereof.

In some embodiments, an HSV (e.g., HSV-1 and/or HSV-2) polypeptide, or fragment or epitope thereof, utilized in an antigen as described herein shows at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with a relevant corresponding reference (e.g., wild type) polypeptide, fragment or epitope. In some embodiments, an HSV (e.g., HSV-1 and/or HSV-2) polypeptide, or fragment or epitope thereof, utilized in an antigen as described herein shows at least 80%, 81%, 82%, 83%, 84%, 85%, 86%, 87%, 88%, 89%, 90%, 91%, 92%, 93%, 94%, 95%, 96%, 97%, 98%, 99%, or 100% sequence homology (i.e., identity or conservative substitution as is understood in the art) amino acid sequence identity with a relevant corresponding reference (e.g., wild type) protein, fragment or epitope. Moreover, in some embodiments, an HSV (e.g., HSV-1 and/or HSV-2) polypeptide, or fragment or epitope thereof, utilized in an antigen as described herein shares conserved amino acid residues (e.g., at corresponding positions) with a relevant corresponding reference (e.g., wild type) polypeptide, fragment or epitope. Those skilled in the art will appreciate that, in general, lower percent identity or homology may be tolerated for shorter peptides, as a single change will by definition have a larger impact on percent identity or homology when considered relative to a smaller number of residues. For example, those skilled in the art will appreciate that, for sequences longer than about 20 amino acids, percent identity or homology is typically greater than about 80%; for sequences longer than about 50 amino acids, percent identity or homology is typically greater than about 90%.

In some embodiments, assessments of degree of conservation may consider the physiochemical difference between two amino acids as described, for example, in WO2014/180569, which is incorporated herein by reference in its entirety. It is well known in molecular evolution that amino acids that interchange frequently are likely to have chemical and physical similarities whereas amino acids that interchange rarely are likely to have different physico-chemical properties. The likelihood for a given substitution to occur in nature compared with the likelihood for this substitution to occur by chance can measured by log-odds matrices. The patterns observed in log-odds matrices imposed by natural selection “reflect the similarity of the functions of the amino acid residues in their weak interactions with one another in the three dimensional conformation of proteins” (see Dayhoff et al. Atlas of protein sequence and structure 5:345, 1978180569, which is incorporated herein by reference in its entirety). In some embodiments, evolutionary based log-odds matrices, which may be referred to as “T scores” can be used to reflect extent to which a sequence variation might impact T cell recognition. Substitutions with positive T scores (i.e., log-odds) are likely to occur in nature, and hence correspond to two amino acids that have similar physico-chemical properties. Substitutions with positive T scores would have a lower likelihood of altering immunogenicity. Conversely, substitutions with negative T scores reflect substitutions that are unlikely to occur in nature and hence correspond to two amino acids that have significantly different physico-chemical properties. Such substitutions would have a greater chance of altering immunogenicity. In some embodiments, presence of negative T score substitutions within a sequence, even if it is otherwise highly conserved, may indicate that it would be relatively less useful in a vaccine antigen as described herein.

In some embodiments, a utilized antigen induces an immune response that targets a HSV envelope glycoprotein. In some embodiments, one or more antigens induce an immune response that targets a HSV envelope glycoprotein. In some embodiments, one or more antigens comprises one or more HSV protein sequences (e.g., conserved sequences and/or sequences that are or comprise one or more B cell epitopes and/or one or more CD4 epitopes and/or one or more CD8 epitopes) of an antigen or epitope of a HSV envelope glycoprotein. In some embodiments, one or more antigens is or comprises a HSV gD protein or a fragment or epitope thereof. In some embodiments, one or more antigens is or comprises a HSV gB protein or a fragment or epitope thereof. In some embodiments, one or more antigens is or comprises a HSV gE protein or a fragment or epitope thereof. In some embodiments, one or more antigens is or comprises a HSV gG protein or a fragment or epitope thereof. In some embodiments, one or more antigens is or comprises a HSV gI protein or a fragment or epitope thereof. In some embodiments, one or more antigens is or comprises a HSV gE protein or a fragment or epitope thereof. In some embodiments, one or more antigens is or comprises a HSV gH protein or a fragment or epitope thereof. In some embodiments, one or more antigens is or comprises a HSV gL protein or a fragment or epitope thereof. In some embodiments, one or more antigens is or comprises a HSV ICP4 protein or a fragment or epitope thereof. In some embodiments, one or more antigens is or comprises an ICP8 polypeptide, fragment, or epitope thereof.

In various embodiments, an HSV antigen construct includes and/or encodes a plurality of HSV antigens (e.g., a plurality of HSV antigens that are or include one or more T cell antigens for HSV) provided in Table 3, or fragments thereof. In certain embodiments, an HSV antigen construct can include and/or encode at least one HSV antigen provided in Table 3, or fragments thereof. In certain embodiments, an HSV antigen construct can include and/or encode at least one HSV antigen provided in Table 4, or fragments thereof. In certain embodiments, an HSV antigen construct can include and/or encode at least one HSV antigen provided in Table 5, or fragments thereof.

In certain embodiments, an HSV antigen construct can include and/or encode at least one T cell antigen for HSV provided in Table 3, or fragments thereof. In certain embodiments, an HSV antigen construct can include and/or encode at least one T cell antigen for HSV provided in Table 4, or fragments thereof. In certain embodiments, an HSV antigen construct can include and/or encode at least one T cell antigen for HSV provided in Table 5, or fragments thereof.

In certain embodiments, an HSV antigen construct can include and/or encode a plurality of (e.g., 1, 2, 3, 4, 5, 6, 7, 8, 9, 10, 11, 12, 13, 14, 15, 16, 17, 18, or 19) HSV antigens provided in Table 3, or fragments thereof. In certain embodiments, an HSV antigen construct can include and/or encode a plurality of (e.g., 1, 2, 3, 4, 5, 6, 7, 8, 9, or 10) HSV antigens provided in Table 4, or fragments thereof. In certain embodiments, an HSV antigen construct can include and/or encode a plurality of (e.g., 1, 2, 3, 4, 5, 6, 7, 8, or 9) HSV antigens provided in Table 5, or fragments thereof.

In certain embodiments, an HSV antigen construct can include and/or encode a plurality of (e.g., 1, 2, 3, 4, 5, 6, 7, 8, 9, 10, 11, 12, 13, 14, 15, 16, 17, 18, or 19) T cell antigens for HSV selected from HSV antigens provided in Table 3, or fragments thereof. In certain embodiments, an HSV antigen construct can include and/or encode a plurality of (e.g., 1, 2, 3, 4, 5, 6, 7, 8, 9, or 10) T cell antigens for HSV selected from antigens provided in Table 4, or fragments thereof. In certain embodiments, an HSV antigen construct can include and/or encode a plurality of (e.g., 1, 2, 3, 4, 5, 6, 7, 8, or 9) T cell antigens for HSV selected from antigens provided in Table 5, or fragments thereof.

In some embodiments, an antigen utilized in accordance with the present disclosure includes HSV (e.g., HSV-1 and/or HSV-2) protein sequences identified and/or characterized by one or more of:
1) HLA-I or HLA-II binding (e.g., to HLA allele(s) present in a relevant population)
2) HLA ligandomics data, optionally confirmed by mass spectrometry
3) Relatively high expression
4) Sequence conservation
5) Surface exposure
6) Serum reactivity
7) Immunogenicity (e.g., presence of one or more B-cell and/or T-cell antigens and/or epitopes; evidence of ability to induce sterile protection in model systems including, e.g., humans, non-human primates, and/or mice).
8) Absence of sequences that overlap with human proteome

In some embodiments, such characteristics are experimentally or computationally assessed. In some embodiments, such characteristics are assessed by consultation with published reports.

For example, in some embodiments, HLA-I and/or HLA-II binding is experimentally assessed; in some embodiments it is predicted.

In some embodiments, predicted HLA-I or HLA-II binding is assessed using an algorithm such as neonmhc 1 and/or neonmhc2, which predict and/or characterize likelihood of MHC class I and MHC class II binding, respectively. Alternatively or additionally, in some embodiments, an MHC-peptide presentation prediction algorithm or MHC-peptide presentation predictor is or comprises NetMHCpan or NetMHCIIpan. In some embodiments, a hidden markov model approach may be utilized for MHC-peptide presentation prediction and/or characterization. In some embodiments, the peptide prediction model MARIA may be utilized. In some embodiments, NetMHCpan is not utilized to predict or characterize likelihood of MHC binding for peptides as described herein. In some embodiments, the peptide prediction model MARIA may be utilized. In some embodiments, NetMHCIIpan is not utilized to predict or characterize likelihood of MHC binding for peptides as described herein. In some embodiments, neither NetMHCpan nor NetMHCIIpan is utilized to predict or characterize likelihood of MHC binding for peptides as described herein. In some embodiments, an MHC-peptide presentation prediction algorithm or MHC-peptide presentation predictor is or comprises RECON® (Real-time Epitope Computation for ONcology), which offers high quality MHC-peptide presentation prediction based on expression, processing and binding capabilities. See, for example, Abelin et al., Immunity 21:315, 2017; Abelin et al., Immunity 15:766, 2019.

In some embodiments, HLA binding and/or ligandomics assessments will consider the geographic region of subjects to be immunized. For example, in some embodiments, HLA allelic diversity will be considered. In some embodiments, antigen(s) included in a provided pharmaceutical composition (e.g., immunogenic composition, e.g., vaccine) will be or comprise peptides (e.g., epitopes) expected or determined, when considered together, to bind to a significant percentage (e.g., at least 60%, 65%, 70%, 75%, 80%, 85%, 90%, 95%, or more) of HLA alleles expected or known to be present in a relevant region or population. In some embodiments, antigen(s) included in a provided pharmaceutical composition (e.g., immunogenic composition, e.g., vaccine) will be or comprise peptides expected or determined, when considered together, to bind to the most prevalent (e.g., the 1, 2, 3, 4, 5, 6, 7, 8, 9, or 10 most prevalent, or at least 1, 2, 3, 4, or 5 of the 10 most prevalent, etc.) HLA alleles expected or known to be present in a relevant region or population).

In some embodiments, expression level is experimentally determined (e.g., in a model system or in infected humans). In some embodiments, expression level is a reported level (e.g., in a published or presented report). In some embodiments, expression level is assessed as RNA (e.g., via RNASeq). In some embodiments (and typically preferably), expression levels is assessed as protein.

In some embodiments, sequence conservation is assessed, for example, using publicly available sequence evaluation software (such as, for example, multiple sequence alignment programs MAFFT, Clustal Omega, etc.). In some embodiments, sequence conservation is determined by consultation with published resources (e.g., sequences). In some embodiments, sequence conservation includes consideration of currently or recently detected strains (e.g., in an active outbreak).

In some embodiments, surface exposure is assessed by reference to publicly available database and/or software.

In some embodiments, serum reactivity is assessed by contacting serum samples from infected individuals with polypeptides including sequences of interest (e.g., as may be displayed via, for example, phage display or peptide array, etc; see, for example, Whittemore et al “A General Method to Discover Epitopes from Sera” PlosOne, 2016; https://doi.org/10.1371/journal.pone.0157462). In some embodiments, serum reactivity is assessed by consultation with literature reports and or database data indicating serum-recognized sequences.

In some embodiments, assessment of immunoreactivity and/or of presence of an epitope may be or comprise consultation with the Immune Epitope Database (IEDB) which those skilled in the art will be aware is a freely available resource funded by NIAID that catalogs experimental data on antibody and T cell epitopes (see iedb.org).

In some embodiments, antigen(s) utilized in accordance with the present disclosure are characterized by dendritic cell presentation which, in turn may be indicative of HLA binding and/or of immunogenicity.

In some embodiments, antigen(s) utilized in accordance with the present disclosure are or comprises sequences (e.g., epitopes, fragments, complete proteins) of HSV proteins found in the HSV envelope. In some embodiments, antigen(s) utilized in accordance with the present disclosure are or comprises sequences (e.g., epitopes, fragments, complete proteins) of HSV proteins found in the HSV tegument.

Among other things, the present disclosure provides an insight that, in some embodiments, it may be desirable to include two or more different epitopes, optionally from two or more different HSV (e.g., HSV-1 and/or HSV-2) proteins, in pharmaceutical composition (e.g., immunogenic composition, e.g., vaccine) compositions, which can be useful in the treatment of HSV.

TABLE 7
Exemplary antigen fragments

| Antigen | | SEQ ID |
|---|---|---|
| Fragment | Sequence (Amino Acid) | NO: |
| RL2 | CTDEIAPPLRCQSFPCLHPFCIPCMKTWIPLRNTCPLCNTPVAYLI | 174 |
| Fragment | VGVTASGSFSTIPIVNDPRTRVEAEAAVRAGTAVDFIWTGNPRT | |
| | APRSLS | |
| RL2 | LPIAGVSSVVALAPYVNKTVTGDCLPVLDMETGHIGAYVVLVDQ | 175 |
| Fragment | TGNVADLLRAAAPAWSRRTLLPEHARNCVRPPDYPTPPASEWN | |
| | SLWMTPVGNMLFDQGTLVG | |
| RS1 | RAAAWMRQVPDPEDVRVVILYSPLPGEDLAAGRAGGGPPPEWS | 176 |
| Fragment | AERGGLSCLLAALGNRLCGPATAAWAGNWTGAPDVSALGAQGV | |
| | LLLSTRDLAFAGAVEFLGLLAGACDRRLIVVNAVRAADWPADGP | |
| | VVSRQHAYLACEVLPAVQCAVRWPAARDLRRTVLASGRVFGPG | |
| | VFARVEAAHARLYPDAPPLRLCRGANVRYRVRTRFGPDTLVPM | |
| | SPREYRRAVLPALDGRAAAS | |
| UL54 | ETLVAHGPSLYRTFAANPRAASTAKAMRDCVLRQENLIEALASA | 177 |
| fragment | DETLAWCKMCIHHNLPLRPQDPIIGTAAAVLENLATRLRPFLQC | |
| | YLKARGLCGLDDLCSRRRLSDIKDIASFVLVILARLANRVERGVSE | |
| | IDYTTVGVGAGETMHFYIPGACMAGLIEILDTHRQECSSRVCELT | |
| | ASHTIAPLYVHGKYFYCNSLF | |
| UL29 | REDIETIAFIKRFSLDYGAINFINLAPNNVSELAMYYMANQILRYC | 178 |
| Fragment | DHSTYFINTLTAVIAGSRRPPSVQAAAAWAPQGGAGLEAGARAL | |
| | MDSLDAHPGAWTSMFASCNLLRPVMAARPMVVLGLSISKYYGM | |
| | AGNDRVFQAGNWASLLGGKNACPLLIFDRTRKFVL | |
| UL39 | RTFGSAPRLTEDDFGLLNYALAEMRRLCLDLPPVPPNAYTPYHL | 179 |
| Fragment | REYATRLVNGFKPLVRRSARLYRILGVLVHLRIRTREASFEEWM | |
| | RSKEVDLDFGLTERLREHEAQLMILAQALNPYDCLIHSTPNTLVE | |
| | RGLQSALKYEEFYLKRFGGHYMESVFQMYTRIAGFLA | |
| UL49 | KMTRGAPKASATPATDPARGRRPAQADSAVLLDAPAPTASGRT | 180 |
| Fragment | KTPAQGLAKKLHFSTAPPSPTAPWTPRVAGFNKRVFCAAVG | |
| UL9 | LLNNYDVLVLDEVMSTLGQLYSPTMQQLGRVDALMLRLLRTCP | 181 |
| Fragment | RIIAMDATANAQLVDFLCSLRGEKNVHVVIGEYAMPGFSARRCL | |
| | FLPRLGPEVLQAALRRRGPAGGAPPPDAPPDATFFGELEARLAG | |
| | GDNVCIFSSTVSFAEVVARFCRQFTDRVLLLHSLTPPGDVTTWG | |
| | RYRVVIYTTVVTVGLSFDPPHFDSMFAYVKPMNYGPDMVSVYQS | |
| | LGRVRTLRKGELLIYMDGSGARSEPV | |
| UL30 | ISCLLYDLSTTALEHILLFSLGSCDLPESHLSDLASRGLPAPVVLEF | 182 |
| Fragment | DSEFEMLLAFMTFVKQYGPEFVTGYNIINFDWPFVLTKLTEIYKV | |
| | PLDGYGRMNGRGVFRVWDIGQSHF | |
| UL30 | GLLPCLHVAATVTTIGREMLLATRAYVHARWAEFDQLLADFPE | 183 |
| Fragment | AAGMRAPGPYSM | |
| UL40 | TSQCPDINHLRSLSILNRWLETELVFVGDEEDVSKLSEGELGFYR | 184 |
| Fragment | FLFAFLSAADDLVTENLGGLSGLFEQKDILHYYVEQECIEVVHSR | |
| | VYNIIQLVLFHNNDQARRAYVARTINHPAIRVKVDWLEARVREC | |
| | DSIPEKFILMILIEGVFFAASFAAIAYLRTNNLLR | |
| UL5 | HEFGNLMKVLEYGLPITEEHMQFVDRFVVPESYITNPANLPGWT | 185 |
| Fragment | RLFSSHKEVSAYMAKLHAYLKVTREGEFVVFTLPVLTFVSVKEFD | |
| | EYRRL | |
| UL5 | ELFGEVFESAPFSTYVDNVIFRGCELLTGSPRGGLMSVALQTDNY | 186 |
| Fragment | TLMGYTYTRVFAFAEELRRRHATAGVAEFLEESPLPYIVLRDQH | |
| | GFMSVVNTNI | |
| UL52 | SVAAPVEVTALYATDGCVITSSLALLTNCLLGAEPLYIFSYDAYRS | 187 |
| fragment | DAPNGPTGAPTEQERFEGSRALYRDAGGLNGDSFRVTFCLLGTE | |
| | VGVTHHPKGRTRPMFVCRFERADDVAVLQDALGRGTPLLPAHV | |
| | TATLDLEATFALHANIIMALTVAIVHNAPARIGSGSTAPLYEPGES | |
| | MRSVV | |
| UL1 | RTPADDVSWRYEAPSVIDYARIDGIFLRYHCPGLDTFLWDRHAQ | 188 |
| Fragment | RAYLVNPFLFAAGFLEDLSHSVFPADTQETT | |
| UL19 | DGRLLHNTQARAADAADDRPHRPADWTVHHKIYYYVLVPAFSR | 189 |
| Fragment | GRCCTAGVRFDRVYATLONMVVPEIAPGEECPSDPVTDPAHPLH | |
| | PANLVANTVKRMFHN | |
| UL21 | SPTQKLAVYYYLIHRERRMSPFPALVRLVGRYIQRHGLYVPAPDE | 190 |
| Fragment | PTLADAMNGL | |
| UL27 | NYTEGIAVVFKENIAPYKFKATMYYKDVTVSQVWFGHRYSQFM | 191 |
| Fragment | GIFEDRAPVPFEEV | |
| UL27 | SVYPYDEFVLATGDFVYMSPFYGYREGSH | 192 |
| Fragment | | |
| UL46 | GLASDPHYDYIRHYASAAKQALGEVELSGGQLSRAILAQYWKYL | 193 |
| Fragment | QTVVPSGLDIPDDPAGDCDPSLHVLLRPTLLPKLLVRAPFKSGAA | |
| | AAKYAAAVAGLRDAAHRLQQYMFFMRPADPSRPSTDTALRLSE | |
| | LLAYVSVLYHWASWMLWTADKYV | |
| UL47 | GPDAAVFRSSLGSLLYWPGVRALLGRDCRVAARYAGRMTYIATG | 194 |
| Fragment | ALLARFNPGAVKCVLPREAAFAGRVL | |
| UL48 | ALFNRLLDDLGFSAGPALCTMLDTWNEDLFSGFPTNADMYREC | 195 |
| Fragment | KFLSTLPSDVIDWGDAHVPERSPIDIRAHGDVAFPTLPATRDELP | |
| | SYYEAMAQFFRGELRA | |
| UL25 | FLWEDQTLLRATANTITALAVLRRLLANGNVYADRLDNRLQLG | 196 |
| Fragment | MLIPGAVPAEAIARGASGLDSGAIKSGDNNLEALCVNYVLPLYQA | |
| | DPTVELTQLFPGLAALCL | |

### 1. Exemplary Antigen Formats

In some embodiments, an antigen utilized as described herein is or comprises a full-length viral protein. In some embodiments, an antigen utilized as described herein is or comprises a fragment or domain of a viral polypeptide, or an antigenic fragment thereof. In some embodiments, an antigen utilized as described herein is a membrane-tethered antigen (e.g., an antigenic fragment thereof fused with a membrane-associating moiety, such as for example, a transmembrane moiety). In some embodiments, a provided pharmaceutical composition (e.g., immunogenic composition, e.g., vaccine) comprises or delivers antigen sequences that are or comprise one or more antibody epitopes and/or one or more CD4 T cell and/or CD8 T cell epitopes.

In some embodiments, an antigen utilized as described herein includes one or more variant sequences relative to a relevant reference antigen. For example, in some embodiments, a protease cleavage site is removed or blocked; alternatively or additionally, in some embodiments, a terminally truncated antigen is utilized.

In some embodiments, an antigen utilized as described herein includes a multimerization element (e.g., a heterologous multimerization element).

In some embodiments, an antigen utilized as described herein includes a membrane association element (e.g., a heterologous membrane association element), such as a transmembrane domain.

In some embodiments, an antigen utilized as described herein includes a secretion signal (e.g., a heterologous secretion signal).

In some embodiments, utilized sequences may be longer (and, e.g., may therefore include more epitopes) than a viral protein found in nature.

In some embodiments, utilized sequences may be from a different strain or plurality of strains (e.g., as may be circulating in and/or otherwise relevant to a population to which a pharmaceutical composition (e.g., immunogenic composition, e.g., vaccine) is administered).

In some embodiments, an antigen utilized as described herein may include a plurality of epitopes (e.g., B-cell and/or T-cell antigens and/or epitopes) arranged in a non-natural configuration (e.g., in a string construct as described herein). In some embodiments, an antigen utilized as described herein may include a plurality of epitopes predicted or demonstrated to bind HLA alleles reflective of a population to which a pharmaceutical composition (e.g., immunogenic composition, e.g., vaccine) composition is to be administered as described herein.

In some embodiments, a provided pharmaceutical composition (e.g., immunogenic composition, e.g., vaccine) may comprise or deliver a plurality of antigens. one or more antigens that includes B cell epitopes and one or more antigens that includes T cell epitopes. In some embodiments, a provided pharmaceutical composition (e.g., immunogenic composition, e.g., vaccine) may comprise or deliver one antigen that includes both B cell and CD4 epitopes and a separate antigen that includes CD8 epitopes.

As described herein, in some embodiments, provided technologies involve administration of a plurality of antigens to the same subject. In some embodiments, multiple antigens are administered at the same time (e.g., in a single dose). In some embodiments, different antigens may be administered at different times (for example in different doses—e.g., a prime dose vs a boost dose). In some embodiments, multiple antigens are administered via the same composition.

For clarity, a single “antigen” polypeptide may include multiple “epitopes”, which in turn may or may not be linked with one another in nature. For example, a single string construct antigen includes multiple epitopes, which may be from different parts of the same HSV (e.g., HSV-1 and/or HSV-2) protein and/or from different HSV (e.g., HSV-1 and/or HSV-2) proteins, linked together as described herein in a single polypeptide.

Thus, a single pharmaceutical composition (e.g., immunogenic composition, e.g., vaccine) as described herein may comprise or deliver (e.g., because the pharmaceutical composition includes a nucleic acid, such as an RNA, that encodes the antigen and is expressed upon administration) a single antigen, which itself may comprise multiple epitopes (either in their natural arrangement relative to one another or in an engineered or constructed arrangement as described herein), or may comprise or deliver a plurality of antigens, each of which similarly may be or comprise a single epitope or multiple epitopes (either in their natural arrangement relative to one another or in an engineered or constructed arrangement as described herein). Still further, a single pharmaceutical composition (e.g., immunogenic composition, e.g., vaccine) may, for example, include multiple distinct nucleic acids (e.g., RNAs) that each encode different antigen(s) or, in some embodiments, may include a single nucleic acid that encodes (and expresses) multiple antigens. Yet further, a single pharmaceutical composition (e.g., immunogenic composition, e.g., vaccine) that includes multiple distinct nucleic acids (e.g., RNAs) encoding antigens may, in some embodiments be prepared by mixing the RNAs and then incorporating the mixture into LNPs, or alternatively by formulating individual RNAs into LNPs and then mixing the LNPs. In some embodiments, mixtures (whether of RNAs pre-LNP preparation or of LNPs) may include the relevant RNAs in 1:1 ratio, or in other ratios as may be preferred (e.g., to achieve a desired relative presentation of antigens or epitopes) in a subject to whom the composition is administered.

In some particular embodiments, a pharmaceutical composition (e.g., immunogenic composition, e.g., vaccine) may comprise or deliver a combination comprising a polypeptide or fragment thereof encoded by all or part of UL1, UL21, UL27, UL29, UL39, UL40, UL46, UL47, UL48, UL49, RS1, RL2, UL5, UL9, UL19, UL25, UL30, UL52, US1, US7, US8, UL22, and/or UL54 or fragments thereof.

In some embodiments, a provided composition includes or delivers an HSV (e.g., HSV-1 and/or HSV-2) envelope glycoprotein antigen (e.g., a full-length HSV (e.g., HSV-1 and/or HSV-2) envelope glycoprotein, a fragment thereof, or one or more epitopes thereof, for example in a string construct). In some embodiments, a provided composition includes or delivers such an HSV (e.g., HSV-1 and/or HSV-2) envelope glycoprotein antigen together with one or more B cell targets (e.g., epitopes) which may, for example, be or comprise one or more other HSV (e.g., HSV-1 and/or HSV-2) proteins (or fragments or epitopes thereof). In some embodiments, such a B cell target is or comprises an HSV (e.g., HSV-1 and/or HSV-2) protein (or fragment or epitope thereof) that is predicted or known to induce a B cell response in infected humans. For example, in some embodiments, a B cell target is or comprises an HSV (e.g., HSV-1 and/or HSV-2) protein (or fragment or B cell epitope thereof) against which sera from infected individual(s) is reactive. In some particular embodiments, a B cell target is or comprises an HSV (e.g., HSV-1 and/or HSV-2) envelope glycoprotein, or other relevant HSV (e.g., HSV-1 and/or HSV-2) protein, or a fragment or epitope thereof.

In some embodiments, a provided composition comprises or delivers a string construct antigen that includes a plurality of T cell epitopes, optionally from more than one HSV (e.g., HSV-1 and/or HSV-2) protein. In some such embodiments, a provided composition further comprises or delivers one or more B cell targets. Alternatively or additionally, in some embodiments, a string construct antigen so utilized includes HSV (e.g., HSV-1 and/or HSV-2) sequences (e.g., one or more fragments or epitopes, e.g., T cell epitopes and/or B cell epitopes, but in some embodiments specifically T cell epitopes).

In some embodiments, a string construct antigen includes both B cell epitopes and T cell epitopes (optionally from the same HSV (e.g., HSV-1 and/or HSV-2) protein or from different HSV (e.g., HSV-1 and/or HSV-2) proteins).

In some embodiments, different antigens may be delivered by administration of different compositions, which in turn may, in some embodiments, be administered at the same time (e.g., as an admixture or otherwise substantially simultaneously) and, in some embodiments, may be administered at different times. To give but one example, in some embodiments, a particular antigen or antigen(s) may be delivered via an initial pharmaceutical composition (e.g., immunogenic composition, e.g., vaccine) dose, and one or more other antigen(s) may be delivered via one or more booster dose(s).

### 2. Exemplary Multi-Epitope Antigens

In some embodiments, an antigen utilized (i.e., included in and/or otherwise delivered by) a pharmaceutical composition (e.g., immunogenic composition, e.g., vaccine) described herein comprises multiple epitopes, e.g., of a single HSV (e.g., HSV-1 and/or HSV-2) protein or of multiple proteins.

In some embodiments, an antigen may comprise two or more epitopes from the same HSV (e.g., HSV-1 and/or HSV-2) protein and in their natural configuration relative to one another (e.g., in a fragment if the relevant protein). In some embodiments, however, an antigen may comprise at least two epitopes configured in a non-natural relationship relative to one another (e.g., included in a string construct as described herein).

Among other things, the present disclosure provides an insight that string construct antigens may be particularly useful or effective for vaccination against an HSV (e.g., HSV-1 and/or HSV-2) infection. Without wishing to be bound by any particular theory, the present disclosure proposes that ability to link individual epitopes predicted or determined to have specific attributes—e.g., binding to relevant HLA alleles, expression at relevant times of infection, representation of particularly conserved sequences, potentially across a plurality of different HSV (e.g., HSV-1 and/or HSV-2) proteins, may prove uniquely beneficial, or indeed critical, for effective vaccination against HSV (e.g., HSV-1 and/or HSV-2), where more traditional vaccination approaches have thus far provided only limited protection.

In some embodiments, a multi-epitope antigen (e.g., a string construct antigen or a polyepitopic antigen) may be administered as a polypeptide and/or as a collection of peptides. Alternatively or additionally, a multi-epitope antigen may be administered as preparation of cells that comprise (e.g., express) the antigen. However, the present disclosure further provides an insight that, in some embodiments, delivery by administration of a nucleic acid, and particularly of an RNA, encoding the multi-epitope antigen, may be particularly useful and/or effective.

As noted elsewhere herein, experience with SARS-CoV-2 has demonstrated that RNA administration can be a particularly effective way to deliver an infectious disease antigen. Furthermore, the present disclosure provides an insight that various features of nucleic acid formats including, for example their flexibility and amenability to rapid design and modification, including incorporation of a variety of insights (e.g., bioinformatics inputs etc), renders them particularly attractive for use in an HSV (e.g., HSV-1 and/or HSV-2) vaccine. Among other things, the present disclosure provides an insight that, in some embodiments, administration of an RNA encoding a string construct antigen as described herein may be a particularly desirable and/or effective approach to immunizing against HSV (e.g., HSV-1 and/or HSV-2) infection.

In some embodiments, a “string” polynucleotide sequence encodes a plurality of antigens and/or epitopes in tandem. In some embodiments, a string encodes about 2 to about 100, about 2 to about 75, about 2 to about 50, about 2 to about 25, about 2 to about 20, about 2 to about 15, about 2 to about 10, or about 2 to about 5 antigens and/or epitopes. In some embodiments, a string encodes about 5 to about 100, about 5 to about 75, about 5 to about 50, about 5 to about 25, about 5 to about 20, about 5 to about 15, or about 5 to about 10 antigens and/or epitopes. In some embodiments, a “string” polynucleotide sequence encodes a plurality of epitopes in tandem. In some embodiments, a string encodes about 2 to about 1000 or about 2 to about 10,000 antigens and/or epitopes. In some embodiments about 2-5,000 antigens and/or epitopes are encoded in one polynucleotide string. In some embodiments, about 2-4,000 antigens and/or epitopes are encoded in one polynucleotide string. In some embodiments, about 2-3,000 antigens and/or epitopes are encoded in one polynucleotide string. In some embodiments about 2-2,000 antigens and/or epitopes are encoded in one polynucleotide string. In some embodiments, about 2-1,000 antigens and/or epitopes are encoded in one polynucleotide string. In some embodiments, about 10-500 antigens and/or epitopes are encoded in one polynucleotide string. In some embodiments, about 10-200 antigens and/or epitopes are encoded in one polynucleotide string. In some embodiments, about 20-100 antigens and/or epitopes are encoded in one polynucleotide string.

In some embodiments, epitopes encoded by string constructs comprise epitopes that are predicted by a HLA binding and presentation prediction software to be of high likelihood to be presented by a protein encoded by an HLA to a T cell for eliciting immune response. In some embodiments, epitopes that are predicted to have a high likelihood to be presented by a protein encoded by an HLA, are selected from any one of the proteins or peptides described in Tables 3-5. In some embodiments, epitopes in a string construct comprise membrane-associated or otherwise accessible epitopes, e.g., at relevant time(s) during the HSV (e.g., HSV-1 and/or HSV-2) life cycle.

In some embodiments, an antigen utilized in accordance with the present disclosure an antigen is or comprises UL1, UL21, UL27, UL29, UL39, UL40, UL46, UL47, UL48, UL49, RS1, RL2, UL5, UL9, UL19, UL25, UL30, UL52, US1, US7, US8, UL22, and/or UL54 or fragments thereof, and variants thereof and/or fragments or epitopes of any of the foregoing, and combinations of any of the foregoing. In some embodiments, an antigen utilized in accordance with the present disclosure an antigen is or comprises a HSV protein is or comprises a HSV envelope protein, a HSV tegument protein, a HSV membrane protein, and variants thereof and/or fragments or epitopes of any of the foregoing, and combinations of any of the foregoing. In some embodiments, a string construct may comprise a multitude of epitopes that are from 2, 3, 4, or more HSV proteins. In some embodiments a string constructs comprise one or more features described in herein, including the examples and tables. In some embodiments the String constructs comprise a sequence, or are encoded by a sequence, as depicted in Tables 3-5.

Alternatively or additionally, in some embodiments, one or more string constructs may include one or more other epitopes (e.g., as may be predicted or demonstrated, for example in literature). In some embodiments, a string construct may comprise sequences encoding features such as linkers, and cleavage sites (e.g., auto-cleavage sites such as, for example, T2A, or P2A sequences). In some embodiments, a linker that is enriched in G and S residues can be used. In some embodiments, an exemplary linker may have a sequence of GGGGSGGGGS (SEQ ID NO: 167) or GGSGGGGSGG (SEQ ID NO: 165).

In some embodiments, a string construct comprises two or more overlapping epitope sequences.

In some embodiments, a string construct comprises a sequence, or is encoded by a sequence, that is 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of the sequences in Tables 3-5. As noted above, where sequences being compared are longer than about 20 amino acids, percent identity or homology is typically greater than about 80%; for sequences longer than about 50 amino acids, percent identity or homology is typically greater than about 90%.

In some embodiments, epitopes are arranged on a string to maximize immunogenicity of the string, for example by maximizing recognition by HLA allele repertoire of a subject. In some embodiments, the same string encodes epitopes that can bind to and/or are predicted to bind to different HLA alleles. For instance, as is well exemplified in the sequences tables, e.g., at least in Tables 3-5 a string may encode epitope(s) that comprise: (a) a first epitope that binds to or is predicted to bind to a first MHC peptide encoded by a first HLA allele; (b) a second epitope that binds to or is predicted to bind to a second MHC peptide encoded by a second HLA allele; (c) a third epitope that binds to or is predicted to bind to a third MHC peptide encoded by a third HLA allele—and more such epitopes can be added, as in for example in string sequences as provided herein; wherein the first, second and third epitopes are epitopes from the same HSV (e.g., HSV-1 and/or HSV-2) protein, or from different HSV (e.g., HSV-1 and/or HSV-2) proteins. In this way, epitope distribution encoded by a single string is maximized for hitting the different MHC based presentation to T cells, thereby maximizing the probability of generating a desired immune response from a wider range of patients in the given population and the robustness of the response of each patient.

In some embodiments, epitopes included in a string construct are selected on the basis of high scoring prediction for binding to an HLA by a reliable prediction algorithm or system, such as the RECON prediction algorithm. In some embodiments, the present disclosure provides an insight that particularly successful strings can be provided by selecting epitopes based on highly reliable and efficient prediction algorithm, in the layout of the epitopes encoded by the string, with or without non-epitope sequences or sequences flanking the epitopes, and is such that the immunogenicity of the string is validated in an ex vivo cell culture model, or in an animal model, specifically in showing T cell induction following vaccination with a string construct or a polypeptide encoded by a string construct with the finding of epitope specific T cell response. In some embodiments, validation may be from using in human patients, and with a finding that T cells obtained from a patient post vaccination shows epitope specific efficient and lasting T cell response. In some embodiments, efficiency of a string as a vaccine is influenced by its design that in part depends on strength of the bioinformatics information used in the thoughtful execution of the design, the reliability of the MHC presentation prediction model, the efficiency of epitope processing when a string vaccine is expressed in a cell, among others.

In some embodiments a multi-epitopic RNA (e.g., mRNA) construct as described above comprises 1, 2, 3, 4, 5, 6, 7, 8, 9, 10, 11, 12, 13, 14, 15 or more antigens and/or epitopes. In some embodiments, a pharmaceutical composition comprises 2, 3, 4, 5, 6, 7, 8, 9, or 10 or more strings. In some embodiments, a pharmaceutical composition comprises 6 strings. In some embodiments, a pharmaceutical composition comprises 7 strings. In some embodiments, a pharmaceutical composition comprises 8 strings. In some embodiments, a pharmaceutical composition comprises 9 strings. In some embodiments, a pharmaceutical composition comprises 10 strings.

In some embodiments, epitope-coding sequences in a string construct are flanked by one or more sequences selected for higher immunogenicity, better cleavability for peptide presentation to MHCs, better expression, and/or improved translation in a cell in a subject. In some embodiments, flanking sequences comprise a linker with a specific cleavable sequences. In some embodiments, epitope-coding sequences in a string construct are flanked by a secretory protein sequence.

In some embodiments, a string sequence encodes an epitope that may comprise or otherwise be linked to a signal sequence, such as those listed in Table 7, or at least a sequence having 1, 2, 3, 4, or 5 amino acid differences relative thereto. In some embodiments, a string sequence encodes an epitope that may comprise or otherwise be linked to a signal sequence such as MFVFLVLLPLVSSQCVNLT (SEQ ID NO: 90), or at least a sequence having 1, 2, 3, 4, or at the most 5 amino acid differences relative thereto. In some embodiments, a string sequence encodes an epitope that may be linked at the N-terminal end by a sequence that is enriched in G and S residues, or a sequence having 1, 2, 3, 4, or at the most 5 amino acid differences relative thereto. In some embodiments, an exemplary linker that may be useful to link epitopes has a sequence of GGSGGGGSGG (SEQ ID NO: 165).

In some embodiments, linked sequences may comprise a linker with a cleavage sequence, e.g., with specific cleavable sequences.

In some embodiments, a string construct is linked to a transmembrane domain (TM) or other membrane-associating element. In some embodiments, a linker may have a length of 2, 3, 4, 5, 6, 7, 8, 9, 10 or more amino acid. In some embodiments, a linker of not more than about 30, 25, 20, 15, 10 or fewer amino acids is used. In some embodiments, a linker sequence is not limited to comprise any particular amino acids; in some embodiments, a linker sequence comprises any amino acids. In some embodiments, a linker or cleavage sequence comprises glycine (G). In some embodiments, a linker or cleavage sequence comprises serine (S). In some embodiments, a linker is designed to comprise amino acids based on a cleavage predictor to generate highly-cleavable sequences peptide sequences, and is a novel and effective way of delivering immunogenic T cell epitopes in a T cell vaccine setting.

In some embodiments, epitope distribution and their juxtaposition encoded in a string construct are so designed to facilitate cleavage sequences contributed by the amino acid sequences of the epitopes and/or the flanking or linking residues and thereby using minimal linker sequences. Some exemplary cleavage sequences, without limitation, may be one or more of FRAC, KRCF, KKRY, ARMA, RRSG, MRAC, KMCG, ARCA, KKQG, YRSY, SFMN, FKAA, KRNG, YNSF, KKNG, RRRG, KRYS, and ARYA (SEQ ID NOs: 62-79, respectively).

In some embodiments, a string construct is RNA (e.g., mRNA). In some embodiments, a pharmaceutical composition comprises one or more RNA (e.g., mRNA) string constructs, each comprising a sequence encoding a plurality of epitopes as described herein. In some embodiments, the one or more RNA (e.g., mRNA) comprises a plurality of epitopes, wherein each of the plurality of epitopes is predicted by an HLA binding and presentation prediction algorithm to be of high likelihood to be presented by a protein encoded by an HLA to a T cell for eliciting immune response.

In some embodiments, one or more RNAs (e.g., mRNAs) utilized in a pharmaceutical composition (e.g., immunogenic composition, e.g., vaccine) as described herein encodes a plurality of epitopes (e.g., including one or more, or two or more, antigens provided in Table 4, Table 5, or Table 6, or fragments thereof or epitopes thereof), optionally wherein each of the plurality is predicted by an HLA binding and presentation prediction algorithm to be of high likelihood to be presented by a protein encoded by an HLA to a T cell for eliciting immune response. In some embodiments, the plurality of epitopes comprises epitopes from a single HSV (e.g., HSV-1 and/or HSV-2) protein. In some embodiments, the plurality of epitopes comprises epitopes from multiple HSV (e.g., HSV-1 and/or HSV-2) proteins.

In some embodiments, one or more RNAs (e.g., mRNAs) utilized in a pharmaceutical composition (e.g., immunogenic composition, e.g., vaccine) as described herein include a first RNA that encodes an HSV (e.g., HSV-1, HSV-2 or both) antigen expressed prior to cell infiltration or infection and includes one or more fragments expected or known to interface with host cytoplasm. In some embodiments, a HSV antigen encoded by a first RNA is or comprises an HSV antigen, fragment, or epitope, e.g., a UL1, UL21, UL27, UL29, UL39, UL40, UL46, UL47, UL48, UL49, RS1, RL2, UL5, UL9, UL19, UL25, UL30, UL52, US1, US7, US8, UL22, and/or UL54 or fragments thereof, epitopes thereof, and/or a combination thereof. In some embodiments, one or more RNAs (e.g., mRNAs) utilized in a pharmaceutical composition (e.g., immunogenic composition, e.g., vaccine) as described herein includes a second antigen RNA that encodes a multi-epitopic (e.g., polyepitopic) antigen. In some embodiments, a multi-epitopic antigen comprises two or more antigens found in Tables 3-5 herein, or fragments thereof or epitopes thereof. In some embodiments, a multi-epitopic antigen comprises two or more antigens listed in Tables 3-5, and/or fragments and/or epitopes thereof.

In some embodiments, one or more RNAs (e.g., mRNAs) utilized in a pharmaceutical composition (e.g., immunogenic composition, e.g., vaccine) as described herein includes a plurality of epitopes that are predicted by an HLA binding and presentation prediction algorithm to be of high likelihood to be presented by a protein encoded by an HLA to a T cell for eliciting immune response. In some embodiments, the plurality of epitopes comprises epitopes from a single HSV (e.g., HSV-1, HSV-2 or both) protein. In some embodiments, the plurality of epitopes comprises epitopes from multiple HSV (e.g., HSV-1 and/or HSV-2) proteins.

In some embodiments, the RNA (e.g., mRNA) comprises a 5′UTR and a 3′UTR. In some embodiments, an UTR comprises a poly A sequence. In some embodiments, a poly A sequence comprises between 50-200 nucleotides.

In some embodiments, epitopes encoded in a string construct may be flanked by a signal peptide sequence, e.g., SP1 sequence (HSV-1 gD signal peptide/secretory domain).

In some embodiments, a polynucleotide comprises a dEarI-hAg sequence.

In some embodiments, a poly A tail of a string construct may comprise about 150 A residues. In some embodiments, a poly A tail may comprise 120 residues or less. In some embodiments, a poly A tail of a string construct may comprise about 120 A residues. In some embodiments, a poly A tail of a string construct may comprise about 100 residues. In some embodiments, a poly A tail of a string comprises a “split” or “interrupted” poly A tail (e.g., as described in WO2016/005324).

In some embodiments, a multi-epitope antigen encodes a super-motif-bearing or motif-bearing polypeptide, together with a helper epitope (e.g., a heterologous helper epitope) and an endoplasmic reticulum-translocating signal sequence. See, for example, in An & Whitton J. Virol. 71:2292, 1997; Thomson. et al., J. Immunol. 157:822, 1996; Whitton, et al., J. Virol 67:348, 1993; Hanke, et al., Vaccine 16:426, 1998.

### 3. T Cell Antigens and Related Constructs

In certain embodiments, an HSV antigen construct can include and/or encode at least one T cell antigen (e.g., at least one CD4 and/or CD8 T cell antigen) for HSV selected from UL1, UL21, UL27, UL29, UL39, UL40, UL46, UL47, UL48, UL49, RS1, RL2, UL5, UL9, UL19, UL25, UL30, UL52, US1, US7, US8, UL22, and/or UL54 or fragments thereof. In certain embodiments, an HSV antigen construct can include and/or encode at least one T cell antigen for HSV selected from UL1, UL21, UL27, UL29, UL39, UL40, UL46, UL47, UL48, and/or UL49 or fragments thereof. In certain embodiments, an HSV antigen construct can include and/or encode at least one T cell antigen for HSV selected from RS1, RL2, UL5, UL9, UL19, UL25, UL30, UL52, US1, US7, US8, UL22, and/or UL54 or fragments thereof.

In certain embodiments, an HSV antigen construct can include and/or encode a plurality of (e.g., 1, 2, 3, 4, 5, 6, 7, 8, 9, 10, 11, 12, 13, 14, 15, 16, 17, 18, or 19) T cell antigens (e.g., CD4 and/or CD8 T cell antigens) for HSV selected from UL1, UL21, UL27, UL29, UL39, UL40, UL46, UL47, UL48, UL49, RS1, RL2, UL5, UL9, UL19, UL25, UL30, UL52, US1, US7, US8, UL22, and/or UL54 or fragments thereof. In certain embodiments, an HSV antigen construct can include and/or encode a plurality of (e.g., 1, 2, 3, 4, 5, 6, 7, 8, 9, or 10) T cell antigens for HSV selected from UL1, UL21, UL27, UL29, UL39, UL40, UL46, UL47, UL48, and/or UL49 or fragments thereof. In certain embodiments, an HSV antigen construct can include and/or encode a plurality of (e.g., 1, 2, 3, 4, 5, 6, 7, 8, or 9) T cell antigens for HSV selected from RS1, RL2, UL5, UL9, UL19, UL25, UL30, UL52, US1, US7, US8, UL22, and/or UL54 or fragments thereof.

In some embodiments, a polyribonucleotide according to the present disclosure encodes a polypeptide that comprises two or more HSV antigens or antigenic fragments. In some embodiments, two or more HSV antigenic fragments are each a fragment of a different HSV antigen. In some embodiments, at least two of the HSV antigenic fragments are a fragment from the same HSV antigen.

In some embodiments, a polyribonucleotide encoding a polypeptide, wherein the polypeptide comprises three or more HSV antigens or antigenic fragments thereof. In some embodiments, three or more HSV antigenic fragments are each a fragment of a different HSV antigen. In some embodiments, at least two out of three of the HSV antigenic fragments are a fragment from the same HSV antigen.

In some embodiments, a polyribonucleotide encoding a polypeptide, wherein the polypeptide comprises four or more HSV antigens or antigenic fragments thereof. In some embodiments, four or more HSV antigenic fragments are each a fragment of a different HSV antigen. In some embodiments, at least two out of four of the HSV antigenic fragments are a fragment from the same HSV antigen.

In some embodiments, a polyribonucleotide encoding a polypeptide, wherein the polypeptide comprises five or more HSV antigens or antigenic fragments thereof. In some embodiments, five or more HSV antigenic fragments are each a fragment of a different HSV antigen. In some embodiments, at least two out of five of the HSV antigenic fragments are a fragment from the same HSV antigen.

In some embodiments, a polyribonucleotide encoding a polypeptide, wherein the polypeptide comprises six or more HSV antigens or antigenic fragments thereof. In some embodiments, six or more HSV antigenic fragments are each a fragment of a different HSV antigen. In some embodiments, at least two out of six of the HSV antigenic fragments are a fragment from the same HSV antigen.

In some embodiments, a polypeptide according to the present disclosure does not comprise a full length HSV antigen.

In some embodiments, a polypeptide according to the present disclosure comprises one or more HSV antigens or antigenic fragments thereof that comprise one or more T cell antigens.

In some embodiments, one or more HSV antigens or antigenic fragments thereof have at least 80% sequence identity with one or more sequences selected from SEQ ID NOs: 1-74 or a corresponding fragment thereof.

In some embodiments, an HSV T cell antigen is or includes a UL1 polypeptide or fragment thereof. In various embodiments, a UL1 polypeptide or fragment thereof has at least 80% sequence identity with a UL1 amino acid sequence set forth in Table 3 or otherwise known in the art, or a corresponding fragment thereof. In some embodiments, a UL1 polypeptide or fragment thereof has at least 80%, such as at least 80%, 85%, 90%, 91%, 92%, 93%, 94%, 95%, 96%, 97%, 99%, or 100% sequence identity with a UL1 amino acid sequence set forth in Table 3 or otherwise known in the art, or a corresponding fragment thereof. Examples of UL1 polypeptides known in the art include UL1 polypeptides encoded by known HSV strains such as, without limitation, HG52, G, 333, and MS strains. In some embodiments, a UL1 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 1, 2 and/or 3.

In some embodiments, an HSV T cell antigen is or includes a UL21 polypeptide or fragment thereof. In various embodiments, a UL21 polypeptide or fragment thereof has at least 80% sequence identity with a UL21 amino acid sequence set forth in Table 3 or otherwise known in the art, or a corresponding fragment thereof. In some embodiments, a UL21 polypeptide or fragment thereof has at least 80%, such as at least 80%, 85%, 90%, 91%, 92%, 93%, 94%, 95%, 96%, 97%, 99%, or 100% sequence identity with a UL1 amino acid sequence set forth in Table 3 or otherwise known in the art, or a corresponding fragment thereof. Examples of UL21 polypeptides known in the art include UL21 polypeptides encoded by known HSV strains such as, without limitation, HG52, G, 333, and MS strains. In some embodiments, a UL21 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 4, 5 and/or 6.

The UL27 open reading frame encodes HSV gB (also referred to herein as UL27 polypeptide). In some embodiments, an HSV T cell antigen is or includes a UL27 polypeptide or fragment thereof. In various embodiments, a UL27 polypeptide or fragment thereof has at least 80% sequence identity with a UL27 amino acid sequence set forth in Table 3 or otherwise known in the art, or a corresponding fragment thereof. In some embodiments, a UL27 polypeptide or fragment thereof has at least 80%, such as at least 80%, 85%, 90%, 91%, 92%, 93%, 94%, 95%, 96%, 97%, 99%, or 100% sequence identity with a UL1 amino acid sequence set forth in Table 3 or otherwise known in the art, or a corresponding fragment thereof. Examples of UL27 polypeptides known in the art include UL27 polypeptides encoded by known HSV strains such as, without limitation, HG52, G, 333, and MS strains. In some embodiments, a UL27 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 7, 8, 9 and/or 74.

In some embodiments, an HSV T cell antigen is or includes a UL29 polypeptide or fragment thereof. In various embodiments, a UL29 polypeptide or fragment thereof has at least 80% sequence identity with a UL29 amino acid sequence set forth in Table 3 or otherwise known in the art, or a corresponding fragment thereof. In some embodiments, a UL29 polypeptide or fragment thereof has at least 80%, such as at least 80%, 85%, 90%, 91%, 92%, 93%, 94%, 95%, 96%, 97%, 99%, or 100% sequence identity with a UL1 amino acid sequence set forth in Table 3 or otherwise known in the art, or a corresponding fragment thereof. Examples of UL29 polypeptides known in the art include UL29 polypeptides encoded by known HSV strains such as, without limitation, HG52, G, 333, and MS strains. In some embodiments, a UL29 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 10, 11, and/or 12.

In some embodiments, an HSV T cell antigen is or includes a UL39 polypeptide or fragment thereof. In various embodiments, a UL39 polypeptide or fragment thereof has at least 80% sequence identity with a UL39 amino acid sequence set forth in Table 3 or otherwise known in the art, or a corresponding fragment thereof. In some embodiments, a UL39 polypeptide or fragment thereof has at least 80%, such as at least 80%, 85%, 90%, 91%, 92%, 93%, 94%, 95%, 96%, 97%, 99%, or 100% sequence identity with a UL1 amino acid sequence set forth in Table 3 or otherwise known in the art, or a corresponding fragment thereof. Examples of UL39 polypeptides known in the art include UL39 polypeptides encoded by known HSV strains such as, without limitation, HG52, G, 333, and MS strains. In some embodiments, a UL39 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 13, 14 and/or 15.

In some embodiments, an HSV T cell antigen is or includes a UL40 polypeptide or fragment thereof. In various embodiments, a UL40 polypeptide or fragment thereof has at least 80% sequence identity with a UL40 amino acid sequence set forth in Table 3 or otherwise known in the art, or a corresponding fragment thereof. In some embodiments, a UL1 polypeptide or fragment thereof has at least 80%, such as at least 80%, 85%, 90%, 91%, 92%, 93%, 94%, 95%, 96%, 97%, 99%, or 100% sequence identity with a UL40 amino acid sequence set forth in Table 3 or otherwise known in the art, or a corresponding fragment thereof. Examples of UL40 polypeptides known in the art include UL40 polypeptides encoded by known HSV strains such as, without limitation, HG52, G, 333, and MS strains. In some embodiments, a UL40 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 16, 17 and/or 18.

In some embodiments, an HSV T cell antigen is or includes a UL46 polypeptide or fragment thereof. In various embodiments, a UL46 polypeptide or fragment thereof has at least 80% sequence identity with a UL46 amino acid sequence set forth in Table 3 or otherwise known in the art, or a corresponding fragment thereof. In some embodiments, a UL46 polypeptide or fragment thereof has at least 80%, such as at least 80%, 85%, 90%, 91%, 92%, 93%, 94%, 95%, 96%, 97%, 99%, or 100% sequence identity with a UL1 amino acid sequence set forth in Table 3 or otherwise known in the art, or a corresponding fragment thereof. Examples of UL46 polypeptides known in the art include UL46 polypeptides encoded by known HSV strains such as, without limitation, HG52, G, 333, and MS strains. In some embodiments, a UL46 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 19, 20 and/or 21.

In some embodiments, an HSV T cell antigen is or includes a UL47 polypeptide or fragment thereof. In various embodiments, a UL47 polypeptide or fragment thereof has at least 80% sequence identity with a UL47 amino acid sequence set forth in Table 3 or otherwise known in the art, or a corresponding fragment thereof In some embodiments, a UL47 polypeptide or fragment thereof has at least 80%, such as at least 80%, 85%, 90%, 91%, 92%, 93%, 94%, 95%, 96%, 97%, 99%, or 100% sequence identity with a UL1 amino acid sequence set forth in Table 3 or otherwise known in the art, or a corresponding fragment thereof. Examples of UL47 polypeptides known in the art include UL47 polypeptides encoded by known HSV strains such as, without limitation, HG52, G, 333, and MS strains. In some embodiments, a UL47 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 22, 23 and/or 24.

In some embodiments, an HSV T cell antigen is or includes a UL48 polypeptide or fragment thereof. In various embodiments, a UL48 polypeptide or fragment thereof has at least 80% sequence identity with a UL48 amino acid sequence set forth in Table 3 or otherwise known in the art, or a corresponding fragment thereof. In some embodiments, a UL48 polypeptide or fragment thereof has at least 80%, such as at least 80%, 85%, 90%, 91%, 92%, 93%, 94%, 95%, 96%, 97%, 99%, or 100% sequence identity with a UL1 amino acid sequence set forth in Table 3 or otherwise known in the art, or a corresponding fragment thereof. Examples of UL48 polypeptides known in the art include UL48 polypeptides encoded by known HSV strains such as, without limitation, HG52, G, 333, and MS strains. In some embodiments, a UL48 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 25, 26 and/or 27.

In some embodiments, an HSV T cell antigen is or includes a UL49 polypeptide or fragment thereof. In various embodiments, a UL49 polypeptide or fragment thereof has at least 80% sequence identity with a UL49 amino acid sequence set forth in Table 3 or otherwise known in the art, or a corresponding fragment thereof. In some embodiments, a UL49 polypeptide or fragment thereof has at least 80%, such as at least 80%, 85%, 90%, 91%, 92%, 93%, 94%, 95%, 96%, 97%, 99%, or 100% sequence identity with a UL1 amino acid sequence set forth in Table 3 or otherwise known in the art, or a corresponding fragment thereof. Examples of UL49 polypeptides known in the art include UL49 polypeptides encoded by known HSV strains such as, without limitation, HG52, G, 333, and MS strains. In some embodiments, a UL49 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 28, 29 and/or 30.

In some embodiments, an HSV T cell antigen is or includes a RS1 polypeptide or fragment thereof. In various embodiments, a RS1 polypeptide or fragment thereof has at least 80% sequence identity with a RS1 amino acid sequence set forth in Table 3 or otherwise known in the art, or a corresponding fragment thereof. In some embodiments, a UL1 polypeptide or fragment thereof has at least 80%, such as at least 80%, 85%, 90%, 91%, 92%, 93%, 94%, 95%, 96%, 97%, 99%, or 100% sequence identity with a UL1 amino acid sequence set forth in Table 3 or otherwise known in the art, or a corresponding fragment thereof. Examples of RS1 polypeptides known in the art include RS1 polypeptides encoded by known HSV strains such as, without limitation, HG52, G, 333, and MS strains. In some embodiments, a RS1 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 31, 32 and/or 33.

In some embodiments, an HSV T cell antigen is or includes a RL2 polypeptide or fragment thereof. In various embodiments, a RL2 polypeptide or fragment thereof has at least 80% sequence identity with a RL2 amino acid sequence set forth in Table 3 or otherwise known in the art, or a corresponding fragment. In some embodiments, a RL2 polypeptide or fragment thereof has at least 80%, such as at least 80%, 85%, 90%, 91%, 92%, 93%, 94%, 95%, 96%, 97%, 99%, or 100% sequence identity with a UL1 amino acid sequence set forth in Table 3 or otherwise known in the art, or a corresponding fragment thereof. Examples of RL2 polypeptides known in the art include RL2 polypeptides encoded by known HSV strains such as, without limitation, HG52, G, 333, and MS strains. In some embodiments, a RL2 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 34, 35 and/or 36.

In some embodiments, an HSV T cell antigen is or includes a UL5 polypeptide or fragment thereof. In various embodiments, a UL5 polypeptide or fragment thereof has at least 80% sequence identity with a UL5 amino acid sequence set forth in Table 3 or otherwise known in the art, or a corresponding fragment thereof. In some embodiments, a UL5 polypeptide or fragment thereof has at least 80%, such as at least 80%, 85%, 90%, 91%, 92%, 93%, 94%, 95%, 96%, 97%, 99%, or 100% sequence identity with a UL1 amino acid sequence set forth in Table 3 or otherwise known in the art, or a corresponding fragment thereof. Examples of UL5 polypeptides known in the art include UL5 polypeptides encoded by known HSV strains such as, without limitation, HG52, G, 333, and MS strains. In some embodiments, a UL5 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 37, 38 and/or 39.

In some embodiments, an HSV T cell antigen is or includes a UL9 polypeptide or fragment thereof. In various embodiments, a UL9 polypeptide or fragment thereof has at least 80% sequence identity with a UL9 amino acid sequence set forth in Table 3 or otherwise known in the art, or a corresponding fragment thereof. In some embodiments, a UL9 polypeptide or fragment thereof has at least 80%, such as at least 80%, 85%, 90%, 91%, 92%, 93%, 94%, 95%, 96%, 97%, 99%, or 100% sequence identity with a UL1 amino acid sequence set forth in Table 3 or otherwise known in the art, or a corresponding fragment thereof. Examples of UL9 polypeptides known in the art include UL9 polypeptides encoded by known HSV strains such as, without limitation, HG52, G, 333, and MS strains. In some embodiments, a UL9 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 40, 41 and/or 42.

In some embodiments, an HSV T cell antigen is or includes a UL19 polypeptide or fragment thereof. In various embodiments, a UL19 polypeptide or fragment thereof has at least 80% sequence identity with a UL19 amino acid sequence set forth in Table 3 or otherwise known in the art, or a corresponding fragment thereof. In some embodiments, a UL19 polypeptide or fragment thereof has at least 80%, such as at least 80%, 85%, 90%, 91%, 92%, 93%, 94%, 95%, 96%, 97%, 99%, or 100% sequence identity with a UL1 amino acid sequence set forth in Table 3 or otherwise known in the art, or a corresponding fragment thereof. Examples of UL19 polypeptides known in the art include UL19 polypeptides encoded by known HSV strains such as, without limitation, HG52, G, 333, and MS strains. In some embodiments, a UL19 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 43, 44 and/or 45.

In some embodiments, an HSV T cell antigen is or includes a UL25 polypeptide or fragment thereof. In various embodiments, a UL25 polypeptide or fragment thereof has at least 80% sequence identity with a UL25 amino acid sequence set forth in Table 3 or otherwise known in the art, or a corresponding fragment thereof. In some embodiments, a UL25 polypeptide or fragment thereof has at least 80%, such as at least 80%, 85%, 90%, 91%, 92%, 93%, 94%, 95%, 96%, 97%, 99%, or 100% sequence identity with a UL1 amino acid sequence set forth in Table 3 or otherwise known in the art, or a corresponding fragment thereof. Examples of UL25 polypeptides known in the art include UL25 polypeptides encoded by known HSV strains such as, without limitation, HG52, G, 333, and MS strains. In some embodiments, a UL25 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 46, 47 and/or 48.

In some embodiments, an HSV T cell antigen is or includes a UL30 polypeptide or fragment thereof. In various embodiments, a UL30 polypeptide or fragment thereof has at least 80% sequence identity with a UL30 amino acid sequence set forth in Table 3 or otherwise known in the art, or a corresponding fragment thereof. In some embodiments, a UL30 polypeptide or fragment thereof has at least 80%, such as at least 80%, 85%, 90%, 91%, 92%, 93%, 94%, 95%, 96%, 97%, 99%, or 100% sequence identity with a UL1 amino acid sequence set forth in Table 3 or otherwise known in the art, or a corresponding fragment thereof. Examples of UL30 polypeptides known in the art include UL30 polypeptides encoded by known HSV strains such as, without limitation, HG52, G, 333, and MS strains. In some embodiments, a UL30 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 49, 50 and/or 51.

In some embodiments, an HSV T cell antigen is or includes a UL52 polypeptide or fragment thereof. In various embodiments, a UL52 polypeptide or fragment thereof has at least 80% sequence identity with a UL52 amino acid sequence set forth in Table 3 or otherwise known in the art, or a corresponding fragment thereof. In some embodiments, a UL52 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with a UL1 amino acid sequence set forth in Table 3 or otherwise known in the art, or a corresponding fragment thereof. Examples of UL52 polypeptides known in the art include UL52 polypeptides encoded by known HSV strains such as, without limitation, HG52, G, 333, and MS strains. In some embodiments, a UL52 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 52, 53 and/or 54.

The US1 open reading frame encodes HSV gL (also referred to herein as UL1 polypeptide). In some embodiments, an HSV T cell antigen is or includes a US1 polypeptide or fragment thereof. In various embodiments, a US1 polypeptide or fragment thereof has at least 80% sequence identity with a US1 amino acid sequence set forth in Table 3 or otherwise known in the art, or a corresponding fragment thereof. In some embodiments, a Us1 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with a UL1 amino acid sequence set forth in Table 3 or otherwise known in the art, or a corresponding fragment thereof. Examples of US1 polypeptides known in the art include US1 polypeptides encoded by known HSV strains such as, without limitation, HG52, G, 333, and MS strains. In some embodiments, a US1 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 58, 59, 60 and/or 61.

The US7 open reading frame encodes HSV gI (also referred to herein as US7 polypeptide). In some embodiments, an HSV T cell antigen is or includes a US7 polypeptide or fragment thereof. In various embodiments, a US7 polypeptide or fragment thereof has at least 80% sequence identity with a US7 amino acid sequence set forth in Table 3 or otherwise known in the art, or a corresponding fragment thereof. In some embodiments, a UL1 polypeptide or fragment thereof has at least 80%, such as at least 80%, 85%, 90%, 91%, 92%, 93%, 94%, 95%, 96%, 97%, 99%, or 100% sequence identity with a US7 amino acid sequence set forth in Table 3 or otherwise known in the art, or a corresponding fragment thereof. Examples of US7 polypeptides known in the art include US7 polypeptides encoded by known HSV strains such as, without limitation, HG52, G, 333, and MS strains. In some embodiments, a US7 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 62, 63, 64 and/or 65.

The US8 open reading frame encodes HSV gE (also referred to herein as US8 polypeptide). In some embodiments, an HSV T cell antigen is or includes a US8 polypeptide or fragment thereof. In various embodiments, a US8 polypeptide or fragment thereof has at least 80% sequence identity with a US8 amino acid sequence set forth in Table 3 or otherwise known in the art, or a corresponding fragment thereof. In some embodiments, a UL1 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with a US8 amino acid sequence set forth in Table 3 or otherwise known in the art, or a corresponding fragment thereof. Examples of US8 polypeptides known in the art include US8 polypeptides encoded by known HSV strains such as, without limitation, HG52, G, 333, and MS strains. In some embodiments, a US8 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 66, 67, 68 and/or 69.

The UL22 open reading frame encodes HSV gH (also referred to herein as UL22 polypeptide). In some embodiments, an HSV T cell antigen is or includes a UL22 polypeptide or fragment thereof. In various embodiments, a UL22 polypeptide or fragment thereof has at least 80% sequence identity with a UL22 amino acid sequence set forth in Table 3 or otherwise known in the art, or a corresponding fragment thereof. In some embodiments, a UL22 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with a UL1 amino acid sequence set forth in Table 3 or otherwise known in the art, or a corresponding fragment thereof. Examples of UL22 polypeptides known in the art include UL22 polypeptides encoded by known HSV strains such as, without limitation, HG52, G, 333, and MS strains. In some embodiments, a UL22 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 70, 71, 72 and/or 73.

In some embodiments, an HSV T cell antigen is or includes a UL54 polypeptide or fragment thereof. In various embodiments, a UL54 polypeptide or fragment thereof has at least 80% sequence identity with a UL54 amino acid sequence set forth in Table 3 or otherwise known in the art, or a corresponding fragment thereof. In some embodiments, a UL1 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with a UL54 amino acid sequence set forth in Table 3 or otherwise known in the art, or a corresponding fragment thereof. Examples of UL54 polypeptides known in the art include UL54 polypeptides encoded by known HSV strains such as, without limitation, HG52, G, 333, and MS strains. In some embodiments, a UL54 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 55, 56, and/or 57.

In some embodiments, an HSV antigen for use in accordance with the present disclosure comprises an RL2 polypeptide or antigenic fragment thereof, an RS1 polypeptide or antigenic fragment thereof, a UL54 polypeptide or antigenic fragment thereof, a UL29 polypeptide or antigenic fragment thereof, a UL39 polypeptide or antigenic fragment thereof, a UL49 polypeptide or antigenic fragment thereof, a UL9 polypeptide or antigenic fragment thereof, a UL30 polypeptide or antigenic fragment thereof, a UL40 polypeptide or antigenic fragment thereof, a UL5 polypeptide or antigenic fragment thereof, a UL52 polypeptide or antigenic fragment thereof, a UL1 polypeptide or antigenic fragment thereof, a UL19 polypeptide or antigenic fragment thereof, a UL21 polypeptide or antigenic fragment thereof, a UL27 polypeptide or antigenic fragment thereof, a UL46 polypeptide or antigenic fragment thereof, a UL47 polypeptide or antigenic fragment thereof, a UL48 polypeptide or antigenic fragment thereof, a UL25 polypeptide or antigenic fragment thereof, or a combination thereof.

In some embodiments, a polyribonucleotide provided herein encodes one or more of an RL2 polypeptide or antigenic fragment thereof, an RS1 polypeptide or antigenic fragment thereof, a UL54 polypeptide or antigenic fragment thereof, a UL29 polypeptide or antigenic fragment thereof, a UL39 polypeptide or antigenic fragment thereof, a UL49 polypeptide or antigenic fragment thereof, a UL9 polypeptide or antigenic fragment thereof, a UL30 polypeptide or antigenic fragment thereof, a UL40 polypeptide or antigenic fragment thereof, a UL5 polypeptide or antigenic fragment thereof, a UL52 polypeptide or antigenic fragment thereof, a UL1 polypeptide or antigenic fragment thereof, a UL19 polypeptide or antigenic fragment thereof, a UL21 polypeptide or antigenic fragment thereof, a UL27 polypeptide or antigenic fragment thereof, a UL46 polypeptide or antigenic fragment thereof, a UL47 polypeptide or antigenic fragment thereof, a UL48 polypeptide or antigenic fragment thereof, a UL25 polypeptide or antigenic fragment thereof, a US1 polypeptide or antigenic fragment thereof, US7 polypeptide or antigenic fragment thereof, US8 polypeptide or antigenic fragment thereof, and UL22 polypeptide or antigenic fragment thereof.

In some embodiments, a polyribonucleotide provided herein encodes one or more of HSV (e.g., HSV-1 and/or HSV-2) antigens, wherein at least one antigen is an RL2 polypeptide or antigenic fragment thereof. In some embodiments, an RL2 polypeptide or antigenic fragment thereof comprises or consists of an amino acid sequence of CTDEIAPPLRCQSFPCLHPFCIPCMKTWIPLRNTCPLCNTPVAYLIVGVTASGSFSTIPI VNDPRTRVEAEAAVRAGTAVDFIWTGNPRTAPRSLS (SEQ ID NO: 174). In some embodiments, an RL2 polypeptide or antigenic fragment thereof comprises or consists of an amino acid sequence of

(SEQ ID NO: 175)
LPIAGVSSVVALAPYVNKTVTGDCLPVLDMETGHIGAYVVLVDQTGNVAD
LLRAAAPAWSRRTLLPEHARNCVRPPDYPTPPASEWNSLWMTPVGNMLFD
QGTLVG.

In some embodiments, a polyribonucleotide provided herein encodes one or more of HSV (e.g., HSV-1 and/or HSV-2) antigens, wherein at least one antigen is an RS1 polypeptide or antigenic fragment thereof. In some embodiments, an RS1 polypeptide or antigenic fragment thereof comprises or consists of an amino acid sequence of

(SEQ ID NO: 176)
RAAAWMRQVPDPEDVRVVILYSPLPGEDLAAGRAGGGPPPEWSAERGGLS
CLLAALGNRLCGPATAAWAGNWTGAPDVSALGAQGVLLLSTRDLAFAGAV
EFLGLLAGACDRRLIVVNAVRAADWPADGPVVSRQHAYLACEVLPAVQCA
VRWPAARDLRRTVLASGRVFGPGVFARVEAAHARLYPDAPPLRLCRGANV
RYRVRTRFGPDTLVPMSPREYRRAVLPALDGRAAAS.

In some embodiments, a polyribonucleotide provided herein encodes one or more of HSV (e.g., HSV-1 and/or HSV-2) antigens, wherein at least one antigen is a UL54 polypeptide or antigenic fragment thereof. In some embodiments, a UL54 polypeptide or antigenic fragment thereof comprises or consists of an amino acid sequence of

(SEQ ID NO: 177)
ETLVAHGPSLYRTFAANPRAASTAKAMRDCVLRQENLIEALASADETLAW
CKMCIHHNLPLRPQDPIIGTAAAVLENLATRLRPFLQCYLKARGLCGLDD
LCSRRRLSDIKDIASFVLVILARLANRVERGVSEIDYTTVGVGAGETMHF
YIPGACMAGLIEILDTHRQECSSRVCELTASHTIAPLYVHGKYFYCNSL
F.

In some embodiments, a polyribonucleotide provided herein encodes one or more of HSV (e.g., HSV-1 and/or HSV-2) antigens, wherein at least one antigen is a UL29 polypeptide or antigenic fragment thereof. In some embodiments, a UL29 polypeptide or antigenic fragment thereof comprises or consists of an amino acid sequence of

(SEQ ID NO: 178)
REDIETIAFIKRFSLDYGAINFINLAPNNVSELAMYYMANQILRYCDHST
YFINTLTAVIAGSRRPPSVQAAAAWAPQGGAGLEAGARALMDSLDAHPGA
WTSMFASCNLLRPVMAARPMVVLGLSISKYYGMAGNDRVFQAGNWASLLG
GKNACPLLIFDRTRKFVL.

In some embodiments, a polyribonucleotide provided herein encodes one or more of HSV (e.g., HSV-1 and/or HSV-2) antigens, wherein at least one antigen is a UL39 polypeptide or antigenic fragment thereof. In some embodiments, a UL39 polypeptide or antigenic fragment thereof comprises or consists of an amino acid sequence of

(SEQ ID NO: 179)
RTFGSAPRLTEDDFGLLNYALAEMRRLCLDLPPVPPNAYTPYHLREYATR
LVNGFKPLVRRSARLYRILGVLVHLRIRTREASFEEWMRSKEVDLDFGLT
ERLREHEAQLMILAQALNPYDCLIHSTPNTLVERGLQSALKYEEFYLKRF
GGHYMESVFQMYTRIAGFLA.

In some embodiments, a polyribonucleotide provided herein encodes one or more of HSV (e.g., HSV-1 and/or HSV-2) antigens, wherein at least one antigen is a UL49 polypeptide or antigenic fragment thereof. In some embodiments, a UL49 polypeptide or antigenic fragment thereof comprises or consists of an amino acid sequence of

(SEQ ID NO: 180)
KMTRGAPKASATPATDPARGRRPAQADSAVLLDAPAPTASGRTKTPAQGL
AKKLHFSTAPPSPTAPWTPRVAGENKRVFCAAVG.

In some embodiments, a polyribonucleotide provided herein encodes one or more of HSV (e.g., HSV-1 and/or HSV-2) antigens, wherein at least one antigen is a UL9 polypeptide or antigenic fragment thereof. In some embodiments, a UL9 polypeptide or antigenic fragment thereof comprises or consists of an amino acid sequence of

(SEQ ID NO: 181)
LLNNYDVLVLDEVMSTLGQLYSPTMQQLGRVDALMLRLLRTCPRIIAMDA
TANAQLVDFLCSLRGEKNVHVVIGEYAMPGFSARRCLFLPRLGPEVLQAA
LRRRGPAGGAPPPDAPPDATFFGELEARLAGGDNVCIFSSTVSFAEVVAR
FCRQFTDRVLLLHSLTPPGDVTTWGRYRVVIYTTVVTVGLSFDPPHFDSM
FAYVKPMNYGPDMVSVYQSLGRVRTLRKGELLIYMDGSGARSEPV.

In some embodiments, a polyribonucleotide provided herein encodes one or more of HSV (e.g., HSV-1 and/or HSV-2) antigens, wherein at least one antigen is a UL30 polypeptide or antigenic fragment thereof. In some embodiments, a UL30 polypeptide or antigenic fragment thereof comprises or consists of an amino acid sequence of ISCLLYDLSTTALEHILLFSLGSCDLPESHLSDLASRGLPAPVVLEFDSEFEMLLAFMT FVKQYGPEFVTGYNIINFDWPFVLTKLTEIYKVPLDGYGRMNGRGVFRVWDIGQSH F (SEQ ID NO: 182). In some embodiments, a UL30 polypeptide or antigenic fragment thereof comprises an amino acid sequence of

(SEQ ID NO: 183)
GLLPCLHVAATVTTIGREMLLATRAYVHARWAEFDQLLADFPEAAGMRAP
GPYSM.

In some embodiments, a polyribonucleotide provided herein encodes one or more of HSV (e.g., HSV-1 and/or HSV-2) antigens, wherein at least one antigen is a UL40 polypeptide or antigenic fragment thereof. In some embodiments, a UL40 polypeptide or antigenic fragment thereof comprises or consists of an amino acid sequence of

(SEQ ID NO: 184)
TSQCPDINHLRSLSILNRWLETELVFVGDEEDVSKLSEGELGFYR
FLFAFLSAADDLVTENLGGLSGLFEQKDILHYYVEQECIEVVHSR
VYNIIQLVLFHNNDQARRAYVARTINHPAIRVKVDWLEARVRECD
SIPEKFILMILIEGVFFAASFAAIAYLRTNNLLR.

In some embodiments, a polyribonucleotide provided herein encodes one or more of HSV (e.g., HSV-1 and/or HSV-2) antigens, wherein at least one antigen is a UL5 polypeptide or antigenic fragment thereof. In some embodiments, a UL5 polypeptide or antigenic fragment thereof comprises or consists of an amino acid sequence of HEFGNLMKVLEYGLPITEEHMQFVDRFVVPESYITNPANLPGWTRLFSSHKEVSAY MAKLHAYLKVTREGEFVVFTLPVLTFVSVKEFDEYRRL (SEQ ID NO: 185). In some embodiments, a UL5 polypeptide or antigenic fragment thereof comprises or consists of an amino acid sequence of

(SEQ ID NO: 186)
ELFGEVFESAPFSTYVDNVIFRGCELLTGSPRGGLMSVALQTDNY
TLMGYTYTRVFAFAEELRRRHATAGVAEFLEESPLPYIVLRDQHG
FMSVVNTNI.

In some embodiments, a polyribonucleotide provided herein encodes one or more of HSV (e.g., HSV-1 and/or HSV-2) antigens, wherein at least one antigen is a UL52 polypeptide or antigenic fragment thereof. In some embodiments, a UL52 polypeptide or antigenic fragment thereof comprises or consists of an amino acid sequence of

(SEQ ID NO: 187)
SVAAPVEVTALYATDGCVITSSLALLTNCLLGAEPLYIFSYDAYR
SDAPNGPTGAPTEQERFEGSRALYRDAGGLNGDSFRVTFCLLGTE
VGVTHHPKGRTRPMFVCRFERADDVAVLQDALGRGTPLLPAHVTA
TLDLEATFALHANIIMALTVAIVHNAPARIGSGSTAPLYEPGESM
RSVV.

In some embodiments, a polyribonucleotide provided herein encodes one or more of HSV (e.g., HSV-1 and/or HSV-2) antigens, wherein at least one antigen is a UL1 polypeptide or antigenic fragment thereof. In some embodiments, a UL1 polypeptide or antigenic fragment thereof comprises or consists of an amino acid sequence of

(SEQ ID NO: 188)
RTPADDVSWRYEAPSVIDYARIDGIFLRYHCPGLDTFLWDRHAQR
AYLVNPFLFAAGFLEDLSHSVFPADTQETT.

In some embodiments, a polyribonucleotide provided herein encodes one or more of HSV (e.g., HSV-1 and/or HSV-2) antigens, wherein at least one antigen is a UL19 polypeptide or antigenic fragment thereof. In some embodiments, a UL19 polypeptide or antigenic fragment thereof comprises or consists of an amino acid sequence of

(SEQ ID NO: 189)
DGRLLHNTQARAADAADDRPHRPADWTVHHKIYYYVLVPAFSRGR
CCTAGVREDRVYATLQNMVVPEIAPGEECPSDPVTDPAHPLHPAN
LVANTVKRMFHN.

In some embodiments, a polyribonucleotide provided herein encodes one or more of HSV (e.g., HSV-1 and/or HSV-2) antigens, wherein at least one antigen is a UL21 polypeptide or antigenic fragment thereof. In some embodiments, a UL21 polypeptide or antigenic fragment thereof comprises or consists of an amino acid sequence of

(SEQ ID NO: 190)
SPTQKLAVYYYLIHRERRMSPFPALVRLVGRYIQRHGLYVPAPDE
PTLADAMNGL.

In some embodiments, a polyribonucleotide provided herein encodes one or more of HSV (e.g., HSV-1 and/or HSV-2) antigens, wherein at least one antigen is a UL27 polypeptide or antigenic fragment thereof. In some embodiments, a UL27 polypeptide or antigenic fragment thereof comprises or consists of an amino acid sequence of NYTEGIAVVFKENIAPYKFKATMYYKDVTVSQVWFGHRYSQFMGIFEDRAPVPFEE V (SEQ ID NO: 191). In some embodiments, a UL27 polypeptide or antigenic fragment thereof comprises or consists of an amino acid sequence of

(SEQ ID NO: 192)
SVYPYDEFVLATGDFVYMSPFYGYREGSH.

In some embodiments, a polyribonucleotide provided herein encodes one or more of HSV (e.g., HSV-1 and/or HSV-2) antigens, wherein at least one antigen is a UL46 polypeptide or antigenic fragment thereof. In some embodiments, a UL46 polypeptide or antigenic fragment thereof comprises or consists of an amino acid sequence of

(SEQ ID NO: 193)
GLASDPHYDYIRHYASAAKQALGEVELSGGQLSRAILAQYWKYLQ
TVVPSGLDIPDDPAGDCDPSLHVLLRPTLLPKLLVRAPFKSGAAA
AKYAAAVAGLRDAAHRLQQYMFFMRPADPSRPSTDTALRLSELLA
YVSVLYHWASWMLWTADKYV.

In some embodiments, a polyribonucleotide provided herein encodes one or more of HSV (e.g., HSV-1 and/or HSV-2) antigens, wherein at least one antigen is a UL47 polypeptide or antigenic fragment thereof. In some embodiments, a UL47 polypeptide or antigenic fragment thereof comprises or consists of an amino acid sequence of

(SEQ ID NO: 194)
GPDAAVFRSSLGSLLYWPGVRALLGRDCRVAARYAGRMTYIATGA
LLARFNPGAVKCVLPREAAFAGRVL.

In some embodiments, a polyribonucleotide provided herein encodes one or more of HSV (e.g., HSV-1 and/or HSV-2) antigens, wherein at least one antigen is a UL48 polypeptide or antigenic fragment thereof. In some embodiments, a UL48 polypeptide or antigenic fragment thereof comprises or consists of an amino acid sequence of

(SEQ ID NO: 195)
ALFNRLLDDLGFSAGPALCTMLDTWNEDLFSGFPTNADMYRECKF
LSTLPSDVIDWGDAHVPERSPIDIRAHGDVAFPTLPATRDELPSY
YEAMAQFFRGELRA.

In some embodiments, a polyribonucleotide provided herein encodes one or more of HSV (e.g., HSV-1 and/or HSV-2) antigens, wherein at least one antigen is a UL25 polypeptide or antigenic fragment thereof. In some embodiments, a UL25 polypeptide or antigenic fragment thereof comprises or consists of an amino acid sequence of

(SEQ ID NO: 196)
FLWEDQTLLRATANTITALAVLRRLLANGNVYADRLDNRLQLGML
IPGAVPAEAIARGASGLDSGAIKSGDNNLEALCVNYVLPLYQADP
TVELTQLFPGLAALCL.

In some embodiments, an HSV (e.g., HSV-1 and/or HSV-2) antigen for use in accordance with the present disclosure is an intermediate early protein or an antigenic fragment thereof. In some embodiments, a polyribonucleotide provided herein encodes one or more HSV (e.g., HSV-1 and/or HSV-2) antigens, wherein at least one HSV antigen comprises an intermediate early protein or an antigenic fragment thereof. In some embodiments, a polyribonucleotide provided herein encodes one or more HSV (e.g., HSV-1 and/or HSV-2) antigens, wherein each of the HSV antigens comprises an intermediate early protein or an antigenic fragment thereof.

In some embodiments, an HSV-2 antigen for use in accordance with the present disclosure is an intermediate early protein or an antigenic fragment thereof. In some embodiments, a polyribonucleotide provided herein encodes one or more HSV-2 antigens, wherein at least one HSV-2 antigen comprises an intermediate early protein or an antigenic fragment thereof. In some embodiments, a polyribonucleotide provided herein encodes one or more HSV-2 antigens, wherein each of the HSV-2 antigens comprises an intermediate early protein or an antigenic fragment thereof.

In some embodiments, an HSV-2 antigen for use in accordance with the present disclosure comprises an RL2 polypeptide or antigenic fragment thereof, an RS1 polypeptide or antigenic fragment thereof, a UL54 polypeptide or antigenic fragment thereof, or a combination thereof.

In some embodiments, a polyribonucleotide provided herein encodes one or more of an RL2 polypeptide or antigenic fragment thereof, an RS1 protein or antigenic fragment thereof, and a UL54 protein or antigenic fragment thereof.

In some embodiments, an HSV (e.g., HSV-1 and/or HSV-2) antigen for use in accordance with the present disclosure is an early protein or an antigenic fragment thereof. In some embodiments, a polyribonucleotide provided herein encodes one or more HSV (e.g., HSV-1 and/or HSV-2) antigens, wherein at least one HSV antigen comprises an early protein or an antigenic fragment thereof. In some embodiments, a polyribonucleotide provided herein encodes one or more HSV (e.g., HSV-1 and/or HSV-2) antigens, wherein each of the HSV antigens comprises an early protein or an antigenic fragment thereof.

In some embodiments, an HSV-2 antigen for use in accordance with the present disclosure is an early protein or an antigenic fragment thereof. In some embodiments, a polyribonucleotide provided herein encodes one or more HSV-2 antigens, wherein at least one HSV-2 antigen comprises an early protein or an antigenic fragment thereof. In some embodiments, a polyribonucleotide provided herein encodes one or more HSV-2 antigens, wherein each of the HSV-2 antigens comprises an early protein or an antigenic fragment thereof.

In some embodiments, an HSV-2 antigen for use in accordance with the present disclosure comprises a UL29 polypeptide or antigenic fragment thereof, a UL39 polypeptide or antigenic fragment thereof, a UL49 polypeptide or antigenic fragment thereof, a UL9 polypeptide or antigenic fragment thereof, a UL30 polypeptide or antigenic fragment thereof, a UL40 polypeptide or antigenic fragment thereof, a UL5 polypeptide or antigenic fragment thereof, a UL52 polypeptide or antigenic fragment thereof, or a combination thereof.

In some embodiments, a polyribonucleotide provided herein encodes one or more of a UL29 polypeptide or antigenic fragment thereof, a UL39 polypeptide or antigenic fragment thereof, a UL49 polypeptide or antigenic fragment thereof, a UL9 polypeptide or antigenic fragment thereof, a UL30 polypeptide or antigenic fragment thereof, a UL40 polypeptide or antigenic fragment thereof, a UL5 polypeptide or antigenic fragment thereof, and a UL52 polypeptide or antigenic fragment thereof.

In some embodiments, an HSV-2 antigen for use in accordance with the present disclosure comprises a UL29 polypeptide or antigenic fragment thereof, a UL39 polypeptide or antigenic fragment thereof, a UL49 polypeptide or antigenic fragment thereof, a UL9 polypeptide or antigenic fragment thereof, or a combination thereof.

In some embodiments, a UL29 polypeptide or antigenic fragment thereof, a UL39 polypeptide or antigenic fragment thereof, a UL49 polypeptide or antigenic fragment thereof, and a UL9 polypeptide or antigenic fragment thereof.

In some embodiments, an HSV-2 antigen for use in accordance with the present disclosure comprises a UL30 polypeptide or antigenic fragment thereof, a UL40 polypeptide or antigenic fragment thereof, a UL5 polypeptide or antigenic fragment thereof, a UL52 polypeptide or antigenic fragment thereof, or a combination thereof.

In some embodiments, a polyribonucleotide provided herein encodes one or more of a UL30 polypeptide or antigenic fragment thereof, a UL40 polypeptide or antigenic fragment thereof, a UL5 polypeptide or antigenic fragment thereof, and a UL52 polypeptide or antigenic fragment thereof.

In some embodiments, an HSV (e.g., HSV-1 and/or HSV-2) antigen for use in accordance with the present disclosure is a late protein or an antigenic fragment thereof. In some embodiments, a polyribonucleotide provided herein encodes one or more HSV (e.g., HSV-1 and/or HSV-2) antigens, wherein at least one HSV antigen comprises a late protein or an antigenic fragment thereof. In some embodiments, a polyribonucleotide provided herein encodes one or more HSV (e.g., HSV-1 and/or HSV-2) antigens, wherein each of the HSV antigens comprises a late protein or an antigenic fragment thereof.

In some embodiments, an HSV-2 antigen for use in accordance with the present disclosure is a late protein or an antigenic fragment thereof. In some embodiments, a polyribonucleotide provided herein encodes one or more HSV-2 antigens, wherein at least one HSV-2 antigen comprises a late protein or an antigenic fragment thereof. In some embodiments, a polyribonucleotide provided herein encodes one or more HSV-2 antigens, wherein each of the HSV-2 antigens comprises a late protein or an antigenic fragment thereof.

In some embodiments, an HSV-2 antigen for use in accordance with the present disclosure comprises a UL1 polypeptide or antigenic fragment thereof, a UL19 polypeptide or antigenic fragment thereof, a UL21 polypeptide or antigenic fragment thereof, a UL27 polypeptide or antigenic fragment thereof, a UL46 polypeptide or antigenic fragment thereof, a UL47 polypeptide or antigenic fragment thereof, a UL48 polypeptide or antigenic fragment thereof, a UL25 polypeptide or antigenic fragment thereof, or a combination thereof.

In some embodiments, a polyribonucleotide provided herein encodes one or more of a UL1 polypeptide or antigenic fragment thereof, a UL19 polypeptide or antigenic fragment thereof, a UL21 polypeptide or antigenic fragment thereof, a UL27 polypeptide or antigenic fragment thereof, a UL46 polypeptide or antigenic fragment thereof, a UL47 polypeptide or antigenic fragment thereof, a UL48 polypeptide or antigenic fragment thereof, and a UL25 polypeptide or antigenic fragment thereof.

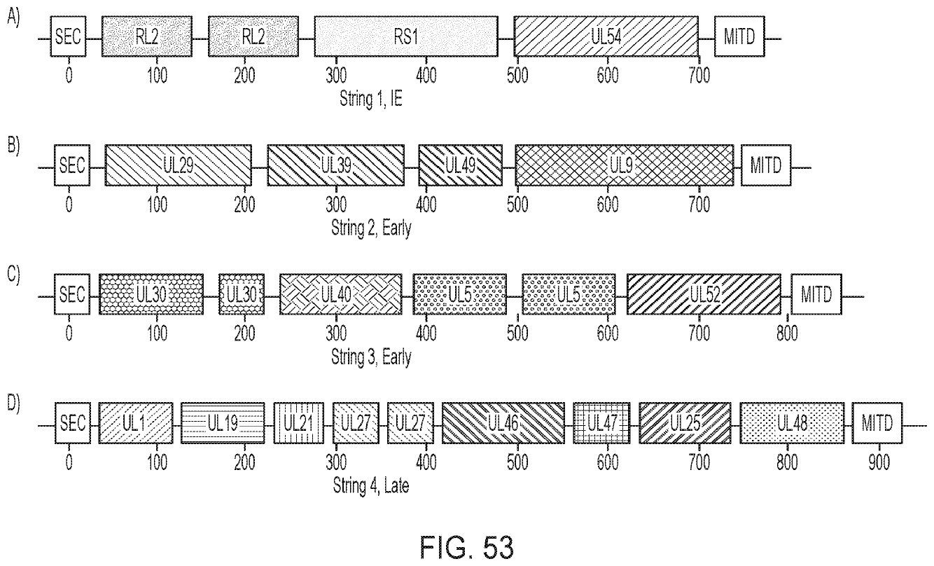

Select exemplary payloads in accordance with this disclosure encode polypeptides illustrated in FIG. 53. For example, in some embodiments, a polyribonucleotide provide herein can include, in 5′ to 3′ order, nucleotide sequences that encode an HSV-1 gD secretory signal, an RL2 polypeptide or antigenic fragment thereof, a linker, an RL2 polypeptide or antigenic fragment thereof, a linker, an RS1 polypeptide or antigenic fragment thereof, a linker, a UL54 polypeptide or fragment thereof, a linker, and a MITD (see FIG. 53A). In some embodiments, such a polyribonucleotide encodes a polypeptide having an amino acid sequence comprising or consisting of

(SEQ ID NO: 197)
MGGAAARLGAVILFVVIVGLHGVRGCTDEIAPPLRCQSFPCLHPF
CIPCMKTWIPLRNTCPLCNTPVAYLIVGVTASGSFSTIPIVNDPR
TRVEAEAAVRAGTAVDFIWTGNPRTAPRSLSGGSGGGGSGGLPIA
GVSSVVALAPYVNKTVTGDCLPVLDMETGHIGAYVVLVDQTGNVA
DLLRAAAPAWSRRTLLPEHARNCVRPPDYPTPPASEWNSLWMTPV
GNMLFDQGTLVGGGSGGGGSGGRAAAWMRQVPDPEDVRVVILYSP
LPGEDLAAGRAGGGPPPEWSAERGGLSCLLAALGNRLCGPATAAW
AGNWTGAPDVSALGAQGVLLLSTRDLAFAGAVEFLGLLAGACDRR
LIVVNAVRAADWPADGPVVSRQHAYLACEVLPAVQCAVRWPAARD
LRRTVLASGRVFGPGVFARVEAAHARLYPDAPPLRLCRGANVRYR
VRTRFGPDTLVPMSPREYRRAVLPALDGRAAASGGSGGGGSGGET
LVAHGPSLYRTFAANPRAASTAKAMRDCVLRQENLIEALASADET
LAWCKMCIHHNLPLRPQDPIIGTAAAVLENLATRLRPFLQCYLKA
RGLCGLDDLCSRRRLSDIKDIASFVLVILARLANRVERGVSEIDY
TTVGVGAGETMHFYIPGACMAGLIEILDTHRQECSSRVCELTASH
TIAPLYVHGKYFYCNSLFGGSGGGGSGGIVGIVAGLAVLAVVVIG
AVVATVMCRRKSSGGKGGSYSQAASSDSAQGSDVSLTA.

In some embodiments, a polyribonucleotide provide herein can include, in 5′ to 3′ order, nucleotide sequences that encode an HSV-1 gD secretory signal, an UL29 polypeptide or antigenic fragment thereof, a linker, an UL39 polypeptide or antigenic fragment thereof, a linker, an UL49 polypeptide or antigenic fragment thereof, a linker, a UL9 polypeptide or fragment thereof, a linker, and a MITD (see FIG. 53B). In some embodiments, such a polyribonucleotide encodes a polypeptide having an amino acid sequence comprising or consisting of

(SEQ ID NO: 198)
MGGAAARLGAVILFVVIVGLHGVRGREDIETIAFIKRFSLDYGAI
NFINLAPNNVSELAMYYMANQILRYCDHSTYFINTLTAVIAGSRR
PPSVQAAAAWAPQGGAGLEAGARALMDSLDAHPGAWTSMFASCNL
LRPVMAARPMVVLGLSISKYYGMAGNDRVFQAGNWASLLGGKNAC
PLLIFDRTRKFVLGGSGGGGSGGRTFGSAPRLTEDDFGLLNYALA
EMRRLCLDLPPVPPNAYTPYHLREYATRLVNGFKPLVRRSARLYR
ILGVLVHLRIRTREASFEEWMRSKEVDLDFGLTERLREHEAQLMI
LAQALNPYDCLIHSTPNTLVERGLQSALKYEEFYLKRFGGHYMES
VFQMYTRIAGFLAGGSGGGGSGGKMTRGAPKASATPATDPARGRR
PAQADSAVLLDAPAPTASGRTKTPAQGLAKKLHFSTAPPSPTAPW
TPRVAGFNKRVFCAAVGGGSGGGGSGGLLNNYDVLVLDEVMSTLG
QLYSPTMQQLGRVDALMLRLLRTCPRIIAMDATANAQLVDFLCSL
RGEKNVHVVIGEYAMPGFSARRCLFLPRLGPEVLQAALRRRGPAG
GAPPPDAPPDATFFGELEARLAGGDNVCIFSSTVSFAEVVARFCR
QFTDRVLLLHSLTPPGDVTTWGRYRVVIYTTVVTVGLSFDPPHFD
SMFAYVKPMNYGPDMVSVYQSLGRVRTLRKGELLIYMDGSGARSE
PVGGSGGGGSGGIVGIVAGLAVLAVVVIGAVVATVMCRRKSSGGK
GGSYSQAASSDSAQGSDVSLTA.

In some embodiments, a polyribonucleotide provide herein can include, in 5′ to 3′ order, nucleotide sequences that encode an HSV-1 gD secretory signal, an UL30 polypeptide or antigenic fragment thereof, a linker, an UL30 polypeptide or antigenic fragment thereof, a linker, an UL40 polypeptide or antigenic fragment thereof, a linker, a UL5 polypeptide or fragment thereof, a linker, a UL5 polypeptide or fragment thereof, a linker, a UL52 polypeptide or fragment thereof, a linker, and a MITD (see FIG. 53C). In some embodiments, such a polyribonucleotide encodes a polypeptide having an amino acid sequence comprising or consisting of

(SEQ ID NO: 199)
MGGAAARLGAVILFVVIVGLHGVRGISCLLYDLSTTALEHILLFS
LGSCDLPESHLSDLASRGLPAPVVLEFDSEFEMLLAFMTFVKQYG
PEFVTGYNIINFDWPFVLTKLTEIYKVPLDGYGRMNGRGVFRVWD
IGQSHFGGSGGGGSGGGLLPCLHVAATVTTIGREMLLATRAYVHA
RWAEFDQLLADFPEAAGMRAPGPYSMGGSGGGGSGGTSQCPDINH
LRSLSILNRWLETELVFVGDEEDVSKLSEGELGFYRFLFAFLSAA
DDLVTENLGGLSGLFEQKDILHYYVEQECIEVVHSRVYNIIQLVL
FHNNDQARRAYVARTINHPAIRVKVDWLEARVRECDSIPEKFILM
ILIEGVFFAASFAAIAYLRTNNLLRGGSGGGGSGGHEFGNLMKVL
EYGLPITEEHMQFVDRFVVPESYITNPANLPGWTRLFSSHKEVSA
YMAKLHAYLKVTREGEFVVFTLPVLTFVSVKEFDEYRRLGGSGGG
GSGGELFGEVFESAPFSTYVDNVIFRGCELLTGSPRGGLMSVALQ
TDNYTLMGYTYTRVFAFAEELRRRHATAGVAEFLEESPLPYIVLR
DQHGFMSVVNTNIGGSGGGGSGGSVAAPVEVTALYATDGCVITSS
LALLTNCLLGAEPLYIFSYDAYRSDAPNGPTGAPTEQERFEGSRA
LYRDAGGLNGDSFRVTFCLLGTEVGVTHHPKGRTRPMFVCRFERA
DDVAVLQDALGRGTPLLPAHVTATLDLEATFALHANIIMALTVAI
VHNAPARIGSGSTAPLYEPGESMRSVVGGSGGGGSGGIVGIVAGL
AVLAVVVIGAVVATVMCRRKSSGGKGGSYSQAASSDSAQGSDVSL
TA.

In some embodiments, a polyribonucleotide provide herein can include, in 5′ to 3′ order, nucleotide sequences that encode an HSV-1 gD secretory signal, an UL1 polypeptide or antigenic fragment thereof, a linker, an UL19 polypeptide or antigenic fragment thereof, a linker, an UL21 polypeptide or antigenic fragment thereof, a linker, a UL27 polypeptide or fragment thereof, a linker, a UL27 polypeptide or fragment thereof, a linker, a UL46 polypeptide or fragment thereof, a linker, a UL47 polypeptide or fragment thereof, a linker, a UL25 polypeptide or fragment thereof, a linker, a UL48 polypeptide or fragment thereof, a linker, and a MITD (see FIG. 53D). In some embodiments, such a polyribonucleotide encodes a polypeptide having an amino acid sequence comprising or consisting of

(SEQ ID NO: 200)
MGGAAARLGAVILFVVIVGLHGVRGRTPADDVSWRYEAPSVIDYA
RIDGIFLRYHCPGLDTFLWDRHAQRAYLVNPFLFAAGFLEDLSHS
VFPADTQETTGGSGGGGSGGDGRLLHNTQARAADAADDRPHRPAD
WTVHHKIYYYVLVPAFSRGRCCTAGVRFDRVYATLQNMVVPEIAP
GEECPSDPVTDPAHPLHPANLVANTVKRMFHNGGSGGGGSGGSPT
QKLAVYYYLIHRERRMSPFPALVRLVGRYIQRHGLYVPAPDEPTL
ADAMNGLGGSGGGGSGGNYTEGIAVVFKENIAPYKFKATMYYKDV
TVSQVWFGHRYSQFMGIFEDRAPVPFEEVGGSGGGGSGGSVYPYD
EFVLATGDFVYMSPFYGYREGSHGGSGGGGSGGGLASDPHYDYIR
HYASAAKQALGEVELSGGQLSRAILAQYWKYLQTVVPSGLDIPDD
PAGDCDPSLHVLLRPTLLPKLLVRAPFKSGAAAAKYAAAVAGLRD
AAHRLQQYMFFMRPADPSRPSTDTALRLSELLAYVSVLYHWASWM
LWTADKYVGGSGGGGSGGGPDAAVFRSSLGSLLYWPGVRALLGRD
CRVAARYAGRMTYIATGALLARFNPGAVKCVLPREAAFAGRVLGG
SGGGGSGGFLWEDQTLLRATANTITALAVLRRLLANGNVYADRLD
NRLQLGMLIPGAVPAEAIARGASGLDSGAIKSGDNNLEALCVNYV
LPLYQADPTVELTQLFPGLAALCLGGSGGGGSGGALFNRLLDDLG
FSAGPALCTMLDTWNEDLFSGFPTNADMYRECKFLSTLPSDVIDW
GDAHVPERSPIDIRAHGDVAFPTLPATRDELPSYYEAMAQFFRGE
LRAGGSGGGGSGGIVGIVAGLAVLAVVVIGAVVATVMCRRKSSGG
KGGSYSQAASSDSAQGSDVSLTA.

In some embodiments, a polyribonucleotide provide herein can include, in 5′ to 3′ order, nucleotide sequences that encode an HSV-1 gD secretory signal, an UL54 polypeptide or antigenic fragment thereof, a linker, an RS1 polypeptide or antigenic fragment thereof, a linker, an RL2 polypeptide or antigenic fragment thereof, a linker, a RL2 polypeptide or fragment thereof, a linker, and a MITD. In some embodiments, such a polyribonucleotide encodes a polypeptide having an amino acid sequence comprising or consisting of

(SEQ ID NO: 201)
MGGAAARLGAVILFVVIVGLHGVRGETLVAHGPSLYRTFAANPRA
ASTAKAMRDCVLRQENLIEALASADETLAWCKMCIHHNLPLRPQD
PIIGTAAAVLENLATRLRPFLQCYLKARGLCGLDDLCSRRRLSDI
KDIASFVLVILARLANRVERGVSEIDYTTVGVGAGETMHFYIPGA
CMAGLIEILDTHRQECSSRVCELTASHTIAPLYVHGKYFYCNSLF
GGSGGGGSGGRAAAWMRQVPDPEDVRVVILYSPLPGEDLAAGRAG
GGPPPEWSAERGGLSCLLAALGNRLCGPATAAWAGNWTGAPDVSA
LGAQGVLLLSTRDLAFAGAVEFLGLLAGACDRRLIVVNAVRAADW
PADGPVVSRQHAYLACEVLPAVQCAVRWPAARDLRRTVLASGRVF
GPGVFARVEAAHARLYPDAPPLRLCRGANVRYRVRTRFGPDTLVP
MSPREYRRAVLPALDGRAAASGGSGGGGSGGCTDEIAPPLRCQSF
PCLHPFCIPCMKTWIPLRNTCPLCNTPVAYLIVGVTASGSFSTIP
IVNDPRTRVEAEAAVRAGTAVDFIWTGNPRTAPRSLSGGSGGGGS
GGLPIAGVSSVVALAPYVNKTVTGDCLPVLDMETGHIGAYVVLVD
QTGNVADLLRAAAPAWSRRTLLPEHARNCVRPPDYPTPPASEWNS
LWMTPVGNMLFDQGTLVGGGSGGGGSGGIVGIVAGLAVLAVVVIG
AVVATVMCRRKSSGGKGGSYSQAASSDSAQGSDVSLTA.

In some embodiments, a polyribonucleotide provide herein can include, in 5′ to 3′ order, nucleotide sequences that encode an HSV-1 gD secretory signal, an UL9 polypeptide or antigenic fragment thereof, a linker, an UL49 polypeptide or antigenic fragment thereof, a linker, an UL39 polypeptide or antigenic fragment thereof, a linker, a UL29 polypeptide or fragment thereof, a linker, and a MITD. In some embodiments, such a polyribonucleotide encodes a polypeptide having an amino acid sequence comprising or consisting of

(SEQ ID NO: 202)
MGGAAARLGAVILFVVIVGLHGVRGLLNNYDVLVLDEVMSTLGQLYSPTM
QQLGRVDALMLRLLRTCPRIIAMDATANAQLVDFLCSLRGEKNVHVVIGE
YAMPGFSARRCLFLPRLGPEVLQAALRRRGPAGGAPPPDAPPDATFFGEL
EARLAGGDNVCIFSSTVSFAEVVARFCROFTDRVLLLHSLTPPGDVTTWG
RYRVVIYTTVVTVGLSFDPPHFDSMFAYVKPMNYGPDMVSVYQSLGRVRT
LRKGELLIYMDGSGARSEPVGGSGGGGSGGKMTRGAPKASATPATDPARG
RRPAQADSAVLLDAPAPTASGRTKTPAQGLAKKLHFSTAPPSPTAPWTPR
VAGENKRVFCAAVGGGSGGGGSGGRTFGSAPRLTEDDFGLLNYALAEMRR
LCLDLPPVPPNAYTPYHLREYATRLVNGFKPLVRRSARLYRILGVLVHLR
IRTREASFEEWMRSKEVDLDFGLTERLREHEAQLMILAQALNPYDCLIHS
TPNTLVERGLQSALKYEEFYLKRFGGHYMESVFQMYTRIAGFLAGGSGGG
GSGGREDIETIAFIKRFSLDYGAINFINLAPNNVSELAMYYMANQILRYC
DHSTYFINTLTAVIAGSRRPPSVQAAAAWAPQGGAGLEAGARALMDSLDA
HPGAWTSMFASCNLLRPVMAARPMVVLGLSISKYYGMAGNDRVFQAGNWA
SLLGGKNACPLLIFDRTRKFVLGGSGGGGSGGIVGIVAGLAVLAVVVIGA
VVATVMCRRKSSGGKGGSYSQAASSDSAQGSDVSLTA.

In some embodiments, a polyribonucleotide provide herein can include, in 5′ to 3′ order, nucleotide sequences that encode an HSV-1 gD secretory signal, an UL52 polypeptide or antigenic fragment thereof, a linker, an UL5 polypeptide or antigenic fragment thereof, a linker, an UL5 polypeptide or antigenic fragment thereof, a linker, a UL40 polypeptide or fragment thereof, a linker, a UL30 polypeptide or fragment thereof, a linker, a UL30 polypeptide or fragment thereof, a linker, and a MITD. In some embodiments, such a polyribonucleotide encodes a polypeptide having an amino acid sequence comprising or consisting of

(SEQ ID NO: 203)
MGGAAARLGAVILFVVIVGLHGVRGSVAAPVEVTALYATDGCVITSSLAL
LTNCLLGAEPLYIFSYDAYRSDAPNGPTGAPTEQERFEGSRALYRDAGGL
NGDSFRVTFCLLGTEVGVTHHPKGRTRPMFVCRFERADDVAVLQDALGRG
TPLLPAHVTATLDLEATFALHANIIMALTVAIVHNAPARIGSGSTAPLYE
PGESMRSVVGGSGGGGSGGHEFGNLMKVLEYGLPITEEHMQFVDRFVVPE
SYITNPANLPGWTRLFSSHKEVSAYMAKLHAYLKVTREGEFVVFTLPVLT
FVSVKEFDEYRRLGGSGGGGSGGELFGEVFESAPFSTYVDNVIFRGCELL
TGSPRGGLMSVALQTDNYTLMGYTYTRVFAFAEELRRRHATAGVAEFLEE
SPLPYIVLRDQHGFMSVVNTNIGGSGGGGSGGTSQCPDINHLRSLSILNR
WLETELVFVGDEEDVSKLSEGELGFYRFLFAFLSAADDLVTENLGGLSGL
FEQKDILHYYVEQECIEVVHSRVYNIIQLVLFHNNDQARRAYVARTINHP
AIRVKVDWLEARVRECDSIPEKFILMILIEGVFFAASFAAIAYLRTNNLL
RGGSGGGGSGGISCLLYDLSTTALEHILLFSLGSCDLPESHLSDLASRGL
PAPVVLEFDSEFEMLLAFMTFVKQYGPEFVTGYNIINFDWPFVLTKLTEI
YKVPLDGYGRMNGRGVFRVWDIGQSHFGGSGGGGSGGGLLPCLHVAATVT
TIGREMLLATRAYVHARWAEFDQLLADFPEAAGMRAPGPYSMGGSGGGGS
GGIVGIVAGLAVLAVVVIGAVVATVMCRRKSSGGKGGSYSQAASSDSAQG
SDVSLTA.

In some embodiments, a polyribonucleotide provide herein can include, in 5′ to 3′ order, nucleotide sequences that encode an HSV-1 gD secretory signal, an UL48 polypeptide or antigenic fragment thereof, a linker, an UL25 polypeptide or antigenic fragment thereof, a linker, an UL47 polypeptide or antigenic fragment thereof, a linker, a UL46 polypeptide or fragment thereof, a linker, a UL27 polypeptide or fragment thereof, a linker, a UL27 polypeptide or fragment thereof, a linker, a UL21 polypeptide or fragment thereof, a linker, a UL19 polypeptide or fragment thereof, a linker, a UL1 polypeptide or fragment thereof, a linker, and a MITD. In some embodiments, such a polyribonucleotide encodes a polypeptide having an amino acid sequence comprising or consisting of

(SEQ ID NO: 204)
MGGAAARLGAVILFVVIVGLHGVRGALFNRLLDDLGFSAGPALCTMLDTW
NEDLFSGFPTNADMYRECKFLSTLPSDVIDWGDAHVPERSPIDIRAHGDV
AFPTLPATRDELPSYYEAMAQFFRGELRAGGSGGGGSGGFLWEDQTLLRA
TANTITALAVLRRLLANGNVYADRLDNRLQLGMLIPGAVPAEAIARGASG
LDSGAIKSGDNNLEALCVNYVLPLYQADPTVELTQLFPGLAALCLGGSGG
GGSGGGPDAAVFRSSLGSLLYWPGVRALLGRDCRVAARYAGRMTYIATGA
LLARFNPGAVKCVLPREAAFAGRVLGGSGGGGSGGGLASDPHYDYIRHYA
SAAKQALGEVELSGGQLSRAILAQYWKYLQTVVPSGLDIPDDPAGDCDPS
LHVLLRPTLLPKLLVRAPFKSGAAAAKYAAAVAGLRDAAHRLQQYMFFMR
PADPSRPSTDTALRLSELLAYVSVLYHWASWMLWTADKYVGGSGGGGSGG
NYTEGIAVVFKENIAPYKFKATMYYKDVTVSQVWFGHRYSQFMGIFEDRA
PVPFEEVGGSGGGGSGGSVYPYDEFVLATGDFVYMSPFYGYREGSHGGSG
GGGSGGSPTQKLAVYYYLIHRERRMSPFPALVRLVGRYIQRHGLYVPAPD
EPTLADAMNGLGGSGGGGSGGDGRLLHNTQARAADAADDRPHRPADWTVH
HKIYYYVLVPAFSRGRCCTAGVRFDRVYATLQNMVVPEIAPGEECPSDPV
TDPAHPLHPANLVANTVKRMFHNGGSGGGGSGGRTPADDVSWRYEAPSVI
DYARIDGIFLRYHCPGLDTFLWDRHAQRAYLVNPFLFAAGFLEDLSHSVF
PADTQETTGGSGGGGSGGIVGIVAGLAVLAVVVIGAVVATVMCRRKSSGG
KGGSYSQAASSDSAQGSDVSLTA.

In some embodiments, a polyribonucleotide provide herein can include, in 5′ to 3′ order, nucleotide sequences that encode an HSV-2 gD secretory signal, an RL2 polypeptide or antigenic fragment thereof, a linker, an RL2 polypeptide or antigenic fragment thereof, a linker, an RS1 polypeptide or antigenic fragment thereof, a linker, a UL54 polypeptide or fragment thereof, a linker, and a MITD. In some embodiments, such a polyribonucleotide encodes a polypeptide having an amino acid sequence comprising or consisting of

(SEQ ID NO: 205)
MGRLTSGVGTAALLVVAVGLRVVCACTDEIAPPLRCQSFPCLHPFCIPCM
KTWIPLRNTCPLCNTPVAYLIVGVTASGSFSTIPIVNDPRTRVEAEAAVR
AGTAVDFIWTGNPRTAPRSLSGGSGGGGSGGLPIAGVSSVVALAPYVNKT
VTGDCLPVLDMETGHIGAYVVLVDQTGNVADLLRAAAPAWSRRTLLPEHA
RNCVRPPDYPTPPASEWNSLWMTPVGNMLFDQGTLVGGGSGGGGSGGRAA
AWMRQVPDPEDVRVVILYSPLPGEDLAAGRAGGGPPPEWSAERGGLSCLL
AALGNRLCGPATAAWAGNWTGAPDVSALGAQGVLLLSTRDLAFAGAVEFL
GLLAGACDRRLIVVNAVRAADWPADGPVVSRQHAYLACEVLPAVQCAVRW
PAARDLRRTVLASGRVFGPGVFARVEAAHARLYPDAPPLRLCRGANVRYR
VRTRFGPDTLVPMSPREYRRAVLPALDGRAAASGGSGGGGSGGETLVAHG
PSLYRTFAANPRAASTAKAMRDCVLRQENLIEALASADETLAWCKMCIHH
NLPLRPQDPIIGTAAAVLENLATRLRPFLQCYLKARGLCGLDDLCSRRRL
SDIKDIASFVLVILARLANRVERGVSEIDYTTVGVGAGETMHFYIPGACM
AGLIEILDTHRQECSSRVCELTASHTIAPLYVHGKYFYCNSLFGGSGGGG
SGGIVGIVAGLAVLAVVVIGAVVATVMCRRKSSGGKGGSYSQAASSDSAQ
GSDVSLTA.

In some embodiments, a polyribonucleotide provide herein can include, in 5′ to 3′ order, nucleotide sequences that encode an HSV-2 gD secretory signal, an UL54 polypeptide or antigenic fragment thereof, a linker, an RS1 polypeptide or antigenic fragment thereof, a linker, an RL2 polypeptide or antigenic fragment thereof, a linker, a RL2 polypeptide or fragment thereof, a linker, and a MITD. In some embodiments, such a polyribonucleotide encodes a polypeptide having an amino acid sequence comprising or consisting of

(SEQ ID NO: 206)
MGRLTSGVGTAALLVVAVGLRVVCAETLVAHGPSLYRTFAANPRAASTAK
AMRDCVLRQENLIEALASADETLAWCKMCIHHNLPLRPQDPIIGTAAAVL
ENLATRLRPFLQCYLKARGLCGLDDLCSRRRLSDIKDIASFVLVILARLA
NRVERGVSEIDYTTVGVGAGETMHFYIPGACMAGLIEILDTHRQECSSRV
CELTASHTIAPLYVHGKYFYCNSLFGGSGGGGSGGRAAAWMRQVPDPEDV
RVVILYSPLPGEDLAAGRAGGGPPPEWSAERGGLSCLLAALGNRLCGPAT
AAWAGNWTGAPDVSALGAQGVLLLSTRDLAFAGAVEFLGLLAGACDRRLI
VVNAVRAADWPADGPVVSRQHAYLACEVLPAVQCAVRWPAARDLRRTVLA
SGRVFGPGVFARVEAAHARLYPDAPPLRLCRGANVRYRVRTRFGPDTLVP
MSPREYRRAVLPALDGRAAASGGSGGGGSGGCTDEIAPPLRCQSFPCLHP
FCIPCMKTWIPLRNTCPLCNTPVAYLIVGVTASGSFSTIPIVNDPRTRVE
AEAAVRAGTAVDFIWTGNPRTAPRSLSGGSGGGGSGGLPIAGVSSVVALA
PYVNKTVTGDCLPVLDMETGHIGAYVVLVDQTGNVADLLRAAAPAWSRRT
LLPEHARNCVRPPDYPTPPASEWNSLWMTPVGNMLFDQGTLVGGGSGGGG
SGGIVGIVAGLAVLAVVVIGAVVATVMCRRKSSGGKGGSYSQAASSDSAQ
GSDVSLTA.

### 4. Glycoprotein Antigens and Related Constructs

Provided herein are HSV antigens that are HSV glycoproteins. In some embodiments, an HSV glycoprotein is an HSV envelope glycoprotein. In some embodiments, an HSV glycoprotein is an HSV gB, an HSV gD, an HSV gH, an HSV gL, an HSV gI, an HSV gE, or an HSV gC.

Further, provided herein are polyribonucleotides that encode an HSV glycoprotein or antigenic fragment thereof. In some embodiments, a polyribonucleotide described herein encodes an HSV glycoprotein. In some embodiments, a polyribonucleotide described herein encodes an HSV envelope glycoprotein. In some embodiments, a polyribonucleotide described herein encodes an HSV gB, an HSV gD, an HSV gH, an HSV gL, an HSV gI, an HSV gE, or an HSV gC.

In some embodiments, a polyribonucleotide according to the present disclosure encodes a polypeptide that comprises one or more HSV antigens or antigenic fragments. In some embodiments, a polypeptide comprises a single HSV antigen or antigenic fragment. In some embodiments, a polypeptide comprises a single HSV antigen. In some embodiments, an HSV antigen is a full length antigen (e.g., full length glycoprotein). In some embodiments, an HSV antigen is an HSV B cell antigen. In some embodiments, one or more HSV antigens or antigenic fragments thereof comprise one or more HSV glycoproteins. In some embodiments, one or more HSV glycoproteins comprise an HSV glycoprotein B (gB), an HSV glycoprotein E (gE), an HSV glycoprotein G (gG), an HSV glycoprotein H (gH), an HSV glycoprotein I (gI), an HSV glycoprotein L (gL), or a combination thereof.

The UL27 open reading frame encodes HSV gB. In some embodiments, an HSV antigen (e.g., a B cell antigen for HSV) is or includes an HSV gB or fragment thereof. In various embodiments, a HSV gB or fragment thereof has at least 80% sequence identity with a HSV gB amino acid sequence set forth in Table 3 or otherwise known in the art, or a corresponding fragment thereof. In some embodiments, an HSV gB polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with a HSV gB amino acid sequence set forth in Table 3 or otherwise known in the art, or a corresponding fragment thereof. Examples of HSV gB known in the art include HSV gB encoded by known HSV strains such as, without limitation, HG52, G, 333, and MS strains. In some embodiments, a HSV gB or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 7, 8, 9 and/or 74. In some embodiments, a HSV gB polypeptide or fragment thereof comprises or consists of an amino acid sequence as set forth in SEQ ID NOs: 7, 8, 9 and/or 74. In some embodiments, an HSV glycoprotein is a full-length gB glycoprotein.

The US6 open reading frame encodes HSV gD. In some embodiments, an HSV antigen (e.g., a B cell antigen for HSV) is or includes a HSV gD or fragment thereof. In various embodiments, a HSV gD or fragment thereof has at least 80% sequence identity with a HSV gD amino acid sequence set forth in Table 3 or otherwise known in the art, or a corresponding fragment thereof. In some embodiments, a HSV gD or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with a HSV gD amino acid sequence set forth in Table 3 or otherwise known in the art, or a corresponding fragment thereof. Examples of HSV gD known in the art include HSV gD encoded by known HSV strains such as, without limitation, HG52, G, 333, and MS strains. In some embodiments, an HSV gD or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NO: 221. In some embodiments, an HSV gD or fragment thereof comprises or consists of an amino acid sequence as set forth in SEQ ID NO: 221. In some embodiments, an HSV glycoprotein is a full-length gD glycoprotein.

The UL44 open reading frame encodes HSV gC. In some embodiments, an HSV antigen (e.g., a B cell antigen for HSV) is or includes a HSV gC or fragment thereof. In various embodiments, a HSV gC or fragment thereof has at least 80% sequence identity with a HSV gC amino acid sequence set forth in Table 3 or otherwise known in the art. In some embodiments, an HSV gC or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with a US6 amino acid sequence set forth in Table 3 or otherwise known in the art, or a corresponding fragment thereof. Examples of HSV gC known in the art include HSV gC encoded by known HSV strains such as, without limitation, HG52, G, 333, and MS strains. In some embodiments, an HSV gC or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NO: 222. In some embodiments, an HSV gC or fragment thereof comprises or consists of an amino acid sequence as set forth in SEQ ID NO: 222. In some embodiments, an HSV glycoprotein is a full-length gC glycoprotein.

The US1 open reading frame encodes HSV gL. In some embodiments, an HSV antigen (e.g., a B cell antigen for HSV) is or includes an HSV gL or fragment thereof. In various embodiments, an HSV gL or fragment thereof has at least 80% sequence identity with a US1 amino acid sequence set forth in Table 3 or otherwise known in the art, or a corresponding fragment thereof. In some embodiments, an HSV gL or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with a HSV gL amino acid sequence set forth in Table 3 or otherwise known in the art, or a corresponding fragment thereof. Examples of HSV gL known in the art include HSV gL encoded by known HSV strains such as, without limitation, HG52, G, 333, and MS strains. In some embodiments, HSV gL or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 58, 59, 60 and/or 61. In some embodiments, an HSV gL or fragment thereof comprises or consists of an amino acid sequence as set forth in SEQ ID NOs: 58, 59, 60 and/or 61. In some embodiments, an HSV glycoprotein is a full-length gL glycoprotein.

The US7 open reading frame encodes HSV gI. In some embodiments, an HSV antigen (e.g., a B cell antigen for HSV) is or includes an HSV gI or fragment thereof. In various embodiments, an HSV gI or fragment thereof has at least 80% sequence identity with an HSV gI amino acid sequence set forth in Table 3 or otherwise known in the art, or a corresponding fragment thereof. In some embodiments, an HSV gI or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with a HSV gL amino acid sequence set forth in Table 3 or otherwise known in the art, or a corresponding fragment thereof. Examples of HSV gI known in the art include HSV gI encoded by known HSV strains such as, without limitation, HG52, G, 333, and MS strains. In some embodiments, an HSV gI or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 62, 63, 64 and/or 65. In some embodiments, an HSV gI or fragment thereof comprises or consists of an amino acid sequence as set forth in SEQ ID NOs: 62, 63, 64 and/or 65. In some embodiments, an HSV glycoprotein is a full-length gI glycoprotein.

The US8 open reading frame encodes HSV gE. In some embodiments, an HSV antigen (e.g., a B cell antigen for HSV) is or includes an HSV gE or fragment thereof. In various embodiments, an HSV gE or fragment thereof has at least 80% sequence identity with an HSV gE amino acid sequence set forth in Table 3 or otherwise known in the art, or a corresponding fragment thereof. In some embodiments, an HSV gI or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with a HSV gE amino acid sequence set forth in Table 3 or otherwise known in the art, or a corresponding fragment thereof. Examples of HSV gE known in the art include HSV gE encoded by known HSV strains such as, without limitation, HG52, G, 333, and MS strains. In some embodiments, a US8 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 66, 67, 68 and/or 69. In some embodiments, an HSV gE or fragment thereof comprises or consists of an amino acid sequence as set forth in SEQ ID NOs: 66, 67, 68 and/or 69. In some embodiments, an HSV glycoprotein is a full-length gE glycoprotein. The UL22 open reading frame encodes HSV gH. In some embodiments, an HSV antigen (e.g., a B cell antigen for HSV) is or includes an HSV gH or fragment thereof. In various embodiments, an HSV gH or fragment thereof has at least 80% sequence identity with an HSV gH amino acid sequence set forth in Table 3 or otherwise known in the art, or a corresponding fragment thereof. In some embodiments, an HSV gH or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with a HSV gH amino acid sequence set forth in Table 3 or otherwise known in the art, or a corresponding fragment thereof. Examples of HSV gH known in the art include HSV gH encoded by known HSV strains such as, without limitation, HG52, G, 333, and MS strains. In some embodiments, a UL22 polypeptide or fragment thereof has at least 80%, such as at least 85%, at least 90%, at least 91%, at least 92%, at least 93%, at least 94%, at least 95%, at least 96%, at least 97%, at least 98%, at least 99%, or 100% sequence identity with an amino acid sequence as set forth in SEQ ID NOs: 70, 71, 72 and/or 73. In some embodiments, an HSV gH or fragment thereof comprises or consists of an amino acid sequence as set forth in SEQ ID NOs: 70, 71, 72 and/or 73. In some embodiments, an HSV glycoprotein is a full-length gH glycoprotein.

### B. Secretory Signals

In some embodiments, an HSV (e.g., HSV-1 and/or HSV-2) antigen construct described herein includes a secretory signal, e.g., that is functional in mammalian cells. In some embodiments, a utilized secretory signal is a heterologous secretory signal. In some embodiments, a heterologous secretory signal comprises or consists of a non-human secretory signal. In some embodiments, a heterologous secretory signal comprises or consists of a viral secretory signal. In some embodiments, a viral secretory signal comprises or consists of an HSV secretory signal (e.g., an HSV-1 or HSV-2 secretory signal).

In some embodiments, a secretory signal comprises or consists of an Ebola virus secretory signal. In some embodiments, an Ebola virus secretory signal comprises or consists of an Ebola virus spike glycoprotein (SGP) secretory signal.

In some embodiments, a secretory signal is characterized by a length of about 15 to 30 amino acids.

In many embodiments, a secretory signal is positioned at the N-terminus of an HSV (e.g., HSV-1 and/or HSV-2) antigen construct as described herein. In some embodiments, a secretory signal preferably allows transport of the HSV (e.g., HSV-1 and/or HSV-2) antigen construct with which it is associated into a defined cellular compartment, preferably a cell surface, endoplasmic reticulum (ER) or endosomal-lysosomal compartment.

In some embodiments, a secretory signal is selected from an S1S2 signal peptide (e.g., aa 1-19), an immunoglobulin secretory signal (e.g., aa 1-22), an HSV-1 gD signal peptide (MGGAAARLGAVILFVVIVGLHGVRSKY; SEQ ID NO: 85), an HSV-2 gD signal peptide (MGRLTSGVGTAALLVVAVGLRVVCA; SEQ ID NO: 87); a human SPARC signal peptide, a human insulin isoform 1 signal, a human albumin signal peptide, etc. Those skilled in the art will be aware of other secretory signal such as, for example, as disclosed in WO2017/081082, which is incorporated herein by reference in its entirety (e.g., SEQ ID NOs: 1-1115 and 1728, or fragments variants thereof).

In some embodiments, a HSV (e.g., HSV-1 and/or HSV-2) antigen construct described herein does not comprise a secretory signal.

In certain embodiments, a signal peptide is an IgG signal peptide, such as an IgG kappa signal peptide.

In some embodiments, an HSV secretory signal comprises or consists of an HSV glycoprotein D (gD) secretory signal.

In some embodiments, a string construct sequence encodes an antigen that may comprise or otherwise be linked to a signal sequence (e.g., secretory signal), such as those listed in Table 7 or at least a sequence having 1, 2, 3, 4, or 5 amino acid differences relative thereto. In some embodiments, a secretory signal such as MFVFLVLLPLVSSQCVNLT (SEQ ID NO: 90), or at least a sequence having 1, 2, 3, 4, or at the most 5 amino acid differences relative thereto is utilized.

In some embodiments, a secretory signal is selected from a gI signal peptide. In some embodiments, a secretory signal such as MPGRSLQGLAILGLWVCATGLVVR (SEQ ID NO: 107), or at least a sequence having 1, 2, 3, 4, or at the most 5 amino acid differences relative thereto is utilized. In some embodiments, a secretory signal such as MPGRSLQGLAILGLWVCATGL (SEQ ID NO: 108), or at least a sequence having 1, 2, 3, 4, or at the most 5 amino acid differences relative thereto is utilized.

In some embodiments, a secretory signal is one listed in Table 7 and/or Table 8, or a secretory signal having 1, 2, 3, 4, or 5 amino acid differences relative thereto. In some embodiments, a secretory signal is selected from those included in the Table 7 below and/or those encoded by the sequences in Table 8 below.

TABLE 8
Exemplary secretory signals

| | | SEQ ID |
|---|---|---|
| Secretory Signal | Sequence (Amino Acid) | NO: |
| HSV-1 gD_SKY | MGGAAARLGAVILFVVIVGLHGVRSKY | 85 |
| HSV-1 gD_RG | MGGAAARLGAVILFVVIVGLHGVRG | 86 |
| HSV-2 gD SP | MGRLTSGVGTAALLVVAVGLRVVCA | 87 |
| HSV-2 gD + KYA | MGRLTSGVGTAALLVVAVGLRVVCAKYA | 88 |
| Csp (isolate 3D7) | MMRKLAILSVSSFLFVEA | 89 |
| SARS-COV-2-S | MFVFLVLLPLVSSQCVNLT | 90 |
| human Ig heavy chain | MDWIWRILFLVGAATGAHSQM | 91 |
| secretory signal | | |
| (huSec) | | |
| HuIgGk secretory | METPAQLLFLLLLWLPDTTG | 92 |
| signal | | |
| IgE heavy chain | MDWTWILFLVAAATRVHS | 93 |
| epsilon-1 secretory | | |
| signal | | |
| Japanese encephalitis | MLGSNSGQRVVFTILLLLVAPAYS | 94 |
| PRM secretory signal | | |
| VSVg protein | MKCLLYLAFLFIGVNCA | 95 |
| secretory signal | | |
| TRIO | MCRGLSAVLILLVSLSAQLHVVVG | 96 |
| human Ig heavy chain | MELGLSWIFLLAILKGVQC | 97 |
| secretory signal 1 | | |
| human Ig heavy chain | MELGLRWVFLVAILEGVQC | 98 |
| secretory signal 2 | | |
| human Ig heavy chain | MKHLWFFLLLVAAPRWVLS | 99 |
| secretory signal 3 | | |
| human Ig heavy chain | MDWTWRILFLVAAATGAHS | 100 |
| secretory signal 4 | | |
| human Ig heavy chain | MDWTWRFLFVVAAATGVQS | 101 |
| secretory signal 5 | | |
| human Ig heavy chain | MEFGLSWLFLVAILKGVQC | 102 |
| secretory signal 6 | | |
| human Ig heavy chain | MEFGLSWVFLVALFRGVQC | 103 |
| secretory signal 7 | | |
| human Ig heavy chain | MDLLHKNMKHLWFFLLLVAAPRWVLS | 104 |
| secretory signal 8 | | |
| human Ig kappa chain | MDMRVPAQLLGLLLLWLSGARC | 105 |
| secretory signal 1 | | |
| human Ig kappa chain | MKYLLPTAAAGLLLLAAQPAMA | 106 |
| secretory signal 2 | | |
| HSV-2 gIVVR | MPGRSLQGLAILGLWVCATGLVVR | 107 |
| HSV-2 gI | MPGRSLQGLAILGLWVCATGL | 108 |
| HSV-1 gD (2) | MGGAAARLGAVILFVVIVGLHGVRGKY | 109 |
| HSV-2 gD _KYAL | MGRLTSGVGTAALLVVAVGLRVVCAKYAL | 110 |
| HSV-2 gD_KYALA | MGRLTSGVGTAALLVVAVGLRVVCAKYALA | 111 |
| HSV-2 gC signal | MALGRVGLAVGLWGLLWVGVVVVLANA | 112 |
| peptide | | |
| IL2 signal peptide | MRMQLLLLIALSLALVINS | 113 |
| HSV-1 gB | MHQGAPSWGRRWFVVWALLGLTLGVLVASAAP | 114 |
| HSV-2 gE | MARGAGLVFFVGVWVVSCLAAAPRTS | 115 |
| Ebola spike | MGVTGILQLPRDRFKRTSFFLWVIILFQRTFSIP | 116 |
| glycoprotein GP | | |
| HLA-DR signal | MAISGVPVLGFFIIAVLMSAQESWA | 117 |
| peptide | | |
| | MDSKGSSQKGSRLLLLLVVSNLLLPQGVVG | 118 |
| Japanese encephalitis | MWLVSLAIVTACAGA | 119 |
| JEV signal sequence | | |
| | MFVFLVLLPLVSSQC | 120 |
| HSV-1 gD (strain | MGGAAARLGAVILFVVIVGLHGVRGKYA | 220 |
| KOS) | | |

TABLE 9
Exemplary polynucleotide sequences encoding secretory signals

| | | SEQ ID |
|---|---|---|
| Signal | Sequence (Nucleotide) | NO: |
| HSV-1 gD | AUGGGGGGGGCUGCCGCCAGGUUGGGGGCCGUGAUUU | 121 |
| wild-type | UGUUUGUCGUCAUAGUGGGCCUCCAUGGGGUCCGCAG | |
| | CAAAUAU | |
| HSV-1 gD | AUGGGAGGAGCCGCCGCCAGACUGGGAGCCGUGAUCC | 122 |
| optimized | UGUUCGUGGUGAUCGUGGGACUGCAUGGAGUGAGAA | |
| nucleotide | GCAAGUAC | |
| sequence | | |
| Variation 3 | | |
| SARS-COV- | AUGUUUGUGUUUCUUGUGCUGCUGCCUCUUGUGUCUU | 123 |
| 2-S | CUCAGUGUGUGAAUUUGACA | |
| human Ig | AUGGAUUGGAUUUGGAGAAUCCUGUUCCUCGUGGGA | 124 |
| heavy chain | GCCGCUACAGGAGCCCACUCCCAGAUG | |
| secretory | | |
| signal (huSec) | | |
| human Ig | AUGGAGUUGGGACUGAGCUGGAUUUUCCUUUUGGCU | 125 |
| heavy chain | AUUUUAAAAGGUGUCCAGUGU | |
| secretory | | |
| signal | | |
| human Ig | AUGGAACUGGGGCUCCGCUGGGUUUUCCUUGUUGCUA | 126 |
| heavy chain | UUUUAGAAGGUGUCCAGUGU | |
| secretory | | |
| signal | | |
| human Ig | AUGAAACACCUGUGGUUCUUCCUCCUGCUGGUGGCAG | 127 |
| heavy chain | CUCCCAGAUGGGUCCUGUCC | |
| secretory | | |
| signal | | |
| human Ig | AUGGACUGGACCUGGAGGAUCCUCUUCUUGGUGGCAG | 128 |
| heavy chain | CAGCAACAGGUGCCCACUCG | |
| secretory | | |
| signal | | |
| human Ig | AUGGACUGGACCUGGAGGUUCCUCUUUGUGGUGGCAG | 129 |
| heavy chain | CAGCUACAGGUGUCCAGUCC | |
| secretory | | |
| signal | | |
| human Ig | AUGGAGUUUGGGCUGAGCUGGCUUUUUCUUGUGGCG | 130 |
| heavy chain | AUUCUAAAAGGUGUCCAGUGU | |
| secretory | | |
| signal | | |
| human Ig | AUGGAGUUUGGGCUGAGCUGGGUUUUCCUCGUUGCUC | 131 |
| heavy chain | UUUUUAGAGGUGUCCAGUGU | |
| secretory | | |
| signal | | |
| human Ig | AUGGACCUCCUGCACAAGAACAUGAAACACCUGUGGU | 132 |
| heavy chain | UCUUCCUCCUCCUGGUGGCAGCUCCCAGAUGGGUGCU | |
| secretory | GUCC | |
| signal | | |
| human Ig | AUGGACAUGAGGGUCCCUGCUCAGCUCCUGGGGCUCC | 133 |
| kappa chain | UGCUGCUCUGGCUCUCAGGUGCCAGAUGU | |
| secretory | | |
| signal | | |
| human Ig | AUGAAAUACCUAUUGCCUACGGCAGCCGCUGGAUUGU | 134 |
| kappa chain | UAUUACUCGCGGCCCAGCCGGCCAUGGCC | |
| secretory | | |
| signal | | |
| HSV-2 gI | AUGCCAGGACGGAGCCUUCAGGGCUUGGCCAUACUGG | 135 |
| Variation 2 | GGCUUUGGGUGUGUGCAACCGGGUUGGUAGUUCGA | |
| HSV-2 gI | AUGCCUGGCAGAUCUCUGCAAGGACUGGCCAUCCUCG | 136 |
| Variation 1 | GACUGUGGGUUUGCGCAACAGGCCUGGUUGUUAGA | |
| HSV-2 gI | AUGCCUGGAAGAUCUCUGCAGGGACUGGCAAUUCUGG | 137 |
| Variation 3 | GACUGUGGGUGUGUGCAACAGGACUGGUGGUGAGA | |
| HSV-1 gD | AUGGGAGGCGCAGCUGCCAGACUUGGUGCUGUGAUCC | 138 |
| | UGUUCGUGGUGAUUGUAGGGCUGCAU | |
| HSV-1 gD_ | AUGGGAGGCGCAGCUGCCAGACUUGGUGCUGUGAUCC | 139 |
| add | UGUUCGUGGUGAUUGUAGGGCUGCAUGGUGUCAGGG | |
| | GCAAGUAU | |
| HSV-2 gDwt | AUGGGCCGCCUGACCUCCGGCGUGGGCACCGCCGCCC | 140 |
| | UGCUGGUGGUGGCCGUGGGCCUGCGCGUGGUGUGCGC | |
| | C | |
| HSV-2 gD2 | AUGGGCAGACUGACAUCUGGCGUGGGAACAGCUGCUC | 141 |
| Variant 1 | UGCUGGUGGUUGCUGUGGGCCUGAGAGUCGUGUGUG | |
| | CC | |
| HSV-2 gD | AUGGGGAGACUCACAUCAGGCGUAGGAACCGCUGCCC | 142 |
| Variant 2 | UGUUGGUCGUGGCCGUUGGUCUGAGAGUUGUGUGUG | |
| | CC | |
| HSV-2 gD | AUGGGCAGACUGACCUCCGGCGUGGGCACCGCCGCCC | 143 |
| Variant 3 | UGCUGGUGGUGGCCGUGGGCCUGAGAGUGGUGUGCGC | |
| | C | |
| HSV-2 gD | AUGGGCCGCCUGACCUCCGGCGUGGGCACCGCCGCCC | 144 |
| KYA wt | UGCUGGUGGUGGCCGUGGGCCUGCGCGUGGUGUGCGC | |
| | CAAGUACGCC | |
| HSV-2 gD_ | AUGGGCAGACUGACAUCUGGCGUGGGAACAGCUGCUC | 145 |
| KYA | UGCUGGUGGUUGCUGUGGGCCUGAGAGUCGUGUGUG | |
| Variant 1 | CC | |
| HSV-2 gD_ | AUGGGGAGACUCACAUCAGGCGUAGGAACCGCUGCCC | 146 |
| KYA | UGUUGGUCGUGGCCGUUGGUCUGAGAGUUGUGUGUG | |
| Variant 2 | CCAAAUAUGCU | |
| HSV-2 | AUGGGCAGACUGACCUCCGGCGUGGGCACCGCCGCCC | 147 |
| gDKYA | UGCUGGUGGUGGCCGUGGGCCUGAGAGUGGUGUGCGC | |
| Variant 3 | C | |
| HSV-2 gD_ | AUGGGCAGACUGACAUCUGGCGUGGGAACAGCUGCUC | 148 |
| KYAL | UGCUGGUGGUUGCUGUGGGCCUGAGAGUCGUGUGUG | |
| Variant 1 | CCAAAUACGCCCUG | |
| HSV-2 gD_ | AUGGGGAGACUCACAUCAGGCGUAGGAACCGCUGCCC | 149 |
| KYAL | UGUUGGUCGUGGCCGUUGGUCUGAGAGUUGUGUGUG | |
| Variant 2 | CCAAAUAUGCUCUG | |
| HSV-2 gD_ | AUGGGCAGACUGACCUCCGGCGUGGGCACCGCCGCCC | 150 |
| KYAL | UGCUGGUGGUGGCCGUGGGCCUGAGAGUGGUGUGCGC | |
| Variant 3 | CAAAUACGCCCUG | |
| HSV-2 gC | AUGGCCUUGGGGAGAGUGGGCCUUGCAGUGGGUCUG | 151 |
| Variant 2 | UGGGGACUGCUCUGGGUUGGCGUAGUCGUCGUGCUGG | |
| | CUAACGCA | |
| IL2 | AUGCGCAUGCAGCUGCUGCUGCUGAUCGCCCUGUCCC | 152 |
| Wild type | UGGCCCUGGUGACCAACUCC | |
| IL2 | AUGAGAAUGCAGCUGCUGCUCCUGAUCGCCCUGUCUC | 153 |
| Variant 1 | UGGCCCUGGUCACCAAU | |
| IL2 | AUGCGCAUGCAACUGCUCCUGCUGAUUGCGUUGAGCC | 154 |
| Variant 2 | UUGCCCUGGUGACCAACAGC | |
| IL2 | AUGAGAAUGCAGCUGCUGCUGCUGAUCGCCCUGUCCC | 155 |
| Variant 3 | UGGCCCUGGUGACCAACUCC | |
| 12 Variant 1 | AUGCCUGGCAGAUCUCUGCAAGGACUGGCCAUCCUCG | 156 |
| | GACUGUGGGUUUGCGCAACAGGCCUG | |
| I2 | AUGCCAGGACGGAGCCUUCAGGGCUUGGCCAUACUGG | 157 |
| Variant 2 | GGCUUUGGGUGUGUGCAACCGGGUCU | |
| 12 | AUGCCUGGAAGAUCUCUGCAGGGACUGGCAAUUCUGG | 158 |
| Variant 3 | GACUGUGGGUGUGUGCAACAGGACUG | |
| HSV-2 gE | AUGGCACGGGGAGCCGGAUUGGUGUUCUUUGUGGGC | 159 |
| Variant 2 | GUGUGGGUGGUGAGCUGCUUGGCAGCCGCACCACGCA | |
| | CCUCU | |
| HSV-1 gD | AUGGGGGGGGCUGCCGCCAGGUUGGGGGCCGUGAUUU | 218 |
| (strain F) | UGUUUGUCGUCAUAGUCGGCCUCCAUGGGGUCCGCGG | |
| | C | |
| HSV-1 gD | AUGGGGCGUUUGACCUCCGGCGUCGGGACGGCGGCCC | 219 |
| (strain F) | UGCUAGUUGUCGCGGUGGGACUCCGCGUCGUCUGCGC | |
| Variant 1 | C | |

### C. Transmembrane Regions

In some embodiments, an HSV (e.g., HSV-1, HSV-2, or both) antigen construct as described herein includes a transmembrane region. In some embodiments, a transmembrane region is located at the N-terminus of a HSV (e.g., HSV-1, HSV-2, or both) construct. In some embodiments, a transmembrane region is located at the C-terminus of a HSV (e.g., HSV-1, HSV-2, or both) construct. In some embodiments, a transmembrane region is not located at the N-terminus or C-terminus of a HSV (e.g., HSV-1, HSV-2, or both) construct.

Transmembrane regions are known in the art, any of which can be utilized in a HSV (e.g., HSV-1, HSV-2, or both) construct described herein. In some embodiments, a transmembrane region comprises or is a transmembrane domain of Hemagglutinin (HA) of Influenza virus, Env of HIV-1, equine infectious anaemia virus (EIAV), murine leukaemia virus (MLV), mouse mammary tumor virus, G protein of vesicular stomatitis virus (VSV), Rabies virus, or a seven transmembrane domain receptor.

In some embodiments, a heterologous transmembrane region does not comprise a hemagglutin transmembrane region. In some embodiments, a heterologous transmembrane region comprises or consists of a non-human transmembrane region. In some embodiments, a heterologous transmembrane region comprises or consists of a viral transmembrane region. In some embodiments, a heterologous transmembrane region comprises or consists of an HSV transmembrane region, e.g., an HSV-1 or HSV-2 transmembrane region. In some embodiments, an HSV transmembrane region comprises or consists of an HSV gD transmembrane region, e.g., comprising or consisting of an amino acid sequence of

(SEQ ID NO: 160)
GLIAGAVGGSLLAALVICGIVYWMRRHTQKAPKRIRLPHIR.

In some embodiments, a heterologous transmembrane region comprises or consists of a human transmembrane region. In some embodiments, a human transmembrane region comprises or consists of a human decay accelerating factor glycosylphosphatidylinositol (hDAF-GPI) anchor region. In some embodiments, an hDAF-GPI anchor region comprises or consists of an amino acid sequence of

(SEQ ID NO: 161)
PNKGSGTTSGTTRLLSGHTCFTLTGLLGTLVTMGLLT.

In some embodiments, a utilized transmembrane region is a heterologous transmembrane region.

In some embodiments, a HSV (e.g., HSV-1, HSV-2, or both) construct described herein does not comprise a transmembrane region.

Exemplary transmembrane are provided in the following Table 9:

TABLE 10
Exemplary transmembrane regions

| SEQ | Trans- | |
|---|---|---|
| ID | membrane | |
| NO: | Region | Sequence (Amino Acid) |
| 160 | HSV-1 gD | GLIAGAVGGSLLAALVICGIVYWMRRHTQKAPKR |
| | | IRLPHIR |
| 161 | hDAF-GPI | PNKGSGTTSGTTRLLSGHTCFTLTGLLGTLVTMG |
| | anchor | LLT |
| | region | |

### D. Multimerization Regions

In some embodiments, an HSV (e.g., HSV-1, HSV-2, or both) construct as described herein includes one or more multimerization regions (e.g., a heterologous multimerization region).

In some embodiments, a heterologous multimerization region comprises a dimerization, trimerization or tetramerization region.

In some embodiments, a multimerization region is one described in WO2017/081082, which is incorporated herein by reference in its entirety (e.g., SEQ ID NOs: 1116-1167, or fragments or variants thereof). Exemplary trimerization and tetramerization regions include, but are not limited to, engineered leucine zippers, fibritin foldon domain from enterobacteria phage T4, GCN4pll, GCN4-pll, and p53.

In some embodiments, a provided HSV (e.g., HSV-1, HSV-2, or both) construct described herein is able to form a trimeric complex. For example, a provided HSV (e.g., HSV-1, HSV-2, or both) construct may comprise a multimerization region allowing formation of a multimeric complex, such as for example a trimeric complex of a HSV (e.g., HSV-1, HSV-2, or both) construct described herein. In some embodiments, a multimerization region allowing formation of a multimeric complex comprises a trimerization region, for example, a trimerization region described herein. In some embodiments, a HSV (e.g., HSV-1, HSV-2, or both) construct includes a T4-fibritin-derived “foldon” trimerization region, for example, to increase its immunogenicity. In some embodiments, a HSV (e.g., HSV-1, HSV-2, or both) construct includes a multimerization region comprising or consisting of the amino acid sequence

(SEQ ID NO: 162)
GYIPEAPRDGQAYVRKDGEWVLLSTFL.

### E. Linkers

In some embodiments, a HSV (e.g., HSV-1, HSV-2, or both) construct described herein includes one or more linkers. In some embodiments, a linker is or comprises 2, 3, 4, 5, 6, 7, 8, 9, 10 or more amino acids. In some embodiments, a linker is or comprises no more than about 30, 25, 20, 15, 10 or fewer amino acids. A linker can include any amino acid sequence and is not limited to any particular amino acids. In some embodiments, a linker comprises one or more glycine (G) amino acids. In some embodiments, a linker comprises one or more serine (S) amino acids. In some embodiments, a linker includes amino acids selected based on a cleavage predictor to generate highly-cleavable linkers.

In some embodiments, a linker is or comprises S-G₄-S-G₄-S(SEQ ID NO: 163). In some embodiments, a linker is or comprises GSPGSGSGS (SEQ ID NO: 164). In some embodiments, a linker is or comprises GGSGGGGSGG (SEQ ID NO: 165). In some embodiments, a linker is one presented in Table 10. In some embodiments, a linker is or comprises a sequence as set forth in WO2017/081082, which is incorporated herein by reference in its entirety (see SEQ ID NOs: 1509-1565, or a fragment or variant thereof).

In some embodiments, a HSV (e.g., HSV-1, HSV-2, or both) construct described herein comprises a linker between a C-terminal region or fragment thereof and a transmembrane region. In some embodiments, a HSV (e.g., HSV-1, HSV-2, or both) construct described herein comprises a linker after a minor repeat sequence.

Exemplary linkers are provided in the following Table 10:

TABLE 11
Exemplary linkers

| Linker | Sequence (Amino Acid) | SEQ ID NO |
|---|---|---|
| GSS | SGGGGSGGGGS | 163 |
| GSP | GSPGSGSGS | 164 |
| GS | GSGSGS | 217 |
| GGS | GGSGGGGSGG | 165 |
| From GGSL | GGSLGGGGSG | 166 |
| | GGGGSGGGGS | 167 |
| | GGS | 168 |
| | GGGS | 169 |
| | GGGGSGGGGSGGGGS | 170 |
| Furin | AGNRVRRSVG | 171 |
| SGG | SGG | 172 |

### F. MHC Class I Trafficking Signal (MITD)

In some embodiments, an HSV (e.g., HSV-1 and/or HSV-2) antigen for use in accordance with the present disclosure includes a trafficking signal. In some embodiments, a polyribonucleotide encoding one or more HSV (e.g., HSV-1 and/or HSV-2) antigens provided herein includes a trafficking signal. For example, in some embodiments, a trafficking signal is an MHC class I trafficking signal (MITD). In some embodiments, the MITID comprises or consists of an amino acid sequence of IVGIVAGLAVLAVVVIGAVVATVMCRRKSSGGKGGSYSQAASSDSAQGSDVSLTA (SEQ ID NO: 173). In some embodiments, the MITID comprises or consists of an amino acid sequence that is at least 80%, 85%, 90%, 91%, 92%, 93%, 94%, 95%, 96%, 97%, 98%, or 99% identical to

(SEQ ID NO: 173)
IVGIVAGLAVLAVVVIGAVVATVMCRRKSSGGKGGSYSQAASSDSAQGSD
VSLTA.

### G. Helper Antigens

In some embodiments, an HSV (e.g., HSV-1 and/or HSV-2) antigen for use in accordance with the present disclosure includes one or more helper antigens. In some embodiments, a polyribonucleotide encoding one or more HSV (e.g., HSV-1 and/or HSV-2) antigens provided herein includes one or more helper antigens. Those skilled in the art are aware that effective B cell responses often require assistance from helper T cells. As noted herein, it is proposed that an effective HSV (e.g., HSV-1 and/or HSV-2) vaccine, particularly if it targets HSV (e.g., HSV-1 and/or HSV-2) protein(s) (e.g., envelope protein(s), tegument protein(s), membrane protein(s), or combination thereof) expressed prior to cellular infection, may benefit from or require ability to induce particularly robust antibody response. In some embodiments, it may be desirable for a provided pharmaceutical composition (e.g., immunogenic composition, e.g., vaccine) to include or deliver one or more “helper antigens” (i.e., CD4 T cell antigens), for example in addition to one or more B-cell antigens and/or epitopes, and/or one or more T cell antigens and/or epitopes.

The present disclosure proposes that helper antigen(s) may be particularly useful when a pharmaceutical composition (e.g., immunogenic composition, e.g., vaccine) includes or delivers an antigen or an antigenic fragment thereof that includes repeated elements. In some embodiments, a provided pharmaceutical composition (e.g., immunogenic composition, e.g., vaccine) does not include or deliver an antigen that includes such repeated elements. Regardless, in some embodiments, a provided pharmaceutical composition (e.g., immunogenic composition, e.g., vaccine) does not include or deliver any helper antigen(s), or at least not helper antigen(s) (e.g., heterologous helper element(s)) specifically engineered into an antigen.

Where helper antigen(s) are desired, those skilled in the art are aware of a variety of potentially useful sequences, including those for example discussed in WO2020128031 which include, for instance, helper antigen(s) derived from P2 tetanus toxin, PADRE helper epitope, Hepatitis B surface antigen (HBsAg).

### III. Polyribonucleotides

In many embodiments, provided pharmaceutical compositions (e.g., immunogenic compositions, e.g., vaccines) deliver antigens as described herein by delivering a nucleic acid construct, e.g., in many embodiments, an RNA construct, that encodes one or more antigens as described herein and is expressed in the subject upon administration of the pharmaceutical composition (e.g., immunogenic composition, e.g., vaccine).

Among other things, the present disclosure encompasses the recognition that administration of nucleic acid, and particularly of RNA to achieve delivery (e.g., by expression) of encoded antigen can provide a variety of benefits relative to other strategies for immunizing against an HSV (e.g., HSV-1 and/or HSV-2) infection.

Among other things, the present disclosure provides an insight that RNA may be particularly useful and/or effective as an active agent in pharmaceutical compositions (e.g., immunogenic compositions, e.g., HSV (e.g., HSV-1 and/or HSV-2) vaccines) for a variety of reasons including specifically that RNA can have intrinsic adjuvanticity. As noted herein, ability to induce very high antibody titers to HSV (e.g., HSV-1 and/or HSV-2) proteins, e.g., particularly those expressed and/or targeted prior to cell invasion.

Still further, experience with SARS-CoV-2 vaccines has demonstrated that RNA actives can also elicit significant and diverse T cell responses which, particularly when combined with strong antibody response, represents a combination of immune characteristics thought to potentially maximize the probability of protection.

TABLE 12
Exemplary polyribonucleotide antigen sequences

| SEQ | | | |
|---|---|---|---|
| ID | | | |
| NO | Antigen | Optimized | Nucleotide Sequence |
| 75 | US7 | Opt1 | ggccccacagugucccugguguccgauucucuuguggaugcuggcgcugugggc |
| | [HSV-2 | | ccucagggauucguggaagaagaucugagagucuuuggcgagcugcacuucgug |
| | gI] | | ggagcccaggugccacacaccaacuacuacgacggcaucaucgagcuguuucacua |
| | No | | cccucugggcaaucacugccccagaguggugcacguggucacccuuacagccugu |
| | secretory | | ccuagaaggccugccguggccuucacacugugcagaucuacacaccacgcucacag |
| | signal | | ccccgccuauccuacacuggaacugggacuugccagacagccccugcugagaguc |
| | (antigenic | | agaaccgccaccagagauuacgccggccuguacguucugagagugugggucggaa |
| | region | | gcgccaccaacgccagcagauuugugcugggaguugcccugagcgccaacggcac |
| | only) | | cuuuguguacaacggcagegauuacggcagcugcgauccugcucagcugccuuuu |
| | | | agcgccccuagacugggcccuagcageguuuacacaccuggcgccucuagacccac |
| | | | accuccuagaacaacaaccccuccuagcagccccagagaucccacaccagcuccagg |
| | | | cgauacuggaacuccugcuccugccucuggegagaucgccccuccuaauucuaca |
| | | | agaagcgccagcgagagccggcacagaugauaa |
| 76 | US7 | Opt2 | ggccccacagugucccugguguccgauucucuuguggaugcuggcgcugugggc |
| | [HSV-2 | | ccucagggauucguggaagaagaucugagagucuuuggcgagcugcacuucgug |
| | gI] | | ggagcccaggugccacacaccaacuacuacgacggcaucaucgagcuguuucacua |
| | No | | cccucugggcaaucacugccccagaguggugcacguggucacccuuacagccugu |
| | secretory | | ccuagaaggccugccguggccuucacacugugcagaucuacacaccacgcucacag |
| | signal | | ccccgccuauccuacacuggaacugggacuugccagacagccccugcugagaguc |
| | (antigenic | | agaaccgccaccagagauuacgccggccuguacguucugagagugugggucggaa |
| | region | | gcgccaccaacgccagcagauuugugcugggaguugcccugagcgccaacggcac |
| | only) | | cuuuguguacaacggcagcgauuacggcagcugegauccugcucagcugccuuuu |
| | | | agcgccccuagacugggcccuagcageguuuacacaccuggcgccucuagacccac |
| | | | accuccuagaacaacaaccccuccuagcagccccagagaucccacaccagcuccagg |
| | | | cgauacuggaacuccugcuccugccucuggcgagaucgccccuccuaauucuaca |
| | | | agaagcgccagcgagagccggcacagaugauaa |
| 77 | US7 | Opt3 | gguccuaccgugucucucgugucugacagccugguagacgccggugcugugggc |
| | [HSV-2 | | ccucagggcuucguugaggaggaucugcggguguuuggggaauugcacuuugu |
| | gI] | | gggcgcucagguuccgcacaccaacuacuacgaugggaucaucgagcuguuccac |
| | No | | uauccccuugggaaccauugcccgagagucgugcauguggucacccugaccgcuu |
| | secretory | | gcccucgccgaccagcagucgccuuuacccugugucgcagcacucaccaugcccau |
| | signal | | aguccagcauaucccacucuggaacugggacuggccaggcaaccacugcugagag |
| | (antigenic | | ugcgcacagccacacgggacuaugccggacuguaugugcucagagucuggguug |
| | region | | ggagcgcaaccaaugcgucacguuuugugcucggaguagcccucuccgccaaugg |
| | only) | | uacguucgucuacaacggcuccgauuacggcagcugcgauccagcgcagcugcca |
| | | | uucucagcacccaggcucggucccucaagcgucuacacaccaggcgcuucuagacc |
| | | | cacuccacccaggacuacgacaccucccuccaguccucgugaccccacaceggcuc |
| | | | cuggagacaccggaacucccgcuccugcgucuggegagauugcaccucccaauuc |
| | | | cacacggagugccagcgaauccaggcaccgcugauaa |
| 78 | US7 | Opt4 | ggaccaacagugucucuggugucugauucucugguggacgcuggagcuguggga |
| | [HSV-2 | | ccucagggauuuguggaagaagaucugagaguguuuggagaacugcacuuugug |
| | gI] | | ggagcucaggugccucacacaaauuauuaugacggaaucaucgaacuguuucacu |
| | No | | acccucugggaaaucacugcccaagaguggugcauguggugacacugacagcuug |
| | secretory | | cccaagaagaccugcuguggcuuuuacacugugcagaucaacacaccacgcucacu |
| | signal | | cuccagcuuacccaacacuggaacugggucuggcaagacagccucugcugagagu |
| | (antigenic | | gagaacagcaacaagagauuaugcuggacuguaugugcugagaguguggguggg |
| | region | | aucugcaacaaaugcuucaagauuugugcugggaguggcucugucugcaaaugg |
| | only) | | aacauuuguguacaauggaucugauuauggaucuugugauccugcucagcugcc |
| | | | uuuuucugcuccaagacugggaccuucuucuguguacacaccuggagcuucaaga |
| | | | ccaacaccaccaagaacaacaacaccuccuucuucuccaagagauccaacaccugcu |
| | | | ccuggagacacaggaacaccugcuccugcuucuggagaaauugcuccuccaaauu |
| | | | caacgagaucugcuucugaaucaagacacagaugauaa |
| 79 | US7 | Opt10 | ggaccaacagugucucuggugucugauucucugguggacgcuggagcuguggga |
| | [HSV-2 | | ccucagggauuuguggaagaagaucugagaguguuuggagaacugcacuuugug |
| | gI] | | ggagcucaggugccucacacaaauuauuaugacggaaucaucgaacuguuucacu |
| | No | | acccucugggaaaucacugcccaagaguggugcauguggugacacugacagcuug |
| | secretory | | cccaagaagaccugcuguggcuuuuacacugugcagaucaacacaccacgcucacu |
| | signal | | cuccagcuuacccaacacuggaacugggucuggcaagacagccucugcugagagu |
| | (antigenic | | gagaacagcaacaagagauuaugcuggacuguaugugcugagaguguggguggg |
| | region | | aucugcaacaaaugcuucaagauuugugcugggaguggcucugucugcaaaugga |
| | only) | | acauuuguguacaauggaucugauuauggaucuugugauccugcucagcugccu |
| | | | uuuucugcuccaagacugggaccuucuucuguguacacaccuggagcuucaagac |
| | | | caacaccaccaagaacaacaacaccuccuucuucuccaagagauccaacaccugcuc |
| | | | cuggagacacaggaacaccugcuccugcuucuggagaaauugcuccuccaaauuc |
| | | | aacgagaucugcuucugaaucaagacacagaugauaa |
| 80 | US8 | wt | cgcaccuccuggaagcgcgugaccuccggcgaggacguggugcugcugcccgccc |
| | [HSV-2 | | ccgccggccccgaggagcgcacccgcgcccacaagcugcugugggccgccgagccc |
| | gE] | | cuggacgccugcggcccccugcgccccuccuggguggcccuguggcccccccgcc |
| | No | | gcgugcuggagaccgugguggacgccgccugcaugcgcgcccccgagccccuggc |
| | secretory | | caucgccuacuccccccccuuccccgccggcgacgagggccuguacuccgagcugg |
| | signal | | ccuggcgcgaccgcguggccguggugaacgagucccuggugaucuacggcgcccu |
| | (antigenic | | ggagaccgacuccggccuguacacccuguccguggugggccuguccgacgaggcc |
| | region | | cgccagguggccuccguggugcuggugguggagcccgcccccgugcccaccccca |
| | only) | | cccccgacgacuacgacgaggaggacgacgccggcguguccgagcgcacccccgug |
| | | | uccgugcccccccccacccccccccgccgcccccccguggccccccccacccacccc |
| | | | cgcgugauccccgaggugucccacgugcgcggcgugaccgugcacauggagaccc |
| | | | ccgaggccauccuguucgcccccggcgagaccuucggcaccaacguguccauccac |
| | | | gccaucgcccacgacgacggccccuacgccauggacgugguguggaugcgcuucg |
| | | | acgugcccuccuccugegccgagaugegcaucuacgaggccugccuguaccacccc |
| | | | cagcugcccgagugccugucccccgccgacgcccccugcgccguguccuccuggg |
| | | | ccuaccgccuggccgugcgcuccuacgccggcugcuccegcaccaccccccccccc |
| | | | cgcugcuucgccgaggcccgcauggagcccgugcccggccuggccuggcuggccu |
| | | | ccaccgugaaccuggaguuccagcacgccuccccccagcacgccggccuguaccug |
| | | | ugcgugguguacguggacgaccacauccacgccuggggccacaugaccaucucca |
| | | | ccgccgcccaguaccgcaacgccgugguggagcagcaccugccccagegccagccc |
| | | | gagcccguggagcccacccgcccccacgugcgcgccuaa |
| 81 | US8 | Opt1 | agcagaaccagcuggaaaagagugaccagcggcgaggauguggugcugcuuccug |
| | [HSV-2 | | cuccugcuggccccgaggaaagaacaagagcccacaaacugcugugggccgcuga |
| | gE] | | gccucuugaugccuguggaccucucagaccuagcuggguugcacuguggccaccu |
| | No | | cggagagugcuggaaacagugguggaugccgccugcaugagagccccugaaccuc |
| | secretory | | uggccauugccuacucuccaccauuuccagccggcgacgagggccuguauucuga |
| | signal | | gcuugcuuggagagacagaguggccguggucaacgagagccugguuaucuaugg |
| | (antigenic | | cgcccuggaaaccgacagcggccuguacacacugucugucgugggccugucugac |
| | region | | gaggcuagacagguggcaucugugguccuggugguggaaccugcuccagugccu |
| | only) | | acaccuacaccugacgacuacgacgaggaagaugacgcuggcgucagcgagagaac |
| | | | cccuguuucugugccuccuccuacgccuccucguagaccuccuguugcuccucca |
| | | | acacaccccagagugaucccugaagugucucacgugcggggcgugaccgugcaca |
| | | | uggaaacaccugaggccauccuguucgccccuggcgagacauuuggcaccaacgu |
| | | | guccauccacgcuaucgcccacgacgauggcccuuacgccauggaugucgugugg |
| | | | augagauucgacgugcccagcagcugugccgagaugagaaucuaugaggccugcc |
| | | | uguaucacccucagcugcccgaaugucugagcccugcugaugccccuugugccgu |
| | | | uagcagcugggccuauagacuggccgugcggucuuaugccggcugcucuagaaca |
| | | | accccuccuccucgguguuucgccgaggccagaauggaaccuguuccuggacugg |
| | | | ccuggcuggccuccacagugaaccuggaauuucagcacgccucuccacagcacgcc |
| | | | ggccuguaucugugugugguguacguggacgaucacauccacgccuggggccaca |
| | | | ugaccaucucuacagccgcucaguaccggaacgccgugguugaacagcaucugcc |
| | | | ucagagacagcccgagccuguggaaccuacaagaccucauguucgggccuga |
| 82 | US8 | Opt2 | cgcaccucuuggaaacgcguuacuuccggggaggacguuguccuccuuccagcac |
| | [HSV-2 | | ccgcaggaccugaagagaggacuagggcccacaagcugcugugggccgcugaacc |
| | gE] | | ucuggaugccugugguccucugagaccuagcugggucgcccuuuggccaccuaga |
| | No | | cgcguucuggagacggucguggaugccgcgugcaugcgugcacccgaaccucugg |
| | secretory | | ccaucgccuauagucccccuuuucccgcuggcgacgaggggcuuuacuccgaacu |
| | signal | | ggccuggcgggauaggguggcgguggugaacgagagccucgucaucuacggugc |
| | (antigenic | | ucuggaaaccgacucaggacuguauacgcucagcguuguuggccucuccgaugag |
| | region | | gcucgacagguugccuccguagugcuggucguagaaccagcccccguaccaacac |
| | only) | | ccacacccgacgacuacgacgaagaggacgacgcuggaguuagcgaaagaacaccg |
| | | | gugagugugccaccucccacacegccaaggagacccccaguagcaccuccaaccca |
| | | | uccgagagugauucccgaggucagccaugugcgcggcguaacugugcacauggag |
| | | | acgcccgaagcgauacuguuugccccuggagagacauucggcaccaaugugucca |
| | | | uacacgcaauugcgcacgaugauggcccauacgcuauggacgucgucuggaugag |
| | | | guucgaugugccuucuucuugcgccgagaugaggaucuacgaggcaugccugua |
| | | | ucacccccaauugccggagugucugucucccgcagaugcaccgugugcagugagu |
| | | | agcugggcuuaucgguuggcuguccggaguuaugcuggguguucacggaccacc |
| | | | ccaccuccacguugcuuugcugaagccagaauggaacccgugccuggucuggcuu |
| | | | ggcuggcaucaacugucaaccuggaguuccagcaugccucuccacagcacgcagg |
| | | | ccuguaucucugcgugguguacguugacgaucacauccaugcgugggggcauau |
| | | | gaccaucagcacagcugcccaguaccgcaaugccgucguggagcagcaccuccccc |
| | | | aacggcagccagaaccaguggagcccacucggccucaugugcgagccuga |
| 83 | US8 | Opt3 | aaauaugcucgcaccucuuggaaacgcguuacuuccggggaggacguuguccucc |
| | [HSV-2 | | uuccagcacccgcaggaccugaagagaggacuagggcccacaagcugcugugggc |
| | gE] | | cgcugaaccucuggaugccugugguccucugagaccuagcugggucgcccuuug |
| | No | | gccaccuagacgcguucuggagacggucguggaugccgcgugcaugcgugcaccc |
| | secretory | | gaaccucuggccaucgccuauagucccccuuuucccgcuggcgacgaggggcuuu |
| | signal | | acuccgaacuggccuggcgggauaggguggcgguggugaacgagagccucguca |
| | (antigenic | | ucuacggugcucuggaaaccgacucaggacuguauacgcucagcguuguuggccu |
| | region | | cuccgaugaggcucgacagguugccuccguagugcuggucguagaaccagccccc |
| | only) | | guaccaacacccacacccgacgacuacgacgaagaggacgacgcuggaguuagcga |
| | | | aagaacaccggugagugugccaccucccacacegccaaggagacccccaguagcac |
| | | | cuccaacccauccgagagugauucccgaggucagccaugugcgcggcguaacugu |
| | | | gcacauggagacgcccgaagcgauacuguuugccccuggagagacauucggcacc |
| | | | aauguguccauacacgcaauugcgcacgaugauggcccauacgcuauggacgucg |
| | | | ucuggaugagguucgaugugccuucuucuugcgccgagaugaggaucuacgagg |
| | | | caugccuguaucacccccaauugccggagugucugucucccgcagaugcaccgug |
| | | | ugcagugaguagcugggcuuaucgguuggcuguccggaguuaugcuggguguuc |
| | | | acggaccaccccaccuccacguugcuuugcugaagccagaauggaacccgugccug |
| | | | gucuggcuuggcuggcaucaacugucaaccuggaguuccagcaugccucuccaca |
| | | | gcacgcaggccuguaucucugcgugguguacguugacgaucacauccaugcgugg |
| | | | gggcauaugaccaucagcacagcugcccaguaccgcaaugccgucguggagcagc |
| | | | accucccccaacggcagccagaaccaguggagcccacucggccucaugugcgagcc |
| | | | uga |
| 84 | US8 | Opt4 | agaaccuccuggaaaagagugaccuccggcgaagauguggugcugcugcccgccc |
| | [HSV-2 | | ccgccggccccgaagaaagaaccagagcccacaaacugcugugggccgccgaaccc |
| | gE] | | cuggaugccugeggcccccucagacccuccuggguggcccuguggcccccuagaa |
| | No | | gggugcuggaaaccgugguggaugccgccugcaugagagcccccgaaccccuggc |
| | secretory | | caucgccuacuccccucccuuccccgccggegaugaaggccuguacuccgaacugg |
| | signal | | ccuggagagauagaguggccguggugaacgaaucccuggugaucuacggcgcccu |
| | (antigenic | | ggaaaccgauuccggccuguacacccuguccguggugggccuguccgaugaagcc |
| | region | | agacagguggccuccguggugcuggugguggaaccegcccccgugcccaccccca |
| | only) | | cccccgaugauuacgaugaagaagaugaugccggcguguccgaaagaacccccgu |
| | | | guccgugcccccucccaccccuccccgcagacccccuguggccccucccacccaccc |
| | | | cagagugauccccgaagugucccacgugagaggcgugaccgugcacauggaaacc |
| | | | cccgaagccauccuguucgcccccggcgaaaccuucggcaccaacguguccaucca |
| | | | cgccaucgcccacgaugauggccccuacgccauggaugugguguggaugagauuc |
| | | | gaugugcccuccuccugegccgaaaugagaaucuacgaagccugccuguaccaccc |
| | | | ccagcugcccgaaugccugucccccgccgaugcccccugcgccguguccuccugg |
| | | | gccuacagacuggccgugagauccuacgccggcugcuccagaaccaccccuccccc |
| | | | uagaugcuucgccgaagccagaauggaacccgugcccggccuggccuggcuggcc |
| | | | uccaccgugaaccuggaauuccagcacgccagcccccagcacgccggccuguaccu |
| | | | gugcgugguguacguggaugaucacauccacgccuggggccacaugaccaucucc |
| | | | accgccgcccaguacagaaacgccgugguggaacagcaccugccccagagacagcc |
| | | | cgaacccguggaacccaccagaccccacgugagagccuga |

### A. Exemplary Polyribonucleotides Features

Polyribonucleotides described herein encode one or more HSV (e.g., HSV-1, HSV-2, or both) constructs described herein. In some embodiments, polyribonucleotides described herein can comprise a nucleotide sequence that encodes a 5′UTR of interest and/or a 3′ UTR of interest. In some embodiments, polynucleotides described herein can comprise a nucleotide sequence that encodes a polyA tail. In some embodiments, polyribonucleotides described herein may comprise a 5′ cap, which may be incorporated during transcription, or joined to a polyribonucleotide post-transcription.

### 1. 5′ Cap

A structural feature of mRNAs is cap structure at five-prime end (5′). Natural eukaryotic mRNA comprises a 7-methylguanosine cap linked to the mRNA via a 5′ to 5′-triphosphate bridge resulting in cap0 structure (m7GpppN). In most eukaryotic mRNA and some viral mRNA, further modifications can occur at the 2′-hydroxy-group (2′-OH) (e.g., the 2′-hydroxyl group may be methylated to form 2′-O-Me) of the first and subsequent nucleotides producing “cap1” and “cap2” five-prime ends, respectively). Diamond, et al., (2014) Cytokine & growth Factor Reviews, 25:543-550, which is incorporated herein by reference in its entirety, reported that cap0-mRNA cannot be translated as efficiently as cap1-mRNA in which the role of 2′-O-Me in the penultimate position at the mRNA 5′ end is determinant. Lack of the 2′-O-met has been shown to trigger innate immunity and activate IFN response. Daffis, et al. (2010) Nature, 468:452-456; and Zust et al. (2011) Nature Immunology, 12:137-143, each of which is incorporated herein by reference in its entirety.

RNA capping is well researched and is described, e.g., in Decroly E et al. (2012) Nature Reviews 10: 51-65; and in Ramanathan A. et al., (2016) Nucleic Acids Res; 44(16): 7511-7526, the entire contents of each of which is hereby incorporated by reference. For example, in some embodiments, a 5′-cap structure which may be suitable in the context of the present invention is a cap0 (methylation of the first nucleobase, e.g. m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (“anti-reverse cap analogue”), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

The term “5′-cap” as used herein refers to a structure found on the 5′-end of an RNA, e.g., mRNA, and generally includes a guanosine nucleotide connected to an RNA, e.g., mRNA, via a 5′- to 5′-triphosphate linkage (also referred to as Gppp or G(5′)ppp(5′)). In some embodiments, a guanosine nucleoside included in a 5′ cap may be modified, for example, by methylation at one or more positions (e.g., at the 7-position) on a base (guanine), and/or by methylation at one or more positions of a ribose. In some embodiments, a guanosine nucleoside included in a 5′ cap comprises a 3′O methylation at a ribose (3′OMeG). In some embodiments, a guanosine nucleoside included in a 5′ cap comprises methylation at the 7-position of guanine (m7G). In some embodiments, a guanosine nucleoside included in a 5′ cap comprises methylation at the 7-position of guanine and a 3′ O methylation at a ribose (m7(3′OMeG)). It will be understood that the notation used in the above paragraph, e.g., “(m₂^{7,3′-O})G” or “m7(3′OMeG)”, applies to other structures described herein.

In some embodiments, providing an RNA with a 5′-cap disclosed herein may be achieved by in vitro transcription, in which a 5′-cap is co-transcriptionally expressed into an RNA strand, or may be attached to an RNA post-transcriptionally using capping enzymes. In some embodiments, co-transcriptional capping with a cap disclosed improves the capping efficiency of an RNA compared to co-transcriptional capping with an appropriate reference comparator. In some embodiments, improving capping efficiency can increase a translation efficiency and/or translation rate of an RNA, and/or increase expression of an encoded polypeptide. In some embodiments, alterations to polynucleotides generates a non-hydrolyzable cap structure which can, for example, prevent decapping and increase RNA half-life.

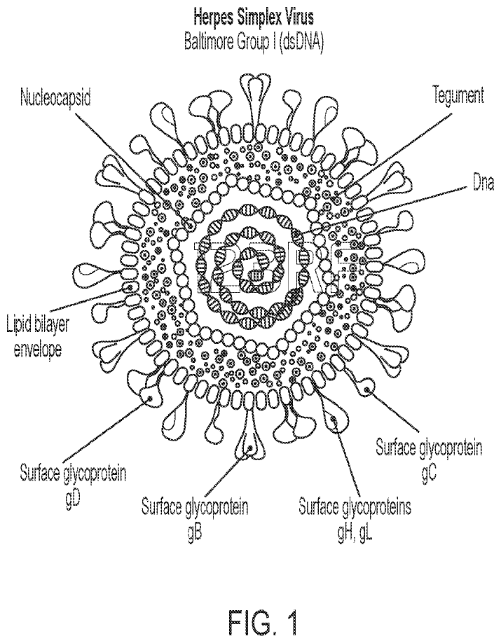

In some embodiments, a utilized 5′ caps is a cap0, a cap1, or cap2 structure. See, e.g., FIG. 1 of Ramanathan A et al., and FIG. 1 of Decroly E et al., each of which is incorporated herein by reference in its entirety. See, e.g., FIG. 1 of Ramanathan A et al., and FIG. 1 of Decroly E et al., each of which is incorporated herein by reference in its entirety. In some embodiments, an RNA described herein comprises a cap1 structure. In some embodiments, an RNA described herein comprises a cap2.

In some embodiments, an RNA described herein comprises a cap0 structure. In some embodiments, a cap0 structure comprises a guanosine nucleoside methylated at the 7-position of guanine ((m⁷)G). In some embodiments, such a cap0 structure is connected to an RNA via a 5′- to 5′-triphosphate linkage and is also referred to herein as (m⁷)Gppp. In some embodiments, a cap0 structure comprises a guanosine nucleoside methylated at the 2′-position of the ribose of guanosine. In some embodiments, a cap0 structure comprises a guanosine nucleoside methylated at the 3′-position of the ribose of guanosine. In some embodiments, a guanosine nucleoside included in a 5′ cap comprises methylation at the 7-position of guanine and at the 2′-position of the ribose ((m₂^{7,2′-O})G). In some embodiments, a guanosine nucleoside included in a 5′ cap comprises methylation at the 7-position of guanine and at the 2′-position of the ribose ((m₂^{7,3′-O})G).

In some embodiments, a cap1 structure comprises a guanosine nucleoside methylated at the 7-position of guanine ((m⁷)G) and optionally methylated at the 2′ or 3′ position pf the ribose, and a 2′O methylated first nucleotide in an RNA ((m^{2′-O})N₁). In some embodiments, a cap1 structure comprises a guanosine nucleoside methylated at the 7-position of guanine ((m⁷)G) and the 3′ position of the ribose, and a 2′O methylated first nucleotide in an RNA ((m^{2′-O})N₁). In some embodiments, a cap1 structure is connected to an RNA via a 5′- to 5′-triphosphate linkage and is also referred to herein as, e.g., ((m⁷)Gppp(^{2′-O})N₁) or (m₂^{7,3′-O})Gppp(^{2′-O})N₁), wherein N₁ is as defined and described herein. In some embodiments, a cap1 structure comprises a second nucleotide, N₂, which is at position 2 and is chosen from A, G, C, or U, e.g., (m⁷)Gppp(^{2′-O})N₁pN₂ or (m₂^{7,3′-O})Gppp(^{2′-O})N₁pN₂, wherein each of N₁ and N₂ is as defined and described herein.

In some embodiments, a cap2 structure comprises a guanosine nucleoside methylated at the 7-position of guanine ((m⁷)G) and optionally methylated at the 2′ or 3′ position pf the ribose, and a 2′O methylated first and second nucleotides in an RNA ((m^{2′-O})N₁p(m^{2′-O})N₂). In some embodiments, a cap2 structure comprises a guanosine nucleoside methylated at the 7-position of guanine ((m⁷)G) and the 3′ position of the ribose, and a 2′O methylated first and second nucleotide in an RNA. In some embodiments, a cap2 structure is connected to an RNA via a 5′- to 5′-triphosphate linkage and is also referred to herein as, e.g., ((m⁷)Gppp(^{2′-O})N₁p(^{2′-O})N₂) or (m₂^{7,3′-O})Gppp(^{2′-O})N₁p(^{2′-O})N₂), wherein each of N₁ and N₂ is as defined and described herein.

In some embodiments, the 5′ cap is a dinucleotide cap structure. In some embodiments, the 5′ cap is a dinucleotide cap structure comprising N₁, wherein N₁ is as defined and described herein. In some embodiments, the 5′ cap is a dinucleotide cap G*N₁, wherein N₁ is as defined above and herein, and:
G* comprises a structure of formula (I):

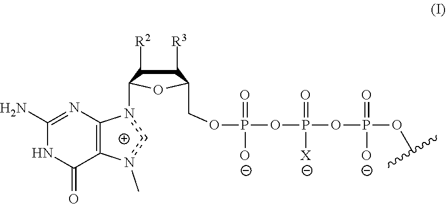

or a salt thereof,
wherein
each R² and R³ is —OH or —OCH₃; and
X is O or S.

In some embodiments, R² is —OH. In some embodiments, R² is —OCH₃. In some embodiments, R³ is —OH. In some embodiments, R³ is —OCH₃. In some embodiments, R² is —OH and R³ is —OH. In some embodiments, R² is —OH and R³ is —CH₃. In some embodiments, R² is —CH₃ and R³ is —OH. In some embodiments, R² is —CH₃ and R³ is —CH₃.

In some embodiments, X is O. In some embodiments, X is S.

In some embodiments, the 5′ cap is a dinucleotide cap0 structure (e.g., (m⁷)GpppN₁, (m₂^{7,2′-O})GpppN₁, (m₂^{7,3′-O})GpppN₁, (m⁷)GppSpN₁, (m₂^{7,2′-O})GppSpN₁, or (m₂^{7,3-O})GppSpN₁), wherein N₁ is as defined and described herein. In some embodiments, the 5′ cap is a dinucleotide cap0 structure (e.g., (m⁷)GpppN₁, (m₂^{7,2-O})GpppN₁, (m₂^{7,3′-O})GpppN₁, (m⁷)GppSpN₁, (m₂^{7,2′-O})GppSpN₁, or (m₂^{7,3′-O})GppSpN₁), wherein N₁ is G. In some embodiments, the 5′ cap is a dinucleotide cap0 structure (e.g., (m⁷)GpppN₁, (m₂^{7,2′-O})GpppN₁, (m₂^{7,3-O})GpppN₁, (m⁷)GppSpN₁, (m₂^{7,2′-O})GppSpN₁, or (m₂^{7,3′-O})GppSpN₁), wherein N₁ is A, U, or C. In some embodiments, the 5′ cap is a dinucleotide cap1 structure (e.g., (m⁷)Gppp(m^{2′-O})N₁, (m₂^{7,2′-O})Gppp(m^{2′-O})N₁, (m₂^{7,3-O})Gppp(m^{2′-O})N₁, (m⁷)GppSp(m^{2′-O})N₁, (m₂^{7,2′-O})GppSp(m^{2′-O})N₁, or (m₂^{7,3′-O})GppSp(m^{2′-O})N₁), wherein N₁ is as defined and described herein. In some embodiments, the 5′ cap is selected from the group consisting of (m⁷)GpppG (“Ecap0”), (m⁷)Gppp(m^{2′-O})G (“Ecap1”), (m₂^{7,3′-O})GpppG (“ARCA” or “D1”), and (m₂^{7,2′-O})GppSpG (“beta-S-ARCA”). In some embodiments, the 5′ cap is (m⁷)GpppG (“Ecap0”), having a structure:

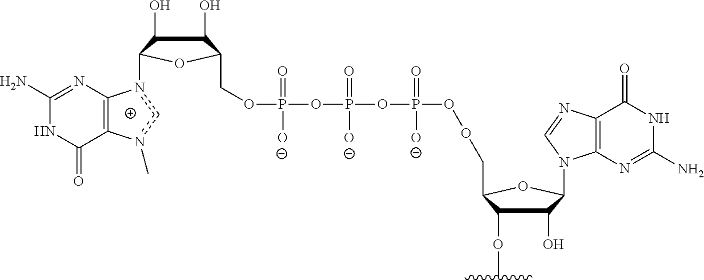

or a salt thereof.

In some embodiments, the 5′ cap is (m⁷)Gppp(m^{2′-O})G (“Ecap1”), having a structure:

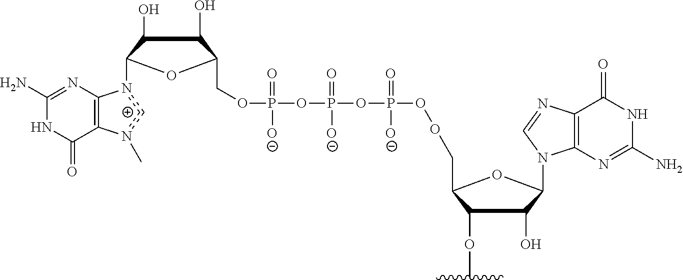

or a salt thereof.

In some embodiments, the 5′ cap is (m₂^{7,3′-O})GpppG (“ARCA” or “D1”), having a structure:

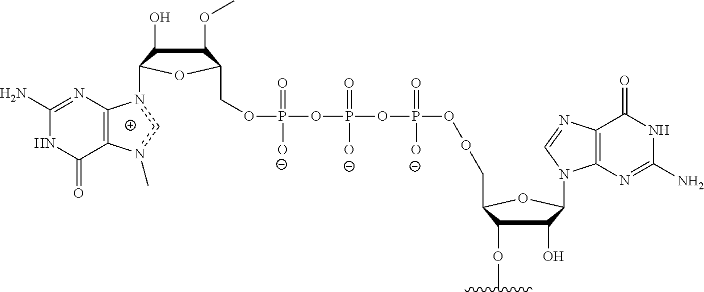

or a salt thereof.

In some embodiments, the 5′ cap is (m₂^{7,2′-O})GppSpG (“beta-S-ARCA”), having a structure:

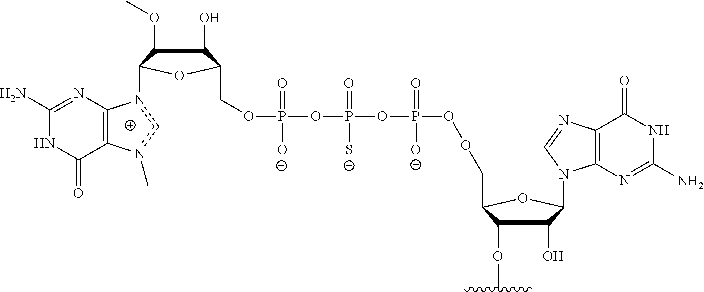

or a salt thereof.

In some embodiments, the 5′ cap is a trinucleotide cap structure. In some embodiments, the 5′ cap is a trinucleotide cap structure comprising N₁pN₂, wherein N₁ and N₂ are as defined and described herein. In some embodiments, the 5′ cap is a dinucleotide cap G*N₁pN₂, wherein N₁ and N₂ are as defined above and herein, and: G* comprises a structure of formula (I):

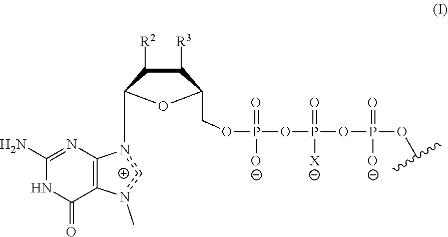

or a salt thereof, wherein R², R³, and X are as defined and described herein.

In some embodiments, the 5′ cap is a trinucleotide cap0 structure (e.g. (m⁷)GpppN₁pN₂, (m₂^{7,2′-O})GpppN₁pN₂, or (m₂^{7,3′-O})GpppN₁pN₂), wherein N₁ and N₂ are as defined and described herein). In some embodiments, the 5′ cap is a trinucleotide cap1 structure (e.g., (m⁷)Gppp(m^{2′-O})N₁pN₂, (m₂^{7,2′-O})Gppp(m^{2′-O})N₁pN₂, (m₂^{7,3′-O})Gppp(m^{2′-O})N₁pN₂), wherein N₁ and N₂ are as defined and described herein. In some embodiments, the 5′ cap is a trinucleotide cap2 structure (e.g., (m⁷)Gppp(m^{2′-O})N₁p(m^{2′-O})N₂, (m₂^{7,2′-O})Gppp(m^{2′-O})N₁p(m^{2′-O})N₂, (m₂^{7,3′-O})Gppp(m^{2′-O})N₁p(m^{2′-O})N₂), wherein N₁ and N₂ are as defined and described herein. In some embodiments, the 5′ cap is selected from the group consisting of (m₂^{7,3′-O})Gppp(m^{2′-O})ApG (“CleanCap AG”, “CC413”), (m₂^{7,3′-O})Gppp(m^{2′-O})GpG (“CleanCap GG”), (m⁷)Gppp(m^{2′-O})ApG, (m⁷)Gppp(m^{2′-O})GpG, (m₂^{7,3′-O})Gppp(m₂^{6,2′-O})ApG, and (m⁷)Gppp(m^{2′-O})ApU.

In some embodiments, the 5′ cap is (m₂^{7,3-O})Gppp(m^{2′-O})ApG (“CleanCap AG”, “CC413”), having a structure:

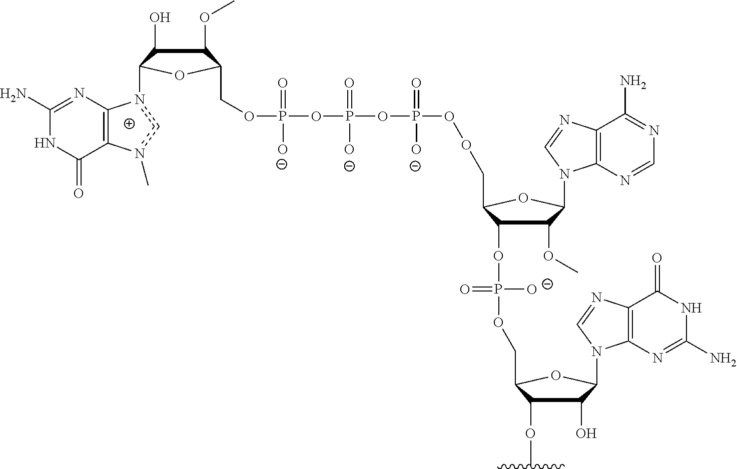

or a salt thereof.

In some embodiments, the 5′ cap is (m₂^{7,3′-O})Gppp(m^{2′-O})GpG (“CleanCap GG”), having a structure:

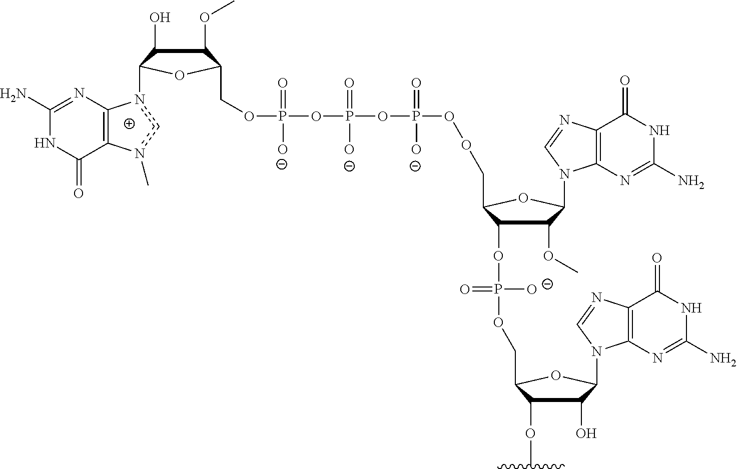

or a salt thereof.

In some embodiments, the 5′ cap is (m⁷)Gppp(m^{2′-O})ApG, having a structure:

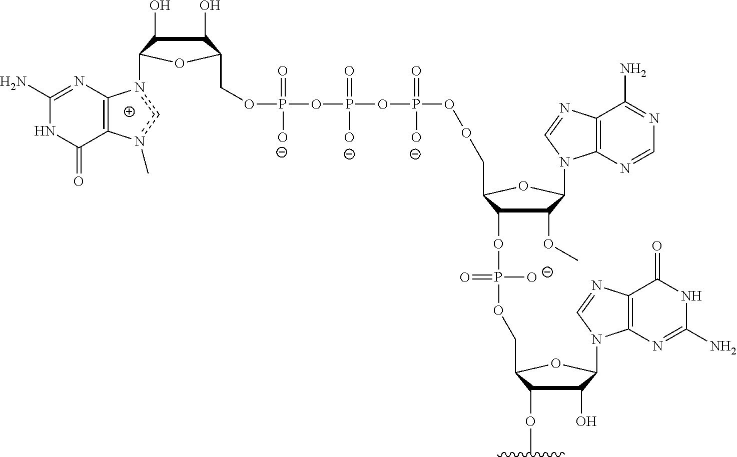

or a salt thereof.

In some embodiments, the 5′ cap is (m⁷)Gppp(m^{2′-O})GpG, having a structure:

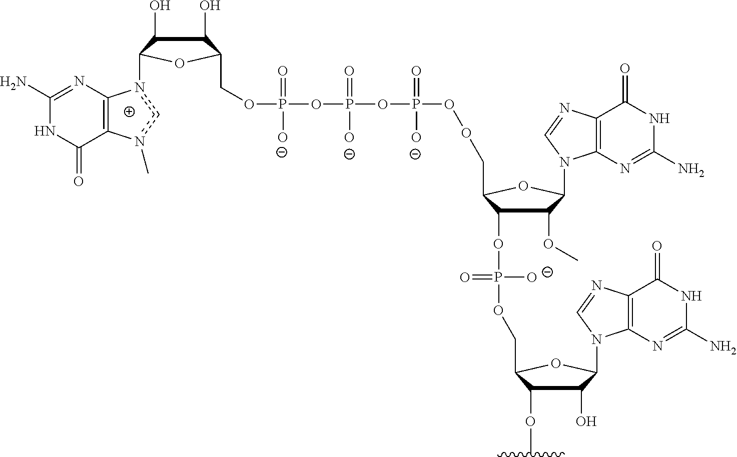

or a salt thereof.

In some embodiments, the 5′ cap is (m₂^{7,3′-O})Gppp(m₂^{6,2′-O})ApG, having a structure:

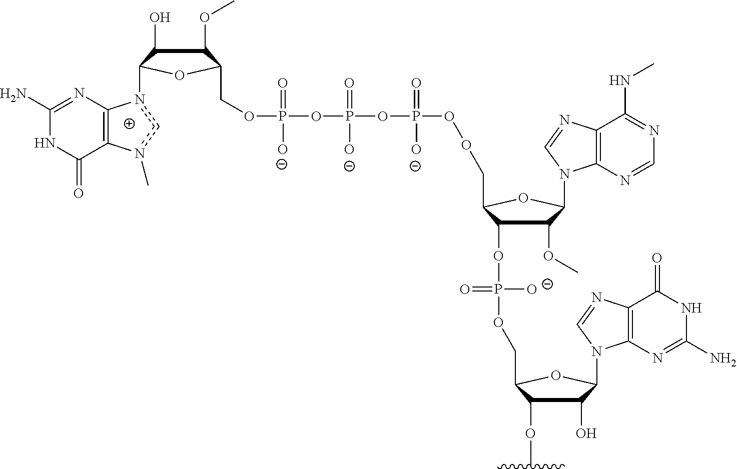

or a salt thereof.

In some embodiments, the 5′ cap is (m⁷)Gppp(m^{2′-O})ApU, having a structure:

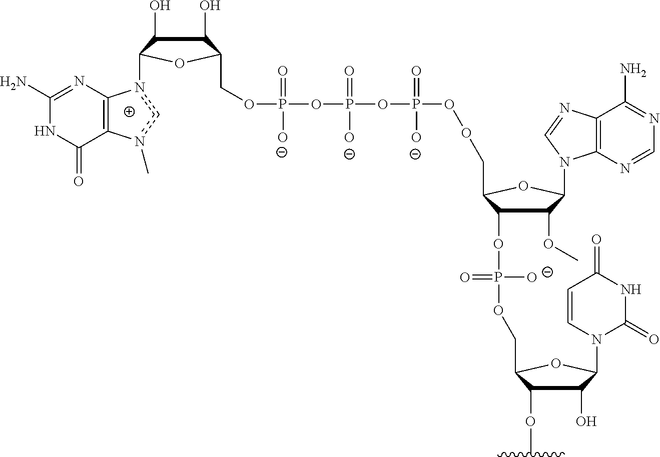

or a salt thereof.

In some embodiments, the 5′ cap is a tetranucleotide cap structure. In some embodiments, the 5′ cap is a tetranucleotide cap structure comprising N₁pN₂pN₃, wherein N₁, N₂, and N₃ are as defined and described herein. In some embodiments, the 5′ cap is a tetranucleotide cap G*N₁pN₂pN₃, wherein N₁, N₂, and N₃ are as defined above and herein, and:
G* comprises a structure of formula (I):

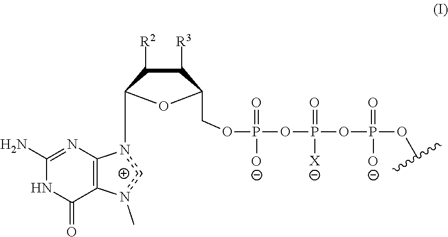

or a salt thereof, wherein R², R³, and X are as defined and described herein.

In some embodiments, the 5′ cap is a tetranucleotide cap0 structure (e.g. (m⁷)GpppN₁pN₂pN₃, (m₂^{7,2′-O})GpppN₁pN₂pN₃, or (m₂^{7,3′-O})GpppN₁N₂pN₃), wherein N₁, N₂, and N₃ are as defined and described herein). In some embodiments, the 5′ cap is a tetranucleotide Cap1 structure (e.g., (m⁷)Gppp(m^{2′-O})N₁pN₂pN₃, (m₂^{7,2′-O})Gppp(m^{2′-O})N₁pN₂pN₃, (m₂^{7,3′-O})Gppp(m^{2′-O})N₁pN₂N₃), wherein N₁, N₂, and N₃ are as defined and described herein. In some embodiments, the 5′ cap is a tetranucleotide Cap2 structure (e.g., (m⁷)Gppp(m^{2′-O})N₁p(m^{2′-O})N₂pN₃, (m₂^{7,2′-O})Gppp(m^{2′-O})N₁p(m^{2′-O})N₂pN₃, (m₂^{7,3′-O})Gppp(m^{2′-O})N₁p(m^{2′-O})N₂pN₃), wherein N₁, N₂, and N₃ are as defined and described herein. In some embodiments, the 5′ cap is selected from the group consisting of (m₂^{7,3′-O})Gppp(m^{2′-O})Ap(m^{2′-O})GpG, (m₂^{7,3′-O})Gppp(m^{2′-O})Gp(m^{2′-O})GpC, (m⁷)Gppp(m^{2′-O})Ap(m^{2′-O})UpA, and (m⁷)Gppp(m^{2′-O})Ap(m^{2′-O})GpG.

In some embodiments, the 5′ cap is (m₂^{7,3′-O})Gppp(m^{2′-O})Ap(m^{2′-O})GpG, having a structure:

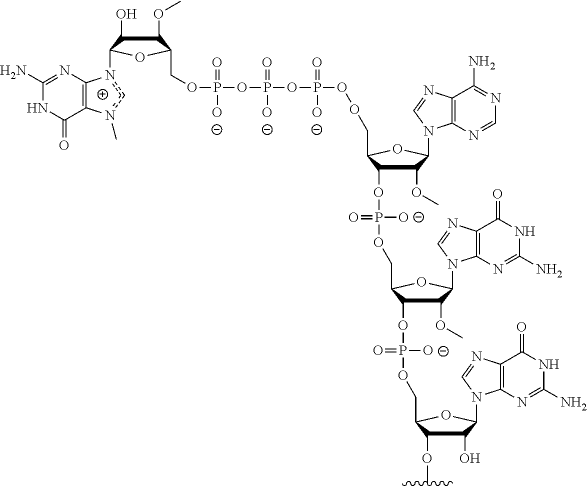

or a salt thereof.

In some embodiments, the 5′ cap is (m₂^{7,3′-O})Gppp(m^{2′-O})Gp(m^{2′-O})GpC, having a structure:

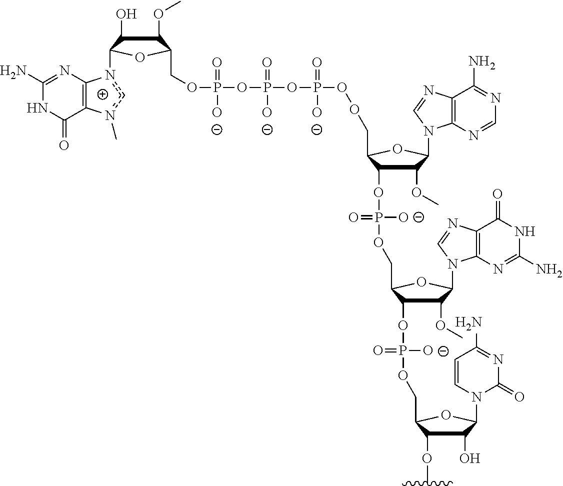

or a salt thereof.

In some embodiments, the 5′ cap is (m⁷)Gppp(m^{2′-O})Ap(m^{2′-O})UpA, having a structure:

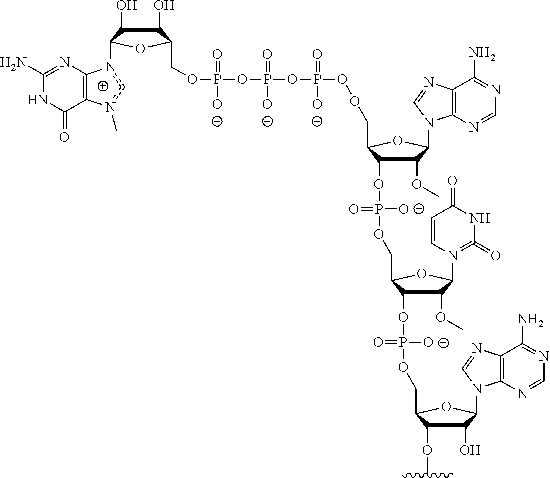

or a salt thereof.

In some embodiments, the 5′ cap is (m⁷)Gppp(m^{2′-O})Ap(m^{2′-O})GpG, having a structure:

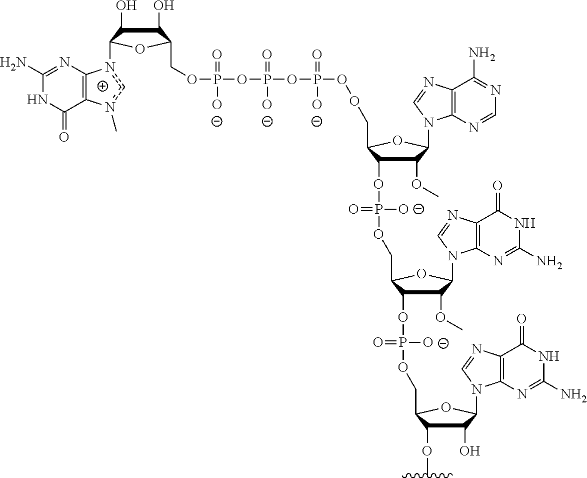

or a salt thereof.

### 2. Cap Proximal Sequences

In some embodiments, a 5′ UTR utilized in accordance with the present disclosure comprises a cap proximal sequence, e.g., as disclosed herein. In some embodiments, a cap proximal sequence comprises a sequence adjacent to a 5′ cap. In some embodiments, a cap proximal sequence comprises nucleotides in positions +1, +2, +3, +4, and/or +5 of an RNA polynucleotide.

In some embodiments, a cap structure comprises one or more polynucleotides of a cap proximal sequence. In some embodiments, a cap structure comprises an m⁷ Guanosine cap and nucleotide +1 (N₁) of an RNA polynucleotide. In some embodiments, a cap structure comprises an m⁷ Guanosine cap and nucleotide +2 (N₂) of an RNA polynucleotide. In some embodiments, a cap structure comprises an m⁷ Guanosine cap and nucleotides +1 and +2 (N₁ and N₂) of an RNA polynucleotide. In some embodiments, a cap structure comprises an m⁷ Guanosine cap and nucleotides +1, +2, and +3 (N₁, N₂, and N₃) of an RNA polynucleotide.

Those skilled in the art, reading the present disclosure, will appreciate that, in some embodiments, one or more residues of a cap proximal sequence (e.g., one or more of residues +1, +2, +3, +4, and/or +5) may be included in an RNA by virtue of having been included in a cap entity (e.g., a cap1 or cap2 structure, etc.); alternatively, in some embodiments, at least some of the residues in a cap proximal sequence may be enzymatically added (e.g., by a polymerase such as a T7 polymerase). For example, in certain exemplified embodiments where a m₂^{7,3′-O}Gppp(m₁^{2′-O})ApG cap is utilized, +1 (i.e., N₁) and +2 (i.e. N₂) are the (m₁^{2′-O})A and G residues of the cap, and +3, +4, and +5 are added by polymerase (e.g., T7 polymerase).

In some embodiments, the 5′ cap is a dinucleotide cap structure, wherein the cap proximal sequence comprises N₁ of the 5′ cap, where N₁ is any nucleotide, e.g., A, C, G or U. In some embodiments, the 5′ cap is a trinucleotide cap structure (e.g., the trinucleotide cap structures described above and herein), wherein the cap proximal sequence comprises N₁ and N₂ of the 5′ cap, wherein N₁ and N₂ are independently any nucleotide, e.g., A, C, G or U. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁, N₂, and N₃ are any nucleotide, e.g., A, C, G or U.

In some embodiments, e.g., where the 5′ cap is a dinucleotide cap structure, a cap proximal sequence comprises N₁ of a the 5′ cap, and N₂, N₃, N₄ and N₅, wherein N₁ to N₅ correspond to positions +1, +2, +3, +4, and/or +5 of an RNA polynucleotide. In some embodiments, e.g., where the 5′ cap is a trinucleotide cap structure, a cap proximal sequence comprises N₁ and N₂ of a the 5′ cap, and N₃, N₄ and N₅, wherein N₁ to N₅ correspond to positions +1, +2, +3, +4, and/or +5 of an RNA polynucleotide. In some embodiments, e.g., where the 5′ cap is a tetranucleotide cap structure, a cap proximal sequence comprises N₁, N₂, and N₃ of a the 5′ cap, and N₄ and N₅, wherein N₁ to N₅ correspond to positions +1, +2, +3, +4, and/or +5 of an RNA polynucleotide.

In some embodiments, N₁ is A. In some embodiments, N₁ is C. In some embodiments, N₁ is G. In some embodiments, N₁ is U. In some embodiments, N₂ is A. In some embodiments, N₂ is C. In some embodiments, N₂ is G. In some embodiments, N₂ is U. In some embodiments, N₃ is A. In some embodiments, N₃ is C. In some embodiments, N₃ is G. In some embodiments, N₃ is U. In some embodiments, N₄ is A. In some embodiments, N₄ is C. In some embodiments, N₄ is G. In some embodiments, N₄ is U. In some embodiments, N₅ is A. In some embodiments, N₅ is C. In some embodiments, N₅ is G. In some embodiments, N₅ is U. It will be understood that, each of the embodiments described above and herein (e.g., for N₁ through N₅) may be taken singly or in combination and/or may be combined with other embodiments of variables described above and herein (e.g., 5′ caps).

In some embodiments, a cap proximal sequence comprises A₁ and G₂ of the Cap1 structure, and a sequence comprising: A₃A₄U₅ (SEQ ID NO: 207) at positions +3, +4 and +5 respectively of the polyribonucleotide.

### 3. 5′ UTR

In some embodiments, a nucleic acid (e.g., DNA, RNA) utilized in accordance with the present disclosure comprises a 5′-UTR. In some embodiments, 5′-UTR may comprise a plurality of distinct sequence elements; in some embodiments, such plurality may be or comprise multiple copies of one or more particular sequence elements (e.g., as may be from a particular source or otherwise known as a functional or characteristic sequence element). In some embodiments a 5′ UTR comprises multiple different sequence elements.

The term “untranslated region” or “UTR” is commonly used in the art to a region in a DNA molecule which is transcribed but is not translated into an amino acid sequence, or to the corresponding region in an RNA polynucleotide, such as an mRNA molecule. An untranslated region (UTR) can be present 5′ (upstream) of an open reading frame (5′-UTR) and/or 3′ (downstream) of an open reading frame (3′-UTR). As used herein, the terms “five prime untranslated region” or “5′ UTR” refer to a sequence of a polyribonucleotide between the 5′ end of the polyribonucleotide (e.g., a transcription start site) and a start codon of a coding region of the polyribonucleotide. In some embodiments, “5′ UTR” refers to a sequence of a polyribonucleotide that begins at the 5′ end of the polyribonucleotide (e.g., a transcription start site) and ends one nucleotide (nt) before a start codon (usually AUG) of a coding region of the polyribonucleotide, e.g., in its natural context. In some embodiments, a 5′ UTR comprises a Kozak sequence. A 5′-UTR is downstream of the 5′-cap (if present), e.g., directly adjacent to the 5′-cap. In some embodiments, a 5′ UTR disclosed herein comprises a cap proximal sequence, e.g., as defined and described herein. In some embodiments, a cap proximal sequence comprises a sequence adjacent to a 5′ cap.

Exemplary 5′ UTRs include a human alpha globin (hAg) 5′UTR or a fragment thereof, a TEV 5′ UTR or a fragment thereof, a HSP70 5′ UTR or a fragment thereof, or a c-Jun 5′ UTR or a fragment thereof. In some embodiments, an RNA disclosed herein comprises a hAg 5′ UTR or a fragment thereof.

In some embodiments, an RNA disclosed herein comprises a 5′ UTR having at least 80%, at least 85%, at least 90%, at least 95%, at least 96%, at least 97%, at least 98%, or at least 99% identity to a 5′ UTR with the sequence AGAATAAACTAGTATTCTTCTGGTCCCCACAGACTCAGAGAGAACCCGCCACC (SEQ ID NO: 208). In some embodiments, an RNA disclosed herein comprises a 5′ UTR having the sequence

(SEQ ID NO: 208)
AGAATAAACTAGTATTCTTCTGGTCCCCACAGACTCAGAGAGAACCCGCC
ACC.

In some embodiments, an RNA disclosed herein comprises a 5′ UTR having at least 80%, at least 85%, at least 90%, at least 95%, at least 96%, at least 97%, at least 98%, or at least 99% identity to a 5′ UTR with the sequence AACUAGUAUUCUUCUGGUCCCCACAGACUCAGAGAGAACCCGCCACC (SEQ ID NO: 209)(hAg-Kozak/5′UTR). In some embodiments, an RNA disclosed herein comprises a 5′ UTR having the sequence

(SEQ ID NO: 209)
AACUAGUAUUCUUCUGGUCCCCACAGACUCAGAGAGAACCCGCCACC
(hAg-Kozak/5′UTR).

### 4. PolyA Tail

In some embodiments, a polynucleotide (e.g., DNA, RNA) disclosed herein comprises a polyadenylate (polyA) sequence, e.g., as described herein. In some embodiments, a polyA sequence is situated downstream of a 3′-UTR, e.g., adjacent to a 3′-UTR.

As used herein, the term “poly(A) sequence” or “poly-A tail” refers to an uninterrupted or interrupted sequence of adenylate residues which is typically located at the 3′-end of an RNA polynucleotide. Poly(A) sequences are known to those of skill in the art and may follow the 3′-UTR in the RNAs described herein. An uninterrupted poly(A) sequence is characterized by consecutive adenylate residues. In nature, an uninterrupted poly(A) sequence is typical. In some embodiments, polynucleotides disclosed herein comprise an uninterrupted Poly(A) sequence. In some embodiments, polynucleotides disclosed herein comprise interrupted Poly(A) sequence. In some embodiments, RNAs disclosed herein can have a poly(A) sequence attached to the free 3-end of the RNA by a template-independent RNA polymerase after transcription or a poly(A) sequence encoded by DNA and transcribed by a template-dependent RNA polymerase.

It has been demonstrated that a poly(A) sequence of about 120 A nucleotides has a beneficial influence on the levels of RNA in transfected eukaryotic cells, as well as on the levels of protein that is translated from an open reading frame that is present upstream (5′) of the poly(A) sequence (Holtkamp et al., 2006, Blood, vol. 108, pp. 4009-4017, which is herein incorporated by reference).

In some embodiments, a poly(A) sequence in accordance with the present disclosure is not limited to a particular length; in some embodiments, a poly(A) sequence is any length. In some embodiments, a poly(A) sequence comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly(A) sequence, typically at least 75%, at least 80%, at least 85%, at least 90%, at least 95%, at least 96%, at least 97%, at least 98%, or at least 99% by number of nucleotides in the poly(A) sequence are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly(A) sequence, i.e., 100% by number of nucleotides in the poly(A) sequence, are A nucleotides. The term “A nucleotide” or “A” refers to adenylate.

In some embodiments, a poly(A) sequence is attached during RNA transcription, e.g., during preparation of in vitro transcribed RNA, based on a DNA template comprising repeated dT nucleotides (deoxythymidylate) in the strand complementary to the coding strand. The DNA sequence encoding a poly(A) sequence (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1, which is herein incorporated by reference may be used in accordance with the present disclosure. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. In some embodiments, the poly(A) sequence contained in an RNA polynucleotide described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly(A) sequence at its 3-end, i.e., the poly(A) sequence is not masked or followed at its 3-end by a nucleotide other than A.

In some embodiments, the poly(A) sequence may comprise at least 20, at least 30, at least 40, at least 80, or at least 100 and up to 500, up to 400, up to 300, up to 200, or up to 150 nucleotides. In some embodiments, the poly(A) sequence may essentially consist of at least 20, at least 30, at least 40, at least 80, or at least 100 and up to 500, up to 400, up to 300, up to 200, or up to 150 nucleotides. In some embodiments, the poly(A) sequence may consist of at least 20, at least 30, at least 40, at least 80, or at least 100 and up to 500, up to 400, up to 300, up to 200, or up to 150 nucleotides. In some embodiments, the poly(A) sequence comprises at least 100 nucleotides. In some embodiments, the poly(A) sequence comprises about 150 nucleotides. In some embodiments, the poly(A) sequence comprises about 120 nucleotides.

In some embodiments, a poly A tail comprises a specific number of Adenosines, such as about 50 or more, about 60 or more, about 70 or more, about 80 or more, about 90 or more, about 100 or more, about 120, or about 150 or about 200. In some embodiments a poly A tail of a string construct may comprise 200 A residues or less. In some embodiments, a poly A tail of a string construct may comprise about 200 A residues. In some embodiments, a poly A tail of a string construct may comprise 180 A residues or less. In some embodiments, a poly A tail of a string construct may comprise about 180 A residues. In some embodiments, a poly A tail may comprise 150 residues or less.

In some embodiments, RNA comprises a poly(A) sequence comprising the nucleotide sequence of AAAAAAAAAAAAAAAAAAAAAAAAAAAAAAGCATATGACTAAAAAAAAAAAA AAAAAAAAAAAAAAAAAAAAAAAAAAAAAAAAAAAAAAAAAAAAAAAAAAA AAAAAAA (SEQ ID NO: 210), or a nucleotide sequence having at least 99%, 98%, 97%, 96%, 95%, 90%, 85%, or 80% identity to the nucleotide sequence of AAAAAAAAAAAAAAAAAAAAAAAAAAAAAAGCATATGACTAAAAAAAAAAAA AAAAAAAAAAAAAAAAAAAAAAAAAAAAAAAAAAAAAAAAAAAAAAAAAAA AAAAAAA (SEQ ID NO: 210). In some embodiments, a poly(A) tail comprises a plurality of A residues interrupted by a linker. In some embodiments, a linker comprises the nucleotide sequence GCATATGAC (SEQ ID NO: 211).

In some embodiments, RNA comprises a poly(A) sequence comprising the nucleotide sequence of AAAAAAAAAAAAAAAAAAAAAAAAAAAAAAGCAUAUGACUAAAAAAAAAAA AAAAAAAAAAAAAAAAAAAAAAAAAAAAAAAAAAAAAAAAAAAAAAAAAAA AAAAAAAA (SEQ ID NO: 212), or a nucleotide sequence having at least 99%, 98%, 97%, 96%, 95%, 90%, 85%, or 80% identity to the nucleotide sequence of AAAAAAAAAAAAAAAAAAAAAAAAAAAAAAGCAUAUGACUAAAAAAAAAAA AAAAAAAAAAAAAAAAAAAAAAAAAAAAAAAAAAAAAAAAAAAAAAAAAAA AAAAAAAA (SEQ ID NO: 213). In some embodiments, a poly(A) tail comprises a plurality of A residues interrupted by a linker. In some embodiments, a linker comprises the nucleotide sequence GCAUAUGAC (SEQ ID NO: 214).

### 5. 3′ UTR

In some embodiments, an RNA utilized in accordance with the present disclosure comprises a 3′-UTR.

As used herein, the terms “three prime untranslated region,” “3′ untranslated region,” or “3′ UTR” refer to a sequence of an mRNA molecule that begins following a stop codon of a coding region of an open reading frame sequence. In some embodiments, the 3′ UTR begins immediately after a stop codon of a coding region of an open reading frame sequence, e.g., in its natural context. In other embodiments, the 3′ UTR does not begin immediately after stop codon of the coding region of an open reading frame sequence, e.g., in its natural context. The term “3′-UTR” does preferably not include the poly(A) sequence. Thus, the 3′-UTR is upstream of the poly(A) sequence (if present), e.g. directly adjacent to the poly(A) sequence.

In some embodiments, an RNA disclosed herein comprises a 3′ UTR comprising an F element and/or an I element. In some embodiments, a 3′ UTR or a proximal sequence thereto comprises a restriction site. In some embodiments, a restriction site is a BamHI site. In some embodiments, a restriction site is a XhoI site.

In some embodiments, an RNA construct comprises an F element. In some embodiments, an F element sequence is a 3′-UTR of amino-terminal enhancer of split (AES).

In some embodiments, an RNA disclosed herein comprises a 3′ UTR having at least 80%, at least 85%, at least 90%, at least 95%, at least 96%, at least 97%, at least 98%, or at least 99% identity to a 3′ UTR with the sequence of CTGGTACTGCATGCACGCAATGCTAGCTGCCCCTTTCCCGTCCTGGGTACCCCGA GTCTCCCCCGACCTCGGGTCCCAGGTATGCTCCCACCTCCACCTGCCCCACTCAC CACCTCTGCTAGTTCCAGACACCTCCCAAGCACGCAGCAATGCAGCTCAAAACG CTTAGCCTAGCCACACCCCCACGGGAAACAGCAGTGATTAACCTTTAGCAATAA ACGAAAGTTTAACTAAGCTATACTAACCCCAGGGTTGGTCAATTTCGTGCCAGC CACACC (SEQ ID NO: 215). In some embodiments, an RNA disclosed herein comprises a 3′ UTR with the sequence of

(SEQ ID NO: 215)
CTGGTACTGCATGCACGCAATGCTAGCTGCCCCTTTCCCGTCCTGGGTAC
CCCGAGTCTCCCCCGACCTCGGGTCCCAGGTATGCTCCCACCTCCACCTG
CCCCACTCACCACCTCTGCTAGTTCCAGACACCTCCCAAGCACGCAGCAA
TGCAGCTCAAAACGCTTAGCCTAGCCACACCCCCACGGGAAACAGCAGTG
ATTAACCTTTAGCAATAAACGAAAGTTTAACTAAGCTATACTAACCCCAG
GGTTGGTCAATTTCGTGCCAGCCACACC.

In some embodiments, an RNA disclosed herein comprises a 3′ UTR provided in SEQ ID NO: 215.

In some embodiments, an RNA disclosed herein comprises a 3′ UTR having at least 80%, at least 85%, at least 90%, at least 95%, at least 96%, at least 97%, at least 98%, or at least 99% identity to a 3′ UTR with the sequence of CUGGUACUGCAUGCACGCAAUGCUAGCUGCCCCUUUCCCGUCCUGGGUACCCC GAGUCUCCCCCGACCUCGGGUCCCAGGUAUGCUCCCACCUCCACCUGCCCCAC UCACCACCUCUGCUAGUUCCAGACACCUCCCAAGCACGCAGCAAUGCAGCUCA AAACGCUUAGCCUAGCCACACCCCCACGGGAAACAGCAGUGAUUAACCUUUA GCAAUAAACGAAAGUUUAACUAAGCUAUACUAACCCCAGGGUUGGUCAAUUU CGUGCCAGCCACACC (SEQ ID NO: 216). In some embodiments, an RNA disclosed herein comprises a 3′ UTR with the sequence of

(SEQ ID NO: 216)
CUGGUACUGCAUGCACGCAAUGCUAGCUGCCCCUUUCCCGUCCUGGGUAC
CCCGAGUCUCCCCCGACCUCGGGUCCCAGGUAUGCUCCCACCUCCACCUG
CCCCACUCACCACCUCUGCUAGUUCCAGACACCUCCCAAGCACGCAGCAA
UGCAGCUCAAAACGCUUAGCCUAGCCACACCCCCACGGGAAACAGCAGUG
AUUAACCUUUAGCAAUAAACGAAAGUUUAACUAAGCUAUACUAACCCCAG
GGUUGGUCAAUUUCGUGCCAGCCACACC.

In some embodiments, an RNA disclosed herein comprises a 3′ UTR provided in SEQ ID NO: 216.

In some embodiments, a 3′UTR is an FI element as described in WO2017/060314, which is herein incorporated by reference in its entirety.

### B. RNA Formats

At least three distinct formats useful for RNA compositions (e.g., pharmaceutical compositions) have been developed, namely non-modified uridine containing mRNA (uRNA), nucleoside-modified mRNA (modRNA), and self-amplifying mRNA (saRNA). Each of these platforms displays unique features. Each of these platforms displays unique features. In general, in all three formats, RNA is capped, contains open reading frames (ORFs) flanked by untranslated regions (UTR), and have a polyA-tail at the 3′ end. An ORF of an uRNA and modRNA vectors encode an antibody agent or fragment thereof. A saRNA has multiple ORFs.

In some embodiments, the RNA described herein may have modified nucleosides. In some embodiments, the RNA comprises a modified nucleoside in place of at least one (e.g., every) uridine.

The term “uracil,” as used herein, describes one of the nucleobases that can occur in the nucleic acid of RNA. The structure of uracil is:

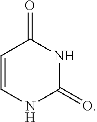

The term “uridine,” as used herein, describes one of the nucleosides that can occur in RNA. The structure of uridine is:

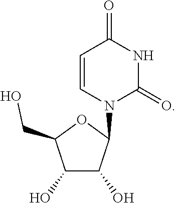 [SMILES: O=c1ccn([C@@H]2O[C@H](CO)[C@@H](O)[C@H]2O)c(=O)[nH]1]

UTP (uridine 5′-triphosphate) has the following structure:

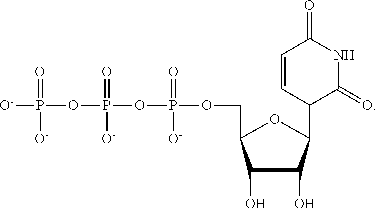

Pseudo-UTP (pseudouridine 5′-triphosphate) has the following structure:

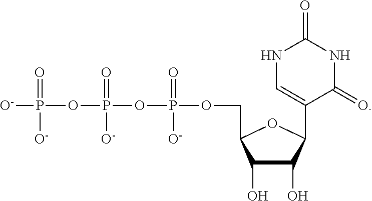

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N1-methyl-pseudouridine (m1Ψ), which has the structure:

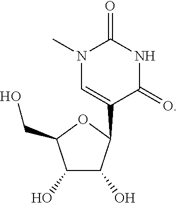 [SMILES: Cn1cc([C@@H]2O[C@H](CO)[C@@H](O)[C@H]2O)c(=O)[nH]c1=O]

N1-methyl-pseudo-UTP has the following structure:

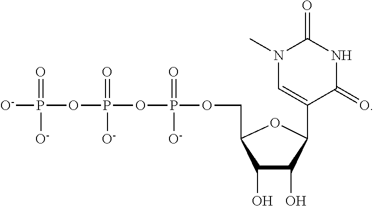

Another exemplary modified nucleoside is 5-methyl-uridine (m5U), which has the structure:

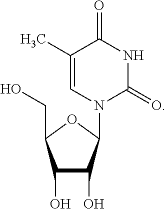

In some embodiments, one or more uridine in an RNA described herein is replaced by a modified nucleoside. In some embodiments, the modified nucleoside is a modified uridine.

In some embodiments, RNA comprises a modified nucleoside in place of at least one uridine. In some embodiments, RNA comprises a modified nucleoside in place of each uridine.

In some embodiments, the modified nucleoside is independently selected from pseudouridine (ψ), N₁-methyl-pseudouridine (m1ψ), and 5-methyl-uridine (m5U). In some embodiments, the modified nucleoside comprises pseudouridine (ψ). In some embodiments, the modified nucleoside comprises N₁-methyl-pseudouridine (m1ψ). In some embodiments, the modified nucleoside comprises 5-methyl-uridine (m5U). In some embodiments, RNA may comprise more than one type of modified nucleoside, and the modified nucleosides are independently selected from pseudouridine (W), N₁-methyl-pseudouridine (m1ψ), and 5-methyl-uridine (m5U). In some embodiments, the modified nucleosides comprise pseudouridine (ψ) and N₁-methyl-pseudouridine (m1ψ). In some embodiments, the modified nucleosides comprise pseudouridine (W) and 5-methyl-uridine (m5U). In some embodiments, the modified nucleosides comprise N1-methyl-pseudouridine (m1ψ) and 5-methyl-uridine (m5U). In some embodiments, the modified nucleosides comprise pseudouridine (ψ), N1-methyl-pseudouridine (m1ψ), and 5-methyl-uridine (m5U).

In some embodiments, the modified nucleoside replacing one or more, e.g., all, uridine in the RNA may be any one or more of 3-methyl-uridine (m3U), 5-methoxy-uridine (mo5U), 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridine or 5-bromo-uridine), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 1-ethyl-pseudouridine, 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (τm5U), 1-taurinomethyl-pseudouridine, 5-taurinomethyl-2-thio-uridine(τm5s2U), 1-taurinomethyl-4-thio-pseudouridine), 5-methyl-2-thio-uridine (m5s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N₁-methyl-pseudouridine, 3-(3-amino-3-carboxypropyl)uridine (acp3U), 1-methyl-3-(3-amino-3-carboxypropyl)pseudouridine (acp3 ψ), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-0-dimethyl-uridine (m3Um), 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

In some embodiments, the RNA comprises other modified nucleosides or comprises further modified nucleosides, e.g., modified cytidine. For example, in some embodiments, in the RNA 5-methylcytidine is substituted partially or completely, preferably completely, for cytidine. In some embodiments, the RNA comprises 5-methylcytidine and one or more selected from pseudouridine (ψ), N1-methyl-pseudouridine (m1ψ), and 5-methyl-uridine (m5U). In some embodiments, the RNA comprises 5-methylcytidine and N1-methyl-pseudouridine (m1ψ). In some embodiments, the RNA comprises 5-methylcytidine in place of each cytidine and N1-methyl-pseudouridine (m1ψ) in place of each uridine.

In some embodiments of the present disclosure, the RNA is “replicon RNA” or simply a “replicon,” in particular “self-replicating RNA” or “self-amplifying RNA.” In one particularly preferred embodiment, the replicon or self-replicating RNA is derived from or comprises elements derived from a single-stranded (ss) RNA virus, in particular a positive-stranded ssRNA virus, such as an alphavirus. Alphaviruses are typical representatives of positive-stranded RNA viruses. Alphaviruses replicate in the cytoplasm of infected cells (for review of the alphaviral life cycle see Jose et al., Future Microbiol., 2009, vol. 4, pp. 837-856, which is incorporated herein by reference in its entirety). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124, which is incorporated herein by reference in its entirety). Following infection, i.e. at early stages of the viral life cycle, the (+) stranded genomic RNA directly acts like a messenger RNA for the translation of the open reading frame encoding the non-structural poly-protein (nsP1234).

Alphavirus-derived vectors have been proposed for delivery of foreign genetic information into target cells or target organisms. In simple approaches, a first ORF encodes an alphavirus-derived RNA-dependent RNA polymerase (replicase), which upon translation mediates self-amplification of the RNA. A second ORF encoding alphaviral structural proteins is replaced by an open reading frame encoding a HSV (HSV-1 and/or HSV-2) construct described herein. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

Features of a non-modified uridine platform may include, for example, one or more of intrinsic adjuvant effect, as well as good tolerability and safety. Features of modified uridine (e.g., pseudouridine) platform may include reduced adjuvant effect, blunted immune innate immune sensor activating capacity and thus good tolerability and safety. Features of self-amplifying platform may include, for example, long duration of protein expression, good tolerability and safety, higher likelihood for efficacy with very low vaccine dose.

The present disclosure provides particular RNA constructs optimized, for example, for improved manufacturability, encapsulation, expression level (and/or timing), etc. Certain components are discussed below, and certain preferred embodiments are exemplified herein.

### C. Codon Optimization and GC Enrichment

As used herein, the term “codon-optimized” refers to alteration of codons in a coding region of a nucleic acid molecule (e.g., a polyribonucleotide) to reflect the typical codon usage of a host organism (e.g., a subject receiving a nucleic acid molecule (e.g., a polyribonucleotide)) without preferably altering the amino acid sequence encoded by the nucleic acid molecule. Within the context of the present disclosure, in some embodiments, coding regions are codon-optimized for optimal expression in a subject to be treated using the RNA molecules described herein. In some embodiments, codon-optimization may be performed such that codons for which frequently occurring tRNAs are available are inserted in place of “rare codons.” In some embodiments, codon-optimization may include increasing guanosine/cytosine (G/C) content of a coding region of RNA described herein as compared to the G/C content of the corresponding coding sequence of a wild type RNA, wherein the amino acid sequence encoded by the RNA is preferably not modified compared to the amino acid sequence.

In some embodiments, a coding sequence (also referred to as a “coding region”) is codon optimized for expression in the subject to whom a composition (e.g., a pharmaceutical composition) is to be administered (e.g., a human). Thus, in some embodiments, sequences in such a polynucleotide (e.g., a polyribonucleotide) may differ from wild type sequences encoding the relevant antigen or antigenic fragment or epitope thereof, even when the amino acid sequence of the antigen or antigenic fragment or epitope thereof is wild type.

In some embodiments, strategies for codon optimization for expression in a relevant subject (e.g., a human), and even, in some cases, for expression in a particular cell or tissue.

In general, as is understood, codon optimization refers to a process of modifying a nucleic acid sequence for enhanced expression in a subject or its cells of interest by replacing at least one codon (e.g., about or more than about 1, 2, 3, 4, 5, 10, 15, 20, 25, 50, or more codons) of a native sequence with codons that are more frequently or most frequently used in the genes of that subject or its cells while maintaining the native amino acid sequence.

Various species exhibit particular bias for certain codons of a particular amino acid. Without wishing to be bound by any one theory, codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell may generally be a reflection of the codons used most frequently in peptide synthesis. Accordingly, genes may be tailored for optimal gene expression in a given organism based on codon optimization. Codon usage tables are available, for example, at the “Codon Usage Database” available at www.kazusa.orjp/codon/and these tables may be adapted in a number of ways. Computer algorithms for codon optimizing a particular sequence for expression in a particular subject or its cells are also available, such as Gene Forge (Aptagen; Jacobus, PA), are also available.

In some embodiments, a polynucleotide (e.g., a polyribonucleotide) of the present disclosure is codon optimized, wherein the codons in the polynucleotide (e.g., the polyribonucleotide) are adapted to human codon usage (herein referred to as “human codon optimized polynucleotide”). Codons encoding the same amino acid occur at different frequencies in a subject, e.g., a human. Accordingly, in some embodiments, the coding sequence of a polynucleotide of the present disclosure is modified such that the frequency of the codons encoding the same amino acid corresponds to the naturally occurring frequency of that codon according to the human codon usage, e.g., as shown in Table 12. For example, in the case of the amino acid Ala, the wild type coding sequence is preferably adapted in a way that the codon “GCC” is used with a frequency of 0.40, the codon “GCT” is used with a frequency of 0.28, the codon “GCA” is used with a frequency of 0.22 and the codon “GCG” is used with 30 a frequency of 0.10 etc. (see Table 12). Accordingly, in some embodiments, such a procedure (as exemplified for Ala) is applied for each amino acid encoded by the coding sequence of a polynucleotide to obtain sequences adapted to human codon usage.

TABLE 13
Human codon usage with frequencies indicated for each amino acid

| Amino acid | codon | frequency | Amino acid | codon | frequency |
|---|---|---|---|---|---|
| Ala | GCG | 0.10 | Pro | CCG | 0.11 |
| Ala | GCA | 0.22 | Pro | CCA | 0.27 |
| Ala | GCT | 0.28 | Pro | CCT | 0.29 |
| Ala | GCC* | 0.40 | Pro | CCC* | 0.33 |
| Cys | TGT | 0.42 | Gln | CAG* | 0.73 |
| Cys | TGC* | 0.58 | Gln | CAA | 0.27 |
| Asp | GAT | 0.44 | Arg | AGG | 0.22 |
| Asp | GAC* | 0.56 | Arg | AGA* | 0.21 |
| Glu | GAG* | 0.59 | Arg | CGG | 0.19 |
| Glu | GAA | 0.41 | Arg | CGA | 0.10 |
| Phe | TTT | 0.43 | Arg | CGT | 0.09 |
| Phe | TTC* | 0.57 | Arg | CGC | 0.19 |
| Gly | GGG | 0.23 | Ser | AGT | 0.14 |
| Gly | GGA | 0.26 | Ser | AGC* | 0.25 |
| Gly | GGT | 0.18 | Ser | TCG | 0.06 |
| Gly | GGC* | 0.33 | Ser | TCA | 0.15 |
| His | CAT | 0.41 | Ser | TCT | 0.18 |
| His | CAC* | 0.59 | Ser | TCC | 0.23 |
| Ile | ATA | 0.14 | Thr | ACG | 0.12 |
| Ile | ATT | 0.35 | Thr | ACA | 0.27 |
| Ile | ATC* | 0.52 | Thr | ACT | 0.23 |
| Lys | AAG* | 0.60 | Thr | ACC* | 0.38 |
| Lys | AAA | 0.40 | Val | GTG* | 0.48 |
| Leu | TTG | 0.12 | Val | GTA | 0.10 |
| Leu | TTA | 0.06 | Val | GTT | 0.17 |
| Leu | CTG* | 0.43 | Val | GTC | 0.25 |
| Leu | CTA | 0.07 | Trp | TGG* | 1 |
| Leu | CTT | 0.12 | Tyr | TAT | 0.42 |
| Leu | CTC | 0.20 | Tyr | TAC* | 0.58 |
| Met | ATG* | 1 | Stop | TGA* | 0.61 |
| Asn | AAT | 0.44 | Stop | TAG | 0.17 |
| Asn | AAC* | 0.56 | Stop | TAA | 0.22 |

Certain strategies for codon optimization and/or G/C enrichment for human expression are described in WO2002/098443, which is incorporated by reference herein in its entirety. In some embodiments, a coding sequence may be optimized using a multiparametric optimization strategy. In some embodiments, optimization parameters may include parameters that influence protein expression, which can be, for example, impacted on a transcription level, an mRNA level, and/or a translational level. In some embodiments, exemplary optimization parameters include, but are not limited to transcription-level parameters (including, e.g., GC content, consensus splice sites, cryptic splice sites, SD sequences, TATA boxes, termination signals, artificial recombination sites, and combinations thereof); mRNA-level parameters (including, e.g., RNA instability motifs, ribosomal entry sites, repetitive sequences, and combinations thereof); translation-level parameters (including, e.g., codon usage, premature poly(A) sites, ribosomal entry sites, secondary structures, and combinations thereof); or combinations thereof. In some embodiments, a coding sequence may be optimized by a GeneOptimizer algorithm as described in Fath et al. “Multiparameter RNA and Codon Optimization: A Standardized Tool to Assess and Enhance Autologous Mammalian Gene Expression” PLoS ONE 6(3): e17596; Rabb et al., which is incorporated herein by reference in its entirety, “The GeneOptimizer Algorithm: using a sliding window approach to cope with the vast sequence space in multiparameter DNA sequence optimization” Systems and Synthetic Biology (2010) 4:215-225; and Graft et al. “Codon-optimized genes that enable increased heterologous expression in mammalian cells and elicit efficient immune responses in mice after vaccination of naked DNA” Methods Mol Med (2004) 94:197-210, the entire content of each of which is incorporated herein for the purposes described herein. In some embodiments, a coding sequence may be optimized by Eurofins' adaption and optimization algorithm “GENEius” as described in Eurofins' Application Notes: Eurofins' adaption and optimization software “GENEius” in comparison to other optimization algorithms, the entire content of which is incorporated by reference for the purposes described herein.

In some embodiments, a coding sequence utilized in accordance with the present disclosure has G/C content that is increased compared to a coding sequence for an HSV (e.g., HSV-1 and/or HSV-2 polypeptide, or fragment thereof) construct described herein. In some embodiments, guanosine/cytidine (G/C) content of a coding region is modified relative to a comparable coding sequence for HSV (e.g., HSV-1 and/or HSV-2 polypeptide, or fragment thereof) construct described herein, but the amino acid sequence encoded by the polyribonucleotide not modified.

Without wishing to be bound by any particular theory, it is proposed that GC enrichment may improve translation of a payload sequence. Typically, sequences having an increased G (guanosine)/C (cytidine) content are more stable than sequences having an increased A (adenosine)/U (uridine) content. In respect to the fact that several codons code for one and the same amino acid (so-called degeneration of the genetic code), the most favorable codons for the stability can be determined (so-called alternative codon usage). Depending on the amino acid to be encoded by a polyribonucleotide, there are various possibilities for modification of the ribonucleic acid sequence, compared to its wild type sequence. In particular, codons which contain A and/or U nucleosides can be modified by substituting these codons by other codons, which code for the same amino acids but contain no A and/or U or contain a lower content of A and/or U nucleosides.

In some embodiments, G/C content of a coding region of a polyribonucleotide described herein is increased by at least 1%, at least 2%, at least 3%, at least 4%, at least 5%, at least 6%, or even more compared to the G/C content of the coding region prior to codon optimization, e.g., of the wild type RNA. In some embodiments, G/C content of a coding region of a polyribonucleotide described herein is decreased by at least 1%, at least 2%, at least 3%, at least 4%, at least 5%, at least 6%, or even more compared to the G/C content of the coding region prior to codon optimization, e.g., of the wild type RNA.

In some embodiments, stability and translation efficiency of a polyribonucleotide may incorporate one or more elements established to contribute to stability and/or translation efficiency of the polyribonucleotide; exemplary such elements are described, for example, in PCT/EP2006/009448 incorporated herein by reference. In some embodiments, to increase expression of a polyribonucleotide used according to the present disclosure, a polyribonucleotide may be modified within the coding region, i.e., the sequence encoding the expressed peptide or protein, without altering the sequence of the expressed peptide or protein, for example so as to increase the GC-content to increase mRNA stability and/or to perform a codon optimization and, thus, enhance translation in cells.

### IV. RNA Delivery Technologies

Provided polyribonucleotides may be delivered for therapeutic applications described herein using any appropriate methods known in the art, including, e.g., delivery as naked RNAs, or delivery mediated by viral and/or non-viral vectors, polymer-based vectors, lipid compositions, nanoparticles (e.g., lipid nanoparticles, polymeric nanoparticles, lipid-polymer hybrid nanoparticles, etc.), and/or peptide-based vectors. See, e.g., Wadhwa et al. “Opportunities and Challenges in the Delivery of mRNA-Based Vaccines” Pharmaceutics (2020) 102 (27 pages), the content of which is incorporated herein by reference, for information on various approaches that may be useful for delivery polyribonucleotides described herein.

In some embodiments, one or more polyribonucleotides can be formulated with lipid nanoparticles for delivery (e.g., administration).

In some embodiments, lipid nanoparticles can be designed to protect polyribonucleotides from extracellular RNases and/or engineered for systemic delivery of the RNA to target cells. In some embodiments, such lipid nanoparticles may be particularly useful to deliver polyribonucleotides when polyribonucleotides are intravenously or intramuscularly administered to a subject.

### A. Lipid Compositions

### 1. Lipids and Lipid-Like Materials

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of a polar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

Often, an amphiphilic compound has a polar head attached to a long hydrophobic tail. In some embodiments, the polar fragment is soluble in water, while the non-polar fragment is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

A “lipid-like material” is a substance that is structurally and/or functionally related to a lipid but may not be considered a lipid in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids.

Specific examples of amphiphilic compounds that may be included in an amphiphilic layer include, but are not limited to, phospholipids, aminolipids and sphingolipids.

Generally, lipids may be divided into eight categories: fatty acids, glycerolipids, glycerophospholipids, sphingolipids, saccharolipids, polyketides (derived from condensation of ketoacyl subunits), sterols and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as sterol-containing metabolites such as cholesterol.

Fatty acids are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

Glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are members of a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterols, such as cholesterol and its derivatives, or tocopherol and its derivatives, are important components of membrane lipids, along with the glycerophospholipids and sphingomyelins.

Saccharolipids are compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multinodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

Lipids and lipid-like materials may be cationic, anionic or neutral. Neutral lipids or lipid-like materials exist in an uncharged or neutral zwitterionic form at a selected pH.

In some embodiments, suitable lipids or lipid-like materials for use in the present disclosure include those described in WO2020/128031 and US20200163878, the entire contents of each of which are incorporated herein by reference for the purposes described herein.

### 2. Cationic or Cationically Ionizable Lipids or Lipid-Like Materials

In some embodiments cationic or cationically ionizable lipids or lipid-like materials contemplated for use herein include any cationic or cationically ionizable lipids or lipid-like materials which are able to electrostatically bind nucleic acid. In one embodiment, cationic or cationically ionizable lipids or lipid-like materials contemplated for use herein can be associated with nucleic acid, e.g. by forming complexes with the nucleic acid or forming vesicles in which the nucleic acid is enclosed or encapsulated.

Cationic lipids or lipid-like materials are characterized in that they have a net positive charge (e.g., at a relevant pH). Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In certain embodiments, a cationic lipid or lipid-like material has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

In some embodiments, a cationic or cationically ionizable lipid or lipid-like material comprises a head group which includes at least one nitrogen atom (N) which is positive charged or capable of being protonated.

Examples of cationic lipids include, but are not limited to 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (DAE-DMRIE), N-(4-carboxybenzyl)-N,N-dimethyl-2,3-bis(oleoyloxy)propan-1-aminium (DOBAQ), 2-({8-[(30)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N₁-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing. Further suitable cationic lipids for use in the present disclosure include those described in WO2020/128031 and US20200163878, the entire contents of each of which are incorporated herein by reference for the purposes described herein. Further suitable cationic lipids for use in the present disclosure include those described in WO2010/053572 (including Cl 2-200 described at paragraph [00225]) and WO2012/170930, both of which are incorporated herein by reference for the purposes described herein. Additional suitable cationic lipids for use in the present disclosure include HGT4003, HGT5000, HGTS001, HGT5001, HGT5002 (see US20150140070A1, which is incorporated herein by reference in its entirety).

In some embodiments, formulations that are useful for pharmaceutical compositions (e.g., immunogenic compositions, e.g., vaccines) compositions as described herein can comprise at least one cationic lipid. Representative cationic lipids include, but are not limited to, 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.CI), 1,2-dilinoleoyl-3-trimethylaminopropane chloride salt (DLin-TAP.CI), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); MC3 (US20100324120, which is incorporated herein by reference in its entirety).

In some embodiments, amino or cationic lipids useful in accordance with the present disclosure have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of lipids have to be present in the charged or neutral form. Lipids having more than one protonatable or deprotonatable group, or which are zwitterionic, are not excluded and may likewise suitable in the context of the present invention.

In some embodiments, a protonatable lipid has a pKa of the protonatable group in the range of about 4 to about 11, e.g., a pKa of about 5 to about 7.

In some embodiments, a cationic lipid may comprise from about 10 mol % to about 100 mol %, about 20 mol % to about 100 mol %, about 30 mol % to about 100 mol %, about 40 mol % to about 100 mol %, or about 50 mol % to about 100 mol % of total lipid present in a lipid composition utilized in accordance with the present disclosure.

### 3. Additional Lipids or Lipid-Like Materials

In some embodiments, formulations utilized in accordance with the present disclosure may comprise lipids or lipid-like materials other than cationic or cationically ionizable lipids or lipid-like materials, i.e., non-cationic lipids or lipid-like materials (including non-cationically ionizable lipids or lipid-like materials). Collectively, anionic and neutral lipids or lipid-like materials are referred to herein as non-cationic lipids or lipid-like materials. In some embodiments, optimizing a formulation of nucleic acid particles by addition of other hydrophobic moieties, such as cholesterol and lipids, in addition to an ionizable/cationic lipid or lipid-like material may, for example, enhance particle stability and efficacy of nucleic acid delivery.

In some embodiments, a lipid or lipid-like material may be incorporated which may or may not affect the overall charge of particles. In certain embodiments, such lipid or lipid-like material is a non-cationic lipid or lipid-like material.

In some embodiments, a non-cationic lipid may comprise, e.g., one or more anionic lipids and/or neutral lipids. An “anionic lipid” is negatively charged (e.g., at a selected pH).

A “neutral lipid” exists either in an uncharged or neutral zwitterionic form (e.g., at a selected pH). In some embodiments, a formulation comprises one of the following neutral lipid components: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Specific exemplary phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), and further phosphatidylethanolamine lipids with different hydrophobic chains.

In certain embodiments, a formulation utilized in accordance with the present disclosure includes DSPC or DSPC and cholesterol.

In certain embodiments, formulations utilized in accordance with the present disclosure include both a cationic lipid and an additional (non-cationic) lipid.

In some embodiments, formulations herein include a polymer conjugated lipid such as a pegylated lipid. “Pegylated lipids” comprise both a lipid portion and a polyethylene glycol portion. Pegylated lipids are known in the art.

Without wishing to be bound by theory, the amount of (total) cationic lipid compared to the amount of other lipid(s) in formulation may affect important characteristics, such as charge, particle size, stability, tissue selectivity, and bioactivity of the nucleic acid. In some embodiments, the molar ratio of the at least one cationic lipid to the at least one additional lipid is from about 10:0 to about 1:9, about 4:1 to about 1:2, or about 3:1 to about 1:1.

In some embodiments, a non-cationic lipid, in particular a neutral lipid, (e.g., one or more phospholipids and/or cholesterol) may comprise from about 0 mol % to about 90 mol %, from about 0 mol % to about 80 mol %, from about 0 mol % to about 70 mol %, from about 0 mol % to about 60 mol %, or from about 0 mol % to about 50 mol %, of the total lipid present in a formulation.

### 4. Lipoplex Particles

In certain embodiments of the present disclosure, the RNA described herein may be present in RNA lipoplex particles.

An “RNA lipoplex particle” contains lipid, in particular cationic lipid, and RNA. Electrostatic interactions between positively charged liposomes and negatively charged RNA results in complexation and spontaneous formation of RNA lipoplex particles. Positively charged liposomes may be generally synthesized using a cationic lipid, such as DOTMA, and additional lipids, such as DOPE. In one embodiment, a RNA lipoplex particle is a nanoparticle.

In certain embodiments, RNA lipoplex particles include both a cationic lipid and an additional lipid. In an exemplary embodiment, the cationic lipid is DOTMA and the additional lipid is DOPE.

In some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

In some embodiments, RNA lipoplex particles have an average diameter that in one embodiment ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, the RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm. In an embodiment, the RNA lipoplex particles have an average diameter that ranges from about 250 nm to about 700 nm. In another embodiment, the RNA lipoplex particles have an average diameter that ranges from about 300 nm to about 500 nm. In an exemplary embodiment, the RNA lipoplex particles have an average diameter of about 400 nm.

RNA lipoplex particles and compositions comprising RNA lipoplex particles described herein are useful for delivery of RNA to a target tissue after parenteral administration, in particular after intravenous administration. The RNA lipoplex particles may be prepared using liposomes that may be obtained by injecting a solution of the lipids in ethanol into water or a suitable aqueous phase. In one embodiment, the aqueous phase has an acidic pH. In one embodiment, the aqueous phase comprises acetic acid, e.g., in an amount of about 5 mM. Liposomes may be used for preparing RNA lipoplex particles by mixing the liposomes with RNA. In one embodiment, the liposomes and RNA lipoplex particles comprise at least one cationic lipid and at least one additional lipid. In one embodiment, the at least one cationic lipid comprises 1,2-di-O-octadecenyl-3-trimethylammonium propane (DOTMA) and/or 1,2-dioleoyl-3-trimethylammonium-propane (DOTAP). In one embodiment, the at least one additional lipid comprises 1,2-di-(9Z-octadecenoyl)-sn-glycero-3-phosphoethanolamine (DOPE), cholesterol (Chol) and/or 1,2-dioleoyl-sn-glycero-3-phosphocholine (DOPC). In one embodiment, the at least one cationic lipid comprises 1,2-di-O-octadecenyl-3-trimethylammonium propane (DOTMA) and the at least one additional lipid comprises 1,2-di-(9Z-octadecenoyl)-sn-glycero-3-phosphoethanolamine (DOPE). In one embodiment, the liposomes and RNA lipoplex particles comprise 1,2-di-O-octadecenyl-3-trimethylammonium propane (DOTMA) and 1,2-di-(9Z-octadecenoyl)-sn-glycero-3-phosphoethanolamine (DOPE).

Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In one embodiment, after administration of the RNA lipoplex particles, RNA accumulation and/or RNA expression in antigen presenting cells, such as professional antigen presenting cells in the spleen occurs. Thus, RNA lipoplex particles of the disclosure may be used for expressing RNA in such antigen presenting cells. In one embodiment, the antigen presenting cells are dendritic cells and/or macrophages.

### 5. Lipid Nanoparticles (LNPs)

In some embodiments, nucleic acid such as RNA described herein is administered in the form of lipid nanoparticles (LNPs). In some embodiments, LNPs may comprise any lipid capable of forming a particle to which the one or more nucleic acid molecules are attached, or in which the one or more nucleic acid molecules are encapsulated.

In some embodiments, an LNP comprises one or more cationic lipids, and one or more stabilizing lipids. Stabilizing lipids include neutral lipids and pegylated lipids.

In some embodiments, an LNP comprises a cationic lipid, a neutral lipid, a sterol, a polymer conjugated lipid; and an RNA, encapsulated within or associated with the lipid nanoparticle.

In some embodiments, a neutral lipid is selected from the group consisting of DSPC, DPPC, DMPC, DOPC, POPC, DOPE, DOPG, DPPG, POPE, DPPE, DMPE, DSPE, and SM. In some embodiments, the neutral lipid is selected from the group consisting of DSPC, DPPC, DMPC, DOPC, POPC, DOPE and SM. In some embodiments, the neutral lipid is DSPC.

In some embodiments, a sterol is cholesterol.

In some embodiments, a polymer conjugated lipid is a pegylated lipid. In some embodiments, a pegylated lipid has the following structure:

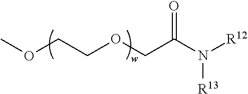

or a pharmaceutically acceptable salt, tautomer or stereoisomer thereof, wherein:
R¹² and R¹³ are each independently a straight or branched, saturated or unsaturated alkyl chain containing from 10 to 30 carbon atoms, wherein the alkyl chain is optionally interrupted by one or more ester bonds; and w has a mean value ranging from 30 to 60. In some embodiments, R¹² and R¹³ are each independently straight, saturated alkyl chains containing from 12 to 16 carbon atoms. In some embodiments, w has a mean value ranging from 40 to 55. In some embodiments, the average w is about 45. In some embodiments, R¹² and R¹³ are each independently a straight, saturated alkyl chain containing about 14 carbon atoms, and w has a mean value of about 45.

In some embodiments, a pegylated lipid is DMG-PEG 2000, e.g., having the following structure:

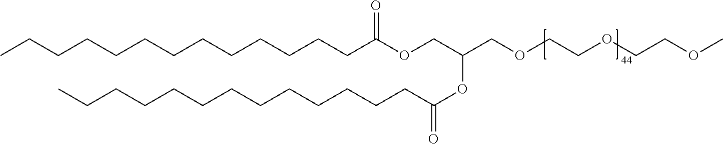

In some embodiments, a cationic lipid component of LNPs has the structure of Formula (III):

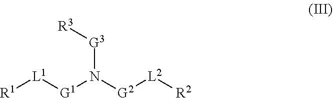

or a pharmaceutically acceptable salt, tautomer, prodrug or stereoisomer thereof, wherein: one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)X—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In some of the foregoing embodiments of Formula (III), the lipid has one of the following structures (IIIA) or (IIIB):

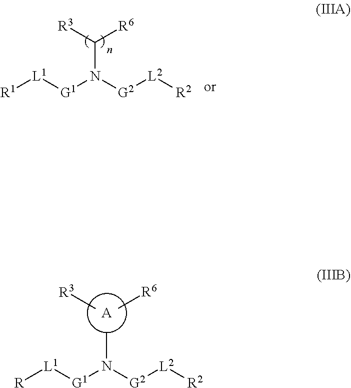

wherein:
A is a 3 to 8-membered cycloalkyl or cycloalkylene ring;
R⁶ is, at each occurrence, independently H, OH or C₁-C₂₄ alkyl; and
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

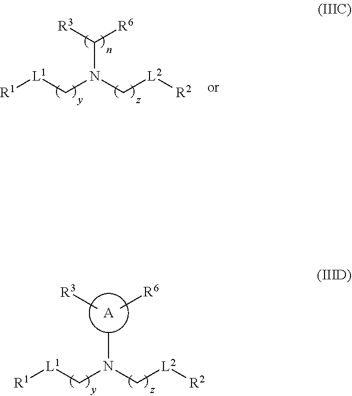

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—.

In some different embodiments of Formula (III), the lipid has one of the following structures (IIIE) or (IIIF):

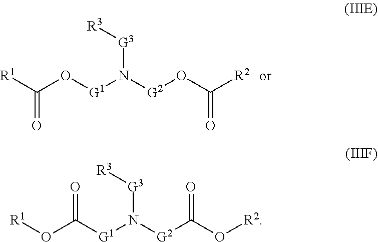

In some of the foregoing embodiments of Formula (III), the lipid has one of the following structures (IIIG), (IIIH), (IIII), or (IIIJ):

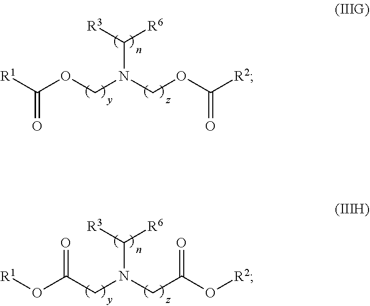

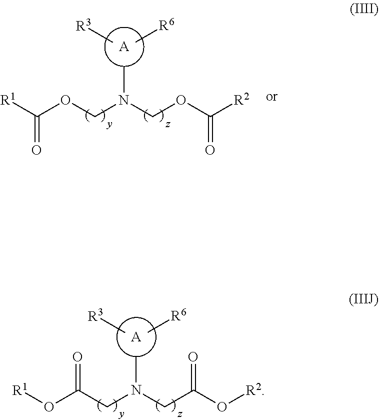

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G³ is unsubstituted. In other embodiments, G3 is substituted. In various different embodiments, G³ is linear C₁-C₂₄ alkylene or linear C₁-C₂₄ alkenylene.

In some other foregoing embodiments of Formula (III), R¹ or R², or both, is C₆-C₂₄ alkenyl. For example, in some embodiments, R¹ and R² each, independently have the following structure:

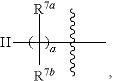

wherein:
R^{7a} and R^{7b} are, at each occurrence, independently H or C₁-C₁₂ alkyl; and a is an integer from 2 to 12, and
wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

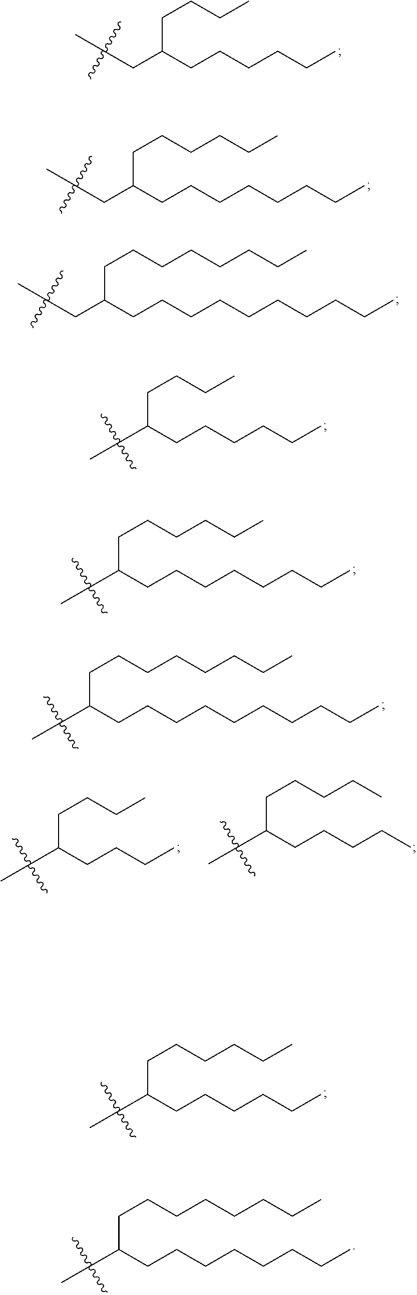

In some of the foregoing embodiments of Formula (III), R³ is OH, CN, —C(═O)OR⁴, —OC(═O)R⁴ or —NHC(═O)R⁴. In some embodiments, R⁴ is methyl or ethyl.

In various different embodiments, the cationic lipid of Formula (III) has one of the structures set forth in in Table 13 below.

TABLE 14
Exemplary Compounds of Formula (III).

| No. | Structure |
|---|---|
| III-1 | 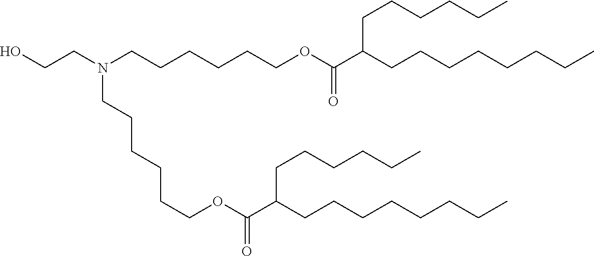 [SMILES: CCCCCCCCC(CCCCCC)C(=O)OCCCCCCN(CCO)CCCCCCOC(=O)C(CCCCCC)CCCCCCCC] |
| III-2 | 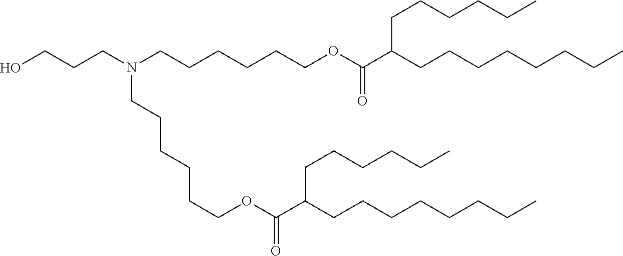 [SMILES: CCCCCCCCC(CCCCCC)C(=O)OCCCCCCN(CCCO)CCCCCCOC(=O)C(CCCCCC)CCCCCCCC] |
| III-3 | 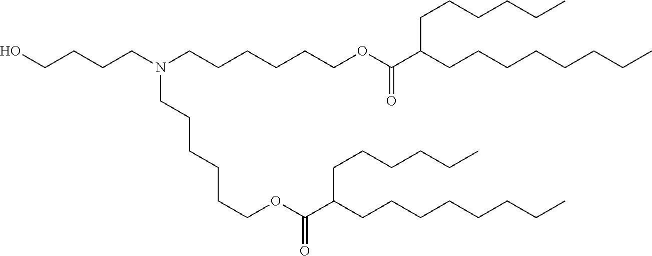 [SMILES: CCCCCCCCC(CCCCCC)C(=O)OCCCCCCN(CCCCO)CCCCCCOC(=O)C(CCCCCC)CCCCCCCC] |
| III-4 | 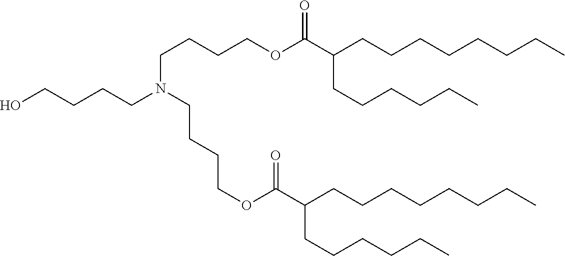 [SMILES: CCCCCCCCC(CCCCCC)C(=O)OCCCCN(CCCCO)CCCCOC(=O)C(CCCCCC)CCCCCCCC] |
| III-5 | 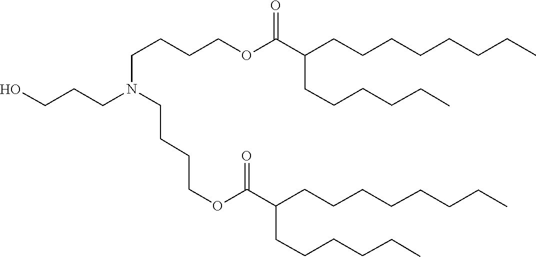 [SMILES: CCCCCCCCC(CCCCCC)C(=O)OCCCCN(CCCO)CCCCOC(=O)C(CCCCCC)CCCCCCCC] |
| III-6 | 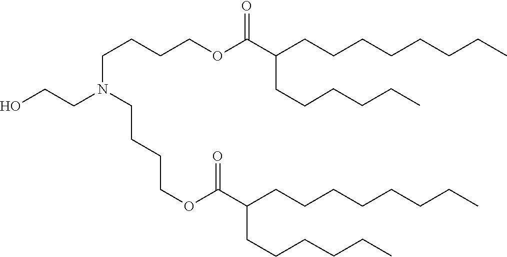 [SMILES: CCCCCCCCC(CCCCCC)C(=O)OCCCCN(CCO)CCCCOC(=O)C(CCCCCC)CCCCCCCC] |
| III-7 | 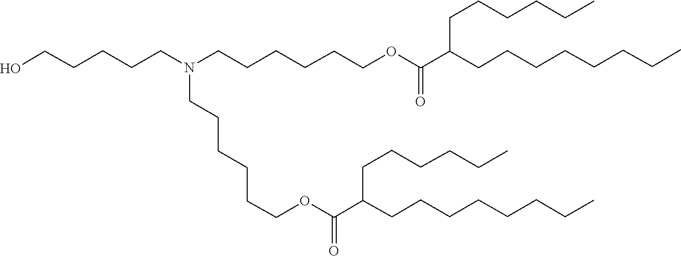 [SMILES: CCCCCCCCC(CCCCCC)C(=O)OCCCCCCN(CCCCCO)CCCCCCOC(=O)C(CCCCCC)CCCCCCCC] |
| III-8 | 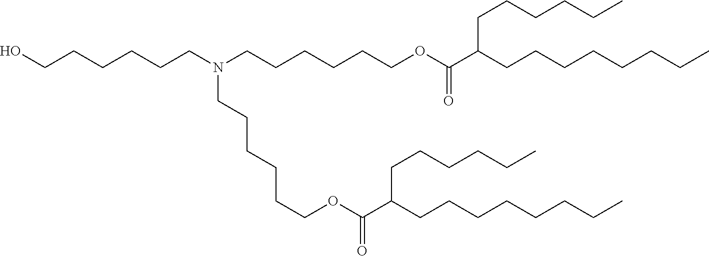 [SMILES: CCCCCCCCC(CCCCCC)C(=O)OCCCCCCN(CCCCCCO)CCCCCCOC(=O)C(CCCCCC)CCCCCCCC] |
| III-9 | 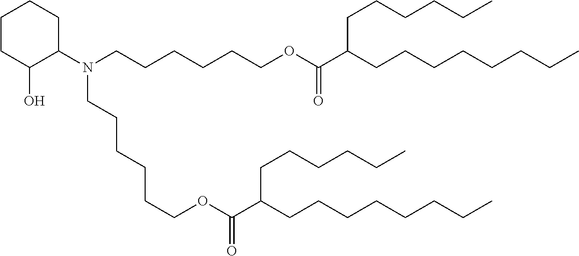 [SMILES: CCCCCCCCC(CCCCCC)C(=O)OCCCCCCN(CCCCCCOC(=O)C(CCCCCC)CCCCCCCC)C1CCCCC1O] |
| III-10 | 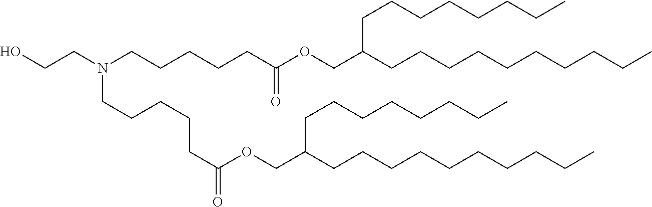 [SMILES: CCCCCCCCCCC(CCCCCCCC)COC(=O)CCCCCN(CCO)CCCCCC(=O)OCC(CCCCCCCC)CCCCCCCCCC] |
| III-11 | 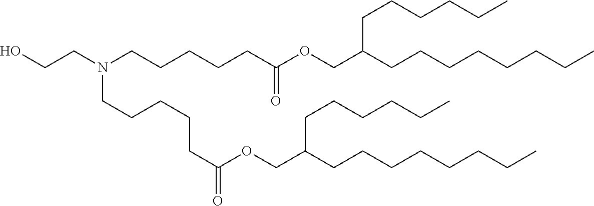 [SMILES: CCCCCCCCC(CCCCCC)COC(=O)CCCCCN(CCO)CCCCCC(=O)OCC(CCCCCC)CCCCCCCC] |
| III-12 | 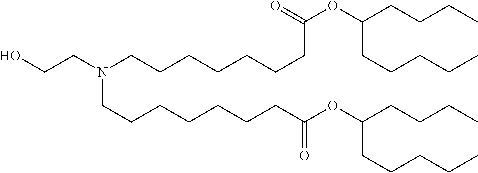 [SMILES: CCCCCC(CCCC)OC(=O)CCCCCCCN(CCO)CCCCCCCC(=O)OC(CCCC)CCCCC] |
| III-13 | 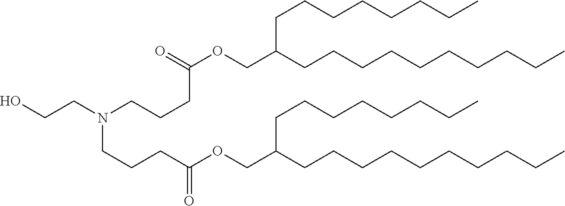 [SMILES: CCCCCCCCCCC(CCCCCCCC)COC(=O)CCCN(CCO)CCCC(=O)OCC(CCCCCCCC)CCCCCCCCCC] |
| III-14 | 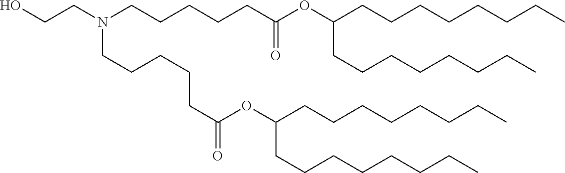 [SMILES: CCCCCCCCC(CCCCCCCC)OC(=O)CCCCCN(CCO)CCCCCC(=O)OC(CCCCCCCC)CCCCCCCC] |
| III-15 | 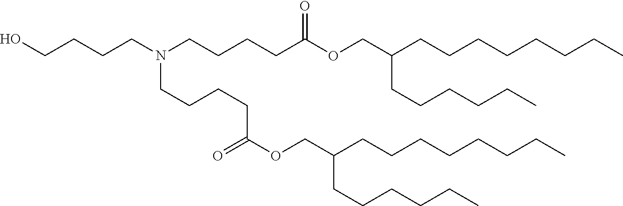 [SMILES: CCCCCCCCC(CCCCCC)COC(=O)CCCCN(CCCCO)CCCCC(=O)OCC(CCCCCC)CCCCCCCC] |
| III-16 | 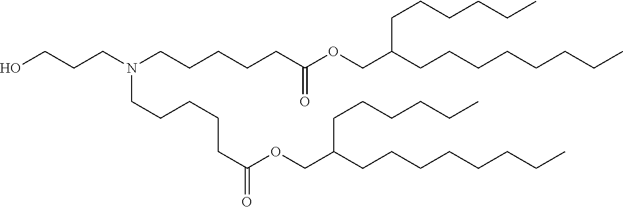 [SMILES: CCCCCCCCC(CCCCCC)COC(=O)CCCCCN(CCCO)CCCCCC(=O)OCC(CCCCCC)CCCCCCCC] |
| III-17 | 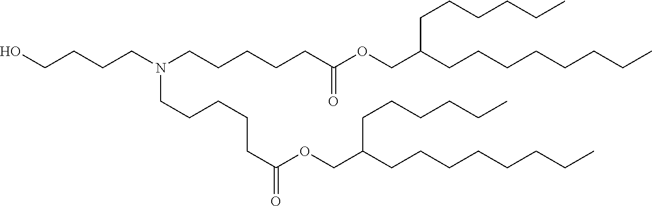 [SMILES: CCCCCCCCC(CCCCCC)COC(=O)CCCCCN(CCCCO)CCCCCC(=O)OCC(CCCCCC)CCCCCCCC] |
| III-18 | 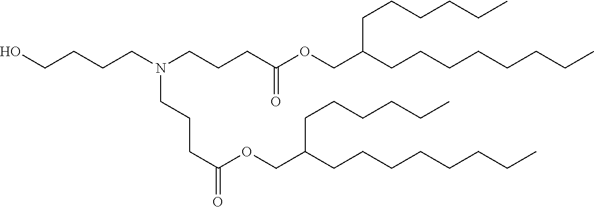 [SMILES: CCCCCCCCC(CCCCCC)COC(=O)CCCN(CCCCO)CCCC(=O)OCC(CCCCCC)CCCCCCCC] |
| III-19 | 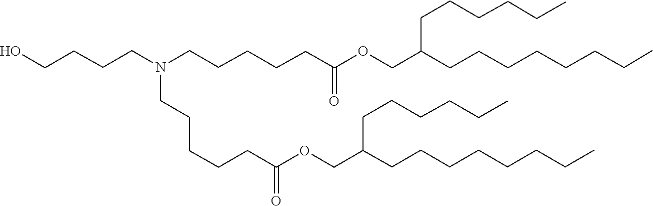 [SMILES: CCCCCCCCC(CCCCCC)COC(=O)CCCCCN(CCCCO)CCCCCC(=O)OCC(CCCCCC)CCCCCCCC] |
| III-20 | 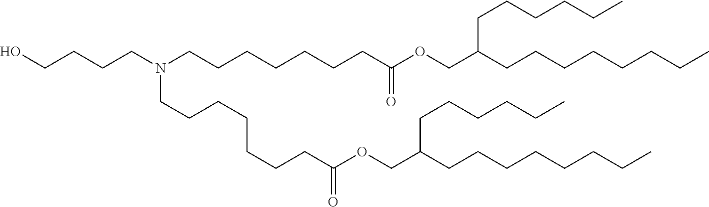 [SMILES: CCCCCCCCC(CCCCCC)COC(=O)CCCCCCCN(CCCCO)CCCCCCCC(=O)OCC(CCCCCC)CCCCCCCC] |
| III-21 | 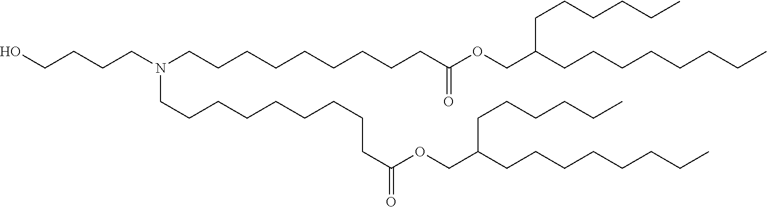 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 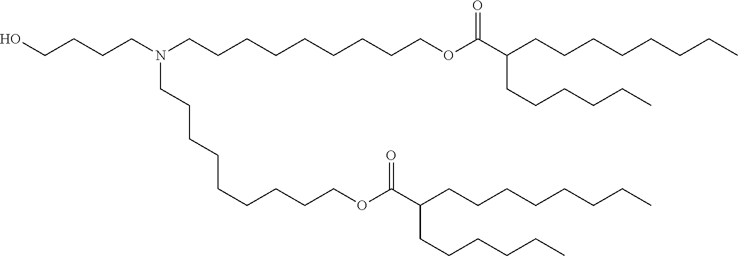 [SMILES: CCCCCCCCC(CCCCCC)C(=O)OCCCCCCCCCN(CCCCO)CCCCCCCCCOC(=O)C(CCCCCC)CCCCCCCC] |
| III-23 | 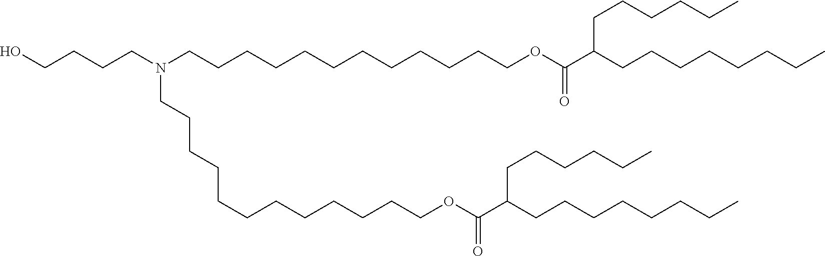 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 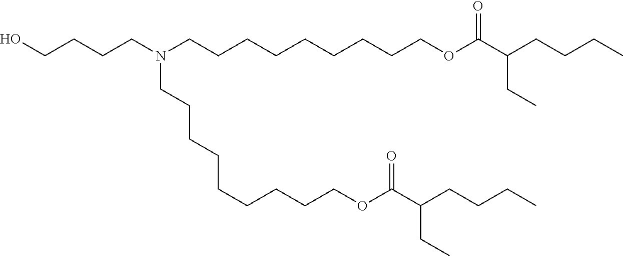 [SMILES: CCCCC(CC)C(=O)OCCCCCCCCCN(CCCCO)CCCCCCCCCOC(=O)C(CC)CCCC] |
| III-25 | 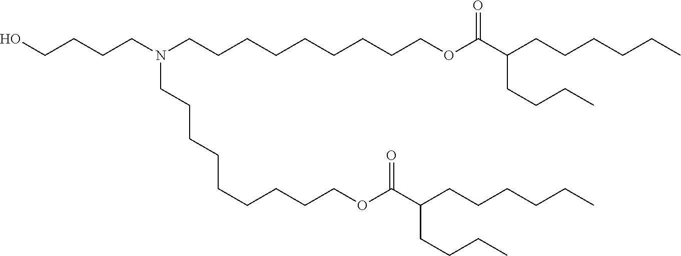 [SMILES: CCCCCCC(CCCC)C(=O)OCCCCCCCCCN(CCCCO)CCCCCCCCCOC(=O)C(CCCC)CCCCCC] |
| III-26 | 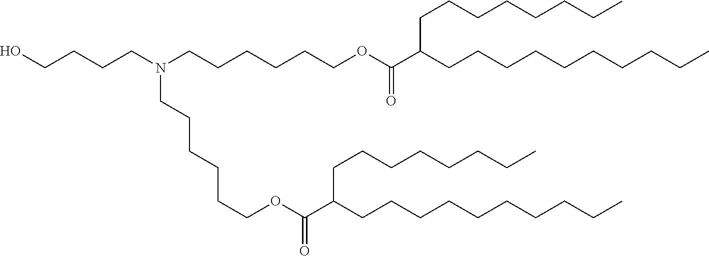 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 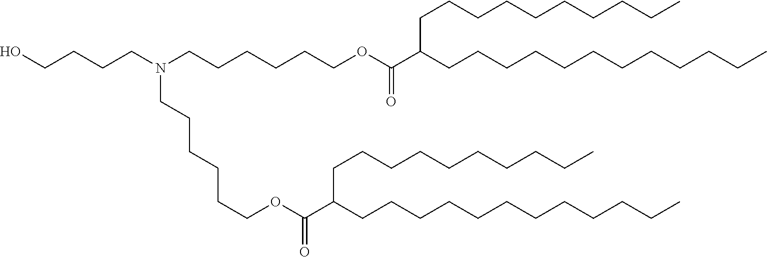 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 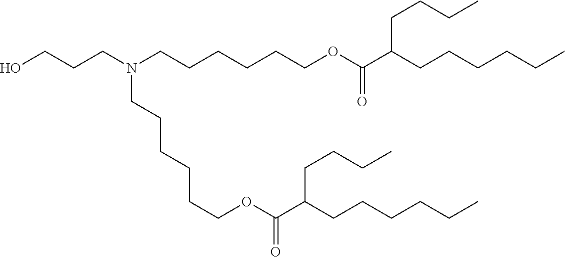 [SMILES: CCCCCCC(CCCC)C(=O)OCCCCCCN(CCCO)CCCCCCOC(=O)C(CCCC)CCCCCC] |
| III-29 | 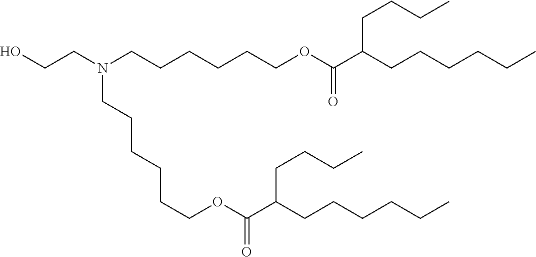 [SMILES: CCCCCCC(CCCC)C(=O)OCCCCCCN(CCO)CCCCCCOC(=O)C(CCCC)CCCCCC] |
| III-30 | 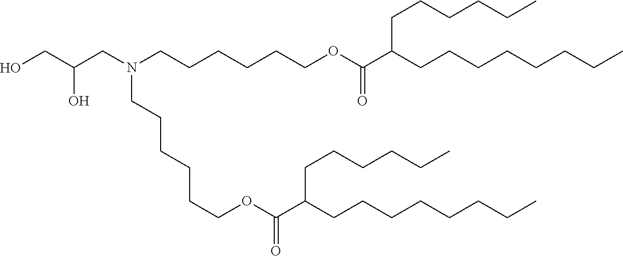 [SMILES: CCCCCCCCC(CCCCCC)C(=O)OCCCCCCN(CCCCCCOC(=O)C(CCCCCC)CCCCCCCC)CC(O)CO] |
| III-31 | 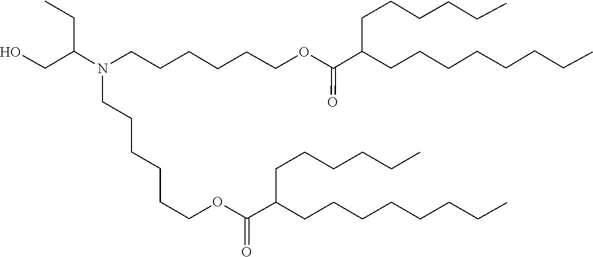 [SMILES: CCCCCCCCC(CCCCCC)C(=O)OCCCCCCN(CCCCCCOC(=O)C(CCCCCC)CCCCCCCC)C(CC)CO] |
| III-32 | 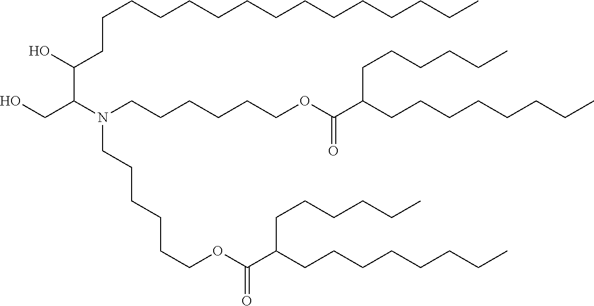 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 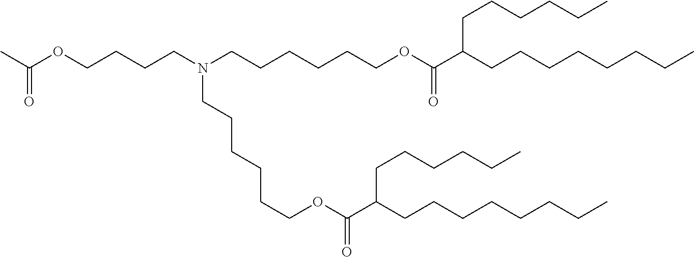 [SMILES: CCCCCCCCC(CCCCCC)C(=O)OCCCCCCN(CCCCCCOC(=O)C(CCCCCC)CCCCCCCC)CCCCOC(C)=O] |
| III-34 | 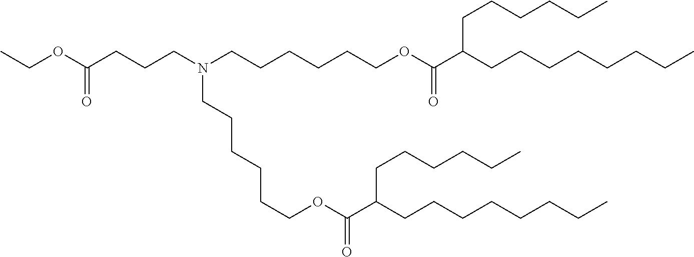 [SMILES: CCCCCCCCC(CCCCCC)C(=O)OCCCCCCN(CCCCCCOC(=O)C(CCCCCC)CCCCCCCC)CCCC(=O)OCC] |
| III-35 | 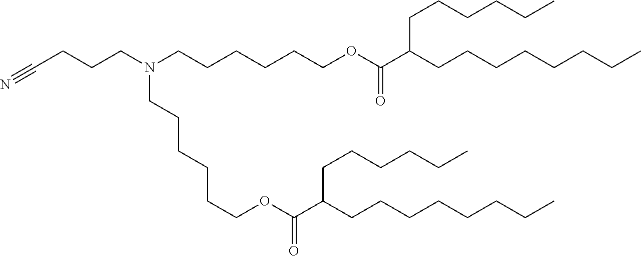 [SMILES: CCCCCCCCC(CCCCCC)C(=O)OCCCCCCN(CCCC#N)CCCCCCOC(=O)C(CCCCCC)CCCCCCCC] |
| III-36 | 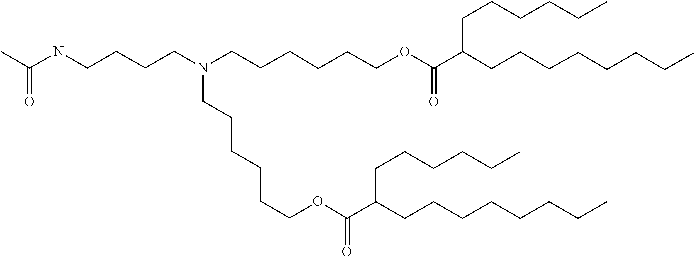 |

In various different embodiments, a cationic lipid has one of the structures set forth in Table 14 below.

TABLE 15
Exemplary Cationic Lipid Structures

| No. | Structure |
|---|---|
| A | 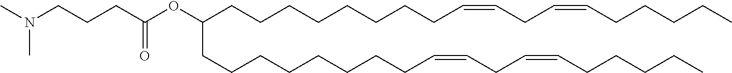 [SMILES: CCCCC/C=C\C/C=C\CCCCCCCCC(CCCCCCCC/C=C\C/C=C\CCCCC)OC(=O)CCCN(C)C] |
| B | 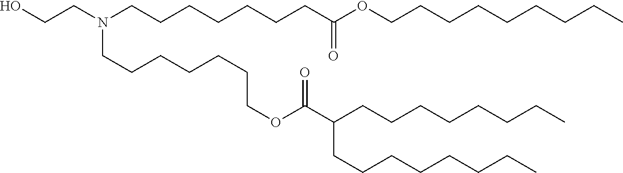 [SMILES: CCCCCCCCCOC(=O)CCCCCCCN(CCO)CCCCCCCOC(=O)C(CCCCCCCC)CCCCCCCC] |
| C | 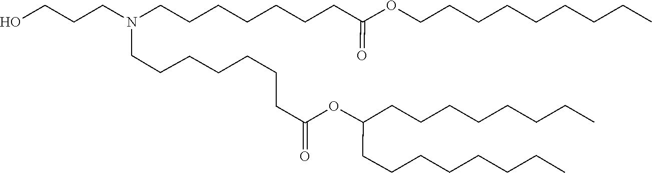 [SMILES: CCCCCCCCCOC(=O)CCCCCCCN(CCCO)CCCCCCCC(=O)OC(CCCCCCCC)CCCCCCCC] |
| D | 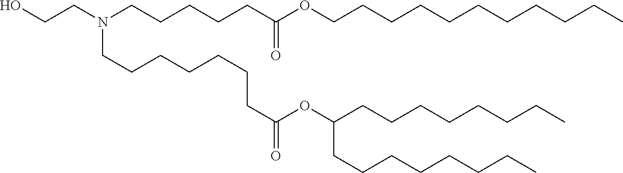 [SMILES: CCCCCCCCCCCOC(=O)CCCCCN(CCO)CCCCCCCC(=O)OC(CCCCCCCC)CCCCCCCC] |
| E | 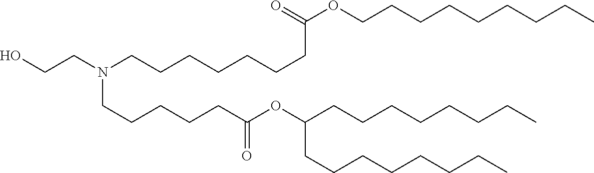 [SMILES: CCCCCCCCCOC(=O)CCCCCCCN(CCO)CCCCCC(=O)OC(CCCCCCCC)CCCCCCCC] |
| F | 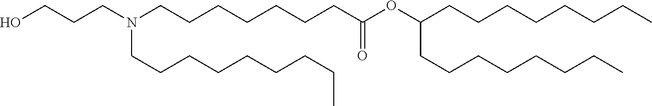 [SMILES: CCCCCCCCCN(CCCO)CCCCCCCC(=O)OC(CCCCCCCC)CCCCCCCC] |

In some embodiments, an LNP comprises a cationic lipid that is an ionizable lipid-like material (lipidoid). In some embodiments, a cationic lipid has the following structure:

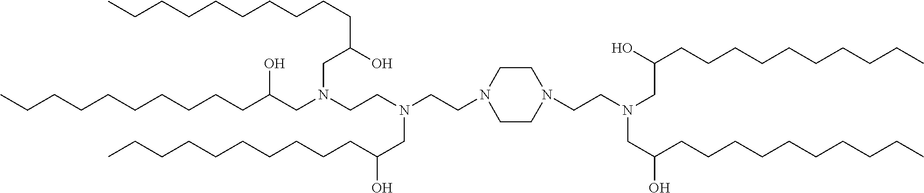 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments, lipid nanoparticles can have an average size (e.g., mean diameter) of about 30 nm to about 150 nm, about 40 nm to about 150 nm, about 50 nm to about 150 nm, about 60 nm to about 130 nm, about 70 nm to about 110 nm, about 70 nm to about 100 nm, about 70 to about 90 nm, or about 70 nm to about 80 nm. In some embodiments, lipid nanoparticles in accordance with the present disclosure can have an average size (e.g., mean diameter) of about 50 nm to about 100 nm. In some embodiments, lipid nanoparticles may have an average size (e.g., mean diameter) of about 50 nm to about 150 nm. In some embodiments, lipid nanoparticles may have an average size (e.g., mean diameter) of about 60 nm to about 120 nm. In some embodiments, lipid nanoparticles in accordance with the present disclosure can have an average size (e.g., mean diameter) of about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. The term “average diameter” or “mean diameter” refers to the mean hydrodynamic diameter of particles as measured by dynamic laser light scattering (DLS) with data analysis using the so-called cumulant algorithm, which provides as results the so-called Z-average with the dimension of a length, and the polydispersity index (PI), which is dimensionless (Koppel, D., J. Chem. Phys. 57, 1972, pp 4814-4820, ISO 13321, which is herein incorporated by reference). Here “average diameter,” “mean diameter,” “diameter,” or “size” for particles is used synonymously with this value of the Z-average.

In some embodiments, lipid nanoparticles described herein may exhibit a polydispersity index less than about 0.5, less than about 0.4, less than about 0.3, or about 0.2 or less. By way of example, lipid nanoparticles can exhibit a polydispersity index in a range of about 0.1 to about 0.3 or about 0.2 to about 0.3. The “polydispersity index” is preferably calculated based on dynamic light scattering measurements by the so-called cumulant analysis as mentioned in the definition of the “average diameter.” Under certain prerequisites, it can be taken as a measure of the size distribution of an ensemble of ribonucleic acid nanoparticles (e.g., ribonucleic acid nanoparticles).

Lipid nanoparticles described herein can be characterized by an “N/P ratio,” which is the molar ratio of cationic (nitrogen) groups (the “N” in N/P) in the cationic polymer to the anionic (phosphate) groups (the “P” in N/P) in RNA. It is understood that a cationic group is one that is either in cationic form (e.g., N⁺), or one that is ionizable to become cationic. Use of a single number in an N/P ratio (e.g., an N/P ratio of about 5) is intended to refer to that number over 1, e.g., an N/P ratio of about 5 is intended to mean 5:1. In some embodiments, a lipid nanoparticle described herein has an N/P ratio greater than or equal to 5. In some embodiments, a lipid nanoparticle described herein has an N/P ratio that is about 5, 6, 7, 8, 9, or 10. In some embodiments, an N/P ratio for a lipid nanoparticle described herein is from about 10 to about 50. In some embodiments, an N/P ratio for a lipid nanoparticle described herein is from about 10 to about 70. In some embodiments, an N/P ratio for a lipid nanoparticle described herein is from about 10 to about 120.

### B. Exemplary Methods of Making Lipid Nanoparticles

Lipids and lipid nanoparticles comprising nucleic acids and their method of preparation are known in the art, including, e.g., as described in U.S. Pat. Nos. 8,569,256, 5,965,542 and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2018/081480, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548, the full disclosures each of which are herein incorporated by reference in their entirety for the purposes described herein.

For example, in some embodiments, cationic lipids, neutral lipids (e.g., DSPC, and/or cholesterol) and polymer-conjugated lipids can be solubilized in ethanol at a pre-determined molar ratio (e.g., ones described herein). In some embodiments, lipid nanoparticles (lipid nanoparticle) are prepared at a total lipid to polyribonucleotides weight ratio of approximately 10:1 to 30:1. In some embodiments, such polyribonucleotides can be diluted to 0.2 mg/mL in acetate buffer.

In some embodiments, using an ethanol injection technique, a colloidal lipid dispersion comprising polyribonucleotides can be formed as follows: an ethanol solution comprising lipids, such as cationic lipids, neutral lipids, and polymer-conjugated lipids, is injected into an aqueous solution comprising polyribonucleotides (e.g., ones described herein).

In some embodiments, lipid and polyribonucleotide solutions can be mixed at room temperature by pumping each solution at controlled flow rates into a mixing unit, for example, using piston pumps. In some embodiments, the flow rates of a lipid solution and a RNA solution into a mixing unit are maintained at a ratio of 1:3. Upon mixing, nucleic acid-lipid particles are formed as the ethanolic lipid solution is diluted with aqueous polyribonucleotides. The lipid solubility is decreased, while cationic lipids bearing a positive charge interact with the negatively charged RNA.

In some embodiments, a solution comprising RNA-encapsulated lipid nanoparticles can be processed by one or more of concentration adjustment, buffer exchange, formulation, and/or filtration.

In some embodiments, RNA-encapsulated lipid nanoparticles can be processed through filtration.

In some embodiments, particle size and/or internal structure of lipid nanoparticles (with or without RNAs) may be monitored by appropriate techniques such as, e.g., small-angle X-ray scattering (SAXS) and/or transmission electron cryomicroscopy (CryoTEM).

### V. Pharmaceutical Compositions

The present disclosure provides compositions e.g., pharmaceutical compositions comprising one or more polyribonucleotides as described herein.

In some embodiments, pharmaceutical formulations comprise an active agent and one or more excipients or carriers.

In some embodiments, an active agent may be or comprise an HSV (e.g., HSV-1 and/or HSV-2) antigen—e.g., that is or comprises an HSV (e.g., HSV-1 and/or HSV-2) protein or antigen or an antigenic fragment or epitope thereof, as described herein. Thus, in some embodiments, an active agent is a polypeptide or plurality of polypeptides. In some embodiments, a polypeptide active agent includes a plurality of HSV (e.g., HSV-1 and/or HSV-2) antigens (e.g., from a single HSV (e.g., HSV-1 and/or HSV-2 protein or from a plurality of different HSV (e.g., HSV-1 and/or HSV-2) proteins). In some embodiments, a polypeptide active agent is or comprises at least one peptide that represents a distinct HSV (e.g., HSV-1 and/or HSV-2) antigen. In some embodiments, a polypeptide active agent includes at least one peptide that is or comprises an antigen or antigenic fragment or epitope of an HSV (e.g., HSV-1 and/or HSV-2) protein; in some such embodiments, the polypeptide active agent does not include any full-length HSV (e.g., HSV-1 and/or HSV-2) protein.

In some embodiments, an active agent may be or comprise a cell population—for example a population of cells that expresses (e.g., internally, on its surface, and or secreting) at least one antigen as described herein. Alternatively or additionally, a population of cells (e.g., antigen presenting cells such as dendritic cells) that are loaded with (e.g., bound in MHC complexes) HSV (e.g., HSV-1 and/or HSV-2) antigen peptides as described herein.

In some embodiments, an active agent is a polynucleotide that encodes (or is complementary to one that encodes) an HSV (e.g., HSV-1 and/or HSV-2) antigen as described herein. In some such embodiments, a polynucleotide is single-stranded; in other embodiments, a polynucleotide is double stranded. In some embodiments, a polynucleotide active agent is DNA (e.g., a DNA viral vector, such as an adenoviral, adeno-associated viral, baculoviral, poxviral [e.g., vaccinia viral] vector); in some embodiments, a polynucleotide active agent is RNA (e.g., a lentiviral vector or, more preferably, an mRNA construct as described herein).

In many embodiments, a polynucleotide active agent is RNA and is provided and/or utilized in a lipid composition such as a lipoplex preparation or, preferably, an LNP preparation.

In some embodiments, a provided formulation is a liquid formulation. In some embodiments, a provided formulation is a solid (e.g., frozen formulation. In some embodiments, a provided formulation is a dry formulation.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, an excipient is approved for use in humans and for veterinary use. In some embodiments, an excipient is approved by the United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

General considerations in the formulation and/or manufacture of pharmaceutical agents may be found, for example, in Remington: The Science and Practice of Pharmacy 21st ed., Lippincott Williams & Wilkins, 2005 (incorporated herein by reference).

In some embodiments, pharmaceutical compositions provided herein may be formulated with one or more pharmaceutically acceptable carriers or diluents as well as any other known adjuvants and excipients in accordance with conventional techniques such as those disclosed in Remington: The Science and Practice of Pharmacy 21st ed., Lippincott Williams & Wilkins, 2005 (incorporated herein by reference).

Pharmaceutical compositions described herein can be administered by appropriate methods known in the art. As will be appreciated by a skilled artisan, the route and/or mode of administration may depend on a number of factors, including, e.g., but not limited to stability and/or pharmacokinetics and/or pharmacodynamics of pharmaceutical compositions described herein.

In some embodiments, pharmaceutical compositions described herein are formulated for parenteral administration, which includes modes of administration other than enteral and topical administration, usually by injection, and includes, without limitation, intravenous, intramuscular, intraarterial, intradermal, subcutaneous, subcuticular, or intraarticular injection and infusion. In preferred embodiments, pharmaceutical compositions described herein are formulated for intravenous, intramuscular, or subcutaneous administration. In particularly preferred embodiments, pharmaceutical compositions described herein are formulated for intramuscular administration.

In some embodiments, pharmaceutical compositions described herein are formulated for intravenous administration. In some embodiments, pharmaceutically acceptable excipients that may be useful for intravenous administration include sterile aqueous solutions or dispersions and sterile powders for preparation of sterile injectable solutions or dispersions.

Therapeutic compositions typically must be sterile and stable under the conditions of manufacture and storage. The composition can be formulated as a solution, microemulsion, lipid nanoparticles, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. Proper fluidity can be maintained, for example, by the use of surfactants. In many cases, it will be preferable to include isotonic agents, for example, sugars, polyalcohols such as mannitol, sorbitol, or sodium chloride in the composition. In some embodiments, prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization and/or microfiltration. In some embodiments, pharmaceutical compositions can be prepared as described herein and/or methods known in the art. In some embodiments, a pharmaceutical composition includes ALC-0315; ALC-0159; DSPC; Cholesterol; Sucrose; NaCl; KCl; Na₂HPO₄; KH₂PO₄; Water for injection. In some embodiments, normal saline (isotonic 0.9% NaCl) is used as diluent.

These compositions may also contain adjuvants such as preservatives, wetting agents, emulsifying agents and dispersing agents. Prevention of the presence of microorganisms may be ensured both by sterilization procedures, and by the inclusion of various antibacterial and antifungal agents, for example, paraben, chlorobutanol, phenol sorbic acid, and the like. It may also be desirable to include isotonic agents, such as sugars, sodium chloride, and the like into pharmaceutical compositions described herein. In addition, prolonged absorption of the injectable pharmaceutical form may be brought about by the inclusion of agents which delay absorption such as aluminum monostearate and gelatin.

Formulations of pharmaceutical compositions described herein may be prepared by any method known or hereafter developed in the art of pharmacology. In general, such preparatory methods include the step of bringing active ingredient(s) into association with a diluent or another excipient and/or one or more other accessory ingredients, and then, if necessary and/or desirable, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of at least one RNA product produced using a system and/or method described herein.

Relative amounts of polyribonucleotides encapsulated in lipid nanoparticles, a pharmaceutically acceptable excipient, and/or any additional ingredients in a pharmaceutical composition can vary, depending upon the subject to be treated, target cells, diseases or disorders, and may also further depend upon the route by which the composition is to be administered.

In some embodiments, pharmaceutical compositions described herein are formulated into pharmaceutically acceptable dosage forms by conventional methods known to those of skill in the art. Actual dosage levels of the active ingredients (e.g., polyribonucleotides encapsulated in lipid nanoparticles) in the pharmaceutical compositions described herein may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A physician having ordinary skill in the art can readily determine and prescribe the effective amount of the pharmaceutical composition required. For example, a physician could start doses of active ingredients (e.g., polyribonucleotides encapsulated in lipid nanoparticles) employed in the pharmaceutical composition at levels lower than that required in order to achieve the desired therapeutic effect and gradually increase the dosage until the desired effect is achieved.

In some embodiments, a pharmaceutical composition is formulated (e.g., but not limited to, for intravenous, intramuscular, or subcutaneous administration) to deliver a dose of about 5 mg RNA/kg.

In some embodiments, a pharmaceutical composition described herein may further comprise one or more additives, for example, in some embodiments that may enhance stability of such a composition under certain conditions. Examples of additives may include but are not limited to salts, buffer substances, preservatives, and carriers. For example, in some embodiments, a pharmaceutical composition may further comprise a cryoprotectant (e.g., sucrose) and/or an aqueous buffered solution, which may in some embodiments include one or more salts, including, e.g., alkali metal salts or alkaline earth metal salts such as, e.g., sodium salts, potassium salts, and/or calcium salts.

In some embodiments, a pharmaceutical composition provided herein is a preservative-free, sterile RNA-lipid nanoparticle dispersion in an aqueous buffer for intravenous or intramuscular administration.

Although the descriptions of pharmaceutical compositions provided herein are principally directed to pharmaceutical compositions that are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation.

### VI. Characterization

Without wishing to be bound by any particular theory, it is proposed that ability to induce CD8⁺ T cells may be important to effectiveness of a composition for the treatment of an HSV (e.g., HSV-1 and/or HSV-2) infection, (e.g., a pharmaceutical composition, an immunogenic composition, or a vaccine). Alternatively or additionally, in some embodiments, a robust antibody response may be required for effectiveness. In some embodiments, it may be that both are required or useful.

In some embodiments, provided technologies (e.g., compositions and/or dosing regimens, etc) are characterized by an ability to induce (e.g., when administered to a model system and/or to a human, for example by parenteral administration such as by intramuscular administration) an immune response characterized by CD8⁺ T cells targeting one or more HSV (e.g., HSV-1 and/or HSV-2) antigen(s) described herein. That is, in some embodiments, provided technologies are characterized in that, when administered (e.g., by parenteral administration such as by intramuscular administration) to an organism (e.g., a model organism or an animal or human organism in need of protection), provided technologies induce CD8+ T cells targeting one or more HSV (e.g., HSV-1 and/or HSV-2) antigens. In some embodiments, provided technologies are characterized in that they induce a greater CD8⁺ T cell response with respect to one or more HSV (e.g., HSV-1 and/or HSV-2) antigens and/or induce a CD8+ T cell response with broader diversity (e.g., with detectable and/or significant binding to a larger number of different T cell antigens) than is observed for vaccines, e.g., in Table 1 or another appropriate reference.

In some embodiments, provided technologies are characterized in that they induce gammadelta T cells. As those skilled in the art are aware, gammadelta T cells typically represent only a small fraction (e.g., up to about 5%) of an overall T cell population in an organism. Gammadelta T cells express TCR chains encoded by the gamma and delta gene loci; subsets of gamma delta T cells are defined by the inclusion of invariant TCR V-(D)-J segments and are tissue- or context-specific. Gammadelta T cells secrete particular effector cytokines in a subtype- and context-specific manner. Often, gammadelta T cells express certain markers (e.g., as Fc gamma RIII/CD16 and Toll-like receptors that are often associated with natural killer cells and/or antigen-presenting cells. Gammadelta T cells typically lack CD4 and CD8.

In some embodiments, provided technologies are characterized in that they induce polyclonal high affinity antibodies.

In some embodiments, provided technologies are characterized in that they induce antibody titers to a level that provides sufficient protective response against HSV, when administered to a relevant population.

In some embodiments, provided technologies are characterized in that they induce sterile protection, e.g., when evaluated in a model system such as a mouse modes.

In some embodiments, provided technologies are characterized in that they induce a CD4+T helper cell response and/or CD8+ T cell memory responses (e.g., promoting development and/or expansion of memory CD8+ T cells.

In some embodiments, provided compositions are assessed as described herein, for example for RNA integrity, stability, level, capping efficiency, translatability, of RNA, etc and/or for one or more properties of a composition (e.g., an LNP preparation) such as, for example ability to induce an antibody response, a T cell response, a T cell response with particular features (e.g., level of antibody to one or more antigens, persistence of such level, diversity of elicited antibodies, type and/or diversity of T cell response, etc.

In some embodiments, provided formulations are identified and/or characterized with respect to one or more activities or features, including, for example, expression level, nature of immune response, level of protection (e.g., to challenge, impact on viral load, impact on health and/or survival), immunogenicity (e.g., assessment of cytokine responses, phenotyping of immune response, T cell depletion and/or protection), serology, and/or functional antibody responses. In some embodiments, assessment of provided compositions can be performed in an animal model. In many embodiments, assessment of provided compositions in human system(s) is desirable. In some embodiments, in vitro assessments are performed in human systems. To give but a few examples, in some embodiments, presentation of provided antigen(s) or antigenic fragments or epitopes thereof by human dendritic cells to stimulate human T cells is assessed in vitro. Alternatively or additionally, in some embodiments, in vitro binding of sera from infected humans to provided antigens is assessed.

In some embodiments, in vivo assessments are performed in human systems. For example, in some embodiments, one or more human trials are performed. In such trials, healthy humans (e.g., volunteers, who have typically undergone screening and/or consenting procedures) are treated with a provided composition (e.g., an immunostimulatory composition, e.g., a vaccine composition) and subsequently are inoculated with HSV (e.g., HSV-1 and/or HSV-2), and clinically monitored to assess level of protection, e.g., from established infection and/or symptomatic or serious disease. Alternatively or additionally, in some embodiments, subjects are monitored for responsiveness (e.g., increased responsiveness) to a particular known or potential anti-HSV (e.g., anti-HSV-I and/or anti-HSV-2) therapy.

In some embodiments, a provided composition (e.g., an immunostimulatory composition, e.g., a vaccine composition) provides significant (e.g., at least about 60%, 65%, 70%, 75%, 80%, 85%, 90% or more protection from one or more of established infection, symptomatic disease, and/or serious disease. In some embodiments, a provided composition (e.g., an immunostimulatory composition, e.g., a vaccine composition) provides significantly increased responsiveness to therapy, for example as may be assessed by one or more of delayed onset, reduction of severity, and/or faster resolution of one or more symptoms or characteristics of infection.

### VII. Patient Populations

In some aspects, technologies of the present disclosure are used for therapeutic and/or prophylactic purposes. In some embodiments, technologies of the present disclosure are used in the treatment and/or prophylactic of an HSV infection (e.g., HSV-1 and/or HSV-2 infection). Prophylactic purposes of the present disclosure comprises pre-exposure prophylaxis and/or post-exposure prophylaxis.

In some embodiments, technologies of the present disclosure are used in the treatment and/or prophylaxis of a disorder related to such an HSV (e.g., HSV-1 and/or HSV-2) infection. A disordered related to such an HSV (e.g., HSV-1 and/or HSV-2) infection comprises, for example, a typical symptom and/or a complication of an HSV (e.g., HSV-1 and/or HSV-2) infection.

In some embodiments, provided compositions (e.g., that are or comprise HSV (e.g., HSV-1 and/or HSV-2) antigens) may be useful to detect and/or characterize one or more features of an anti-HSV (e.g., anti-HSV-1 and/or anti-HSV-2) immune response (e.g., by detecting binding to a provided antigen by serum from an infected subject).

In some embodiments, provided compositions (e.g., that are or comprise HSV (e.g., HSV-1 and/or HSV-2) antigens) are useful to raise antibodies to one or more antigens included therein; such antibodies may themselves be useful, for example for detection or treatment of an HSV (e.g., HSV-1 and/or HSV-2) or infection thereby.

The present disclosure provides use of encoding nucleic acids (e.g., DNA or RNA) to produce encoded antigens and/or use of DNA constructs to produce RNA.

In some embodiments, technologies of the present disclosure are utilized in a non-limited subject population; in some embodiments, technologies of the present disclosure are utilized in particular subject populations.

In some embodiments, a subject population comprises an adult population. In some embodiments, an adult population comprises subjects between the ages of about 19 years and about 60 years of age (e.g., about 20, 25, 30, 35, 40, 45, 50, 55, or 60 years of age).

In some embodiments, a subject population comprises an elderly population. In some embodiments, an elderly population comprises subjects of about 60 years of age, about 70 years of age, or older (e.g., about 65, 70, 75, 80, 85, 90, 95, or 100 years of age).

In some embodiments, a subject population comprises a pediatric population. In some embodiments, a pediatric population comprises subjects approximately 18 years old or younger. In some such embodiments, a pediatric population comprises subjects between the ages of about 1 year and about 18 years (e.g., 1, 2, 3, 4, 5, 6, 7, 8, 9, 10, 11, 12, 13, 14, 15, 16, 17, or 18 years of age).

In some embodiments, a subject population comprises a newborn population. In some embodiments, a newborn population comprises subjects about 12 months or younger (e.g., 12, 11, 10, 9, 8, 7, 6, 5, 4, 3, 2, 1 months or younger). In some embodiments, subject populations to be treated with technologies described herein include infants (e.g., about 12 months or younger) whose mothers did not receive such technologies described herein during pregnancy. In some embodiments, subject populations to be treated with technologies described herein may include pregnant women; in some embodiments, infants whose mothers were treated with disclosed technologies during pregnancy (e.g., who received at least one dose, or alternatively only who received both doses), are not vaccinated during the first weeks, months, or even years (e.g., 1, 2, 3, 4, 5, 6, 7, 8 weeks or more, or 1, 2, 3, 4, 5, 6, 7, 8, 9, 10, 11, 12, 13, 14, 15, 16, 17, 18, 19, 20, 21, 22, 23, 24 months or more, or 1, 2, 3, 4, 5 years or more) post-birth. Alternatively or additionally, in some embodiments, infants whose mothers were treated with disclosed technologies during pregnancy (e.g., who received at least one dose, or alternatively only who received both doses), receive reduced treated with disclosed technologies (e.g., lower doses and/or smaller numbers of administrations—e.g., boosters—and/or lower total exposure over a given period of time) after birth, for example during the first weeks, months, or even years (e.g., 1, 2, 3, 4, 5, 6, 7, 8 weeks or more, or 1, 2, 3, 4, 5, 6, 7, 8, 9, 10, 11, 12, 13, 14, 15, 16, 17, 18, 19, 20, 21, 22, 23, 24 months or more, or 1, 2, 3, 4, 5 years or more) post-birth or may need reduced vaccination (e.g., lower doses and/or smaller numbers of administrations—e.g., boosters—over a given period of time), In some embodiments, compositions as provided herein are administered to subject populations that do not include pregnant women.

In some embodiments, a subject population is or comprises children aged 6 weeks to up to 17 months of age.

In some embodiments, a provided pharmaceutical composition (e.g., immunogenic composition, e.g., vaccine) may be administered in combination with (i.e., so that subject(s) are simultaneously exposed to both) another pharmaceutical composition (e.g., immunogenic composition, e.g., vaccine) or therapeutic intervention, e.g., to treat or prevent HSV (e.g., HSV-1 and/or HSV-2) infection, or another disease, disorder, or condition.

In some embodiments, a provided pharmaceutical composition (e.g., immunogenic composition, e.g., vaccine) may be administered with a protein vaccine, a DNA vaccine, an RNA vaccine, a cellular vaccine, a conjugate vaccine, etc. In some embodiments, one or more doses of a provided pharmaceutical composition (e.g., immunogenic composition, e.g., vaccine) may be administered together with (e.g., in a single visit) another vaccine or other therapy.

In some embodiments, a provided pharmaceutical composition (e.g., immunogenic composition, e.g., vaccine) may be administered to subjects who have been exposed, or expect they have been exposed, to HSV (e.g., HSV-1 and/or HSV-2). In some embodiments, a provided pharmaceutical composition (e.g., immunogenic composition, e.g., vaccine) may be administered to subjects who do not have symptoms of an HSV (e.g., HSV-1 and/or HSV-2) infection.

### VIII. Treatment Methods

In some embodiments, technologies of the present disclosure may be administered to subjects according to a particular dosing regimen. In some embodiments, a dosing regimen may involve a single administration; in some embodiments, a dosing regimen may comprise one or more “booster” administrations after the initial administration. In some embodiments, initial and boost doses are the same amount; in some embodiments they differ. In some embodiments, two or more booster doses are administered. In some embodiments, a plurality of doses are administered at regular intervals. In some embodiments, periods of time between doses become longer. In some embodiments, one or more subsequent doses is administered if a particular clinical (e.g., reduction in neutralizing antibody levels) or situational (e.g., local development of a new strain) even arises or is detected.

In some embodiments, administered pharmaceutical compositions (e.g., immunogenic compositions, e.g., vaccines) comprising RNA constructs that encode HSV (e.g., HSV-1 and/or HSV-2) antigen(s) are administered in RNA doses of from about 0.1 μg to about 300 μg, about 0.5 μg to about 200 μg, or about 1 μg to about 100 μg, such as about 1 μg, about 3 μg, about 10 μg, about 30 μg, about 50 μg, or about 100 μg. In some embodiments, an saRNA construct is administered at a lower dose (e.g., 2, 4, 5, 10 fold or more lower) than a modRNA or uRNA construct.

In some embodiments, a first booster dose is administered within a about six months of the initial dose, and preferably within about 5, 4, 3, 2, or 1 months. In some embodiments, a first booster dose is administered in a time period that begins about 1, 2, 3, or 4 weeks after the first dose, and ends about 2, 3, 4, 5, 6, 7, 8, 9, 10, 11, 12 weeks of the first dose (e.g., between about 1 and about 12 weeks after the first dose, or between about 2 or 3 weeks and about 5 and 6 weeks after the first dose, or about 3 weeks or about 4 weeks after the first dose).

In some embodiments, a plurality of booster doses (e.g., 2, 3, or 4) doses are administered within 6 months of the first dose, or within 12 months of the first dose.

In some embodiments, 3 doses or fewer are required to achieve effective vaccination (e.g., greater than 60%, and in some embodiments greater than about 70%, about 75%, about 80%, about 85%, about 90% or more) reduction in risk of infection, or of serious disease. In some embodiments, not more than two doses are required. In some embodiments, a single dose is sufficient. In some embodiments, an RNA dose is about 60 μg or lower, 50 μg or lower, 40 μg or lower, 30 μg or lower, 20 μg or lower, 10 μg or lower, 5 μg or lower, 2.5 μg or lower, or 1 μg or lower. In some embodiments, an RNA dose is about 0.25 μg, at least 0.5 μg, at least 1 μg, at least 2 μg, at least 3 μg, at least 4 μg, at least 5 μg, at least 10 μg, at least 20 μg, at least 30 μg, or at least 40 μg. In some embodiments, an RNA dose is about 0.25 μg to 60 μg, 0.5 μg to 55 μg, 1 μg to 50 μg, 5 μg to 40 μg, or 10 μg to 30 μg may be administered per dose. In some embodiments, an RNA dose is about 30 μg. In some embodiments, at least two such doses are administered. For example, a second dose may be administered about 21 days following administration of the first dose. In some embodiments, a first booster dose is administered about one month after an initial dose. In some such embodiments, at least one further booster is administered at one-month interval(s). In some embodiments, after 2 or 3 boosters, a longer interval is introduced and no further booster is administered for at least 6, 9, 12, 18, 24, or more months. In some embodiments, a single further booster is administered after about 18 months. In some embodiments, no further booster is required unless, for example, a material change in clinical or environmental situation is observed.

### IX. Methods of Manufacture

Individual polyribonucleotides can be produced by methods known in the art. For example, in some embodiments, polyribonucleotides can be produced by in vitro transcription, for example, using a DNA template. A plasmid DNA used as a template for in vitro transcription to generate a polyribonucleotide described herein is also within the scope of the present disclosure.

A DNA template is used for in vitro RNA synthesis in the presence of an appropriate RNA polymerase (e.g., a recombinant RNA-polymerase such as a T7 RNA-polymerase) with ribonucleotide triphosphates (e.g., ATP, CTP, GTP, UTP). In some embodiments, polyribonucleotides (e.g., ones described herein) can be synthesized in the presence of modified ribonucleotide triphosphates. By way of example only, in some embodiments, pseudouridine (ψ), N1-methyl-pseudouridine (m1ψ), or 5-methyl-uridine (m5U) can be used to replace uridine triphosphate (UTP). In some embodiments, pseudouridine (ψ) can be used to replace uridine triphosphate (UTP). In some embodiments, N₁-methyl-pseudouridine (m1ψ) can be used to replace uridine triphosphate (UTP). In some embodiments, 5-methyl-uridine (m5U) can be used to replace uridine triphosphate (UTP).

As will be clear to those skilled in the art, during in vitro transcription, an RNA polymerase (e.g., as described and/or utilized herein) typically traverses at least a fragment of a single-stranded DNA template in the 3′→5′ direction to produce a single-stranded complementary RNA in the 5′→3′ direction.

In some embodiments where a polyribonucleotide comprises a polyA tail, one of those skill in the art will appreciate that such a polyA tail may be encoded in a DNA template, e.g., by using an appropriately tailed PCR primer, or it can be added to a polyribonucleotide after in vitro transcription, e.g., by enzymatic treatment (e.g., using a poly(A) polymerase such as an E. coli Poly(A) polymerase). Suitable poly(A) tails are described herein above. For example, in some embodiments, a poly(A) tail comprises a nucleotide sequence of AAAAAAAAAAAAAAAAAAAAAAAAAAAAAAGCATATGACTAAAAAAAAAAAA AAAAAAAAAAAAAAAAAAAAAAAAAAAAAAAAAAAAAAAAAAAAAAAAAAA AAAAAAA (SEQ ID NO: 210). In some embodiments, a poly(A) tail comprises a plurality of A residues interrupted by a linker. In some embodiments, a linker comprises the nucleotide sequence GCATATGAC (SEQ ID NO: 211).

In some embodiments, those skilled in the art will appreciate that addition of a 5′ cap to an RNA (e.g., mRNA) can facilitate recognition and attachment of the RNA to a ribosome to initiate translation and enhances translation efficiency. Those skilled in the art will also appreciate that a 5′ cap can also protect an RNA product from 5′ exonuclease mediated degradation and thus increases half-life. Methods for capping are known in the art; one of ordinary skill in the art will appreciate that in some embodiments, capping may be performed after in vitro transcription in the presence of a capping system (e.g., an enzyme-based capping system such as, e.g., capping enzymes of vaccinia virus). In some embodiments, a cap may be introduced during in vitro transcription, along with a plurality of ribonucleotide triphosphates such that a cap is incorporated into a polyribonucleotide during transcription (also known as co-transcriptional capping). In some embodiments, a GTP fed-batch procedure with multiple additions in the course of the reaction may be used to maintain a low concentration of GTP in order to effectively cap the RNA. Suitable 5′ cap are described herein above. For example, in some embodiments, a 5′ cap comprises m7(3′OMeG)(5′)ppp(5′)(2′OMeA)pG.

Following RNA transcription, a DNA template is digested. In some embodiments, digestion can be achieved with the use of DNase I under appropriate conditions.

In some embodiments, in-vitro transcribed polyribonucleotides may be provided in a buffered solution, for example, in a buffer such as HEPES, a phosphate buffer solution, a citrate buffer solution, an acetate buffer solution; in some embodiments, such solution may be buffered to a pH within a range of, for example, about 6.5 to about 7.5; in some embodiments approximately 7.0. In some embodiments, production of polyribonucleotides may further include one or more of the following steps: purification, mixing, filtration, and/or filling.

In some embodiments, polyribonucleotides can be purified (e.g., in some embodiments after in vitro transcription reaction), for example, to remove components utilized or formed in the course of the production, like, e.g., proteins, DNA fragments, and/or or nucleotides. Various nucleic acid purifications that are known in the art can be used in accordance with the present disclosure. Certain purification steps may be or include, for example, one or more of precipitation, column chromatography (including, e.g., but not limited to anionic, cationic, hydrophobic interaction chromatography (HIC)), solid substrate-based purification (e.g., magnetic bead-based purification). In some embodiments, polyribonucleotides may be purified using magnetic bead-based purification, which in some embodiments may be or comprise magnetic bead-based chromatography. In some embodiments, polyribonucleotides may be purified using hydrophobic interaction chromatography (HIC) and/or diafiltration. In some embodiments, polyribonucleotides may be purified using HIC followed by diafiltration.

In some embodiments, dsRNA may be obtained as side product during in vitro transcription. In some such embodiments, a second purification step may be performed to remove dsRNA contamination. For example, in some embodiments, cellulose materials (e.g., microcrystalline cellulose) may be used to remove dsRNA contamination, for examples in some embodiments in a chromatographic format. In some embodiments, cellulose materials (e.g., microcrystalline cellulose) can be pretreated to inactivate potential RNase contamination, for example in some embodiments by autoclaving followed by incubation with aqueous basic solution, e.g., NaOH. In some embodiments, cellulose materials may be used to purify polyribonucleotides according to methods described in WO 2017/182524, the entire content of which is incorporated herein by reference.

In some embodiments, a batch of polyribonucleotides may be further processed by one or more steps of filtration and/or concentration. For example, in some embodiments, polyribonucleotide(s), for example, after removal of dsRNA contamination, may be further subject to diafiltration (e.g., in some embodiments by tangential flow filtration), for example, to adjust the concentration of polyribonucleotides to a desirable RNA concentration and/or to exchange buffer to a drug substance buffer.

In some embodiments, polyribonucleotides may be processed through 0.2 μm filtration before they are filled into appropriate containers.

In some embodiments, polyribonucleotides and compositions thereof may be manufactured in accordance with a process as described herein, or as otherwise known in the art.

In some embodiments, polyribonucleotides and compositions thereof may be manufactured at a large scale. For example, in some embodiments, a batch of polyribonucleotides can be manufactured at a scale of greater than 1 g, greater than 2 g, greater than 3 g, greater than 4 g, greater than 5 g, greater than 6 g, greater than 7 g, greater than 8 g, greater than 9 g, greater than 10 g, greater than 15 g, greater than 20 g, or higher.

In some embodiments, RNA quality control may be performed and/or monitored at any time during production process of polyribonucleotides and/or compositions comprising the same. For example, in some embodiments, RNA quality control parameters, including one or more of RNA identity (e.g., sequence, length, and/or RNA natures), RNA integrity, RNA concentration, residual DNA template, and residual dsRNA, may be assessed and/or monitored after each or certain steps of a polyribonucleotide manufacturing process, e.g., after in vitro transcription, and/or each purification step.

In some embodiments, the stability of polyribonucleotides (e.g., produced by in vitro transcription) and/or compositions comprising polyribonucleotides can be assessed under various test storage conditions, for example, at room temperatures vs. fridge or sub-zero temperatures over a period of time (e.g., at least 3 months, at least 6 months, at least 9 months, at least 12 months, or longer). In some embodiments, polyribonucleotides (e.g., ones described herein) and/or compositions thereof may be stored stable at a fridge temperature (e.g., about 4° C. to about 10° C.) for at least 1 month or longer including, at least 2 months, at least 3 months, at least 4 months, at least 5 months, at least 6 months, at least 7 months, at least 8 months, at least 9 months, at least 10 months, at least 11 months, or at least 12 months or longer. In some embodiments, polyribonucleotides (e.g., ones described herein) and/or compositions thereof may be stored stable at a sub-zero temperature (e.g., −20° C. or below) for at least 1 month or longer including, at least 2 months, at least 3 months, at least 4 months, at least 5 months, at least 6 months, at least 7 months, at least 8 months, at least 9 months, at least 10 months, at least 11 months, or at least 12 months or longer. In some embodiments, polyribonucleotides (e.g., ones described herein) and/or compositions thereof may be stored stable at room temperature (e.g., at about 25° C.) for at least 1 month or longer.

In some embodiments, one or more assessments may be utilized during manufacture, or other preparation or use of polyribonucleotides (e.g., as a release test).

In some embodiments, one or more quality control parameters may be assessed to determine whether polyribonucleotides described herein meet or exceed acceptance criteria (e.g., for subsequent formulation and/or release for distribution). In some embodiments, such quality control parameters may include, but are not limited to RNA integrity, RNA concentration, residual DNA template and/or residual dsRNA. Certain methods for assessing RNA quality are known in the art; for example, one of skill in the art will recognize that in some embodiments, one or more analytical tests can be used for RNA quality assessment. Examples of such certain analytical tests may include but are not limited to gel electrophoresis, UV absorption, and/or PCR assay.

In some embodiments, a batch of polyribonucleotides may be assessed for one or more features as described herein to determine next action step(s). For example, a batch of polyribonucleotides can be designated for one or more further steps of manufacturing and/or formulation and/or distribution if RNA quality assessment indicates that such a batch of polyribonucleotides meet or exceed the relevant acceptance criteria. Otherwise, an alternative action can be taken (e.g., discarding the batch) if such a batch of polyribonucleotides does not meet or exceed the acceptance criteria.

In some embodiments, a batch of polyribonucleotides that satisfy assessment results can be utilized for one or more further steps of manufacturing and/or formulation and/or distribution.

### A. RNA Production

Those skilled in the art are aware of a variety of techniques that can be used to produce RNAs as described herein, including chemical or enzymatic (e.g., by polymerization) synthesis. In many embodiments, RNA is produced by transcription, e.g., by in vivo or in vitro transcription. Indeed, one advantage of RNA as an active agent for use in pharmaceutical compositions (e.g., immunogenic compositions, e.g., vaccines) or other therapeutic contexts is its facile production by in vitro transcription. Particularly given that relatively modest adjustments to manufacturing processes can often optimize production of related sequences, the present disclosure teaches that RNA modalities are particularly desirable for use as active agents in pharmaceutical compositions (e.g., immunogenic compositions, e.g., vaccines). Moreover, the present disclosure provides a particular insight that RNA is particularly useful as an active agent in HSV (e.g., HSV-1 and/or HSV-2) vaccines, as it permits facile adaptation (e.g., sequence alteration) to emerging or locally-relevant strains and/or antigens (e.g., permitting customization of antigen sequences in light of, for example, circulating strains and/or HLA allele diversity within relevant populations (e.g., in a particular geography/region). Furthermore, as the production of RNA requires only a single development and manufacturing platform, irrespective of the encoded pathogen antigens. Thus, RNA has the potential of rapid, cost-efficient, high-volume manufacturing and flexible stockpiling (long term storage of low-volume libraries of frozen plasmid and unformulated RNA, which can be rapidly formulated and distributed). Particularly for an HSV (e.g., HSV-1 and/or HSV-2) infection, where timing of administration (e.g., vaccine administration) relative to season and/or incidence of outbreak may materially impact effectiveness, such ability to store and promptly reconstitute may prove and important advantage with critical benefits relative to alternative strategies.

Typically, RNA is transcribed in vitro from a linearized (e.g., by restriction digestion) or amplified (e.g., PCR-amplified) DNA template. Those skilled in the art are aware of a variety of promoters useful for directing RNA synthesis by a transcription of a DNA template, for example by a DNA-dependent RNA polymerase such as, for example, T7, T3, SP6, or Syn5 RNA polymerase.

A typical in vitro transcription reaction will include a DNA template, rNTPs for the four bases (i.e., adenine, cytosine, guanine and uracil), optionally a cap analog, the relevant RNA polymerase, and appropriate buffers and/or salts. In some embodiments, one or more of a ribonuclease (RNase) inhibitor and/or a pyrophosphatase may be included.

In some embodiments, rNTPs utilized in an in vitro transcription reaction include one or more nucleotide analogs. In some embodiments, a nucleotide analog is 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-0-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-0-(1-thiophosphate)-adenosine, 5′-0-(1-thiophosphate)-cytidine, 5′-0-(1-thiophosphate)-guanosine, 5′-0-(1-thiophosphate)-uridine, 5′-0-(1-thiophosphate)-pseudouridine, 6-aza-cytidine, 2-thio-cytidine, alpha-thio-cytidine, Pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thio-adenosine, 8-azido-adenosine, 7-deaza-adenosine, pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine, or a combination thereof.

In some embodiments, uridine analog(s) are utilized. In some embodiments, no natural uridine is utilized. Thus, in some embodiments 100% of the uracil in the coding sequence have a chemical modification (relative to uridine); in may embodiments at 5-position of the uracil. In particular embodiments, pseudouridine is utilized.

In particular embodiments, utilized nucleotide analogs comprise pseudouridine, N1-methylpseudouridine, 5-methylcytosine, -methoxyuridine, and combinations thereof.

In some embodiments, the four rNTPs are utilized in equimolar concentrations in in vitro transcription reactions; in some embodiments, they are not equimolar. For example, in some embodiments, one or more rNTPs is at a relatively lower concentration and, in some embodiments, may be supplemented by one or more “feedings” over time during the reaction. In some particular embodiments, rGTP is fed over time (so that the IVT reaction is a “G fed batch” process). Alternatively or additionally, in some particular embodiments, rUTP is fed over time (so that the IVT reaction is a “U fed batch” or “G/U fed batch” process.

In some embodiments, one or more of rNTP concentration, salt concentration, metal concentration, pH, temperature etc, is adjusted for production of a particular RNA construct in order to optimize, for example, one or more of RNA integrity, capping efficiency, contaminant (e.g., dsRNA) level, intact transcript level (e.g., relative to template DNA concentration in the reaction), etc.

In some embodiments, exemplary reagents used in RNA in vitro transcription include: a DNA template (linearized plasmid DNA or PCR product) with a promoter sequence that has a high binding affinity for its respective RNA polymerase such as bacteriophage-encoded RNA polymerases (T7, T3, SP6, or Syn5); ribonucleotide triphosphates (NTPs) for the four bases (adenine, cytosine, guanine and uracil); optionally, a cap analogue as defined herein (e.g. m7G(5′)ppp(5′)G (m7G)); optionally, further modified nucleotides as defined herein; a DNA-dependent RNA polymerase capable of binding to the promoter sequence within the DNA template (e.g. T7, T3, SP6, or Syn5 RNA polymerase); optionally, a ribonuclease (RNase) inhibitor to inactivate any potentially contaminating RNase; optionally, a pyrophosphatase to degrade pyrophosphate, which may inhibit RNA in vitro transcription; MgCl2, which supplies Mg2+ ions as a co-factor for the polymerase; a buffer (TRIS or HEPES) to maintain a suitable pH value, which can also contain antioxidants (e.g. OTT), and/or polyamines such as spermidine at optimal concentrations, e.g. a buffer system comprising TRIS-Citrate as disclosed in WO2017/109161.

In the context of RNA production, in some embodiments, it may be desired to provide GMP-grade RNA. In some embodiments, GMP-grade RNA may be produced using a manufacturing process approved by regulatory authorities. In some embodiments, RNA production is performed under current good manufacturing practice (GMP), implementing various quality control steps on DNA and/or RNA level, for example, in some embodiments according to quality steps described in WO2016/180430. In some embodiments, RNA of the present disclosure is a GMP-grade RNA.

### X. DNA Constructs

Among other things, the present disclosure provides DNA constructs, for example that may encode one or more antibody agents as described herein, or components thereof. In some embodiments, DNA constructs provided by and/or utilized in accordance with the present disclosure are comprised in a vector.

Non-limiting examples of a vector include plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). In some embodiments, a vector is an expression vector. In some embodiments, a vector is a cloning vector. In general, a vector is a nucleic acid construct that can receive or otherwise become linked to a nucleic acid element of interest (e.g., a construct that is or encodes a payload, or that imparts a particular functionality, etc.)

Expression vectors, which may be plasmid or viral or other vectors, typically include an expressible sequence of interest (e.g., a coding sequence) that is functionally linked with one or more control elements (e.g., promoters, enhancers, transcription terminators, etc.). Typically, such control elements are selected for expression in a system of interest. In some embodiments, a system is ex vivo (e.g., an in vitro transcription system); in some embodiments, a system is in vivo (e.g., a bacterial, yeast, plant, insect, fish, vertebrate, mammalian cell or tissue, etc.).

Cloning vectors are generally used to modify, engineer, and/or duplicate (e.g., by replication in vivo, for example in a simple system such as bacteria or yeast, or in vitro, such as by amplification such as polymerase chain reaction or other amplification process). In some embodiments, a cloning vector may lack expression signals.

In many embodiments, a vector may include replication elements such as primer binding site(s) and/or origin(s) of replication. In many embodiments, a vector may include insertion or modification sites such as restriction endonuclease recognition sites and/or guide RNA binding sites, etc.

In some embodiments, a vector is a viral vector (e.g., an AAV vector). In some embodiments, a vector is a non-viral vector. In some embodiments, a vector is a plasmid.

Those skilled in the art are aware of a variety of technologies useful for the production of recombinant polynucleotides (e.g., DNA or RNA) as described herein. For example, restriction digestion, reverse transcription, amplification (e.g., by polymerase chain reaction), Gibson assembly, etc., are well established and useful tools and technologies. Alternatively or additionally, certain nucleic acids may be prepared or assembled by chemical and/or enzymatic synthesis. In some embodiments, a combination of known methods is utilized to prepare a recombinant polynucleotide.

In some embodiments, polynucleotide(s) of the present disclosure are included in a DNA construct (e.g., a vector) amenable to transcription and/or translation.

In some embodiments, a construct of an expression vector comprises a polynucleotide that encodes proteins and/or polypeptides of the present disclosure operatively linked to a sequence or sequences that control expression (e.g., promoters, start signals, stop signals, polyadenylation signals, activators, repressors, etc.). In some embodiments, a sequence or sequences that control expression are selected to achieve a desired level of expression. In some embodiments, more than one sequence that controls expression (e.g., promoters) are utilized. In some embodiments, more than one sequence that controls expression (e.g., promoters) are utilized to achieve a desired level of expression of a plurality of polynucleotides that encode a plurality proteins and/or polypeptides. In some embodiments, a plurality of recombinant proteins and/or polypeptides are expressed from the same vector (e.g., a bi-cistronic vector, a tri-cistronic vector, multi-cistronic). In some embodiments, a plurality of polypeptides are expressed, each of which is expressed from a separate vector.

In some embodiments, an expression vector comprising a polynucleotide of the present disclosure is used to produce a RNA and/or protein and/or polypeptide in a host cell. In some embodiments, a host cell may be in vitro (e.g., a cell line)—for example a cell or cell line (e.g., Human Embryonic Kidney (HEK cells), Chinese Hamster Ovary cells, etc.) suitable for producing polynucleotides of the present disclosure and proteins and/or polypeptides encoded by said polynucleotides.

In some embodiments, an expression vector is an RNA expression vector. In some embodiments, an RNA expression vector comprises a polynucleotide template used to produce a RNA in cell-free enzymatic mix. In some embodiments, an RNA expression vector comprising a polynucleotide template is enzymatically linearized prior to in vitro transcription. In some embodiments, a polynucleotide template is generated through PCR as a linear polynucleotide template. In some embodiments, a linearized polynucleotide is mixed with enzymes suitable for RNA synthesis, RNA capping and/or purification. In some embodiments, the resulting RNA is suitable for producing proteins encoded by the RNA.

A variety of methods are known in the art to introduce an expression vector into host cells. In some embodiments, a vector may be introduced into host cells using transfection. In some embodiments, transfection is completed, for example, using calcium phosphate transfection, lipofection, or polyethylenimine-mediated transfection. In some embodiments, a vector may be introduced into a host cell using transduction.

In some embodiments, transformed host cells are cultured following introduction of a vector into a host cell to allow for expression of said recombinant polynucleotides. In some embodiments, a transformed host cells are cultured for at least 12 hours, 16 hours, 20 hours, 24 hours, 28 hours, 32 hours, 36 hours 40 hours, 44 hours, 48 hours, 52 hours, 56 hours, 60 hours, 64 hours, 68 hours, 72 hours or longer. Transformed host cells are cultured in growth conditions (e.g., temperature, carbon-dioxide levels, growth medium) in accordance with the requirements of a host cell selected. A skilled artisan would recognize culture conditions for host cells selected are well known in the art.

### EXEMPLIFICATION

### Example 1: Identification of Immunogenic T Cell Antigens

The present Examples includes, among other things, results of systematic analyses that produced an unexpected list or set of T cell antigens (e.g., CD4 and/or CD8 T cell antigens) for HSV. T cell antigens of the present Examples are useful, individually or in combination, for the production of HSV antigen constructs (e.g., for use in HSV vaccine agents). The present Example provides, among other things, discussion of the systematic analysis applied, which included selection of source data and stepwise evaluations of source data to generate an unpredicted list of T cell antigens for HSV.

A first step in the systematic identification of T cell antigens for HSV included analysis of publicly available datasets relating to T cell induction in HSV-infected individuals. T cell antigens for HSV preferably demonstrate immunogenicity as a target of T cells in HSV-infected human subjects, and still more preferably elicit T cell responses in a large proportion of infected human subjects in cohort studies. Many studies of T cell responses to HSV have been conducted. However, the present inventors appreciate that many studies are narrow (e.g., explored immune responses to one or a few antigens at a time in animal models or human subjects) or subject to bias. Comprehensive evaluation of every HSV gene with minimal bias is difficult to achieve by combining data from independent studies narrowly focused on a few targets. Moreover, systematic analysis of data from diverse studies is challenging due to use of unique methodologies, reagents and patient cohorts by different groups. The analysis of the present Example therefore included selection of studies that measured T cell responses (e.g., CD8 T cell responses) to the majority of HSV proteins simultaneously in multiple subjects. Three publications met these criteria and were further evaluated as sources of pan-genome immunogenicity data for HSV T cell targets: Hosken (J Virol 2006 June; 80(11):5509-152006-15; PMID: 16699031; hereafter “Hosken” or “Hosken 2006”), Jing (J Clin Invest 2012 February; 122(2):654-73; PMID: 22214845; hereafter “Jing” or “Jing 2012”), and Long (Virology 2014 September; 464-465:296-311; PMID: 25108380; hereafter “Long” or “Long 2014”).

Having identified the above sources of pan-genome immunogenicity data for HSV T cell targets, the next step was to determine how to weight the three studies. To do so, we first determined if the reported frequencies of individuals with T cell responses (percent responders) to each antigen correlated between studies. We assumed that broad trends in immunogenicity should correspond between studies, and a lack of correlation would suggest possible methodological deficits. A strong correlation (r=0.70, Spearman correlation) was observed between the percent responders reported in Jing 2012 and Hosken 2006, but no significant correlation was observed between the results in Long 2014 and either other dataset. We therefore deemed Long 2014 to be a low reliability dataset and excluded it from further analysis.

Selection of T cell antigens for HSV based on the studies of Hosken 2006 and Jing 2012, from the set of proteins encoded by genes of the HSV genome, was undertaken in two steps. Each step was designed to reflect the form of data available in the respective publications. First, all HSV proteins below the median T cell percent responder frequency in Hosken 2006 were eliminated from consideration as T cell antigens (except that, as certain HSV genes were not represented in the data from Hosken 2006, genes for which no data were available were not eliminated from consideration). Second, genes for which fewer than 3/7 subjects had measurable T cell responses in Jing 2012 were eliminated from consideration as T cell antigens. A total of 14 genes remained after these elimination steps, which were further refined based on expression level as described below.

A further analysis was conducted to identify candidate T cell antigens characterized by expression above a median level. In this further analysis, RNA and protein expression studies were evaluated to identify HSV genes encoding the most highly expressed T cell antigens during early viral replication. For this analysis, study data were derived from multiple sources.

Some of the data sources comprised scRNA-Seq data. For these studies, the gene expression profiles of individual cells were analyzed, and by quantifying the expression of well-characterized immediate early (IE) genes (US1, UL54, UL50, UL23, UL30) and late genes (UL48, UL45, UL44, US11, UL36), the cells were placed in an approximate temporal ordering from early activation too late. Specifically, expression of late genes was averaged (termed as ‘late gene score’) for each single cell, and cells were arranged in order of increasing late gene score. Cells were then divided into 5 equal bins, and expression profiles of cells belonging to the same bin were averaged to generate a 5 distinct expression profiles representing different timepoints along the activation continuum.

All expression profiles (from bulk RNA-Seq, scRNA-Seq, and proteomics) were normalized by scaling each sample/stage to have a maximum expression of 1 and minimum expression of 0. The resulting normalized profiles were used for visualizing overall expression and calculating Spearman's correlation coefficient between pairs of expression profiles. The expression profiles were also sorted according to the expression of well-characterized immediate early (IE) genes (US1, UL54, UL50, UL23, UL30) and late genes (UL48, UL45, UL44, US11, UL36) to help determine which profiles more likely represented early stages of activation. Specifically, expression of late genes was averaged (termed as ‘late gene score’) for each single cell and cells were arranged in order of increasing late gene score (in precisely the manner used to order singles cells in the scRNA-Seq data analysis). By jointly analyzing the correlation coefficients and the “late gene score”, we selected 12 expression profiles from 11 studies that appeared to reliably represent expression during early activation. For each gene, the median expression level was determined across the 12 studies. Finally, across all genes, median expression was calculated, and genes with expression above the median were considered for vaccine inclusion. Based upon these analyses, a set of 10 T cell antigens (e.g., CD4 and/or CD8 T cell antigens) for HSV were identified (shown in Table 4).

Source literature is listed below with brief comments on methodology:
Fox (mBio. 2017 May-June; 8(3): e00745-17; PMID: 28611249)
   Data relating to HSV1 KOS in MRC5 fibroblasts, MOI of 5, from an expression table containing raw reads per gene (supplementary files in GSE109420) was normalized by number of reads per sample and by gene length. We took 4 hpi sample for downstream analysis.
Krenn (Cell Stem Cell. 2021 Aug. 5; 28(8):1362-1379.e7; PMID: 33838105)
   Data relate to RNA-seq on HSV1/mock infected hIPSC-derived 3D-brain organoid with or without IFNa2 treatment (which rescues HSV1 infection). Condition-specific sample subsets (designated by their SRR IDs and condition labels in the sheet) were acquired from GEO (accession ID: GSE145496). These samples were aligned to the concatenated GRCh38 and HSV-1 strain F genome using the STAR alignment (-runMode alignReads-outSAMattributes All-outSAMtype BAM-outMultimapperOrder Random). Reads overlapping HSV-1 genes were then aggregated using HTSeq package and HSV1 strain F reference annotation (HTSeq parameters: -t CDS-i gene). Gene-specific feature counts were TPM-normalized.
Wang (Virol J. 2020 Jul. 8; 17(1):95; PMID: 32641145)
   Data relate to Bulk RNA-seq on trigeminal ganglia of mouse and tree shrew, with HSV-1. Aligned reads from HSV-1 in BAM format were acquired from BIG Data center (accession number CRA001750) and quantified using featureCount function from Rsubread R package. Reads were normalized by gene length. We took 7 dpi time point samples for mouse and tree shrew.
Walter (2021. Herpesviral induction of germline transcription factor DUX4 is critical for viral gene expression. bioRxiv doi: 10.1101/202103.24.436599)
   Data relate to RNA-seq from HSV-1 infected 293T cells with and without DUX4 KO. HSV-1 gene-level counts across the WT and DUX4KO conditions at 18 hpi were downloaded using the GEO (GEO accession: GSE174759). Gene RS1-r had two rows with similar counts profile across the samples and was de-duplicated. The counts were then normalized by the total library size for the sample and scaled by 10\hat{ }4. We took 18 hpi time point for downstream analysis.
Tokuyama (2021. Endogenous retroviruses mediate IFN-independent protection against HSV-2 infection. Query DataSets for GSE185281 available at NCBI Gene Expression Ominibus (GEO))
   Data relate to RNA-seq from HSV-2 infected mice (vaginal samples). Raw read counts for two WT B6 mice infected with HSV-2 were downloaded from SRA archive under PRJNA768446. Reads were aligned to a concatenated human (hg38) and HSV-2 (HGS2) genome using the STAR software, and HSV-2 gene expression was quantified using htseq-count. Data is shown as average of log 2(counts/total library size*1E4+1) values from 2 replicates.
Khoury-Hanold (Cell Host Microbe. 2016 Jun. 8; 19(6):788-99; PMID: 27281569)
   Data relate to bulk RNA-seq from in vivo HSV-1 infection: vaginal route, DRG (dorsal root ganglia) and LIM (large intestinal musculara) sequences. Processed and normalized expression table was acquired from GSE74215. For downstream analysis, expression was additionally scaled to account for gene length bias.
Tombácz (Front Microbiol. 2017 Jun. 20; 8:1079; PMID: 28676792)
   Data relate to Pacbio seq of kidney epithelial cells infected with HSV1 (MOI=1). Expression data was extracted from Supplementary Files in the GEO record GSE97785 and gene names were unified using NCBI nomenclature. Data is in the form of percentage of HSV2 gene reads in each sample, and those mapping to the same gene are aggregated.
Wyler (Nature Communications volume 10, Article number: 4878 (2019); PMID: 31653857)
   Data relate to scRNA-seq (host+viral) of HSV-1 infected fibroblasts GSE123782. Read counts mapping to host and viral genes were obtained from supplementary files in the publication. To generate expression profiles for samples from each time point, reads from cells belonging to the same time point were aggregated and transcripts per million (TPM) values are calculated for each HSV-1 gene.
Drayman (Elife. 2019 May 15; 8:e46339. doi: 10.7554/eLife.46339; PMID 31090537)
   Data relate to scRNA-seq from HSV-1 infected fibroblasts, MOI2, 5 hpi. Read counts were obtained from GSE126042. Reads were normalized by the depth of each sample, multiplied by 10000 and log 2-normalized. Only cells with more than 50 reads aligned to HSV genome were considered HSV1+ and used for the analysis.
Kulej (Mol Cell Proteomics. 2017 April; 16(4 suppl 1):S92-S107; PMID: 28179408)
   Proteomics expression data generated from primary human foreskin fibroblast (HFF) cells infected with HSV-1 strain 17 syn+ was extracted from Supplementary Table S2A from Kulej et al., MCP 2017; 16(4 suppl 1):S92-S107, doi:10.1074/mcp.M116.065987. We took 6 hpi timepoint for downstream analysis.
Soh (Cell Rep. 2020 Oct. 6; 33(1):108235; PMID: 33027661)
   Proteomics expression data generated from human keratinocyte cell line (HaCaT) cells infected with HSV-1 KOS strain was extracted from Supplementary Table S1 from Soh et al., Cell Reports 2020; 33 (1), doi.org/10.1016/j.celrep.2020.108235. We took4 hpi timepoint for downstream analysis.
Saiz-Ros (Cells. 2019 Feb. 3; 8(2):120; PMID: 30717447)
   Proteomics expression data generated from HeLa cells infected with HSV-1 strain 17+ was extracted from Table S1 from Saiz-Ros et al., Cells. 2019 Feb. 3; 8(2):120, doi: 10.3390/cells8020120. We took 8 hpi timepoint for downstream analysis.

Additional T cell antigens for HSV were identified from inspection of source literature, which additional antigens could be used individually or in combination with others described herein. One group of additional antigens was selected from subunit vaccines that have progressed to clinical trials (Krishnan and Stuart. Front Microbiol. 2021 Dec. 7; 12:798927; PMID: 34950127). Another group of additional antigens was selected from promising prophylactic vaccination data in small animal models of HSV-2 infection (Morello et al. J Virol. 2011 April; 85(7):3461-72; PMID: 21270160; Platt et al. Cells. 2013 Jan. 4; 2(1):19-42; PMID: 24709642). A third group of additional antigens that were identified as candidate T cell antigens for HSV from Hosken 2006 and Jing 2012 (see above in this Example) but did not meet the expression threshold discussed above. These analyses inspecting source literature produced a set of 9 additional T cell antigens (e.g., CD4 and/or CD8 T cell antigens) for HSV (shown in Table 5).

### Example 2: Exemplary RNA Constructs Encoding HSV Antigens

The present example describes certain exemplary HSV antigens, and sequences encoding them, that may be utilized in certain embodiments of the present disclosure. Exemplary HSV (e.g., HSV-1 and/or HSV-2) antigens can be found in Tables 3-5.

In some particular embodiments, an administered RNA has a structure:

Structure 1: m₂^{7,3′-O}Gppp(m₁^{2′-O})ApG-hAg-Kozak-SEC-Immunogen-FI-A30L70, wherein m₂^{7,3′-O}Gppp(m₁^{2′-O})ApG=5′ cap; hAg=5′ UTR human alpha-globin; SEC=signal peptide (SP); Immunogen=a nucleotide sequence comprising a sequence that encodes an antigen described herein; FI=a 3′-UTR that is or comprises a sequence (e.g., 3′ UTR) from the “amino terminal enhancer of split” (AES) messenger RNA and a sequence (e.g., a non-coding region) from the mitochondrial encoded 12S ribosomal RNA (MT-RNR1); and A30L70=a polyA sequence comprising 30 adenine nucleotides followed by 70 adenine nucleotides, wherein the 30 adenine nucleotides and 70 adenine nucleotides are separated by a linker sequence.

In some embodiments, an administered RNA has a structure:

Structure 2: m₂^{7,3′-O}Gppp(m₁^{2′-O})ApG-hAg-Kozak-SEC-Immunogen-MITD-FI-A30L70, wherein m₂^{7,3-O}Gppp(m₁^{2′-O})ApG=5′ cap; hAg=5′ UTR human alpha-globin; SEC=signal peptide (SP); Immunogen=a nucleotide sequence comprising a sequence that encodes one or more antigens described herein; MITD=MHC Class I trafficking signal (MITD); FI=a 3′-UTR that is or comprises a sequence (e.g., 3′ UTR) from the “amino terminal enhancer of split” (AES) messenger RNA and a sequence (e.g., a non-coding region) from the mitochondrial encoded 12S ribosomal RNA (MT-RNR1); and A30L70=a polyA sequence comprising 30 adenine nucleotides followed by 70 adenine nucleotides, wherein the 30 adenine nucleotides and 70 adenine nucleotides are separated by a linker sequence.

In some embodiments, an Immunogen sequence encodes a plurality of immunogenic fragments (e.g., comprising epitopes) from an antigen. In some embodiments, an Immunogen sequence encodes a plurality of immunogenic fragments (e.g., epitopes) from two or more antigens. In some embodiments. In some embodiments, such immunogenic fragments are linked together to form an Immunogen sequence by linkers (e.g., in some embodiments a linker that is enriched in G and/or S amino acid residues). In some embodiments, a linker may be or comprise an amino acid sequence of GGSGGGGSGG (SEQ ID NO: 165). In some embodiments, a linker may be or comprise an amino acid sequence of GGSLGGGGSG (SEQ ID NO: 166).

### Example 3: Exemplary RNA Constructs Encoding Multiepitope HSV Antigens

The present example describes certain exemplary HSV multiepitope antigens, and sequences encoding them, that may be utilized in certain embodiments of the present disclosure.

Exemplary Construct Encoding a HSV multi-epitope polypeptide:

Structure m₂^{7,3′-O}Gppp(m₁^{2′-O})ApG-hAg-Kozak-SEC-CD8 string-MITD-FI-A30L70

In some embodiments, a T cell antigen string can include at least 2 (including, e.g., at least 1, at least 2, at least 3, at least 4, at least 5, at least 10, at least 15, at least 20, or more) T cell epitopes and/or HLA-I epitopes as listed in Tables 3-5.

### Example 4: Exemplary Prediction and/or Characterization of MHC Presentation

The present Example describes an exemplary approach to assessing MHC presentation which may be used in accordance with the present disclosure to select and/or characterize antigenic peptides as described herein.

In some embodiments, an antigenic peptide is selected and/or characterized through analysis of its amino acid sequence using an MHC-peptide presentation prediction algorithm or MHC-peptide presentation predictor, for example implemented in a computer processor (e.g., a computer processor that has been trained by a machine learning software), which determines a likelihood of binding and presentation of an peptide by an MHC class I or an MHC class II antigen.

In some embodiments, an MHC-peptide presentation prediction algorithm or MHC-peptide presentation predictor is or comprises neonmhc 1 and/or neonmhc2, which predict and/or characterize likelihood of MHC class I and MHC class II binding, respectively. Alternatively or additionally, in some embodiments, an MHC-peptide presentation prediction algorithm or MHC-peptide presentation predictor is or comprises NetMHCpan or NetMHCIIpan. In some embodiments, a hidden markov model approach may be utilized for MHC-peptide presentation prediction and/or characterization. In some embodiments, the peptide prediction model MARIA may be utilized. In some embodiments, NetMHCpan is not utilized to predict or characterize likelihood of MHC binding for peptides as described herein. In some embodiments, the peptide prediction model MARIA may be utilized. In some embodiments, NetMHCIIpan is not utilized to predict or characterize likelihood of MHC binding for peptides as described herein. In some embodiments, neither NetMHCpan nor NetMHCIIpan is utilized to predict or characterize likelihood of MHC binding for peptides as described herein. In some embodiments, an MHC-peptide presentation prediction algorithm or MHC-peptide presentation predictor is or comprises RECON, which offers high quality MHC-peptide presentation prediction based on expression, processing and binding capabilities.

In some embodiments, multiple MHC-peptide presentation prediction algorithms or MHC-peptide presentation predictors may be utilized; in some such embodiments, results obtained with different strategies are compared with one another. In some embodiments, a determination that a particular peptide is likely to be or is significantly likely to be presented by MHC class I or MHC class II may be considered to be better established if two or more algorithms or predictors agree.

Alternatively or additionally, identification and/or characterization of MHC binding (e.g., of MHC class I and/or MHC class II binding) may involve experimental assessment, or reports thereof, which may involve presentation in one or more in vitro systems and/or in one or more organisms. In some embodiments, such assessment utilizes mammalian cells or systems; in some embodiments such assessment utilizes primate (e.g., in some embodiments, human and/or in some embodiments, non-human primate) cells or systems.

### Example 5: Exemplary HLA Class I and Class II Binding Assays

The present Example describes exemplary techniques for assessing peptide binding to HLA molecules. In some embodiments, exemplified technologies may determine and/or characterize (e.g., quantify) binding affinities for HLA class I and HLA class II.

In general, binding assays can be performed with peptides that are either motif-bearing or not motif-bearing. A detailed description of an exemplary protocol that can be utilized to measure the binding of peptides to Class I and Class II MHC has been published (Sette et al., Mol. Immunol. 31:813, 1994; Sidney et al., in Current Protocols in Immunology, Margulies, Ed., John Wiley & Sons, New York, Section 18.3, 1998). Briefly, purified MHC molecules (5 to 500 nM) are incubated with various unlabeled peptide inhibitors and 1-10 nM ¹²⁵l-radiolabeled probe peptides for 48 h in PBS containing 0.05% Nonidet P40 (NP40) (or 20% w/v digitonin for H-2 IA assays) in the presence of a protease inhibitor cocktail. Assays are typically performed at pH 7.0, though in some embodiments a lower pH (typically above about pH 4.0) may be performed.

Following incubation, MHC-peptide complexes are separated from free peptide, for example by gel filtration, e.g., on 7.8 mm×15 cm TSK200 columns (TosoHaas 16215, Montgomeryville, PA), though those skilled in the art will appreciate that column size can be adjusted, if desired, for example to improve separation of bound vs unbound peptides of a particular size or characteristic. The eluate from the TSK columns is passed through a Beckman 170 radioisotope detector, and radioactivity is plotted and integrated using a Hewlett-Packard 3396A integrator, and the fraction of peptide bound is determined.

Radiolabeled peptides can be iodinated using the chloramine-T method. Typically, in preliminary experiments, each MHC preparation is titered in the presence of fixed amounts of radiolabeled peptides to determine the concentration of HLA molecules necessary to bind 10-20% of the total radioactivity. Subsequent inhibition and direct binding assays can be performed using these HLA concentrations.

Since under these conditions [label]<[HLA] and IC₅₀>[HLA], the measured IC₅₀ values are often reasonable approximations of the true K_{D} values. Peptide inhibitors are typically tested at concentrations ranging from 120 μg/ml to 1.2 ng/ml, and are tested in two to four completely independent experiments. To allow comparison of the data obtained in different experiments, a relative binding figure is typically calculated for each peptide by dividing the IC₅₀ of a positive control for inhibition by the IC₅₀ for each tested peptide (typically unlabeled versions of the radiolabeled probe peptide). For database purposes, and inter-experiment comparisons, relative binding values can be compiled. Such values can subsequently be converted back into IC₅₀ nM values, for example by dividing the IC₅₀ nM of the positive controls for inhibition by the relative binding of the peptide of interest. This method of data compilation has proven to provide accurate and consistent comparison for peptides that have been tested on different days, or with different lots of purified MHC.

Alternatively or additionally, live cell/flow cytometry-based assays can also be used. This is a well-established assay utilizing the TAP-deficient hybridoma cell line T2 (American Type Culture Collection (ATCC Accession No. CRL-1992), Manassas, Va.). The TAP deficiency in this cell line leads to inefficient loading of MHCI in the ER and an excess of empty MHCIs. Salter and Cresswell, EMBO J. 5:94349 (1986); Salter, Immunogenetics 21:235-46 (1985). Empty MHCIs are highly unstable, and are therefore short-lived. When T2 cells are cultured at reduced temperatures, empty MHCIs appear transiently on the cell surface, where they can be stabilized by the exogenous addition of MHCI-binding peptides. To perform this binding assay, peptide-receptive MHCIs are induced by culturing aliquots of 107 T2 cells overnight at 26° C. in serum free AIM-V medium alone, or in medium containing escalating concentrations (0.1 to 100 M) of peptide. Cells are then washed twice with PBS, and subsequently incubated with a fluorescent tagged HLA allele-specific monoclonal antibody (e.g., HLA-A02:01-specific monoclonal antibody, BB7.2), to quantify cell surface expression. Samples are acquired on a FACS Calibur instrument (Becton Dickinson) and the mean fluorescence intensity (MFI) determined using the accompanying Cellquest software.

### Example 6: Confirmation of Immunogenicity

The present Example describes an exemplary method for confirmation of immunogenicity, in particular by utilizing in vitro expansion (IVE) assays to test the ability of one or more antigens or peptides to expand CD8+ T cells.

Mature professional APCs are prepared for these assays in the following way. 80-90×10⁶ PBMCs from a healthy human donor are plated in 20 ml of RPMI media containing 2% human AB serum, and incubated at 37° C. for 2 hours to allow for plastic adherence by monocytes. Non-adherent cells are removed and the adherent cells are cultured in RPMI, 2% human AB serum, 800 IU/ml of GM-CSF and 500 IU/ml of IL-4. After 6 days, TNF-alpha is added to a final concentration of 10 ng/ml. On day 7, the dendritic cells (DC) are matured either by the addition of 12.5 mg/ml poly I:C or 0.3 μg/ml of CD4OL. The mature dendritic cells (mDC) are harvested on day 8, washed, and either used directly or cryopreserved for future use.

For the IVE of CD8+ T cells, aliquots of 2×10⁵ mDCs are pulsed with each peptide at a final concentration of 100 micromole, incubated for 4 hours at 37° C., and then irradiated (2500 rads). The peptide-pulsed mDCs are washed twice in RPMI containing 2% human AB serum. 2×10⁵ mDCs and 2×10⁶ autologous CD8+ cells are plated per well of a 24-well plate in 2 ml of RPMI containing 2% human AB, 20 ng/ml IL-7 and 100 μg/ml of IL-12, and incubated for 12 days. The CD8+ T cells are then re-stimulated with peptide-pulsed, irradiated mDCs. Two to three days later, 20 IU/ml IL-2 and 20 ng/IL7 are added. Expanding CD8+ T cells are re-stimulated every 8-10 days, and are maintained in media containing IL-2 and IL-7. Cultures are monitored for peptide-specific T cells using a combination of functional assays and/or tetramer staining. Parallel IVES with the modified and parent peptides allowed for comparisons of the relative efficiency with which the peptides expanded peptide-specific T cells.

### Example 7: Quantitative and Functional Assessment of CD8+ and CD4+ T Cells Tetramer Staining

MHC tetramers are purchased or manufactured on-site, and are used to measure peptide-specific T cell expansion in the IVE assays. For the assessment, tetramer is added to 1×10⁵ cells in PBS containing 1% FCS and 0.1% sodium azide (FACS buffer) according to manufacturer's instructions. Cells are incubated in the dark for 20 minutes at room temperature. Antibodies specific for T cell markers, such as CD8, are then added to a final concentration suggested by the manufacturer, and the cells are incubated in the dark at 4° C. for 20 minutes. Cells are washed with cold FACS buffer and resuspended in buffer containing 1% formaldehyde. Cells are acquired on a FACS Calibur (Becton Dickinson) instrument, and are analyzed by use of Cellquest software (Becton Dickinson). For analysis of tetramer positive cells, the lymphocyte gate is taken from the forward and side-scatter plots. Data are reported as the percentage of cells that are CD8+/Tetramer+.

CD4⁺ T cell responses towards antigens or peptides can be tested using the ex vivo induction protocol. In this example, CD4+ T cell responses are identified by monitoring IFNγ and/or TNFα production in an antigen specific manner.

### Evaluation of Antigen Presentation:

For a subset of antigens or peptides (e.g., that are or comprise predicted or selected epitope(s) as described herein), affinity for the indicated HLA alleles and/or stability with the HLA alleles can be determined.

An exemplary detailed description of a protocol that can be utilized to measure the binding affinity of peptides to Class I MHC has been published (Sette et al, Mol. Immunol. 31(11):813-22, 1994). In brief, MHCI complexes are prepared and bound to radiolabeled reference peptides. Peptides are incubated at varying concentrations with these complexes for 2 days, and the amount of remaining radiolabeled peptide bound to MHCI is measured using size exclusion gel-filtration. The lower the concentration of test peptide needed to displace the reference radiolabeled peptide demonstrates a stronger affinity of the peptide for MHCI. Peptides with affinities to MHCI<50 nM are generally considered strong binders while those with affinities <150 nM are considered intermediate binders and those <500 nM are considered weak binders (Fritsch et al, 2014).

An exemplary detailed description of a protocol that can be utilized to measure binding stability of peptides to Class I MHC has been published (Harndahl et al. J Immunol Methods. 374:5-12, 2011). Briefly, synthetic genes encoding biotinylated MHC-I heavy and light chains are expressed in E. coli and purified from inclusion bodies using standard methods. The light chain (β2m) is radio-labeled with iodine (125I), and combined with the purified MHC-I heavy chain and peptide of interest at 18° C. to initiate pMHC-I complex formation. These reactions are carried out in streptavidin coated microplates to bind the biotinylated MHC-I heavy chains to the surface and allow measurement of radiolabeled light chain to monitor complex formation. Dissociation is initiated by addition of higher concentrations of unlabeled light-chain and incubation at 37° C. Stability is defined as the length of time in hours it takes for half of the complexes to dissociate, as measured by scintillation counts. MHC-II binding affinity with peptides is measured following the same general procedure as with measuring MHCI-peptide binding affinity. Prediction algorithms utilized for predicting MHCII alleles for binding to a given peptide are described herein. Alternatively or additionally, NetMHCIIpan may be utilized for prediction of binding.

To assess whether particular antigens or peptides or epitopes could be processed and presented from a larger polypeptide context, peptides eluted from HLA (class I or class II) molecules isolated from cells expressing the genes of interest can be analyzed by tandem mass spectrometry (MS/MS).

### ELISPOT

Peptide-specific T cells are functionally enumerated, for example, using the ELISPOT assay (BD Biosciences), which measures the release of IFNgamma from T cells on a single cell basis. Target cells (T2 or specific HLA-transfected C1Rs (e.g., HLA-A0201 transfected C1Rs)) are pulsed with 10 uM peptide for 1 hour at 37° C., and washed three times. 1×105 peptide-pulsed targets are co-cultured in the ELISPOT plate wells with varying concentrations of T cells (5×102 to 2×103) taken from the IVE culture. Plates are developed according to the manufacturer's protocol, and analyzed on an ELISPOT reader (Cellular Technology Ltd.) with accompanying software. Spots corresponding to the number of IFNgamma-producing T cells are reported as the absolute number of spots per number of T cells plated. T cells expanded on modified peptides are tested not only for their ability to recognize targets pulsed with the modified peptide, but also for their ability to recognize targets pulsed with the parent peptide.

### CD107 Staining

CD107a and b are expressed on the cell surface of CD8+ T cells following activation with cognate peptide. The lytic granules of T cells have a lipid bilayer that contains lysosomal-associated membrane glycoproteins (“LAMPs”), which include the molecules CD107a and b. Without wishing to be bound by any one theory, it is proposed that, when cytotoxic T cells are activated through the T cell receptor, the membranes of these lytic granules mobilize and fuse with the plasma membrane of the T cell. The granule contents are released, and this leads to the death of the target cell. As the granule membrane fuses with the plasma membrane, C107a and b are exposed on the cell surface, and therefore are markers of degranulation. Because degranulation as measured by CD107 a and b staining is reported on a single cell basis, the exemplary assay is used to functionally enumerate peptide-specific T cells. To perform the assay, peptide is added to specific HLA-transfected cells C1Rs (e.g., HLA-A0201 transfected C1Rs) to a final concentration of 20 M, the cells are incubated for 1 hour at 37° C., and washed three times. 1×105 of the peptide-pulsed C1R cells are aliquoted into tubes, and antibodies specific for CD107 a and b are added to a final concentration suggested by the manufacturer (Becton Dickinson). Antibodies are added prior to the addition of T cells in order to “capture” the CD107 molecules as they transiently appear on the surface during the course of the assay. 1×105 T cells from the culture are added next, and the samples are incubated for 4 hours at 37° C. The T cells are further stained for additional cell surface molecules such as CD8 and acquired on a FACS Calibur instrument (Becton Dickinson). Data is analyzed using the accompanying Cellquest software, and results are reported as the percentage of CD8+CD107 a and b+ cells.

### CTL Lysis

Cytotoxic activity can be measured, for example, using a chromium release assay. Target T2 cells are labeled for 1 hour at 37° C. with Na⁵¹Cr and washed 5×10³ target T2 cells are then added to varying numbers of T cells from the IVE culture. Chromium release is measured in supernatant harvested after 4 hours of incubation at 37° C. The percentage of specific lysis is calculated as:

Experimental release-spontaneous release/Total release-spontaneous release×100.

### Example 8: Administration of Polyepitopic Compositions

The present Example describes exemplary administration of compositions that comprise or deliver a plurality of epitopes.

For example, a polyepitopic vaccine (e.g., that comprises or delivers a collection of epitopes—e.g., as individual discrete peptides or as one or more polyepitopic peptides such as one or more string constructs as described herein).

In some embodiments, a polyepitopic vaccine comprises or delivers multiple cytotoxic T lymphocyte (CTL) and/or helper T lymphocyte (HTL) epitopes. In some embodiments, such a vaccine is administered to subject(s) at risk of or having experienced exposure to infection.

In some embodiments, a polyepitopic vaccine as described herein comprises or delivers one or more polypeptides, each of which encompasses multiple epitopes. In some embodiments, one or more monoepitopic peptides or polyepitopic antigens is delivered to a subject by administration of one or more a nucleic acid (e.g., DNA or and RNA) constructs. In some embodiments, a single nucleic acid construct (e.g., a DNA or RNA encoding a polyepitopic antigen) is administered. In some embodiments, a plurality of nucleic acids (e.g., each encoding a different monoepitopic or polyepitopic antigens) is administered. In some embodiments, an administered nucleic acid is an RNA (e.g., an mRNA); in some embodiments, a nucleic acid (e.g., an RNA) is administered in an LNP composition.

In some embodiments, an administered composition includes an aqueous carrier and/or alum.

In some embodiments, an initial administration is followed by one or more booster doses. In some embodiments, a booster dose includes the same amount of polyepitopic construct as the initial dose. In some embodiments, a booster dose include more or less of a polyepitopic construct than was provided in the initial dose. In some embodiments, a booster dose is administered after an interval of at least 1, 2, 3, 4, 5, 6, 7, 8, 9, 12, 11, 12, 13, 14, 15, 16, 17, 18, 29, 20, 21, 22, 23, 24 weeks or more after an initial dose. In some embodiments, multiple booster doses are administered. In some embodiments, each subsequent booster is administered at an interval that is the same as or longer than that between its immediate predecessor dose and the dose before that. In some embodiments, 2, 3, 4, 5, 6, or more boosters are administered. In some embodiments, not more than 4 doses total are administered. In some embodiments, not more than 3 doses total are administered. In some embodiments, not more than two doses total are administered. In some embodiments, not more than 1, 2, 3 or 4 doses are administered within a particular 12 month period. In some embodiments, not more than 3, not more than 2, or not more than 1 dose(s) is/are administered within a particular 12 month period (e.g., within 12 months of the initial dose).

In some embodiments, evaluation of an induced immune response (e.g., of magnitude, character, and/or diversity of immune response, such as antibody and/or T cell response) is performed before and/or after one or more doses (e.g., 1, 2, 3, 4, or more weeks after administration of a particular dose and/or within 6, 5, 4, 3, 2, or 1 month of administration of a particular dose) and may, for example be considered in determination of whether one or more booster doses should be administered and/or timing of such booster dose administration. In some embodiments, assessment of an immune response may utilize, for example, techniques that determine presence and/or level of epitope-specific CTL populations in a PBMC sample.

### Example 9. Administration of Dendritic Cells

The present Example describes exemplary dendritic cell compositions that comprise or deliver antigens as described herein.

In this example, peptides comprising epitopes as described herein (e.g., identified, designed, selected and/or characterized as described herein) are loaded onto dendritic cells. Peptide-pulsed dendritic cells can be administered to a subject. In some embodiments, such administration may stimulate a CTL response in vivo.

In this particular Example, dendritic cells (e.g., autologous dendritic cells) are isolated, expanded, and pulsed with peptide CTL and/or HTL epitopes as described herein. Dendritic cells may then be infused back into the patient. Such infusion can elicit CTL and/or HTL responses in vivo. The induced CTL and HTL then destroy (CTL) or facilitate destruction (HTL) of target cells (e.g., liver cells) that bear the proteins from which the epitopes in the vaccine are derived.

Ex vivo CTL or HTL responses to a particular antigen can be induced by incubating in tissue culture the patient's, or genetically compatible, CTL or HTL precursor cells together with a source of antigen-presenting cells, such as dendritic cells, and the appropriate immunogenic peptides.

After an appropriate incubation time (typically about 7-28 days), in which the precursor cells are activated and expanded into effector cells, the cells are infused back into the patient, where they will destroy (CTL) or facilitate destruction (HTL) of their specific target cells, i.e., cells displaying relevant epitopes.

### Example 10: Administration of Epitope Binding Agents

The present Example describes administration of epitope binding agents, as an alternative or complement to vaccination strategies describes herein.

For example, among other things, the present disclosure provides technologies for identification and/or characterization of HSV antigens that are particularly amenable to targeting in order to disrupt one or more features of infection. In many embodiments described herein, the present disclosure provides technologies that involve administration or delivery of antigens that are or comprise such epitopes, for example, in order to induce an immune response targeting such epitope(s) in a recipient.

Alternatively or additionally, the present disclosure provides and encompasses technologies for developing, characterizing, and/or administering agents that bind to such epitopes. In some embodiments, such strategies may provide or represent therapeutic interventions, for example useful in addition or as an alternative to vaccination strategies.

Epitope binding agents, such as antibody agents, TCR agents, CAR agents, and/or cells expressing any of the foregoing can be administered in accordance with methodologies known in the art and/or described herein.

In some embodiments, a relevant epitope binding agent can be delivered by administration of a composition that is or comprises the polypeptide binding agent. Alternatively or additionally, in some embodiments, a relevant epitope binding agent can be delivered by administration of a composition that is or comprises a polynucleotide (e.g., a DNA or RNA and, in many favored embodiments, an RNA) encoding the polypeptide binding agent. In some embodiments, a polypeptide binding agent is delivered by administered by a cell (or population thereof) that comprises or expresses the polypeptide and/or a polynucleotide that encodes it.

### Example 11: Exemplary Identification and/or Characterization of Variant Sequences with Immunogenic Potential

The present Example describes technologies for identification and/or characterization of peptide sequences that differ from a relevant reference, for assessment of immunogenic potential.

The full-length amino acid sequence of a variant protein (e.g., as observed in a circulating strain or developed through a predictive model) was derived.

Constituent 9-mer and 10-mer peptide fragments of the variant protein are each scored for binding potential on common HLA alleles (including, e.g., but not limited to HLA-A01:01, HLA-A02:01. HLA-A03:01, HLA-A24:02, HLA-B07:02, and HLA-B08:01) using available algorithms. Peptides scoring better than 1000 nM are noted as potential candidates.

Alternatively or additionally, constituent 9-mer or 10-mer peptide sequences not found in the reference protein sequence are flagged and scored for binding potential on common HLA alleles (including, e.g., but not limited to HLA-A01:01, HLA-A02:01. HLA-A03:01, HLA-A24:02, HLA-B07:02, and HLA-B08:01) using available algorithms.

### Example 12: Exemplary HSV Peptide String Construct Designs

The present Example exemplifies certain constructs (referred to herein as “strings”) of multiple HSV antigens and/or epitopes present in, and/or linked to one another in, vaccine compositions or otherwise as described herein.

Strings described in the present Example are designed to contain specific epitopes of HSV, each of which is disclosed herein and, e.g., is predicted and/or selected as described here, for example through use of an MHC-binding algorithm as described herein. The strings presented in the present Example are designed for therapeutic use in preventing and/or treating HSV infection and can be administered as polynucleotide constructs, e.g., mRNA encapsulated in a lipid nanoparticle.

In some embodiments, strings described throughout this disclosure are encoded in an RNA that includes a 5′-UTR and 3′-UTR. Epitopes are interconnected by peptide linkers, encoded by their respective polynucleotide sequences. In some embodiments, one or more linkers may have a specific cleavage site.

### Example 13: Exemplary Antigen Identification, Selection and/or Characterization

The present Example describes identification, selection and/or characterization of certain HSV protein sequences useful as or in (i.e., as part of) antigens as described herein (see Example 1).

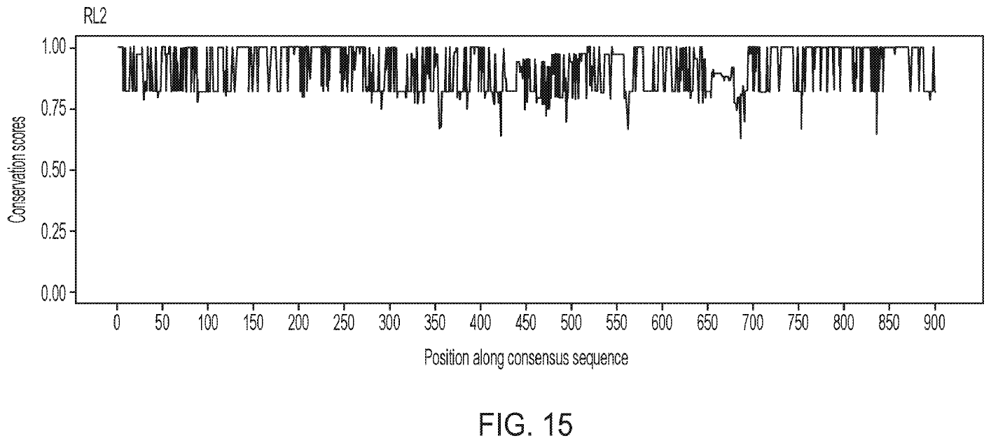

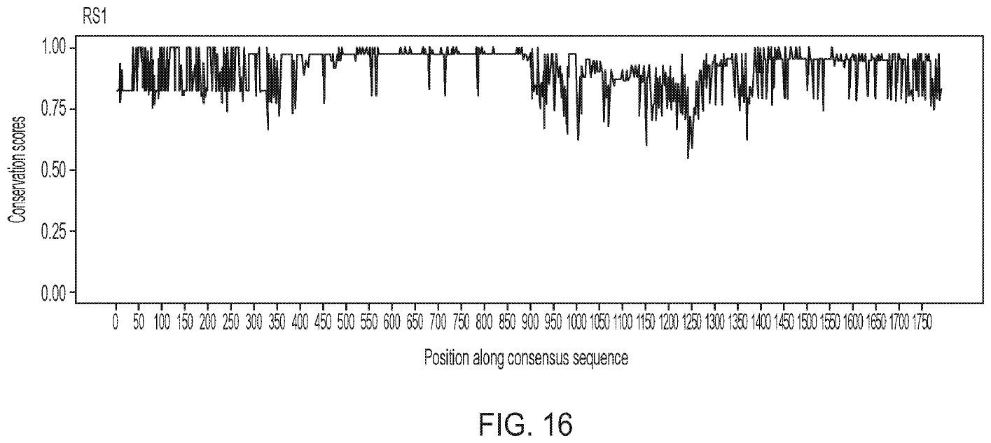

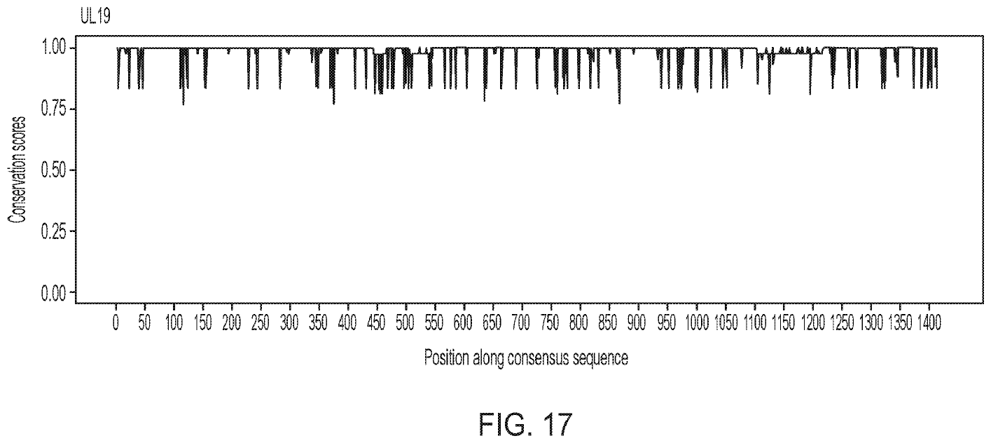

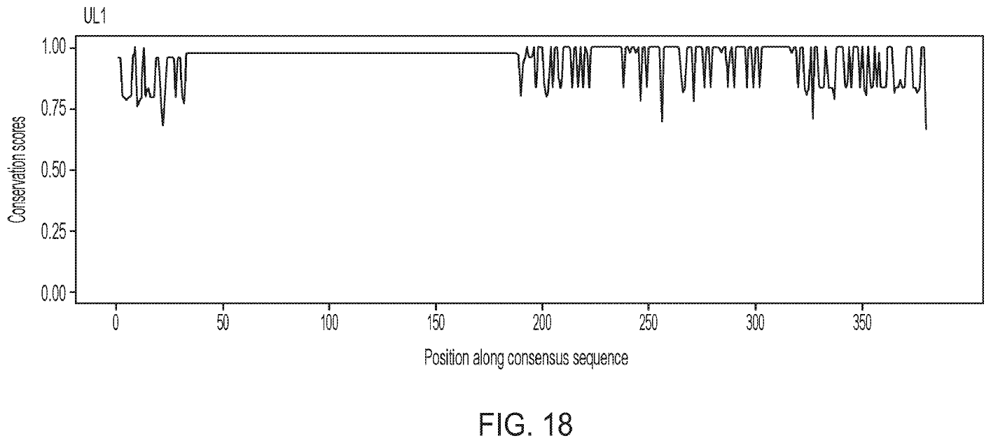

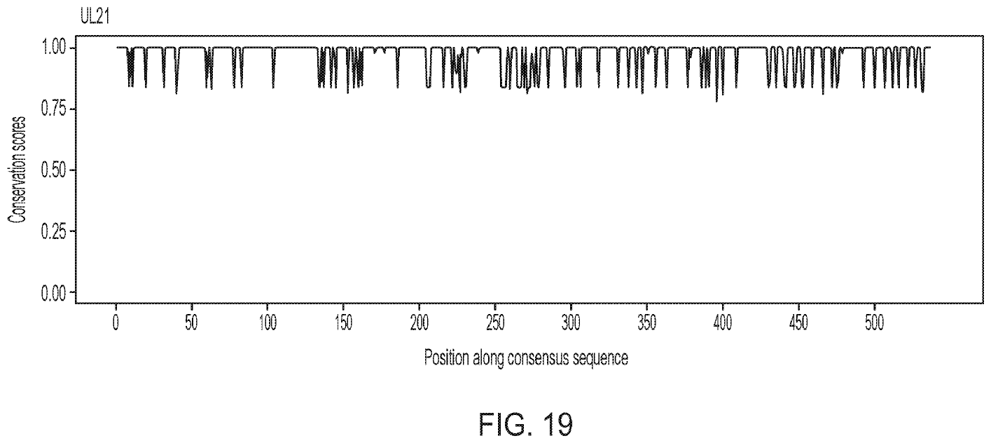

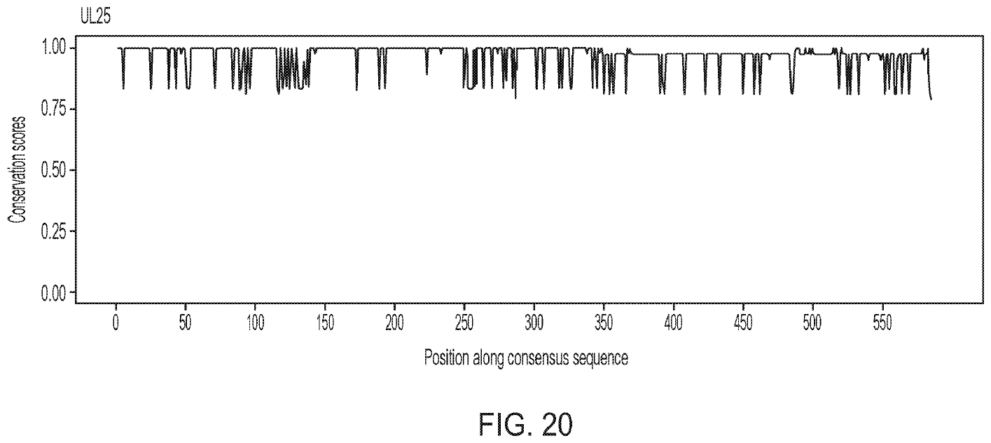

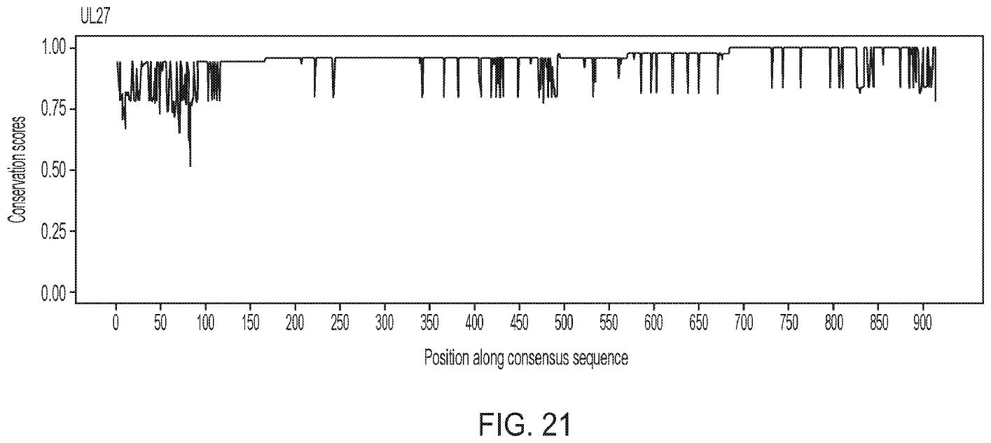

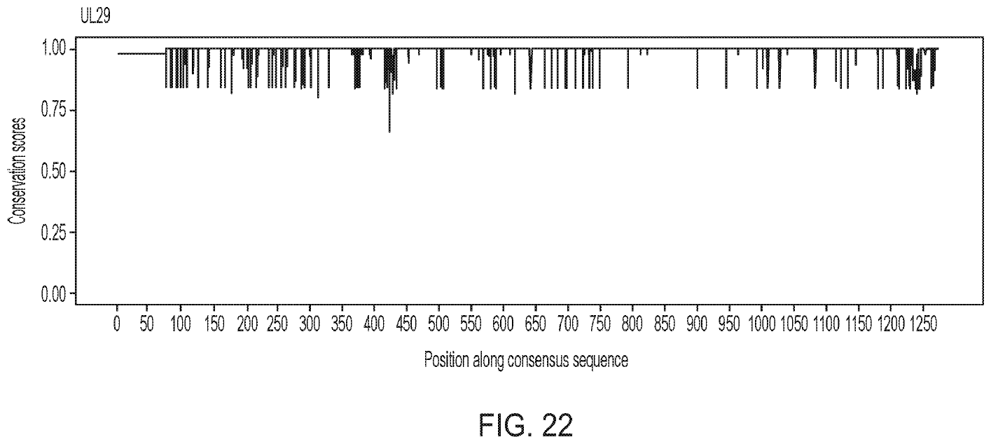

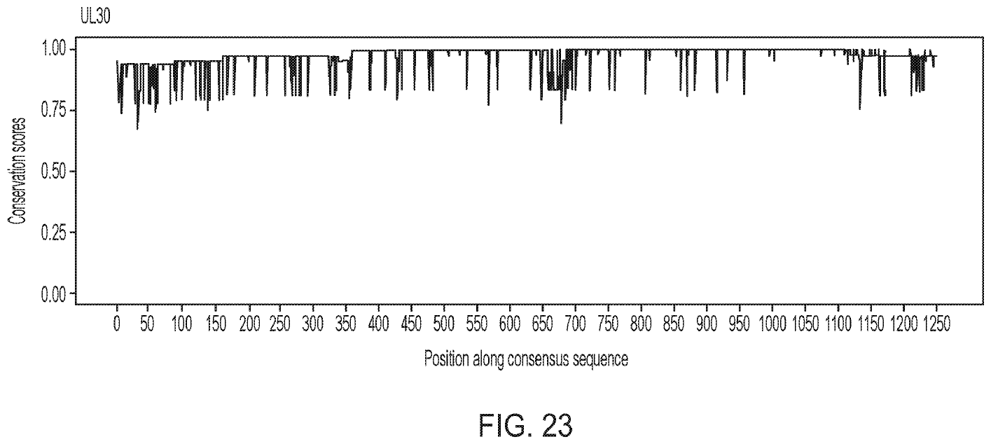

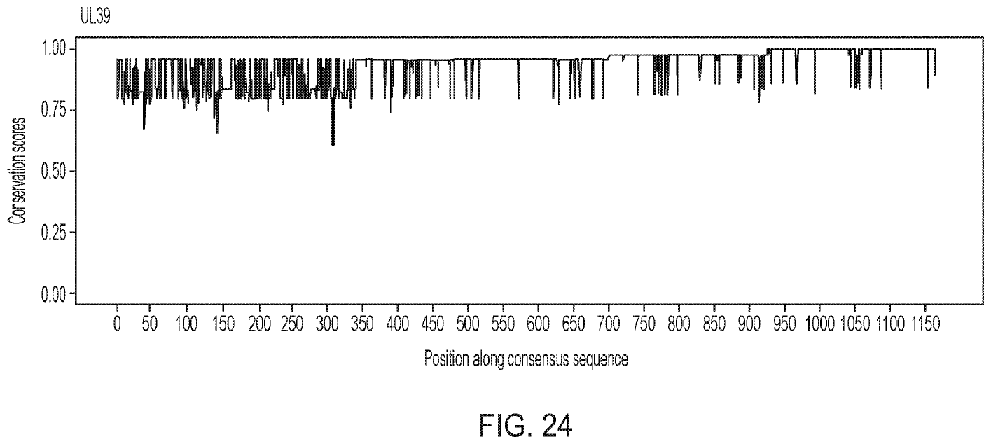

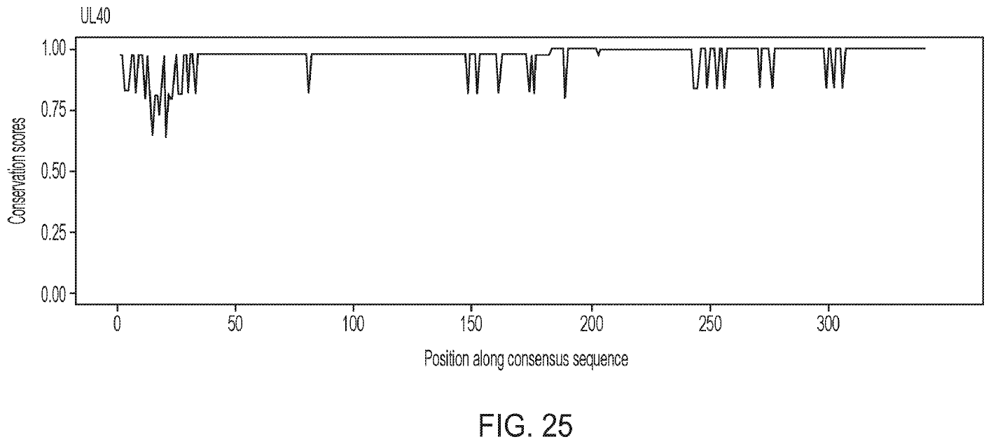

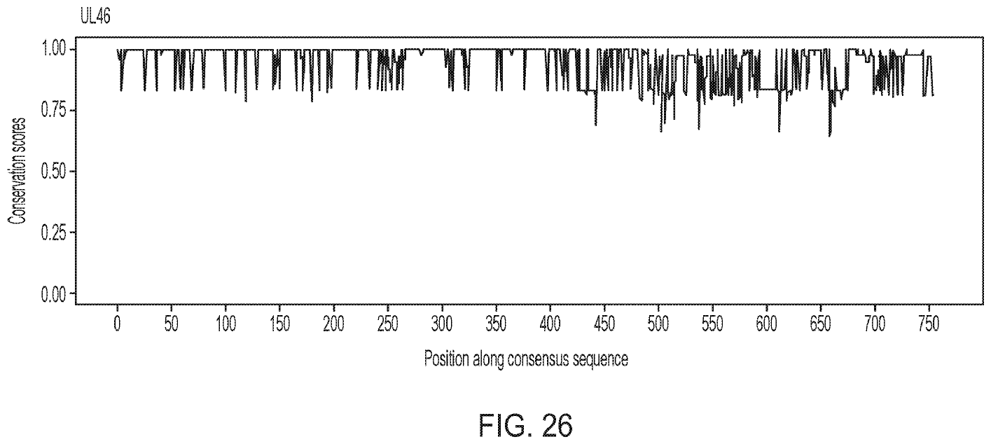

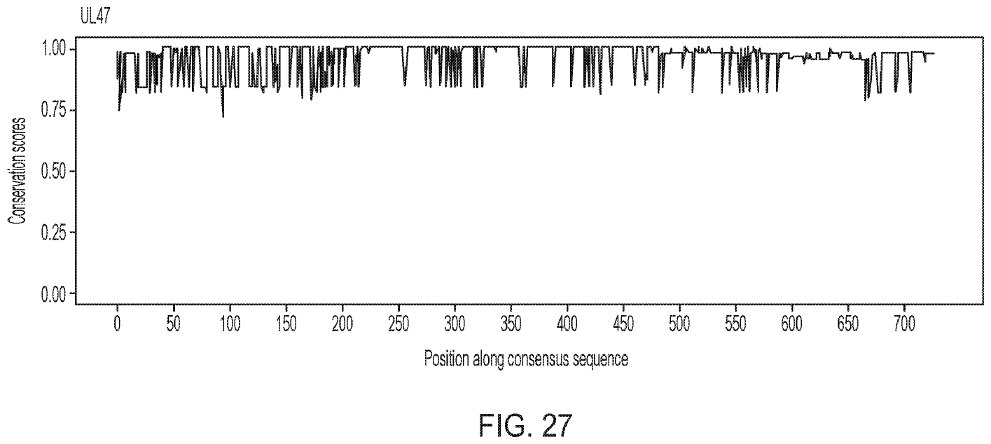

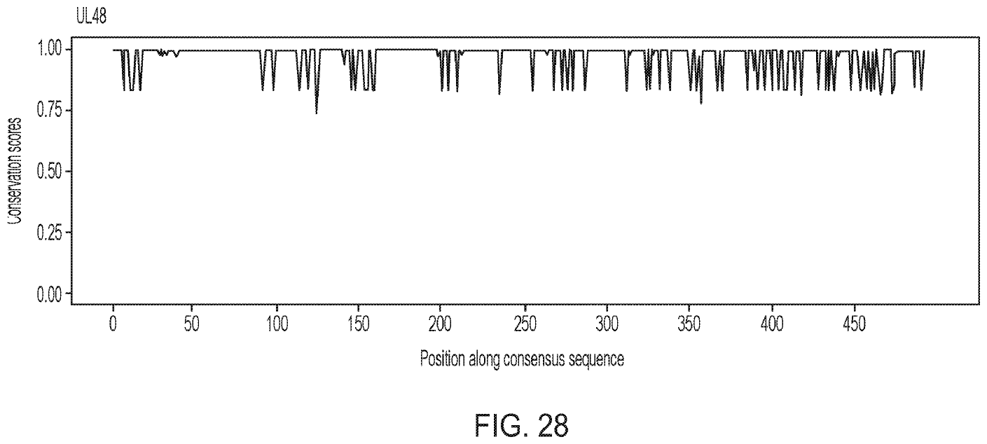

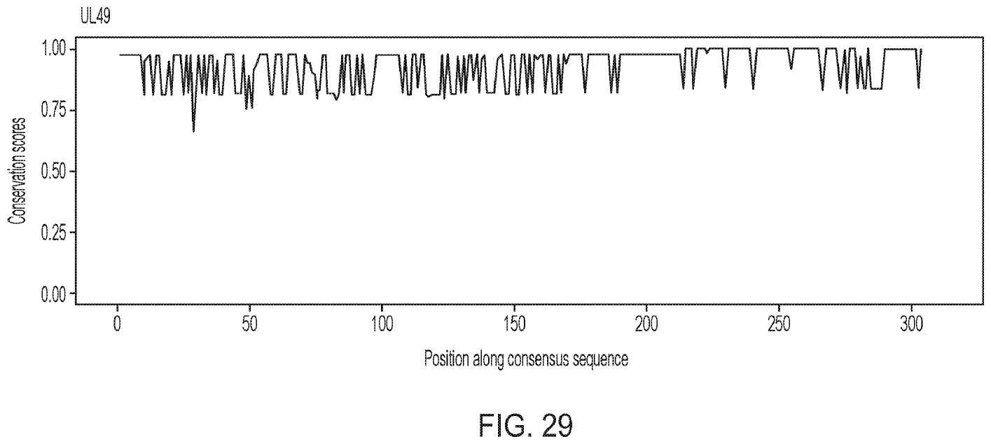

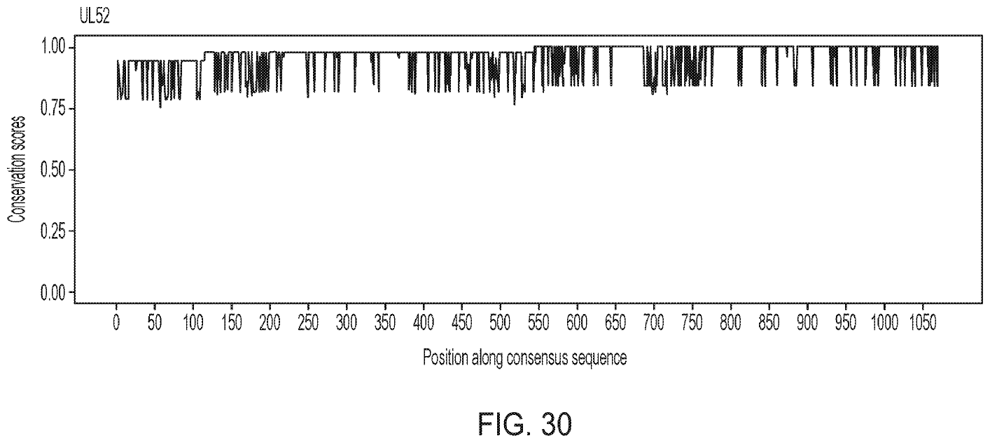

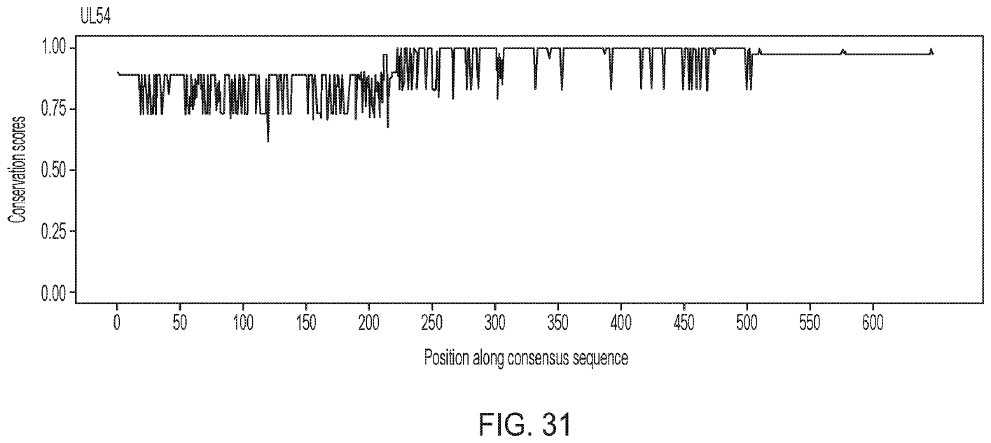

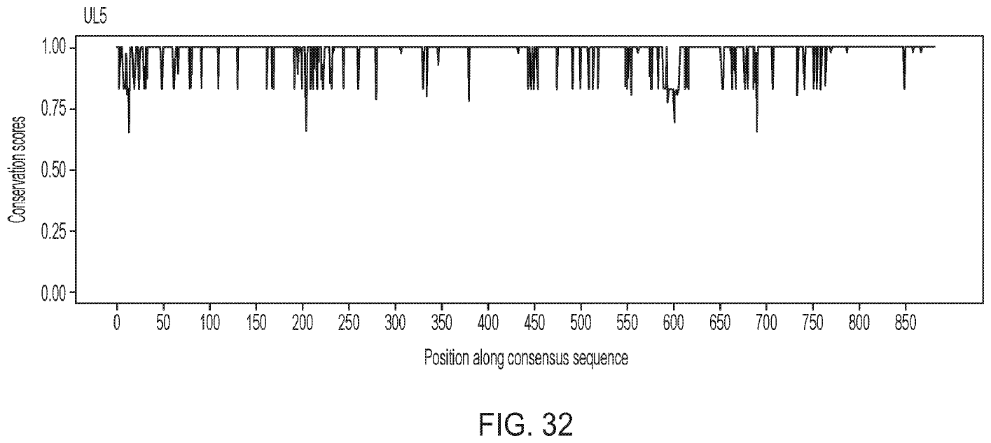

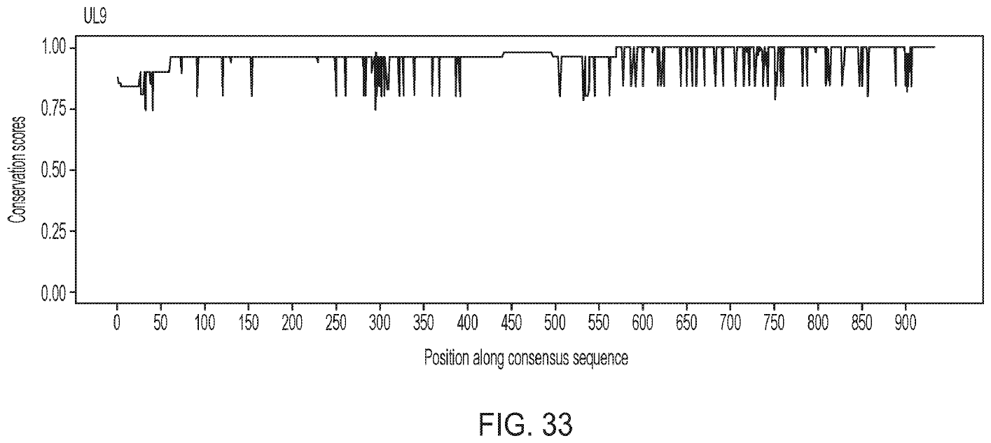

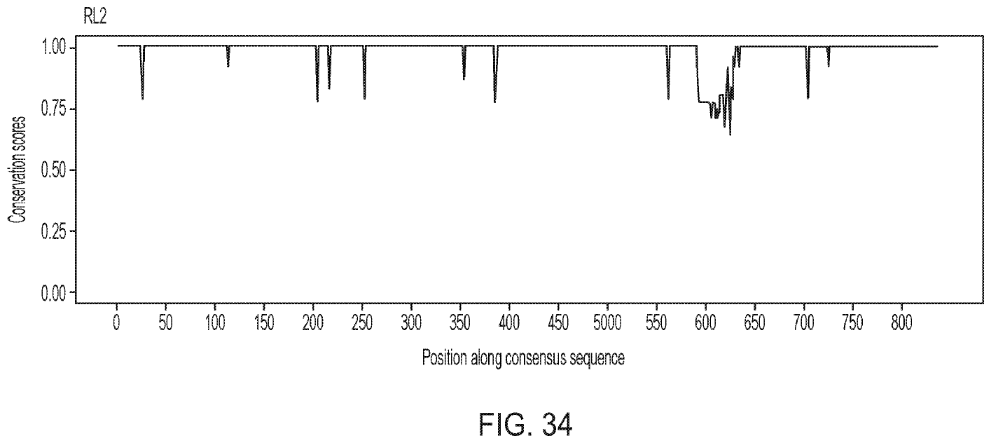

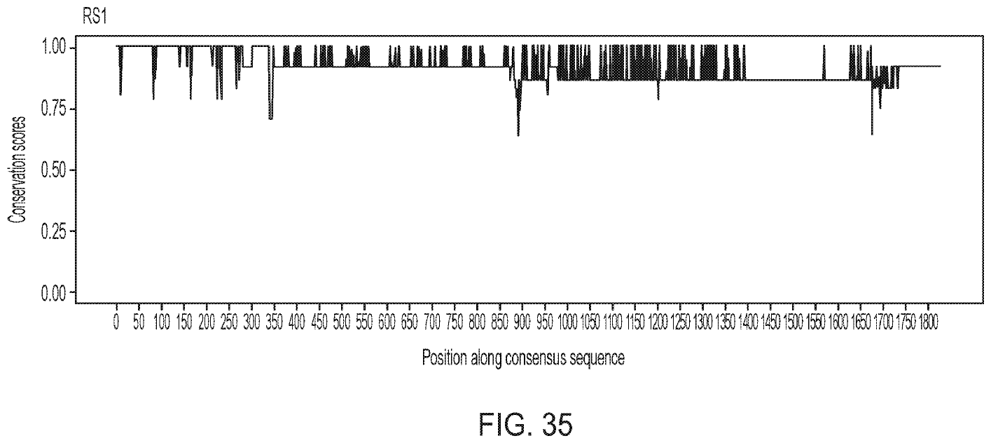

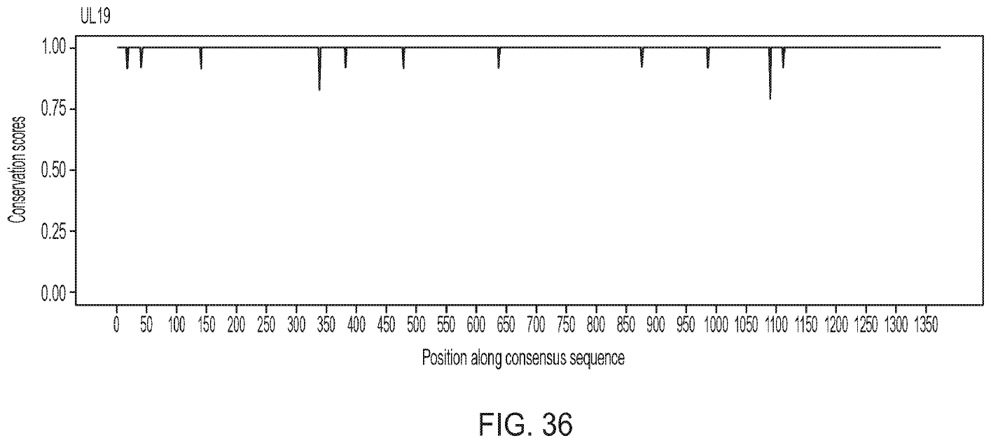

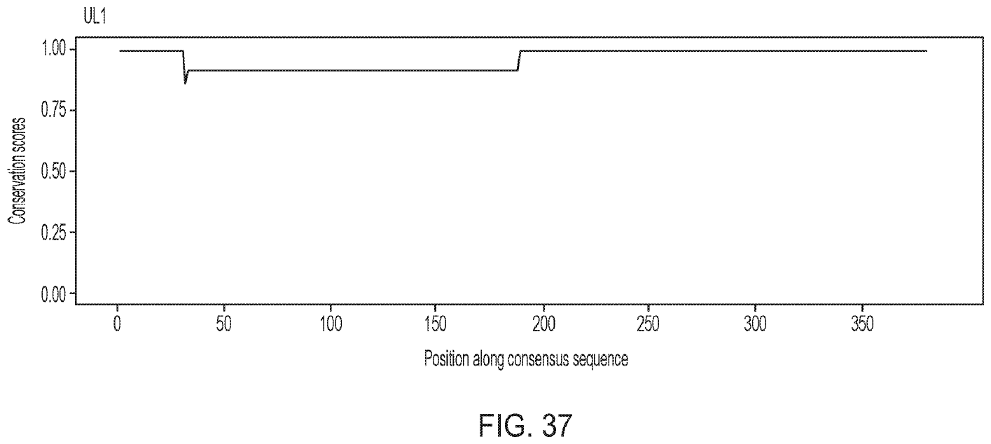

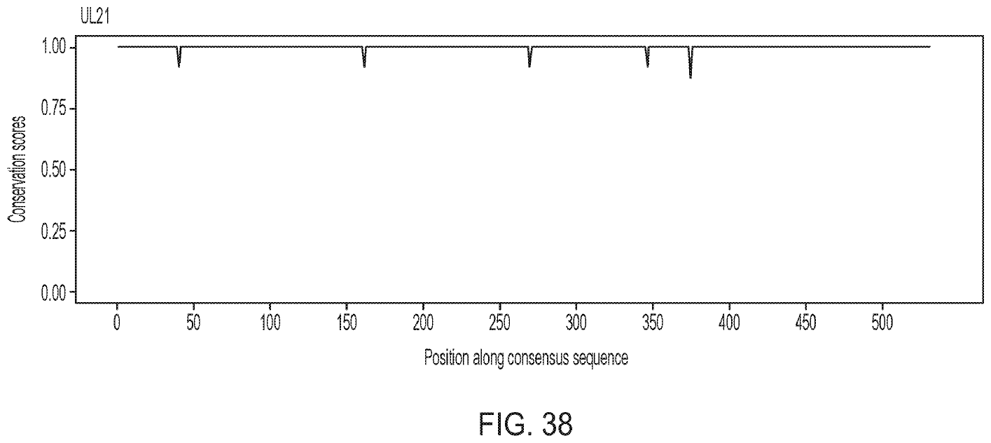

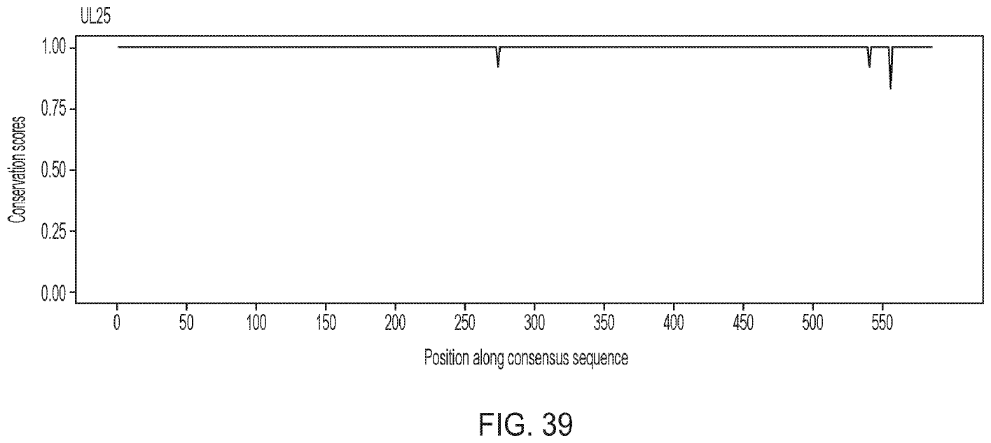

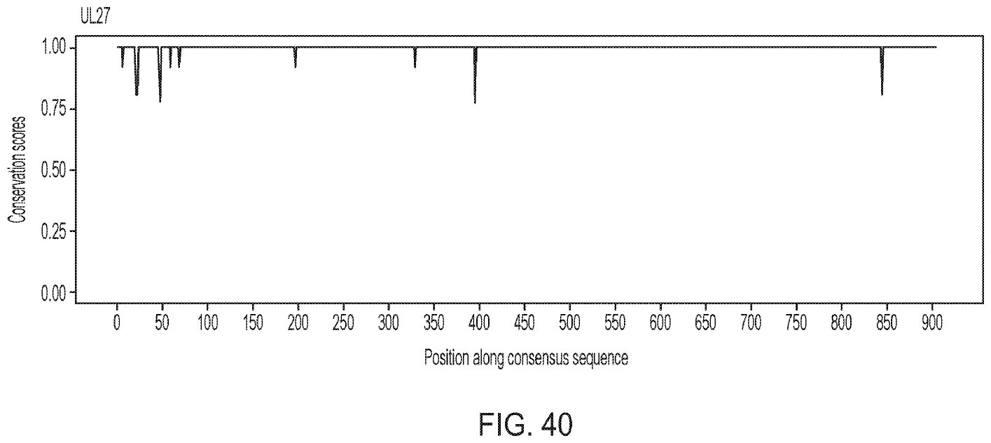

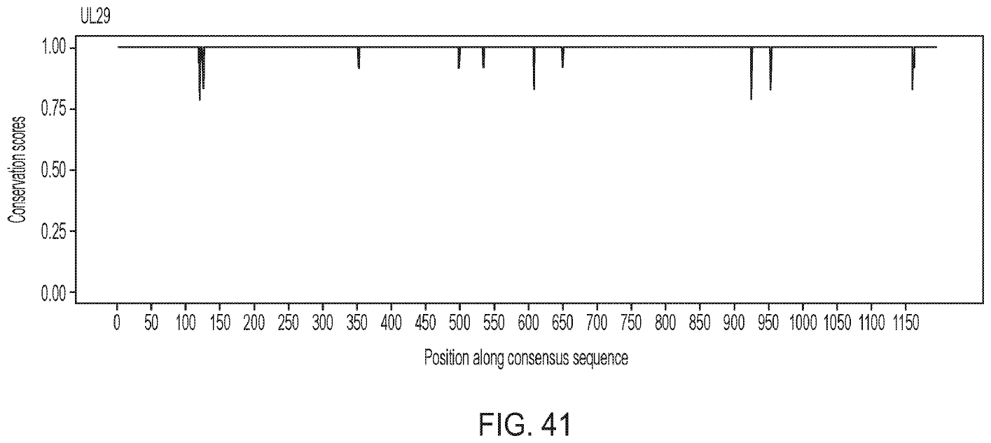

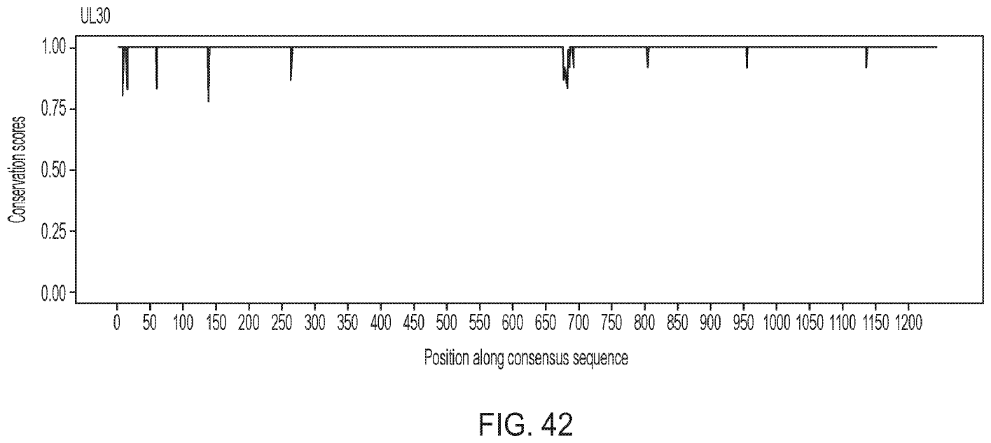

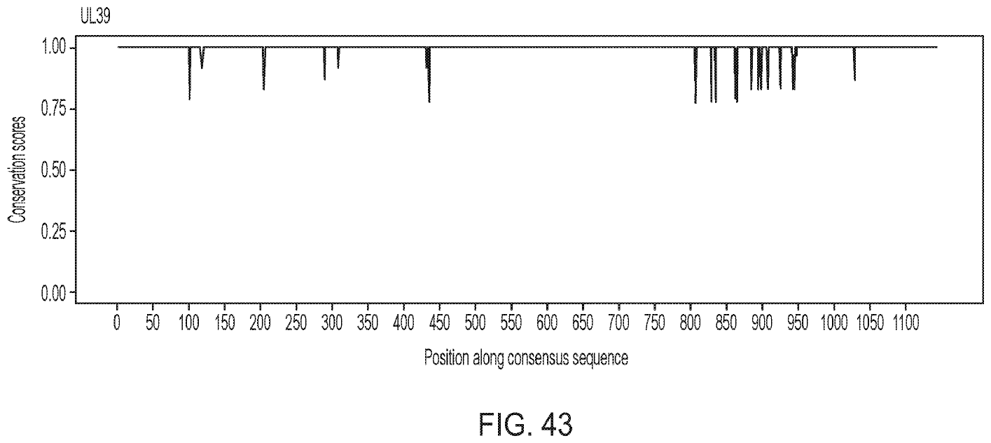

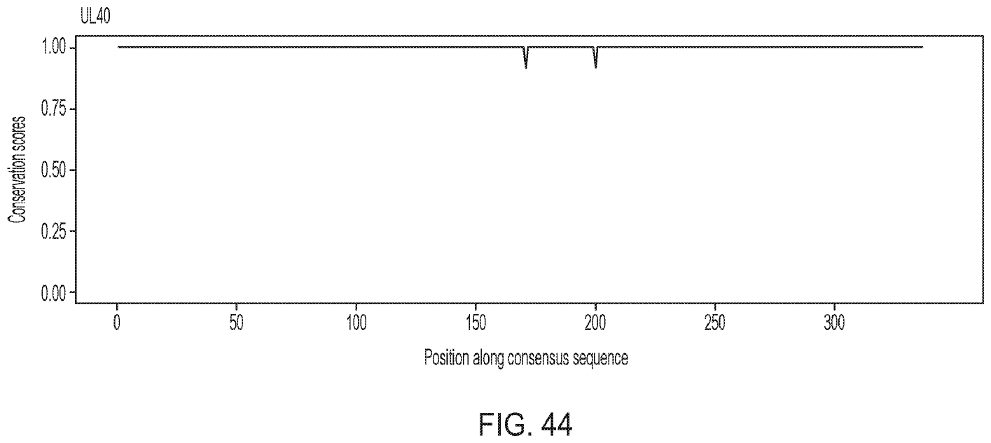

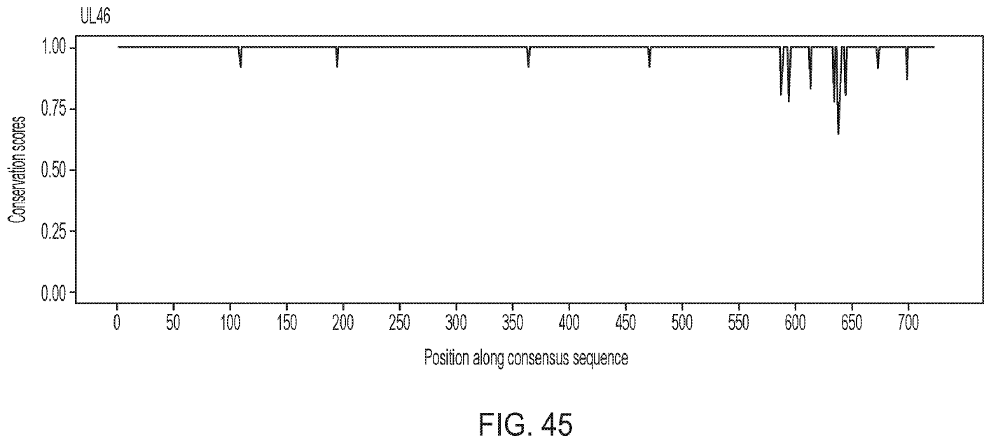

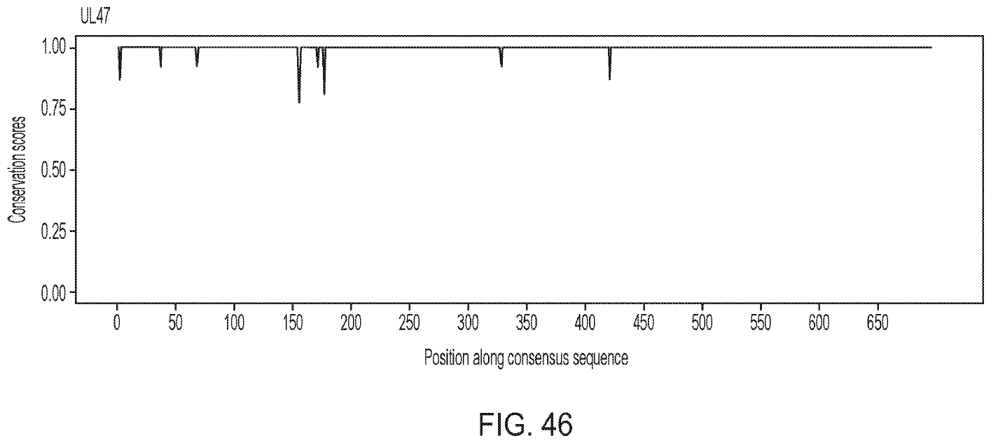

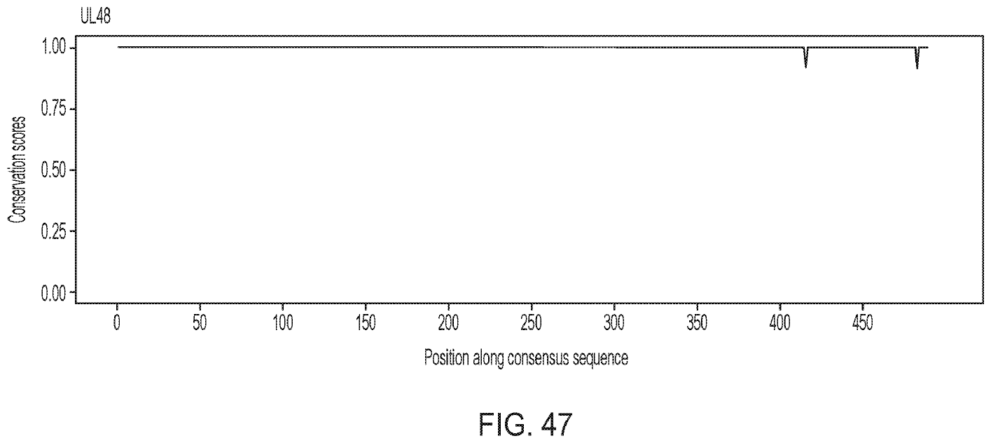

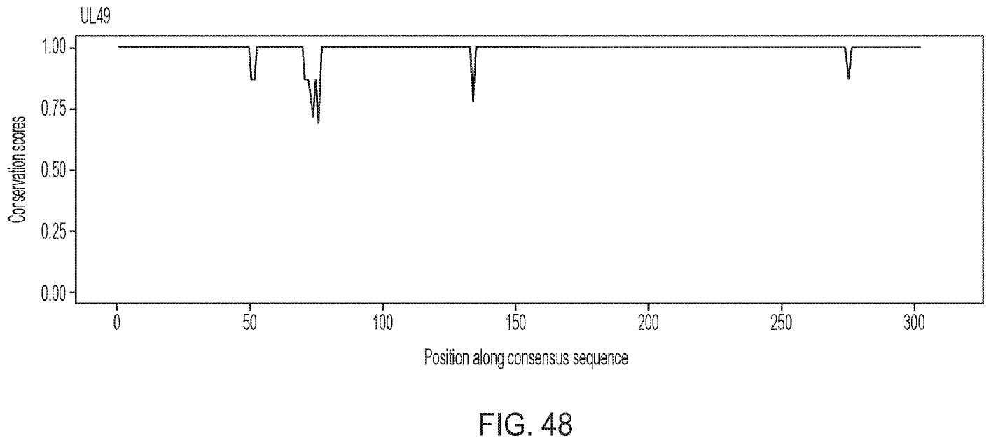

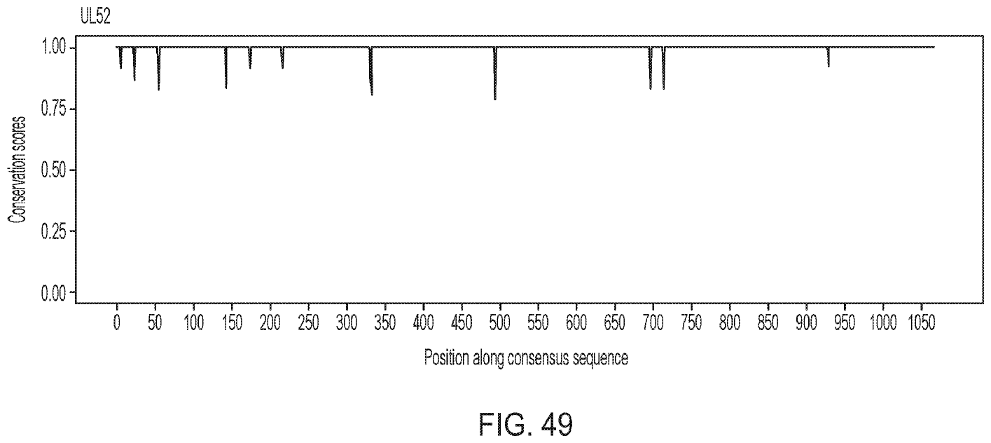

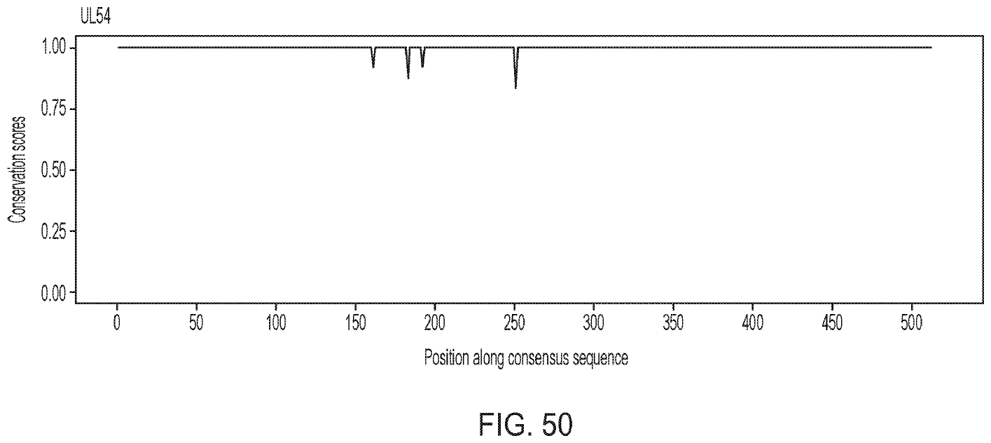

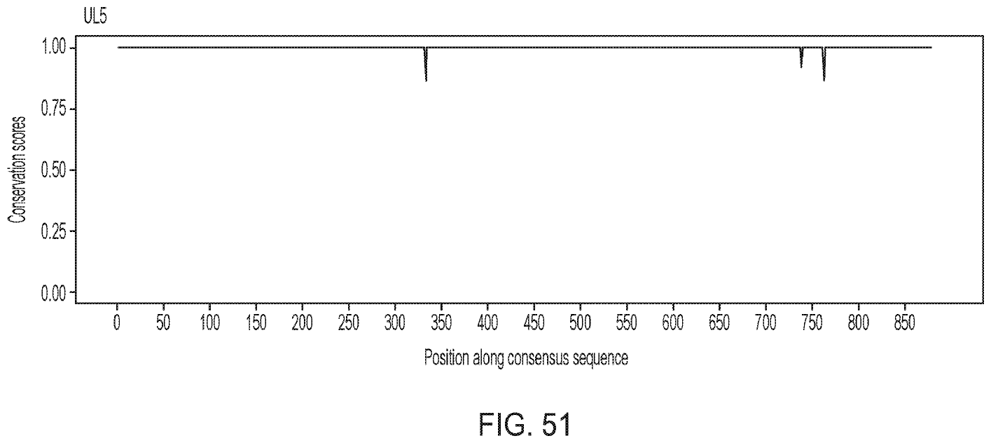

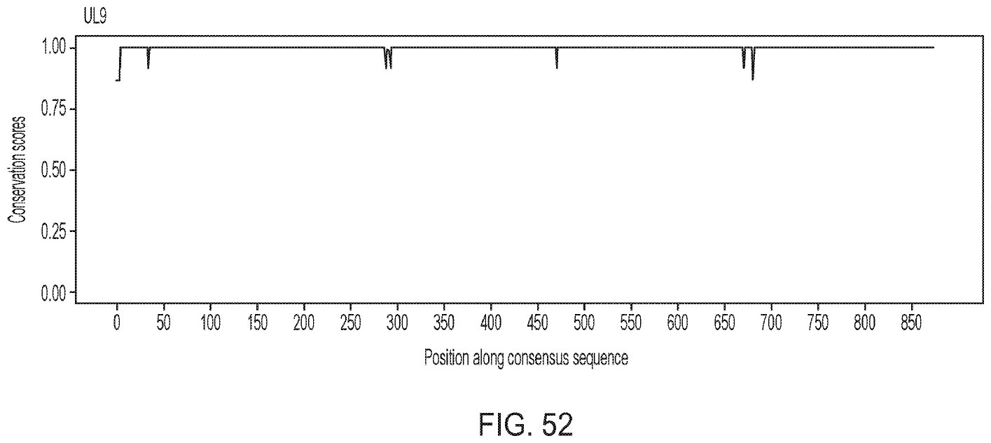

Degree of conservation of candidate proteins across relevant HSV strains (e.g., in relevant geographic region) can be considered, e.g., in selection of sequences and/or epitopes to include in, or encode in, a vaccine and/or antigen construct. Various lab and field isolate strains can be considered for assessing conserved proteins and T cell epitopes. To evaluate conservation of HSV genes identified as a T cell epitopes for HSV, HSV1 and HSV2 genomes were downloaded from VIPR database, using “Genome search->Herpesviridae family->Alphaherpesvirinae subfamily->Simplexvirus Genus->Human Alphaherpesvirus 1 & 2 species”, complete genomes only. All downloaded genomes were checked to have consistent gene names. HSV-1 strain 17 and HSV-2 strain HG52 were used as reference strains for HSV-1 and HSV-2 respectively. The results of this conservation analysis are depicted in FIGS. 15-33. Multiple sequence alignment (MSA) was performed for all sequences belonging to each of the HSV proteins using the mafft-linsi program with default parameters. Two measurements of conservation were computed: 1) protein-level conservation was computed as the percentage of sequence similarity to the reference strains (HSV-1 strain 17 and HSV-2 strain HG52) according to the MSA profiles, and 2) conservation at each position along each protein was quantified as the frequency of the predominant 9-mer that starts at each amino acid position. The results of this HSV strain conservation analysis are depicted in FIGS. 34-52.

Immunogenicity of conserved proteins can also be considered, for example by review of literature and/or application of predictive algorithms as described herein.

In accordance with the present example, in some embodiments, an antigen may be or comprise one or more, and specifically may comprise a plurality, of distinct fragments (e.g., epitope-containing fragments) of one or more of these proteins, for example in a string construct as described herein.

In some embodiments, a secretory signal (“Sec”) or a signal peptide (SP) domain present in exemplary string candidates described herein (e.g., a CD8 T cell antigen string and/or a CD4 T cell antigen string as described herein) may be that from HSV-2 gD SP MGRLTSGVGTAALLVVAVGLRVVCA (SEQ ID NO: 87); in alternative embodiments, a different secretory signal, e.g., from HSV-1 gD SP, is used. In some embodiments, a secretory signal peptide may be or comprise an IgE signal peptide. In some embodiments, a secretory signal peptide may be or comprise an IgE HC (Ig heavy chain epsilon-1) secretory signal peptide. In some embodiments, a secretory signal peptide that may be useful in accordance with the present disclosure may comprise one of the following sequences:

MDSKGSSQKGSRLLLLLVVSNLLLPQGVVG (SEQ ID NO: 118);
MDWTWILFLVAAATRVHS (SEQ ID NO: 93);
METPAQLLFLLLLWLPDTTG (SEQ ID NO: 92);
MLGSNSGQRVVFTILLLLVAPAYS (Japanese encephalitis
PRM signal sequence; SEQ ID NO: 94);
MKCLLYLAFLFIGVNCA (VSVg protein signal sequence;
SEQ ID NO: 95);
MWLVSLAIVTACAGA (Japanese encephalitis JEV signal
sequence; SEQ ID NO: 119);
or
MFVFLVLLPLVSSQC (SEQ ID NO: 120).

In some embodiments, certain chunk boundary considerations are incorporated into the string constructs, for example establishing chunk boundaries to minimize presence of sequences (e.g., epitopes) that may overlap with the human proteome.

### Example 14: Exemplary Vaccine Composition

The present Example describes certain exemplary vaccine compositions:

In some embodiments, a provided vaccine candidate will contain at least 2 RNAs, at least one of which encodes a HSV antigen described herein (e.g., a full length HSV protein or one or more fragments or epitopes thereof, such as a string construct described herein), and optionally at least one of which encodes at least one other conserved HSV protein (or fragment(s) or epitope(s) thereof, such as in a string construct as described herein; in some embodiments such a string construct may include fragments or epitopes from two or more different HSV proteins).

In some embodiments, two or more (e.g., 3, for example 2 of which are/encode HSV antigen string constructs and one of which is/encodes a string construct of a plurality of CD8 and/or CD4 epitopes from other conserved HSV proteins) are formulated together in a single LNP formulation; in other embodiments, individual RNAs may be separately formulated in (the same or different) LNP formulations and such may be mixed together (e.g., in a 1:1 ratio of each RNA, or alternatively in a 1:1 ratio of HSV-antigen-encoding-RNA to “other” RNA so that, for example, for a composition comprising 2 HSV-antigen-encoding RNAs and one other RNA, the ratios would be 0.5:0.5:1).

### Example 15: Exemplary LNP Formulations

The present Example describes certain preferred LNP formulations useful for vaccine compositions as described herein.

In some embodiments, LNP formulations that are useful for vaccine compositions as described herein can comprise at least one ionizable aminolipid. In some embodiments, LNP formulations that are useful for vaccine compositions as described herein can further comprise a helper lipid, which in some embodiments may be or comprise a neutral helper lipid. In some embodiments, LNP formulations that are useful for vaccine compositions as described herein can further comprise a polymer-conjugated lipid, for example in some embodiments PEG-conjugated lipids. In some embodiments, LNP formulations that are useful for vaccine compositions as described herein can comprise at least one ionizable aminolipid, at least one helper lipid (e.g., a neutral helper lipid, which in some embodiments may comprise a phospholipid, a steroid, or combinations thereof), and at least one polymer-conjugated lipid (e.g., PEG-conjugated lipid). In some embodiments, an exemplary LNP formulation may comprise an ionizable aminolipid, a phospholipid, a steroid, and a PEG-conjugated lipid.

In some embodiments, an ionizable aminolipid may be present in an LNP formulation within a range of 45 to 55 mol percent, 40 to 50 mol percent, 41 to 49 mol percent, 41 to 48 mol percent, 42 to 48 mol percent, 43 to 48 mol percent, 44 to 48 mol percent of total lipids. In some embodiments, an exemplary ionizable aminolipid is or comprises ((4-hydroxybutyl)azanediyl)bis(hexane-6,1-diyl)bis(2-hexyldecanoate) (also known as 6-[N-6-(2-hexyldecanoyloxy)hexyl-N-(4-hydroxybutyl)amino]hexyl 2-hexyldecanoate). In some embodiments, an exemplary ionizable aminolipid is or comprises SM-102 (heptadecan-9-yl 8 ((2 hydroxyethyl)(6 oxo 6-(undecyloxy)hexyl)amino)octanoate) or an aminolipid as described in Sabnis et al. “A Novel Amino Lipid Series for mRNA Delivery: Improved Endosomal Escape and Sustained Pharmacology and Safety in Non-human Primates” Mol. Ther. (2018) 26:1509-1519. In some embodiments, an exemplary ionizable aminolipid is or comprises an ionizable aminolipid as disclosed in US2020/0163878 or WO2018/078053, the entire contents of each of which are incorporated herein by reference for the purposes described herein.

In some embodiments, a phospholipid may be present in an LNP formulation within a range of 5 to 15 mol percent, 7 to 13 mol percent, or 9 to 11 mol percent of total lipids. In some embodiments, an exemplary phospholipid is or comprises 1,2-Distearoyl-sn-glycero-3-phosphocholine (DSPC).

In some embodiments, a sterool may be present in an LNP formulation within a range of 30 to 50 mol percent, 35 to 45 mol percent or 38 to 43 mol percent of total lipids. In some embodiments, an exemplary sterol is or comprises cholesterol.

In some embodiments, a polymer conjugated lipid (e.g., PEG-conjugated lipid) may be present in an LNP formulation within a range of 1 to 10 mol percent, 1 to 5 mol percent, or 1 to 2.5 mol percent of total lipids. In some embodiments, an exemplary PEG-conjugated lipid is or comprises 2-[(polyethylene glycol)-2000]-N,N-ditetradecylacetamide (also known as 2-[2-(ω-methoxy (polyethyleneglycol2000) ethoxy]-N,N-ditetradecylacetamide). In some embodiments, an exemplary phospholipid is or comprises PEG2000-DMG (1-monomethoxypolyethyleneglycol-2,3-dimyristylglycerol with polyethylene glycol of average molecular weight 2000). In some embodiments, an exemplary PEG-conjugated lipid is or comprises a PEG-lipid as disclosed in US2020/0163878 or WO2018/078053, the entire contents of each of which are incorporated herein by reference for the purposes described herein.

In some embodiments, an exemplary LNP formulation comprises (i) an ionizable aminolipid within a range of 45 to 55 mol percent of total lipids; (ii) a phospholipid within a range of 8 to 12 mol percent of total lipids; (iii) a steroid within a range of 35 to 45 mol percent of total lipids; and (iv) a polymer conjugated (e.g., PEG-conjugated polymer) within a range of 1 to 2 mol percent of total lipids; and RNA molecules as described herein that are encapsulated within or associated with the lipid nanoparticles.

In some embodiments, an exemplary LNP formulation comprises (i) ionizable amino lipid within a range of 45 to 55 mol percent of total lipids; (ii) DSPC within a range of 5 to 15 mol percent of total lipids; (iii) cholesterol within a range of 35 to 45 mol percent of total lipids; and (iv) a PEG-conjugated lipid within a range of 1 to 2 mol percent of total lipids; and RNA molecules as described herein that are encapsulated within or associated with the lipid nanoparticles.

In some embodiments, an exemplary LNP formulation comprises (i) an ionizable aminolipid within a range of 40 to 50 mol percent of total lipids; (ii) a phospholipid within a range of 5 to 15 mol percent of total lipids; (iii) a steroid within a range of 35 to 45 mol percent of total lipids; and (iv) a polymer conjugated (e.g., PEG-conjugated polymer) within a range of 1 to 10 mol percent of total lipids; and RNA molecules as described herein that are encapsulated within or associated with the lipid nanoparticles. In some such embodiments, an ionizable aminolipid is or comprises ((4-hydroxybutyl)azanediyl)bis(hexane-6,1-diyl)bis(2-hexyldecanoate) (also known as 6-[N-6-(2-hexyldecanoyloxy)hexyl-N-(4-hydroxybutyl)amino]hexyl 2-hexyldecanoate). In some such embodiments, a phospholipid is or comprises 1,2-Distearoyl-sn-glycero-3-phosphocholine (DSPC). In some such embodiments, a steroid is or comprises cholesterol. In some such embodiments, a polymer conjugated polymer is or comprises 2-[(polyethylene glycol)-2000]-N,N-ditetradecylacetamide (also known as 2-[2-(ω-methoxy (polyethyleneglycol2000) ethoxy]-N,N-ditetradecylacetamide).

In one embodiment, an exemplary LNP formulation comprises the following lipids included in Table 15 below and RNA molecules as described herein.

TABLE 16
Exemplary LNP Formulation

| | Proportion |
|---|---|
| Lipids | (mol %) |
| ((4-hydroxybutyl)azanediyl)bis(hexane-6,1-diyl)bis(2- | 47-49 (e.g., 47.5) |
| hexyldecanoate)(also known as 6-[N-6-(2- | |
| hexyldecanoyloxy)hexyl-N-(4-hydroxybutyl)amino]hexyl | |
| 2-hexyldecanoate) | |
| 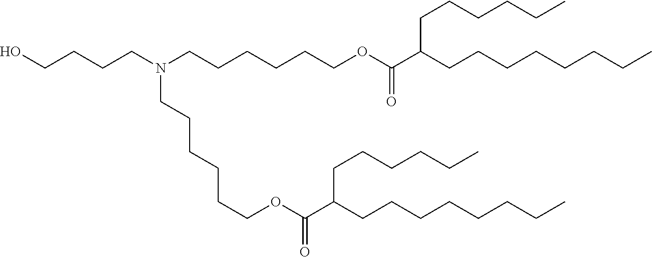 [SMILES: CCCCCCCCC(CCCCCC)C(=O)OCCCCCCN(CCCCO)CCCCCCOC(=O)C(CCCCCC)CCCCCCCC] | |
| 2-[(polyethylene glycol)-2000]-N,N-ditetradecylacetamide | 1.5-2.0 (e.g., 1.8) |
| (also known as 2-[2-(ω-methoxy(polyethyleneglycol2000) | |
| ethoxy]-N,N-ditetradecylacetamide) | |
| 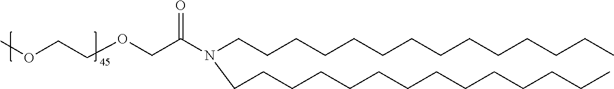 | |
| DSPC | 9.5-10.5 (e.g., 10) |
| Cholesterol, synthetic | 39-42 (e.g., 40.7) |

In some embodiments, an exemplary LNP formulation comprises an ionizable aminolipid, DSPC, cholesterol, and PEG-conjugated lipid at a molar ratio of approximately 50:10:38.5:1.5 or 47.5:10:40.8:1.7. In some embodiments, an ionizable amino lipid is or comprises ((4-hydroxybutyl)azanediyl)bis(hexane-6,1-diyl)bis(2-hexyldecanoate) (also known as 6-[N-6-(2-hexyldecanoyloxy)hexyl-N-(4-hydroxybutyl)amino]hexyl 2-hexyldecanoate).

In some embodiments, an exemplary LNP formulation comprises (i) SM-102 (heptadecan-9-yl 8 ((2 hydroxyethyl)(6 oxo 6-(undecyloxy)hexyl)amino)octanoate) within a range of 45 to 55 mol percent of total lipids; (ii) DSPC within a range of 5 to 15 mol percent of total lipids; (iii) cholesterol within a range of 35 to 45 mol percent of total lipids; and (iv) PEG2000-DMG within a range of 1 to 2 mol percent of total lipids; and RNA molecules as described herein that are encapsulated within or associated with the lipid nanoparticles.

In some embodiments, an exemplary LNP formulation comprises (i) ((4-hydroxybutyl)azanediyl)bis(hexane-6,1-diyl)bis(2-hexyldecanoate) (also known as 6-[N-6-(2-hexyldecanoyloxy)hexyl-N-(4-hydroxybutyl)amino]hexyl 2-hexyldecanoate) within a range of 45 to 55 mol percent of total lipids; (ii) DSPC within a range of 5 to 15 mol percent of total lipids; (iii) cholesterol within a range of 35 to 45 mol percent of total lipids; and (iv) a PEG-conjugated lipid within a range of 1 to 2 mol percent of total lipids; and RNA molecules as described herein that are encapsulated within or associated with the lipid nanoparticles.

### Example 16: Exemplary Pre-Clinical Assessment

The present Example describes certain pre-clinical assessments that may be performed of certain RNA vaccine compositions described herein:

In some embodiments, one vaccine candidate is assessed. In some embodiments, more than one different vaccine candidate may be assessed. In some such embodiments, different candidates may vary, for example, in:
RNA platform (e.g., unmodified RNA, modified RNA, saRNA);
Encoded antigen(s);
Number of RNAs;
Elements of RNA construct (e.g., cap and/or cap-adjacent sequences, 5′-UTR, 3′-UTR, and/or PolyA tail); and/or
Lipid composition of LNP.

In some embodiments, pre-clinical assessment of certain RNA vaccine compositions (e.g., LNP formulated mRNA-based HSV vaccines) comprises one or more of assessment in challenge experiments, assessment of level of protection, assessment of immunogenicity, and/or assessment of functional antibody responses.

LNP formulated mRNA-based HSV vaccines are tested in a challenge model. Non-human primate models, such as Rhesus macaques and Cynomolgus monkey, and/or rodent models, such as C57/B16 mice, Balb/c mice or NODscidIL2Rγnull mice; and/or guinea pig models, inoculated with HSV, are administered a first vaccination and can be administered an additional vaccination (e.g., 1, 2, 3, 4, 5, 6, 7, 8, 9, or 10 additional vaccinations) following the first vaccination. Wherein more than one vaccination is administered, the vaccinations are administered at an interval of 1, 2, 3, 4, 5, 6, 7, or 8 week intervals). Following vaccination, animals are challenged by HSV. Alternatively or additionally, animals are challenged by intravenous, subcutaneous, and/or intramuscular injection of virus-infected lymphocytes. Lymphocytes can be infected with any suitable strain of HSV. Animals are then evaluated for reduced infection of neurons. In some embodiments, an animal model is challenged in a plurality of instances (e.g., before first vaccination and/or wherein additional vaccinations are administered, at any time point between or after vaccinations). Following challenge, animals subjected to the study may be assessed according to any method known in the art, including, for example, serology assessment, immunogenicity, level of protection, etc.

In some embodiments, serum antibody characterization and/or serum transfer experiments (e.g., from one vaccinated species to a different non-vaccinated species, e.g., from vaccinated non-human primate to non-vaccinated mouse) are conducted (e.g., to assess protective antibody response).

In some embodiments, certain RNA vaccine compositions of the present disclosure are assessed for level of protection. Level of protection can be assessed according to any suitable method known in the art.

In some embodiments, certain RNA vaccine compositions of the present disclosure are assessed for immunogenicity. For example, ELISA can be used to determine IgG specific (and subclasses thereof) titers and/or avidity of antibodies generated in response to certain RNA vaccine compositions of the present disclosure to HSV antigens. In some embodiments, serum antibody titers against HSV glycoprotein (e.g., gH and/or gL glycoprotein, etc.) is determined by ELISA using standard methods. In some embodiments, for example, ELISpot (e.g., for CD8+, CD4+ T cells and/or IFNγ) and assessment of pro-inflammatory cytokine responses with splenocytes from immunized and/or challenged animal models and peptide pools derived from vaccine targets can also be assessed. In some embodiments, for example, phenotyping of immune responses (e.g., by flow cytometry) are assessed. In some embodiments, for example, T cell depletion and/or protection assays are conducted to assess immunogenicity (e.g., according to any suitable known method in the art).

In some embodiments, one or more functional responses of antibodies generated in response to certain RNA vaccine compositions of the present disclosure are assessed. Functional antibody responses can be assessed, for example, using a HSV neutralization assay. In some embodiments, a HSV in vitro neutralization assay is performed to evaluate one or more anti-HSV glycoprotein (e.g., HSV gB, gD gH, gL) antibodies in neutralizing HSV. For example, anti-HSV glycoprotein antibodies are obtained by collecting the sera of animals (e.g., mice) vaccinated with HSV mRNA vaccines. HSV virus are added to the diluted sera and neutralization is allowed to continue for 1 hour at room temperature. 3T3 cells are seeded in 96-wells one day before and the virus/serum mixtures are added to 3T3 monolayers. The cells are fixed on the next day and HSV-specific staining is performed. The plates are scanned and analyzed. A neutralization titer is expressed as the highest serum dilution required to achieve a 50% reduction in the number of plaques.

In some embodiments, functional antibody responses can be assessed, for example, using passive transfer studies of sera from immunized animals to naïve animals that are challenged and assessing level of protection.

### Example 17: Exemplary Characterization Studies

The present Example describes certain potential characterization studies that may be utilized, for example, to identify, select, and/or characterize vaccine candidates or vaccine compositions (e.g., manufacturing batches thereof), or components thereof as described herein.

A immunization protocol can be utilized to assess ability of a vaccine candidate that comprises or delivers an antigen(s) as described herein to induce B- and/or T-cells, e.g., after intramuscular immunization, directed to the antigen(s) and/or epitope(s) thereof. In some embodiments, level and/or diversity of response is determined. In some embodiments, presence and/or level of neutralizing antibodies is/are determined. In some embodiments, protection of the immunized subject from challenge with HSV is assessed.

Alternatively or additionally, in some embodiments, one or more in vitro assessments may be performed, for example:
(1) in vitro expression of an antigen encoded by an RNA included in a vaccine composition; and/or
(2) in vitro potency of antigen expressed from an RNA included in a vaccine composition as described herein.

### Example 18: Exemplary Clinical Studies of RNA Vaccine Compositions

The present Example describes certain clinical assessments that may be performed of certain RNA vaccine compositions described herein.

In some embodiments, more than one different vaccine candidate may be assessed. In some such embodiments, different candidates may vary, for example in:
(1) RNA platform (e.g., unmodified RNA, nucleoside-modified RNA, self-amplifying RNA (saRNA), trans-amplifying RNA);
(2) encoded antigen—e.g.,
   which HSV (HSV-1 and/or HSV-2) protein(s) utilized
   full length protein antigen vs fragment vs plurality of fragments vs fusion with one or more heterologous sequences (e.g., membrane tether, secretion, linker(s))
   epitopes from different (and/or multiple) phases of HSV life cycle
(3) number of RNAs
(4) elements of RNA construct
   cap and/or cap-adjacent sequences
   5′ UTR
   3′ UTR
   polyA tail
(5) lipid composition of LNP.

In one particular embodiment, up to three candidate vaccines that have only 1 mRNA encoding for a HSV glycoprotein (e.g., HSV gB, gD gH, gL) or a HSV protein or variant are evaluated and/or up to three candidates that contain 2 mRNAs, one encoding for an HSV glycoprotein (e.g., HSV gB, gD gH, gL) or an HSV protein or variants and another one encoding for CD8 and/or CD4 epitopes from conserved antigens (and optionally considering conserved T-cell epitopes from various stages of the HSV life cycle are evaluated. In this particular exemplary embodiment, vaccine candidates may be evaluated by intramuscular administration, for example, based on a dose-escalation scheme.

### Example 19: Exemplary Production, Characterization, and or Use of Certain Polyepitopic Vaccine Compositions

In some embodiments, immunogenicity of a multi-epitopic RNA or polypeptide is tested in rodents (e.g., mice, e.g., transgenic mice) and/or larger animals such as non-human primates to evaluate the magnitude of immune response induced against the epitopes tested. In some embodiments, immunogenicity of encoded epitopes in vivo can be correlated with in vitro responses of specific CTL lines against target cells expressing the multi-epitope polypeptides. Thus, in some embodiments, such exemplary experiments can show that a multi-epitopic construct serves to both: 1) generate a cell mediated and/or humoral response and 2) that the induced immune cells recognized cells expressing the encoded epitopes.

In some embodiments, for example, to create a DNA sequence encoding the selected multi-epitope construct (e.g., DNA or RNA) for expression in human cells, amino acid sequences of epitopes to be included can be reverse translated. A human codon usage table can be used to guide the codon choice for each amino acid.

In some embodiments, epitope-encoding DNA sequences are directly adjoined, so that when transcribed and translated, a continuous polypeptide sequence is created.

In some embodiments, expression and/or immunogenicity is optimized. In some such embodiments, expression and/or immunogenicity is optimized by incorporating additional elements into an encoding construct. Without limitation, examples of amino acid sequences that can be reverse translated and included in a multi-epitopic construct sequence include, for example: HLA class I epitopes, HLA class II epitopes, an ubiquitination signal sequence, and/or an endoplasmic reticulum targeting signal. In some embodiments, HLA presentation of CTL and HTL epitopes can be improved by including synthetic (e.g., poly-alanine) or naturally-occurring flanking sequences adjacent to the CTL or HTL epitopes; larger peptides comprising the epitope(s) are within the scope of the present disclosure.

In some embodiments, a multi-epitope-encoding DNA sequence can be produced by assembling oligonucleotides that encode the plus and minus strands of the construct. In some embodiments, overlapping oligonucleotides (e.g., 30-100 bases long) can be synthesized, phosphorylated, purified and annealed under appropriate conditions using suitable technique known in the art. In some embodiments, ends of utilized oligonucleotides can be joined, for example, using ligation (e.g., T4 DNA ligation). In some embodiments, synthetic constructs encoding a multi-epitopic can then be cloned into a desired expression vector (e.g., using a suitable cloning technique known in the art).

In some embodiments, standard regulatory sequences well known to those of skill in the art (e.g., promoters, enhancers, etc.) can be included to ensure expression of a polypeitopic construct in target cells. In some embodiments, for example, a promoter with a down-stream cloning site for insertion of the polyepitopic construct coding sequence; a polyadenylation signal for efficient transcription termination; an E. coli origin of replication; and an E. coli selectable marker (e.g., ampicillin or kanamycin resistance). In some embodiments, a utilized promoter or promoters is not limited and can be used for this purpose, e.g., the human herpes simplex virus (hHSV) promoter. See, e.g., U.S. Pat. Nos. 5,580,859 and 5,589,466 for other suitable promoter sequences.

In some embodiments, vector modifications are used to optimize expression and/or immunogenicity. In some embodiments, introns are utilized for efficient gene expression, and one or more synthetic or naturally-occurring introns are incorporated into the transcribed region. In some embodiments, inclusion of stabilization sequences (e.g., mRNA stabilization sequences) and/or sequences for replication in mammalian cells are used for increasing expression.

In some embodiments, once an expression vector is selected, a multi-epitopic construct coding sequence is cloned into a polylinker region downstream of a promoter (e.g., generating a “plasmid”). In some embodiments, such a plasmid is transformed into an appropriate E. coli strain, and DNA is prepared using any suitable technique known in the art. In some embodiments, the orientation and DNA sequence of the multi-epitopic encoding sequence, as well as all other elements included in the vector, are confirmed using, for example, restriction mapping and/or DNA sequence analysis. In some embodiments, bacterial cells comprising a desired plasmid can be stored, for example, as a master cell bank and/or a working cell bank.

In some embodiments, immunomodulatory sequences contribute to the immunogenicity, e.g., of nucleic acid vaccine constructs. In some embodiments, such sequences are included in a vector, outside the coding sequence, if desired to enhance immunogenicity. In some embodiments, such sequences are immunostimulatory. In some embodiments, such sequences are ISSs or CpGs.

In some embodiments, a bi-cistronic expression vector which allows production of both multi-epitopic construct and a second protein (e.g., included to enhance or decrease immunogenicity) are used. Without limitation, examples of proteins or polypeptides that can enhance the immune response if co-expressed with a multi-epitopic construct include cytokines (e.g., IL-2, IL-12, GM-CSF), cytokine-inducing molecules (e.g., LeIF), costimulatory molecules, or for HTL responses, pan-DR binding proteins. In some embodiments, helper (HTL) epitopes are fused and/or linked to intracellular targeting signals and expressed separately from expressed CTL epitopes; this allows direction of the HTL epitopes to a cell compartment different than that of the CTL epitopes. If required, this could facilitate more efficient entry of HTL epitopes into the HLA class II pathway, thereby improving HTL induction. In contrast to HTL or CTL induction, specifically decreasing the immune response by co-expression of immunosuppressive molecules (e.g. TGF-β) can be beneficial in certain diseases.

In some embodiments, commercially-relevant quantities of plasmid DNA (e.g., for administration or for production of RNA and/or protein for administration) can be produced, for example, by fermentation in E. coli, followed by purification. In some embodiments, aliquots from a working cell bank are used to inoculate growth medium, and grown to a predetermined level (e.g., saturation) in flasks (e.g., shaker flasks) or a bioreactor according to well-known techniques. In some embodiments, plasmid DNA is purified using standard bioseparation technologies such as, for example, solid phase anion-exchange resins supplied by QIAGEN, Inc. (Valencia, California). In some embodiments, supercoiled DNA is separated from open circular and linear forms using gel electrophoresis or other suitable methods known in the art.

In some embodiments, purified plasmid DNA is prepared for injection into a subject using a variety of formulations. In some embodiments, lyophilized DNA is reconstitution in sterile phosphate-buffer saline (PBS). This approach, known as “naked DNA,” and is currently being used for intramuscular (IM) administration in clinical trials. In some embodiments, to maximize the immunotherapeutic effects of polyepitopic vaccine compositions, an alternative method for formulating nucleic acids (e.g., purified plasmid DNA, in vitro transcribed RNA, etc) can be used. A variety of methods have been described, and new techniques can become available. In some embodiments, cationic lipids are used in the formulation (see, e.g., as described by WO 93/24640; Mannino & Gould-Fogerite, BioTechniques 6(7): 682 (1988); U.S. Pat. No. 5,279,833; WO 91/06309; and Felgner, et al., Proc. Nat'l Acad. Sci. USA 84:7413 (1987). In some embodiments, glycolipids, fusogenic liposomes, peptides and compounds referred to collectively as protective, interactive, non-condensing compounds (PINC) are complexed to purified plasmid DNA to influence variables such as stability, intramuscular dispersion, or trafficking to specific organs or cell types.

In some embodiments, a polynucleotide is introduced into cells by use of high-speed cell deformation. During high-speed deformation, cells are squeezed such that temporary disruptions occur in the cell membrane, thus allowing the nucleic acid to enter the cell. In some embodiments, polypeptides are produced from expression vectors, e.g., in a bacterial expression vector, for example, and the proteins can then be delivered to the cell.

In some embodiments, target cell sensitization is used as a functional assay for expression and HLA class I presentation of encoded CTL epitopes. For example, in some embodiments, a polynucleotide is introduced into a mammalian cell line that is suitable as a target for standard CTL chromium release assays. In some embodiments, a transfection method used is dependent on the final formulation. In some embodiments, electroporation is used, e.g., for “naked” polynucleotides (e.g., DNA). In some embodiments, wherein cationic lipids are utilized, direct in vitro transfection is utilized as a transfection method. In some embodiments, a plasmid expressing marker protein or polypeptide (e.g., green fluorescent protein (GFP)) is co-transfected to allow enrichment of transfected cells (e.g., using fluorescence activated cell sorting (FACS)). In some embodiments, cells are then chromium-51 (⁵¹-Cr) labeled and used as target cells for epitope-specific CTL lines; cytolysis, detected by ⁵¹Cr release, indicates both production of, and HLA presentation of encoded CTL epitopes. In some such embodiments, expression of HTL epitopes can be evaluated in an analogous manner using assays to assess HTL activity.

In some embodiments, in vivo immunogenicity is utilized for functional testing. In some embodiments, rodents (e.g., mice, e.g., transgenic mice expressing appropriate human HLA proteins) are immunized with a polyepitopic vaccine composition (e.g., comprising a DNA or RNA active agent). In some embodiments, dose and route of administration are formulation dependent (e.g., IM for DNA in PBS or LNP-formulated DNA or RNA, intraperitoneal (IP) for lipid-complexed DNA). In some embodiments, for example, twenty-one days after immunization, splenocytes are harvested and restimulated for 1 week in the presence of peptides encoding each epitope being tested. Thereafter, for CTL effector cells, assays are conducted for cytolysis of peptide-loaded, ⁵¹Cr-labeled target cells using standard techniques. Lysis of target cells that are sensitized by HLA loaded with peptide epitopes, corresponding to minigene-encoded epitopes, demonstrates vaccine function for in vivo induction of CTLs. Immunogenicity of HTL epitopes is evaluated in transgenic mice in an analogous manner.

### Example 20: Exemplary Guinea Pig T Cell Assay—Phase 1 Uninfected Guinea Pigs

Step 1. Measure CD4 and CD8 T cells in naïve guinea pigs (n=10).

Naïve guinea pigs will be used to standardize reagents. Two animals will be used per attempt to standardize. PMA will serve as a positive control. Pan-T cell (CD3), CD4, CD8, IFN gamma, TNF alpha, CD45 (pan leukocyte), CD1B3 (B cell marker to exclude B cells) will be standardized.

Step 2. Measure CD4 and CD8 T cells in immunized guinea pigs (n=16)

Three groups of guinea pigs: PBS (n=4), gE2 mRNA-LNP315 (n=4), test ‘T cell string’ according to the present disclosure (n=4) will be tested. Group 1. PBS as control. Group 2. Immunize guinea pigs×2 separated by 4 weeks with gE2 mRNA (15 ug) and measure CD4 and CD8 T cell responses 10-14 days after 2nd immunization (n=4). Group 3. Immunize guinea pigs×2 separated by 4 weeks with test ‘T cell string’ (15 ug) according to the present disclosure and measure CD4 and CD8 T cells 10-14 days after 2nd immunization (n=4). All splenocytes will be harvested at same time. Two extra animals in group 2 will be immunized and 2 extra in group 3 for additional studies if needed (n=4).

### NUMBERED EMBODIMENTS

1. A composition comprising one or more RNA molecules that collectively encode one or more HSV (e.g., HSV-1 and/or HSV-2) antigens or fragments thereof.
2. A composition for delivery of one or more HSV (e.g., HSV-1 and/or HSV-2) antigens or fragments thereof to a subject, optionally wherein the composition comprises one or more RNA molecules that collectively encode the one or more HSV antigens and/or wherein the composition comprises one or more polypeptides comprising the one or more HSV antigens.
3. The composition of item 1 or item 2, wherein the one or more HSV antigens or fragments thereof have at least 80% sequence identity with one or more sequences selected from SEQ ID NOs: 1-74 (Table 1) or a corresponding fragment thereof.
4. The composition of any one of items 1-3, wherein the one or more HSV antigens or fragments thereof have at least 85%, at least 90%, at least 95%, or 100% sequence identity with one or more sequences selected from SEQ ID NOs: 1-74 (Table 1) or a corresponding fragment thereof.
5. The composition of any one of items 1-4, wherein the one or more HSV antigens or fragments thereof have at least 80% sequence identity with one or more sequences selected from SEQ ID NOs: 1-30 and 74 (Table 2) or a corresponding fragment thereof.
6. The composition of any one of items 1-4, wherein the one or more HSV antigens or fragments thereof have at least 85%, at least 90%, at least 95%, or 100% sequence identity with one or more sequences selected from SEQ ID NOs: 1-30 and 74 (Table 2) or a corresponding fragment thereof.
7. The composition of any one of items 1-4, wherein the one or more HSV antigens or fragments thereof have at least 80% sequence identity with one or more sequences selected from SEQ ID NOs: 31-73 (Table 3) or a corresponding fragment thereof.
8. The composition of any one of items 1-4, wherein the one or more HSV antigens or fragments thereof have at least 85%, at least 90%, at least 95%, or 100% sequence identity with one or more sequences selected from SEQ ID NOs: 31-73 (Table 3) or a corresponding fragment thereof.
9. The composition of any one of items 1-8, wherein the one or more RNA molecules comprise a sequence found in Table 11.
10. The composition of any one of items 1-9, wherein at least one of the one or more RNA molecules encodes multiple HSV (e.g., HSV-1 and/or HSV-2) antigens or fragments thereof.
11. The composition of any one of items 1-10, wherein all of the one or more RNA molecules encode multiple HSV (e.g., HSV-1 and/or HSV-2) antigens or fragments thereof.
12. The composition of any one of items 1-10, wherein at least one of the one or more RNA molecules encodes a single HSV (e.g., HSV-1 and/or HSV-2) antigen or fragments thereof.
13. The composition of any one of items 1-12, wherein two or more HSV antigens or fragments thereof are present in, or encoded in one or more of the RNA molecule as, a single polypeptide.
14. The composition of item 13, wherein the single polypeptide further comprises a linker, optionally wherein the linker has a sequence comprising one or more glycine (G) residues and/or one or more serine (S) residues.
15. The composition of item 13 or item 14, wherein the linker is a cleavable linker.
16. The composition of item 13 or item 14, wherein the linker has a sequence found in Table 10 herein.
17. The composition of any one of items 1-16, wherein the one or more RNA molecules comprise sequences encoding one or more HSV antigens that are codon optimized for expression in a subject, optionally wherein the subject is a human.
18. The composition of any one of items 1-17, wherein the one or more RNA molecules comprises a 5′ cap or 5′ cap analog.
19. The composition of item 18, wherein the 5′ cap analog is or comprises Cap0, a Cap1 or a Cap2.
20. The composition of item 18 or item 19, wherein a 5′-cap analog is or comprises m₂^{7,3′-O}Gppp(m₁^{2′-O})ApG.
21. The composition of any one of items 1-20, wherein the one or more RNA molecules comprises a sequence encoding a signal peptide.
22. The composition of item 21, wherein the signal peptide is or comprises a sequence found in Table 7 herein.
23. The composition of item 21, wherein the one or more RNA molecules comprise a sequence encoding a signal peptide found in Table 8 herein.
24. The composition of any one of items 1-23, wherein the one or more RNA molecules comprise at least one non-coding regulatory element.
25. The composition of any one of items 1-24, wherein the one or more RNA molecules comprises a poly-adenine tail.
26. The composition of item 25, wherein the poly-adenine tail is or comprises a modified adenine sequence.
27. The composition of item 25 or item 26, wherein the poly-adenine tail comprises at least 100 A nucleotides.
28. The composition of any one of items 25-27, wherein the poly-adenine tail is an interrupted sequence of A nucleotides.
29. The composition according to item 28, wherein the poly-adenine tail comprises 30 adenine nucleotides followed by 70 adenine nucleotides, wherein the 30 adenine nucleotides and 70 adenine nucleotides are separated by a linker sequence.
30. The composition of any one of items 1-29, wherein the one or more RNA molecules comprises at least one 5′ untranslated region (UTR) and/or at least one 3′ UTR.
31. The composition according to item 30, wherein the at least one 5′-UTR is or comprises a modified human alpha-globin 5′-UTR.
32. The composition according to item 30 or item 31, wherein the at least one 3′-UTR is or comprises a first sequence from the “amino terminal enhancer of split” (AES) messenger RNA and a second sequence from the mitochondrial encoded 12S ribosomal RNA.
33. The composition of any one of items 1-32, wherein the RNA is a modified RNA, which is modified by substitution of some or all uridine residues with a modified uridine residue.
34. The composition of any one of items 1-33, wherein the RNA is modified by N1-methyl-pseudouridine substitution of some or all uridine residues.
35. The composition of one of items 1-34, wherein the RNA is formulated in lipid nanoparticles comprising a cationic or cationically ionizable lipid, a sterol, a neutral lipid, and a lipid conjugate.
36. The composition of one of items 1-35, wherein the RNA is formulated in lipid nanoparticles comprising a cationically ionizable lipid, a phospholipid, a cholesterol, and a polyethylene glycol (PEG)-lipid.
37. The composition of item 35 or item 36, wherein the cationic lipid or cationically ionizable is or comprises ((4-hydroxybutyl)azanediyl)bis(hexane-6,1-diyl)bis(2-hexyldecanoate), the sterol is or comprises a cholesterol, the neutral lipid is or comprises a phospholipid, and the lipid conjugate is or comprises a polyethylene glycol (PEG)-lipid.
38. The composition according to any one of items 35-37, wherein the one or more lipid nanoparticles comprise:
   a. ((4-hydroxybutyl)azanediyl)bis(hexane-6,1-diyl)bis(2-hexyldecanoate);
   b. a cholesterol;
   c. distearoylphosphatidylcholine (DSPC); and
   d. 2-[(polyethylene glycol)-2000]-N,N-ditetradecylacetamide.
39. The composition of item 35 or 36, wherein the cationically ionizable lipid has the following structure:

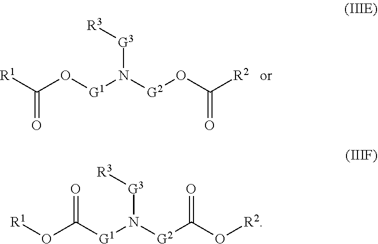

or a pharmaceutically acceptable salt, tautomer or stereoisomer thereof, wherein:
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₅ cycloalkylene, C₃-C₈ cycloalkenylene;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl.
40. The composition of item 39, wherein R¹ or R², or both, has one of the following structures:

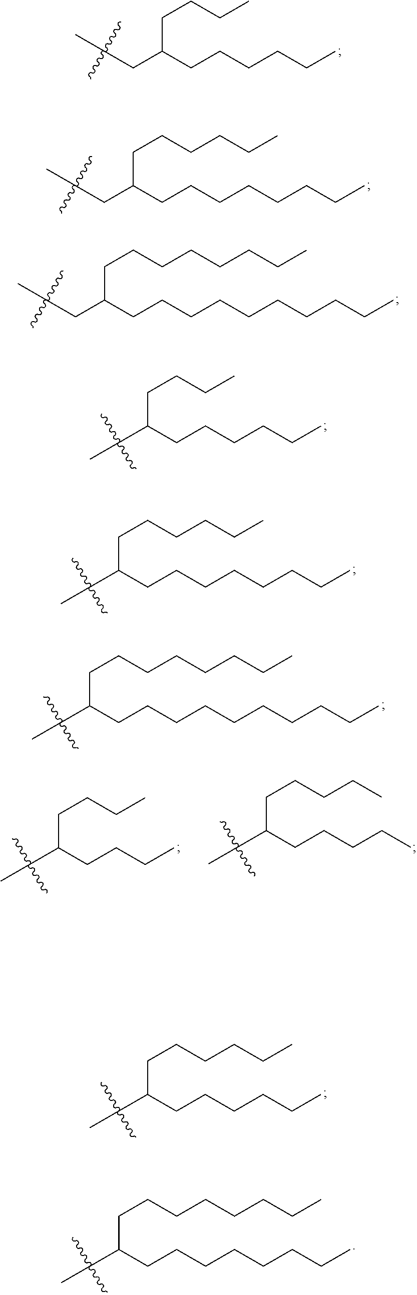

41. The composition of item 39, wherein the cationically ionizable lipid has the following structure:

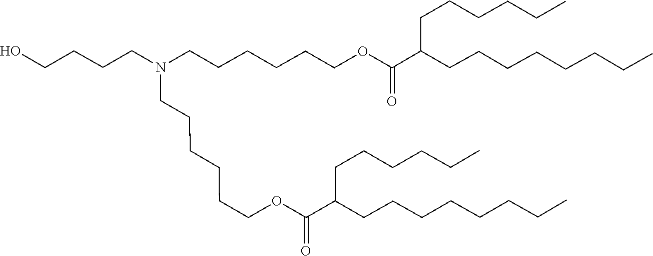 [SMILES: CCCCCCCCC(CCCCCC)C(=O)OCCCCCCN(CCCCO)CCCCCCOC(=O)C(CCCCCC)CCCCCCCC]

42. The composition of any one of items 36-41, wherein the phospholipid is or comprises distearoylphosphatidylcholine (DSPC).
43. The composition any one of items 36-42, wherein the (PEG)-lipid is or comprises 2-[(polyethylene glycol)-2000]-N,N-ditetradecylacetamide.
44. The composition of any one of items 36-43, wherein the phospholipid is present in a concentration ranging from 5 to 15 mol percent of the total lipids.
45. The composition of any one of items 35-44, wherein the cationically ionizable lipid is present in a concentration ranging from 40 to 55 mol percent of the total lipids.
46. The composition of any one of items 36-45, wherein the cholesterol is present in a concentration ranging from 30 to 50 mol percent of the total lipids.
47. The composition of any one of items 36-46, wherein the (PEG)-lipid is present in a concentration ranging from 1 to 10 mol percent of the total lipids.
48. The composition of any one of items 36-47, wherein the lipid nanoparticles comprise from 40 to 55 mol percent of a cationically ionizable lipid; from 5 to 15 mol percent of a phospholipid; from 30 to 50 mol percent of a cholesterol; and from 1 to 10 mol percent of a PEG-lipid.
49. The composition of item 38, wherein ((4-hydroxybutyl)azanediyl)bis(hexane-6,1-diyl)bis(2-hexyldecanoate) is within a range of about 40 to about 55 mole percent, a cholesterol is within a range of about 30 to about 50 mole percent, distearoylphosphatidylcholine (DSPC) is within a range of about 5 to about 15 mole percent, and 2-[(polyethylene glycol)-2000]-N,N-ditetradecylacetamide is within a range of about 1 to about 10 mole percent.
50. The composition of any one of items 1-49, further comprising at least one salt and/or a cryoprotectant, wherein the cryoprotectant is or comprises sucrose.
51. The composition of any one of items 1-50, wherein the total RNA is present in an amount within a range of 1 μg to 100 μg per dose in the composition.
52. A pharmaceutical composition comprising a composition of any one of items 1-51.
53. The pharmaceutical composition of item 52, which is in a liquid formulation.
54. The pharmaceutical composition of item 52, which is in a frozen formulation.
55. The pharmaceutical composition of item 54, wherein the frozen formulation comprises PBS.
56. The pharmaceutical composition of any one of items 52-55, wherein the composition is formulated for intramuscular administration.
57. The pharmaceutical composition of any one of items 52-55, wherein the composition is formulated for intraveneous administration.
58. The pharmaceutical composition of any one of items 52-55, wherein the composition is formulated for subcutaneous administration.
59. A method comprising administering at least one dose of a pharmaceutical composition of any one of items 52-58 to a subject.
60. The method of item 59, wherein the subject is human.
61. The method of item 59 or item 60, wherein the subject is suffering from an HSV (e.g., HSV-1 and/or HSV-2) infection.
62. The method of item 59 or item 60, wherein the subject intends to be present within a geographical region that has a high HSV (e.g., HSV-1 and/or HSV-2) prevalence within the next three months.
63. The method of item 62, wherein a high HSV (e.g., HSV-1 and/or HSV-2) prevalence is greater than 10% of the population.
64. The method of any one of items 59-63, wherein the subject has previously been treated for an HSV (e.g., HSV-1 and/or HSV-2) infection with a different pharmaceutical composition.
65. The method of any one of items 59-64, further comprising administering a second dose of the pharmaceutical composition to the patient.
66. The method of any one of items 59-65, further comprising administering at least two doses of the pharmaceutical composition to the patient.
67. The method of any one of items 59-66, further comprising administering at least three doses of the pharmaceutical composition to the patient.
68. The method of any one of items 59-67, wherein the method is a method of inducing an anti-HSV (e.g., anti-HSV-1 and/or anti-HSV-2) immune response in the subject.
69. The method of item 68, wherein the immune response in the subject comprises an adaptive immune response.
70. The method of item 68 or item 69, wherein the immune response in the subject comprises a T-cell response.
71. The method of item 70, wherein the T-cell response is or comprises a CD4+ T cell response.
72. The method of item 70 or item 71, wherein the T-cell response is or comprises a CD8+ T cell response.
73. The method of any one of items 68-72, wherein the immune system response comprises a B-cell response.
74. The method of any one of items 68-73, wherein the immune system response comprises the production of antibodies directed against the one or more HSV (e.g., HSV-1 and/or HSV-2) antigens or fragments thereof.
75. The pharmaceutical composition of any one of items 52-58 for use in the treatment of an HSV (e.g., HSV-1 and/or HSV-2) infection.
76. The pharmaceutical composition of any one of items 52-58 for use in inducing an anti-HSV (e.g., anti-HSV-1 and/or anti-HSV-2) immune response in the subject.
77. Use of the pharmaceutical composition of any one of items 52-58 in the treatment of an HSV (e.g., HSV-1 and/or HSV-2) infection.
78. Use of the pharmaceutical composition of any one of items 52-58 in inducing an anti-HSV (e.g., anti-HSV-1 and/or anti-HSV-2) immune response in the subject.
79. A polypeptide comprising one or more of HSV (e.g., HSV-1 and/or HSV-2) antigens or fragments thereof.
80. The polypeptide of item 79, wherein the one or more HSV antigens or fragments thereof have at least 80% sequence identity with one or more sequences selected from SEQ ID NOs: 1-74 (Table 1) or a corresponding fragment thereof.
81. The polypeptide of item 79 or item 80, wherein the one or more HSV antigens or fragments thereof have at least 85%, 90%, 95%, or 100% sequence identity with one or more sequences selected from SEQ ID NOs: 1-74 (Table 1) or a corresponding fragment thereof.
82. The polypeptide of any one of items 79-81, wherein the one or more HSV antigens or fragments thereof have at least 80% sequence identity with one or more sequences selected from SEQ ID NOs: 1-30 and 74 (Table 2) or a corresponding fragment thereof.
83. The polypeptide of any one of items 79-81, wherein the one or more HSV antigens or fragments thereof have at least 85%, 90%, 95%, or 100% sequence identity with one or more sequences selected from SEQ ID NOs: 1-30 and 74 (Table 2) or a corresponding fragment thereof.
84. The polypeptide of any one of items 79-81, wherein the one or more HSV antigens or fragments thereof have at least 80% sequence identity with one or more sequences selected from SEQ ID NOs: 31-73 (Table 3) or a corresponding fragment thereof.
85. The polypeptide of any one of items 79-81, wherein the one or more HSV antigens or fragments thereof have at least 85%, 90%, 95%, or 100% sequence identity with one or more sequences selected from SEQ ID NOs: 31-73 (Table 3) or a corresponding fragment thereof.
86. A polynucleotide encoding one or more of HSV (e.g., HSV-1 and/or HSV-2) antigens or fragments thereof.
87. The polynucleotide of item 86, wherein the one or more HSV antigens or fragments thereof have at least 80% sequence identity with one or more sequences selected from SEQ ID NOs: 1-74 (Table 1) or a corresponding fragment thereof.
88. The polynucleotide of item 86 or item 87, wherein the one or more HSV antigens or fragments thereof have at least 85%, 90%, 95%, or 100% sequence identity with one or more sequences selected from SEQ ID NOs: 1-74 (Table 1) or a corresponding fragment thereof.
89. The polynucleotide of any one of items 86-88, wherein the one or more HSV antigens or fragments thereof have at least 80% sequence identity with one or more sequences selected from SEQ ID NOs: 1-30 and 74 (Table 2) or a corresponding fragment thereof.
90. The polynucleotide of any one of items 86-88, wherein the one or more HSV antigens or fragments thereof have at least 85%, 90%, 95%, or 100% sequence identity with one or more sequences selected from SEQ ID NOs: 1-30 and 74 (Table 2) or a corresponding fragment thereof.
91. The polynucleotide of any one of items 86-88, wherein the one or more HSV antigens or fragments thereof have at least 80% sequence identity with one or more sequences selected from SEQ ID NOs: 31-73 (Table 3) or a corresponding fragment thereof.
92. The polynucleotide of any one of items 86-88, wherein the one or more HSV antigens or fragments thereof have at least 85%, 90%, 95%, or 100% sequence identity with one or more sequences selected from SEQ ID NOs: 31-73 (Table 3) or a corresponding fragment thereof.
93. The polynucleotide of any one of items 86-92, wherein the polynucleotide is DNA or RNA.
94. A cell comprising a polypeptide of any one of items 79-85 and/or a polynucleotide of any one of items 86-93.
95. A cell comprising a polynucleotide of any one of items 86-93.
96. The cell of item 95, wherein the cell expresses the one or more HSV (e.g., HSV-1 and/or HSV-2) antigens or fragments thereof encoded by the polynucleotide.
97. A polyribonucleotide encoding a polypeptide comprising one or more HSV-2 antigens, wherein the one or more HSV-2 antigens comprise an RL2 polypeptide or antigenic fragment thereof, an RS1 polypeptide or antigenic fragment thereof, a UL54 polypeptide or antigenic fragment thereof, a UL29 polypeptide or antigenic fragment thereof, a UL39 polypeptide or antigenic fragment thereof, a UL49 polypeptide or antigenic fragment thereof, a UL9 polypeptide or antigenic fragment thereof, a UL30 polypeptide or antigenic fragment thereof, a UL40 polypeptide or antigenic fragment thereof, a UL5 polypeptide or antigenic fragment thereof, a UL52 polypeptide or antigenic fragment thereof, a UL1 polypeptide or antigenic fragment thereof, a UL19 polypeptide or antigenic fragment thereof, a UL21 polypeptide or antigenic fragment thereof, a UL27 polypeptide or antigenic fragment thereof, a UL46 polypeptide or antigenic fragment thereof, a UL47 polypeptide or antigenic fragment thereof, a UL48 polypeptide or antigenic fragment thereof, a UL25 polypeptide or antigenic fragment thereof, or a combination thereof.
98. A polyribonucleotide encoding a polypeptide comprising one or more HSV-2 antigens, wherein at least one HSV-2 antigen comprises an intermediate early protein or an antigenic fragment thereof.
99. A polyribonucleotide encoding a polypeptide comprising one or more HSV-2 antigens, wherein each of the one or more HSV-2 antigens comprises an intermediate early protein or an antigenic fragment thereof.
100. A polyribonucleotide encoding a polypeptide comprising one or more HSV-2 antigens, wherein at least one HSV-2 antigen comprises an early protein or an antigenic fragment thereof.
101. A polyribonucleotide encoding a polypeptide comprising one or more HSV-2 antigens, wherein each of the one or more HSV-2 antigens comprises an early protein or an antigenic fragment thereof.
102. A polyribonucleotide encoding a polypeptide comprising one or more HSV-2 antigens, wherein at least one HSV-2 antigen comprises a late protein or an antigenic fragment thereof.
103. A polyribonucleotide encoding a polypeptide comprising one or more HSV-2 antigens, wherein each of the one or more HSV-2 antigens comprises a late protein or an antigenic fragment thereof.
104. A polyribonucleotide comprising, in 5′ to 3′ order, nucleotide sequences that encode an HSV-1 gD secretory signal, an RL2 polypeptide or antigenic fragment thereof, a linker, an RL2 polypeptide or antigenic fragment thereof, a linker, an RS1 polypeptide or antigenic fragment thereof, a linker, a UL54 polypeptide or fragment thereof, a linker, and a MITD.
105. A polyribonucleotide encoding a polypeptide having an amino acid sequence comprising or consisting of

(SEQ ID NO: 197)
MGGAAARLGAVILFVVIVGLHGVRGCTDEIAPPLRCQSFPCLHPFCIPCM
KTWIPLRNTCPLCNTPVAYLIVGVTASGSFSTIPIVNDPRTRVEAEAAVR
AGTAVDFIWTGNPRTAPRSLSGGSGGGGSGGLPIAGVSSVVALAPYVNKT
VTGDCLPVLDMETGHIGAYVVLVDQTGNVADLLRAAAPAWSRRTLLPEHA
RNCVRPPDYPTPPASEWNSLWMTPVGNMLFDQGTLVGGGSGGGGSGGRAA
AWMRQVPDPEDVRVVILYSPLPGEDLAAGRAGGGPPPEWSAERGGLSCLL
AALGNRLCGPATAAWAGNWTGAPDVSALGAQGVLLLSTRDLAFAGAVEFL
GLLAGACDRRLIVVNAVRAADWPADGPVVSRQHAYLACEVLPAVQCAVRW
PAARDLRRTVLASGRVFGPGVFARVEAAHARLYPDAPPLRLCRGANVRYR
VRTRFGPDTLVPMSPREYRRAVLPALDGRAAASGGSGGGGSGGETLVAHG
PSLYRTFAANPRAASTAKAMRDCVLRQENLIEALASADETLAWCKMCIHH
NLPLRPQDPIIGTAAAVLENLATRLRPFLQCYLKARGLCGLDDLCSRRRL
SDIKDIASFVLVILARLANRVERGVSEIDYTTVGVGAGETMHFYIPGACM
AGLIEILDTHRQECSSRVCELTASHTIAPLYVHGKYFYCNSLFGGSGGGG
SGGIVGIVAGLAVLAVVVIGAVVATVMCRRKSSGGKGGSYSQAASSDSAQ
GSDVSLTA.

106. A polyribonucleotide comprising, in 5′ to 3′ order, nucleotide sequences that encode an HSV-1 gD secretory signal, an UL29 polypeptide or antigenic fragment thereof, a linker, an UL39 polypeptide or antigenic fragment thereof, a linker, an UL49 polypeptide or antigenic fragment thereof, a linker, a UL9 polypeptide or fragment thereof, a linker, and a MITD.
107. A polyribonucleotide encoding a polypeptide having an amino acid sequence comprising or consisting of

(SEQ ID NO: 198)
MGGAAARLGAVILFVVIVGLHGVRGREDIETIAFIKRFSLDYGAINFINL
APNNVSELAMYYMANQILRYCDHSTYFINTLTAVIAGSRRPPSVQAAAAW
APQGGAGLEAGARALMDSLDAHPGAWTSMFASCNLLRPVMAARPMVVLGL
SISKYYGMAGNDRVFQAGNWASLLGGKNACPLLIFDRTRKFVLGGSGGGG
SGGRTFGSAPRLTEDDFGLLNYALAEMRRLCLDLPPVPPNAYTPYHLREY
ATRLVNGFKPLVRRSARLYRILGVLVHLRIRTREASFEEWMRSKEVDLDF
GLTERLREHEAQLMILAQALNPYDCLIHSTPNTLVERGLQSALKYEEFYL
KRFGGHYMESVFQMYTRIAGFLAGGSGGGGSGGKMTRGAPKASATPATDP
ARGRRPAQADSAVLLDAPAPTASGRTKTPAQGLAKKLHFSTAPPSPTAPW
TPRVAGFNKRVFCAAVGGGSGGGGSGGLLNNYDVLVLDEVMSTLGQLYSP
TMQQLGRVDALMLRLLRTCPRIIAMDATANAQLVDFLCSLRGEKNVHVVI
GEYAMPGFSARRCLFLPRLGPEVLQAALRRRGPAGGAPPPDAPPDATFFG
ELEARLAGGDNVCIFSSTVSFAEVVARFCRQFTDRVLLLHSLTPPGDVTT
WGRYRVVIYTTVVTVGLSFDPPHFDSMFAYVKPMNYGPDMVSVYQSLGRV
RTLRKGELLIYMDGSGARSEPVGGSGGGGSGGIVGIVAGLAVLAVVVIGA
VVATVMCRRKSSGGKGGSYSQAASSDSAQGSDVSLTA.

108. A polyribonucleotide comprising, in 5′ to 3′ order, nucleotide sequences that encode an HSV-1 gD secretory signal, an UL30 polypeptide or antigenic fragment thereof, a linker, an UL30 polypeptide or antigenic fragment thereof, a linker, an UL40 polypeptide or antigenic fragment thereof, a linker, a UL5 polypeptide or fragment thereof, a linker, a UL5 polypeptide or fragment thereof, a linker, a UL52 polypeptide or fragment thereof, a linker, and a MITD.
109. A polyribonucleotide encoding a polypeptide having an amino acid sequence comprising or consisting of

(SEQ ID NO: 199)
MGGAAARLGAVILFVVIVGLHGVRGISCLLYDLSTTALEHILLFSLGSCD
LPESHLSDLASRGLPAPVVLEFDSEFEMLLAFMTFVKQYGPEFVTGYNII
NFDWPFVLTKLTEIYKVPLDGYGRMNGRGVFRVWDIGQSHFGGSGGGGSG
GGLLPCLHVAATVTTIGREMLLATRAYVHARWAEFDQLLADFPEAAGMRA
PGPYSMGGSGGGGSGGTSQCPDINHLRSLSILNRWLETELVFVGDEEDVS
KLSEGELGFYRFLFAFLSAADDLVTENLGGLSGLFEQKDILHYYVEQECI
EVVHSRVYNIIQLVLFHNNDQARRAYVARTINHPAIRVKVDWLEARVREC
DSIPEKFILMILIEGVFFAASFAAIAYLRTNNLLRGGSGGGGSGGHEFGN
LMKVLEYGLPITEEHMQFVDRFVVPESYITNPANLPGWTRLFSSHKEVSA
YMAKLHAYLKVTREGEFVVFTLPVLTFVSVKEFDEYRRLGGSGGGGSGGE
LFGEVFESAPFSTYVDNVIFRGCELLTGSPRGGLMSVALQTDNYTLMGYT
YTRVFAFAEELRRRHATAGVAEFLEESPLPYIVLRDQHGFMSVVNTNIGG
SGGGGSGGSVAAPVEVTALYATDGCVITSSLALLTNCLLGAEPLYIFSYD
AYRSDAPNGPTGAPTEQERFEGSRALYRDAGGLNGDSFRVTFCLLGTEVG
VTHHPKGRTRPMFVCRFERADDVAVLQDALGRGTPLLPAHVTATLDLEAT
FALHANIIMALTVAIVHNAPARIGSGSTAPLYEPGESMRSVVGGSGGGGS
GGIVGIVAGLAVLAVVVIGAVVATVMCRRKSSGGKGGSYSQAASSDSAQG
SDVSLTA.

110. A polyribonucleotide comprising, in 5′ to 3′ order, nucleotide sequences that encode an HSV-1 gD secretory signal, an UL1 polypeptide or antigenic fragment thereof, a linker, an UL19 polypeptide or antigenic fragment thereof, a linker, an UL21 polypeptide or antigenic fragment thereof, a linker, a UL27 polypeptide or fragment thereof, a linker, a UL27 polypeptide or fragment thereof, a linker, a UL46 polypeptide or fragment thereof, a linker, a UL47 polypeptide or fragment thereof, a linker, a UL25 polypeptide or fragment thereof, a linker, a UL48 polypeptide or fragment thereof, a linker, and a MITD.
111. A polyribonucleotide encoding a polypeptide having an amino acid sequence comprising or consisting of

(SEQ ID NO: 200)
MGGAAARLGAVILFVVIVGLHGVRGRTPADDVSWRYEAPSVIDYARIDGI
FLRYHCPGLDTFLWDRHAQRAYLVNPFLFAAGFLEDLSHSVFPADTQETT
GGSGGGGSGGDGRLLHNTQARAADAADDRPHRPADWTVHHKIYYYVLVPA
FSRGRCCTAGVRFDRVYATLQNMVVPEIAPGEECPSDPVTDPAHPLHPAN
LVANTVKRMFHNGGSGGGGSGGSPTQKLAVYYYLIHRERRMSPFPALVRL
VGRYIQRHGLYVPAPDEPTLADAMNGLGGSGGGGSGGNYTEGIAVVFKEN
IAPYKFKATMYYKDVTVSQVWFGHRYSQFMGIFEDRAPVPFEEVGGSGGG
GSGGSVYPYDEFVLATGDFVYMSPFYGYREGSHGGSGGGGSGGGLASDPH
YDYIRHYASAAKQALGEVELSGGQLSRAILAQYWKYLQTVVPSGLDIPDD
PAGDCDPSLHVLLRPTLLPKLLVRAPFKSGAAAAKYAAAVAGLRDAAHRL
QQYMFFMRPADPSRPSTDTALRLSELLAYVSVLYHWASWMLWTADKYVGG
SGGGGSGGGPDAAVFRSSLGSLLYWPGVRALLGRDCRVAARYAGRMTYIA
TGALLARFNPGAVKCVLPREAAFAGRVLGGSGGGGSGGFLWEDQTLLRAT
ANTITALAVLRRLLANGNVYADRLDNRLQLGMLIPGAVPAEAIARGASGL
DSGAIKSGDNNLEALCVNYVLPLYQADPTVELTQLFPGLAALCLGGSGGG
GSGGALFNRLLDDLGFSAGPALCTMLDTWNEDLFSGFPTNADMYRECKFL
STLPSDVIDWGDAHVPERSPIDIRAHGDVAFPTLPATRDELPSYYEAMAQ
FFRGELRAGGSGGGGSGGIVGIVAGLAVLAVVVIGAVVATVMCRRKSSGG
KGGSYSQAASSDSAQGSDVSLTA.

112. A polyribonucleotide comprising, in 5′ to 3′ order, nucleotide sequences that encode an HSV-1 gD secretory signal, an UL54 polypeptide or antigenic fragment thereof, a linker, an RS1 polypeptide or antigenic fragment thereof, a linker, an RL2 polypeptide or antigenic fragment thereof, a linker, a RL2 polypeptide or fragment thereof, a linker, and a MITD.
113. A polyribonucleotide encoding a polypeptide having an amino acid sequence comprising or consisting of

(SEQ ID NO: 201)
MGGAAARLGAVILFVVIVGLHGVRGETLVAHGPSLYRTFAANPRAASTAK
AMRDCVLRQENLIEALASADETLAWCKMCIHHNLPLRPQDPIIGTAAAVL
ENLATRLRPFLQCYLKARGLCGLDDLCSRRRLSDIKDIASFVLVILARLA
NRVERGVSEIDYTTVGVGAGETMHFYIPGACMAGLIEILDTHRQECSSRV
CELTASHTIAPLYVHGKYFYCNSLFGGSGGGGSGGRAAAWMRQVPDPEDV
RVVILYSPLPGEDLAAGRAGGGPPPEWSAERGGLSCLLAALGNRLCGPAT
AAWAGNWTGAPDVSALGAQGVLLLSTRDLAFAGAVEFLGLLAGACDRRLI
VVNAVRAADWPADGPVVSRQHAYLACEVLPAVQCAVRWPAARDLRRTVLA
SGRVFGPGVFARVEAAHARLYPDAPPLRLCRGANVRYRVRTRFGPDTLVP
MSPREYRRAVLPALDGRAAASGGSGGGGSGGCTDEIAPPLRCQSFPCLHP
FCIPCMKTWIPLRNTCPLCNTPVAYLIVGVTASGSFSTIPIVNDPRTRVE
AEAAVRAGTAVDFIWTGNPRTAPRSLSGGSGGGGSGGLPIAGVSSVVALA
PYVNKTVTGDCLPVLDMETGHIGAYVVLVDQTGNVADLLRAAAPAWSRRT
LLPEHARNCVRPPDYPTPPASEWNSLWMTPVGNMLFDQGTLVGGGSGGGG
SGGIVGIVAGLAVLAVVVIGAVVATVMCRRKSSGGKGGSYSQAASSDSAQ
GSDVSLTA.

114. A polyribonucleotide comprising, in 5′ to 3′ order, nucleotide sequences that encode an HSV-1 gD secretory signal, an UL9 polypeptide or antigenic fragment thereof, a linker, an UL49 polypeptide or antigenic fragment thereof, a linker, an UL39 polypeptide or antigenic fragment thereof, a linker, a UL29 polypeptide or fragment thereof, a linker, and a MITD.
115. A polyribonucleotide encoding a polypeptide having an amino acid sequence comprising or consisting of

(SEQ ID NO: 202)
MGGAAARLGAVILFVVIVGLHGVRGLLNNYDVLVLDEVMSTLGQLYSPTM
QQLGRVDALMLRLLRTCPRIIAMDATANAQLVDFLCSLRGEKNVHVVIGE
YAMPGFSARRCLFLPRLGPEVLQAALRRRGPAGGAPPPDAPPDATFFGEL
EARLAGGDNVCIFSSTVSFAEVVARFCRQFTDRVLLLHSLTPPGDVTTWG
RYRVVIYTTVVTVGLSFDPPHFDSMFAYVKPMNYGPDMVSVYQSLGRVRT
LRKGELLIYMDGSGARSEPVGGSGGGGSGGKMTRGAPKASATPATDPARG
RRPAQADSAVLLDAPAPTASGRTKTPAQGLAKKLHFSTAPPSPTAPWTPR
VAGFNKRVFCAAVGGGSGGGGSGGRTFGSAPRLTEDDFGLLNYALAEMRR
LCLDLPPVPPNAYTPYHLREYATRLVNGFKPLVRRSARLYRILGVLVHLR
IRTREASFEEWMRSKEVDLDFGLTERLREHEAQLMILAQALNPYDCLIHS
TPNTLVERGLQSALKYEEFYLKRFGGHYMESVFQMYTRIAGFLAGGSGGG
GSGGREDIETIAFIKRFSLDYGAINFINLAPNNVSELAMYYMANQILRYC
DHSTYFINTLTAVIAGSRRPPSVQAAAAWAPQGGAGLEAGARALMDSLDA
HPGAWTSMFASCNLLRPVMAARPMVVLGLSISKYYGMAGNDRVFQAGNWA
SLLGGKNACPLLIFDRTRKFVLGGSGGGGSGGIVGIVAGLAVLAVVVIGA
VVATVMCRRKSSGGKGGSYSQAASSDSAQGSDVSLTA.

116. A polyribonucleotide comprising, in 5′ to 3′ order, nucleotide sequences that encode an HSV-1 gD secretory signal, an UL52 polypeptide or antigenic fragment thereof, a linker, an UL5 polypeptide or antigenic fragment thereof, a linker, an UL5 polypeptide or antigenic fragment thereof, a linker, a UL40 polypeptide or fragment thereof, a linker, a UL30 polypeptide or fragment thereof, a linker, a UL30 polypeptide or fragment thereof, a linker, and a MITD.
117. A polyribonucleotide encoding a polypeptide having an amino acid sequence comprising or consisting of

(SEQ ID NO: 203)
MGGAAARLGAVILFVVIVGLHGVRGSVAAPVEVTALYATDGCVITSSLAL
LTNCLLGAEPLYIFSYDAYRSDAPNGPTGAPTEQERFEGSRALYRDAGGL
NGDSFRVTFCLLGTEVGVTHHPKGRTRPMFVCRFERADDVAVLQDALGRG
TPLLPAHVTATLDLEATFALHANIIMALTVAIVHNAPARIGSGSTAPLYE
PGESMRSVVGGSGGGGSGGHEFGNLMKVLEYGLPITEEHMQFVDRFVVPE
SYITNPANLPGWTRLFSSHKEVSAYMAKLHAYLKVTREGEFVVFTLPVLT
FVSVKEFDEYRRLGGSGGGGSGGELFGEVFESAPFSTYVDNVIFRGCELL
TGSPRGGLMSVALQTDNYTLMGYTYTRVFAFAEELRRRHATAGVAEFLEE
SPLPYIVLRDQHGFMSVVNTNIGGSGGGGSGGTSQCPDINHLRSLSILNR
WLETELVFVGDEEDVSKLSEGELGFYRFLFAFLSAADDLVTENLGGLSGL
FEQKDILHYYVEQECIEVVHSRVYNIIQLVLFHNNDQARRAYVARTINHP
AIRVKVDWLEARVRECDSIPEKFILMILIEGVFFAASFAAIAYLRTNNLL
RGGSGGGGSGGISCLLYDLSTTALEHILLFSLGSCDLPESHLSDLASRGL
PAPVVLEFDSEFEMLLAFMTFVKQYGPEFVTGYNIINFDWPFVLTKLTEI
YKVPLDGYGRMNGRGVFRVWDIGQSHFGGSGGGGSGGGLLPCLHVAATVT
TIGREMLLATRAYVHARWAEFDQLLADFPEAAGMRAPGPYSMGGSGGGGS
GGIVGIVAGLAVLAVVVIGAVVATVMCRRKSSGGKGGSYSQAASSDSAQG
SDVSLTA.

118. A polyribonucleotide comprising, in 5′ to 3′ order, nucleotide sequences that encode an HSV-1 gD secretory signal, an UL48 polypeptide or antigenic fragment thereof, a linker, an UL25 polypeptide or antigenic fragment thereof, a linker, an UL47 polypeptide or antigenic fragment thereof, a linker, a UL46 polypeptide or fragment thereof, a linker, a UL27 polypeptide or fragment thereof, a linker, a UL27 polypeptide or fragment thereof, a linker, a UL21 polypeptide or fragment thereof, a linker, a UL19 polypeptide or fragment thereof, a linker, a UL1 polypeptide or fragment thereof, a linker, and a MITD.
119. A polyribonucleotide encoding a polypeptide having an amino acid sequence comprising or consisting of

(SEQ ID NO: 204)
MGGAAARLGAVILFVVIVGLHGVRGALFNRLLDDLGFSAGPALCTMLDTW
NEDLFSGFPTNADMYRECKFLSTLPSDVIDWGDAHVPERSPIDIRAHGDV
AFPTLPATRDELPSYYEAMAQFFRGELRAGGSGGGGSGGFLWEDQTLLRA
TANTITALAVLRRLLANGNVYADRLDNRLQLGMLIPGAVPAEAIARGASG
LDSGAIKSGDNNLEALCVNYVLPLYQADPTVELTQLFPGLAALCLGGSGG
GGSGGGPDAAVFRSSLGSLLYWPGVRALLGRDCRVAARYAGRMTYIATGA
LLARFNPGAVKCVLPREAAFAGRVLGGSGGGGSGGGLASDPHYDYIRHYA
SAAKQALGEVELSGGQLSRAILAQYWKYLQTVVPSGLDIPDDPAGDCDPS
LHVLLRPTLLPKLLVRAPFKSGAAAAKYAAAVAGLRDAAHRLQQYMFFMR
PADPSRPSTDTALRLSELLAYVSVLYHWASWMLWTADKYVGGSGGGGSGG
NYTEGIAVVFKENIAPYKFKATMYYKDVTVSQVWFGHRYSQFMGIFEDRA
PVPFEEVGGSGGGGSGGSVYPYDEFVLATGDFVYMSPFYGYREGSHGGSG
GGGSGGSPTQKLAVYYYLIHRERRMSPFPALVRLVGRYIQRHGLYVPAPD
EPTLADAMNGLGGSGGGGSGGDGRLLHNTQARAADAADDRPHRPADWTVH
HKIYYYVLVPAFSRGRCCTAGVRFDRVYATLQNMVVPEIAPGEECPSDPV
TDPAHPLHPANLVANTVKRMFHNGGSGGGGSGGRTPADDVSWRYEAPSVI
DYARIDGIFLRYHCPGLDTFLWDRHAQRAYLVNPFLFAAGFLEDLSHSVF
PADTQETTGGSGGGGSGGIVGIVAGLAVLAVVVIGAVVATVMCRRKSSGG
KGGSYSQAASSDSAQGSDVSLTA.

120. A polyribonucleotide comprising, in 5′ to 3′ order, nucleotide sequences that encode an HSV-2 gD secretory signal, an RL2 polypeptide or antigenic fragment thereof, a linker, an RL2 polypeptide or antigenic fragment thereof, a linker, an RS1 polypeptide or antigenic fragment thereof, a linker, a UL54 polypeptide or fragment thereof, a linker, and a MITD.
121. A polyribonucleotide encoding a polypeptide having an amino acid sequence comprising or consisting of

(SEQ ID NO: 205)
MGRLTSGVGTAALLVVAVGLRVVCACTDEIAPPLRCQSFPCLHPFCIPCM
KTWIPLRNTCPLCNTPVAYLIVGVTASGSFSTIPIVNDPRTRVEAEAAVR
AGTAVDFIWTGNPRTAPRSLSGGSGGGGSGGLPIAGVSSVVALAPYVNKT
VTGDCLPVLDMETGHIGAYVVLVDQTGNVADLLRAAAPAWSRRTLLPEHA
RNCVRPPDYPTPPASEWNSLWMTPVGNMLFDQGTLVGGGSGGGGSGGRAA
AWMRQVPDPEDVRVVILYSPLPGEDLAAGRAGGGPPPEWSAERGGLSCLL
AALGNRLCGPATAAWAGNWTGAPDVSALGAQGVLLLSTRDLAFAGAVEFL
GLLAGACDRRLIVVNAVRAADWPADGPVVSRQHAYLACEVLPAVQCAVRW
PAARDLRRTVLASGRVFGPGVFARVEAAHARLYPDAPPLRLCRGANVRYR
VRTRFGPDTLVPMSPREYRRAVLPALDGRAAASGGSGGGGSGGETLVAHG
PSLYRTFAANPRAASTAKAMRDCVLRQENLIEALASADETLAWCKMCIHH
NLPLRPQDPIIGTAAAVLENLATRLRPFLQCYLKARGLCGLDDLCSRRRL
SDIKDIASFVLVILARLANRVERGVSEIDYTTVGVGAGETMHFYIPGACM
AGLIEILDTHRQECSSRVCELTASHTIAPLYVHGKYFYCNSLFGGSGGGG
SGGIVGIVAGLAVLAVVVIGAVVATVMCRRKSSGGKGGSYSQAASSDSAQ
GSDVSLTA.

122. A polyribonucleotide comprising, in 5′ to 3′ order, nucleotide sequences that encode an HSV-2 gD secretory signal, an UL54 polypeptide or antigenic fragment thereof, a linker, an RS1 polypeptide or antigenic fragment thereof, a linker, an RL2 polypeptide or antigenic fragment thereof, a linker, a RL2 polypeptide or fragment thereof, a linker, and a MITD.
123. A polyribonucleotide encoding a polypeptide having an amino acid sequence comprising or consisting of

(SEQ ID NO: 206)
MGRLTSGVGTAALLVVAVGLRVVCAETLVAHGPSLYRTFAANPRAASTAK
AMRDCVLRQENLIEALASADETLAWCKMCIHHNLPLRPQDPIIGTAAAVL
ENLATRLRPFLQCYLKARGLCGLDDLCSRRRLSDIKDIASFVLVILARLA
NRVERGVSEIDYTTVGVGAGETMHFYIPGACMAGLIEILDTHRQECSSRV
CELTASHTIAPLYVHGKYFYCNSLFGGSGGGGSGGRAAAWMRQVPDPEDV
RVVILYSPLPGEDLAAGRAGGGPPPEWSAERGGLSCLLAALGNRLCGPAT
AAWAGNWTGAPDVSALGAQGVLLLSTRDLAFAGAVEFLGLLAGACDRRLI
VVNAVRAADWPADGPVVSRQHAYLACEVLPAVQCAVRWPAARDLRRTVLA
SGRVFGPGVFARVEAAHARLYPDAPPLRLCRGANVRYRVRTRFGPDTLVP
MSPREYRRAVLPALDGRAAASGGSGGGGSGGCTDEIAPPLRCQSFPCLHP
FCIPCMKTWIPLRNTCPLCNTPVAYLIVGVTASGSFSTIPIVNDPRTRVE
AEAAVRAGTAVDFIWTGNPRTAPRSLSGGSGGGGSGGLPIAGVSSVVALA
PYVNKTVTGDCLPVLDMETGHIGAYVVLVDQTGNVADLLRAAAPAWSRRT
LLPEHARNCVRPPDYPTPPASEWNSLWMTPVGNMLFDQGTLVGGGSGGGG
SGGIVGIVAGLAVLAVVVIGAVVATVMCRRKSSGGKGGSYSQAASSDSAQ
GSDVSLTA.

124. A polyribonucleotide encoding a polypeptide, wherein the polypeptide comprises one or more herpes simplex virus (HSV) antigens or antigenic fragments thereof.
125. The polyribonucleotide of item 124, wherein the one or more HSV antigens or antigenic fragments thereof comprise:
   (i) HSV-1 antigens or antigenic fragments thereof,
   (ii) HSV-2 antigens or antigenic fragments thereof, or
   (iii) a combination thereof.
126. The polyribonucleotide of any one of items 124 to 125, wherein the polypeptide comprises a single HSV antigen or antigenic fragment thereof.
127. The polyribonucleotide of any one of items 124 to 126, wherein the polypeptide comprises a single HSV antigen.
128. The polyribonucleotide of item 126, wherein the polypeptide comprises a single HSV antigenic fragment.
129. The polyribonucleotide of any one of items 124 to 125, wherein the polypeptide comprises two or more HSV antigens or antigenic fragments thereof.
130. The polyribonucleotide of item 129, wherein the polypeptide comprises two or more HSV antigens.
131. The polyribonucleotide of item 129, wherein the polypeptide comprises two or more HSV antigenic fragments, wherein the two or more HSV antigenic fragments are each a fragment of a different HSV antigen.
132. The polyribonucleotide of items 129 or 131, wherein the polypeptide comprises two or more HSV antigenic fragments, wherein at least two of the HSV antigenic fragments are a fragment from the same HSV antigen.
133. The polyribonucleotide of any one of items 124 to 125, wherein the polypeptide comprises three or more HSV antigens or antigenic fragments thereof.
134. The polyribonucleotide of any one of items 124 to 125, wherein the polypeptide comprises four or more HSV antigens or antigenic fragments thereof.
135. The polyribonucleotide of any one of items 124 to 126, wherein the polypeptide does not comprise a full length HSV antigen.
136. The polyribonucleotide of any one of items 124 to 135, wherein the one or more HSV antigens or antigenic fragments thereof comprise one or more T cell antigens or antigenic fragments thereof.
137. The polyribonucleotide of any one of items 124 to 135, wherein the one or more HSV antigens or antigenic fragments thereof comprise one or more B cell antigens or antigenic fragments thereof.
138. The polyribonucleotide of any one of items 124 to 136, wherein the one or more HSV antigens or antigenic fragments thereof have at least 80% sequence identity with one or more sequences selected from SEQ ID NOs: 1-74 or an antigenic fragment thereof.
139. The polyribonucleotide of item 138, wherein the one or more HSV antigens or antigenic fragments thereof have at least 85%, 90%, 95%, or 100% sequence identity with one or more sequences selected from SEQ ID NOs: 1-74 or an antigenic fragment thereof.
140. The polyribonucleotide of any one of items 124 to 135, wherein the polypeptide comprises one or more HSV-2 antigens or antigenic fragments thereof comprising or consisting of an amino acid sequence selected from SEQ ID NO: 174-196.
141. The polyribonucleotide of any one of items 138 to 140, wherein the one or more HSV antigens or antigenic fragments thereof comprise:
   (i) one or more HSV RS1 polypeptides or antigenic fragments thereof,
   (ii) one or more HSV RL2 polypeptides or antigenic fragments thereof,
   (iii) one or more HSV UL1 polypeptides or antigenic fragments thereof,
   (iv) one or more HSV UL5 polypeptides or antigenic fragments thereof,
   (v) one or more HSV UL9 polypeptides or antigenic fragments thereof,
   (vi) one or more HSV UL19 polypeptides or antigenic fragments thereof,
   (vii) one or more HSV UL21 polypeptides or antigenic fragments thereof,
   (viii) one or more HSV UL25 polypeptides or antigenic fragments thereof,
   (ix) one or more HSV UL27 polypeptides or antigenic fragments thereof,
   (x) one or more HSV UL29 polypeptides or antigenic fragments thereof,
   (xi) one or more HSV UL30 polypeptides or antigenic fragments thereof,
   (xii) one or more HSV UL39 polypeptides or antigenic fragments thereof,
   (xiii) one or more HSV UL40 polypeptides or antigenic fragments thereof,
   (xiv) one or more HSV UL46 polypeptides or antigenic fragments thereof,
   (xv) one or more HSV UL47 polypeptides or antigenic fragments thereof,
   (xvi) one or more HSV UL48 polypeptides or antigenic fragments thereof,
   (xvii) one or more HSV UL49 polypeptides or antigenic fragments thereof,
   (xviii) one or more HSV UL52 polypeptides or antigenic fragments thereof,
   (xix) one or more HSV UL54 polypeptides or antigenic fragments thereof, or
   (xx) a combination thereof.
142. The polyribonucleotide of any one of items 138 to 141, wherein the polypeptide comprises one or more HSV antigenic fragments, and the one or more HSV antigenic fragments comprise:
   (i) one or more HSV RS1 polypeptide antigenic fragments,
   (ii) one or more HSV RL2 polypeptide antigenic fragments,
   (iii) one or more HSV UL1 polypeptide antigenic fragments,
   (iv) one or more HSV UL5 polypeptide antigenic fragments,
   (v) one or more HSV UL9 polypeptide antigenic fragments,
   (vi) one or more HSV UL19 polypeptide antigenic fragments,
   (vii) one or more HSV UL21 polypeptide antigenic fragments,
   (viii) one or more HSV UL25 polypeptide antigenic fragments,
   (ix) one or more HSV UL27 polypeptide antigenic fragments,
   (x) one or more HSV UL29 polypeptide antigenic fragments,
   (xi) one or more HSV UL30 polypeptide antigenic fragments,
   (xii) one or more HSV UL39 polypeptide antigenic fragments,
   (xiii) one or more HSV UL40 polypeptide antigenic fragments,
   (xiv) one or more HSV UL46 polypeptide antigenic fragments,
   (xv) one or more HSV UL47 polypeptide antigenic fragments,
   (xvi) one or more HSV UL48 polypeptide antigenic fragments,
   (xvii) one or more HSV UL49 polypeptide antigenic fragments,
   (xviii) one or more HSV UL52 polypeptide antigenic fragments,
   (xix) one or more HSV UL54 polypeptide antigenic fragments, or
   (xx) a combination thereof.
143. The polyribonucleotide of any one of items 138 to 142, wherein the polypeptide comprises one or more HSV RL2 polypeptides or antigenic fragments thereof, one or more HSV RS1 polypeptides or antigenic fragments thereof, and one or more HSV UL54 polypeptides or antigenic fragments thereof.
144. The polyribonucleotide of item 143, wherein the polypeptide comprises an HSV-1 gD secretory signal, one or more RL2 polypeptides or antigenic fragments thereof, one or more RS1 polypeptides or antigenic fragments thereof, one or more UL54 polypeptides or antigenic fragments thereof, and a MITD.
145. The polyribonucleotide of items 143 or 144, wherein the polypeptide comprises, in N-terminus to C-terminus order, nucleotide sequences that encode an HSV-1 gD secretory signal, an RL2 polypeptide or antigenic fragment thereof, a linker, an RL2 polypeptide or antigenic fragment thereof, a linker, an RS1 polypeptide or antigenic fragment thereof, a linker, a UL54 polypeptide or antigenic fragment thereof, a linker, and a MITD.
146. The polyribonucleotide of item 145, wherein the polypeptide comprises or consists of an amino acid sequence according to SEQ ID NO: 197.
147. The polyribonucleotide of items 143 or 144, wherein the polypeptide comprises, in N-terminus to C-terminus order, nucleotide sequences that encode an HSV-1 gD secretory signal, an UL54 polypeptide or antigenic fragment thereof, a linker, an RS1 polypeptide or antigenic fragment thereof, a linker, an RL2 polypeptide or antigenic fragment thereof, a linker, a RL2 polypeptide or antigenic fragment thereof, a linker, and a MITD.
148. The polyribonucleotide of item 147, wherein the polypeptide comprises or consists of an amino acid sequence according to SEQ ID NO: 201.
149. The polyribonucleotide of items 143 or 144, wherein the polypeptide comprises, in N-terminus to C-terminus order, nucleotide sequences that encode an HSV-2 gD secretory signal, an RL2 polypeptide or antigenic fragment thereof, a linker, an RL2 polypeptide or antigenic fragment thereof, a linker, an RS1 polypeptide or antigenic fragment thereof, a linker, a UL54 polypeptide or antigenic fragment thereof, a linker, and a MITD.
150. The polyribonucleotide of item 149, wherein the polypeptide comprises or consists of an amino acid sequence according to SEQ ID NO: 205.
151. The polyribonucleotide of any one of items 138 to 142, wherein the polypeptide comprises one or more HSV UL29 polypeptides or antigenic fragments thereof, one or more HSV UL39 polypeptides or antigenic fragments thereof, one or more HSV UL49 polypeptides or antigenic fragments thereof, and one or more HSV UL9 polypeptides or antigenic fragments thereof.
152. The polyribonucleotide of item 151, wherein the polypeptide comprises an HSV-1 gD secretory signal, one or more HSV UL29 polypeptides or antigenic fragments thereof, one or more HSV UL39 polypeptides or antigenic fragments thereof, one or more HSV UL49 polypeptides or antigenic fragments thereof, one or more HSV UL9 polypeptides or antigenic fragments thereof, and a MITD.
153. The polyribonucleotide of items 151 or 152, wherein the polypeptide comprises, in N-terminus to C-terminus order, nucleotide sequences that encode an HSV-1 gD secretory signal, an UL29 polypeptide or antigenic fragment thereof, a linker, an UL39 polypeptide or antigenic fragment thereof, a linker, an UL49 polypeptide or antigenic fragment thereof, a linker, a UL9 polypeptide or antigenic fragment thereof, a linker, and a MITD.
154. The polyribonucleotide of item 153, wherein the polypeptide comprises or consists of an amino acid sequence according to SEQ ID NO: 198.
155. The polyribonucleotide of items 151 or 152, wherein the polypeptide comprises, in N-terminus to C-terminus order, nucleotide sequences that encode an HSV-1 gD secretory signal, an UL9 polypeptide or antigenic fragment thereof, a linker, an UL49 polypeptide or antigenic fragment thereof, a linker, an UL39 polypeptide or antigenic fragment thereof, a linker, a UL29 polypeptide or antigenic fragment thereof, a linker, and a MITD.
156. The polyribonucleotide of item 155, wherein the polypeptide comprises or consists of an amino acid sequence according to SEQ ID NO: 202.
157. The polyribonucleotide of any one of items 138 to 142, wherein the polypeptide comprises one or more HSV UL30 polypeptides or antigenic fragments thereof, one or more HSV UL40 polypeptides or antigenic fragments thereof, one or more HSV UL5 polypeptides or antigenic fragments thereof, and one or more HSV UL52 polypeptides or antigenic fragments thereof.
158. The polyribonucleotide of item 157, wherein the polypeptide comprises an HSV-1 gD secretory signal, one or more HSV UL30 polypeptides or antigenic fragments thereof, one or more HSV UL40 polypeptides or antigenic fragments thereof, one or more HSV UL5 polypeptides or antigenic fragments thereof, one or more HSV UL52 polypeptides or antigenic fragments thereof, and a MITD.
159. The polyribonucleotide of items 157 or 158, wherein the polypeptide comprises, in N-terminus to C-terminus order, nucleotide sequences that encode an HSV-1 gD secretory signal, an UL30 polypeptide or antigenic fragment thereof, a linker, an UL30 polypeptide or antigenic fragment thereof, a linker, an UL40 polypeptide or antigenic fragment thereof, a linker, a UL5 polypeptide or antigenic fragment thereof, a linker, a UL5 polypeptide or antigenic fragment thereof, a linker, a UL52 polypeptide or antigenic fragment thereof, a linker, and a MITD.
160. The polyribonucleotide of item 159, wherein the polypeptide comprises or consists of an amino acid sequence according to SEQ ID NO: 199.
161. The polyribonucleotide of items 157 or 158, wherein the polypeptide comprises, in N-terminus to C-terminus order, nucleotide sequences that encode an HSV-1 gD secretory signal, an UL52 polypeptide or antigenic fragment thereof, a linker, an UL5 polypeptide or antigenic fragment thereof, a linker, an UL5 polypeptide or antigenic fragment thereof, a linker, a UL40 polypeptide or antigenic fragment thereof, a linker, a UL30 polypeptide or antigenic fragment thereof, a linker, a UL30 polypeptide or antigenic fragment thereof, a linker, and a MITD.
162. The polyribonucleotide of item 161, wherein the polypeptide comprises or consists of an amino acid sequence according to SEQ ID NO: 203.
163. The polyribonucleotide of any one of items 138 to 142, wherein the polypeptide comprises one or more HSV UL1 polypeptides or antigenic fragments thereof, one or more HSV UL19 polypeptides or antigenic fragments thereof, one or more HSV UL21 polypeptides or antigenic fragments thereof, one or more HSV UL27 polypeptides or antigenic fragments thereof, one or more HSV UL46 polypeptides or antigenic fragments thereof, one or more HSV UL47 polypeptides or antigenic fragments thereof, one or more UL48 polypeptides or antigenic fragments thereof, and one or more HSV UL25 polypeptides or antigenic fragments thereof.
164. The polyribonucleotide of item 163, wherein the polypeptide comprises an HSV-1 gD secretory signal, one or more HSV UL1 polypeptides or antigenic fragments thereof, one or more HSV UL19 polypeptides or antigenic fragments thereof, one or more HSV UL21 polypeptides or antigenic fragments thereof, one or more HSV UL27 polypeptides or antigenic fragments thereof, one or more HSV UL46 polypeptides or antigenic fragments thereof, one or more HSV UL47 polypeptides or antigenic fragments thereof, one or more UL48 polypeptides or antigenic fragments thereof, one or more HSV UL25 polypeptides or antigenic fragments thereof, and a MITD.
165. The polyribonucleotide of items 163 or 164, wherein the polypeptide comprises, in N-terminus to C-terminus order, nucleotide sequences that encode an HSV-1 gD secretory signal, an HSV UL1 polypeptide or antigenic fragment thereof, a linker, an HSV UL19 polypeptide or antigenic fragment thereof, a linker, an HSV UL21 polypeptide or antigenic fragment thereof, a linker, a HSV UL27 polypeptide or antigenic fragment thereof, a linker, a HSV UL27 polypeptide or antigenic fragment thereof, a linker, a HSV UL46 polypeptide or antigenic fragment thereof, a linker, a HSV UL47 polypeptide or antigenic fragment thereof, a linker, a HSV UL25 polypeptide or antigenic fragment thereof, a linker, a HSV UL48 polypeptide or antigenic fragment thereof, a linker, and a MITD.
166. The polyribonucleotide of item 165, wherein the polypeptide comprises or consists of an amino acid sequence according to SEQ ID NO: 200.
167. The polyribonucleotide of item 163 or 164, wherein the polypeptide comprises, in N-terminus to C-terminus order, nucleotide sequences that encode an HSV-1 gD secretory signal, an HSV UL48 polypeptide or antigenic fragment thereof, a linker, an HSV UL25 polypeptide or antigenic fragment thereof, a linker, an HSV UL47 polypeptide or antigenic fragment thereof, a linker, a HSV UL46 polypeptide or antigenic fragment thereof, a linker, a HSV UL27 polypeptide or antigenic fragment thereof, a linker, a HSV UL27 polypeptide or antigenic fragment thereof, a linker, a HSV UL21 polypeptide or antigenic fragment thereof, a linker, a HSV UL19 polypeptide or antigenic fragment thereof, a linker, a HSV UL1 polypeptide or antigenic fragment thereof, a linker, and a MITD.
168. The polyribonucleotide of item 167, wherein the polypeptide comprises or consists of an amino acid sequence according to SEQ ID NO: 204.
169. The polyribonucleotide of any one of items 124 to 137, wherein the one or more HSV antigens or antigenic fragments thereof comprise one or more HSV glycoproteins.
170. The polyribonucleotide of any one of items 169, wherein the one or more HSV glycoproteins comprise an HSV glycoprotein B (gB), an HSV glycoprotein E (gE), an HSV glycoprotein G (gG), an HSV glycoprotein H (gH), an HSV glycoprotein I (gI), an HSV glycoprotein L (gL), or a combination thereof.
171. The polyribonucleotide of any one of items 169 to 170, wherein the polypeptide comprises a single HSV antigen.
172. The polyribonucleotide of any one of items 169 to 171, wherein the single HSV antigen is an HSV glycoprotein.
173. The polyribonucleotide of any one of items 169 to 172, wherein the HSV glycoprotein is a full-length HSV glycoprotein.
174. The polyribonucleotide of any one of items 169 to 173, wherein the HSV glycoprotein is an HSV gB, an HSV gE, an HSV gG, an HSV gH, an HSV gI, and an HSV gL.
175. The polyribonucleotide of any one of items 170 to 174, wherein the HSV glycoprotein is HSV-2 gB.
176. The polyribonucleotide of item 175, wherein the HSV-2 gB is or comprises an amino acid sequence that is at least 80%, 85%, 90%, 91%, 92%, 93%, 94%, 95%, 96%, 97%, 98%, or 99% identical to SEQ ID NOs: 7, 8, 9, or 74.
177. The polyribonucleotide of any one of items 175 to 176, wherein the HSV-2 gB consists or comprises an amino acid sequence according to SEQ ID NOs: 7, 8, 9, or 74.
178. The polyribonucleotide of any one of items 170 to 174, wherein the HSV glycoprotein is HSV-2 gE.
179. The polyribonucleotide of item 178, wherein the HSV-2 gE is or comprises an amino acid sequence that is at least 80%, 85%, 90%, 91%, 92%, 93%, 94%, 95%, 96%, 97%, 98%, or 99% identical to SEQ ID NOs: 66, 67, 68, or 69.
180. The polyribonucleotide of any one of items 178 to 179, wherein the HSV-2 gE consists or comprises an amino acid sequence according to SEQ ID NOs: 66, 67, 68, or 69.
181. The polyribonucleotide of any one of items 178 to 180, wherein the sequence is at least 80% identical to SEQ ID NOs: 80, 81, 82, 83, or 84.
182. The polyribonucleotide of any one of items 170 to 174, wherein the HSV glycoprotein is HSV-2 gH.
183. The polyribonucleotide of item 182, wherein the HSV-2 gH is or comprises an amino acid sequence that is at least 80%, 85%, 90%, 91%, 92%, 93%, 94%, 95%, 96%, 97%, 98%, or 99% identical to SEQ ID NO: 70, 71, 72, or 74.
184. The polyribonucleotide of any one of items 182 to 183, wherein the HSV-2 gH consists or comprises an amino acid sequence according to SEQ ID NO: 70, 71, 72, or 74.
185. The polyribonucleotide of any one of items 170 to 174, wherein the HSV glycoprotein is HSV-2 gI.
186. The polyribonucleotide of item 185, wherein the HSV-2 gI is or comprises an amino acid sequence that is at least 80%, 85%, 90%, 91%, 92%, 93%, 94%, 95%, 96%, 97%, 98%, or 99% identical to SEQ ID NO: 62, 63, 64, or 65.
187. The polyribonucleotide of any one of items 185 to 186, wherein the HSV-2 gI consists or comprises an amino acid sequence according to SEQ ID NO: 62, 63, 64, or 65.
188. The polyribonucleotide of any one of items 185 to 187, wherein the sequence is at least 80% identical to SEQ ID NO: 75, 76, 77, 78, or 79.
189. The polyribonucleotide of any one of items 170 to 174, wherein the HSV glycoprotein is HSV-2 gL.
190. The polyribonucleotide of item 190, wherein the HSV-2 gL is or comprises an amino acid sequence that is at least 80%, 85%, 90%, 91%, 92%, 93%, 94%, 95%, 96%, 97%, 98%, or 99% identical to SEQ ID NOs: 58, 59, 60, or 61.
191. The polyribonucleotide of any one of items 190 to 191, wherein the HSV-2 gL consists or comprises an amino acid sequence according to SEQ ID NOs: 58, 59, 60, or 61.
192. The polyribonucleotide of any one of items 1 to 192, wherein the polypeptide comprises a secretory signal.
193. The polyribonucleotide of item 192, wherein the secretory signal comprises or consists of a viral secretory signal.
194. The polyribonucleotide of item 193, wherein the viral secretory signal comprises or consists of an HSV secretory signal.
195. The polyribonucleotide of any one of items 192 to 194, wherein the secretory signal is a heterologous secretory signal.
196. The polyribonucleotide of any one of items 194 to 195, wherein the HSV secretory signal comprises or consists of an HSV-1 or HSV-2 secretory signal.
197. The polyribonucleotide of any one of items 194 to 196, wherein the HSV secretory signal is selected from:
   a) a gD2 secretion signal;
   b) a gD1 secretion signal;
   c) a gB1 secretion signal;
   d) a gI2 secretion signal;
   e) a gE2 secretion signal;
   f) a gC2 secretion signal:
   g) an Eboz secretion signal;
   h) an IL2 secretion signal; and
   i) an HLA-DR secretion signal.
198. The polyribonucleotide of any one of item 194 to 197, wherein the HSV secretory signal comprises or consists of an HSV gD secretory signal.
199. The polyribonucleotide of item 198, wherein the HSV gD secretory signal comprises an amino acid sequence that is at least 80% identical to SEQ ID NO: 87.
200. The polyribonucleotide of item 198, wherein the HSV gD secretory signal consists of an amino acid sequence according to SEQ ID NO: 88.
201. The polyribonucleotide of item 198, wherein the HSV gD secretory signal consists of an amino acid sequence according to SEQ ID NO: 110.
202. The polyribonucleotide of item 198, wherein the HSV gD secretory signal consists of an amino acid sequence according to SEQ ID NO: 111.
203. The polyribonucleotide of any one of items 198 to 202, wherein the secretory signal is located at the N-terminus of the polypeptide.
204. The polyribonucleotide of any one of item 194 to 197, wherein the HSV secretory signal comprises or consists of an HSV-2 glycoprotein I (gI) secretory signal.
205. The polyribonucleotide of item 204, wherein the HSV-2 gI secretory signal comprises an amino acid sequence that is at least 80% identical to SEQ ID NO: 107.
206. The polyribonucleotide of item 204, wherein the HSV-2 gI secretory signal comprises an amino acid sequence that is at least 80% identical to SEQ ID NO: 108.
207. The polyribonucleotide of any one of items 124 to 206, wherein the polypeptide comprises a transmembrane region.
208. The polyribonucleotide of item 207, wherein the transmembrane region comprises or consists of a viral transmembrane region.
209. The polyribonucleotide of any one of items 207 to 208, wherein the transmembrane region comprises or consists of an HSV transmembrane region.
210. The polyribonucleotide of any one of items 207 to 209, wherein the HSV transmembrane region comprises or consists of an HSV-1 or HSV-2 transmembrane region.
211. The polyribonucleotide of any one of items 207 to 210, wherein the HSV transmembrane region comprises or consists of an HSV gD transmembrane region.
212. The polyribonucleotide of item 211, wherein the HSV gD transmembrane region consists of an amino acid sequence according to SEQ ID NO: 160.
213. The polyribonucleotide of any one of items 124 to 206, wherein the polypeptide does not comprise a transmembrane region.
214. The polyribonucleotide of any one of items 124 to 213, wherein the polypeptide comprises a multimerization domain.
215. The polyribonucleotide of any one of items 124 to 214, wherein the polypeptide comprises one or more linkers.
216. The polyribonucleotide of item 215, wherein the one or more linkers comprise one or more glycine (G) residues and/or one or more serine (S) residues.
217. The polyribonucleotide of any one of items 215 to 216, wherein the one or more linkers comprise or consist of an amino acid sequence according to SEQ ID NO: 163.
218. The polyribonucleotide of any one of items 215 to 217, wherein the one or more linkers comprise or consist of an amino acid sequence according to SEQ ID NO: 165.
219. The polyribonucleotide of any one of items 215 to 217, wherein the one or more linkers comprise or consist of an amino acid sequence according to SEQ ID NO: 168.
220. The polyribonucleotide of item any one of items 215 to 217, wherein the one or more linkers comprise or consist of an amino acid sequence according to SEQ ID NO: 217.
221. The polyribonucleotide of any one of items 124 to 220, wherein the polyribonucleotide is an isolated polyribonucleotide.
222. The polyribonucleotide of any one of items 124 to 221, wherein the polyribonucleotide is an engineered polyribonucleotide.
223. The polyribonucleotide of any one of items 124 to 222, wherein the polyribonucleotide is a codon-optimized polyribonucleotide.
224. An RNA construct comprising in 5′ to 3′ order:
   (i) a 5′ UTR;
   (ii) a polyribonucleotide of any one of items 124 to 223;
   (iv) a 3′ UTR; and
   (v) a polyA tail sequence.
225. The RNA construct of item 224, wherein:
   (i) the 5′ UTR comprises or consists of a modified human alpha-globin 5′-UTR;
   (ii) the 3′ UTR that comprises or consists of a first sequence from the amino terminal enhancer of split (AES) messenger RNA and a second sequence from the mitochondrial encoded 12S ribosomal RNA; or
   (iii) both.
226. The RNA construct of item 224 or 225, wherein the 5′ UTR comprises or consists of a ribonucleic acid sequence according to SEQ ID NO: 208.
227. The RNA construct of item 224 or 225, wherein the 5′ UTR comprises or consists of a ribonucleic acid sequence according to SEQ ID NO: 209.
228. The RNA construct of any one of items 224 to 227, wherein the 3′ UTR comprises or consists ribonucleic acid sequence according to SEQ ID NO: 215.
229. The RNA construct of any one of items 224 to 227, wherein the 3′ UTR comprises or consists of a ribonucleic acid sequence according to SEQ ID NO: 216.
230. The RNA construct of any one of items 224 to 229, wherein the polyA tail sequence is a split polyA tail sequence.
231. The RNA construct of item 230, wherein the split polyA tail sequence consists of a ribonucleic acid sequence selected from SEQ ID NOs: 210, 212, or 213.
232. The RNA construct of any one of items 224 to 231, further comprising a 5′ cap.
233. The RNA construct of item 232, further comprising a cap proximal sequence comprising positions +1, +2, +3, +4, and +5 of the polyribonucleotide.
234. The RNA construct of item 232 or 233, wherein the 5′ cap comprises or consists of m7(3′OMeG)(5′)ppp(5′)(2′OMeA₁)pG₂, wherein A₁ is position +1 of the polyribonucleotide, and G₂ is position +2 of the polyribonucleotide.
235. The RNA construct of item 233 or 234, wherein the cap proximal sequence comprises A₁ and G₂ of the Cap1 structure, and a sequence comprising: A₃A₄U₅ (SEQ ID NO: 207) at positions +3, +4 and +5 respectively of the polyribonucleotide.
236. The RNA construct of any one of items 224 to 235, wherein the polyribonucleotide includes modified uridines in place of all uridines, optionally wherein modified uridines are each N1-methyl-pseudouridine.
237. A composition comprising one or more polyribonucleotides of any one of items 124 to 223.
238. A composition comprising one or more RNA constructs of any one of items 224 to 236.
239. The composition of item 237 or 238, wherein the composition further comprises lipid nanoparticles, polyplexes (PLX), lipidated polyplexes (LPLX), or liposomes,
   wherein the one or more polyribonucleotides are fully or partially encapsulated within the lipid nanoparticles, polyplexes (PLX), lipidated polyplexes (LPLX), or liposomes.
240. The composition of any one of items 237 to 239, wherein the composition further comprises lipid nanoparticles,
   wherein the one or more polyribonucleotides are encapsulated within the lipid nanoparticles.
241. A pharmaceutical composition comprising the composition of any one of items 237 to 240 and at least one pharmaceutically acceptable excipient.
242. The pharmaceutical composition of item 241, wherein the pharmaceutical comprises a cryoprotectant, optionally wherein the cryoprotectant is sucrose.
243. The pharmaceutical composition of item 241 or 242, wherein the pharmaceutical comprises an aqueous buffered solution, optionally wherein the aqueous buffered solution comprises one or more of Tris base, Tris HCl, NaCl, KCl, Na₂HPO₄, and KH₂PO₄.
244. A combination comprising:
   a first polyribonucleotide according to any one of items 124 to 223; and
   a second polyribonucleotide according to any one of items 124 to 223,
   wherein the first polyribonucleotide and the second polyribonucleotide are different.
245. A combination comprising:
   a first pharmaceutical composition comprising a first polyribonucleotide, wherein the first polyribonucleotide is a polyribonucleotide according to any one of items 124 to 223; and
   a second pharmaceutical composition comprising a second polyribonucleotide, wherein the second polyribonucleotide is a polyribonucleotide according to any one of items 124 to 223,
   wherein the first polyribonucleotide and the second polyribonucleotide are different.
246. A combination comprising:
   a first polyribonucleotide according to any one of items 169 to 191; and
   a second polyribonucleotide according to any one of items 138 to 168.
247. A combination comprising:
   a first polyribonucleotide according to any one of items 169 to 191; and
   a second polyribonucleotide encoding a second polypeptide, wherein the second polypeptide comprises one or more HSV RL2 polypeptides or antigenic fragments thereof, one or more HSV RS1 polypeptides or antigenic fragments thereof, and one or more HSV UL54 polypeptides or antigenic fragments thereof.
248. The combination of item 247, wherein the second polypeptide comprises, in N-terminus to C-terminus order, an HSV-1 gD secretory signal, an RL2 polypeptide or antigenic fragment thereof, a linker, an RL2 polypeptide or antigenic fragment thereof, a linker, an RS1 polypeptide or antigenic fragment thereof, a linker, a UL54 polypeptide or antigenic fragment thereof, a linker, and a MITD.
249. The combination of item 247 or 248, wherein the second polypeptide comprises or consists of an amino acid sequence according to SEQ ID NO: 197.
250. The combination of item 247, wherein the second polypeptide comprises, in N-terminus to C-terminus order, an HSV-1 gD secretory signal, an UL54 polypeptide or antigenic fragment thereof, a linker, an RS1 polypeptide or antigenic fragment thereof, a linker, an RL2 polypeptide or antigenic fragment thereof, a linker, a RL2 polypeptide or antigenic fragment thereof, a linker, and a MITD.
251. The combination of item 247 or 250, wherein the second polypeptide comprises or consists of an amino acid sequence according to SEQ ID NO: 201.
252. The combination of item 247, wherein the second polypeptide comprises, in N-terminus to C-terminus order, an HSV-2 gD secretory signal, an RL2 polypeptide or antigenic fragment thereof, a linker, an RL2 polypeptide or antigenic fragment thereof, a linker, an RS1 polypeptide or antigenic fragment thereof, a linker, a UL54 polypeptide or antigenic fragment thereof, a linker, and a MITD.
253. The combination of item 247 or 252, wherein the second polypeptide comprises or consists of an amino acid sequence according to SEQ ID NO: 205.
254. A combination comprising:
   a first polyribonucleotide according to any one of items 169 to 191; and
   a second polyribonucleotide encoding a second polypeptide, wherein the second polypeptide comprises one or more HSV UL29 polypeptides or antigenic fragments thereof, one or more HSV UL39 polypeptides or antigenic fragments thereof, one or more HSV UL49 polypeptides or antigenic fragments thereof, and one or more HSV UL9 polypeptides or antigenic fragments thereof.
255. The combination of item 254, wherein the second polypeptide comprises, in N-terminus to C-terminus order, an HSV-1 gD secretory signal, an UL29 polypeptide or antigenic fragment thereof, a linker, an UL39 polypeptide or antigenic fragment thereof, a linker, an UL49 polypeptide or antigenic fragment thereof, a linker, a UL9 polypeptide or antigenic fragment thereof, a linker, and a MITD.
256. The combination of item 254 or 255, wherein the second polypeptide comprises or consists of an amino acid sequence according to SEQ ID NO: 198.
257. The combination of item 254, wherein the second polypeptide comprises, in N-terminus to C-terminus order, an HSV-1 gD secretory signal, an UL9 polypeptide or antigenic fragment thereof, a linker, an UL49 polypeptide or antigenic fragment thereof, a linker, an UL39 polypeptide or antigenic fragment thereof, a linker, a UL29 polypeptide or antigenic fragment thereof, a linker, and a MITD.
258. The combination of item 254 or 257, wherein the second polypeptide comprises or consists of an amino acid sequence according to SEQ ID NO: 202.
259. A combination comprising:
   a first polyribonucleotide according to any one of items 169 to 191; and
   a second polyribonucleotide encoding a second polypeptide, wherein the second polypeptide comprises one or more HSV UL30 polypeptides or antigenic fragments thereof, one or more HSV UL40 polypeptides or antigenic fragments thereof, one or more HSV UL5 polypeptides or antigenic fragments thereof, and one or more HSV UL52 polypeptides or antigenic fragments thereof.
260. The combination of item 259, wherein the second polypeptide comprises, in N-terminus to C-terminus order, an HSV-1 gD secretory signal, an UL30 polypeptide or antigenic fragment thereof, a linker, an UL30 polypeptide or antigenic fragment thereof, a linker, an UL40 polypeptide or antigenic fragment thereof, a linker, a UL5 polypeptide or antigenic fragment thereof, a linker, a UL5 polypeptide or antigenic fragment thereof, a linker, a UL52 polypeptide or antigenic fragment thereof, a linker, and a MITD.
261. The combination of item 259 or 260, wherein the second polypeptide comprises or consists of an amino acid sequence according to SEQ ID NO: 199.
262. The combination of item 259, wherein the second polypeptide comprises, in N-terminus to C-terminus order, an HSV-1 gD secretory signal, an UL52 polypeptide or antigenic fragment thereof, a linker, an UL5 polypeptide or antigenic fragment thereof, a linker, an UL5 polypeptide or antigenic fragment thereof, a linker, a UL40 polypeptide or antigenic fragment thereof, a linker, a UL30 polypeptide or antigenic fragment thereof, a linker, a UL30 polypeptide or antigenic fragment thereof, a linker, and a MITD.
263. The combination of item 259 or 262, wherein the second polypeptide comprises or consists of an amino acid sequence according to SEQ ID NO: 203.
264. A combination comprising:
   a first polyribonucleotide according to any one of items 169 to 191; and
   a second polyribonucleotide encoding a second polypeptide, wherein the second polypeptide comprises one or more HSV UL1 polypeptides or antigenic fragments thereof, one or more HSV UL19 polypeptides or antigenic fragments thereof, one or more HSV UL21 polypeptides or antigenic fragments thereof, one or more HSV UL27 polypeptides or antigenic fragments thereof, one or more HSV UL46 polypeptides or antigenic fragments thereof, one or more HSV UL47 polypeptides or antigenic fragments thereof, one or more UL48 polypeptides or antigenic fragments thereof, and one or more HSV UL25 polypeptides or antigenic fragments thereof.
265. The combination of item 264, wherein the second polypeptide comprises, in N-terminus to C-terminus order, an HSV-1 gD secretory signal, an HSV UL1 polypeptide or antigenic fragment thereof, a linker, an HSV UL19 polypeptide or antigenic fragment thereof, a linker, an HSV UL21 polypeptide or antigenic fragment thereof, a linker, a HSV UL27 polypeptide or antigenic fragment thereof, a linker, a HSV UL27 polypeptide or antigenic fragment thereof, a linker, a HSV UL46 polypeptide or antigenic fragment thereof, a linker, a HSV UL47 polypeptide or antigenic fragment thereof, a linker, a HSV UL25 polypeptide or antigenic fragment thereof, a linker, a HSV UL48 polypeptide or antigenic fragment thereof, a linker, and a MITD.
266. The combination of item 264 or 265, wherein the second polypeptide comprises or consists of an amino acid sequence according to SEQ ID NO: 200.
267. The combination of item 264, wherein the second polypeptide comprises, in N-terminus to C-terminus order, an HSV-1 gD secretory signal, an HSV UL48 polypeptide or antigenic fragment thereof, a linker, an HSV UL25 polypeptide or antigenic fragment thereof, a linker, an HSV UL47 polypeptide or antigenic fragment thereof, a linker, a HSV UL46 polypeptide or antigenic fragment thereof, a linker, a HSV UL27 polypeptide or antigenic fragment thereof, a linker, a HSV UL27 polypeptide or antigenic fragment thereof, a linker, a HSV UL21 polypeptide or antigenic fragment thereof, a linker, a HSV UL19 polypeptide or antigenic fragment thereof, a linker, a HSV UL1 polypeptide or antigenic fragment thereof, a linker, and a MITD.
268. The combination of item 264 or 267, wherein the second polypeptide comprises or consists of an amino acid sequence according to SEQ ID NO: 204.
269. The combination of any one of items 264 to 268, wherein the second polypeptide is an HSV gB.
270. The combination of one of items 264 to 269, wherein the second polypeptide consists or comprises an amino acid sequence according to SEQ ID NOs: 7, 8, 9, or 74.
271. A combination comprising:
   a first pharmaceutical composition comprising a first polyribonucleotide, wherein the first polyribonucleotide is a polyribonucleotide according to any one of items 169 to 191; and
   a second pharmaceutical composition comprising a second polyribonucleotide, wherein the second polyribonucleotide is a polyribonucleotide according to any one of items 138 to 168.
272. A combination comprising:
   a first pharmaceutical composition comprising a first polyribonucleotide, wherein the first polyribonucleotide is a polyribonucleotide according to any one of items 169 to 191; and
   a second pharmaceutical composition comprising a second polyribonucleotide, wherein the second polyribonucleotide comprises one or more HSV RL2 polypeptides or antigenic fragments thereof, one or more HSV RS1 polypeptides or antigenic fragments thereof, and one or more HSV UL54 polypeptides or antigenic fragments thereof.
273. The combination of item 272, wherein the second polypeptide comprises, in N-terminus to C-terminus order, an HSV-1 gD secretory signal, an RL2 polypeptide or antigenic fragment thereof, a linker, an RL2 polypeptide or antigenic fragment thereof, a linker, an RS1 polypeptide or antigenic fragment thereof, a linker, a UL54 polypeptide or antigenic fragment thereof, a linker, and a MITD.
274. The combination of item 272 or 273, wherein the second polypeptide comprises or consists of an amino acid sequence according to SEQ ID NO: 197.
275. The combination of item 272, wherein the second polypeptide comprises, in N-terminus to C-terminus order, an HSV-1 gD secretory signal, an UL54 polypeptide or antigenic fragment thereof, a linker, an RS1 polypeptide or antigenic fragment thereof, a linker, an RL2 polypeptide or antigenic fragment thereof, a linker, a RL2 polypeptide or antigenic fragment thereof, a linker, and a MITD.
276. The combination of item 272 or 275, wherein the second polypeptide comprises or consists of an amino acid sequence according to SEQ ID NO: 201.
277. The combination of item 272, wherein the second polypeptide comprises, in N-terminus to C-terminus order, an HSV-2 gD secretory signal, an RL2 polypeptide or antigenic fragment thereof, a linker, an RL2 polypeptide or antigenic fragment thereof, a linker, an RS1 polypeptide or antigenic fragment thereof, a linker, a UL54 polypeptide or antigenic fragment thereof, a linker, and a MITD.
278. The combination of item 272 or 277, wherein the second polypeptide comprises or consists of an amino acid sequence according to SEQ ID NO: 205.
279. A combination comprising:
   a first pharmaceutical composition comprising a first polyribonucleotide, wherein the first polyribonucleotide is a polyribonucleotide according to any one of items 169 to 191; and
   a second pharmaceutical composition comprising a second polyribonucleotide, wherein the second polypeptide comprises one or more HSV UL29 polypeptides or antigenic fragments thereof, one or more HSV UL39 polypeptides or antigenic fragments thereof, one or more HSV UL49 polypeptides or antigenic fragments thereof, and one or more HSV UL9 polypeptides or antigenic fragments thereof.
280. The combination of item 279, wherein the second polypeptide comprises, in N-terminus to C-terminus order, an HSV-1 gD secretory signal, an UL29 polypeptide or antigenic fragment thereof, a linker, an UL39 polypeptide or antigenic fragment thereof, a linker, an UL49 polypeptide or antigenic fragment thereof, a linker, a UL9 polypeptide or antigenic fragment thereof, a linker, and a MITD.
281. The combination of item 279 or 280, wherein the second polypeptide comprises or consists of an amino acid sequence according to SEQ ID NO: 198.
282. The combination of item 279, wherein the second polypeptide comprises, in N-terminus to C-terminus order, an HSV-1 gD secretory signal, an UL9 polypeptide or antigenic fragment thereof, a linker, an UL49 polypeptide or antigenic fragment thereof, a linker, an UL39 polypeptide or antigenic fragment thereof, a linker, a UL29 polypeptide or antigenic fragment thereof, a linker, and a MITD.
283. The combination of item 279 or 282, wherein the second polypeptide comprises or consists of an amino acid sequence according to SEQ ID NO: 202.
284. A combination comprising:
   a first pharmaceutical composition comprising a first polyribonucleotide, wherein the first polyribonucleotide is a polyribonucleotide according to any one of items 169 to 191; and
   a second pharmaceutical composition comprising a second polyribonucleotide, wherein the second polypeptide comprises one or more HSV UL30 polypeptides or antigenic fragments thereof, one or more HSV UL40 polypeptides or antigenic fragments thereof, one or more HSV UL5 polypeptides or antigenic fragments thereof, and one or more HSV UL52 polypeptides or antigenic fragments thereof.
285. The combination of item 284, wherein the second polypeptide comprises, in N-terminus to C-terminus order, an HSV-1 gD secretory signal, an UL30 polypeptide or antigenic fragment thereof, a linker, an UL30 polypeptide or antigenic fragment thereof, a linker, an UL40 polypeptide or antigenic fragment thereof, a linker, a UL5 polypeptide or antigenic fragment thereof, a linker, a UL5 polypeptide or antigenic fragment thereof, a linker, a UL52 polypeptide or antigenic fragment thereof, a linker, and a MITD.
286. The combination of item 284 or 285, wherein the second polypeptide comprises or consists of an amino acid sequence according to SEQ ID NO: 199.
287. The combination of item 284, wherein the second polypeptide comprises, in N-terminus to C-terminus order, an HSV-1 gD secretory signal, an UL52 polypeptide or antigenic fragment thereof, a linker, an UL5 polypeptide or antigenic fragment thereof, a linker, an UL5 polypeptide or antigenic fragment thereof, a linker, a UL40 polypeptide or antigenic fragment thereof, a linker, a UL30 polypeptide or antigenic fragment thereof, a linker, a UL30 polypeptide or antigenic fragment thereof, a linker, and a MITD.
288. The combination of item 284 or 287, wherein the second polypeptide comprises or consists of an amino acid sequence according to SEQ ID NO: 203.
289. A combination comprising:
   a first pharmaceutical composition comprising a first polyribonucleotide, wherein the first polyribonucleotide is a polyribonucleotide according to any one of items 169 to 191; and
   a second pharmaceutical composition comprising a second polyribonucleotide, wherein the second polypeptide comprises one or more HSV UL1 polypeptides or antigenic fragments thereof, one or more HSV UL19 polypeptides or antigenic fragments thereof, one or more HSV UL21 polypeptides or antigenic fragments thereof, one or more HSV UL27 polypeptides or antigenic fragments thereof, one or more HSV UL46 polypeptides or antigenic fragments thereof, one or more HSV UL47 polypeptides or antigenic fragments thereof, one or more UL48 polypeptides or antigenic fragments thereof, and one or more HSV UL25 polypeptides or antigenic fragments thereof.
290. The combination of item 289, wherein the second polypeptide comprises, in N-terminus to C-terminus order, an HSV-1 gD secretory signal, an HSV UL1 polypeptide or antigenic fragment thereof, a linker, an HSV UL19 polypeptide or antigenic fragment thereof, a linker, an HSV UL21 polypeptide or antigenic fragment thereof, a linker, a HSV UL27 polypeptide or antigenic fragment thereof, a linker, a HSV UL27 polypeptide or antigenic fragment thereof, a linker, a HSV UL46 polypeptide or antigenic fragment thereof, a linker, a HSV UL47 polypeptide or antigenic fragment thereof, a linker, a HSV UL25 polypeptide or antigenic fragment thereof, a linker, a HSV UL48 polypeptide or antigenic fragment thereof, a linker, and a MITD.
291. The combination of item 289 or 290, wherein the second polypeptide comprises or consists of an amino acid sequence according to SEQ ID NO: 200.
292. The combination of item 289, wherein the second polypeptide comprises, in N-terminus to C-terminus order, an HSV-1 gD secretory signal, an HSV UL48 polypeptide or antigenic fragment thereof, a linker, an HSV UL25 polypeptide or antigenic fragment thereof, a linker, an HSV UL47 polypeptide or antigenic fragment thereof, a linker, a HSV UL46 polypeptide or antigenic fragment thereof, a linker, a HSV UL27 polypeptide or antigenic fragment thereof, a linker, a HSV UL27 polypeptide or antigenic fragment thereof, a linker, a HSV UL21 polypeptide or antigenic fragment thereof, a linker, a HSV UL19 polypeptide or antigenic fragment thereof, a linker, a HSV UL1 polypeptide or antigenic fragment thereof, a linker, and a MITD.
293. The combination of item 289 or 292, wherein the second polypeptide comprises or consists of an amino acid sequence according to SEQ ID NO: 204.
294. The combination of any one of items 289 to 293, wherein the second polypeptide is an HSV gB.
295. The combination of one of items 289 to 294, wherein the second polypeptide consists or comprises an amino acid sequence according to SEQ ID NOs: 7, 8, 9, or 74.
296. A method comprising administering a polyribonucleotide according to any one of items 124 to 223, or an RNA construct according to any one of items 224 to 236, to a subject.
297. A method comprising administering a composition according to any one of items 237 to 240, to a subject.
298. A method comprising administering one or more doses of the composition of any one of items 237 to 240 or the pharmaceutical composition of any one of items 241 to 243, to a subject.
299. A method comprising administering a combination of any one of items 244 to 294, to a subject.
300. The pharmaceutical composition of any one of items 241 to 243, for use in the treatment of an HSV infection comprising administering one or more doses of the pharmaceutical composition to a subject.
301. The pharmaceutical composition of any one of items 241 to 243, for use in the prevention of an HSV infection comprising administering one or more doses of the pharmaceutical composition to a subject.
302. The method of item 299 or the pharmaceutical composition for use of item 300 or 301, comprising administering two or more doses of the pharmaceutical composition to a subject.
303. The method of item 299 or the pharmaceutical composition for use of item 300 or 301, comprising administering three or more doses of the pharmaceutical composition to a subject.
304. A method comprising administering a combination of any one of items 271 to 294 to a subject.
305. The method of item 304, wherein the first pharmaceutical composition and the second pharmaceutical composition are administered on the same day.
306. The method of item 304 and 305, wherein the first pharmaceutical composition and the second pharmaceutical composition are administered on different days.
307. The method of any one of items 304 to 306, wherein the first pharmaceutical composition and the second pharmaceutical composition are administered to the subject at different locations on the subject's body.
308. The method of any one of items 304 to 307, wherein the method is a method of treating an HSV infection.
309. The method of any one of items 304 to 308, wherein the method is a method of preventing an HSV infection.
310. The method of any one of items 304 to 309, wherein the subject has or is at risk of developing an HSV infection.
311. The method of any one of items 304 to 310, wherein the subject is a human.
312. The method of any one of items 304 to 311, wherein administration induces an anti-HSV immune response in the subject.
313. The method of item 312, wherein the anti-HSV immune response in the subject comprises an adaptive immune response.
314. The method of item 312 or 313, wherein the anti-HSV immune response in the subject comprises a T-cell response.
315. The method of item 314, wherein the T-cell response is or comprises a CD4+ T cell response.
316. The method of item 314, wherein the T-cell response is or comprises a CD8+ T cell response.
317. The method of item 312, wherein the anti-HSV immune system response comprises a B-cell response.
318. The method of any one of items 312 to 317, wherein the anti-HSV immune system response comprises the production of antibodies directed against the one or more HSV antigens or antigenic fragments thereof have at least 80% sequence identity with one or more sequences selected from SEQ ID NOs: 1-74 or an antigenic fragment thereof.
319. Use of the pharmaceutical composition of any one of items 241 to 243, in the treatment of a herpes simplex virus infection.
320. Use of the pharmaceutical composition of any one of items 241 to 243 in the prevention of a herpes simplex virus infection.
321. Use of the pharmaceutical composition of any one of items 241 to 243, in inducing an anti-herpes simplex immune virus response in a subject.
322. A polypeptide encoded by a polyribonucleotide of any one of items 124 to 223.
323. A polypeptide encoded by an RNA construct of any one of items 224 to 236.
324. A host cell comprising a polyribonucleotide of any one of items 124 to 223.
325. A host cell comprising an RNA construct of any one of items 224 to 236.
326. A host cell comprising a polypeptide of item 322 or 323.

### EQUIVALENTS

Those skilled in the art will recognize, or be able to ascertain using no more than routine experimentation, many equivalents to the specific embodiments of technologies described herein. The scope of the present disclosure is not intended to be limited to the above Description, but rather is as set forth in the following claims:

1. A polyribonucleotide encoding a polypeptide, wherein the polypeptide comprises two or more herpes simplex virus (HSV) antigens or antigenic fragments thereof, and wherein the two or more HSV antigens or antigenic fragments thereof comprise:
(i) one or more HSV RS1 polypeptides or antigenic fragments thereof,
(ii) one or more HSV RL2 polypeptides or antigenic fragments thereof,
(iii) one or more HSV UL1 polypeptides or antigenic fragments thereof,
(iv) one or more HSV UL5 polypeptides or antigenic fragments thereof,
(v) one or more HSV UL9 polypeptides or antigenic fragments thereof,
(vi) one or more HSV UL19 polypeptides or antigenic fragments thereof,
(vii) one or more HSV UL21 polypeptides or antigenic fragments thereof,
(viii) one or more HSV UL25 polypeptides or antigenic fragments thereof,
(ix) one or more HSV UL27 polypeptides or antigenic fragments thereof,
(x) one or more HSV UL29 polypeptides or antigenic fragments thereof,
(xi) one or more HSV UL30 polypeptides or antigenic fragments thereof,
(xii) one or more HSV UL39 polypeptides or antigenic fragments thereof,
(xiii) one or more HSV UL40 polypeptides or antigenic fragments thereof,
(xiv) one or more HSV UL46 polypeptides or antigenic fragments thereof,
(xv) one or more HSV UL47 polypeptides or antigenic fragments thereof,
(xvi) one or more HSV UL48 polypeptides or antigenic fragments thereof,
(xvii) one or more HSV UL49 polypeptides or antigenic fragments thereof,
(xviii) one or more HSV UL52 polypeptides or antigenic fragments thereof, and/or
(xix) one or more HSV UL54 polypeptides or antigenic fragments thereof.

2-10. (canceled)

11. The polyribonucleotide of claim 1, wherein the polypeptide comprises:
(a) one or more HSV UL29 polypeptides or antigenic fragments thereof, one or more HSV UL39 polypeptides or antigenic fragments thereof, one or more HSV UL49 polypeptides or antigenic fragments thereof, and one or more HSV UL9 polypeptides or antigenic fragments thereof, or
(b) one or more HSV UL30 polypeptides or antigenic fragments thereof, one or more HSV UL40 polypeptides or antigenic fragments thereof, one or more HSV UL5 polypeptides or antigenic fragments thereof, and one or more HSV UL52 polypeptides or antigenic fragments thereof, or
(c) one or more HSV UL1 polypeptides or antigenic fragments thereof, one or more HSV UL19 polypeptides or antigenic fragments thereof, one or more HSV UL21 polypeptides or antigenic fragments thereof, one or more HSV UL27 polypeptides or antigenic fragments thereof, one or more HSV UL46 polypeptides or antigenic fragments thereof, one or more HSV UL47 polypeptides or antigenic fragments thereof, one or more UL48 polypeptides or antigenic fragments thereof, and one or more HSV UL25 polypeptides or antigenic fragments thereof.

12-30. (canceled)

31. The polyribonucleotide of claim 1, wherein the polyribonucleotide is a codon-optimized polyribonucleotide.

32. (canceled)

33. A combination comprising:
a first pharmaceutical composition comprising a first polyribonucleotide, wherein the first polyribonucleotide is a polyribonucleotide according to claim 1, and
a second pharmaceutical composition comprising a second polyribonucleotide, wherein the polyribonucleotide encodes a second polypeptide, wherein the second polypeptide comprises one or more herpes simplex virus (HSV) antigens or antigenic fragments thereof,
wherein the first polyribonucleotide and the second polyribonucleotide are different.

34. The combination of claim 33, wherein the one or more HSV antigens or antigenic fragments thereof of the second polypeptide comprise one or more HSV glycoprotein polypeptides or antigenic fragments thereof.

35. The combination of claim 34, wherein the one or more HSV glycoprotein polypeptides or antigenic fragments thereof comprise an HSV glycoprotein B (gB) polypeptide or antigenic fragment thereof, an HSV glycoprotein E (gE) polypeptide or antigenic fragment thereof, an HSV glycoprotein G (gG) polypeptide or antigenic fragment thereof, an HSV glycoprotein H (gH) polypeptide or antigenic fragment thereof, an HSV glycoprotein I (gI) polypeptide or antigenic fragment thereof, an HSV glycoprotein L (gL) polypeptide or antigenic fragment thereof, an HSV glycoprotein D (gD) polypeptide or antigenic fragment thereof, and/or an HSV glycoprotein C (gC) polypeptide or antigenic fragment thereof.

36-39. (canceled)

40. An RNA construct comprising in 5′ to 3′ order:
(i) a 5′ untranslated region (UTR),
(ii) a polyribonucleotide of claim 1,
(iv) a 3′ UTR, and
(v) a polyadenylate (polyA) tail sequence.

41. The RNA construct of claim 40, further comprising a 5′ cap.

42. A composition comprising:
(i) one or more polyribonucleotides of claim 1, and
(ii) lipid nanoparticles, polyplexes (PLX), or liposomes,
wherein the one or more polyribonucleotides are fully or partially encapsulated within the lipid nanoparticles, polyplexes (PLX), or liposomes.

43. A pharmaceutical composition comprising one or more polyribonucleotides of claim 1 and at least one pharmaceutically acceptable excipient.

44. A method comprising administering a polyribonucleotide according to claim 1 to a subject.

45. A method comprising administering an RNA construct according to claim 40 to a subject.

46. A method comprising administering a pharmaceutical composition according to claim 43 to a subject.

47. A method comprising administering a combination according to claim 33 to a subject.

48. The polyribonucleotide of claim 1, wherein the two or more HSV antigens or antigenic fragments are different HSV antigens and/or antigenic fragments of two or more different HSV antigens.

49. The polyribonucleotide of claim 1, wherein the two or more HSV antigen or antigenic fragments comprise:
(i) one or more HSV RL2 polypeptides or antigenic fragments thereof,
(ii) one or more HSV UL54 polypeptides or antigenic fragments thereof,
(iii) one or more HSV UL5 polypeptides or antigenic fragments thereof,
(iv) one or more HSV UL40 polypeptides or antigenic fragments thereof,
(v) one or more HSV UL47 polypeptides or antigenic fragments thereof, and/or
(vi) one or more HSV UL46 polypeptides or antigenic fragments thereof.

50. The polyribonucleotide of claim 1, wherein the two or more HSV antigen or antigenic fragments comprise:
(i) one or more HSV RL2 polypeptides or antigenic fragments thereof,
(ii) one or more HSV UL54 polypeptides or antigenic fragments thereof,
(iii) one or more HSV UL47 polypeptides or antigenic fragments thereof,
(iv) one or more HSV UL46 polypeptides or antigenic fragments thereof, and/or
(v) one or more HSV UL21 polypeptides or antigenic fragments thereof.

51. The polyribonucleotide of claim 1, wherein the two or more HSV antigen or antigenic fragments comprise:
(i) one or more HSV UL29 polypeptides or antigenic fragments thereof,
(ii) one or more HSV UL39 polypeptides or antigenic fragments thereof,
(iii) one or more HSV UL9 polypeptides or antigenic fragments thereof,
(iv) one or more HSV UL5 polypeptides or antigenic fragments thereof,
(v) one or more HSV UL40 polypeptides or antigenic fragments thereof, and/or
(vi) one or more HSV UL30 polypeptides or antigenic fragments thereof.

52. The polyribonucleotide of claim 1, wherein the two or more HSV antigen or antigenic fragments comprise:
(a) one or more HSV RL2 polypeptides or antigenic fragments thereof, and one or more HSV UL54 polypeptides or antigenic fragments thereof, or
(b) one or more HSV UL5 polypeptides or antigenic fragments thereof, and one or more HSV UL40 polypeptides or antigenic fragments thereof, or
(c) one or more HSV UL47 polypeptides or antigenic fragments thereof, and one or more HSV UL46 polypeptides or antigenic fragments thereof, or
(d) one or more HSV RL2 polypeptides or antigenic fragments thereof, and one or more HSV UL47 polypeptides or antigenic fragments thereof.

53. The polyribonucleotide of claim 1, wherein the polypeptide comprises a secretory signal.

54. The polyribonucleotide of claim 53, wherein the secretory signal is an HSV secretory signal.

55. The polyribonucleotide of claim 54, wherein the HSV secretory signal is an HSV-1 gD secretory signal or an HSV-2 gD secretory signal.

56. The polyribonucleotide of claim 1, wherein the polypeptide comprises an MHC class I trafficking signal (MITD).

57. The polyribonucleotide of claim 1, wherein the polypeptide comprises one or more linkers, wherein the one or more linkers are located (a) between two antigens or antigenic fragments thereof, (b) between a secretory signal and an antigen or antigenic fragment thereof, (c) between an antigen or antigenic fragment thereof and an MITD.


SEQUENCE LISTING (241 sequences)

SEQ ID NO: 1 - protein, 224 aa, Human alphaherpesvirus 2
  source 1..224: note = HSV-2 strain HG52
MGFVCLFGLVVMGAWGAWGGSQATEYVLRSVIAKEVGDILRVPCMRTPADDVSWRYEAPSVIDYARIDGIFLRYHCPGLDTFLWDRHAQRAYLVNPFLFAAGFLEDLSHSVFPADTQETTTRRALYKEIRDALGSRKQAVSHAPVRAGCVNFDYSRTRRCVGRRDLRPANTTSTWEPPVSSDDEASSQSKPLATQPPVLALSNAPPRRVSPTRGRRRHTRLRRN

SEQ ID NO: 2 - protein, 224 aa, Human alphaherpesvirus 2; identical to SEQ ID NO: 1
  source 1..224: note = HSV-2 strain 333

SEQ ID NO: 3 - protein, 224 aa, Human alphaherpesvirus 2; identical to SEQ ID NO: 1
  source 1..224: note = HSV-2 strain MS

SEQ ID NO: 4 - protein, 532 aa, Human alphaherpesvirus 2
  source 1..532: note = HSV-2 strain HG52
MELSYATTLHHRDVVFYVTADRNRAYFVCGGSVYSVGRPRDSQPGEIAKFGLVVRGTGPKDRMVANYVRSELRQRGLRDVRPVGEDEVFLDSVCLLNPNVSSERDVINTNDVEVLDECLAEYCTSLRTSPGVLVTGVRVRARDRVIELFEHPAIVNISSRFAYTPSPYVFALAQAHLPRLPSSLEPLVSGLFDGIPAPRQPLDARDRRTDVVITGTRAPRPMAGTGAGGAGAKRATVSEFVQVKHIDRVVSPSVSSAPPPSAPDASLPPPGLQEAAPPGPPLRELWWVFYAGDRALEEPHAESGLTREEVRAVHGFREQAWKLFGSVGAPRAFLGAALALSPTQKLAVYYYLIHRERRMSPFPALVRLVGRYIQRHGLYVPAPDEPTLADAMNGLFRDALAAGTVAEQLLMFDLLPPKDVPVGSDARADSAALLRFVDSQRLTPGGSVSPEHVMYLGAFLGVLYAGHGRLAAATHTARLTGVTSLVLTVGDVDRMSAFDRGPAGAAGRTRTAGYLDALLTVCLARAQHGQSV

SEQ ID NO: 5 - protein, 532 aa, Human alphaherpesvirus 2; identical to SEQ ID NO: 4
  source 1..532: note = HSV-2 strain 333

SEQ ID NO: 6 - protein, 532 aa, Human alphaherpesvirus 2
  source 1..532: note = HSV-2 strain MS
MELSYATTLHHRDVVFYVTADRNRAYFVCGGSVYSVGRPRASQPGEIAKFGLVVRGTGPKDRMVANYVRSELRQRGLRDVRPVGEDEVFLDSVCLLNPNVSSERDVINTNDVEVLDECLAEYCTSLRTSPGVLVTGVRVRARDRVIELFEHPAIVNISSRFVYTPSPYVFALAQAHLPRLPSSLEPLVSGLFDGIPAPRQPLDARDRRTDVVITGTRAPRPMAGTGAGGAGAKRATVSEFVQVKHIDRVVSPSVSSAPPPSAPDASLPPPGLQEAAPPGPPLRELWWVFYAGDRALEEPHAESGLTREEVRAVHGFREQAWKLFGSVGAPRAFLGAALALSPTQKLAVYYYLIHRERRMSPFPALVRLVGRYIQRHGLYVPAPDEPTLADAMNGLFRDALAAGTVAEQLLMFDLLPPKDVPVGSDARADSAALLRFVDSQRLTPGGSVSPEHVMYLGAFLGVLYAGHGRLAAATHTARLTGVTSLVLTVGDVDRMSAFDRGPAGAAGRTRTAGYLDALLTVCLARAQHGQSV

SEQ ID NO: 7 - protein, 905 aa, Human alphaherpesvirus 2
  source 1..905: note = HSV-2 strain HG52
MRGGGLICALVVGALVAAVASAAPAAPAAPRASGGVAATVAANGGPASRPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINTKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTEYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPULIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 8 - protein, 904 aa, Human alphaherpesvirus 2
  source 1..904: note = HSV-2 strain 333
MRGGGLICALVVGALVAAVASAAPAAPAAPRASGGVAATVAANGGPASRPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 9 - protein, 901 aa, Human alphaherpesvirus 2
  source 1..901: note = HSV-2 strain MS
MRGGGLICALVVGALVAAVASAAPAAPRASGGVAATVAANGGPAPQPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKAQATSPTTRNLLTTPKFTVAWDWVPKRPAVCTMTKWQEVDEMLRAEYGGSFRFSSDAISTTFTTNLTEYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEARQMIRYMALVSAMERTEHKARKKGTSALLSSKVTNMVLRKRNKARYSPLHNEDEAGDEDEL

SEQ ID NO: 10 - protein, 1196 aa, Human alphaherpesvirus 2
  source 1..1196: note = HSV-2 strain HG52
MDTKPKTTTTVKVPPGPMGYVYGRACPAEGLELLSLLSARSGDADVAVAPLIVGLTVESGFEANVAAVVGSRTTGLGGTAVSLKLMPSHYSPSVYVFHGGRHLAPSTQAPNLTRLCERARPHFGFADYAPRPCDLKHETTGDALCERLGLDPDRALLYLVITEGFREAVCISNTFLHLGGMDKVTIGDAEVHRIPVYPLQMFMPDFSRVIADPFNCNHRSIGENFNYPLPFFNRPLARLLFEAVVGPAAVALRARNVDAVARAAAHLAFDENHEGAALPADITFTAFEASQGKPQRGARDAGNKGPAGGFEQRLASVMAGDAALALESIVSMAVFDEPPPDITTWPLLEGQETPAARAGAVGAYLARAAGLVGAMVFSTNSALHLTEVDDAGPADPKDHSKPSFYRFFLVPGTHVAANPQLDREGHVVPGYEGRPTAPLVGGTQEFAGEHLAMLCGFSPALLAKMLFYLERCDGGVIVGRQEMDVFRYVADSGQTDVPCNLCTFETRHACAHTTLMRLRARHPKFASAARGAIGVFGTMNSAYSDCDVLGNYAAFSALKRADGSENTRTIMQETYRAATERVMAELEALQYVDQAVPTALGRLETIIGNREALHTVVNNIKQLVDREVEQLMRNLIEGRNFKFRDGLAEANHAMSLSLDPYTCGPCPLLQLLARRSNLAVYQDLALSQCHGVFAGQSVEGRNFRNQFQPVLRRRVMDLFNNGFLSAKTLTVALSEGAAICAPSLTAGQTAPAESSFEGDVARVTLGFPKELRVKSRVLFAGASANASEAAKARVASLQSAYQKPDKRVDILLGPLGFLLKQFHAVIFPNGKPPGSNQPNPQWFWTALQRNQLPARLLSREDIETIAFIKRFSLDYGAINFINLAPNNVSELAMYYMANQILRYCDHSTYFINTLTAVIAGSRRPPSVQAAAAWAPQGGAGLEAGARALMDSLDAHPGAWTSMFASCNLLRPVMAARPMVVLGLSISKYYGMAGNDRVFQAGNWASLLGGKNACPLLIFDRTRKFVLACPRAGFVCAASSLGGGAHEHSLCEQLRGIIAEGGAAVASSVFVATVKSLGPRTQQLQIEDWLALLEDEYLSEEMMEFTTRALERGHGEWSTDAALEVAHEAEALVSQLGAAGEVFNFGDFGDEDDHAASFGGLAAAAGAAGVARKRAFHGDDPFGEGPPEKKDLTLDML

SEQ ID NO: 11 - protein, 1196 aa, Human alphaherpesvirus 2
  source 1..1196: note = HSV-2 strain 333
MDTKPKTTTTVKVPPGPMGYVYGRACPAEGLELLSLLSARSGDADVAVAPLIVGLTVESGFEANVAAVVGSRTTGLGGTAVSLKLMPSHYSPSVYVFHGGRHLAPSTQAPNLTRLCERARRHFGFSDYAPRPCDLKHETTGDALCERLGLDPDRALLYLVITEGFREAVCISNTFLHLGGMDKVTIGDAEVHRIPVYPLQMFMPDFSRVIADPFNCNHRSIGENFNYPLPFFNRPLARLLFEAVVGPAAVALRARNVDAVARAAAHLAFDENHEGAALPADITFTAFEASQGKPQRGARDAGNKGPAGGFEQRLASVMAGDAALALESIVSMAVFDEPPPDITTWPLLEGQETPAARAGAVGAYLARAAGLVGAMVFSTNSALHLTEVDDAGPADPKDHSKPSFYRFFLVPGTHVAANPQLDREGHVVPGYEGRPTAPLVGGTQEFAGEHLAMLCGFSPALLAKMLFYLERCDGGVIVGRQEMDVFRYVADSGQTDVPCNLCTFETRHACAHTTLMRLRARHPKFASAARGAIGVFGTMNSAYSDCDVLGNYAAFSALKRADGSENTRTIMQETYRAATERVMAELEALQYVDQAVPTALGRLETIIGTREALHTVVNNIKQLVDREVEQLMRNLIEGRNFKFRDGLAEANHAMSLSLDPYTCGPCPLLQLLARRSNLAVYQDLALSQCHGVFAGQSVEGRNFRNQFQPVLRRRVMDLFNNGFLSAKTLTVALSEGAAICAPSLTAGQTAPAESSFEGDVARVTLGFPKELRVKSRVLFAGASANASEAAKARVASLQSAYQKPDKRVDILLGPLGFLLKQFHAVIFPNGKPPGSNQPNPQWFWTALQRNQLPARLLSREDIETIAFIKRFSLDYGAINFINLAPNNVSELAMYYMANQILRYCDHSTYFINTLTAVIAGSRRPPGVQAAAAWAPQGGAGLEAGARALMDSLDAHPGAWTSMFASCNLLRPVMAARPMVVLGLSISKYYGMAGNDRVFQAGNWASLLGGKNACPLLIFDRTRKFVLACPRAGFVCAASSLGGGAHEHSLCEQLRGIIAEGGAAVASSVFVATVKSLGPRTQQLQIEDWLALLEDEYLSEEMMEFTTRALERGHGEWSTDAALEVAHEAEALVSQLGAAGEVFNFGDFGDEDDHAASFGGLAAAAGAAGVARKRAFHGDDPFGEGPPEKKDLTLDML

SEQ ID NO: 12 - protein, 1197 aa, Human alphaherpesvirus 2
  source 1..1197: note = HSV-2 strain MS
MDTKPKTTTTVKVPPGPMGYVYGRACPAEGLELLSLLSARSGDADVAVAPLIVGLTVESGFEANVAAVVGSRTTGLGGTAVSLKLMPSHYSPSVYVFHGGRHLAPSTQAPNLTRLCERARRHFGFSDYAPRPCDLKHETTGDALCERLGLDPDRALLYLVITEGFREAVCISNTFLHLGGMDKVTIGDAEVHRIPVYPLQMFMPDFSRVIADPFNCNHRSIGENFNYPLPFFNRPLARLLFEAVVGPAAVALRARNVDAVARAAAHLAFDENHEGAALPADITFTAFEASQGKPQRGARDAGNKGPAGGFEQRLASVMAGDAALALESIVSMAVFDEPPPDITTWPLLEGQETPAARAGAVGAYLARAAGLVGAMVFSTNSALHLTEVDDAGPADPKDHSKPSFYRFFLVPGTHVAANPQLDREGHVVPGYEGRPTAPLVGGTQEFAGEHLAMLCGFSPALLAKMLFYLERCDGGVIVGRQEMDVFRYVADSGQTDVPCSLCTFETRHACAHTTLMRLRARHPKFASAARGAIGVFGTMNSAYSDCDVLGNYAAFSALKRADGSENTRTIMQETYRAATERVMAELEALQYVDQAVPTALGRLETIIGNREALHTVVNNIKQLVDREVEQLMRNLIEGRNFKFRDGLAEANHAMSLSLDPYTCGPCPLLQLLARRSNLAVYQDLALSQCHGVFAGQSVEGRNFRNQFQPVLRRRVMDLFNNGFLSAKTLTVALSEGAAICAPSLTAGQTAPAESSFEGDVARVTLGFPKELRVKSRVLFAGASANASEAAKARVASLQSAYQKPDKRVDILLGPLGFLLKQFHAVIFPNGKPPGSNQPNPQWFWTALQRNQLPARLLSREDIETIAFIKRFSLDYGAINFINLAPNNVSELAMYYMANQILRYCDHSTYFINTLTAVIAGSRRPPSVQAAAAWAPQGGAGLEAGARALMDSLDAHPGAWTSMFASCNLLRPVMAARPMVVLGLSISKYYGMAGNDRVFQAGNWASLLGGKNACPLLIFDRTRKFVLACPRAGFVCAASSLGGGAHEHSLCEQLRGIIAEGGAAVASSVFVATVKSLGPRTQQLQIEDWLALLEDEYLSEEMMEFTTRALERGHGEWSTDAALEVAHEAEALVSQLGAAGEVFNFGDFGDEDDHAASFGGLAAAGAGAAGVARKRAFHGDDPFGEGPPEKKDLTLDML

SEQ ID NO: 13 - protein, 1142 aa, Human alphaherpesvirus 2
  source 1..1142: note = HSV-2 strain HG52
MANRPAASALAGARSPSERQEPREPEVAPPGGDHVFCRKVSGVMVLSSDPPGPAAYRISDSSFVQCGSNCSMIIDGDVARGHLRDLEGATSTGAFVAISNVAAGGDGRTAVVALGGTSGPSATTSVGTQTSGEFLHGNPRTPEPQGPQAVPPPPPPPFPWGHECCARRDARGGAEKDVGAAESWSDGPSSDSETEDSDSSDEDTGSETLSRSSSIWAAGATDDDDSDSDSRSDDSVQPDVVVRRRWSDGPAPVAFPKPRRPGDSPGNPGLGAGTGPGSATDPRASADSDSAAHAAAPQADVAPVLDSQPTVGTDPGYPVPLELTPENAEAVARFLGDAVDREPALMLEYFCRCAREESKRVPPRTFGSAPRLTEDDFGLLNYALAEMRRLCLDLPPVPPNAYTPYHLREYATRLVNGFKPLVRRSARLYRILGVLVHLRIRTREASFEEWMRSKEVDLDFGLTERLREHEAQLMILAQALNPYDCLIHSTPNTLVERGLQSALKYEEFYLKRFGGHYMESVFQMYTRIAGFLACRATRGMRHIALGRQGSWWEMFKFFFHRLYDHQIVPSTPAMLNLGTRNYYTSSCYLVNPQATTNQATLRAITGNVSAILARNGGIGLCMQAFNDASPGTASIMPALKVLDSLVAAHNKQSTRPTGACVYLEPWHSDVRAVLRMKGVLAGEEAQRCDNIFSALWMPDLFFKRLIRHLDGEKNVTWSLFDRDTSMSLADFHGEEFEKLYEHLEAMGFGETIPIQDLAYAIVRSAATTGSPFIMFKDAVNRHYIYDTQGAAIAGSNLCTEIVHPASKRSSGVCNLGSVNLARCVSRQTFDFGRLRDAVQACVLMVNIMIDSTLQPTPQCTRGNDNLRSMGIGMQGLHTACLKMGLDLESAEFRDLNTHIAEVMLLAAMKTSNALCVRGARPFSHFKRSMYRAGRFHWERFSNASPRYEGEWEMLRQSMMKHGLRNSQFIALMPTAASAQISDVSEGFAPLFTNLFSKVTRDGETLRPNTLLLKELERTFGGKRLLDAMDGLEAKQWSVAQALPCLDPAHPLRRFKTAFDYDQELLIDLCADRAPYVDHSQSMTLYVTEKADGTLPASTLVRLLVHAYKRGLKTGMYYCKVRKATNSGVFAGDDNIVCTSCAL

SEQ ID NO: 14 - protein, 1144 aa, Human alphaherpesvirus 2
  source 1..1144: note = HSV-2 strain 333
MANRPAASALAGARSPSERQEPREPEVAPPGGDHVFCRKVSGVMVLSSDPPGPAAYRISDSSFVQCGSNCSMIIDGDVARGHLRDLEGATSTGAFVAISNVAAGGDGRTAVVALGGTSGPSATTSVGTQTSGEFLHGNPRTPEPQGPQAVPPPPPPPFPWGHECCARRDARGGAEKDVGAAESWSDGPSSDSETEDSDSSDEDTGSGSETLSRSSSIWAAGATDDDDSDSDSRSDDSVQPDVVVRRRWSDGPAPVAFPKPRRPGDSPGNPGLGAGTGPGSATDPRASADSDSAAHAAAPQADVAPVLDSQPTVGTDPGYPVPLELTPENAEAVARFLGDAVDREPALMLEYFCRCAREESKRVPPRTFGSAPRLTEDDFGLLNYALAEMRRLCLDLPPVPPNAYTPYHLREYATRLVNGFKPLVRRSARLYRILGILVHLRIRTREASFEEWMRSKEVDLDFGLTERLREHEAQLMILAQALNPYDCLIHSTPNTLVERGLQSALKYEEFYLKRFGGHYMESVFQMYTRIAGFLACRATRGMRHIALGRQGSWWEMFKFFFHRLYDHQIVPSTPAMLNLGTRNYYTSSCYLVNPQATTNQATLRAITGNVSAILARNGGIGLCMQAFNDASPGTASIMPALKVLDSLVAAHNKQSTRPTGACVYLEPWHSDVRAVLRMKGVLAGEEAQRCDNIFSALWMPDLFFKRLIRHLDGEKNVTWSLFDRDTSMSLADFHGEEFEKLYEHLEAMGFGETIPIQDLAYAIVRSAATTGSPFIMFKDAVNRHYIYDTQGAAIAGSNLCTEIVHPSSKRSSGVCNLGSVNLARCVSRRTFDFGMLRDAVQACVLMVNIMIDSTLQPTPQCARGHDNLRSMGIGMQGLHTACLKMGLDLESAEFRDLNTHIAEVMLLAAMKTSNALCVRGARPFSHFKRSMYRAGRFHWERFSNASPRYEGEWEMLRQSMMKHGLRNSQFIALMPTAASAQISDVSEGFAPLFTNLFSKVTRDGETLRPNTLLLKELERTFGGKRLLDAMDGLEAKQWSVAQALPCLDPAHPLRRFKTAFDYDQELLIDLCADRAPYVDHSQSMTLYVTEKADGTLPASTLVRLLVHAYKRGLKTGMYYCKVRKATNSGVFAGDDNIVCTSCAL

SEQ ID NO: 15 - protein, 1144 aa, Human alphaherpesvirus 2
  source 1..1144: note = HSV-2 strain MS
MANRPAASALAGARSPSERQEPREPEVAPPGGDHVFCRKVSGVMVLSSDPPGPAAYRISDSSFVQCGSNCSMIIDGDVARGHLRDLEGATSTGAFVAISNVAAGGDGRTAVVALGGTSGPSATTSVGTQTSGEFLHGNPRTPEPQGPQAVPPPPPPPFPWGHECCARRDARGGAEKDVGAAESWSDGPSSDSETEDSDSSDEDTGSGSETLSRSSSIWAAGATDDDDSDSDSRSDDSVQPDVVVRRRWSDGPAPVAFPKPRRPGDSPGNPGLGAGTGPGSATDPRASADSDSAAHAAAPQADVAPVLDSQPTVGTDPGYPVPLELTPENAEAVARFLGDAVDREPALMLEYFCRCAREESKRVPPRTFGSAPRLTEDDFGLLNYALAEMRRLCLDLPPVPPNAYTPYHLREYATRLVNGFKPLVRRSARLYRILGVLVHLRIRTREASFEEWMRSKEVDLDFGLTERLREHEAQLMILAQALNPYDCLIHSTPNTLVERGLQSALKYEEFYLKRFGGHYMESVFQMYTRIAGFLACRATRGMRHIALGRQGSWWEMFKFFFHRLYDHQIVPSTPAMLNLGTRNYYTSSCYLVNPQATTNQATLRAITGNVSAILARNGGIGLCMQAFNDASPGTASIMPALKVLDSLVAAHNKQSTRPTGACVYLEPWHSDVRAVLRMKGVLAGEEAQRCDNIFSALWMPDLFFKRLIRHLDGEKNVTWSLFDRDTSMSLADFHGEEFEKLYEHLEAMGFGETIPIQDLAYAIVRSAATTGSPFIMFKDAVNRHYIYDTQGAAIAGSNLCTEIVHPSCKRSSGVCNLGSVNLARCVSRRTFDFGMLRDAVQACVLMVNIMIDSTLQPTPQCARGHDNLRSMGIGMQGLHTACLKMGLDLESAEFRDLNTHIAEVMLLAAMKTSNALCVRGARPFSHFKRSMYRAGRFHWERFSNASPRYEGEWEMLRQSMMKHGLRNSQFIALMPTAASAQISDVSEGFAPLFTNLFSKVTRDGETLRPNTLLLKELERTFGGKRLLDAMDGLEAKQWSVAQALPCLDPAHPLRRFKTAFDYDQELLIDLCADRAPYVDHSQSMTLYVTEKADGTLPASTLVRLLVHAYKRGLKTGMYYCKVRKATNSGVFAGDDNIVCTSCAL

SEQ ID NO: 16 - protein, 337 aa, Human alphaherpesvirus 2
  source 1..337: note = HSV-2 strain HG52
MDPAVSPASTDPLDTHASGAGAAPIPVCPTPERYFYTSQCPDINHLRSLSILNRWLETELVFVGDEEDVSKLSEGELGFYRFLFAFLSAADDLVTENLGGLSGLFEQKDILHYYVEQECIEVVHSRVYNIIQLVLFHNNDQARRAYVARTINHPAIRVKVDWLEARVRECDSIPEKFILMILIEGVFFAASFAAIAYLRTNNLLRVTCQSNDLISRDEAVHTTASCYIYNNYLGGHAKPEAARVYRLFREAVDIEIGFIRSQAPTDSSILSPGALAAIENYVRFSADRLLGLIHMQPLYSAPAPDASFPLSLMSTDKHTNFFECRSTSYAGAVVNDL

SEQ ID NO: 17 - protein, 337 aa, Human alphaherpesvirus 2; identical to SEQ ID NO: 16
  source 1..337: note = HSV-2 strain 333

SEQ ID NO: 18 - protein, 337 aa, Human alphaherpesvirus 2
  source 1..337: note = HSV-2 strain MS
MDPAVSPASTDPLDTHASGAGAAPIPVCPTPERYFYTSQCPDINHLRSLSILNRWLETELVFVGDEEDVSKLSEGELGFYRFLFAFLSAADDLVTENLGGLSGLFEQKDILHYYVEQECIEVVHSRVYNIIQLVLFHNNDQARRAYVARTINHPAIRVKVDWLEARVRECASIPEKFILMILIEGVFFAASFAAIAYLRINNLLRVTCQSNDLISRDEAVHTTASCYIYNNYLGGHAKPEAARVYRLFREAVDIEIGFIRSQAPTDSSILSPGALAAIENYVRFSADRLLGLIHMQPLYSAPAPDASFPLSLMSTDKHTNFFECRSTSYAGAVVNDL

SEQ ID NO: 19 - protein, 722 aa, Human alphaherpesvirus 2
  source 1..722: note = HSV-2 strain HG52
MQRRARGASSLRLARCLTPANLIRGANAGVPERRIFAGCLLPTPEGLLSAAVGVLRQRADDLQPAFLTGADRSVRLAARHHNTVPESLIVDGLASDPHYDYIRHYASAAKQALGEVELSGGQLSRAILAQYWKYLQTVVPSGLDIPDDPAGDCDPSLHVLLRPTLLPKLLVRAPFKSGAAAAKYAAAVAGLRDAAHRLQQYMFFMRPADPSRPSTDTALRLSELLAYVSVLYHWASWMLWTADKYVCRRLGPADRRFVALSGSLEAPAETFARHLDRGPSGTTGSMQCMALRAAVSDVLGHLTRLAHLWETGKRSGGTYGIVDAIVSTVEVLSIVHHHAQYIINATLTGYVVWASDSLNNEYLTAAVDSQERFCRTAAPLFPTMTAPSWARMELSIKSWFGAALAPDLLRSGTPSPHYESILRLAASGPPGGRGAVGGSCRDKIQRTRRDNAPPPLPRARPHSTPAAPRRCRRHREDLPEPPHVDAADRGPEPCAGRPATYYTHMAGAPPRLPPRNPAPPEQRPAAAARPLAAQREAAGVYDAVRTWGPDAEAEPDQMENTYLLPDDDAAMPAGVGLGATPAADTTAAAAWPAESHAPRAPSEDADSIYESVGEDGGRVYEEIPWVRVYENICPRRRLAGGAALPGDAPDSPYIEAENPLYDWGGSALFSPRRATRAPDPGLSLSPMPARPRTNALANDGPTNVAALSALLTKLKRGRHQSH

SEQ ID NO: 20 - protein, 721 aa, Human alphaherpesvirus 2
  source 1..721: note = HSV-2 strain 333
MQRRARGASSLRLARCLTPANLIRGANAGVPERRIFAGCLLPTPEGLLSAAVGVLRQRADDLQPAFLTGADRSVRLAARHHNTVPESLIVDGLASDPHYDYIRHYASAAKQALGEVELSGGQLSRAILAQYWKYLQTVVPSGLDIPDDPAGDCDPSLHVLLRPTLLPKLLVRAPFKSGAAAAKYAAAVAGLRDAAHRLQQYMFFMRPADPSRPSTDTALRLSELLAYVSVLYHWASWMLWTADKYVCRRLGPADRRFVALSGSLEAPAETFARHLDRGPSGTTGSMQCMALRAAVSDVLGHLTRLAHLWETGKRSGGTYGIVDAIVSTVEVLSIVHHHAQYIINATLTGYVVWASDSLNNEYLRAAVDSQERFCRTAAPLFPTMTAPSWARMELSIKSWFGAALAPDLLRSGTPSPHYESILRLAASGPPGGRGAVGGSCRDKIQRTRRDNAPPPLPRARPHSTPAAPRRFRRHREDLPEPPHVDAADRGPEPCAGRPATYYTHMAGAPPRLPPRNPAPPEQRPAAAARPLAAQREAAGVYDAVRTWGPDAEAEPDQMENTYLLPDDDAAMPAGVGLGATPAADTTAAAWPAESHAPRAPSEDADSIYESVSEDGGRVYEEIPWVRVYENICLRRQDAGGAAPPGDAPDSPYIEAENPLYDWGGSALFSPPGATRAPDPGLSLSPMPARPRTNALANDGPTNVAALSALLTKLKRGRHQSH

SEQ ID NO: 21 - protein, 721 aa, Human alphaherpesvirus 2
  source 1..721: note = HSV-2 strain MS
MQRRARGASSLRLARCLTPANLIRGANAGVPERRIFAGCLLPTPEGLLSAAVGVLRQRADDLQPAFLTGADRSVRLAARHHNTVPESLIVDGLASDPHYDYIRHYASAAKQALGEVELSGGQLSRAILAQYWKYLQTVVPSGLDIPDDPAGDCDPSLHVLLRPTLLPKLLVRAPFKSGAAAAKYAAAVAGLRDATHRLQQYMFFMRPADPSRPSTDTALRLSELLAYVSVLYHWASWMLWTADKYVCRRLGPADRRFVALSGSLEAPAETFARHLDRGPSGTTGSMQCMALRAAVSDVLGHLTRLAHLWETGKRSGGTYGIVDAIVSTVEVLSIVHHHAQYIINATLTGYVVWASDSLNNEYLRAAVDSQERFCRTAAPLFPTMTAPSWARMELSIKSWFGAALAPDLLRSGTPSPHYESILRLAASGPPGGRGAVGGSCRDKIQRTRRDNAPPPLPRARPHSTPAAPRRFRRHREDLPEPPHVDAADRGPEPCAGRPATYYTHMAGAPPRLPPRNPAPPEQRPAAAARPLAAQREAAGVYDAVRTWGPDAEAEPDQMENTYLLPDDDAAMPAGVGLGATPAADTTAAAWPAKGHAPRAPSEDADSIYESVGEDGGRVYEEIPWVRVYENICLRRQDAGGAAPPGDAPDSPYIEAENPLYDWGGSALFSPPGATRAPDPGLSLSPMPARPRTNALANDGPTNVAALSALLTKLKRGRHQSH

SEQ ID NO: 22 - protein, 696 aa, Human alphaherpesvirus 2
  source 1..696: note = HSV-2 strain HG52
MSVRGHAVRRRRASTRSHAPSAHRADSPVEDEPEGGGVGLMGYLRAVFNVDDDSEVEAAGEMASEEPPPRRRREARGHPGSRRASEARAAAPPRRASFPRPRSVTARSQSVRGRRDSAITRAPRGGYLGPMDPRDVLGRVGGSRVVPSPLFLDELNYEEDDYPAAVAHDDGPGARPSATVEILAGRVSGPELQAAFPLDRLTPRVAAWDESVRSALALGHPAGFYPCPDSAFGLSRVGVMHFASPADPKVFFRQTLQQGEALAWYVTGDAILDLTDRRAKTSPSRAMGFLVDAIVRVAINGWVCGTRLHTEGRGSELDDRAAELRRQFASLTALRPVGAAAVPLLSAGGAAPPHPGPDAAVFRSSLGSLLYWPGVRALLGRDCRVAARYAGRMTYIATGALLARFNPGAVKCVLPREAAFAGRVLDVLAVLAEQTVQWLSVVVGARLHPHSAHPAFADVEQEALFRALPLGSPGVVAAEHEALGDTAARRLLATSGLNAVLGAAVYALHTALATVTLKYALACGDARRRRDDAAAARAVLATGLILQRLLGLADTVVACVALAAFDGGSTAPEVGTYTPLRYACVLRATQPLYARTTPAKFWADVRAAAEHVDLRPASSAPRAPVSGTADPAFLLEDLAAFPPAPLNSESVLGPRVRVVDIMAQFRKLLMGDEETAALRAHVSGRRATGLGGPPRP

SEQ ID NO: 23 - protein, 696 aa, Human alphaherpesvirus 2
  source 1..696: note = HSV-2 strain 333
MSVRGHAVRRRRASTRSHAPSAHRADSPVEDEPEGGGVGLMGYLRAVFNVDDDSEVEAAGEMASEEPPPRRRREARGHPGSRRASEARAAAPPRRASFPRPRSVTARSQSVRGRRDSAITRAPRGGYLGPMDPRDVLGRVGGSRVVPSPLFLDELSYEEDDYPAAVAHDDGAGARPPATVEILAGRVSGPELQAAFPLDRLTPRVAAWDESVRSALALGHPAGFYPCPDSAFGLSRVGVMHFASPADPKVFFRQTLQQGEALAWYVTGDAILDLTDRRAKTSPSRAMGFLVDAIVRVAINGWVCGTRLHTEGRGSELDDRAAELRRQFASLTALRPVGAAAVPLLSAGGAAPPHPGPDAAVFRSSLGSLLYWPGVRALLGRDCRVAARYAGRMTYIATGALLARFNPGAVKCVLPREAAFAGRVLDVLAVLAEQTVQWLSVVVGARLHPHSAHPAFADVEQEALFRALPLGSPGVVAAEHEALGDTAARRLLATSGLNAVLGAAVYALHTALATVTLKYALACGDARRRRDDAAAARAVLATGLILQRLLGLADTVVACVALAAFDGGSTAPEVGTYTPLRYACVLRATQPLYARTTPAKFWADVRAAAEHVDLRPASSAPRAPVSGTADPAFLLEDLAAFPPAPLNSESVLGPRVRVVDIMAQFRKLLMGDEETAALRAHVSGRRATGLGGPPRP

SEQ ID NO: 24 - protein, 696 aa, Human alphaherpesvirus 2; identical to SEQ ID NO: 23
  source 1..696: note = HSV-2 strain MS

SEQ ID NO: 25 - protein, 490 aa, Human alphaherpesvirus 2
  source 1..490: note = HSV-2 strain HG52
MDLLVDDLFADADGVSPPPPRPAGGPKNTPAAPPLYATGRLSQAQLMPSPPMPVPPAALFNRLLDDLGFSAGPALCTMLDTWNEDLFSGFPTNADMYRECKFLSTLPSDVIDWGDAHVPERSPIDIRAHGDVAFPTLPATRDELPSYYEAMAQFFRGELRAREESYRTVLANFCSALYRYLRASVRQLHRQAHMRGRNRDLREMLRTTIADRYYRETARLARVLFLHLYLFLSREILWAAYAEQMMRPDLFDGLCCDLESWRQLACLFQPLMFINGSLTVRGVPVEARRLRELNHIREHLNLPLVRSAAAEEPGAPLTTPPVLQGNQARSSGYFMLLIRAKLDSYSSVATSEGESVMREHAYSRGRTRNNYGSTIEGLLDLPDDDDAPAEAGLVAPRMSFLSAGQRPRRLSTTAPITDVSLGDELRLDGEEVDMTPADALDDFDLEMLGDVESPSPGMTHDPVSYGALDVDDFEFEQMFTDAMGIDDFGG

SEQ ID NO: 26 - protein, 490 aa, Human alphaherpesvirus 2; identical to SEQ ID NO: 25
  source 1..490: note = HSV-2 strain 333

SEQ ID NO: 27 - protein, 490 aa, Human alphaherpesvirus 2; identical to SEQ ID NO: 25
  source 1..490: note = HSV-2 strain MS

SEQ ID NO: 28 - protein, 300 aa, Human alphaherpesvirus 2
  source 1..300: note = HSV-2 strain HG52
MTSRRSVKSCPREAPRGTHEELYYGPVSPADPESPRDDFRRGAGPMRARPRGEVRFLHYDEAGYALYRDSSSDDDESRDTARPRRSASVAGSHGPGPARAPPPPGGPVGAGGRSHAPPARTPKMTRGAPKASATPATDPARGRRPAQADSAVLLDAPAPTASGRTKTPAQGLAKKLHFSTAPPSPTAPWTPRVAGFNKRVFCAAVGRLAATHARLAAVQLWDMSRPHTDEDLNELLDLTTIRVTVCEGKNLLQRANELVNPDAAQDVDATAAARGRPAGRAAATARAPARSASRPRRPLE

SEQ ID NO: 29 - protein, 302 aa, Human alphaherpesvirus 2
  source 1..302: note = HSV-2 strain 333
MTSRRSVKSCPREAPRGTHEELYYGPVSPADPESPRDDFRRGAGPMRARPRGEVRFLHYDEAGYALYRDSSSSEDNDESRDTARPRRSASVAGSHGPGPARAPPPPGGPVGAGGRSHAPPARTPKMTRGAPKAPATPATDPARGRRPAQADSAVLLDAPAPTASGRTKTPAQGLAKKLHFSTAPPSPTAPWTPRVAGFNKRVFCAAVGRLAATHARLAAVQLWDMSRPHTDEDLNELLDLTTIRVTVCEGKNLLQRANELVNPDAAQDVDATAAARGRPAGRAAATARAPARSASRPRRPLE

SEQ ID NO: 30 - protein, 302 aa, Human alphaherpesvirus 2
  source 1..302: note = HSV-2 strain MS
MTSRRSVKSCPREAPRGTHEELYYGPVSPADPESPRDDFRRGAGPMRARPRGEVRFLHYDEAGYALYRDSSSSEDDDESRDTARPRRSASVAGSHGPGPARAPPPPGGPVGAGGRSHAPPARTPKMTRGAPKAPATPATDPARGRRPAQADSAVLLDAPAPTASGRTKTPAQGLAKKLHFSTAPPSPTAPWTPRVAGFNKRVFCAAVGRLAATHARLAAVQLWDMSRPHTDEDLNELLDLTTIRVTVCEGKNLLQRANELVNPDAAQDVDATAAARGRPAGRAAATARAPARSASRPRRPLE

SEQ ID NO: 31 - protein, 1318 aa, Human alphaherpesvirus 2
  source 1..1318: note = HSV-2 strain HG52
MSAEQRKKKKTTTTTQGRGAEVAMADEDGGRLRAAAETTGGPGSPDPADGPPPTPNPDRRPAARPGFGWHGGPEENEDEADDAAADADADEAAPASGEAVDEPAADGVVSPRQLALLASMVDEAVRTIPSPPPERDGAQEEAARSPSPPRTPSMRADYGEENDDDDDDDDDDDRDAGRWVRGPETTSAVRGAYPDPMASLSPRPPAPRRHHHHHHHRRRRAPRRRSAASDSSKSGSSSSASSASSSASSSSSASASSSDDDDDDDAARAPASAADHAAGGTLGADDEEAGVPARAPGAAPRPSPPRAEPAPARTPAATAGRLERRRARAAVAGRDATGRFTAGRPRRVELDADAASGAFYARYRDGYVSGEPWPGAGPPPPGRVLYGGLGDSRPGLWGAPEAEEARARFEASGAPAPVWAPELGDAAQQYALITRLLYTPDAEAMGWLQNPRVAPGDVALDQACFRISGAARNSSSFISGSVARAVPHLGYAMAAGRFGWGLAHVAAAVAMSRRYDRAQKGFLLTSLRRAYAPLLARENAALTGARTPDDGGDANRHDGDDARGKPAAAAAPLPSAAASPADERAVPAGYGAAGVLAALGRLSAAPASAPAGADDDDDDDGAGGGGGGRRAEAGRVAVECLAACRGILEALAEGFDGDLAAVPGLAGARPAAPPRPGPAGAAAPPHADAPRLRAWLRELRFVRDALVLMRLRGDLRVAGGSEAAVAAVRAVSLVAGALGPALPRSPRLLSSAAAAAADLLFQNQSLRPLLADTVAAADSLAAPASAPREARKRKSPAPARAPPGGAPRPPKKSRADAPRPAAAPPAGAAPPAPPTPPPRPPRPAALTRRPAEGPDPQGGWRRQPPGPSHTPAPSAAALEAYCAPRAVAELTDHPLFPAPWRPALMFDPRALASLAARCAAPPPGGAPAAFGPLRASGPLRRAAAWMRQVPDPEDVRVVILYSPLPGEDLAAGRAGGGPPPEWSAERGGLSCLLAALGNRLCGPATAAWAGNWTGAPDVSALGAQGVLLLSTRDLAFAGAVEFLGLLAGACDRRLIVVNAVRAADWPADGPVVSRQHAYLACEVLPAVQCAVRWPAARDLRRTVLASGRVFGPGVFARVEAAHARLYPDAPPLRLCRGANVRYRVRTRFGPDTLVPMSPREYRRAVLPALDGRAAASGAGDAMAPGAPDFCEDEAHSHRACARWGLGAPLRPVYVALGRDAVRGGPAELRGPRREFCARALLEPDGDAPPLVLRDDADAGPPPQIRWASAAGRAGTVLAAAGGGVEVVGTAAGLATPPRREPVDMDAELEDDDDGLFGE

SEQ ID NO: 32 - protein, 1322 aa, Human alphaherpesvirus 2
  source 1..1322: note = HSV-2 strain 333
MSAEQRKKKKTTTTTQGRGAEVAMADEDGGRLRAAAETTGGPGSPDPADGPPPTPNPDRRPAARPGFGWHGGPEENEDEDDDAAADADADEAAPASGEAVDEPAADGVVSPRQLALLASMVDEAVRTIPSPPPERDGAEEEAARSPSPPRTPSMCADYGEENDDDDDDDDRDAGRWVRGPETTSAVRGAYPDPMASLSPRPPAPRRHHHHHHRRRRRRAPHRRSTASDSSKSGSSSSASSASSSASSSSSASASSSDDDDDDAARAPASAADHAAGGTLGADDEEAGVPARAPGAAPRPSPPRAEPAPGAGAGSARTPAATAGRLERRRARAAVAGRDATGRFTAGRPRRVELDADAASGAFYARYRDGYVSGEPWPGAGPPPPGRVLYGGLGDSRPGLWGAPEAEEARARFEASGAPAPVWAPELGDAAQQYALITRLLYTPDAEAMGWLQNPRVAPGDVALDQACFRISGAARNSSSFISGSVARAVPHLGYAMAAGRFGWGLAHVAAAVAMSRRYDRAQKGFLLTSLRRAYAPLLARENAALTGARTPDDGGDANRRDGDDARGKPAAAAAPLPSAAASPADERAVPAGYGAAGVLAALGRLSAAPASAPAGADDDDDDDDGAGGGGGGRRAEAGRVAVECLAACRGILEALAEGFDGDLAAVPGLAGARPAAPPRPGPAGAAAPPHADAPRLRAWLRELRFVRDALVLMRLRGDLRVAGGSEAAVAAVRAVSLVAGALGPALPRSPRLLSSAAAAAADLLFQNQSLRPLLADTVAAADSLAAPASAPREARKRKSPAPARAPPGGAPRPPKKSRADAPRPAAAPPAGAAPPAPPTPPPRPPRPAALTRRPAEGPDPQGGWRRQPPGPSHTPAPSAAALEAYCAPRAVAELTDHPLFPAPWRPALMFDPRALASLAARCAAPPPGGAPAAFGPLRASGPLRRAAAWMRQVPDPEDVRVVILYSPLPGEDLAAGRAGGGPPPEWSAERGGLSCLLAALGNRLCGPATAAWAGNWTGAPDVSALGAQGVLLLSTRDLAFAGAVEFLGLLAGACDRRLIVVNAVRAADWPADGPVVSRQHAYLACEVLPAVQCAVRWPAARDLRRTVLASGRVFGPGVFARVEAAHARLYPDAPPLRLCRGANVRYRVRTRFGPDTLVPMSPREYRRAVLPALDGRAAASGAGDAMAPGAPDFCEDEAHSHRACARWGLGAPLRPVYVALGRDAVRGGPAELRGPRREFCARALLEPDGDAPPLVLRDDADAGPPPQIRWASATGRAGTVLAAAGGGVEVVGTAAGLATPPRREPVDMDAELEDDDDGLFGE

SEQ ID NO: 33 - protein, 1331 aa, Human alphaherpesvirus 2
  source 1..1331: note = HSV-2 strain MS
MSAEQRKKKKTTTTQGRGAEVAMADEDGGRLRAAAETTGGPGSPDPADGPPPTPNPDRRPAARPGFGWHGGPEENEDEADGAAADADADEAAPASGEAVDEPAADGVVSPRQLALLASMVDEAVRTIPSPPPERDGAEEEAARSPSPPRTPSMRADYGEENDDDDDDDDDDDRDAGRWVRGPETTSAVRGAYPDPMASLSPRPPAPRRHHHHHHHRRRRAPRRRSAASDSSKSGSSSSASSASSSASSSSSASASSSDDDDDDAARAPASAADHAADHAADHAADHAADHAADHAAGGTLGADDEEAGVPARAPGAAPRPSPPRAEPAPGAGAGSARTPAATAGRLERRRARAAVAGRDATGRFTAGRPRRVELDADAASGAFYARYRDGYVSGEPWPGAGPPPPGRVLYGGLGDSRPGLWGAPEAEEARARFEASGAPAPVWAPELGDAAQQYALITRLLYTPDAEAMGWLQNPRVAPGDVALDQACFRISGAARNSSSFISGSVARAVPHLGYAMAAGRFGWGLAHVAAAVAMSRRYDRAQKGFLLTSLRRAYAPLLARENAALTGARTPDDGDDARGKPAAAAPLPSAAASPADERAVPAGYGAAGVLAALGRLSAAPASAPAGADDDDDDDGAGGGGGGGRRAEAGRVAVECLAACRGILEALAEGFDGDLAAVPGLAGARPAAPPRPGPAGAAAPPHADAPRLRAWLRELRFVRDALVLMRLRGDLRVAGGSEAAVAAVRAVSLVAGALGPALPRSPRLLSSAAAAAADLLFQNQSLRPLLADTVAAADSLAAPASAPREARKRKSPAPARAPPGGAPRPPKKSRADAPRPAAAPPAGAAPPAPPTPPPRPAALTRRPAEGPDPQGGWRRQPPGPSHTPAPSAAALEAYCAPRAVAELTDHPLFPAPWRPALMFDPRALASLAARCAAPPPGGAPAAFGPLRASGPLRRAAAWMRQVPDPEDVRVVILYSPLPGEDLAAGRAGGGPPPEWSAERGGLSCLLAALGNRLCGPATAAWAGNWTGAPDVSALGAQGVLLLSTRDLAFAGAVEFLGLLAGACDRRLIVVNAVRAADWPADGPVVSRQHAYLACEVLPAVQCAVRWPAARDLRRTVLASGRVFGPGVFARVEAAHARLYPDAPPLRLCRGANVRYRVRTRFGPDTLVPMSPREYRRAVLPALDGRAAASGAGDAMAPGAPDFCEDEAHSHRACARWGLGAPLRPVYVALGRDAVRGGPAELRGPRREFCARALLEPDGDAPPLVLRDDADAGPPPQIRWASAAGRAGTVLAAAGGGVEVVGAAAGLATPPRREPVDMDAELEDDDDGLFGE

SEQ ID NO: 34 - protein, 825 aa, Human alphaherpesvirus 2
  source 1..825: note = HSV-2 strain HG52
MEPRPGTSSRADPGPERPPRQTPGTQPAAPHAWGMLNDMQWLASSDSEEETEVGISDDDLHRDSTSEAGSTDTEMFEAGLMDAATPPARPPAERQGSPTPADAQGSCGGGPVGEEEAEAGGGGDVCAVCTDEIAPPLRCQSFPCLHPFCIPCMKTWIPLRNTCPLCNTPVAYLIVGVTASGSFSTIPIVNDPRTRVEAEAAVRAGTAVDFIWTGNPRTAPRSLSLGGHTVRALSPTPPWPGTDDEDDDLADVDYVPPAPRRAPRRGGGGAGATRGTSQPAATRPAPPGAPRSSSSGGAPLRAGVGSGSGGGPAVAAVVPRVASLPPAAGGGRAQARRVGEDAAAAEGRTPPARQPRAAQEPPIVISDSPPPSPRRPAGPGPLSFVSSSSAQVSSGPGGGGLPQSSGRAARPRAAVAPRVRSPPRAAAAPVVSASADAAGPAPPAVPVDAHRAPRSRMTQAQTDTQAQSLGRAGATDARGSGGPGAEGGPGVPRGTNTPGAAPHAAEGAAARPRKRRGSDSGPAASSSASSSAAPRSPLAPQGVGAKRAAPRRAPDSDSGDRGHGPLAPASAGAAPPSASPSSQAAVAAASSSSASSSSASSSSASSSSASSSSASSSSASSSSASSSAGGAGGSVASASGAGERRETSLGPRAAAPRGPRKCARKTRHAEGGPEPGARDPAPGLTRYLPIAGVSSVVALAPYVNKTVTGDCLPVLDMETGHIGAYVVLVDQTGNVADLLRAAAPAWSRRTLLPEHARNCVRPPDYPTPPASEWNSLWMTPVGNMLFDQGTLVGALDFHGLRSRHPWSREQGAPAPAGDAPAGHGE

SEQ ID NO: 35 - protein, 814 aa, Human alphaherpesvirus 2
  source 1..814: note = HSV-2 strain 333
MEPRPGTSSRADPGPERPPRQTPGTQPAAPHAWGMLNDMQWLASSDSEEETEVGISDDDLHRDSTSEAGSTDTEMFEAGLMDAATPPARPPAERQGSPTPADAQGSCGGGPVGEEEAEAGGGGDVCAVCTDEIAPPLRCQSFPCLHPFCIPCMKTWIPLRNTCPLCNTPVAYLIVGVTASGSFSTIPIVNDPRTRVEAEAAVRSGTAVDFIWTGNPRTAPRSLSLGGHTVRALSPTPPWPGTDDEDDDLADADYVPPAPRRAPRRGGGGAGATRGTSQPAATRPAPPGAPRSSSSGGAPLRAGVGSGSGGGPAVAAVVPRVASLPPAAGGGRAQARRVGEDAAAAEGRTPPARQPRAAQEPPIVISDSPPPSPRRPAGPGPLSFVSSSSAQVSSGPGGGGLPQSSGRAARPRAAVAPRVRSPPRAAAAPVVSASADAAGPAPPAVPVDAHRAPRSRMTQAQTDTQAQSLGRAGATDARGSGGPGAEGGPGVPRGTNTPGAAPHAAEGAAARPRKRRGSDSGPAASSSASSSAAPRSPLAPQGVGAKRAAPRRAPDSDSGGRGHGPLAPASAGAAPPSASPSSQAAVAAAAASSSSSSSASSSSSSASSSSSSASSSAGGAGGSVASASGAGERRETSLGPRAAAPRGPRKCARKTRHAEGGPEPGARDPAPGLTRYLPIAGVSSVVALAPYVNKTVTGDCLPVLDMETGHIGAYVVLVDQTGNVADLLRAAAPAWSRRTLLPEHARNCVRPPDYPTPPASEWNSLWMTPVGNMLFDQGTLVGALDFHGLRSRHPWSREQGAPAPAGDAPAGHGE

SEQ ID NO: 36 - protein, 801 aa, Human alphaherpesvirus 2
  source 1..801: note = HSV-2 strain MS
MEPRPGTSSRADPGPERPPRQTPGTQPAAPHAWGMLNDMQWLASSDSEEETEVGISDDDLHRDSTSEAGSTDTEMFEAGLMDAATPPARPPAERQGSPTPADAQGSCGGGPVGEEEAEAGGGGDVCAVCTDEIAPPLRCQSFPCLHPFCIPCMKTWIPLRNTCPLCNTPVAYLIVGVTASGSFSTIPIVNDPRTRVEAEAAVRSGTAVDFIWTGNPRTAPRSLSLGGHTVRALSPTPPWPGTDDEDDDLADADYVPPAPRRAPRRGGGGAGATRGTSQPAATRPAPPGAPRSSSSGGAPLRAGVGSGSGGGPAVAAVVPRVASLPPAAGGGRAQARRVGEDAAAAEGRTPPARQPRAAQEPPIVISDSPPPSPRRPAGPGPLSFFSSSSAQVSSGPGGGGLPQSSGRAARPRAAVAPRVRSPPRAAAAPVVSASADAAGPAPPAVPVDAHRAPRSRMTQAQTDTQAQSLGRAGATDARGSGGPGAEGGPGVPRGTNTPGAAPHAAEGAAARPRKRRGSDSGPAASSSASSSAAPRSPLAPQGVGAKRAAPRRAPDSDSGDRGHGPLAPASAGAAPPSASPSSQAAVAAAASSSSSSSSSASSSAGGAGGSVASASGAGERRETSLGPRAAAPRGPRKCARKTRHAEGGPEPGARDPAPGLTRYLPIAGASSVVALAPYVNKTVTGDCLPILDMETGHIGAYVVLVDQTGNVADLLRAAAPAWSRRTLLPEHARNCVRPPDYPTPPASEWNSLWMTPVGNMLFDQGTLVGALDFHGLRSRHPWSREQGAPAPAGDAPAGHGE

SEQ ID NO: 37 - protein, 881 aa, Human alphaherpesvirus 2
  source 1..881: note = HSV-2 strain HG52
MAASGGEGSRDVRAPGPPPQQPGARPAVRFRDEAFLNFTSMHGVQPIIARIRELSQQQLDVTQVPRLQWFRDVAALEVPTGLPLREFPFAAYLITGNAGSGKSTCVQTLNEVLDCVVTGATRIAAQNMYVKLSGAFLSRPINTIFHEFGFRGNHVQAQLGQHPYTLASSPASLEDLQRRDLTYYWEVILDITKRALAAHGGEDARNEFHALTALEQTLGLGQGALTRLASVTHGALPAFTRSNIIVIDEAGLLGRHLLTTVVYCWWMINALYHTPQYAGRLRPVLVCVGSPTQTASLESTFEHQKLRCSVRQSENVLTYLICNRTLREYTRLSHSWAIFINNKRCVEHEFGNLMKVLEYGLPITEEHMQFVDRFVVPESYITNPANLPGWTRLFSSHKEVSAYMAKLHAYLKVTREGEFVVFTLPVLTFVSVKEFDEYRRLTQQPTLTMEKWITANASRITNYSQSQDQDAGHVRCEVHSKQQLVVARNDITYVLNSQVAVTARLRKMVFGFDGTFRTFEAVLRDDSFVKTQGETSVEFAYRFLSRLMFGGLIHFYNFLQRPGLDATQRTLAYGRLGELTAELLSLRRDAAGASATRAADTSDRSPGERAFNFKHLGPRDGGPDDFPDDDLDVIFAGLDEQQLDVFYCHYALEEPETTAAVHAQFGLLKRAFLGRYLILRELFGEVFESAPFSTYVDNVIFRGCELLTGSPRGGLMSVALQTDNYTLMGYTYTRVFAFAEELRRRHATAGVAEFLEESPLPYIVLRDQHGFMSVVNTNISEFVESIDSTELAMAINADYGISSKLAMTITRSQGLSLDKVAICFTPGNLRLNSAYVAMSRTTSSEFLHMNLNPLRERHERDDVISEHILSALRDPNVVIVY

SEQ ID NO: 38 - protein, 881 aa, Human alphaherpesvirus 2; identical to SEQ ID NO: 37
  source 1..881: note = HSV-2 strain 333

SEQ ID NO: 39 - protein, 881 aa, Human alphaherpesvirus 2; identical to SEQ ID NO: 37
  source 1..881: note = HSV-2 strain MS

SEQ ID NO: 40 - protein, 867 aa, Human alphaherpesvirus 2
  source 1..867: note = HSV-2 strain HG52
MNVATCTHQTHHAARAPGATSAPGAASGDPLGARRPIGDDECEQYTSSVSLARMLYGGDLAEWVPRVHPKTTIERQQHGPVTFPDASAPTARCVTVVRAPMGSGKTTALIRWLGEAIHSPDTSVLVVSCRRSFTQTLATRFAESGLPDFVTYFSSTNYIMNDRPFHRLIVQVESLHRVGPNLLNNYDVLVLDEVMSTLGQLYSPTMQQLGRVDALMLRLLRTCPRIIAMDATANAQLVDFLCSLRGEKNVHVVIGEYAMPGFSARRCLFLPRLGPEVLQAALRRRGPAGGAPPPDAPPDATFFGELEARLAGGDNVCIFSSTVSFAEVVARFCRQFTDRVLLLHSLTPPGDVTTWGRYRVVIYTTVVTVGLSFDPPHFDSMFAYVKPMNYGPDMVSVYQSLGRVRTLRKGELLIYMDGSGARSEPVFTPMLLNHVVSASGQWPAQFSQVTNLLCRRFKGRCDASHADAAQARGSRIYSKFRYKHYFERCTLACLADSLNILHMLLTLNCMHVRFWGHDAALTPRNFCLFLRGIHFDALRAQRDLRELRCQDPDTSLSAQAAETEEVGLFVEKYLRPDVAPAEVVALMRGLNSLVGRTRFIYLVLLEACLRVPMAAHSSAIFRRLYDHYATGVIPTINAAGELELVALHPTLNVAPVWELFRLCSTMAACLQWDSMAGGSGRTFSPEDVLELLNPHYDRYMQLVFELGHCNVTDGPLLSEDAVKRVADALSGCPPRGSVSETEHALSLFKIIWGELFGVQLAKSTQTFPGAGRVKNLTKRAIVELLDAHRIDHSACRTHRQLYALLMAHKREFAGARFKLRAPAWGRCLRTHASGAQPNTDIILEAALSELPTEAWPMMQGAVNFSTL

SEQ ID NO: 41 - protein, 871 aa, Human alphaherpesvirus 2
  source 1..871: note = HSV-2 strain 333
MAETMNVATCTHQTHHAARAPGATSAPGAASGDPPGARRPIGDDECEQYTSSVSLARMLYGGDLAEWVPRVHPKTTIERQQHGPVTFPDASAPTARCVTVVRAPMGSGKTTALIRWLGEAIHSPDTSVLVVSCRRSFTQTLATRFAESGLPDFVTYFSSTNYIMNDRPFHRLIVQVESLHRVGPNLLNNYDVLVLDEVMSTLGQLYSPTMQQLGRVDALMLRLLRTCPRIIAMDATANAQLVDFLCSLRGEKNVHVVIGEYAMPGFSARRCLFLPRLGPEVLQAALRPPGPAGGAPPPDAPPDATFFGELEARLAGGDNVCIFSSTVSFAEVVARFCRQFTDRVLLLHSLTPPGDVTTWGRYRVVIYTTVVTVGLSFDPPHFDSMFAYVKPMNYGPDMVSVYQSLGRVRTLRKGELLIYMDGSGARSEPVFTPMLLNHVVSASGQWPAQFSQVTNLLCRRFKGRCDASHADAAQARGSRIYSKFRYKHYFERCTLACLADSLNILHMLLTLNCMHVRFWGHDAALTPRNFCLFLRGIHFDALRAQRDLRELRCQDPDTSLSAQAAETEEVGLFVEKYLRPDVAPAEVVALMRGLNSLVGRTRFIYLVLLEACLRVPMAAHSSAIFRRLYDHYATGVIPTINAAGELELVALHPTLNVAPVWELFRLCSTMAACLQWDSMAGGSGRTFSPEDVLELLNPHYDRYMQLVFELGHCNVTDGPLLSEDAVKRVADALSGCPPRGSVSETEHALSLFKIIWGELFGVQLAKSTQTFPGAGRVKNLTKRAIVELLDAHRIDHSACRTHRQLYALLMAHKREFAGARFKLRAPAWGRCLRTHASGAQPNTDIILEAALSELPTEAWPMMQGAVNFSTL

SEQ ID NO: 42 - protein, 871 aa, Human alphaherpesvirus 2
  source 1..871: note = HSV-2 strain MS
MAETMNVATCTHQTHHAARAPGATSAPGAASGDPLGARRPIGDDECEQYTSSVSLARMLYGGDLAEWVPRVHPKTTIERQQHGPVTFPDASAPTARCVTVVRAPMGSGKTTALIRWLGEAIHSPDTSVLVVSCRRSFTQTLATRFAESGLPDFVTYFSSTNYIMNDRPFHRLIVQVESLHRVGPNLLNNYDVLVLDEVMSTLGQLYSPTMQQLGRVDALMLRLLRTCPRIIAMDATANAQLVDFLCSLRGEKNVHVVIGEYAMPGFSARRCLFLPRLGPEVLQAALRPPGPAGGAPPPDAPPDATFFGELEARLAGGDNVCIFSSTVSFAEVVARFCRQFTDRVLLLHSLTPPGDVTTWGRYRVVIYTTVVTVGLSFDPPHFDSMFAYVKPMNYGPDMVSVYQSLGRVRTLRKGELLIYMDGSGARSEPVFTPMLLNHVVSASGQWPAQFSQVTNLLCRRFKGRCDASRADAAQARGSRIYSKFRYKHYFERCTLACLADSLNILHMLLTLNCMHVRFWGHDAALTPRNFCLFLRGIHFDALRAQRDLRELRCQDPDTSLSAQAAETEEVGLFVEKYLRPDVAPAEVVALMRGLNSLVGRTRFIYLVLLEACLRVPMAAHSSAIFRRLYDHYATGVIPTINAAGELELVALHPTLNVAPVWELFRLCSTMAACLQWDSMAGGSGRTFSPEDVLELLNPHYDRYMQLVFELGHCNVTDGPLLSEDAVKRVADALSGCPPRGSVSETEHALSLFKIIWGELFGVQLAKSTQTFPGAGRVKNLTKRAIVELLDAHRIDHSACRTHRQLYALLMAHKREFAGARFKLRAPAWGRCLRTHASGAQPNTDIILEAALSELPTEAWPMMQGAVNFSTL

SEQ ID NO: 43 - protein, 1374 aa, Human alphaherpesvirus 2
  source 1..1374: note = HSV-2 strain HG52
MAAPARDPPGYRYAAAILPTGSILSTIEVASHRRLFDFFAAVRSDENSLYDVEFDALLGSYCNTLSLVRFLELGLSVACVCTKFPELAYMNEGRVQFEVHQPLIARDGPHPVEQPVHNYMTKVIDRRALNAAFSLATEAIALLTGEALDGTGISLHRQLRAIQQLARNVQAVLGAFERGTADQMLHVLLEKAPPLALLLPMQRYLDNGRLATRVARATLVAELKRSFCDTSFFLGKAGHRREAIEAWLVDLTTATQPSVAVPRLTHADTRGRPVDGVLVTTAAIKQRLLQSFLKVEDTEADVPVTYGEMVLNGANLVTALVMGKAVRSLDDVGRHLLDMQEEQLEANRETLDELESAPQTTRVRADLVAIGDRLVFLEALERRIYAATNVPYPLVGAMDLTFVLPLGLFNPAMERFAAHAGDLVPAPGHPEPRAFPPRQLFFWGKDHQVLRLSMENAVGTVCHPSLMNIDAAVGGVNHDPVEAANPYGAYVAAPAGPGADMQQRFLNAWRQRLAHGRVRWVAECQMTAEQFMQPDNANLALELHPAFDFFAGVADVELPGGEVPPAGPGAIQATWRVVNGNLPLALCPVAFRDARGLELGVGRHAMAPATIAAVRGAFEDRSYPAVFYLLQAAIHGNEHVFCALARLVTQCITSYWNNTRCAAFVNDYSLVSYIVTYLGGDLPEECMAVYRDLVAHVEALAQLVDDFTLPGPELGGQAQAELNHLMRDPALLPPLVWDCDGLMRHAALDRHRDCRIDAGGHEPVYAAACNVATADFNRNDGRLLHNTQARAADAADDRPHRPADWTVHHKIYYYVLVPAFSRGRCCTAGVRFDRVYATLQNMVVPEIAPGEECPSDPVTDPAHPLHPANLVANTVKRMFHNGRVVVDGPAMLTLQVLAHNMAERTTALLCSAAPDAGANTASTANMRIFDGALHAGVLLMAPQHLDHTIQNGEYFYVLPVHALFAGADHVANAPNFPPALRDLARDVPLVPPALGANYFSSIRQPVVQHARESAAGENALTYALMAGYFKMSPVALYHQLKTGLHPGFGFTVVRQDRFVTENVLFSERASEAYFLGQLQVARHETGGGVNFTLTQPRGNVDLGVGYTAVAATGTVRNPVTDMGNLPQNFYLGRGAPPLLDNAAAVYLRNAVVAGNRLGPAQPLPVFGCAQVPRRAGMDHGQDAVCEFIATPVATDINYFRRPCNPRGRAAGGVYAGDKEGDVIALMYDHGQSDPARPFAATANPWASQRFSYGDLLYNGAYHLNGASPVLSPCFKFFTAADITAKHRCLERLIVETGSAVSTATAASDVQFKRPPGCRELVEDPCGLFQEAYPITCASDPALLRSARDGEAHARETHFTQYLIYDASPLKGLSL

SEQ ID NO: 44 - protein, 1374 aa, Human alphaherpesvirus 2
  source 1..1374: note = HSV-2 strain 333
MAAPARDPPGYRYAAAMVPTGSILSTIEVASHRRLFDFFARVRSDENSLYDVEFDALLGSYCNTLSLVRFLELGLSVACVCTKFPELAYMNEGRVQFEVHQPLIARDGPHPVEQPVHNYMTKVIDRRALNAAFSLATEAIALLTGEALDGTGISLHRQLRAIQQLARNVQAVLGAFERGTADQMLHVLLEKAPPLALLLPMQRYLDNGRLATRVARATLVAELKRSFCDTSFFLGKAGHRREAIEAWLVDLTTATQPSVAVPRLTHADTRGRPVDGVLVTTAAIKQRLLQSFLKVEDTEADVPVTYGEMVLNGANLVTALVMGKAVRSLDDVGRHLLEMQEEQLEANRETLDELESAPQTTRVRADLVAIGDRLVFLEALEKRIYAATNVPYPLVGAMDLTFVLPLGLFNPAMERFAAHAGDLVPAPGHPEPRAFPPRQLFFWGKDHQVLRLSMENAVGTVCHPSLMNIDAAVGGVNHDPVEAANPYGAYVAAPAGPGADMQQRFLNAWRQRLAHGRVRWVAECQMTAEQFMQPDNANLALELHPAFDFFAGVADVELPGGEVPPAGPGAIQATWRVVNGNLPLALCPVAFRDARGLELGVGRHAMAPATIAAVRGAFEDRSYPAVFYLLQAAIHGSEHVFCALARLVTQCITSYWNNTRCAAFVNDYSLVSYIVTYLGGDLPEECMAVYRDLVAHVEALAQLVDDFTLPGPELGGQAQAELNHLMRDPALLPPLVWDCDGLMRHAALDRHRDCRIDAGGHEPVYAAACNVATADFNRNDGRLLHNTQARAADAADDRPHRPADWTVHHKIYYYVLVPAFSRGRCCTAGVRFDRVYATLQNMVVPEIAPGEECPSDPVTDPAHPLHPANLVANTVNAMFHNGRVVVDGPAMLTLQVLAHNMAERTTALLCSAAPDAGANTASTANMRIFDGALHAGVLLMAPQHLDHTIQNGEYFYVLPVHALFAGADHVANAPNFPPALRDLARHVPLVPPALGANYFSSIRQPVVQHARESAAGENALTYALMAGYFKMSPVALYHQLKTGLHPGFGFTVVRQDRFVTENVLFSERASEAYFLGQLQVARHETGGGVSFTLTQPRGNVDLGVGYTAVAATATVRNPVTDMGNLPQNFYLGRGAPPLLDNAAAVYLRNAVVAGNRLGPAQPLPVFGCAQVPRRAGMDHGQDAVCEFIATPVATDINYFRRPCNPRGRAAGGVYAGDKEGDVIALMYDHGQSDPARPFAATANPWASQRFSYGDLLYNGAYHLNGASPVLSPCFKFFTAADITAKHRCLERLIVETGSAVSTATAASDVQFKRPPGCRELVEDPCGLFQEAYPITCASDPALLRSARDGEAHARETHFTQYLIYDASPLKGLSL

SEQ ID NO: 45 - protein, 1374 aa, Human alphaherpesvirus 2
  source 1..1374: note = HSV-2 strain MS
MAAPARDPPGYRYAAAMVPTGSILSTIEVASHRRLFDFFARVRSDENSLYDVEFDALLGSYCNTLSLVRFLELGLSVACVCTKFPELAYMNEGRVQFEVHQPLIARDGPHPVEQPVHNYMTKVIDRRALNAAFSLATEAIALLTGEALDGTGISLHRQLRAIQQLARNVQAVLGAFERGTADQMLHVLLEKAPPLALLLPMQRYLDNGRLATRVARATLVAELKRSFCDTSFFLGKAGHRREAIEAWLVDLTTATQPSVAVPRLTHADTRGRPVDGVLVTTAAIKQRLLQSFLKVEDTEADVPVTYGEMVLNGANLVTALVMGKAVRSLDDVGRHLLEMQEEQLEANRETLDELESAPQTTRVRADLVAIGDRLVFLEALEKRIYAATNVPYPLVGAMDLTFVLPLGLFNPAMERFAAHAGDLVPAPGHPEPRAFPPRQLFFWGKDHQVLRLSMENAVGTVCHPSLMNIDAAVGGVNHAPVEAANPYGAYVAAPAGPGADMQQRFLNAWRQRLAHGRVRWVAECQMTAEQFMQPDNANLALELHPAFDFFAGVADVELPGGEVPPAGPGAIQATWRVVNGNLPLALCPVAFRDARGLELGVGRHAMAPATIAAVRGAFEDRSYPAVFYLLQAAIHGSEHVFCALARLVTQCITSYWNNTRCAAFVNDYSLVSYIVTYLGGDLPEECMAVYRDLVAHVEALAQLVDDFTLPGPELGGQAQAELNHLMRDPALLPPLVWDCDGLMRHAALDRHRDCRIDAGGHEPVYAAACNVATADFNRNDGRLLHNTQARAADAADDRPHRPADWTVHHKIYYYVLVPAFSRGRCCTAGVRFDRVYATLQNMVVPEIAPGEECPSDPVTDPAHPLHPANLVANTVNAMFHNGRVVVDGPAMLTLQVLAHNMAERTTALLCSAAPDAGANTASTANMRIFDGALHAGVLLMAPQHLDHTIQNGEYFYVLPVHALFAGADHVANAPNFPPALRDLARHVPLVPPALGANYFSSIRQPVVQHARESAAGENALTYALMAGYFKMSPVALYHQLKTGLHPGFGFTVVRQDRFVTENVLFSERASEAYFLGQLQVARHETGGGVSFTLTQPRGNVDLGVGYTAVAATATVRNPVTDMGNLPQNFYLGRGAPPLLDNAAAVYLRNAVVAGNRLGPAQPLPVFGCAQVPRRAGMDHGQDAVCEFIATPVATDINYFRRPCNPRGRAAGGVYAGDKEGDVIALMYDHGQSDPARPFAATANPWASQRFSYGDLLYNGAYHLNGASPVLSPCFKFFTAADITAKHRCLERLIVETGSAVSTATAASDVQFKRPPGCRELVEDPCGLFQEAYPITCASDPALLRSARDGEAHARETHFTQYLIYDASPLKGLSL

SEQ ID NO: 46 - protein, 585 aa, Human alphaherpesvirus 2
  source 1..585: note = HSV-2 strain HG52
MDPYYPFDALDVWEHRRFIVADSRSFITPEFPRDFWMLPVFNIPRETAAERAAVLQAQRTAAAAALENAALQAAELPVDIERRIRPIEQQVHHIADALEALETAAAAAEEADAARDAEARGEGAADGAAPSPTAGPAAAEMEVQIVRNDPPLRYDTNLPVDLLHMVYAGRGAAGSSGVVFGTWYRTIQERTIADFPLTTRSADFRDGRMSKTFMTALVLSLQSCGRLYVGQRHYSAFECAVLCLYLLYRTTHESSPDRDRAPVAFGDLLARLPRYLARLAAVIGDESGRPQYRYRDDKLPKAQFAAAGGRYEHGALATHVVIATLVRHGVLPAAPGDVPRDTSTRVNPDDVAHRDDVNRAAAAFLARGHNLFLWEDQTLLRATANTITALAVLRRLLANGNVYADRLDNRLQLGMLIPGAVPAEAIARGASGLDSGAIKSGDNNLEALCVNYVLPLYQADPTVELTQLFPGLAALCLDAQAGRPLASTRRVVDMSSGARQAALVRLTALELINRTRTNTTPVGEIINAHDALGIQYEQGPGLLAQQARIGLASNTKRFATFNVGSDYDLLYFLCLGFIPQYLSVA

SEQ ID NO: 47 - protein, 585 aa, Human alphaherpesvirus 2
  source 1..585: note = HSV-2 strain 333
MDPYYPFDALDVWEHRRFIVADSRSFITPEFPRDFWMLPVFNIPRETAAERAAVLQAQRTAAAAALENAALQAAELPVDIERRIRPIEQQVHHIADALEALETAAAAAEEADAARDAEARGEGAADGAAPSPTAGPAAAEMEVQIVRNDPPLRYDTNLPVDLLHMVYAGRGAAGSSGVVFGTWYRTIQERTIADFPLTTRSADFRDGRMSKTFMTALVLSLQSCGRLYVGQRHYSAFECAVLCLYLLYRTTHESSPDRDRAPVAFGDLLARLPRYLARLAAVIGDESGRPQYRYRDDKLPKAQFAAAGGRYEHGALATHVVIATLVRHGVLPAAPGDVPRDTSTRVNPDDVAHRDDVNRAAAAFLARGHNLFLWEDQTLLRATANTITALAVLRRLLANGNVYADRLDNRLQLGMLIPGAVPAEAIARGASGLDSGAIKSGDNNLEALCVNYVLPLYQADPTVELTQLFPGLAALCLDAQAGRPLASTRRVVDMSSGARQAALVRLTALELINRTRTNTTPVGEIINAHDALGIQYEQGLGLLAQQARIGLASNAKRFATFNVGSDYDLLYFLCLGFIPQYLSVA

SEQ ID NO: 48 - protein, 585 aa, Human alphaherpesvirus 2
  source 1..585: note = HSV-2 strain MS
MDPYYPFDALDVWEHRRFIVADSRSFITPEFPRDFWMLPVFNIPRETAAERAAVLQAQRTAAAAALENAALQAAELPVDIERRIRPIEQQVHHIADALEALETAAAAAEEADAARDAEARGEGAADGAAPSPTAGPAAAEMEVQIVRNDPPLRYDTNLPVDLLHMVYAGRGAAGSSGVVFGTWYRTIQERTIADFPLTTRSADFRDGRMSKTFMTALVLSLQSCGRLYVGQRHYSAFECAVLCLYLLYRTTHESSPDRDRAPVAFGDLLARLPCYLARLAAVIGDESGRPQYRYRDDKLPKAQFAAAGGRYEHGALATHVVIATLVRHGVLPAAPGDVPRDTSTRVNPDDVAHRDDVNRAAAAFLARGHNLFLWEDQTLLRATANTITALAVLRRLLANGNVYADRLDNRLQLGMLIPGAVPAEAIARGASGLDSGAIKSGDNNLEALCVNYVLPLYQADPTVELTQLFPGLAALCLDAQAGRPLASTRRVVDMSSGARQAALVRLTALELINRTRTNTTPVGEIINAHDALGIQYEQGLGLLAQQARIGLASNAKRFATFNVGSDYDLLYFLCLGFIPQYLSVA

SEQ ID NO: 49 - protein, 1240 aa, Human alphaherpesvirus 2
  source 1..1240: note = HSV-2 strain HG52
MFCAAGGPASPGGKPAARAASGFFAPHNPRGATQTAPPPCRRQNFYNPHLAQTGTQPKALGPAQRHTYYSECDEFRFIAPRSLDEDAPAEQRTGVHDGRLRRAPKVYCGGDERDVLRVGPEGFWPRRLRLWGGADHAPEGFDPTVTVFHVYDILEHVEHAYSMRAAQLHERFMDAITPAGTVITLLGLTPEGHRVAVHVYGTRQYFYMNKAEVDRHLQCRAPRDLCERLAAALRESPGASFRGISADHFEAEVVERADVYYYETRPTLYYRVFVRSGRALAYLCDNFCPAIRKYEGGVDATTRFILDNPGFVTFGWYRLKPGRGNAPAQPRPPTAFGTSSDVEFNCTADNLAVEGAMCDLPAYKLMCFDIECKAGGEDELAFPVAERPEDLVIQISCLLYDLSTTALEHILLFSLGSCDLPESHLSDLASRGLPAPVVLEFDSEFEMLLAFMTFVKQYGPEFVTGYNIINFDWPFVLTKLTEIYKVPLDGYGRMNGRGVFRVWDIGQSHFQKRSKIKVNGMVNIDMYGIITDKVKLSSYKLNAVAEAVLKDKKKDLSYRDIPAYYASGPAQRGVIGEYCVQDSLLVGQLFFKFLPHLELSAVARLAGINITRTIYDGQQIRVFTCLLRLAGQKGFILPDTQGRFRGLDKEAPKRPAVPRGEGERPGDGNGDEDKDDDEDGDEDGDEREEVARETGGRHVGYQGARVLDPTSGFHVDPVVVFDFASLYPSIIQAHNLCFSTLSLRPEAVAHLEADRDYLEIEVGGRRLFFVKAHVRESLLSILLRDWLAMRKQIRSRIPQSTPEEAVLLDKQQAAIKVVCNSVYGFTGVQHGLLPCLHVAATVTTIGREMLLATRAYVHARWAEFDQLLADFPEAAGMRAPGPYSMRIIYGDTDSIFVLCRGLTAAGLVAMGDKMASHISRALFLPPIKLECEKTFTKLLLIAKKKYIGVICGGKMLIKGVDLVRKNNCAFINRTSRALVDLLFYDDTVSGAAAALAERPAEEWLARPLPEGLQAFGAVLVDAHRRITDPERDIQDFVLTAELSRHPRAYTNKRLAHLTVYYKLMARRAQVPSIKDRIPYVIVAQTREVEETVARLAALRELDAAAPGDEPAPPAALPSPAKRPRETPSHADPPGGASKPRKLLVSELAEDPGYAIARGVPLNTDYYFSHLLGAACVTFKALFGNNAKITESLLKRFIPETWHPPDDVAARLRAAGFGPAGAGATAEETRRMLHRAFDTLA

SEQ ID NO: 50 - protein, 1240 aa, Human alphaherpesvirus 2
  source 1..1240: note = HSV-2 strain 333
MFCAAGGPTSPGGKSAARAASGFFAPHNPRGATQTAPPPCRRQNFYNPHLAQTGTQPKAPGPAQRHTYYSECDEFRFIAPRSLDEDAPAEQRTGVHDGRLRRAPKVYCGGDERDVLRVGPEGFWPRRLRLWGGADHAPEGFDPTVTVFHVYDILEHVEHAYSMRAAQLHERFMDAITPAGTVITLLGLTPEGHRVAVHVYGTRQYFYMNKAEVDRHLQCRAPRDLCERLAAALRESPGASFRGISADHFEAEVVERADVYYYETRPTLYYRVFVRSGRALAYLCDNFCPAIRKYEGGVDATTRFILDNPGFVTFGWYRLKPGRGNAPAQPRPPTAFGTSSDVEFNCTADNLAVEGAMCDLPAYKLMCFDIECKAGGEDELAFPVAERPEDLVIQISCLLYDLSTTALEHILLFSLGSCDLPESHLSDLASRGLPAPVVLEFDSEFEMLLAFMTFVKQYGPEFVTGYNIINFDWPFVLTKLTEIYKVPLDGYGRMNGRGVFRVWDIGQSHFQKRSKIKVNGMVNIDMYGIITDKVKLSSYKLNAVAEAVLKDKKKDLSYRDIPAYYASGPAQRGVIGEYCVQDSLLVGQLFFKFLPHLELSAVARLAGINITRTIYDGQQIRVFTCLLRLAGQKGFILPDTQGRFRGLDKEAPKRPAVPRGEGERPGDGNGDEDKDDDEDGDEDGDEREEVARETGGRHVGYQGARVLDPTSGFHVDPVVVFDFASLYPSIIQAHNLCFSTLSLRPEAVAHLEADRDYLEIEVGGRRLFFVKAHVRESLLSILLRDWLAMRKQIRSRIPQSTPEEAVLLDKQQAAIKVVCNSVYGFTGVQHGLLPCLHVAATVTTIGREMLLATRAYVHARWAEFDQLLADFPEAAGMRAPGPYSMRIIYGDTDSIFVLCRGLTAAGLVAMGDKMASHISRALFLPPIKLECEKTFTKLLLIAKKKYIGVICGGKMLIKGVDLVRKNNCAFINRTSRALVDLLFYDDTVSGAAAALAERPAEEWLARPLPEGLQAFGAVLVDAHRRITDPERDIQDFVLTAELSRHPRAYTNKRLAHLTVYYKLMARRAQVPSIKDRIPYVIVAQTREVEETVARLAALRELDAAAPGDEPAPPAALPSPAKRPRETPSHADPPGGASKPRKLLVSELAEDPGYAIARGVPLNTDYYFSHLLGAACVTFKALFGNNAKITESLLKRFIPETWHPPDDVAARLRAAGFGPAGAGATAEETRRMLHRAFDTLA

SEQ ID NO: 51 - protein, 1238 aa, Human alphaherpesvirus 2
  source 1..1238: note = HSV-2 strain MS
MFCAAGGPTSPGGKSAARAASGFFAPHNPRGATQTAPPPCRRQNFYNPHLAQTGTQPKAPGPAQRHTYYSECDEFRFIAPRSLDEDAPAEQRTGVHDGRLRRAPKVYCGGDERDVLRVGPEGFWPRRLRLWGGADHAPKGFDPTVTVFHVYDILEHVEHAYSMRAAQLHERFMDAITPAGTVITLLGLTPEGHRVAVHVYGTRQYFYMNKAEVDRHLQCRAPRDLCERLAAALRESPGASFRGISADHFEAEVVERADVYYYETRPTLYYRVFVRSGRALAYLCDNFCPAIRKYEGGVDATTRFILDNPGFVTFGWYRLKPGRGNAPAQPRPPTAFGTSSDVEFNCTADNLAVEGAMCDLPAYKLMCFDIECKAGGEDELAFPVAERPEDLVIQISCLLYDLSTTALEHILLFSLGSCDLPESHLSDLASRGLPAPVVLEFDSEFEMLLAFMTFVKQYGPEFVTGYNIINFDWPFVLTKLTEIYKVPLDGYGRMNGRGVFRVWDIGQSHFQKRSKIKVNGMVNIDMYGIITDKVKLSSYKLNAVAEAVLKDKKKDLSYRDIPAYYASGPAQRGVIGEYCVQDSLLVGQLFFKFLPHLELSAVARLAGINITRTIYDGQQIRVFTCLLRLAGQKGFILPDTQGRFRGLDKEAPKRPAVPRGEGERPGDGNGDEDKDDDEDEDGDEREEVARETGGRHVGYQGARVLDPTSGFHVDPVVVFDFASLYPSIIQAHNLCFSTLSLRPEAVAHLEADRDYLEIEVGGRRLFFVKAHVRESLLSILLRDWLAMRKQIRSRIPQSTPEEAVLLDKQQAAIKVVCNSVYGFTGVQHGLLPCLHVAATVTTIGREMLLATRAYVHARWAEFDQLLADFPEAAGMRAPGPYSMRIIYGDTDSIFVLCRGLTAAGLVAMGDKMASHISRALFLPPIKLECEKTFTKLLLIAKKKYIGVICGGKMLIKGVDLVRKNNCAFINRTSRALVDLLFYDDTVSGAAAALAERPAEEWLARPLPEGLQAFGAVLVDAHRRITDPERDIQDFVLTAELSRHPRAYTNKRLAHLTVYYKLMARRAQVPSIKDRIPYVIVAQTREVEETVARLAALRELDAAAPGDEPAPPAALPSPAKRPRETPSHADPPGGASKPRKLLVSELAEDPGYAIARGVPLNTDYYFSHLLGAACVTFKALFGNNAKITESLLKRFIPETWHPPDDVAARLRAAGFGPAGAGATAEETRRMLHRAFDTLA

SEQ ID NO: 52 - protein, 1066 aa, Human alphaherpesvirus 2
  source 1..1066: note = HSV-2 strain HG52
MGTEDCDHEGRSVAAPVEVTALYATDGCVITSSLALLTNCLLGAEPLYIFSYDAYRSDAPNGPTGAPTEQERFEGSRALYRDAGGLNGDSFRVTFCLLGTEVGVTHHPKGRTRPMFVCRFERADDVAVLQDALGRGTPLLPAHVTATLDLEATFALHANIIMALTVAIVHNAPARIGSGSTAPLYEPGESMRSVVGRMSLGQRGLTTLFVHHEARVLGAYRRAYYGSAQSPFWFLSKFGPDEKSLVLAARYYLLQAPRLGGAGATYDLQAVKDICATYAIPHDPRPDTLSAASLTSFAAITRFCCTSQYSRGAAAAGFPLYVERRIAADVRETGALEKFIAHDRSCLRVSDREFITYIYLAHFECFSPPRLATHLRAVTTHDPSPAASTEQPSPLGREAVEQFFRHVRAQLNIREYVKQNVTPRETALAGDAAAAYLRARTYAPAALTPAPAYCGVADSSTKMMGRLAEAERLLVPHGWPAFAPTTPGDDAGGGTAAPQTCGIVKRLLKLAATEQQGTTPPAIAALMQDASVQTPLPVYRITMSPTGQAFAAAARDDWARVTRDARPPEATVVADAAAAPEPGALGRRLTRRICARGPALPPGGLAVGGQMYVNRNEIFNAALAVTNIILDLDIALKEPVPFPRLHEALGHFRRGALAAVQLLFPAARVDPDAYPCYFFKSACRPRAPPVCAGDGPSAGGDDGDGDWFPDAGGPGDEEWEEDTDPMDTTHGPLPDDEAAYLDLLHEQIPAATPSEPDSVVCSCADKIGLRVCLPVPAPYVVHGSLTMRGVARVIQQAVLLDRDFVEAVGSHVKNFLLIDTGVYAHGHSLRLPYFAKIGPDGSACGRLLPVFVIPPACEDVPAFVAAHADPRRFHFHAPPMFSAAPREIRVLHSLGGDYVSFFEKKASRNALEHFGRRETLTEVLGRYDVRPDAGETVEGFASELLGRIVACIEAHFPEHAREYQAVSVRRAVIKDDWVLLQLIPGRGALNQSLSCLRFKHGRASRATARTFLALSVGTNNRLCASLCQQCFATKCDNNRLHTLFTVDAGTPCSRSAPSSTSRPSSS

SEQ ID NO: 53 - protein, 1067 aa, Human alphaherpesvirus 2
  source 1..1067: note = HSV-2 strain 333
MGTEDCDHEGRSVAAPVEVTALYATDGCVITSSLALLTNCLLGAEPLYIFSYDAYRPDAPNGPTGAPTEQERFEGSRALYRDAGGLNGDSFRVTFCLLGTEVGVTHHPKGRTRPMFVCRFERADDVAVLQDALGRGTPLLPAHITATLDLEATFALHANIIMALTVAIVHNAPARIGSGSTAPLYEPGESMRSVVGRMSLGQRGLTTLFVHHEARVLAAYRRAYYGSAQSPFWFLSKFGPDEKSLVLAARYYLLQAPRLGGAGATYDLQAVKDICATYAIPHDPRPDTLSAASLTSFAAITRFCCTSQYSRGAAAAGFPLYVERRIAADVRETGALEKFIAHDRSCLRVSDREFITYIYLAHFECFSPPRLATHLRAVTTHDPSPAASTEQPSPLGREAVEQFFRHVRAQLNIREYVKQNVTPRETALAGDAAAAYLRARTYAPAALTPAPAYCGVADSSTKMMGRLAEAERLLVPHGWPAFAPTTPGDDAGGGTAAPQTCGIVKRLLKLAATEQQGTTPPAIAALMQDASVQTPLPVYRITMSPTGQAFAAAARDDWARVTRDARPPEATVVADAAAAPEPGALGRRLTRRICARGPALPPGGLAVGGQMYVNRNEIFNAALAVTNIILDLDIALKEPVPFPRLHEALGHFRRGALAAVQLLFPAARVDPDAYPCYFFKSACRPRAPPVCAGDGPSAGGDDGDGDWFPDAGGDDGDEEWEEDTDPMDTTHGPLPDDEAAYLDLLHEQIPAATPSEPDSVVCSCADKIGLRVCLPVPAPYVVHGSLTMRGVARVIQQAVLLDRDFVEAVGSHVKNFLLIDTGVYAHGHSLRLPYFAKIGPDGSACGRLLPVFVIPPACEDVPAFVAAHADPRRFHFHAPPMFSAAPREIRVLHSLGGDYVSFFEKKASRNALEHFGRRETLTEVLGRYDVRPDAGETVEGFASELLGRIVACIEAHFPEHAREYQAVSVRRAVIKDDWVLLQLIPGRGALNQSLSCLRFKHGRASRATARTFLALSVGTNNRLCASLCQQCFATKCDNNRLHTLFTVDAGTPCSRSAPSSTSRPSSS

SEQ ID NO: 54 - protein, 1066 aa, Human alphaherpesvirus 2
  source 1..1066: note = HSV-2 strain MS
MGTEDCDHEGRSVAAPVEVTALYATDGCVITSSLALLTNCLLGAEPLYIFSYDAYRPDAPNGPTGAPTEQERFEGSRALYRDAGGLNGDSFRVTFCLLGTEVGVTHHPKGRTRPMFVCRFERADDVAVLQDALGRGTPLLPAHITATLDLEATFALHANIIMALTVAIVHNAPARIGSGSTAPLYEPGESMRSVVGRMSLGQRGLTTLFVHHEARVLAAYRRAYYGSAQSPFWFLSKFGPDEKSLVLAARYYLLQAPRLGGAGATYDLQAVKDICATYAIPHDPRPDTLSAASLTSFAAITRFCCTSQYSRGAAAAGFPLYVERRIAADVRETSALEKFIAHDRSCLRVSDREFITYIYLAHFECFSPPRLATHLRAVTTHDPSPAASTEQPSPLGREAVEQFFRHVRAQLNIREYVKQNVTPRETALAGDAAAAYLRARTYAPAALTPAPAYCGVADSSTKMMGRLAEAERLLVPHGWPAFAPTTPGDDAGGGTAAPQTCGIVKRLLKLAATEQQGTTPPAIAALMQDASVQTPLPVYRITMSPTGQAFAAAARDDWARVTRDARPPEATVVADAAAAPEPGALGRRLTRRICARGPALPPGGLAVGGQMYVNRNEIFNAALAVTNIILDLDIALKEPVPFPRLHEALGHFRRGALAAVQLLFPAARVDPDAYPCYFFKSACRPRAPPVCAGDGPSAGGDDGDGDWFPDAGGPGDEEWEEDTDPMDTTHGPLPDDEAAYLDLLHEQIPAATPSEPDSVVCSCADKIGLRVCLPVPAPYVVHGSLTMRGVARVIQQAVLLDRDFVEAVGSHVKNFLLIDTGVYAHGHSLRLPYFAKIGPDGSACGRLLPVFVIPPACEDVPAFVAAHADPRRFHFHAPPMFSAAPREIRVLHSLGGDYVSFFEKKASRNALEHFGRRETLTEVLGRYDVRPDAGETVEGFASELLGRIVACIEAHFPEHAREYQAVSVRRAVIKDDWVLLQLIPGRGALNQSLSCLRFKHGRASRATARTFLALSVGTNNRLCASLCQQCFATKCDNNRLHTLFTVDAGTPCSRSAPSSTSRPSSS

SEQ ID NO: 55 - protein, 512 aa, Human alphaherpesvirus 2
  source 1..512: note = HSV-2 strain HG52
MATDIDMLIDLGLDLSDSELEEDALERDEEGRRDDPESDSSGECSSSDEDMEDPCGDGGAEAIDAAIPKGPPARPEDAGTPEASTPRPAARRGADDPPPATTGVWSRLGTRRSASPREPHGGKVARIQPPSTKAPHPRGGRRGRRRGRGRYGPGGADSTPKPRRRVSRNAHNQGGRHPASARTDGPGATHGEARRGGEQLDVSGGPRPRGTRQAPPPLMALSLTPPHADGRAPVPERKAPSADTIDPAVRAVLRSISERAAVERISESFGRSALVMQDPFGGMPFPAANSPWAPVLATQAGGFDAETRRVSWETLVAHGPSLYRTFAANPRAASTAKAMRDCVLRQENLIEALASADETLAWCKMCIHHNLPLRPQDPIIGTAAAVLENLATRLRPFLQCYLKARGLCGLDDLCSRRRLSDIKDIASFVLVILARLANRVERGVSEIDYTTVGVGAGETMHFYIPGACMAGLIEILDTHRQECSSRVCELTASHTIAPLYVHGKYFYCNSLF

SEQ ID NO: 56 - protein, 512 aa, Human alphaherpesvirus 2
  source 1..512: note = HSV-2 strain 333
MATDIDMLIDLGLDLSDSELEEDALERDEEGRRDDPESDSSGECSSSDEDMEDPCGDGGAEAIDAAIPKGPPARPEDAGTPEASTPRPAARRGADDPPPATTGVWSRLGTRRSASPREPHGGKVARIQPPSTKAPHPRGGRRGRRRGRGRYGPGGADSTPNPRRRVSRNAHNQGGRHPASARTDGPGATHGEARRGGEQLDVSGGPRPRGTRQAPPPLMALSLTPPHADGRAPVPERKAPSADTIDPAVRAVLRSISERAAVERISESFGRSALVMQDPFGGMPFPAANSPWAPVLATQAGGFDAETRRVSWETLVAHGPSLYRTFAANPRAASTAKAMRDCVLRQENLIEALASADETLAWCKMCIHHNLPLRPQDPIIGTAAAVLENLATRLRPFLQCYLKARGLCGLDDLCSRRRLSDIKDIASFVLVILARLANRVERGVSEIDYTTVGVGAGETMHFYIPGACMAGLIEILDTHRQECSSRVCELTASHTIAPLYVHGKYFYCNSLF

SEQ ID NO: 57 - protein, 512 aa, Human alphaherpesvirus 2; identical to SEQ ID NO: 56
  source 1..512: note = HSV-2 strain MS

SEQ ID NO: 58 - protein, 224 aa, Human alphaherpesvirus 2; identical to SEQ ID NO: 1
  source 1..224: note = HSV-2 strain HG52

SEQ ID NO: 59 - protein, 224 aa, Human alphaherpesvirus 2; identical to SEQ ID NO: 1
  source 1..224: note = HSV-2 strain 333

SEQ ID NO: 60 - protein, 224 aa, Human alphaherpesvirus 2; identical to SEQ ID NO: 1
  source 1..224: note = HSV-2 strain MS

SEQ ID NO: 61 - protein, 224 aa, Human alphaherpesvirus 2; identical to SEQ ID NO: 1
  source 1..224: note = HSV-2 strain G

SEQ ID NO: 62 - protein, 230 aa, Human alphaherpesvirus 2
  source 1..230: note = HSV-2 strain HG52
GPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTPPSSPRDPTPAPGDTGTPAPASGEIAPPNSTRSASESRHR

SEQ ID NO: 63 - protein, 372 aa, Human alphaherpesvirus 2
  source 1..372: note = HSV-2 strain 333
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGSDYGSCDPAQLPFSAPRLGPSSVYTPGASRPTPPRTTTSPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 64 - protein, 372 aa, Human alphaherpesvirus 2
  source 1..372: note = HSV-2 strain MS
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTSPSSPRDPTPAPGDTGTPAPASGERAPPNSTRSASESRHRLTVAQVIQIAIPASIIAFVFLGSCICFIHRCRRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 65 - protein, 372 aa, Human alphaherpesvirus 2
  source 1..372: note = HSV-2 strain G
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTPPSSPRDPTPAPGDTGTPAPASGESAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 66 - protein, 545 aa, Human alphaherpesvirus 2
  source 1..545: note = HSV-2 strain HG52
MARGAGLVFFVGVWVVSCLAAAPRTSWKRVTSGEDVVLLPAPAERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYPSVLW

SEQ ID NO: 67 - protein, 382 aa, Human alphaherpesvirus 2
  source 1..382: note = HSV-2 strain 333
RTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRA

SEQ ID NO: 68 - protein, 545 aa, Human alphaherpesvirus 2
  source 1..545: note = HSV-2 strain MS
MARGAGLVFFVGVWVVSCLAAAPRTSWKRVTSGEDVVLLPAPAERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 69 - protein, 548 aa, Human alphaherpesvirus 2
  source 1..548: note = HSV-2 strain G
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHMRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 70 - protein, 838 aa, Human alphaherpesvirus 2
  source 1..838: note = HSV-2 strain HG52
MGPGLWVVMGVLVGVAGGHDTYWTEQIDPWFLHGLGLARTYWRDTNTGRLWLPNTPDASDPQRGRLAPPGELNLTTASVPMLRWYAERFCFVLVTTAEFPRDPGQLLYIPKTYLLGRPRNASLPELPEAGPTSRPPAEVTQLKGLSHNPGASALLRSRAWVTFAAAPDREGLTFPRGDDGATERHPDGRRNAPPPGPPAGTPRHPTTNLSIAHLHNASVTWLAARGLLRTPGRYVYLSPSASTWPVGVWTTGGLAFGCDAALVRARYGKGFMGLVISMRDSPPAEIIVVPADKTLARVGNPTDENAPAVLPGPPAGPRYRVFVLGAPTPADNGSALDALRRVAGYPEESTNYAQYMSRAYAEFLGEDPGSGTDARPSLFWRLAGLLASSGFAFVNAAHAHDAIRLSDLLGFLAHSRVLAGLAARGAAGCAADSVFLNVSVLDPAARLRLEARLGHLVAAILEREQSLAAHALGYQLAFVLDSPAAYGAVAPSAARLIDALYAEFLGGRALTAPMVRRALFYATAVLRAPFLAGAPSAEQRERARRGLLITTALCTSDVAAATHADLRAALARTDHQKNLFWLPDHFSPCAASLRFDLAEGGFILDALAMATRSDIPADVMAQQTRGVASVLTRWAHYNALIRAFVPEATHQCSGPSHNAEPRILVPITHNASYVVTHTPLPRGIGYKLTGVDVRRPLFITYLTATCEGHAREIEPKRLVRTENRRDLGLVGAVFLRYTPAGEVMSVLLVDTDATQQQLAQGPVAGTPNVFSSDVPSVALLLFPNGTVIHLLAFDTLPIATIAPGFLAASALGVVMITAALAGILRVVRTCVPFLWRRE

SEQ ID NO: 71 - protein, 838 aa, Human alphaherpesvirus 2; identical to SEQ ID NO: 70
  source 1..838: note = HSV-2 strain 333

SEQ ID NO: 72 - protein, 838 aa, Human alphaherpesvirus 2
  source 1..838: note = HSV-2 strain MS
MGPGLWVVMGVLVGVAGGHDTYWTEQIDPWFLHGLGLARTYWRDTNTGRLWLPNTPDASDPQRGRLAPPGELNLTTASVPMLRWYAERFCFVLVTTAEFPRDPGQLLYIPKTYLLGRPRNASLPELPEAGPTSRPPAEVTQLKGLSHNPGASALLRSRAWVTFAAAPDREGLTFPRGDDGATERHPDGRRNAPPPGPPAGAPRHPTTNLSIAHLHNASVTWLAARGLLRTPGRYVYLSPSASTWPVGVWTTGGLAFGCDAALVRARYGKGFMGLVISMRDSPPAEIIVVPADKTLARVGNPTDENAPAVLPGPPAGPRYRVFVLGAPTPADNGSALDALRRVAGYPEESTNYAQYMSRAYAEFLGEDPGSGTDARPSLFWRLAGLLASSGFAFVNAAHAHDAIRLSDLLGFLTHSRVLAGLAARGAAGCAADSVFLNVSVLDPAARLRLEARLGHLVAAILEREQSLAAHALGYQLAFVLDSPAAYGAVAPSAARLIDALYAEFLGGRALTAPMVRRALFYATAVLRAPFLAGAPSAEQRERARRGLLITTALCTSDVAAATHADLRAALARTDHQKNLFWLPDHFSPCAASLRFDLAEGGFILDALAMATRSDIPADVMAQQTRGVASALTRWAHYNALIRAFVPEATHQCSGPSHNAEPRILVPITHNASYVVTHTPLPRGIGYKLTGVDVRRPLFITYLTATCEGHAREIEPKRLVRTENRRDLGLVGAVFLRYTPAGEVMSVLLVDTDATQQQLAQGPVAGTPNVFSSDVPSVALLLFPNGTVIHLLAFDTLPIATIAPGFLAASALGVVMITAALAGILRVVRTCVPFLWRRE

SEQ ID NO: 73 - protein, 838 aa, Human alphaherpesvirus 2; identical to SEQ ID NO: 70
  source 1..838: note = HSV-2 strain G

SEQ ID NO: 74 - protein, 901 aa, Human alphaherpesvirus 2
  source 1..901: note = HSV-2 strain G
MRGGGLICALVVGALVAAVASAAPAAPRASGGVAATVAANGGPASRPPPVPSPATTRARKRKTKKPPERPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 75 - RNA, 696 nt, synthetic construct
  source 1..696: mol_type = other RNA
ggccccacagtgtccctggtgtccgattctcttgtggatgctggcgctgtgggccctcagggattcgtggaagaagatctgagagtctttggcgagctgcacttcgtgggagcccaggtgccacacaccaactactacgacggcatcatcgagctgtttcactaccctctgggcaatcactgccccagagtggtgcacgtggtcacccttacagcctgtcctagaaggcctgccgtggccttcacactgtgcagatctacacaccacgctcacagccccgcctatcctacactggaactgggacttgccagacagcccctgctgagagtcagaaccgccaccagagattacgccggcctgtacgttctgagagtgtgggtcggaagcgccaccaacgccagcagatttgtgctgggagttgccctgagcgccaacggcacctttgtgtacaacggcagcgattacggcagctgcgatcctgctcagctgccttttagcgcccctagactgggccctagcagcgtttacacacctggcgcctctagacccacacctcctagaacaacaacccctcctagcagccccagagatcccacaccagctccaggcgatactggaactcctgctcctgcctctggcgagatcgcccctcctaattctacaagaagcgccagcgagagccggcacagatgataa

SEQ ID NO: 76 - RNA, 696 nt, synthetic construct; identical to SEQ ID NO: 75
  source 1..696: mol_type = other RNA

SEQ ID NO: 77 - RNA, 696 nt, synthetic construct
  source 1..696: mol_type = other RNA
ggtcctaccgtgtctctcgtgtctgacagcctggtagacgccggtgctgtgggccctcagggcttcgttgaggaggatctgcgggtgtttggggaattgcactttgtgggcgctcaggttccgcacaccaactactacgatgggatcatcgagctgttccactatccccttgggaaccattgcccgagagtcgtgcatgtggtcaccctgaccgcttgccctcgccgaccagcagtcgcctttaccctgtgtcgcagcactcaccatgcccatagtccagcatatcccactctggaactgggactggccaggcaaccactgctgagagtgcgcacagccacacgggactatgccggactgtatgtgctcagagtctgggttgggagcgcaaccaatgcgtcacgttttgtgctcggagtagccctctccgccaatggtacgttcgtctacaacggctccgattacggcagctgcgatccagcgcagctgccattctcagcacccaggctcggtccctcaagcgtctacacaccaggcgcttctagacccactccacccaggactacgacacctccctccagtcctcgtgaccccacaccggctcctggagacaccggaactcccgctcctgcgtctggcgagattgcacctcccaattccacacggagtgccagcgaatccaggcaccgctgataa

SEQ ID NO: 78 - RNA, 696 nt, synthetic construct
  source 1..696: mol_type = other RNA
ggaccaacagtgtctctggtgtctgattctctggtggacgctggagctgtgggacctcagggatttgtggaagaagatctgagagtgtttggagaactgcactttgtgggagctcaggtgcctcacacaaattattatgacggaatcatcgaactgtttcactaccctctgggaaatcactgcccaagagtggtgcatgtggtgacactgacagcttgcccaagaagacctgctgtggcttttacactgtgcagatcaacacaccacgctcactctccagcttacccaacactggaactgggtctggcaagacagcctctgctgagagtgagaacagcaacaagagattatgctggactgtatgtgctgagagtgtgggtgggatctgcaacaaatgcttcaagatttgtgctgggagtggctctgtctgcaaatggaacatttgtgtacaatggatctgattatggatcttgtgatcctgctcagctgcctttttctgctccaagactgggaccttcttctgtgtacacacctggagcttcaagaccaacaccaccaagaacaacaacacctccttcttctccaagagatccaacacctgctcctggagacacaggaacacctgctcctgcttctggagaaattgctcctccaaattcaacgagatctgcttctgaatcaagacacagatgataa

SEQ ID NO: 79 - RNA, 696 nt, synthetic construct; identical to SEQ ID NO: 78
  source 1..696: mol_type = other RNA

SEQ ID NO: 80 - RNA, 1149 nt, Human alphaherpesvirus 2
  source 1..1149: mol_type = genomic RNA
cgcacctcctggaagcgcgtgacctccggcgaggacgtggtgctgctgcccgcccccgccggccccgaggagcgcacccgcgcccacaagctgctgtgggccgccgagcccctggacgcctgcggccccctgcgcccctcctgggtggccctgtggcccccccgccgcgtgctggagaccgtggtggacgccgcctgcatgcgcgcccccgagcccctggccatcgcctactccccccccttccccgccggcgacgagggcctgtactccgagctggcctggcgcgaccgcgtggccgtggtgaacgagtccctggtgatctacggcgccctggagaccgactccggcctgtacaccctgtccgtggtgggcctgtccgacgaggcccgccaggtggcctccgtggtgctggtggtggagcccgcccccgtgcccacccccacccccgacgactacgacgaggaggacgacgccggcgtgtccgagcgcacccccgtgtccgtgcccccccccacccccccccgccgcccccccgtggccccccccacccacccccgcgtgatccccgaggtgtcccacgtgcgcggcgtgaccgtgcacatggagacccccgaggccatcctgttcgcccccggcgagaccttcggcaccaacgtgtccatccacgccatcgcccacgacgacggcccctacgccatggacgtggtgtggatgcgcttcgacgtgccctcctcctgcgccgagatgcgcatctacgaggcctgcctgtaccacccccagctgcccgagtgcctgtcccccgccgacgccccctgcgccgtgtcctcctgggcctaccgcctggccgtgcgctcctacgccggctgctcccgcaccaccccccccccccgctgcttcgccgaggcccgcatggagcccgtgcccggcctggcctggctggcctccaccgtgaacctggagttccagcacgcctccccccagcacgccggcctgtacctgtgcgtggtgtacgtggacgaccacatccacgcctggggccacatgaccatctccaccgccgcccagtaccgcaacgccgtggtggagcagcacctgccccagcgccagcccgagcccgtggagcccacccgcccccacgtgcgcgcctaa

SEQ ID NO: 81 - RNA, 1152 nt, synthetic construct
  source 1..1152: mol_type = other RNA
agcagaaccagctggaaaagagtgaccagcggcgaggatgtggtgctgcttcctgctcctgctggccccgaggaaagaacaagagcccacaaactgctgtgggccgctgagcctcttgatgcctgtggacctctcagacctagctgggttgcactgtggccacctcggagagtgctggaaacagtggtggatgccgcctgcatgagagcccctgaacctctggccattgcctactctccaccatttccagccggcgacgagggcctgtattctgagcttgcttggagagacagagtggccgtggtcaacgagagcctggttatctatggcgccctggaaaccgacagcggcctgtacacactgtctgtcgtgggcctgtctgacgaggctagacaggtggcatctgtggtcctggtggtggaacctgctccagtgcctacacctacacctgacgactacgacgaggaagatgacgctggcgtcagcgagagaacccctgtttctgtgcctcctcctacgcctcctcgtagacctcctgttgctcctccaacacaccccagagtgatccctgaagtgtctcacgtgcggggcgtgaccgtgcacatggaaacacctgaggccatcctgttcgcccctggcgagacatttggcaccaacgtgtccatccacgctatcgcccacgacgatggcccttacgccatggatgtcgtgtggatgagattcgacgtgcccagcagctgtgccgagatgagaatctatgaggcctgcctgtatcaccctcagctgcccgaatgtctgagccctgctgatgccccttgtgccgttagcagctgggcctatagactggccgtgcggtcttatgccggctgctctagaacaacccctcctcctcggtgtttcgccgaggccagaatggaacctgttcctggactggcctggctggcctccacagtgaacctggaatttcagcacgcctctccacagcacgccggcctgtatctgtgtgtggtgtacgtggacgatcacatccacgcctggggccacatgaccatctctacagccgctcagtaccggaacgccgtggttgaacagcatctgcctcagagacagcccgagcctgtggaacctacaagacctcatgttcgggcctga

SEQ ID NO: 82 - RNA, 1149 nt, synthetic construct
  source 1..1149: mol_type = other RNA
cgcacctcttggaaacgcgttacttccggggaggacgttgtcctccttccagcacccgcaggacctgaagagaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaagaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 83 - RNA, 1158 nt, synthetic construct
  source 1..1158: mol_type = other RNA
aaatatgctcgcacctcttggaaacgcgttacttccggggaggacgttgtcctccttccagcacccgcaggacctgaagagaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaagaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 84 - RNA, 1149 nt, synthetic construct
  source 1..1149: mol_type = other RNA
agaacctcctggaaaagagtgacctccggcgaagatgtggtgctgctgcccgcccccgccggccccgaagaaagaaccagagcccacaaactgctgtgggccgccgaacccctggatgcctgcggccccctcagaccctcctgggtggccctgtggccccctagaagggtgctggaaaccgtggtggatgccgcctgcatgagagcccccgaacccctggccatcgcctactcccctcccttccccgccggcgatgaaggcctgtactccgaactggcctggagagatagagtggccgtggtgaacgaatccctggtgatctacggcgccctggaaaccgattccggcctgtacaccctgtccgtggtgggcctgtccgatgaagccagacaggtggcctccgtggtgctggtggtggaacccgcccccgtgcccacccccacccccgatgattacgatgaagaagatgatgccggcgtgtccgaaagaacccccgtgtccgtgccccctcccacccctccccgcagaccccctgtggcccctcccacccaccccagagtgatccccgaagtgtcccacgtgagaggcgtgaccgtgcacatggaaacccccgaagccatcctgttcgcccccggcgaaaccttcggcaccaacgtgtccatccacgccatcgcccacgatgatggcccctacgccatggatgtggtgtggatgagattcgatgtgccctcctcctgcgccgaaatgagaatctacgaagcctgcctgtaccacccccagctgcccgaatgcctgtcccccgccgatgccccctgcgccgtgtcctcctgggcctacagactggccgtgagatcctacgccggctgctccagaaccacccctccccctagatgcttcgccgaagccagaatggaacccgtgcccggcctggcctggctggcctccaccgtgaacctggaattccagcacgccagcccccagcacgccggcctgtacctgtgcgtggtgtacgtggatgatcacatccacgcctggggccacatgaccatctccaccgccgcccagtacagaaacgccgtggtggaacagcacctgccccagagacagcccgaacccgtggaacccaccagaccccacgtgagagcctga

SEQ ID NO: 85 - protein, 27 aa, Human alphaherpesvirus 1
MGGAAARLGAVILFVVIVGLHGVRSKY

SEQ ID NO: 86 - protein, 25 aa, Human alphaherpesvirus 1
MGGAAARLGAVILFVVIVGLHGVRG

SEQ ID NO: 87 - protein, 25 aa, Human alphaherpesvirus 2
MGRLTSGVGTAALLVVAVGLRVVCA

SEQ ID NO: 88 - protein, 28 aa, Human alphaherpesvirus 2
MGRLTSGVGTAALLVVAVGLRVVCAKYA

SEQ ID NO: 89 - protein, 18 aa, unidentified
  source 1..18: note = Plasmodium falciparum (isolate 3D7)
MMRKLAILSVSSFLFVEA

SEQ ID NO: 90 - protein, 19 aa, Severe acute respiratory syndrome-related coronavirus
  source 1..19: note = SARS-CoV-2-S
MFVFLVLLPLVSSQCVNLT

SEQ ID NO: 91 - protein, 21 aa, Homo sapiens
MDWIWRILFLVGAATGAHSQM

SEQ ID NO: 92 - protein, 20 aa, Homo sapiens
METPAQLLFLLLLWLPDTTG

SEQ ID NO: 93 - protein, 18 aa, synthetic construct
MDWTWILFLVAAATRVHS

SEQ ID NO: 94 - protein, 24 aa, Japanese encephalitis virus
MLGSNSGQRVVFTILLLLVAPAYS

SEQ ID NO: 95 - protein, 17 aa, unidentified
  source 1..17: note = Vesicular stomatitis virus G
MKCLLYLAFLFIGVNCA

SEQ ID NO: 96 - protein, 24 aa, synthetic construct
MCRGLSAVLILLVSLSAQLHVVVG

SEQ ID NO: 97 - protein, 19 aa, Homo sapiens
MELGLSWIFLLAILKGVQC

SEQ ID NO: 98 - protein, 19 aa, Homo sapiens
MELGLRWVFLVAILEGVQC

SEQ ID NO: 99 - protein, 19 aa, Homo sapiens
MKHLWFFLLLVAAPRWVLS

SEQ ID NO: 100 - protein, 19 aa, Homo sapiens
MDWTWRILFLVAAATGAHS

SEQ ID NO: 101 - protein, 19 aa, Homo sapiens
MDWTWRFLFVVAAATGVQS

SEQ ID NO: 102 - protein, 19 aa, Homo sapiens
MEFGLSWLFLVAILKGVQC

SEQ ID NO: 103 - protein, 19 aa, Homo sapiens
MEFGLSWVFLVALFRGVQC

SEQ ID NO: 104 - protein, 26 aa, Homo sapiens
MDLLHKNMKHLWFFLLLVAAPRWVLS

SEQ ID NO: 105 - protein, 22 aa, Homo sapiens
MDMRVPAQLLGLLLLWLSGARC

SEQ ID NO: 106 - protein, 22 aa, Homo sapiens
MKYLLPTAAAGLLLLAAQPAMA

SEQ ID NO: 107 - protein, 24 aa, Human alphaherpesvirus 2
MPGRSLQGLAILGLWVCATGLVVR

SEQ ID NO: 108 - protein, 21 aa, Human alphaherpesvirus 2
MPGRSLQGLAILGLWVCATGL

SEQ ID NO: 109 - protein, 27 aa, Human alphaherpesvirus 1
MGGAAARLGAVILFVVIVGLHGVRGKY

SEQ ID NO: 110 - protein, 29 aa, Human alphaherpesvirus 2
MGRLTSGVGTAALLVVAVGLRVVCAKYAL

SEQ ID NO: 111 - protein, 30 aa, Human alphaherpesvirus 2
MGRLTSGVGTAALLVVAVGLRVVCAKYALA

SEQ ID NO: 112 - protein, 27 aa, Human alphaherpesvirus 2
MALGRVGLAVGLWGLLWVGVVVVLANA

SEQ ID NO: 113 - protein, 19 aa, synthetic construct
MRMQLLLLIALSLALVTNS

SEQ ID NO: 114 - protein, 32 aa, Human alphaherpesvirus 1
MHQGAPSWGRRWFVVWALLGLTLGVLVASAAP

SEQ ID NO: 115 - protein, 26 aa, Human alphaherpesvirus 2
MARGAGLVFFVGVWVVSCLAAAPRTS

SEQ ID NO: 116 - protein, 34 aa, unidentified
  source 1..34: note = Ebola virus
MGVTGILQLPRDRFKRTSFFLWVIILFQRTFSIP

SEQ ID NO: 117 - protein, 25 aa, Homo sapiens
MAISGVPVLGFFIIAVLMSAQESWA

SEQ ID NO: 118 - protein, 30 aa, synthetic construct
MDSKGSSQKGSRLLLLLVVSNLLLPQGVVG

SEQ ID NO: 119 - protein, 15 aa, Japanese encephalitis virus
MWLVSLAIVTACAGA

SEQ ID NO: 120 - protein, 15 aa, synthetic construct
MFVFLVLLPLVSSQC

SEQ ID NO: 121 - RNA, 81 nt, Human alphaherpesvirus 1
  source 1..81: mol_type = genomic RNA
atggggggggctgccgccaggttgggggccgtgattttgtttgtcgtcatagtgggcctccatggggtccgcagcaaatat

SEQ ID NO: 122 - RNA, 81 nt, synthetic construct
  source 1..81: mol_type = other RNA
atgggaggagccgccgccagactgggagccgtgatcctgttcgtggtgatcgtgggactgcatggagtgagaagcaagtac

SEQ ID NO: 123 - RNA, 57 nt, Severe acute respiratory syndrome-related coronavirus
  source 1..57: mol_type = other RNA; note = SARS-CoV-2
atgtttgtgtttcttgtgctgctgcctcttgtgtcttctcagtgtgtgaatttgaca

SEQ ID NO: 124 - RNA, 63 nt, Homo sapiens
  source 1..63: mol_type = other RNA
atggattggatttggagaatcctgttcctcgtgggagccgctacaggagcccactcccagatg

SEQ ID NO: 125 - RNA, 57 nt, Homo sapiens
  source 1..57: mol_type = other RNA
atggagttgggactgagctggattttccttttggctattttaaaaggtgtccagtgt

SEQ ID NO: 126 - RNA, 57 nt, Homo sapiens
  source 1..57: mol_type = other RNA
atggaactggggctccgctgggttttccttgttgctattttagaaggtgtccagtgt

SEQ ID NO: 127 - RNA, 57 nt, Homo sapiens
  source 1..57: mol_type = other RNA
atgaaacacctgtggttcttcctcctgctggtggcagctcccagatgggtcctgtcc

SEQ ID NO: 128 - RNA, 57 nt, Homo sapiens
  source 1..57: mol_type = other RNA
atggactggacctggaggatcctcttcttggtggcagcagcaacaggtgcccactcg

SEQ ID NO: 129 - RNA, 57 nt, Homo sapiens
  source 1..57: mol_type = other RNA
atggactggacctggaggttcctctttgtggtggcagcagctacaggtgtccagtcc

SEQ ID NO: 130 - RNA, 57 nt, Homo sapiens
  source 1..57: mol_type = other RNA
atggagtttgggctgagctggctttttcttgtggcgattctaaaaggtgtccagtgt

SEQ ID NO: 131 - RNA, 57 nt, Homo sapiens
  source 1..57: mol_type = other RNA
atggagtttgggctgagctgggttttcctcgttgctctttttagaggtgtccagtgt

SEQ ID NO: 132 - RNA, 78 nt, Homo sapiens
  source 1..78: mol_type = other RNA
atggacctcctgcacaagaacatgaaacacctgtggttcttcctcctcctggtggcagctcccagatgggtgctgtcc

SEQ ID NO: 133 - RNA, 66 nt, Homo sapiens
  source 1..66: mol_type = other RNA
atggacatgagggtccctgctcagctcctggggctcctgctgctctggctctcaggtgccagatgt

SEQ ID NO: 134 - RNA, 66 nt, Homo sapiens
  source 1..66: mol_type = other RNA
atgaaatacctattgcctacggcagccgctggattgttattactcgcggcccagccggccatggcc

SEQ ID NO: 135 - RNA, 72 nt, synthetic construct
  source 1..72: mol_type = other RNA
atgccaggacggagccttcagggcttggccatactggggctttgggtgtgtgcaaccgggttggtagttcga

SEQ ID NO: 136 - RNA, 72 nt, synthetic construct
  source 1..72: mol_type = other RNA
atgcctggcagatctctgcaaggactggccatcctcggactgtgggtttgcgcaacaggcctggttgttaga

SEQ ID NO: 137 - RNA, 72 nt, synthetic construct
  source 1..72: mol_type = other RNA
atgcctggaagatctctgcagggactggcaattctgggactgtgggtgtgtgcaacaggactggtggtgaga

SEQ ID NO: 138 - RNA, 63 nt, Human alphaherpesvirus 1
  source 1..63: mol_type = other RNA
atgggaggcgcagctgccagacttggtgctgtgatcctgttcgtggtgattgtagggctgcat

SEQ ID NO: 139 - RNA, 81 nt, Human alphaherpesvirus 1
  source 1..81: mol_type = other RNA
atgggaggcgcagctgccagacttggtgctgtgatcctgttcgtggtgattgtagggctgcatggtgtcaggggcaagtat

SEQ ID NO: 140 - RNA, 75 nt, Human alphaherpesvirus 2
  source 1..75: mol_type = genomic RNA
atgggccgcctgacctccggcgtgggcaccgccgccctgctggtggtggccgtgggcctgcgcgtggtgtgcgcc

SEQ ID NO: 141 - RNA, 75 nt, synthetic construct
  source 1..75: mol_type = other RNA
atgggcagactgacatctggcgtgggaacagctgctctgctggtggttgctgtgggcctgagagtcgtgtgtgcc

SEQ ID NO: 142 - RNA, 75 nt, synthetic construct
  source 1..75: mol_type = other RNA
atggggagactcacatcaggcgtaggaaccgctgccctgttggtcgtggccgttggtctgagagttgtgtgtgcc

SEQ ID NO: 143 - RNA, 75 nt, synthetic construct
  source 1..75: mol_type = other RNA
atgggcagactgacctccggcgtgggcaccgccgccctgctggtggtggccgtgggcctgagagtggtgtgcgcc

SEQ ID NO: 144 - RNA, 84 nt, Human alphaherpesvirus 2
  source 1..84: mol_type = genomic RNA
atgggccgcctgacctccggcgtgggcaccgccgccctgctggtggtggccgtgggcctgcgcgtggtgtgcgccaagtacgcc

SEQ ID NO: 145 - RNA, 75 nt, synthetic construct; identical to SEQ ID NO: 141
  source 1..75: mol_type = other RNA

SEQ ID NO: 146 - RNA, 84 nt, synthetic construct
  source 1..84: mol_type = other RNA
atggggagactcacatcaggcgtaggaaccgctgccctgttggtcgtggccgttggtctgagagttgtgtgtgccaaatatgct

SEQ ID NO: 147 - RNA, 75 nt, synthetic construct; identical to SEQ ID NO: 143
  source 1..75: mol_type = other RNA

SEQ ID NO: 148 - RNA, 87 nt, synthetic construct
  source 1..87: mol_type = other RNA
atgggcagactgacatctggcgtgggaacagctgctctgctggtggttgctgtgggcctgagagtcgtgtgtgccaaatacgccctg

SEQ ID NO: 149 - RNA, 87 nt, synthetic construct
  source 1..87: mol_type = other RNA
atggggagactcacatcaggcgtaggaaccgctgccctgttggtcgtggccgttggtctgagagttgtgtgtgccaaatatgctctg

SEQ ID NO: 150 - RNA, 87 nt, synthetic construct
  source 1..87: mol_type = other RNA
atgggcagactgacctccggcgtgggcaccgccgccctgctggtggtggccgtgggcctgagagtggtgtgcgccaaatacgccctg

SEQ ID NO: 151 - RNA, 81 nt, synthetic construct
  source 1..81: mol_type = other RNA
atggccttggggagagtgggccttgcagtgggtctgtggggactgctctgggttggcgtagtcgtcgtgctggctaacgca

SEQ ID NO: 152 - RNA, 57 nt, unidentified
  source 1..57: mol_type = genomic RNA
atgcgcatgcagctgctgctgctgatcgccctgtccctggccctggtgaccaactcc

SEQ ID NO: 153 - RNA, 54 nt, synthetic construct
  source 1..54: mol_type = other RNA
atgagaatgcagctgctgctcctgatcgccctgtctctggccctggtcaccaat

SEQ ID NO: 154 - RNA, 57 nt, synthetic construct
  source 1..57: mol_type = other RNA
atgcgcatgcaactgctcctgctgattgcgttgagccttgccctggtgaccaacagc

SEQ ID NO: 155 - RNA, 57 nt, synthetic construct
  source 1..57: mol_type = other RNA
atgagaatgcagctgctgctgctgatcgccctgtccctggccctggtgaccaactcc

SEQ ID NO: 156 - RNA, 63 nt, synthetic construct
  source 1..63: mol_type = other RNA
atgcctggcagatctctgcaaggactggccatcctcggactgtgggtttgcgcaacaggcctg

SEQ ID NO: 157 - RNA, 63 nt, synthetic construct
  source 1..63: mol_type = other RNA
atgccaggacggagccttcagggcttggccatactggggctttgggtgtgtgcaaccgggtct

SEQ ID NO: 158 - RNA, 63 nt, synthetic construct
  source 1..63: mol_type = other RNA
atgcctggaagatctctgcagggactggcaattctgggactgtgggtgtgtgcaacaggactg

SEQ ID NO: 159 - RNA, 78 nt, synthetic construct
  source 1..78: mol_type = other RNA
atggcacggggagccggattggtgttctttgtgggcgtgtgggtggtgagctgcttggcagccgcaccacgcacctct

SEQ ID NO: 160 - protein, 41 aa, Human alphaherpesvirus 1
GLIAGAVGGSLLAALVICGIVYWMRRHTQKAPKRIRLPHIR

SEQ ID NO: 161 - protein, 37 aa, Homo sapiens
PNKGSGTTSGTTRLLSGHTCFTLTGLLGTLVTMGLLT

SEQ ID NO: 162 - protein, 27 aa, synthetic construct
GYIPEAPRDGQAYVRKDGEWVLLSTFL

SEQ ID NO: 163 - protein, 11 aa, synthetic construct
SGGGGSGGGGS

SEQ ID NO: 164 - protein, 9 aa, synthetic construct
GSPGSGSGS

SEQ ID NO: 165 - protein, 10 aa, synthetic construct
GGSGGGGSGG

SEQ ID NO: 166 - protein, 10 aa, synthetic construct
GGSLGGGGSG

SEQ ID NO: 167 - protein, 10 aa, synthetic construct
GGGGSGGGGS

SEQ ID NO: 168 - skipped

SEQ ID NO: 169 - protein, 4 aa, synthetic construct
GGGS

SEQ ID NO: 170 - protein, 15 aa, synthetic construct
GGGGSGGGGSGGGGS

SEQ ID NO: 171 - protein, 10 aa, synthetic construct
AGNRVRRSVG

SEQ ID NO: 172 - skipped

SEQ ID NO: 173 - protein, 55 aa, synthetic construct
IVGIVAGLAVLAVVVIGAVVATVMCRRKSSGGKGGSYSQAASSDSAQGSDVSLTA

SEQ ID NO: 174 - protein, 96 aa, synthetic construct
CTDEIAPPLRCQSFPCLHPFCIPCMKTWIPLRNTCPLCNTPVAYLIVGVTASGSFSTIPIVNDPRTRVEAEAAVRAGTAVDFIWTGNPRTAPRSLS

SEQ ID NO: 175 - protein, 106 aa, synthetic construct
LPIAGVSSVVALAPYVNKTVTGDCLPVLDMETGHIGAYVVLVDQTGNVADLLRAAAPAWSRRTLLPEHARNCVRPPDYPTPPASEWNSLWMTPVGNMLFDQGTLVG

SEQ ID NO: 176 - protein, 236 aa, synthetic construct
RAAAWMRQVPDPEDVRVVILYSPLPGEDLAAGRAGGGPPPEWSAERGGLSCLLAALGNRLCGPATAAWAGNWTGAPDVSALGAQGVLLLSTRDLAFAGAVEFLGLLAGACDRRLIVVNAVRAADWPADGPVVSRQHAYLACEVLPAVQCAVRWPAARDLRRTVLASGRVFGPGVFARVEAAHARLYPDAPPLRLCRGANVRYRVRTRFGPDTLVPMSPREYRRAVLPALDGRAAAS

SEQ ID NO: 177 - protein, 200 aa, synthetic construct
ETLVAHGPSLYRTFAANPRAASTAKAMRDCVLRQENLIEALASADETLAWCKMCIHHNLPLRPQDPIIGTAAAVLENLATRLRPFLQCYLKARGLCGLDDLCSRRRLSDIKDIASFVLVILARLANRVERGVSEIDYTTVGVGAGETMHFYIPGACMAGLIEILDTHRQECSSRVCELTASHTIAPLYVHGKYFYCNSLF

SEQ ID NO: 178 - protein, 168 aa, synthetic construct
REDIETIAFIKRFSLDYGAINFINLAPNNVSELAMYYMANQILRYCDHSTYFINTLTAVIAGSRRPPSVQAAAAWAPQGGAGLEAGARALMDSLDAHPGAWTSMFASCNLLRPVMAARPMVVLGLSISKYYGMAGNDRVFQAGNWASLLGGKNACPLLIFDRTRKFVL

SEQ ID NO: 179 - protein, 170 aa, synthetic construct
RTFGSAPRLTEDDFGLLNYALAEMRRLCLDLPPVPPNAYTPYHLREYATRLVNGFKPLVRRSARLYRILGVLVHLRIRTREASFEEWMRSKEVDLDFGLTERLREHEAQLMILAQALNPYDCLIHSTPNTLVERGLQSALKYEEFYLKRFGGHYMESVFQMYTRIAGFLA

SEQ ID NO: 180 - protein, 84 aa, synthetic construct
KMTRGAPKASATPATDPARGRRPAQADSAVLLDAPAPTASGRTKTPAQGLAKKLHFSTAPPSPTAPWTPRVAGFNKRVFCAAVG

SEQ ID NO: 181 - protein, 245 aa, synthetic construct
LLNNYDVLVLDEVMSTLGQLYSPTMQQLGRVDALMLRLLRTCPRIIAMDATANAQLVDFLCSLRGEKNVHVVIGEYAMPGFSARRCLFLPRLGPEVLQAALRRRGPAGGAPPPDAPPDATFFGELEARLAGGDNVCIFSSTVSFAEVVARFCRQFTDRVLLLHSLTPPGDVTTWGRYRVVIYTTVVTVGLSFDPPHFDSMFAYVKPMNYGPDMVSVYQSLGRVRTLRKGELLIYMDGSGARSEPV

SEQ ID NO: 182 - protein, 116 aa, synthetic construct
ISCLLYDLSTTALEHILLFSLGSCDLPESHLSDLASRGLPAPVVLEFDSEFEMLLAFMTFVKQYGPEFVTGYNIINFDWPFVLTKLTEIYKVPLDGYGRMNGRGVFRVWDIGQSHF

SEQ ID NO: 183 - protein, 55 aa, synthetic construct
GLLPCLHVAATVTTIGREMLLATRAYVHARWAEFDQLLADFPEAAGMRAPGPYSM

SEQ ID NO: 184 - protein, 169 aa, synthetic construct
TSQCPDINHLRSLSILNRWLETELVFVGDEEDVSKLSEGELGFYRFLFAFLSAADDLVTENLGGLSGLFEQKDILHYYVEQECIEVVHSRVYNIIQLVLFHNNDQARRAYVARTINHPAIRVKVDWLEARVRECDSIPEKFILMILIEGVFFAASFAAIAYLRTNNLLR

SEQ ID NO: 185 - protein, 94 aa, synthetic construct
HEFGNLMKVLEYGLPITEEHMQFVDRFVVPESYITNPANLPGWTRLFSSHKEVSAYMAKLHAYLKVTREGEFVVFTLPVLTFVSVKEFDEYRRL

SEQ ID NO: 186 - protein, 99 aa, synthetic construct
ELFGEVFESAPFSTYVDNVIFRGCELLTGSPRGGLMSVALQTDNYTLMGYTYTRVFAFAEELRRRHATAGVAEFLEESPLPYIVLRDQHGFMSVVNTNI

SEQ ID NO: 187 - protein, 184 aa, synthetic construct
SVAAPVEVTALYATDGCVITSSLALLTNCLLGAEPLYIFSYDAYRSDAPNGPTGAPTEQERFEGSRALYRDAGGLNGDSFRVTFCLLGTEVGVTHHPKGRTRPMFVCRFERADDVAVLQDALGRGTPLLPAHVTATLDLEATFALHANIIMALTVAIVHNAPARIGSGSTAPLYEPGESMRSVV

SEQ ID NO: 188 - protein, 75 aa, synthetic construct
RTPADDVSWRYEAPSVIDYARIDGIFLRYHCPGLDTFLWDRHAQRAYLVNPFLFAAGFLEDLSHSVFPADTQETT

SEQ ID NO: 189 - protein, 102 aa, synthetic construct
DGRLLHNTQARAADAADDRPHRPADWTVHHKIYYYVLVPAFSRGRCCTAGVRFDRVYATLQNMVVPEIAPGEECPSDPVTDPAHPLHPANLVANTVKRMFHN

SEQ ID NO: 190 - protein, 55 aa, synthetic construct
SPTQKLAVYYYLIHRERRMSPFPALVRLVGRYIQRHGLYVPAPDEPTLADAMNGL

SEQ ID NO: 191 - protein, 57 aa, synthetic construct
NYTEGIAVVFKENIAPYKFKATMYYKDVTVSQVWFGHRYSQFMGIFEDRAPVPFEEV

SEQ ID NO: 192 - protein, 29 aa, synthetic construct
SVYPYDEFVLATGDFVYMSPFYGYREGSH

SEQ ID NO: 193 - protein, 155 aa, synthetic construct
GLASDPHYDYIRHYASAAKQALGEVELSGGQLSRAILAQYWKYLQTVVPSGLDIPDDPAGDCDPSLHVLLRPTLLPKLLVRAPFKSGAAAAKYAAAVAGLRDAAHRLQQYMFFMRPADPSRPSTDTALRLSELLAYVSVLYHWASWMLWTADKYV

SEQ ID NO: 194 - protein, 70 aa, synthetic construct
GPDAAVFRSSLGSLLYWPGVRALLGRDCRVAARYAGRMTYIATGALLARFNPGAVKCVLPREAAFAGRVL

SEQ ID NO: 195 - protein, 104 aa, synthetic construct
ALFNRLLDDLGFSAGPALCTMLDTWNEDLFSGFPTNADMYRECKFLSTLPSDVIDWGDAHVPERSPIDIRAHGDVAFPTLPATRDELPSYYEAMAQFFRGELRA

SEQ ID NO: 196 - protein, 106 aa, synthetic construct
FLWEDQTLLRATANTITALAVLRRLLANGNVYADRLDNRLQLGMLIPGAVPAEAIARGASGLDSGAIKSGDNNLEALCVNYVLPLYQADPTVELTQLFPGLAALCL

SEQ ID NO: 197 - protein, 758 aa, synthetic construct
MGGAAARLGAVILFVVIVGLHGVRGCTDEIAPPLRCQSFPCLHPFCIPCMKTWIPLRNTCPLCNTPVAYLIVGVTASGSFSTIPIVNDPRTRVEAEAAVRAGTAVDFIWTGNPRTAPRSLSGGSGGGGSGGLPIAGVSSVVALAPYVNKTVTGDCLPVLDMETGHIGAYVVLVDQTGNVADLLRAAAPAWSRRTLLPEHARNCVRPPDYPTPPASEWNSLWMTPVGNMLFDQGTLVGGGSGGGGSGGRAAAWMRQVPDPEDVRVVILYSPLPGEDLAAGRAGGGPPPEWSAERGGLSCLLAALGNRLCGPATAAWAGNWTGAPDVSALGAQGVLLLSTRDLAFAGAVEFLGLLAGACDRRLIVVNAVRAADWPADGPVVSRQHAYLACEVLPAVQCAVRWPAARDLRRTVLASGRVFGPGVFARVEAAHARLYPDAPPLRLCRGANVRYRVRTRFGPDTLVPMSPREYRRAVLPALDGRAAASGGSGGGGSGGETLVAHGPSLYRTFAANPRAASTAKAMRDCVLRQENLIEALASADETLAWCKMCIHHNLPLRPQDPIIGTAAAVLENLATRLRPFLQCYLKARGLCGLDDLCSRRRLSDIKDIASFVLVILARLANRVERGVSEIDYTTVGVGAGETMHFYIPGACMAGLIEILDTHRQECSSRVCELTASHTIAPLYVHGKYFYCNSLFGGSGGGGSGGIVGIVAGLAVLAVVVIGAVVATVMCRRKSSGGKGGSYSQAASSDSAQGSDVSLTA

SEQ ID NO: 198 - protein, 787 aa, synthetic construct
MGGAAARLGAVILFVVIVGLHGVRGREDIETIAFIKRFSLDYGAINFINLAPNNVSELAMYYMANQILRYCDHSTYFINTLTAVIAGSRRPPSVQAAAAWAPQGGAGLEAGARALMDSLDAHPGAWTSMFASCNLLRPVMAARPMVVLGLSISKYYGMAGNDRVFQAGNWASLLGGKNACPLLIFDRTRKFVLGGSGGGGSGGRTFGSAPRLTEDDFGLLNYALAEMRRLCLDLPPVPPNAYTPYHLREYATRLVNGFKPLVRRSARLYRILGVLVHLRIRTREASFEEWMRSKEVDLDFGLTERLREHEAQLMILAQALNPYDCLIHSTPNTLVERGLQSALKYEEFYLKRFGGHYMESVFQMYTRIAGFLAGGSGGGGSGGKMTRGAPKASATPATDPARGRRPAQADSAVLLDAPAPTASGRTKTPAQGLAKKLHFSTAPPSPTAPWTPRVAGFNKRVFCAAVGGGSGGGGSGGLLNNYDVLVLDEVMSTLGQLYSPTMQQLGRVDALMLRLLRTCPRIIAMDATANAQLVDFLCSLRGEKNVHVVIGEYAMPGFSARRCLFLPRLGPEVLQAALRRRGPAGGAPPPDAPPDATFFGELEARLAGGDNVCIFSSTVSFAEVVARFCRQFTDRVLLLHSLTPPGDVTTWGRYRVVIYTTVVTVGLSFDPPHFDSMFAYVKPMNYGPDMVSVYQSLGRVRTLRKGELLIYMDGSGARSEPVGGSGGGGSGGIVGIVAGLAVLAVVVIGAVVATVMCRRKSSGGKGGSYSQAASSDSAQGSDVSLTA

SEQ ID NO: 199 - protein, 863 aa, synthetic construct
MGGAAARLGAVILFVVIVGLHGVRGISCLLYDLSTTALEHILLFSLGSCDLPESHLSDLASRGLPAPVVLEFDSEFEMLLAFMTFVKQYGPEFVTGYNIINFDWPFVLTKLTEIYKVPLDGYGRMNGRGVFRVWDIGQSHFGGSGGGGSGGGLLPCLHVAATVTTIGREMPAGEOFLLATRAYVHARWAEFDQLLADFPEAAGMRAPGPYSMGGSGGGGSGGTSQCPDINHLRSLSILNRWLETELVFVGDEEDVSKLSEGELGFYRFLFAFLSAADDLVTENLGGLSGLFEQKDILHYYVEQECIEVVHSRVYNIIQLVLFHNNDQARRAYVARTINHPAIRVKVDWLEARVRECDSIPEKFILMILIEGVFFAASFAAIAYLRTNNLLRGGSGGGGSGGHEFGNLMKVLEYGLPITEEHMQFVDRFVVPESYITNPANLPGWTRLFSSHKEVSAYMAKLHAYLKVTREGEFVVFTLPVLTFVSVKEFDEYRRLGGSGGGGSGGELFGEVFESAPFSTYVDNVIFRGCELLTGSPRGGLMSVALQTDNYTLMGYTYTRVFAFAEELRRRHATAGVAEFLEESPLPYIVLRDQHGFMSVVNTNIGGSGGGGSGGSVAAPVEVTALYATDGCVITSSLALLTNCLLGAEPLYIFSYDAYRSDAPNGPTGAPTEQERFEGSRALYRDAGGLNGDSFRVTFCLLGTEVGVTHHPKGRTRPMFVCRFERADDVAVLQDALGRGTPLLPAHVTATLDLEATFALHANIIMALTVAIVHNAPARIGSGSTAPLYEPGESMRSVVGGSGGGGSGGIVGIVAGLAVLAVVVIGAVVATVMCRRKSSGGKGGSYSQAASSDSAQGSDVSLTA

SEQ ID NO: 200 - protein, 923 aa, synthetic construct
MGGAAARLGAVILFVVIVGLHGVRGRTPADDVSWRYEAPSVIDYARIDGIFLRYHCPGLDTFLWDRHAQRAYLVNPFLFAAGFLEDLSHSVFPADTQETTGGSGGGGSGGDGRLLHNTQARAADAADDRPHRPADWTVHHKIYYYVLVPAFSRGRCCTAGVRFDRVYATLQNMVVPEIAPGEECPSDPVTDPAHPLHPANLVANTVKRMFHNGGSGGGGSGGSPTQKLAVYYYLIHRERRMSPFPALVRLVGRYIQRHGLYVPAPDEPTLADAMNGLGGSGGGGSGGNYTEGIAVVFKENIAPYKFKATMYYKDVTVSQVWFGHRYSQFMGIFEDRAPVPFEEVGGSGGGGSGGSVYPYDEFVLATGDFVYMSPFYGYREGSHGGSGGGGSGGGLASDPHYDYIRHYASAAKQALGEVELSGGQLSRAILAQYWKYLQTVVPSGLDIPDDPAGDCDPSLHVLLRPTLLPKLLVRAPFKSGAAAAKYAAAVAGLRDAAHRLQQYMFFMRPADPSRPSTDTALRLSELLAYVSVLYHWASWMLWTADKYVGGSGGGGSGGGPDAAVFRSSLGSLLYWPGVRALLGRDCRVAARYAGRMTYIATGALLARFNPGAVKCVLPREAAFAGRVLGGSGGGGSGGFLWEDQTLLRATANTITALAVLRRLLANGNVYADRLDNRLQLGMLIPGAVPAEAIARGASGLDSGAIKSGDNNLEALCVNYVLPLYQADPTVELTQLFPGLAALCLGGSGGGGSGGALFNRLLDDLGFSAGPALCTMLDTWNEDLFSGFPTNADMYRECKFLSTLPSDVIDWGDAHVPERSPIDIRAHGDVAFPTLPATRDELPSYYEAMAQFFRGELRAGGSGGGGSGGIVGIVAGLAVLAVVVIGAVVATVMCRRKSSGGKGGSYSQAASSDSAQGSDVSLTA

SEQ ID NO: 201 - protein, 758 aa, synthetic construct
MGGAAARLGAVILFVVIVGLHGVRGETLVAHGPSLYRTFAANPRAASTAKAMRDCVLRQENLIEALASADETLAWCKMCIHHNLPLRPQDPIIGTAAAVLENLATRLRPFLQCYLKARGLCGLDDLCSRRRLSDIKDIASFVLVILARLANRVERGVSEIDYTTVGVGAGETMHFYIPGACMAGLIEILDTHRQECSSRVCELTASHTIAPLYVHGKYFYCNSLFGGSGGGGSGGRAAAWMRQVPDPEDVRVVILYSPLPGEDLAAGRAGGGPPPEWSAERGGLSCLLAALGNRLCGPATAAWAGNWTGAPDVSALGAQGVLLLSTRDLAFAGAVEFLGLLAGACDRRLIVVNAVRAADWPADGPVVSRQHAYLACEVLPAVQCAVRWPAARDLRRTVLASGRVFGPGVFARVEAAHARLYPDAPPLRLCRGANVRYRVRTRFGPDTLVPMSPREYRRAVLPALDGRAAASGGSGGGGSGGCTDEIAPPLRCQSFPCLHPFCIPCMKTWIPLRNTCPLCNTPVAYLIVGVTASGSFSTIPIVNDPRTRVEAEAAVRAGTAVDFIWTGNPRTAPRSLSGGSGGGGSGGLPIAGVSSVVALAPYVNKTVTGDCLPVLDMETGHIGAYVVLVDQTGNVADLLRAAAPAWSRRTLLPEHARNCVRPPDYPTPPASEWNSLWMTPVGNMLFDQGTLVGGGSGGGGSGGIVGIVAGLAVLAVVVIGAVVATVMCRRKSSGGKGGSYSQAASSDSAQGSDVSLTA

SEQ ID NO: 202 - protein, 787 aa, synthetic construct
MGGAAARLGAVILFVVIVGLHGVRGLLNNYDVLVLDEVMSTLGQLYSPTMQQLGRVDALMLRLLRTCPRIIAMDATANAQLVDFLCSLRGEKNVHVVIGEYAMPGFSARRCLFLPRLGPEVLQAALRRRGPAGGAPPPDAPPDATFFGELEARLAGGDNVCIFSSTVSFAEVVARFCRQFTDRVLLLHSLTPPGDVTTWGRYRVVIYTTVVTVGLSFDPPHFDSMFAYVKPMNYGPDMVSVYQSLGRVRTLRKGELLIYMDGSGARSEPVGGSGGGGSGGKMTRGAPKASATPATDPARGRRPAQADSAVLLDAPAPTASGRTKTPAQGLAKKLHFSTAPPSPTAPWTPRVAGFNKRVFCAAVGGGSGGGGSGGRTFGSAPRLTEDDFGLLNYALAEMRRLCLDLPPVPPNAYTPYHLREYATRLVNGFKPLVRRSARLYRILGVLVHLRIRTREASFEEWMRSKEVDLDFGLTERLREHEAQLMILAQALNPYDCLIHSTPNTLVERGLQSALKYEEFYLKRFGGHYMESVFQMYTRIAGFLAGGSGGGGSGGREDIETIAFIKRFSLDYGAINFINLAPNNVSELAMYYMANQILRYCDHSTYFINTLTAVIAGSRRPPSVQAAAAWAPQGGAGLEAGARALMDSLDAHPGAWTSMFASCNLLRPVMAARPMVVLGLSISKYYGMAGNDRVFQAGNWASLLGGKNACPLLIFDRTRKFVLGGSGGGGSGGIVGIVAGLAVLAVVVIGAVVATVMCRRKSSGGKGGSYSQAASSDSAQGSDVSLTA

SEQ ID NO: 203 - protein, 857 aa, synthetic construct
MGGAAARLGAVILFVVIVGLHGVRGSVAAPVEVTALYATDGCVITSSLALLTNCLLGAEPLYIFSYDAYRSDAPNGPTGAPTEQERFEGSRALYRDAGGLNGDSFRVTFCLLGTEVGVTHHPKGRTRPMFVCRFERADDVAVLQDALGRGTPLLPAHVTATLDLEATFALHANIIMALTVAIVHNAPARIGSGSTAPLYEPGESMRSVVGGSGGGGSGGHEFGNLMKVLEYGLPITEEHMQFVDRFVVPESYITNPANLPGWTRLFSSHKEVSAYMAKLHAYLKVTREGEFVVFTLPVLTFVSVKEFDEYRRLGGSGGGGSGGELFGEVFESAPFSTYVDNVIFRGCELLTGSPRGGLMSVALQTDNYTLMGYTYTRVFAFAEELRRRHATAGVAEFLEESPLPYIVLRDQHGFMSVVNTNIGGSGGGGSGGTSQCPDINHLRSLSILNRWLETELVFVGDEEDVSKLSEGELGFYRFLFAFLSAADDLVTENLGGLSGLFEQKDILHYYVEQECIEVVHSRVYNIIQLVLFHNNDQARRAYVARTINHPAIRVKVDWLEARVRECDSIPEKFILMILIEGVFFAASFAAIAYLRTNNLLRGGSGGGGSGGISCLLYDLSTTALEHILLFSLGSCDLPESHLSDLASRGLPAPVVLEFDSEFEMLLAFMTFVKQYGPEFVTGYNIINFDWPFVLTKLTEIYKVPLDGYGRMNGRGVFRVWDIGQSHFGGSGGGGSGGGLLPCLHVAATVTTIGREMLLATRAYVHARWAEFDQLLADFPEAAGMRAPGPYSMGGSGGGGSGGIVGIVAGLAVLAVVVIGAVVATVMCRRKSSGGKGGSYSQAASSDSAQGSDVSLTA

SEQ ID NO: 204 - protein, 923 aa, synthetic construct
MGGAAARLGAVILFVVIVGLHGVRGALFNRLLDDLGFSAGPALCTMLDTWNEDLFSGFPTNADMYRECKFLSTLPSDVIDWGDAHVPERSPIDIRAHGDVAFPTLPATRDELPSYYEAMAQFFRGELRAGGSGGGGSGGFLWEDQTLLRATANTITALAVLRRLLANGNVYADRLDNRLQLGMLIPGAVPAEAIARGASGLDSGAIKSGDNNLEALCVNYVLPLYQADPTVELTQLFPGLAALCLGGSGGGGSGGGPDAAVFRSSLGSLLYWPGVRALLGRDCRVAARYAGRMTYIATGALLARFNPGAVKCVLPREAAFAGRVLGGSGGGGSGGGLASDPHYDYIRHYASAAKQALGEVELSGGQLSRAILAQYWKYLQTVVPSGLDIPDDPAGDCDPSLHVLLRPTLLPKLLVRAPFKSGAAAAKYAAAVAGLRDAAHRLQQYMFFMRPADPSRPSTDTALRLSELLAYVSVLYHWASWMLWTADKYVGGSGGGGSGGNYTEGIAVVFKENIAPYKFKATMYYKDVTVSQVWFGHRYSQFMGIFEDRAPVPFEEVGGSGGGGSGGSVYPYDEFVLATGDFVYMSPFYGYREGSHGGSGGGGSGGSPTQKLAVYYYLIHRERRMSPFPALVRLVGRYIQRHGLYVPAPDEPTLADAMNGLGGSGGGGSGGDGRLLHNTQARAADAADDRPHRPADWTVHHKIYYYVLVPAFSRGRCCTAGVRFDRVYATLQNMVVPEIAPGEECPSDPVTDPAHPLHPANLVANTVKRMFHNGGSGGGGSGGRTPADDVSWRYEAPSVIDYARIDGIFLRYHCPGLDTFLWDRHAQRAYLVNPFLFAAGFLEDLSHSVFPADTQETTGGSGGGGSGGIVGIVAGLAVLAVVVIGAVVATVMCRRKSSGGKGGSYSQAASSDSAQGSDVSLTA

SEQ ID NO: 205 - protein, 758 aa, synthetic construct
MGRLTSGVGTAALLVVAVGLRVVCACTDEIAPPLRCQSFPCLHPFCIPCMKTWIPLRNTCPLCNTPVAYLIVGVTASGSFSTIPIVNDPRTRVEAEAAVRAGTAVDFIWTGNPRTAPRSLSGGSGGGGSGGLPIAGVSSVVALAPYVNKTVTGDCLPVLDMETGHIGAYVVLVDQTGNVADLLRAAAPAWSRRTLLPEHARNCVRPPDYPTPPASEWNSLWMTPVGNMLFDQGTLVGGGSGGGGSGGRAAAWMRQVPDPEDVRVVILYSPLPGEDLAAGRAGGGPPPEWSAERGGLSCLLAALGNRLCGPATAAWAGNWTGAPDVSALGAQGVLLLSTRDLAFAGAVEFLGLLAGACDRRLIVVNAVRAADWPADGPVVSRQHAYLACEVLPAVQCAVRWPAARDLRRTVLASGRVFGPGVFARVEAAHARLYPDAPPLRLCRGANVRYRVRTRFGPDTLVPMSPREYRRAVLPALDGRAAASGGSGGGGSGGETLVAHGPSLYRTFAANPRAASTAKAMRDCVLRQENLIEALASADETLAWCKMCIHHNLPLRPQDPIIGTAAAVLENLATRLRPFLQCYLKARGLCGLDDLCSRRRLSDIKDIASFVLVILARLANRVERGVSEIDYTTVGVGAGETMHFYIPGACMAGLIEILDTHRQECSSRVCELTASHTIAPLYVHGKYFYCNSLFGGSGGGGSGGIVGIVAGLAVLAVVVIGAVVATVMCRRKSSGGKGGSYSQAASSDSAQGSDVSLTA

SEQ ID NO: 206 - protein, 758 aa, synthetic construct
MGRLTSGVGTAALLVVAVGLRVVCAETLVAHGPSLYRTFAANPRAASTAKAMRDCVLRQENLIEALASADETLAWCKMCIHHNLPLRPQDPIIGTAAAVLENLATRLRPFLQCYLKARGLCGLDDLCSRRRLSDIKDIASFVLVILARLANRVERGVSEIDYTTVGVGAGETMHFYIPGACMAGLIEILDTHRQECSSRVCELTASHTIAPLYVHGKYFYCNSLFGGSGGGGSGGRAAAWMRQVPDPEDVRVVILYSPLPGEDLAAGRAGGGPPPEWSAERGGLSCLLAALGNRLCGPATAAWAGNWTGAPDVSALGAQGVLLLSTRDLAFAGAVEFLGLLAGACDRRLIVVNAVRAADWPADGPVVSRQHAYLACEVLPAVQCAVRWPAARDLRRTVLASGRVFGPGVFARVEAAHARLYPDAPPLRLCRGANVRYRVRTRFGPDTLVPMSPREYRRAVLPALDGRAAASGGSGGGGSGGCTDEIAPPLRCQSFPCLHPFCIPCMKTWIPLRNTCPLCNTPVAYLIVGVTASGSFSTIPIVNDPRTRVEAEAAVRAGTAVDFIWTGNPRTAPRSLSGGSGGGGSGGLPIAGVSSVVALAPYVNKTVTGDCLPVLDMETGHIGAYVVLVDQTGNVADLLRAAAPAWSRRTLLPEHARNCVRPPDYPTPPASEWNSLWMTPVGNMLFDQGTLVGGGSGGGGSGGIVGIVAGLAVLAVVVIGAVVATVMCRRKSSGGKGGSYSQAASSDSAQGSDVSLTA

SEQ ID NO: 207 - RNA, 12 nt, synthetic construct
  source 1..12: mol_type = other RNA
aaaaaaattttt

SEQ ID NO: 208 - RNA, 53 nt, synthetic construct
  source 1..53: mol_type = other RNA
agaataaactagtattcttctggtccccacagactcagagagaacccgccacc

SEQ ID NO: 209 - RNA, 47 nt, synthetic construct
  source 1..47: mol_type = other RNA
aactagtattcttctggtccccacagactcagagagaacccgccacc

SEQ ID NO: 210 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 211 - skipped

SEQ ID NO: 212 - RNA, 110 nt, synthetic construct; identical to SEQ ID NO: 210
  source 1..110: mol_type = other RNA

SEQ ID NO: 213 - RNA, 110 nt, synthetic construct; identical to SEQ ID NO: 210
  source 1..110: mol_type = other RNA

SEQ ID NO: 214 - skipped

SEQ ID NO: 215 - RNA, 278 nt, synthetic construct
  source 1..278: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 216 - RNA, 278 nt, synthetic construct; identical to SEQ ID NO: 215
  source 1..278: mol_type = other RNA

SEQ ID NO: 217 - protein, 6 aa, synthetic construct
GSGSGS

SEQ ID NO: 218 - RNA, 75 nt, Human alphaherpesvirus 1
  source 1..75: mol_type = other RNA; strain = Strain F
atggggggggctgccgccaggttgggggccgtgattttgtttgtcgtcatagtcggcctccatggggtccgcggc

SEQ ID NO: 219 - RNA, 75 nt, Human alphaherpesvirus 1
  source 1..75: mol_type = other RNA; strain = Strain F
atggggcgtttgacctccggcgtcgggacggcggccctgctagttgtcgcggtgggactccgcgtcgtctgcgcc

SEQ ID NO: 220 - protein, 28 aa, Human alphaherpesvirus 1
  source 1..28: note = HSV-1 strain KOS
MGGAAARLGAVILFVVIVGLHGVRGKYA

SEQ ID NO: 221 - protein, 331 aa, Human alphaherpesvirus 2
MGRLTSGVGTAALLVVAVGLRVVCAKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHH

SEQ ID NO: 222 - protein, 453 aa, Human alphaherpesvirus 2
  source 1..453: note = HSV-2 strain 333
SPGRTITVGPRGNASNAAPSASPRNASAPRTTPTPPQPRKATKSKASTAKPAPPPKTGPPKTSSEPVRCNRHDPLARYGSRVQIRCRFPNSTRTESRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRLPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHHGSHQPPPRDPTERQVIRAVEGAGIGVAVLVAVVLAGTAVVYLTHASSVRYRRLR

SEQ ID NO: 223 - protein, 4 aa, synthetic construct
FRAC

SEQ ID NO: 224 - protein, 4 aa, synthetic construct
KRCF

SEQ ID NO: 225 - protein, 4 aa, synthetic construct
KKRY

SEQ ID NO: 226 - protein, 4 aa, synthetic construct
ARMA

SEQ ID NO: 227 - protein, 4 aa, synthetic construct
RRSG

SEQ ID NO: 228 - protein, 4 aa, synthetic construct
MRAC

SEQ ID NO: 229 - protein, 4 aa, synthetic construct
KMCG

SEQ ID NO: 230 - protein, 4 aa, synthetic construct
ARCA

SEQ ID NO: 231 - protein, 4 aa, synthetic construct
KKQG

SEQ ID NO: 232 - protein, 4 aa, synthetic construct
YRSY

SEQ ID NO: 233 - protein, 4 aa, synthetic construct
SFMN

SEQ ID NO: 234 - protein, 4 aa, synthetic construct
FKAA

SEQ ID NO: 235 - protein, 4 aa, synthetic construct
KRNG

SEQ ID NO: 236 - protein, 4 aa, synthetic construct
YNSF

SEQ ID NO: 237 - protein, 4 aa, synthetic construct
KKNG

SEQ ID NO: 238 - protein, 4 aa, synthetic construct
RRRG

SEQ ID NO: 239 - protein, 4 aa, synthetic construct
KRYS

SEQ ID NO: 240 - protein, 4 aa, synthetic construct
ARYA

SEQ ID NO: 241 - protein, 4 aa, synthetic construct
GGSL